(19) United States
(12) Patent Application Publication
(10) Pub. No.: US 2023/0019491 A1
(43) Pub. Date: Jan. 19, 2023

(54) PYRIMIDINE TBK/IKKe INHIBITOR COMPOUNDS AND USES THEREOF

(71) Applicants: Srinivasa R. KARRA, Pembroke, MA (US); YuFang XIAO, Lexington, MA (US); Brian A. SHERER, Nashua, NH (US); Merck Patent GmbH, Darmstadt (DE)

(72) Inventors: Srinivasa R. Karra, Pembroke, MA (US); Yufang XIAO, Lexington, MA (US); Brian Sherer, Nashua, NH (US)

(21) Appl. No.: 16/756,769

(22) PCT Filed: Oct. 17, 2018
(86) PCT No.: PCT/US18/56190

§ 371 Date: Apr. 16, 2020

Related U.S. Application Data
(60) Provisional application No. 62/573,251, filed on Oct. 17, 2017.

(51) Int. Cl. C07D 405/14; C07D 417/14; C07D 498/08; C07D 493/08; C07D 491/048; C07D 453/02; C07D 401/14; C07D 413/14

(52) U.S. Cl. CPC C07D 405/14; C07D 417/14; C07D 498/08; C07D 413/14; C07D 491/048; C07D 453/02; C07D 401/14; C07D 493/08

(57) ABSTRACT

The present invention relates to compounds of Formula I and pharmaceutically acceptable compositions thereof, useful as TBK/IKKε inhibitors.

### RELATED APPLICATIONS

This application claims the benefit of U.S. Provisional Application 62/573,251, filed on Oct. 17, 2017. The entire content of the aforementioned application is incorporated herein by reference.

### TECHNICAL FIELD OF THE INVENTION

The present invention provides for compounds of Formula (I) as dual inhibitors of TBK and IKKε that can be used to treat immunological disorders, TBK and/or IKKε inhibitors and their use in the treatment of cancer, and other diseases related to TBK and/or IKKε overexpression, including rheumatoid arthritis, systemic lupus erythematosus or lupus nephritis.

### BACKGROUND OF THE INVENTION

Protein kinases regulate nearly every cellular process, including metabolism, cell proliferation, cell differentiation, and cell survival, so they are attractive targets for therapeutic intervention for various disease states. For example, cell-cycle control and angiogenesis, in which protein kinases play a pivotal role are cellular processes associated with numerous disease conditions such as but not limited to cancer, inflammatory diseases, abnormal angiogenesis and diseases related thereto, atherosclerosis, macular degeneration, diabetes, obesity, and pain.

One of the principal mechanisms by which cellular regulation is effected is through the transduction of extracellular signals across the membrane that in turn modulate biochemical pathways within the cell. Protein phosphorylation represents one course by which intracellular signals are propagated from molecule to molecule resulting finally in a cellular response. These signal transduction cascades are highly regulated and often overlap, as is evident from the existence of many protein kinases as well as phosphatases. Phosphorylation of proteins occurs predominantly at serine, threonine or tyrosine residues, and protein kinases have therefore been classified by their specificity of phosphorylation site, i.e. serine/threonine kinases and tyrosine kinases. Since phosphorylation is such a ubiquitous process within cells and since cellular phenotypes are largely influenced by the activity of these pathways, it is currently believed that a number of disease states and/or diseases are attributable to either aberrant activation or functional mutations in the molecular components of kinase cascades. Consequently, considerable attention has been devoted to the characterisation of these proteins and compounds that are able to modulate their activity (for a review see: Weinstein-Oppenheimer et al. Pharma. &. Therap., 2000, 88, 229-279).

IKKε and TBK1 are serine/threonine kinases which are highly homologous to one another and other IkB kinases. The two kinases play an integral role in the innate immune system. Double-stranded RNA viruses are recognised by the Toll-like receptors 3 and 4 and the RNA helicases RIG-I and MDA-5 and result in activation of the TRIF-TBK1/IKKE-IRF3 signalling cascade, which results in a type I interferon response.

In 2007, Boehm et al. described IKKE as a novel breast cancer oncogene (J. S. Boehm et al., Cell 129, 1065-1079, 2007). 354 kinases were investigated with respect to their ability to recapitulate the Ras-transforming phenotype together with an activated form of the MAPK kinase Mek. IKKE was identified here as a cooperative oncogene. In addition, the authors were able to show that IKKE is amplified and overexpressed in numerous breast cancer cell lines and tumour samples. The reduction in gene expression by means of RNA interference in breast cancer cells induces apoptosis and impairs the proliferation thereof. Eddy et al. obtained similar findings in 2005, which underlines the importance of IKKε in breast cancer diseases (S. F. Eddy et al., Cancer Res. 2005; 65 (24), 11375-11383).

A protumorigenic effect of TBK1 was reported for the first time in 2006. In a screening of a gene library comprising 251,000 cDNA, Korherr et al. identified precisely three genes, TRIF, TBK1 and IRF3, which are typically involved in the innate immune defence as proangiogenic factors (C. Korherr et al., PNAS, 103, 4240-4245, 2006). In 2006, Chien et al. (Y. Chien et al., Cell 127, 157-170, 2006) published that TBK1−/− cells can only be transformed to a limited extent using oncogenic Ras, which suggests an involvement of TBK1 in the Ras-mediated transformation. Furthermore, they were able to show that an RNAi-mediated knockdown of TBK1 triggers apoptosis in MCF-7 and Panc-1 cells. Barbie et al. recently published that TBK1 is of essential importance in numerous cancer cell lines with mutated K-Ras, which suggests that TBK1 intervention could be of therapeutic importance in corresponding tumours (D. A. Barbie et al., Nature Letters 1-5, 2009).

Diseases caused by protein kinases are characterised by anomalous activity or hyperactivity of such protein kinases. Anomalous activity relates to either (1) expression in cells which do not usually express these protein kinases; (2) increased kinase expression, which results in undesired cell proliferation, such as cancer, (3) increased kinase activity, which results in undesired cell proliferation, such as cancer, and/or in hyperactivity of the corresponding protein kinases. Hyperactivity relates either to amplification of the gene which encodes for a certain protein kinase, or the generation of an activity level which can be correlated with a cell proliferation disease (i.e. the severity of one or more symptoms of the cell proliferation disease increases with increasing kinase level). The bioavailability of a protein kinase may also be influenced by the presence or absence of a set of binding proteins of this kinase.

IKKε and TBK1 are highly homologous Ser/Thr kinases critically involved in the innate immune response through induction of type 1 interferons and other cytokines. These kinases are stimulated in response to viral/bacterial infection. Immune response to viral and bacterial infection involves the binding of antigens such as bacterial lipopolysaccharide (LPS), viral doublestranded RNS (dsRNA) to Toll like receptors, then subsequent activation of TBK1 pathway. Activated TBK1 and IKKε phosphorylate IRF3 and IRF7, which triggers the dimerization and nuclear translocation of those interferon regulatory transcription factors, ultimately inducing a signaling cascades leading to IFN production.

### SUMMARY OF THE INVENTION

In one aspect, the invention provides a compound of Formula (I):

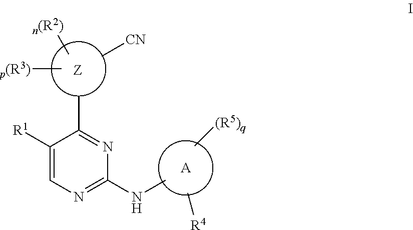

or a pharmaceutically acceptable derivative, solvate, salt, hydrate or stereoisomer thereof.

In another aspect, the invention provides compounds of Formula (I) which are suitable as a dual inhibitor of TBK and IKKε. The compounds of the invention have high solubility and high bioavailability.

In another aspect, the invention provides methods for the treatment and/or prevention of immunological disorders related to TBK and IKKε comprising administering a compound of Formula (I). In another aspect, the invention provides compounds which are able to modulate, especially inhibit the activity or function of TBK and IKKε in disease states in mammals.

In certain embodiments, the present invention provides compounds of Formula (I) which are selective for TBK and/or IKKε. In certain embodiments, the present invention provides compounds of Formula (I) which are selective for TBK and IKKε.

### DETAILED DESCRIPTION OF CERTAIN EMBODIMENTS

### 1. General Description of Compounds of the Invention

In certain aspects, the present invention provides for dual inhibitors of TBK and IKKε. In some embodiments, such compounds include those of the formulae described herein, or a pharmaceutically acceptable salt thereof, wherein each variable is as defined and described herein.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

The term “lower alkyl” refers to a CM straight or branched alkyl group. Exemplary lower alkyl groups are methyl, ethyl, propyl, isopropyl, butyl, isobutyl, and tert-butyl.

The term “lower haloalkyl” refers to a CM straight or branched alkyl group that is substituted with one or more halogen atoms.

The term “heteroatom” means one or more of oxygen, sulfur, nitrogen, or phosphorus (including, any oxidized form of nitrogen, sulfur, or phosphorus; the quaternized form of any basic nitrogen or, a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or CM) saturated or unsaturated, straight or branched, hydrocarbon chain”, refers to bivalent alkylene, alkenylene, and alkynylene chains that are straight or branched as defined herein.

According to the invention, bivalent groups include substitution in both directions, and when inserted between any two groups, (e.g., the group “—OC(O)—” or “CO₂” inserted between X and Y), includes both

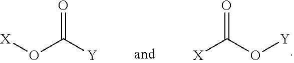

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 TE electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or ⁺NR (as in/V-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds of the invention contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

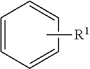

refers to at least

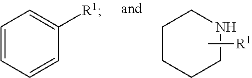

refers to at least

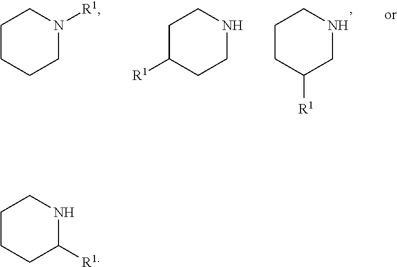

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which are optionally substituted with R^{∘}; —(CH₂)₀₋₄O(CH₂)₀₋₁Ph which is optionally substituted with R^{∘}; —CH═CHPh, which is optionally substituted with R^{∘}; —(CH₂)₀₋₄O(CH₂)₀₋₁-pyridyl which is optionally substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} is optionally substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which is optionally substituted as defined below.

Suitable monovalent substituents on R^{∘} (or the ring formed by taking two independent occurrences of R^{∘} together with their intervening atoms), are independently deuterium, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}3, —OSiR^{●}₃, —C(O)SR^{●}. —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, −O(C(R*₂))₂₋₃O—, or —S(C(R*₂))₂₋₃S—, wherein each independent occurrence of R* is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{●}₂)₂₋₃O—, wherein each independent occurrence of R^{●} is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:
—F, —Cl, —Br, —I, deuterium,
—OH, protected hydroxy, alkoxy, oxo, thiooxo,
—NO₂, —CN, CF₃, N₃,
—NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino,
—O— alkyl, —O— alkenyl, —O— alkynyl, —O— cycloalkyl, —O-aryl, —O-heteroaryl, —O-heterocyclic,
—C(O)— alkyl, —C(O)— alkenyl, —C(O)— alkynyl, —C(O)— carbocyclyl, —C(O)-aryl, —C(O)— heteroaryl, —C(O)-heterocyclyl,
—CONH₂, —CONH— alkyl, —CONH— alkenyl, —CONH— alkynyl, —CONH-carbocyclyl, —CONH-aryl, —CONH-heteroaryl, —CONH-heterocyclyl,
—OCO₂— alkyl, —OCO₂— alkenyl, —OCO₂— alkynyl, —OCO₂— carbocyclyl, —OCO₂-aryl, —OCO₂-heteroaryl, —OCO₂-heterocyclyl, —OCONH₂, —OCONH— alkyl, —OCONH— alkenyl, —OCONH— alkynyl, —OCONH— carbocyclyl, —OCONH— aryl, —OCONH— heteroaryl, —OCONH— heterocyclyl,
—NHC(O)— alkyl, —NHC(O)— alkenyl, —NHC(O)— alkynyl, —NHC(O)— carbocyclyl, —NHC(O)-aryl, —NHC(O)-heteroaryl, —NHC(O)-heterocyclyl, —NHCO₂— alkyl, —NHCO₂— alkenyl, —NHCO₂— alkynyl, —NHCO₂— carbocyclyl, —NHCO₂— aryl, —NHCO₂— heteroaryl, —NHCO₂— heterocyclyl, —NHC(O)NH₂, —NHC(O)NH— alkyl, —NHC(O)NH— alkenyl, —NHC(O)NH— alkenyl, —NHC(O)NH— carbocyclyl, —NHC(O)NH-aryl, —NHC(O)NH-heteroaryl, —NHC(O)NH— heterocyclyl, NHC(S)NH₂, —NHC(S)NH— alkyl, —NHC(S)NH— alkenyl, —NHC(S)NH— alkynyl, —NHC(S)NH— carbocyclyl, —NHC(S)NH-aryl, —NHC(S)NH-heteroaryl, —NHC(S)NH-heterocyclyl, —NHC(NH)NH₂, —NHC(NH)NH— alkyl, —NHC(NH)NH— -alkenyl, —NHC(NH)NH— alkenyl, —NHC(NH)NH— carbocyclyl, —NHC(NH)NH-aryl, —NHC(NH)NH-heteroaryl, —NHC(NH)NH— heterocyclyl, —NHC(NH)— alkyl, —NHC(NH)— alkenyl, —NHC(NH)— alkenyl, —NHC(NH)— carbocyclyl, —NHC(NH)-aryl, —NHC(NH)-heteroaryl, —NHC(NH)-heterocyclyl,
—C(NH)NH— alkyl, —C(NH)NH— alkenyl, —C(NH)NH— alkynyl, —C(NH)NH— carbocyclyl, —C(NH)NH-aryl, —C(NH)NH-heteroaryl, —C(NH)NH-heterocyclyl,
—S(O)— alkyl, —S(O)— alkenyl, —S(O)— alkynyl, —S(O)— carbocyclyl, —S(O)-aryl, —S(O)-heteroaryl, —S(O)-heterocyclyl —SO₂NH₂, —SO₂NH— alkyl, —SO₂NH— alkenyl, —SO₂NH— alkynyl, —SO₂NH— carbocyclyl, —SO₂NH— aryl, —SO₂NH— heteroaryl, —SO₂NH— heterocyclyl,
—NHSO₂— alkyl, —NHSO₂— alkenyl, —NHSO₂— alkynyl, —NHSO₂— carbocyclyl, —NHSO₂-aryl, —NHSO₂-heteroaryl, —NHSO₂-heterocyclyl,
—CH₂NH₂, —CH₂SO₂CH₃, -mono-, di-, or tri-alkyl silyl, -alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, -methoxymethoxy, -methoxyethoxy, —SH, —S— alkyl, —S— alkenyl, —S— alkynyl, —S— carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

Deuterium (²H) can also be incorporated into a compound of the formula I for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a corn-pound of the formula I that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of the formula I with improved stability through resistance to such oxidative metabolism. Significant improvements in the pharmacokinetic profiles of compounds of the formula I are thereby obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

As used herein, the term “modulator” is defined as a compound that binds to and/or inhibits the target with measurable affinity. In certain embodiments, a modulator has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in TBK and/or IKKε activity between a sample comprising a compound of the present invention, or composition thereof, and TBK and/or IKKε, and an equivalent sample comprising TBK and/or IKKε, in the absence of said compound, or composition thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### 3. Description of Exemplary Compounds

According to one aspect, the present invention provides a compound of formula I,

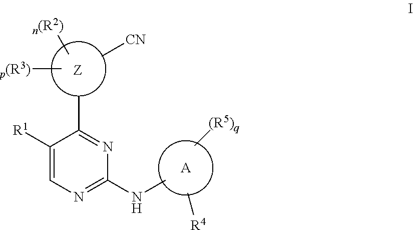

or pharmaceutically acceptable derivatives, solvates, salts, hydrates, or stereoisomers thereof, wherein:
R¹ is hydrogen, optionally substituted C₁₋₆ aliphatic, —OR, or halogen;
ring Z is phenyl or a 5-6-membered heteroaryl having 1, 2, or 3 nitrogens;
each R² is independently —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
each R³ is independently —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
ring A is phenyl or a 5-6-membered heteroaryl having 1, 2, or 3 nitrogens; R⁴ is —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, a 5-6 membered monocyclic heteroaryl ring having 1-4 heteroatoms independently selected from nitrogen, oxygen, or sulfur, or a 6-12 membered spiro, fused, or bridged bicyclic carbocyclic or heterocyclic ring having 1-4 heteroatoms independently selected from nitrogen, oxygen, or sulfur, each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted;
n is 1 or 2;
p is 0, 1, or 2; and
q is 0, 1, or 2.

In certain embodiments, R¹ is H.

In certain embodiments, R¹ is optionally substituted C₁₋₆ aliphatic, —OR, or halogen.

In certain embodiments, R¹ is C₁₋₆ aliphatic.

In certain embodiments, R¹ is methyl, ethyl, propyl, i-propyl, butyl, s-butyl, t-butyl, straight chain or branched pentyl, or straight chain or branched hexyl, each of which is optionally substituted. In certain embodiments, R¹ is methyl. In certain embodiments, R¹ is i-propyl.

In certain embodiments, R¹ is —OR. In certain embodiments, R¹ is —OMe.

In certain embodiments, R¹ is halogen.

In certain embodiments, R¹ is F or Cl.

In certain embodiments, R¹ is H or F.

In certain embodiments, ring Z is phenyl, pyridine, or pyrimidine.

In certain embodiments, ring Z is phenyl.

In certain embodiments, ring Z is pyridine.

In certain embodiments, ring Z is pyrimidine.

In certain embodiments, ring Z is

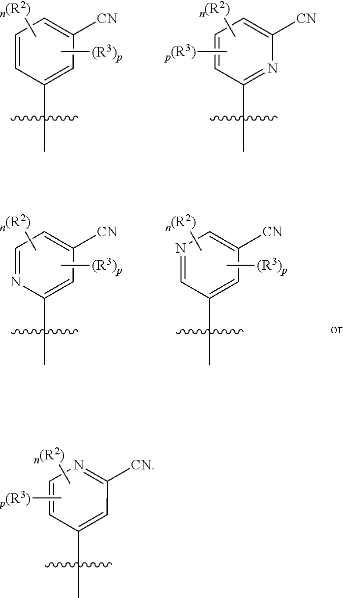

In certain embodiments, ring Z is

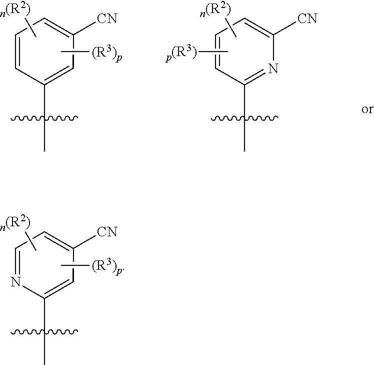

In certain embodiments, each R² is independently —R, halogen, —OR, or —N(R)₂.

In certain embodiments, each R² is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted; or R² is halogen, —OR, or —N(R)₂.

In certain embodiments, each R² is independently methyl, ethyl, propyl, i-propyl, butyl, s-butyl, t-butyl, straight chain or branched pentyl, or straight chain or branched hexyl; each of which is optionally substituted.

In certain embodiments, each R² is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R² is independently F, Cl, Br, or I.

In certain embodiments, each R² is independently —OR, or —N(R)₂.

In certain embodiments, each R² is independently

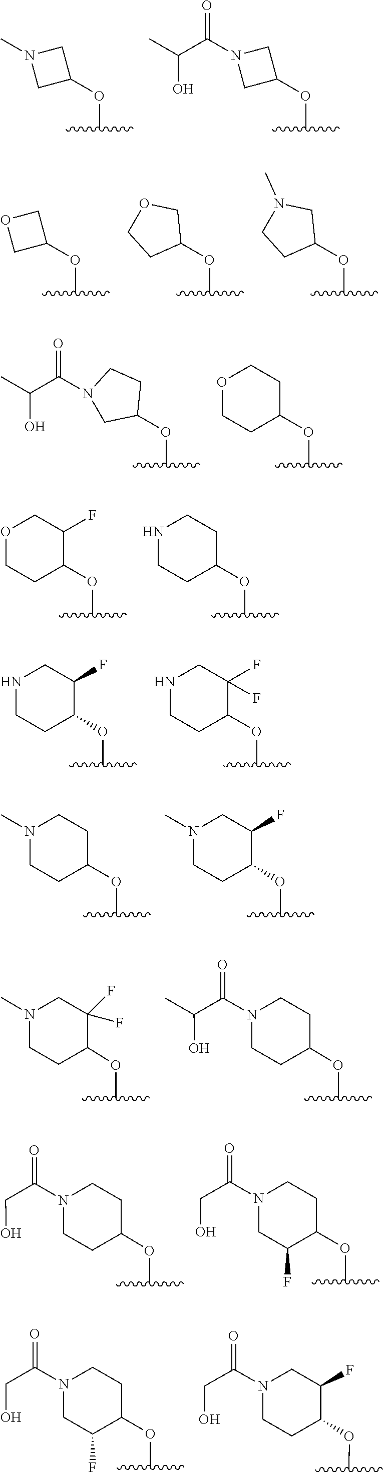

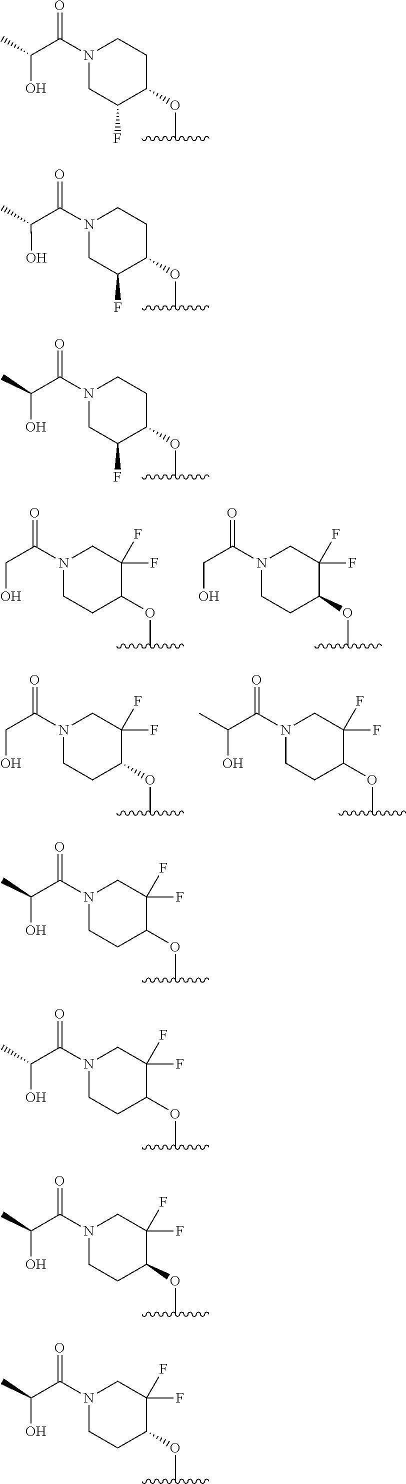

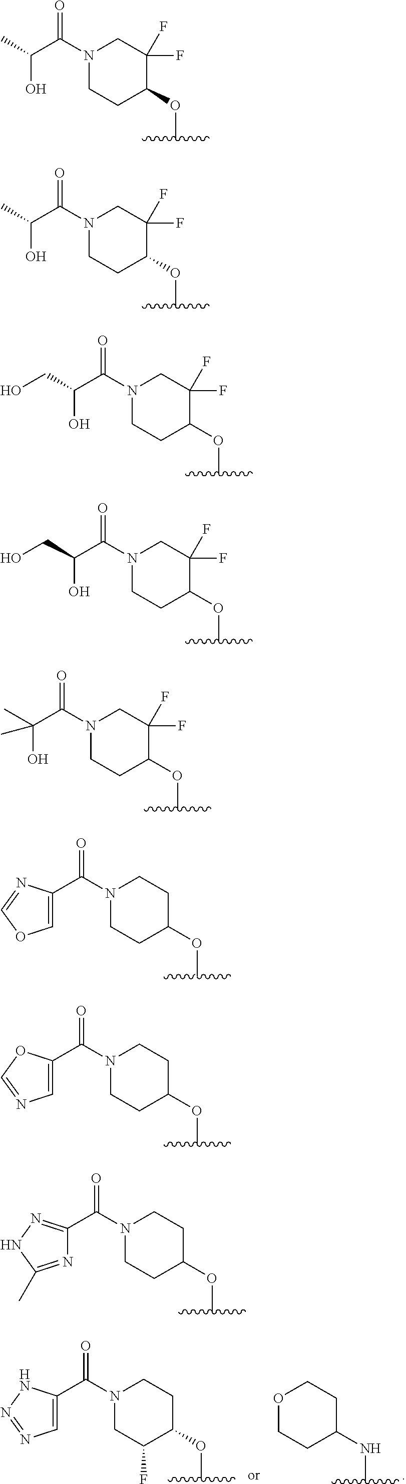

In certain embodiments, each R³ is independently —R, halogen, —OR, or —N(R)₂.

In certain embodiments, each R³ is independently H.

In certain embodiments, ring A is phenyl or pyridyl.

In certain embodiments, ring A is pyridyl.

In certain embodiments, ring A is

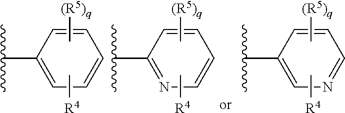

In certain embodiments, ring A is

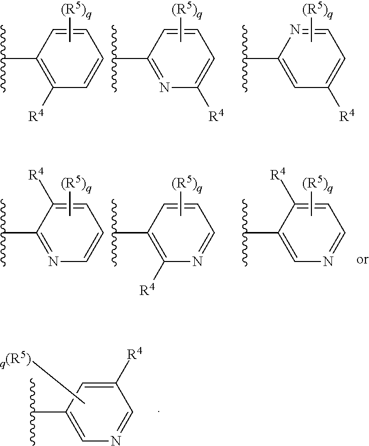

In certain embodiments, R⁴ is —R, halogen, —OR, —NRC(O)R, —NRC(O)N(R)₂, —NRSO₂R, or —N(R)₂. In certain embodiments, R⁴ is —R or —OR.

In certain embodiments, R⁴ is H.

In certain embodiments, R⁴ is —OR, wherein R is H, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted.

In certain embodiments, R⁴ is —H, —OH, —OCH₃, or —OCF₃.

In certain embodiments, each R⁵ is independently —R, OR, —C(O)R, —CO₂R, —C(O)N(R)₂, —NRC(O)R, —NRC(O)N(R)₂, —NRSO₂R, or —N(R)₂.

In certain embodiments, each R⁵ is independently —R, —C(O)R, —CO₂R, —C(O)N(R)₂, —NRC(O)R, or —N(R)₂.

In certain embodiments, each R⁵ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted; or R² is halogen, —OR, or —N(R)₂.

In certain embodiments, each R⁵ is independently methyl, ethyl, propyl, i-propyl, butyl, s-butyl, t-butyl, straight chain or branched pentyl, or straight chain or branched hexyl; each of which is optionally substituted.

In certain embodiments, each R⁵ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁵ is independently —R, —C(O)R, —CO₂R, —C(O)N(R)₂, —NRC(O)R, or —N(R)₂.

In certain embodiments, each R⁵ is independently

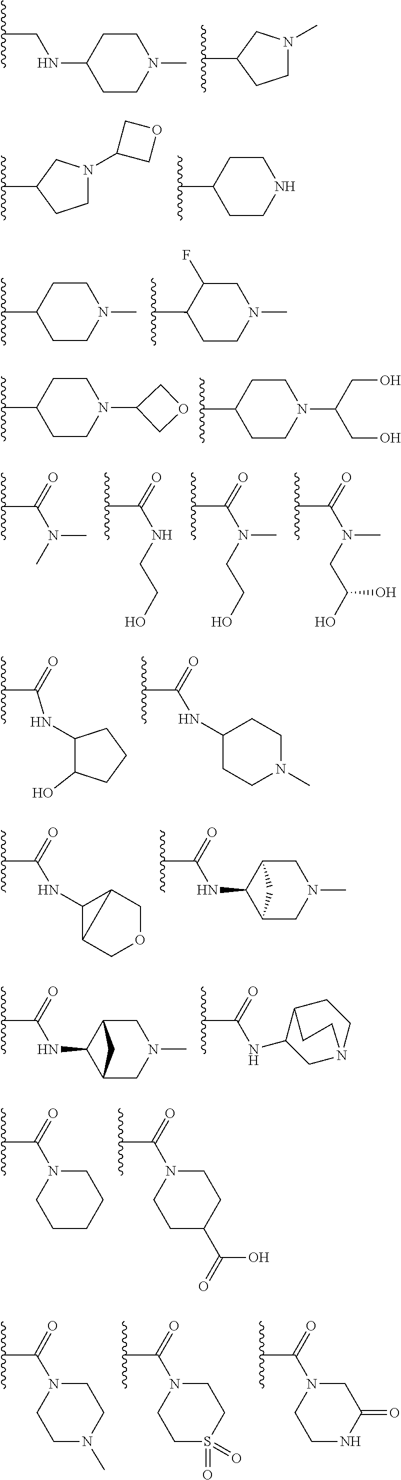

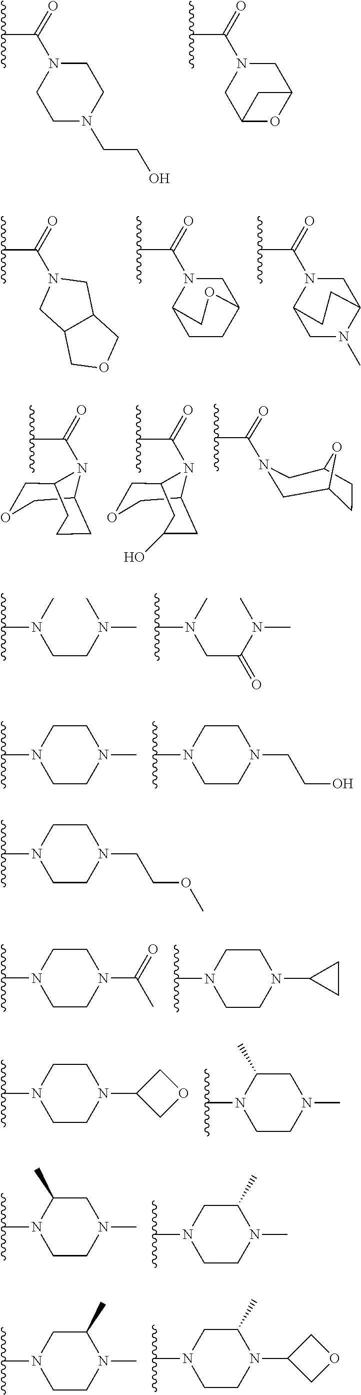

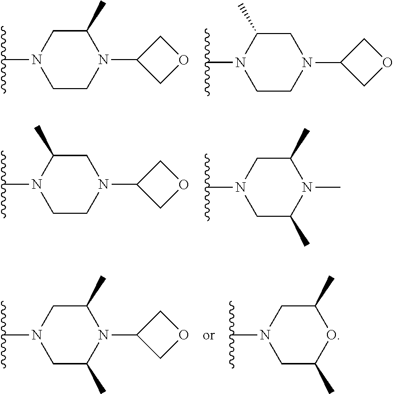

In certain embodiments, each of Ring A, Ring Z, R, R¹, R², R³, R⁴, R⁵, n, p, and q, is as defined above and described in embodiments, classes and subclasses above and herein, singly or in combination.

In certain embodiments, the present invention provides a compound of formula II,

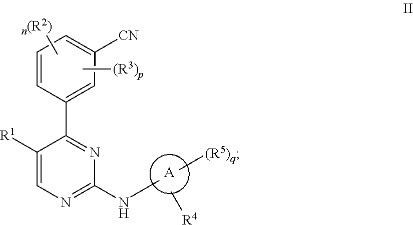

or a pharmaceutically acceptable salt thereof, wherein each of ring A, R¹, R², R³, R⁴, R⁵, n, p, and q, is as defined above and described in embodiments, classes and subclasses above and herein, singly or in combination.

In certain embodiments, the present invention provides a compound of formula III,

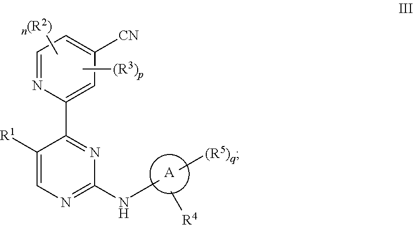

or a pharmaceutically acceptable salt thereof, wherein each of ring A, R¹, R², R³, R⁴, R⁵, n, p, and q, is as defined above and described in embodiments, classes and subclasses above and herein, singly or in combination.

In certain embodiments, the present invention provides a compound of formula IV,

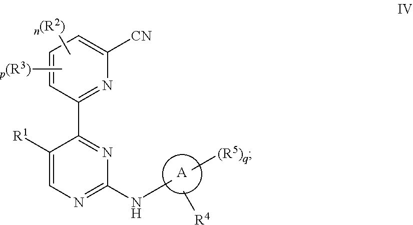

or a pharmaceutically acceptable salt thereof, wherein each of ring A, R¹, R², R³, R⁴, R⁵, n, p, and q, is as defined above and described in embodiments, classes and subclasses above and herein, singly or in combination.

In certain embodiments, the present invention provides a compound of formula V,

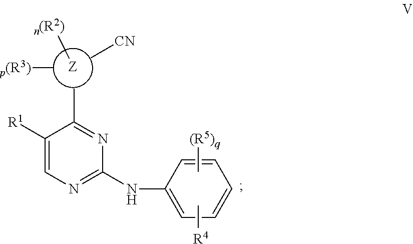

or a pharmaceutically acceptable salt thereof, wherein each of ring Z, R¹, R², R³, R⁴, R⁵, n, p, and q, is as defined above and described in embodiments, classes and subclasses above and herein, singly or in combination.

In certain embodiments, the present invention provides a compound of formula VI,

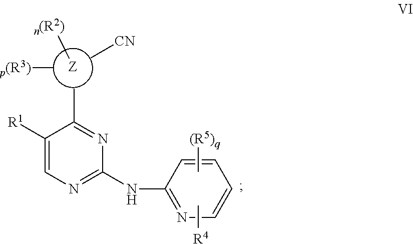

or a pharmaceutically acceptable salt thereof, wherein each of ring Z, R¹, R², R³, R⁴, R⁵, n, p, and q, is as defined above and described in embodiments, classes and subclasses above and herein, singly or in combination.

In certain embodiments, the present invention provides a compound of formula VII,

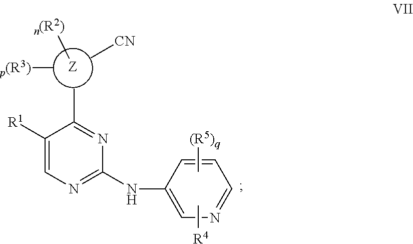

or a pharmaceutically acceptable salt thereof, wherein each of ring Z, R¹, R², R³, R⁴, R⁵, n, p, and q, is as defined above and described in embodiments, classes and subclasses above and herein, singly or in combination.

In certain embodiments, the invention provides a compound selected from Table 1:

TABLE 1

| | |
|---|---|
| 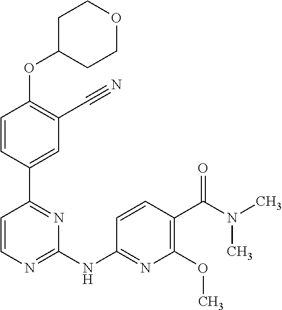 [SMILES: COc1nc(Nc2nccc(-c3ccc(OC4CCOCC4)c(C#N)c3)n2)ccc1C(=O)N(C)C] | 1 |
| 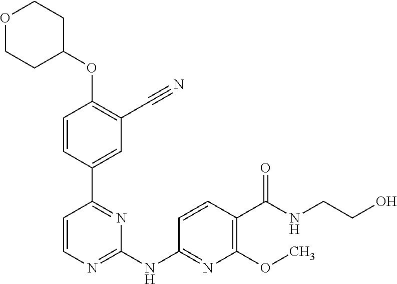 [SMILES: COc1nc(Nc2nccc(-c3ccc(OC4CCOCC4)c(C#N)c3)n2)ccc1C(=O)NCCO] | 2 |
| 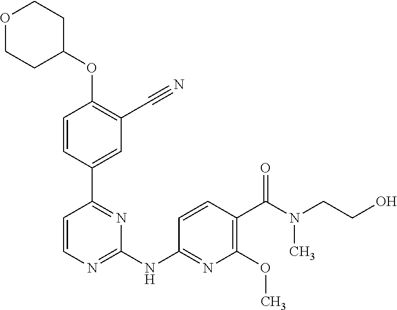 [SMILES: COc1nc(Nc2nccc(-c3ccc(OC4CCOCC4)c(C#N)c3)n2)ccc1C(=O)N(C)CCO] | 3 |
| 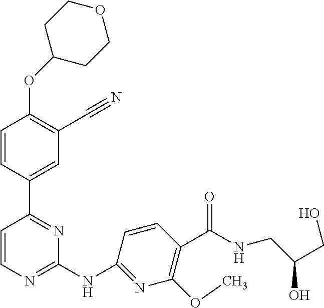 [SMILES: COc1nc(Nc2nccc(-c3ccc(OC4CCOCC4)c(C#N)c3)n2)ccc1C(=O)NC[C@H](O)CO] | 4 |
| 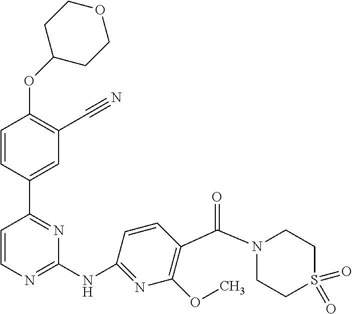 [SMILES: COc1nc(Nc2nccc(-c3ccc(OC4CCOCC4)c(C#N)c3)n2)ccc1C(=O)N1CCS(=O)(=O)CC1] | 5 |
| 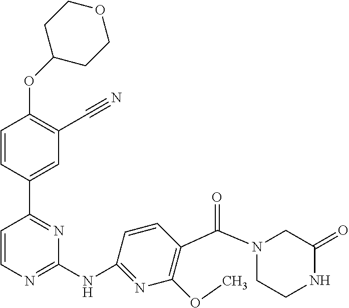 [SMILES: COc1nc(Nc2nccc(-c3ccc(OC4CCOCC4)c(C#N)c3)n2)ccc1C(=O)N1CCNC(=O)C1] | 6 |
| 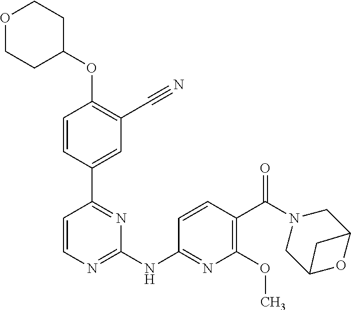 [SMILES: COc1nc(Nc2nccc(-c3ccc(OC4CCOCC4)c(C#N)c3)n2)ccc1C(=O)N1CC2CC(C1)O2] | 7 |
| 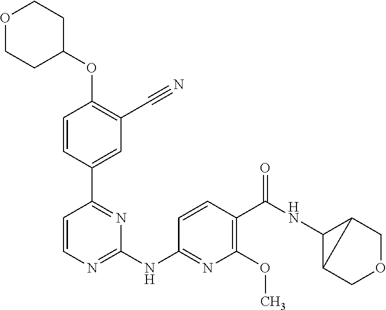 [SMILES: COc1nc(Nc2nccc(-c3ccc(OC4CCOCC4)c(C#N)c3)n2)ccc1C(=O)NC1C2COCC21] | 8 |
| 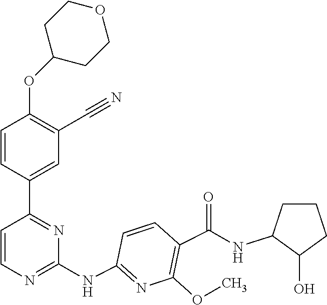 [SMILES: COc1nc(Nc2nccc(-c3ccc(OC4CCOCC4)c(C#N)c3)n2)ccc1C(=O)NC1CCCC1O] | 9 |
| 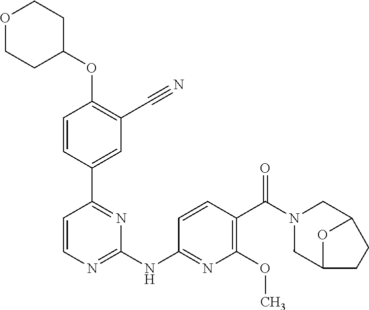 [SMILES: COc1nc(Nc2nccc(-c3ccc(OC4CCOCC4)c(C#N)c3)n2)ccc1C(=O)N1CC2CCC(C1)O2] | 10 |
| 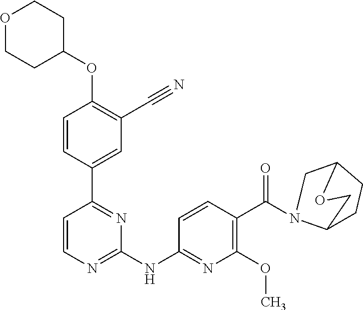 [SMILES: COc1nc(Nc2nccc(-c3ccc(OC4CCOCC4)c(C#N)c3)n2)ccc1C(=O)N1CC2CCC1CO2] | 11 |
| 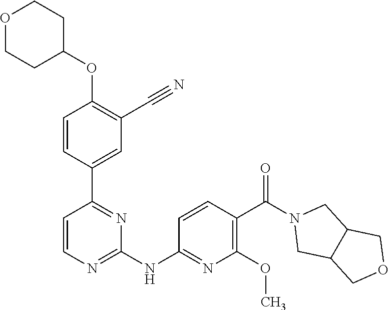 [SMILES: COc1nc(Nc2nccc(-c3ccc(OC4CCOCC4)c(C#N)c3)n2)ccc1C(=O)N1CC2COCC2C1] | 12 |
| 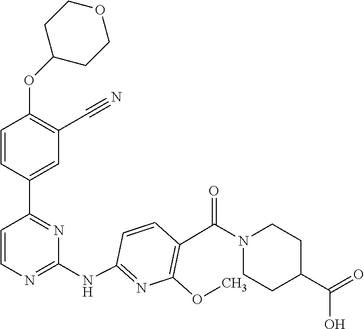 [SMILES: COc1nc(Nc2nccc(-c3ccc(OC4CCOCC4)c(C#N)c3)n2)ccc1C(=O)N1CCC(C(=O)O)CC1] | 13 |
| 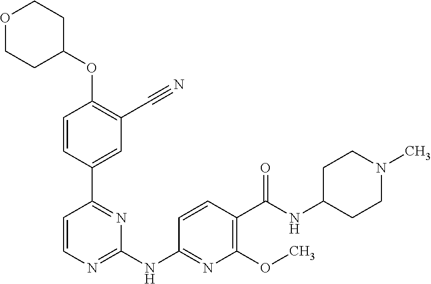 [SMILES: COc1nc(Nc2nccc(-c3ccc(OC4CCOCC4)c(C#N)c3)n2)ccc1C(=O)NC1CCN(C)CC1] | 14 |
| 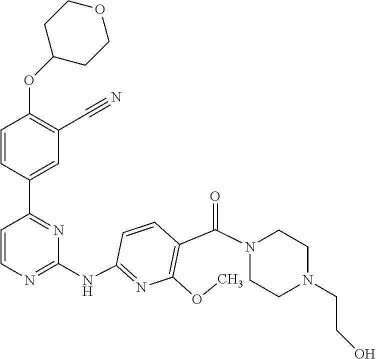 [SMILES: COc1nc(Nc2nccc(-c3ccc(OC4CCOCC4)c(C#N)c3)n2)ccc1C(=O)N1CCN(CCO)CC1] | 15 |
| 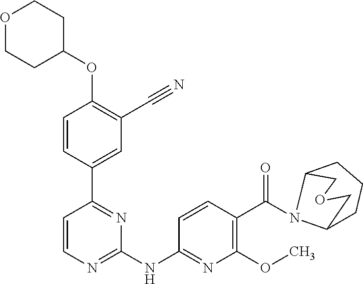 [SMILES: COc1nc(Nc2nccc(-c3ccc(OC4CCOCC4)c(C#N)c3)n2)ccc1C(=O)N1C2CCCC1COC2] | 16 |
| 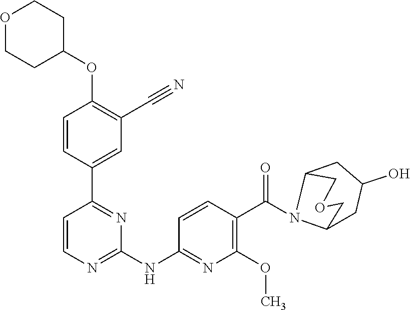 [SMILES: COc1nc(Nc2nccc(-c3ccc(OC4CCOCC4)c(C#N)c3)n2)ccc1C(=O)N1C2COCC1CC(O)C2] | 17 |
| 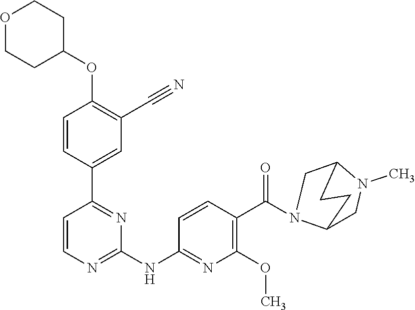 [SMILES: COc1nc(Nc2nccc(-c3ccc(OC4CCOCC4)c(C#N)c3)n2)ccc1C(=O)N1CC2CCC1CN2C] | 18 |
| 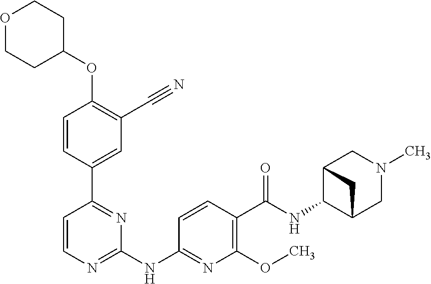 [SMILES: COc1nc(Nc2nccc(-c3ccc(OC4CCOCC4)c(C#N)c3)n2)ccc1C(=O)N[C@H]1[C@@H]2C[C@H]1CN(C)C2] | 19 |
| 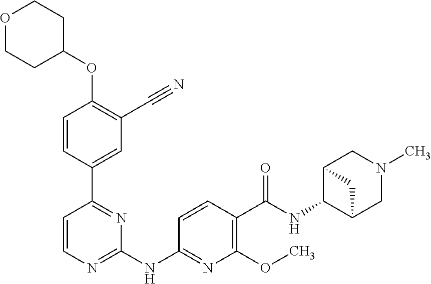 [SMILES: COc1nc(Nc2nccc(-c3ccc(OC4CCOCC4)c(C#N)c3)n2)ccc1C(=O)N[C@@H]1[C@@H]2C[C@H]1CN(C)C2] | 20 |
| 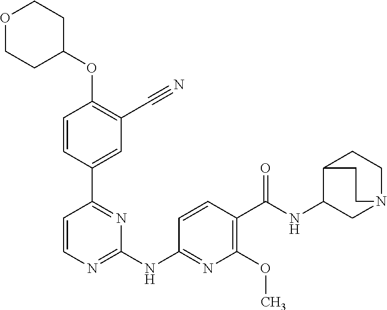 [SMILES: COc1nc(Nc2nccc(-c3ccc(OC4CCOCC4)c(C#N)c3)n2)ccc1C(=O)NC1CN2CCC1CC2] | 21 |
| 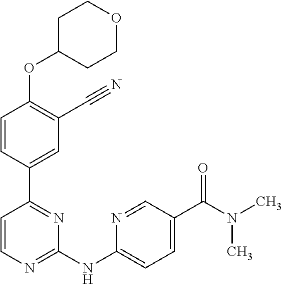 [SMILES: CN(C)C(=O)c1ccc(Nc2nccc(-c3ccc(OC4CCOCC4)c(C#N)c3)n2)nc1] | 22 |
| 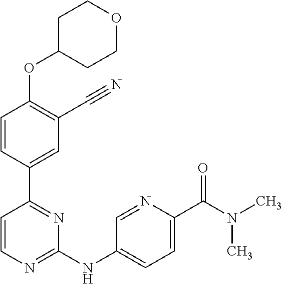 [SMILES: CN(C)C(=O)c1ccc(Nc2nccc(-c3ccc(OC4CCOCC4)c(C#N)c3)n2)cn1] | 23 |
| 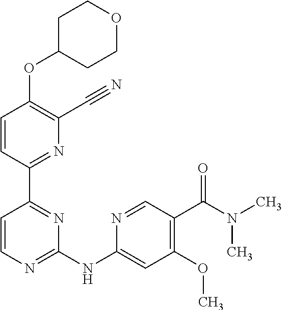 [SMILES: COc1cc(Nc2nccc(-c3ccc(OC4CCOCC4)c(C#N)n3)n2)ncc1C(=O)N(C)C] | 24 |
| 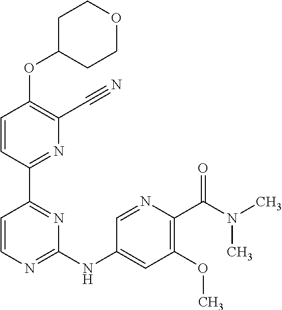 [SMILES: COc1cc(Nc2nccc(-c3ccc(OC4CCOCC4)c(C#N)n3)n2)cnc1C(=O)N(C)C] | 25 |
| 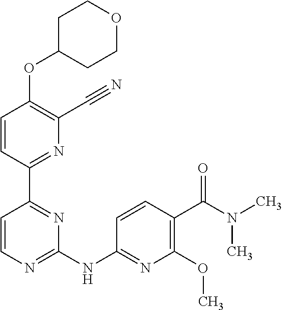 [SMILES: COc1nc(Nc2nccc(-c3ccc(OC4CCOCC4)c(C#N)n3)n2)ccc1C(=O)N(C)C] | 26 |
| 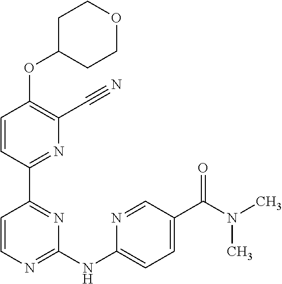 [SMILES: CN(C)C(=O)c1ccc(Nc2nccc(-c3ccc(OC4CCOCC4)c(C#N)n3)n2)nc1] | 27 |
| 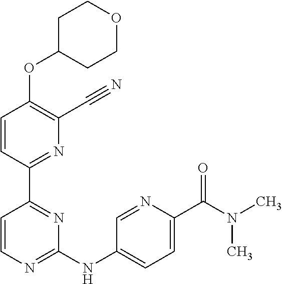 [SMILES: CN(C)C(=O)c1ccc(Nc2nccc(-c3ccc(OC4CCOCC4)c(C#N)n3)n2)cn1] | 28 |
| 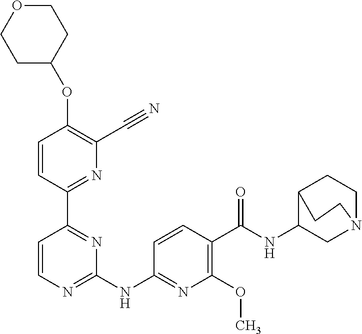 [SMILES: COc1nc(Nc2nccc(-c3ccc(OC4CCOCC4)c(C#N)n3)n2)ccc1C(=O)NC1CN2CCC1CC2] | 29 |
| 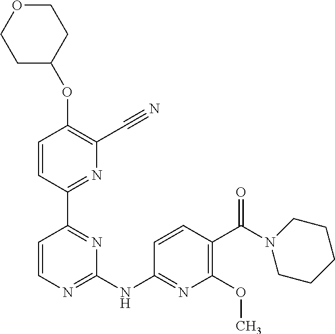 [SMILES: COc1nc(Nc2nccc(-c3ccc(OC4CCOCC4)c(C#N)n3)n2)ccc1C(=O)N1CCCCC1] | 30 |
| 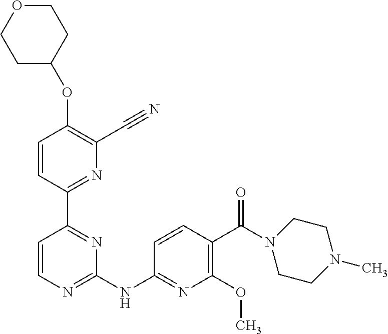 [SMILES: COc1nc(Nc2nccc(-c3ccc(OC4CCOCC4)c(C#N)n3)n2)ccc1C(=O)N1CCN(C)CC1] | 31 |
| 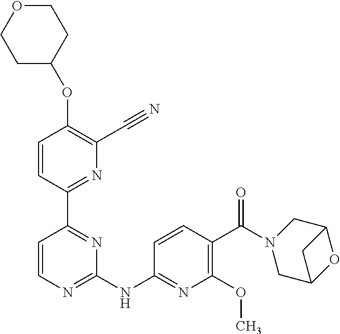 [SMILES: COc1nc(Nc2nccc(-c3ccc(OC4CCOCC4)c(C#N)n3)n2)ccc1C(=O)N1CC2CC(C1)O2] | 32 |
| 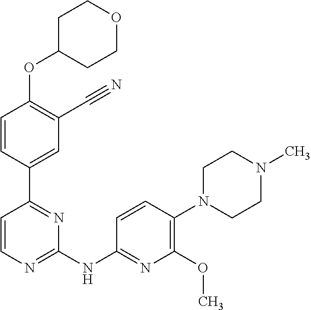 [SMILES: COc1nc(Nc2nccc(-c3ccc(OC4CCOCC4)c(C#N)c3)n2)ccc1N1CCN(C)CC1] | 33 |
| 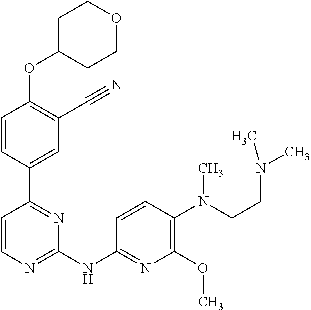 [SMILES: COc1nc(Nc2nccc(-c3ccc(OC4CCOCC4)c(C#N)c3)n2)ccc1N(C)CCN(C)C] | 34 |
| 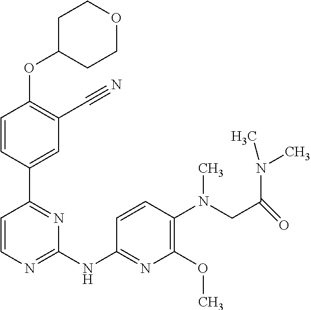 [SMILES: COc1nc(Nc2nccc(-c3ccc(OC4CCOCC4)c(C#N)c3)n2)ccc1N(C)CC(=O)N(C)C] | 35 |
| 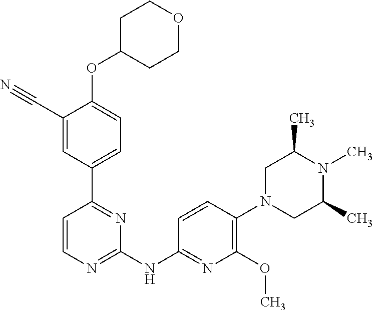 [SMILES: COc1nc(Nc2nccc(-c3ccc(OC4CCOCC4)c(C#N)c3)n2)ccc1N1C[C@@H](C)N(C)[C@@H](C)C1] | 36 |
| 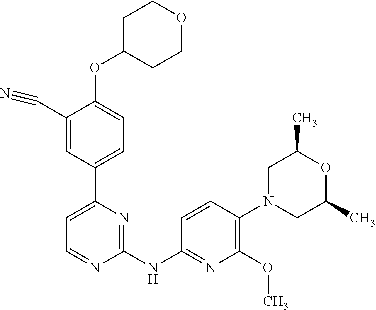 [SMILES: COc1nc(Nc2nccc(-c3ccc(OC4CCOCC4)c(C#N)c3)n2)ccc1N1C[C@@H](C)O[C@@H](C)C1] | 37 |
| 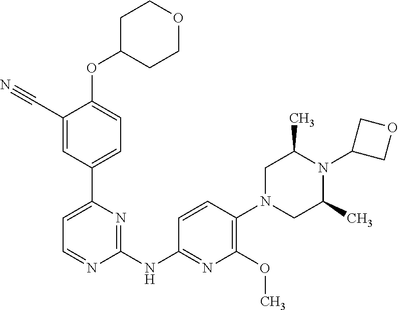 [SMILES: COc1nc(Nc2nccc(-c3ccc(OC4CCOCC4)c(C#N)c3)n2)ccc1N1C[C@@H](C)N(C2COC2)[C@@H](C)C1] | 38 |
| 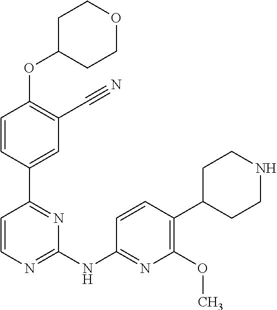 [SMILES: COc1nc(Nc2nccc(-c3ccc(OC4CCOCC4)c(C#N)c3)n2)ccc1C1CCNCC1] | 39 |
| 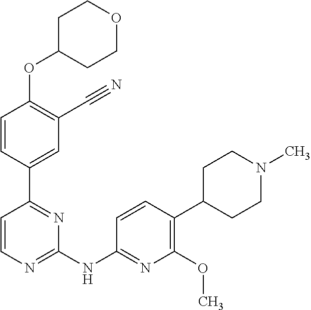 [SMILES: COc1nc(Nc2nccc(-c3ccc(OC4CCOCC4)c(C#N)c3)n2)ccc1C1CCN(C)CC1] | 40 |
| 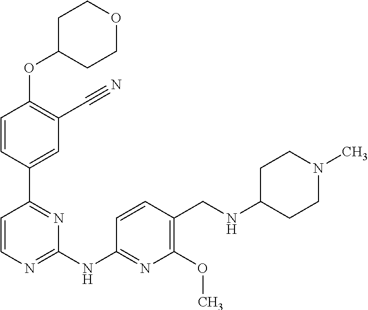 [SMILES: COc1nc(Nc2nccc(-c3ccc(OC4CCOCC4)c(C#N)c3)n2)ccc1CNC1CCN(C)CC1] | 41 |
| 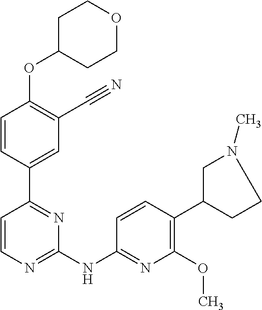 [SMILES: COc1nc(Nc2nccc(-c3ccc(OC4CCOCC4)c(C#N)c3)n2)ccc1C1CCN(C)C1] | 42 |
| 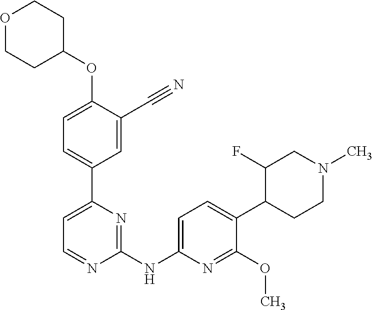 [SMILES: COc1nc(Nc2nccc(-c3ccc(OC4CCOCC4)c(C#N)c3)n2)ccc1C1CCN(C)CC1F] | 43 |
| 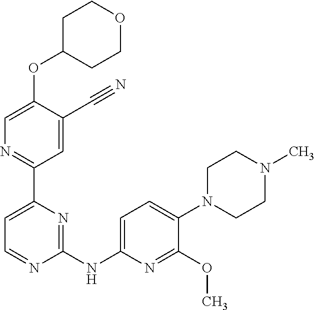 [SMILES: COc1nc(Nc2nccc(-c3cc(C#N)c(OC4CCOCC4)cn3)n2)ccc1N1CCN(C)CC1] | 44 |
| 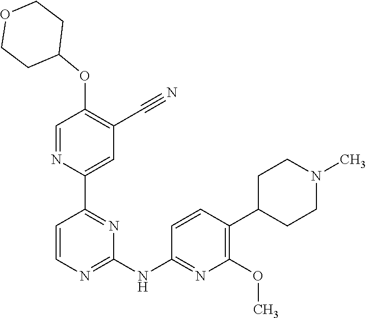 [SMILES: COc1nc(Nc2nccc(-c3cc(C#N)c(OC4CCOCC4)cn3)n2)ccc1C1CCN(C)CC1] | 45 |
| 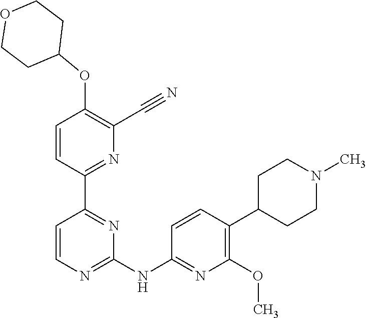 [SMILES: COc1nc(Nc2nccc(-c3ccc(OC4CCOCC4)c(C#N)n3)n2)ccc1C1CCN(C)CC1] | 46 |
| 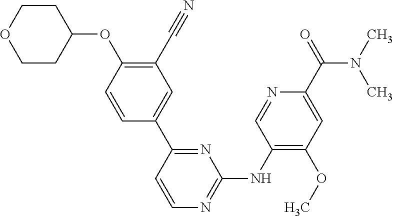 [SMILES: COc1cc(C(=O)N(C)C)ncc1Nc1nccc(-c2ccc(OC3CCOCC3)c(C#N)c2)n1] | 47 |
| 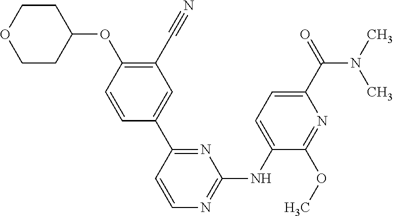 [SMILES: COc1nc(C(=O)N(C)C)ccc1Nc1nccc(-c2ccc(OC3CCOCC3)c(C#N)c2)n1] | 48 |
| 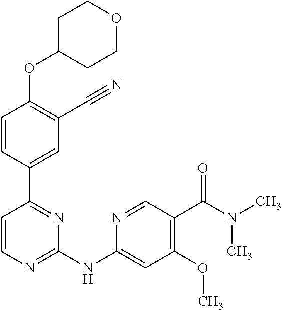 [SMILES: COc1cc(Nc2nccc(-c3ccc(OC4CCOCC4)c(C#N)c3)n2)ncc1C(=O)N(C)C] | 49 |
| 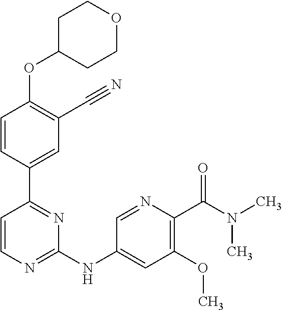 [SMILES: COc1cc(Nc2nccc(-c3ccc(OC4CCOCC4)c(C#N)c3)n2)cnc1C(=O)N(C)C] | 50 |
| 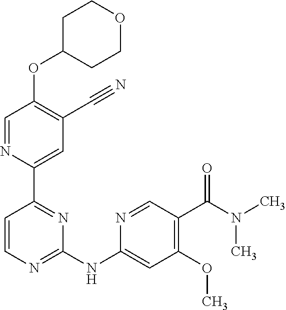 [SMILES: COc1cc(Nc2nccc(-c3cc(C#N)c(OC4CCOCC4)cn3)n2)ncc1C(=O)N(C)C] | 51 |
| 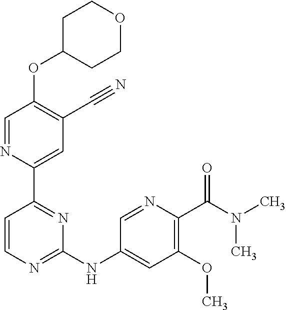 [SMILES: COc1cc(Nc2nccc(-c3cc(C#N)c(OC4CCOCC4)cn3)n2)cnc1C(=O)N(C)C] | 52 |
| 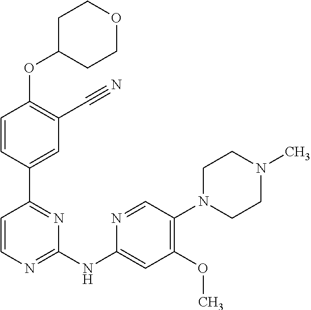 [SMILES: COc1cc(Nc2nccc(-c3ccc(OC4CCOCC4)c(C#N)c3)n2)ncc1N1CCN(C)CC1] | 53 |
| 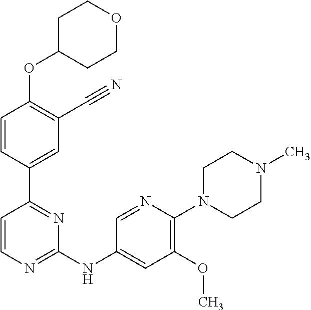 [SMILES: COc1cc(Nc2nccc(-c3ccc(OC4CCOCC4)c(C#N)c3)n2)cnc1N1CCN(C)CC1] | 54 |
| 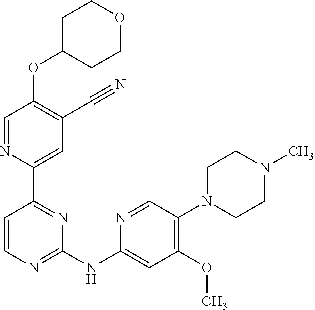 [SMILES: COc1cc(Nc2nccc(-c3cc(C#N)c(OC4CCOCC4)cn3)n2)ncc1N1CCN(C)CC1] | 55 |
| 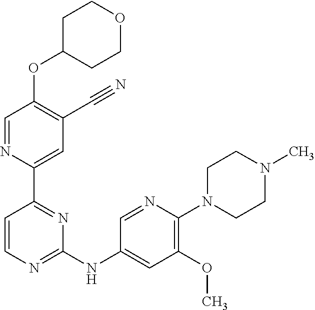 [SMILES: COc1cc(Nc2nccc(-c3cc(C#N)c(OC4CCOCC4)cn3)n2)cnc1N1CCN(C)CC1] | 56 |
| 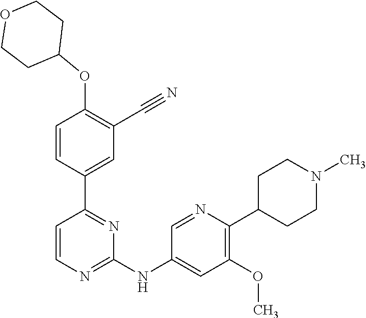 [SMILES: COc1cc(Nc2nccc(-c3ccc(OC4CCOCC4)c(C#N)c3)n2)cnc1C1CCN(C)CC1] | 57 |
| 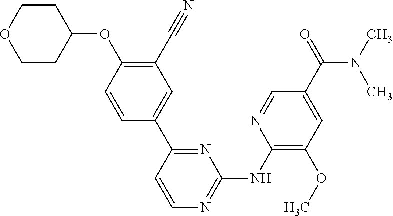 [SMILES: COc1cc(C(=O)N(C)C)cnc1Nc1nccc(-c2ccc(OC3CCOCC3)c(C#N)c2)n1] | 58 |
| 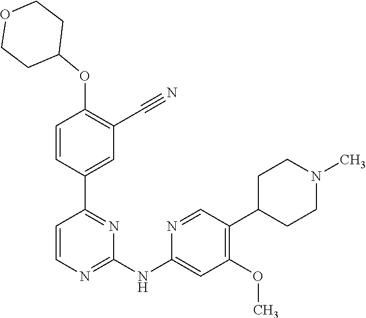 [SMILES: COc1cc(Nc2nccc(-c3ccc(OC4CCOCC4)c(C#N)c3)n2)ncc1C1CCN(C)CC1] | 59 |
| 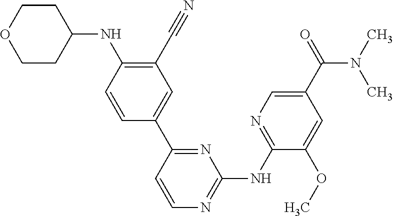 [SMILES: COc1cc(C(=O)N(C)C)cnc1Nc1nccc(-c2ccc(NC3CCOCC3)c(C#N)c2)n1] | 60 |
| 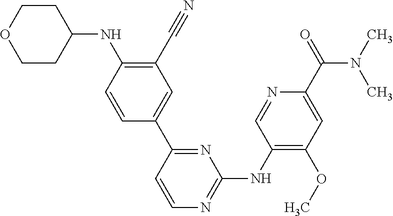 [SMILES: COc1cc(C(=O)N(C)C)ncc1Nc1nccc(-c2ccc(NC3CCOCC3)c(C#N)c2)n1] | 61 |
| 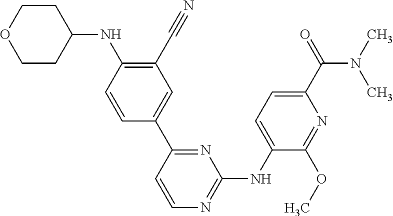 [SMILES: COc1nc(C(=O)N(C)C)ccc1Nc1nccc(-c2ccc(NC3CCOCC3)c(C#N)c2)n1] | 62 |
| 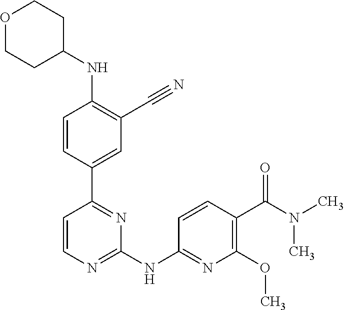 [SMILES: COc1nc(Nc2nccc(-c3ccc(NC4CCOCC4)c(C#N)c3)n2)ccc1C(=O)N(C)C] | 63 |
| 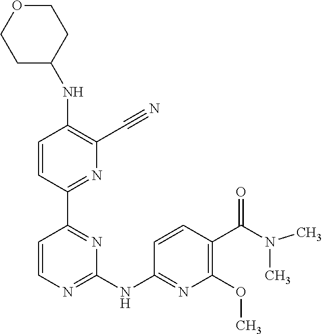 [SMILES: COc1nc(Nc2nccc(-c3ccc(NC4CCOCC4)c(C#N)n3)n2)ccc1C(=O)N(C)C] | 64 |
| 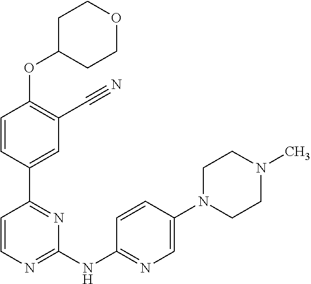 [SMILES: CN1CCN(c2ccc(Nc3nccc(-c4ccc(OC5CCOCC5)c(C#N)c4)n3)nc2)CC1] | 65 |
| 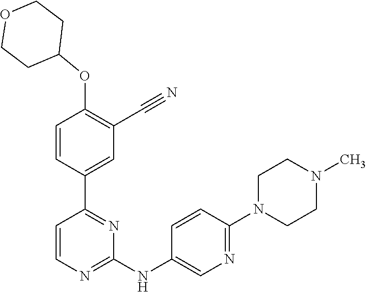 [SMILES: CN1CCN(c2ccc(Nc3nccc(-c4ccc(OC5CCOCC5)c(C#N)c4)n3)cn2)CC1] | 66 |
| 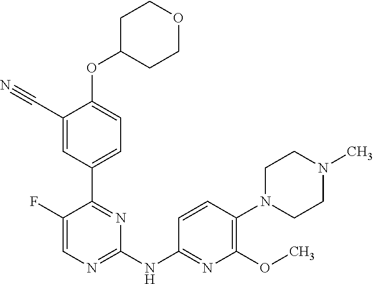 [SMILES: COc1nc(Nc2ncc(F)c(-c3ccc(OC4CCOCC4)c(C#N)c3)n2)ccc1N1CCN(C)CC1] | 67 |
| 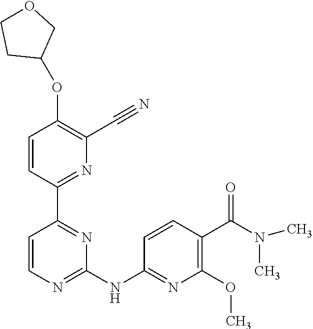 [SMILES: COc1nc(Nc2nccc(-c3ccc(OC4CCOC4)c(C#N)n3)n2)ccc1C(=O)N(C)C] | 68 |
| 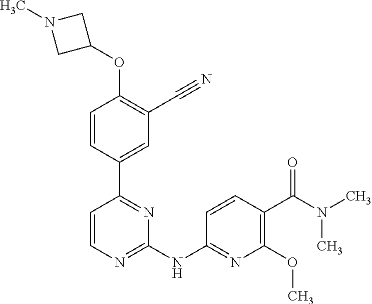 | 69 |
| 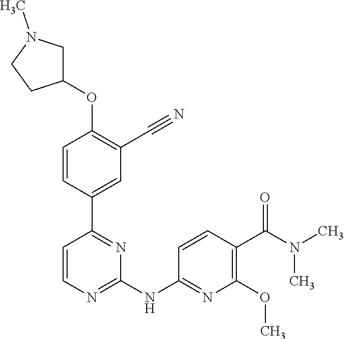 [SMILES: COc1nc(Nc2nccc(-c3ccc(OC4CCN(C)C4)c(C#N)c3)n2)ccc1C(=O)N(C)C] | 70 |
| 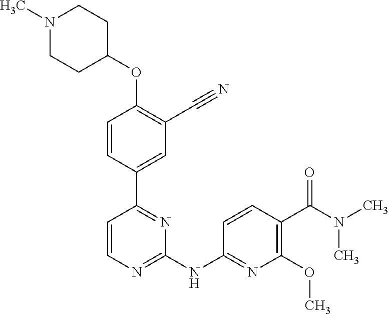 | 71 |
| 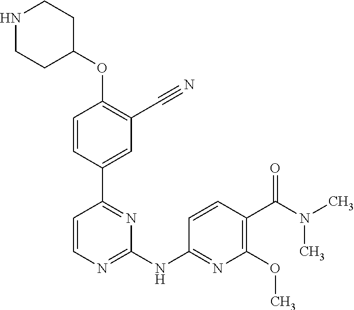 [SMILES: COc1nc(Nc2nccc(-c3ccc(OC4CCNCC4)c(C#N)c3)n2)ccc1C(=O)N(C)C] | 72 |
| 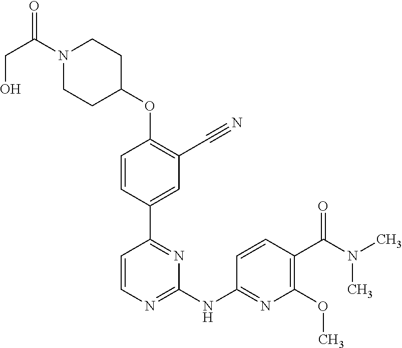 [SMILES: COc1nc(Nc2nccc(-c3ccc(OC4CCN(C(=O)CO)CC4)c(C#N)c3)n2)ccc1C(=O)N(C)C] | 73 |
| 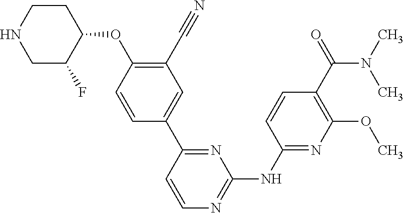 [SMILES: COc1nc(Nc2nccc(-c3ccc(O[C@H]4CCNC[C@H]4F)c(C#N)c3)n2)ccc1C(=O)N(C)C] | 74 |
| 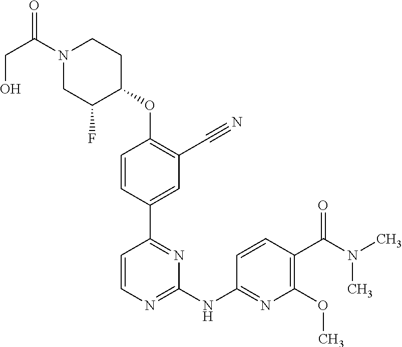 [SMILES: COc1nc(Nc2nccc(-c3ccc(O[C@H]4CCN(C(=O)CO)C[C@H]4F)c(C#N)c3)n2)ccc1C(=O)N(C)C] | 75 |
| 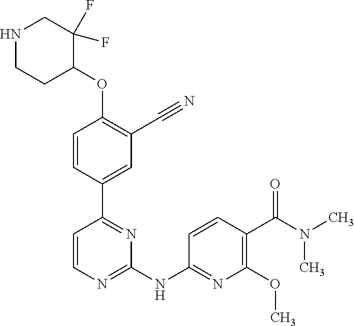 [SMILES: COc1nc(Nc2nccc(-c3ccc(OC4CCNCC4(F)F)c(C#N)c3)n2)ccc1C(=O)N(C)C] | 76 |
| 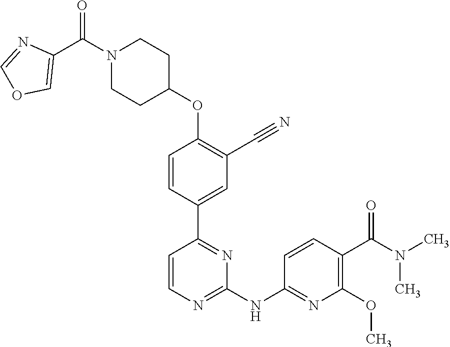 [SMILES: COc1nc(Nc2nccc(-c3ccc(OC4CCN(C(=O)c5cocn5)CC4)c(C#N)c3)n2)ccc1C(=O)N(C)C] | 77 |
| 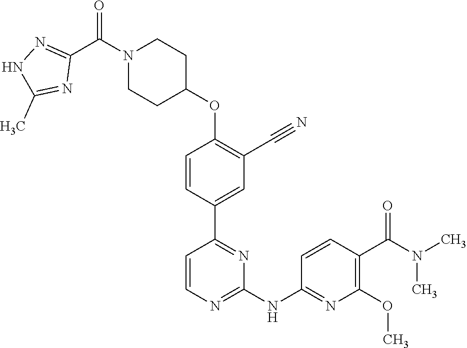 [SMILES: COc1nc(Nc2nccc(-c3ccc(OC4CCN(C(=O)c5n[nH]c(C)n5)CC4)c(C#N)c3)n2)ccc1C(=O)N(C)C] | 78 |
| 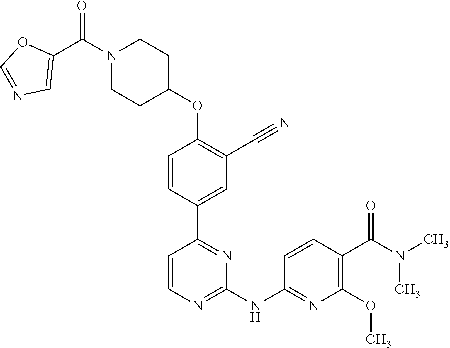 [SMILES: COc1nc(Nc2nccc(-c3ccc(OC4CCN(C(=O)c5cnco5)CC4)c(C#N)c3)n2)ccc1C(=O)N(C)C] | 79 |
| 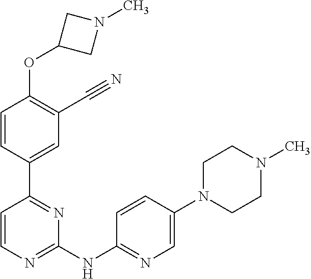 [SMILES: CN1CCN(c2ccc(Nc3nccc(-c4ccc(OC5CN(C)C5)c(C#N)c4)n3)nc2)CC1] | 81 |
| 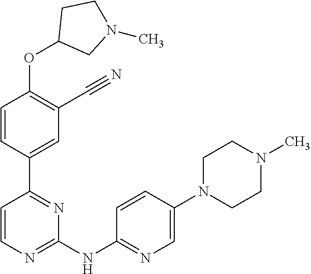 [SMILES: CN1CCN(c2ccc(Nc3nccc(-c4ccc(OC5CCN(C)C5)c(C#N)c4)n3)nc2)CC1] | 82 |
| 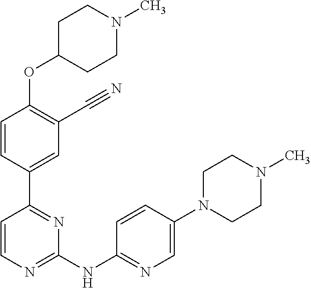 [SMILES: CN1CCC(Oc2ccc(-c3ccnc(Nc4ccc(N5CCN(C)CC5)cn4)n3)cc2C#N)CC1] | 83 |
| 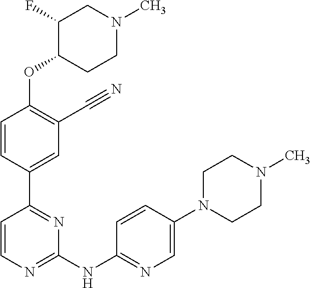 [SMILES: CN1CCN(c2ccc(Nc3nccc(-c4ccc(O[C@H]5CCN(C)C[C@H]5F)c(C#N)c4)n3)nc2)CC1] | 84 |
| 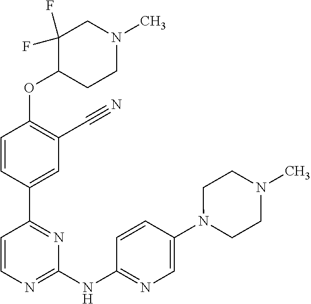 [SMILES: CN1CCN(c2ccc(Nc3nccc(-c4ccc(OC5CCN(C)CC5(F)F)c(C#N)c4)n3)nc2)CC1] | 85 |
| 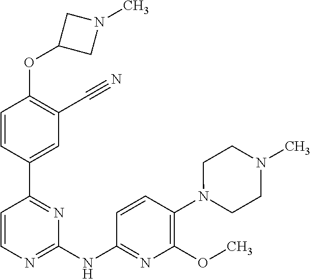 [SMILES: COc1nc(Nc2nccc(-c3ccc(OC4CN(C)C4)c(C#N)c3)n2)ccc1N1CCN(C)CC1] | 86 |
| 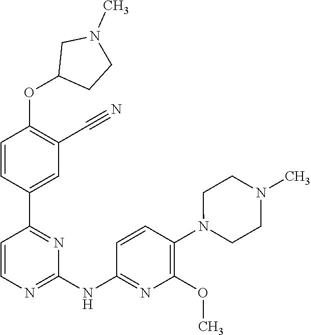 [SMILES: COc1nc(Nc2nccc(-c3ccc(OC4CCN(C)C4)c(C#N)c3)n2)ccc1N1CCN(C)CC1] | 87 |
| 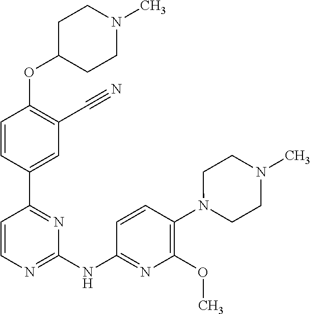 [SMILES: COc1nc(Nc2nccc(-c3ccc(OC4CCN(C)CC4)c(C#N)c3)n2)ccc1N1CCN(C)CC1] | 88 |
| 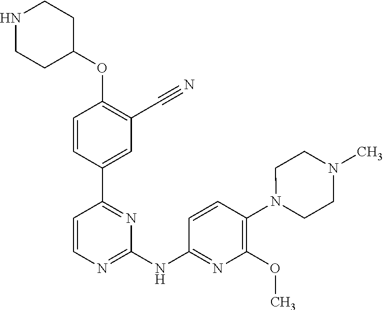 [SMILES: COc1nc(Nc2nccc(-c3ccc(OC4CCNCC4)c(C#N)c3)n2)ccc1N1CCN(C)CC1] | 89 |
| 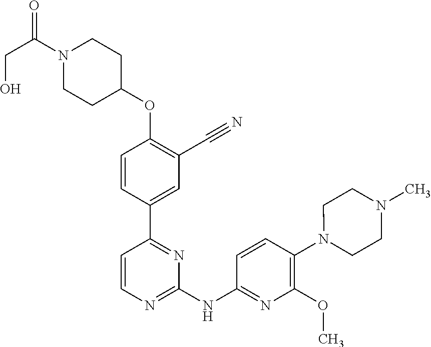 [SMILES: COc1nc(Nc2nccc(-c3ccc(OC4CCN(C(=O)CO)CC4)c(C#N)c3)n2)ccc1N1CCN(C)CC1] | 90 |
| 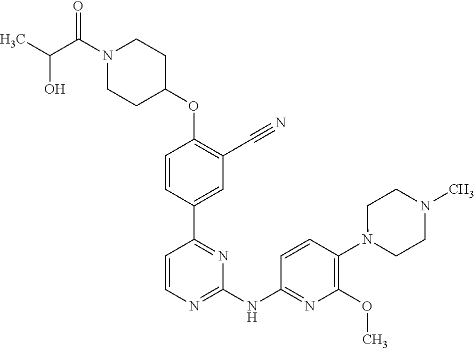 [SMILES: COc1nc(Nc2nccc(-c3ccc(OC4CCN(C(=O)C(C)O)CC4)c(C#N)c3)n2)ccc1N1CCN(C)CC1] | 91 |
| 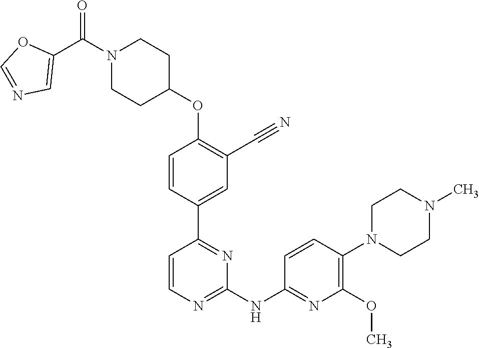 [SMILES: COc1nc(Nc2nccc(-c3ccc(OC4CCN(C(=O)c5cnco5)CC4)c(C#N)c3)n2)ccc1N1CCN(C)CC1] | 92 |
| 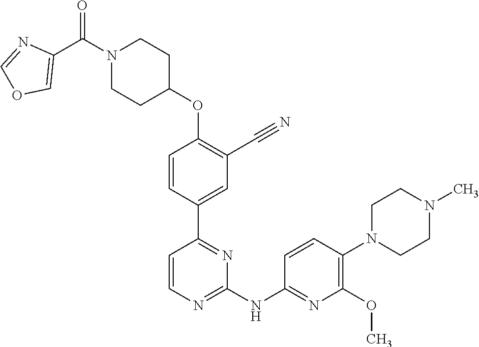 [SMILES: COc1nc(Nc2nccc(-c3ccc(OC4CCN(C(=O)c5cocn5)CC4)c(C#N)c3)n2)ccc1N1CCN(C)CC1] | 93 |
| 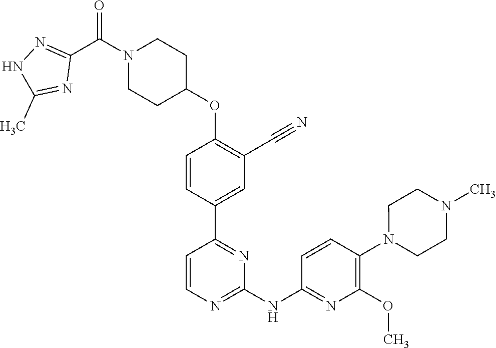 [SMILES: COc1nc(Nc2nccc(-c3ccc(OC4CCN(C(=O)c5n[nH]c(C)n5)CC4)c(C#N)c3)n2)ccc1N1CCN(C)CC1] | 94 |
| 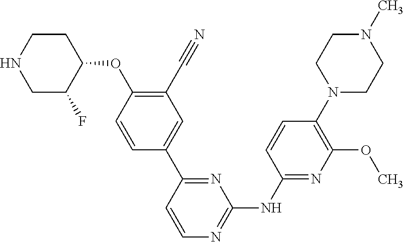 [SMILES: COc1nc(Nc2nccc(-c3ccc(O[C@H]4CCNC[C@H]4F)c(C#N)c3)n2)ccc1N1CCN(C)CC1] | 95 |
| 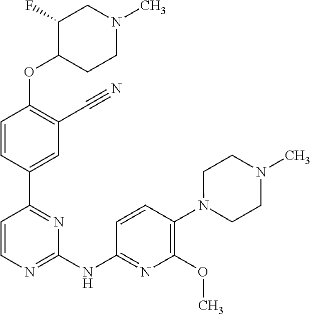 [SMILES: COc1nc(Nc2nccc(-c3ccc(OC4CCN(C)C[C@H]4F)c(C#N)c3)n2)ccc1N1CCN(C)CC1] | 96 |
| 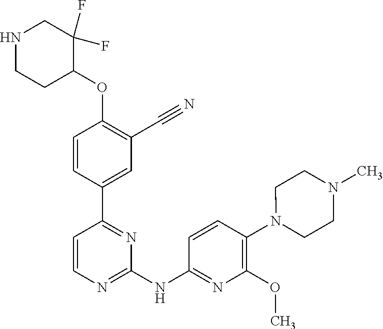 [SMILES: COc1nc(Nc2nccc(-c3ccc(OC4CCNCC4(F)F)c(C#N)c3)n2)ccc1N1CCN(C)CC1] | 97 |
| 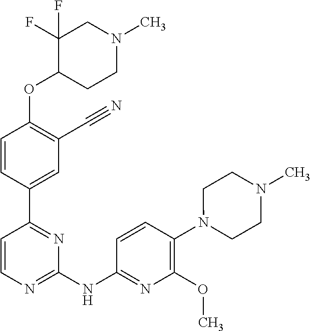 [SMILES: COc1nc(Nc2nccc(-c3ccc(OC4CCN(C)CC4(F)F)c(C#N)c3)n2)ccc1N1CCN(C)CC1] | 98 |
| 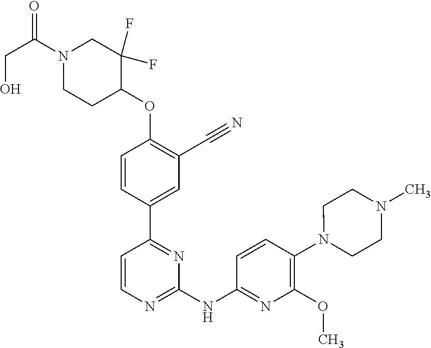 [SMILES: COc1nc(Nc2nccc(-c3ccc(OC4CCN(C(=O)CO)CC4(F)F)c(C#N)c3)n2)ccc1N1CCN(C)CC1] | 99 |
| 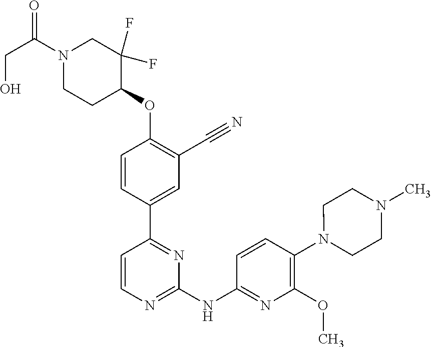 [SMILES: COc1nc(Nc2nccc(-c3ccc(O[C@H]4CCN(C(=O)CO)CC4(F)F)c(C#N)c3)n2)ccc1N1CCN(C)CC1] | 100 |
| 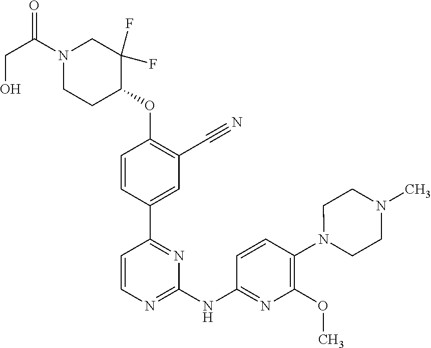 [SMILES: COc1nc(Nc2nccc(-c3ccc(O[C@@H]4CCN(C(=O)CO)CC4(F)F)c(C#N)c3)n2)ccc1N1CCN(C)CC1] | 101 |
| 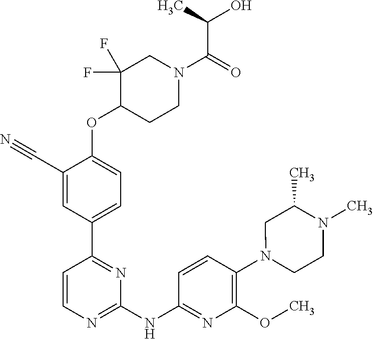 [SMILES: COc1nc(Nc2nccc(-c3ccc(OC4CCN(C(=O)[C@@H](C)O)CC4(F)F)c(C#N)c3)n2)ccc1N1CCN(C)[C@@H](C)C1] | 102 |
| 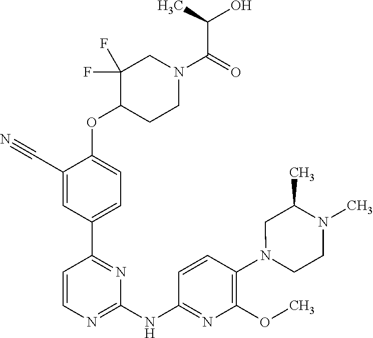 [SMILES: COc1nc(Nc2nccc(-c3ccc(OC4CCN(C(=O)[C@@H](C)O)CC4(F)F)c(C#N)c3)n2)ccc1N1CCN(C)[C@H](C)C1] | 103 |
| 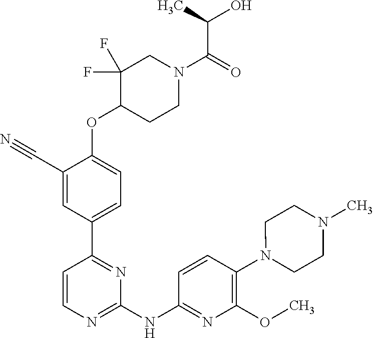 [SMILES: COc1nc(Nc2nccc(-c3ccc(OC4CCN(C(=O)[C@@H](C)O)CC4(F)F)c(C#N)c3)n2)ccc1N1CCN(C)CC1] | 104 |
| 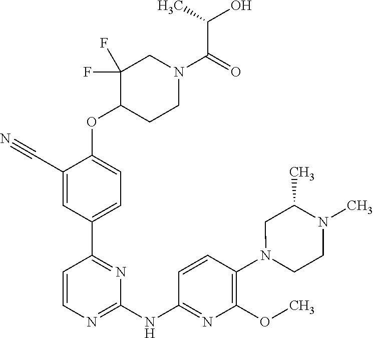 [SMILES: COc1nc(Nc2nccc(-c3ccc(OC4CCN(C(=O)[C@H](C)O)CC4(F)F)c(C#N)c3)n2)ccc1N1CCN(C)[C@@H](C)C1] | 105 |
| 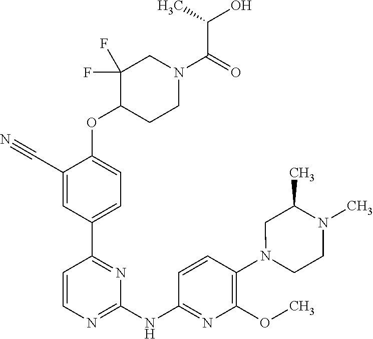 [SMILES: COc1nc(Nc2nccc(-c3ccc(OC4CCN(C(=O)[C@H](C)O)CC4(F)F)c(C#N)c3)n2)ccc1N1CCN(C)[C@H](C)C1] | 106 |
| 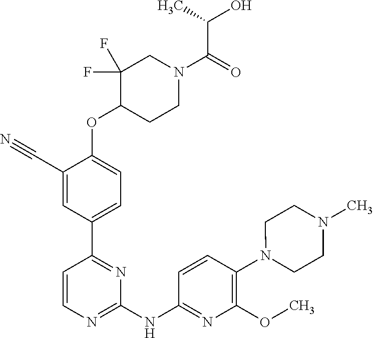 [SMILES: COc1nc(Nc2nccc(-c3ccc(OC4CCN(C(=O)[C@H](C)O)CC4(F)F)c(C#N)c3)n2)ccc1N1CCN(C)CC1] | 107 |
| 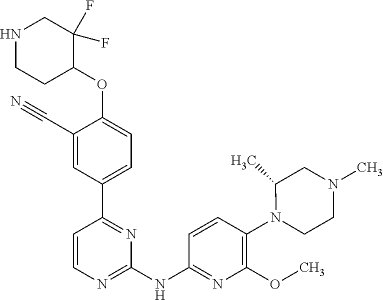 [SMILES: COc1nc(Nc2nccc(-c3ccc(OC4CCNCC4(F)F)c(C#N)c3)n2)ccc1N1CCN(C)C[C@H]1C] | 108 |
| 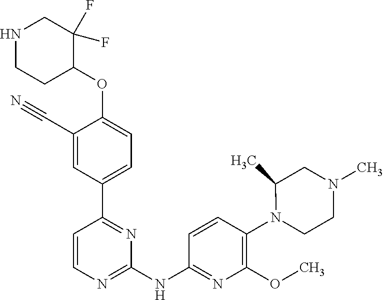 [SMILES: COc1nc(Nc2nccc(-c3ccc(OC4CCNCC4(F)F)c(C#N)c3)n2)ccc1N1CCN(C)C[C@@H]1C] | 109 |
| 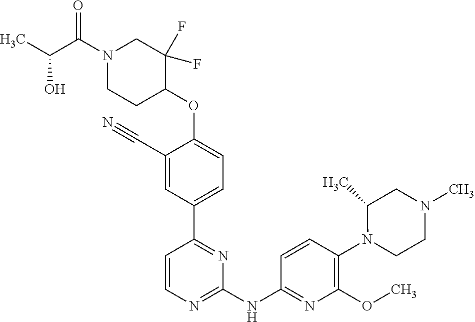 [SMILES: COc1nc(Nc2nccc(-c3ccc(OC4CCN(C(=O)[C@@H](C)O)CC4(F)F)c(C#N)c3)n2)ccc1N1CCN(C)C[C@H]1C] | 110 |
| 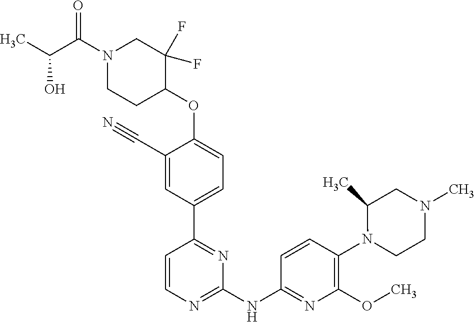 [SMILES: COc1nc(Nc2nccc(-c3ccc(OC4CCN(C(=O)[C@@H](C)O)CC4(F)F)c(C#N)c3)n2)ccc1N1CCN(C)C[C@@H]1C] | 111 |
| 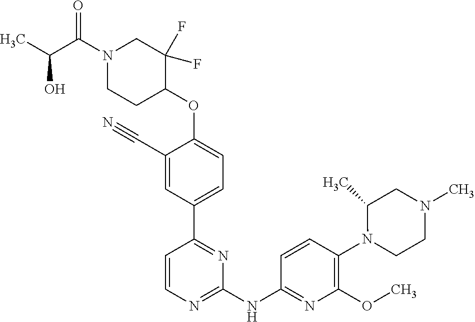 [SMILES: COc1nc(Nc2nccc(-c3ccc(OC4CCN(C(=O)[C@H](C)O)CC4(F)F)c(C#N)c3)n2)ccc1N1CCN(C)C[C@H]1C] | 112 |
| 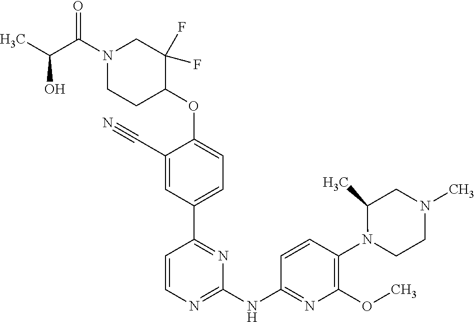 [SMILES: COc1nc(Nc2nccc(-c3ccc(OC4CCN(C(=O)[C@H](C)O)CC4(F)F)c(C#N)c3)n2)ccc1N1CCN(C)C[C@@H]1C] | 113 |
| 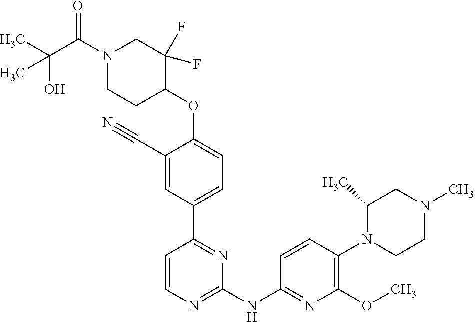 [SMILES: COc1nc(Nc2nccc(-c3ccc(OC4CCN(C(=O)C(C)(C)O)CC4(F)F)c(C#N)c3)n2)ccc1N1CCN(C)C[C@H]1C] | 114 |
| 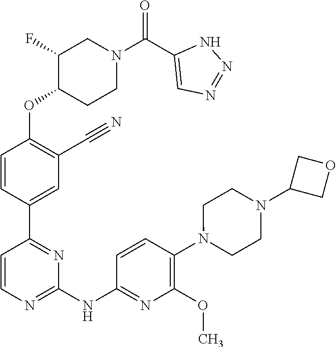 [SMILES: COc1nc(Nc2nccc(-c3ccc(O[C@H]4CCN(C(=O)c5cnn[nH]5)C[C@H]4F)c(C#N)c3)n2)ccc1N1CCN(C2COC2)CC1] | 115 |
| 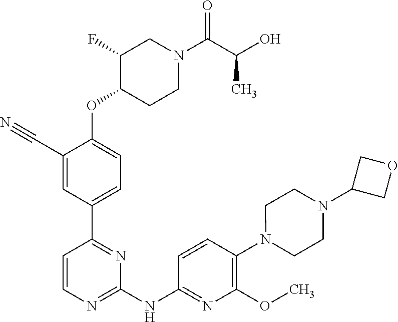 [SMILES: COc1nc(Nc2nccc(-c3ccc(O[C@H]4CCN(C(=O)[C@H](C)O)C[C@H]4F)c(C#N)c3)n2)ccc1N1CCN(C2COC2)CC1] | 116 |
| 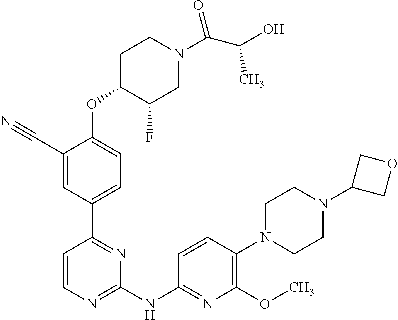 [SMILES: COc1nc(Nc2nccc(-c3ccc(O[C@@H]4CCN(C(=O)[C@@H](C)O)C[C@@H]4F)c(C#N)c3)n2)ccc1N1CCN(C2COC2)CC1] | 117 |
| 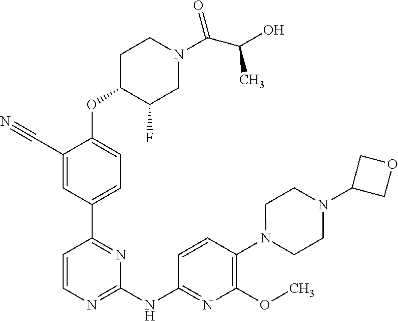 [SMILES: COc1nc(Nc2nccc(-c3ccc(O[C@@H]4CCN(C(=O)[C@H](C)O)C[C@@H]4F)c(C#N)c3)n2)ccc1N1CCN(C2COC2)CC1] | 118 |
| 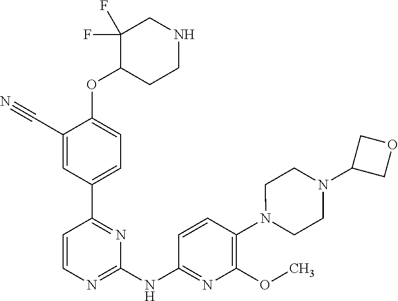 [SMILES: COc1nc(Nc2nccc(-c3ccc(OC4CCNCC4(F)F)c(C#N)c3)n2)ccc1N1CCN(C2COC2)CC1] | 119 |
| 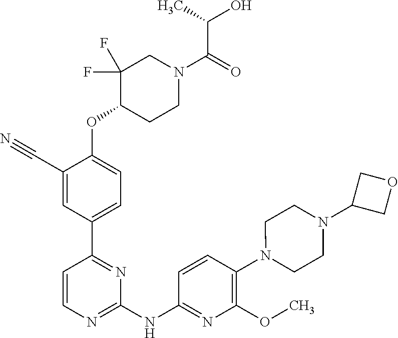 [SMILES: COc1nc(Nc2nccc(-c3ccc(O[C@H]4CCN(C(=O)[C@H](C)O)CC4(F)F)c(C#N)c3)n2)ccc1N1CCN(C2COC2)CC1] | 120 |
| 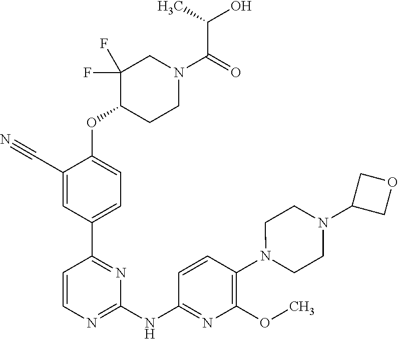 [SMILES: COc1nc(Nc2nccc(-c3ccc(O[C@H]4CCN(C(=O)[C@H](C)O)CC4(F)F)c(C#N)c3)n2)ccc1N1CCN(C2COC2)CC1] | 121 |
| 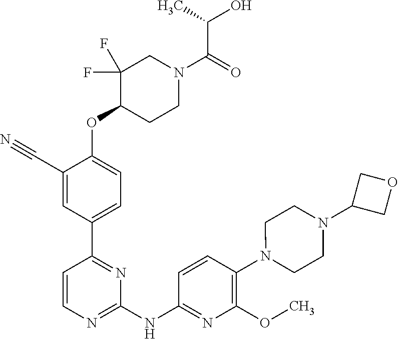 [SMILES: COc1nc(Nc2nccc(-c3ccc(O[C@@H]4CCN(C(=O)[C@H](C)O)CC4(F)F)c(C#N)c3)n2)ccc1N1CCN(C2COC2)CC1] | 122 |
| 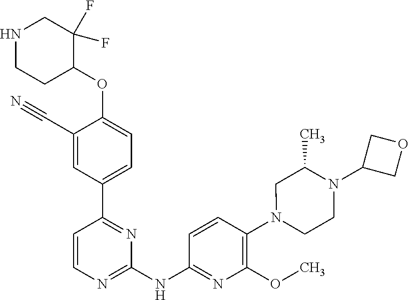 [SMILES: COc1nc(Nc2nccc(-c3ccc(OC4CCNCC4(F)F)c(C#N)c3)n2)ccc1N1CCN(C2COC2)[C@@H](C)C1] | 123 |
| 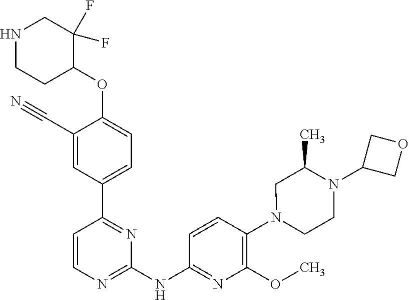 [SMILES: COc1nc(Nc2nccc(-c3ccc(OC4CCNCC4(F)F)c(C#N)c3)n2)ccc1N1CCN(C2COC2)[C@H](C)C1] | 124 |
| 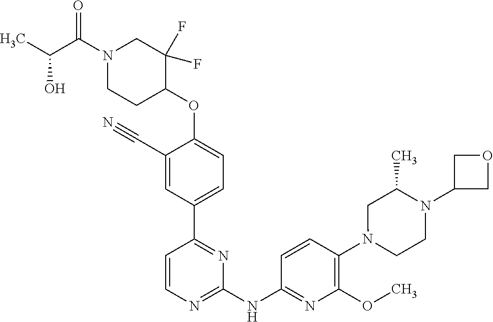 [SMILES: COc1nc(Nc2nccc(-c3ccc(OC4CCN(C(=O)[C@@H](C)O)CC4(F)F)c(C#N)c3)n2)ccc1N1CCN(C2COC2)[C@@H](C)C1] | 125 |
| 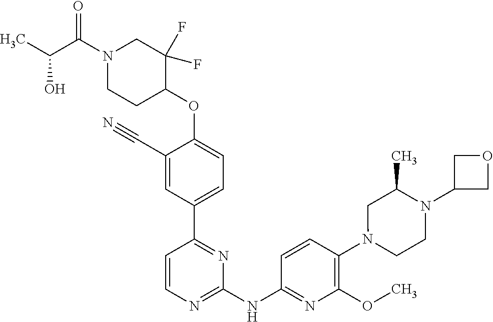 [SMILES: COc1nc(Nc2nccc(-c3ccc(OC4CCN(C(=O)[C@@H](C)O)CC4(F)F)c(C#N)c3)n2)ccc1N1CCN(C2COC2)[C@H](C)C1] | 126 |
| 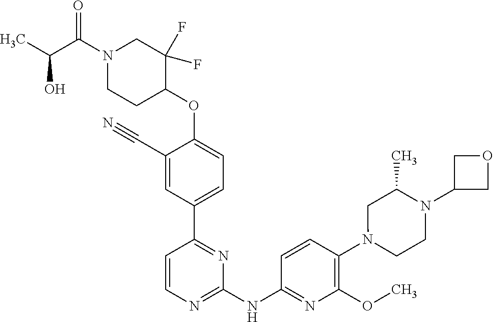 [SMILES: COc1nc(Nc2nccc(-c3ccc(OC4CCN(C(=O)[C@H](C)O)CC4(F)F)c(C#N)c3)n2)ccc1N1CCN(C2COC2)[C@@H](C)C1] | 127 |
| 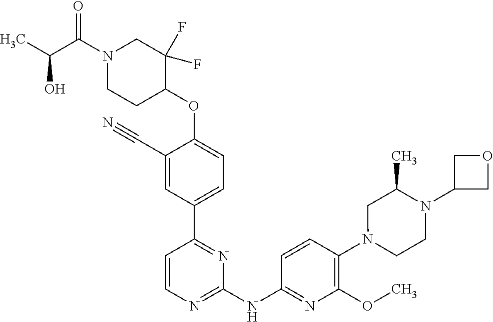 [SMILES: COc1nc(Nc2nccc(-c3ccc(OC4CCN(C(=O)[C@H](C)O)CC4(F)F)c(C#N)c3)n2)ccc1N1CCN(C2COC2)[C@H](C)C1] | 128 |
| 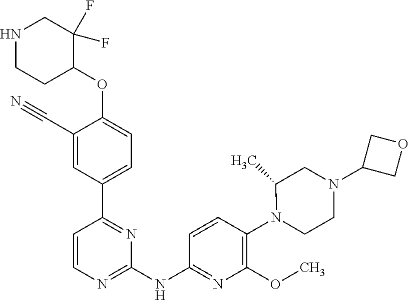 [SMILES: COc1nc(Nc2nccc(-c3ccc(OC4CCNCC4(F)F)c(C#N)c3)n2)ccc1N1CCN(C2COC2)C[C@H]1C] | 129 |
| 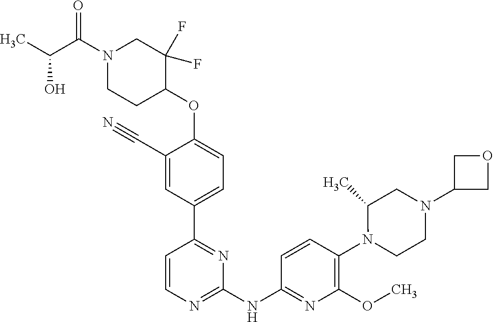 [SMILES: COc1nc(Nc2nccc(-c3ccc(OC4CCN(C(=O)[C@@H](C)O)CC4(F)F)c(C#N)c3)n2)ccc1N1CCN(C2COC2)C[C@H]1C] | 130 |
| 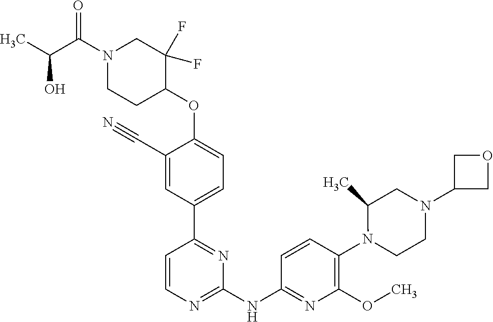 [SMILES: COc1nc(Nc2nccc(-c3ccc(OC4CCN(C(=O)[C@H](C)O)CC4(F)F)c(C#N)c3)n2)ccc1N1CCN(C2COC2)C[C@@H]1C] | 131 |
| 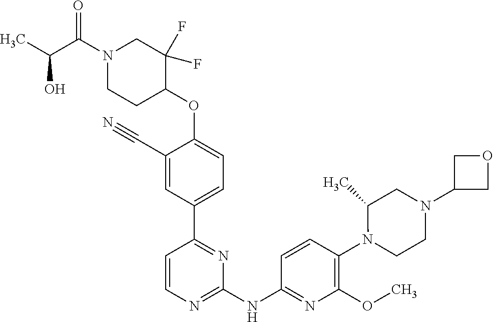 [SMILES: COc1nc(Nc2nccc(-c3ccc(OC4CCN(C(=O)[C@H](C)O)CC4(F)F)c(C#N)c3)n2)ccc1N1CCN(C2COC2)C[C@H]1C] | 132 |
| 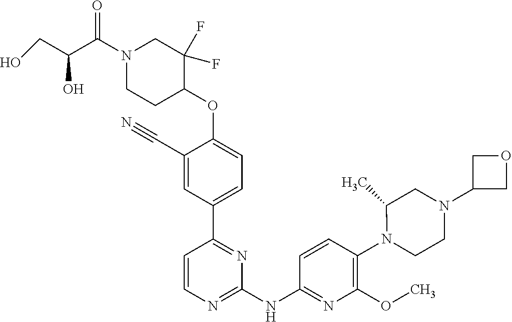 [SMILES: COc1nc(Nc2nccc(-c3ccc(OC4CCN(C(=O)[C@@H](O)CO)CC4(F)F)c(C#N)c3)n2)ccc1N1CCN(C2COC2)C[C@H]1C] | 133 |
| 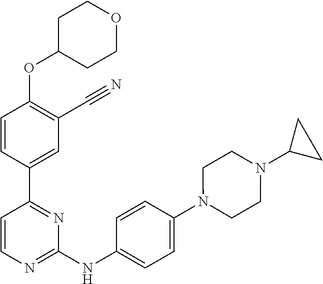 [SMILES: N#Cc1cc(-c2ccnc(Nc3ccc(N4CCN(C5CC5)CC4)cc3)n2)ccc1OC1CCOCC1] | 134 |
| 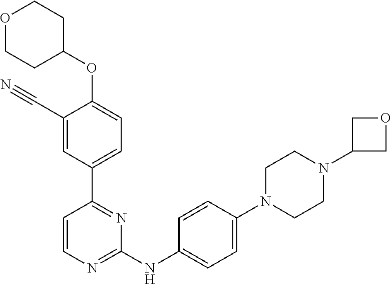 [SMILES: N#Cc1cc(-c2ccnc(Nc3ccc(N4CCN(C5COC5)CC4)cc3)n2)ccc1OC1CCOCC1] | 135 |
| 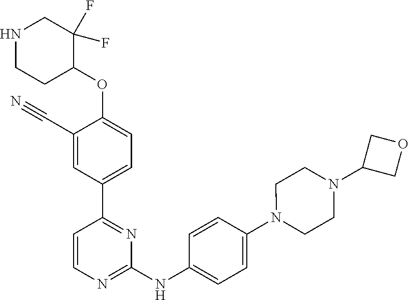 [SMILES: N#Cc1cc(-c2ccnc(Nc3ccc(N4CCN(C5COC5)CC4)cc3)n2)ccc1OC1CCNCC1(F)F] | 136 |
| 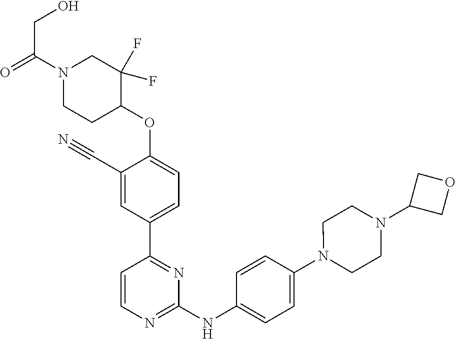 [SMILES: N#Cc1cc(-c2ccnc(Nc3ccc(N4CCN(C5COC5)CC4)cc3)n2)ccc1OC1CCN(C(=O)CO)CC1(F)F] | 137 |
| 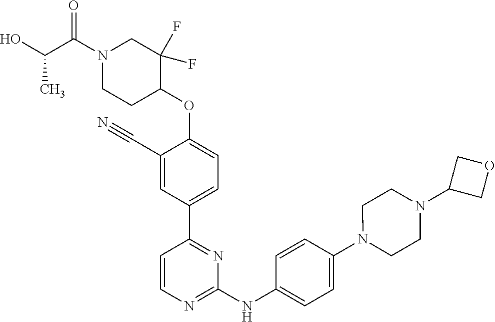 [SMILES: C[C@H](O)C(=O)N1CCC(Oc2ccc(-c3ccnc(Nc4ccc(N5CCN(C6COC6)CC5)cc4)n3)cc2C#N)C(F)(F)C1] | 138 |
| 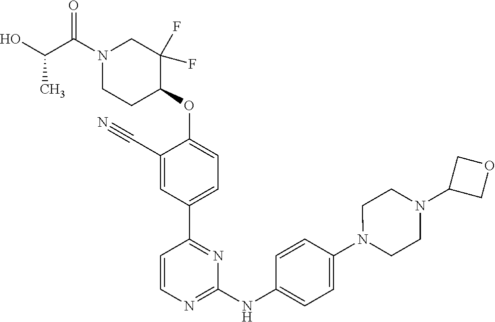 [SMILES: C[C@H](O)C(=O)N1CC[C@H](Oc2ccc(-c3ccnc(Nc4ccc(N5CCN(C6COC6)CC5)cc4)n3)cc2C#N)C(F)(F)C1] | 139 |
| 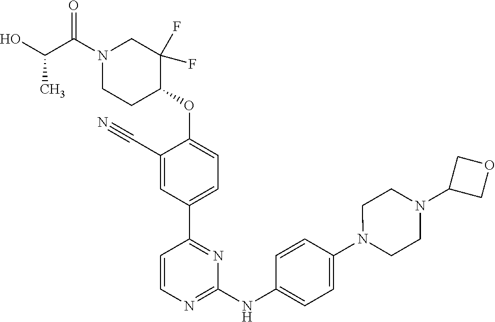 [SMILES: C[C@H](O)C(=O)N1CC[C@@H](Oc2ccc(-c3ccnc(Nc4ccc(N5CCN(C6COC6)CC5)cc4)n3)cc2C#N)C(F)(F)C1] | 140 |
| 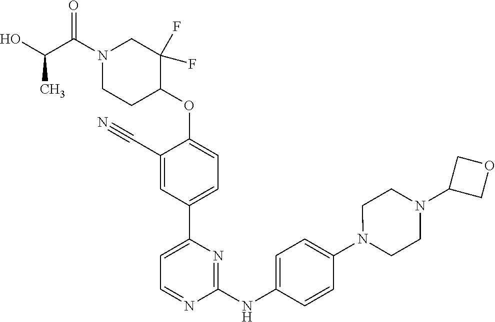 [SMILES: C[C@@H](O)C(=O)N1CCC(Oc2ccc(-c3ccnc(Nc4ccc(N5CCN(C6COC6)CC5)cc4)n3)cc2C#N)C(F)(F)C1] | 141 |
| 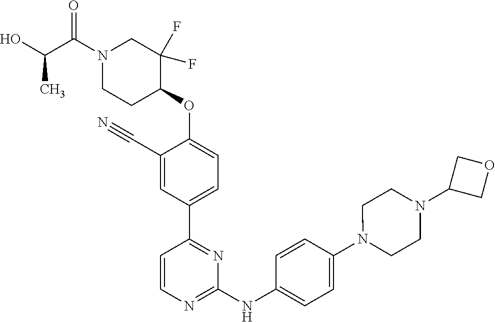 [SMILES: C[C@@H](O)C(=O)N1CC[C@H](Oc2ccc(-c3ccnc(Nc4ccc(N5CCN(C6COC6)CC5)cc4)n3)cc2C#N)C(F)(F)C1] | 142 |
| 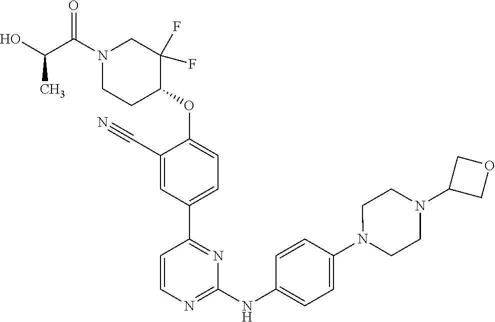 [SMILES: C[C@@H](O)C(=O)N1CC[C@@H](Oc2ccc(-c3ccnc(Nc4ccc(N5CCN(C6COC6)CC5)cc4)n3)cc2C#N)C(F)(F)C1] | 143 |
| 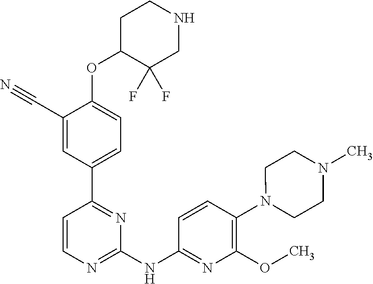 [SMILES: COc1nc(Nc2nccc(-c3ccc(OC4CCNCC4(F)F)c(C#N)c3)n2)ccc1N1CCN(C)CC1] | 144 |
| 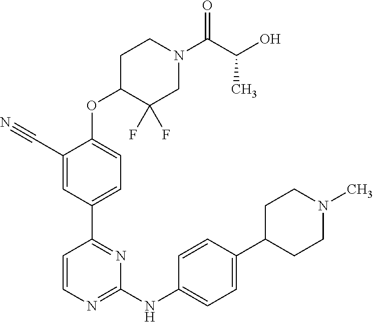 [SMILES: C[C@@H](O)C(=O)N1CCC(Oc2ccc(-c3ccnc(Nc4ccc(C5CCN(C)CC5)cc4)n3)cc2C#N)C(F)(F)C1] | 145 |
| 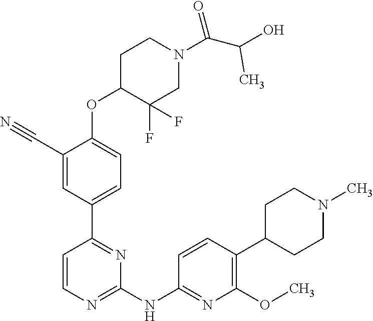 [SMILES: COc1nc(Nc2nccc(-c3ccc(OC4CCN(C(=O)C(C)O)CC4(F)F)c(C#N)c3)n2)ccc1C1CCN(C)CC1] | 146 |
| 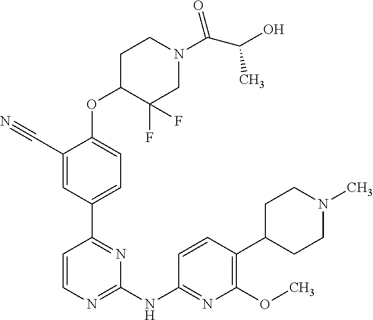 [SMILES: COc1nc(Nc2nccc(-c3ccc(OC4CCN(C(=O)[C@@H](C)O)CC4(F)F)c(C#N)c3)n2)ccc1C1CCN(C)CC1] | 147 |
| 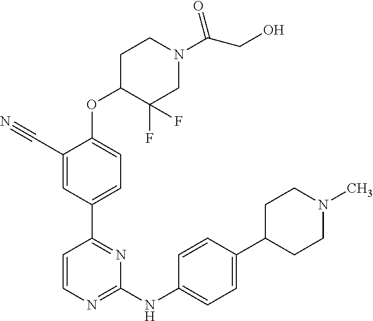 [SMILES: CN1CCC(c2ccc(Nc3nccc(-c4ccc(OC5CCN(C(=O)CO)CC5(F)F)c(C#N)c4)n3)cc2)CC1] | 148 |
| 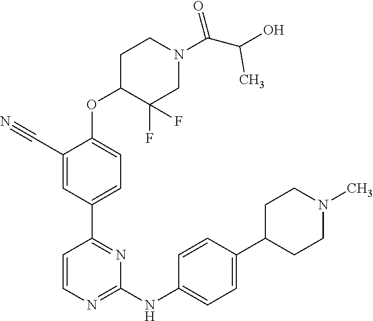 [SMILES: CC(O)C(=O)N1CCC(Oc2ccc(-c3ccnc(Nc4ccc(C5CCN(C)CC5)cc4)n3)cc2C#N)C(F)(F)C1] | 149 |
| 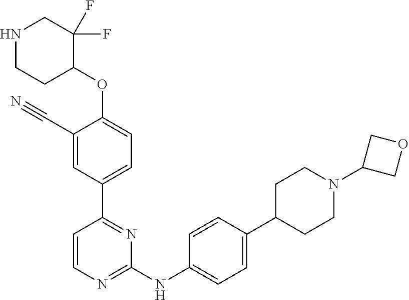 [SMILES: N#Cc1cc(-c2ccnc(Nc3ccc(C4CCN(C5COC5)CC4)cc3)n2)ccc1OC1CCNCC1(F)F] | 150 |
| 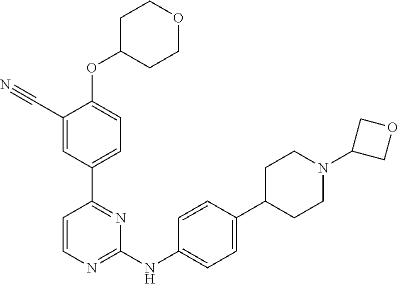 [SMILES: N#Cc1cc(-c2ccnc(Nc3ccc(C4CCN(C5COC5)CC4)cc3)n2)ccc1OC1CCOCC1] | 151 |
| 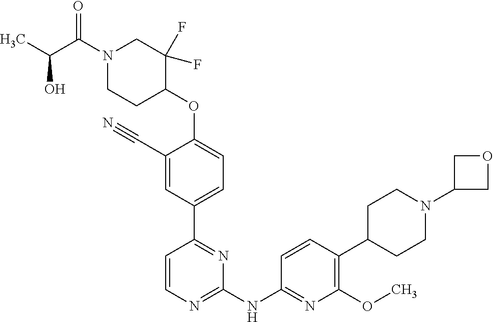 [SMILES: COc1nc(Nc2nccc(-c3ccc(OC4CCN(C(=O)[C@H](C)O)CC4(F)F)c(C#N)c3)n2)ccc1C1CCN(C2COC2)CC1] | 152 |
| 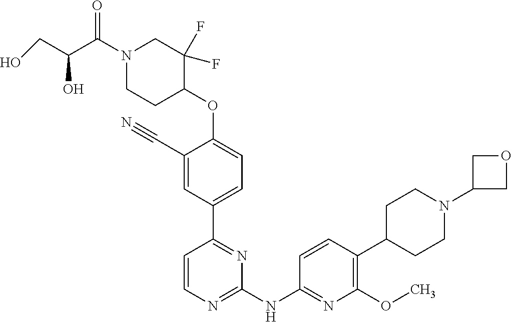 [SMILES: COc1nc(Nc2nccc(-c3ccc(OC4CCN(C(=O)[C@@H](O)CO)CC4(F)F)c(C#N)c3)n2)ccc1C1CCN(C2COC2)CC1] | 153 |
| 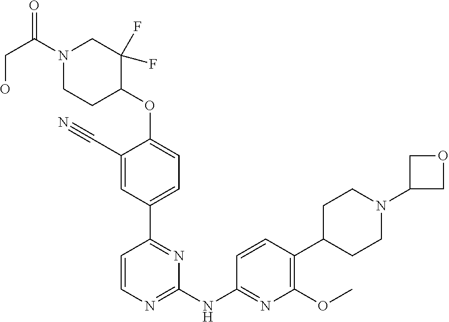 [SMILES: COc1nc(Nc2nccc(-c3ccc(OC4CCN(C(=O)CO)CC4(F)F)c(C#N)c3)n2)ccc1C1CCN(C2COC2)CC1] | 154 |
| 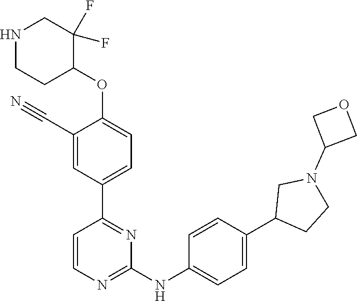 [SMILES: N#Cc1cc(-c2ccnc(Nc3ccc(C4CCN(C5COC5)C4)cc3)n2)ccc1OC1CCNCC1(F)F] | 155 |
| 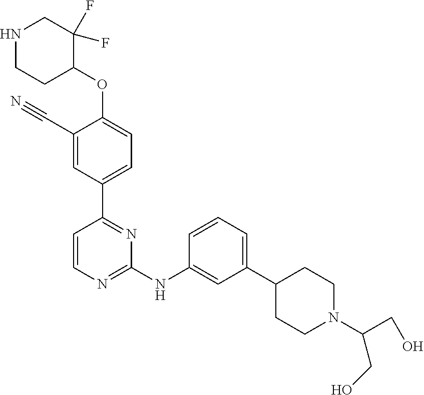 [SMILES: N#Cc1cc(-c2ccnc(Nc3cccc(C4CCN(C(CO)CO)CC4)c3)n2)ccc1OC1CCNCC1(F)F] | 156 |
| 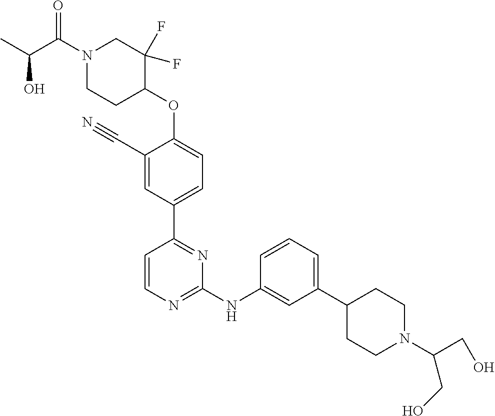 [SMILES: C[C@H](O)C(=O)N1CCC(Oc2ccc(-c3ccnc(Nc4cccc(C5CCN(C(CO)CO)CC5)c4)n3)cc2C#N)C(F)(F)C1] | 157 |
| 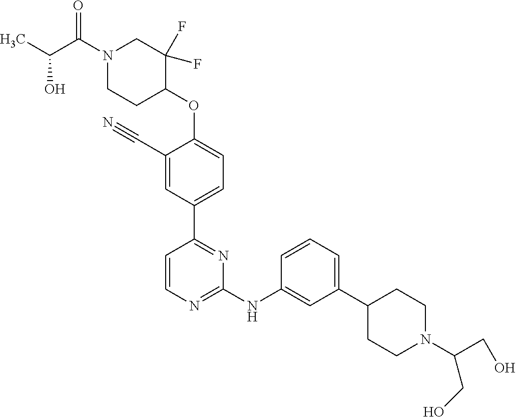 [SMILES: C[C@@H](O)C(=O)N1CCC(Oc2ccc(-c3ccnc(Nc4cccc(C5CCN(C(CO)CO)CC5)c4)n3)cc2C#N)C(F)(F)C1] | 158 |
| 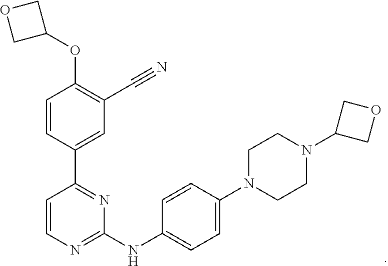 [SMILES: N#Cc1cc(-c2ccnc(Nc3ccc(N4CCN(C5COC5)CC4)cc3)n2)ccc1OC1COC1] | 159 |

In some embodiments, the present invention provides a compound selected from those depicted above, or a pharmaceutically acceptable salt thereof.

Various structural depictions may show a heteroatom without an attached group, radical, charge, or counterion. Those of ordinary skill in the art are aware that such depictions are meant to indicate that the heteroatom is attached to hydrogen (e.g.,

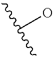 [SMILES: *O]

is understood to be

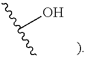 [SMILES: *O]

In certain embodiments, the compounds of the invention were synthesized in accordance with the schemes provided in the Examples below.

4. Uses. Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit TBK and IKKε, or a mutant thereof, in a biological sample or in a patient. In certain embodiments, the amount of compound in compositions of this invention is such that is effective to measurably inhibit TBK and IKKε, or a mutant thereof, in a biological sample or in a patient. In certain embodiments, a composition of this invention is formulated for administration to a patient in need of such composition.

The term “patient” or “subject”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this aremineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

The present invention furthermore relates to a method for treating a subject suffering from a TBK or IKKε related disorder, comprising administering to said subject an effective amount of a compound of formula I or any formulae presented herein.

The present invention preferably relates to a method, wherein the TBK or IKKε associated disorder is an autoimmune disorder or condition associated with an overactive immune response or cancer. The present invention furthermore relates to a method of treating a subject suffering from an immunoregulatory abnormality, comprising administering to said subject a compound of formula (I), and related formulae in an amount that is effective for treating said immunoregulatory abnormality.

The present invention preferably relates to a method wherein the immunoregulatory abnormality is an autoimmune or chronic inflammatory disease selected from the group consisting of: allergic diseases, amyotrophic lateral sclerosis (ALS), systemic lupus erythematosus, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy and asthma.

The present invention furthermore relates to a method wherein the immunoregulatory abnormality is bone marrow or organ transplant rejection or graft-versus-host disease.

The present invention furthermore relates to a method wherein the immunoregulatory abnormality is selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, heipetic keratitis, conical cornea, dystrophia epithelialis comeae, comeal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, alleigic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, parkison diseases, trauma, and chronic bacterial infection.

In certain embodiments, disorders associated with TBK or IKKε are selected from Rheumatoid Arthritis, Psoriatic arthritis, Osteoarthritis, Systemic Lupus Erythematosus, Lupus nephritis, Ankylosing Spondylitis, Osteoporosis, Systemic sclerosis, Multiple Sclerosis, Psoriasis, Type I diabetes, Type II diabetes, Inflammatory Bowel Disease (Crohn's Disease and Ulcerative Colitis), Hyperimmunoglobulinemia D and periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, DIRA (Deficiency of IL-1 Receptor Antagonist), Alzheimer's disease, Parkinson's disease, and Cancer.

In certain embodiments, disorders associated with TBK or IKKε are selected from cancer, septic shock, Primary open Angle Glaucoma (POAG), hyperplasia, rheumatoid arthritis, psoriasis, artherosclerosis, retinopathy, osteoarthritis, endometriosis, chronic inflammation, and/or neurodegenerative diseases such as Alzheimers disease.

In certain embodiments, the cancer is selected from carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer.

In certain embodiments, the cancer is brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, Kaposi's sarcoma, or any other type of solid or liquid tumors. In some embodiments, the cancer is metastatic cancer. In some embodiments, the cancer is colorectal cancer. In some embodiments, the cancer is colon cancer.

In certain aspects, the invention relates to the compounds of the invention for the use for the treatment of a disease or disorder described herein.

In certain aspects, the invention relates to the use of compounds of formula I, or any formulae presented herein, for the preparation of a medicament for the treatment or a disease or disorder described herein.

In various embodiments, compounds of formula (I), and related formulae exhibit a IC50 for the binding to TBK and/or IKKε of less than about 5 μM, preferably less than about 1 μM, preferably less than about 100 nM, preferably less than about 10 nM.

The method of the invention can be performed either in-vitro or in-vivo. The susceptibility of a particular cell to treatment with the compounds according to the invention can be particularly determined by in-vitro tests, whether in the course of research or clinical application. Typically, a culture of the cell is combined with a compound according to the invention at various concentrations for a period of time which is sufficient to allow the active agents to inhibit TBK and/or IKKε activity, usually between about one hour and one week. In-vitro treatment can be carried out using cultivated cells from a biopsy sample or cell line.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

For identification of a signal transduction pathway and for detection of interactions between various signal transduction pathways, various scientists have developed suitable models or model systems, for example cell culture models and models of transgenic animals. For the determination of certain stages in the signal transduction cascade, interacting compounds can be utilized in order to modulate the signal. The compounds according to the invention can also be used as reagents for testing TBK and/or IKKε-dependent signal transduction pathways in animals and/or cell culture models or in the clinical diseases mentioned in this application.

Moreover, the subsequent teaching of the present specification concerning the use of the compounds according to formula (I) and its derivatives for the production of a medicament for the prophylactic or therapeutic treatment and/or monitoring is considered as valid and applicable without restrictions to the use of the compound for the inhibition of TBK and/or IKKε activity if expedient.

The invention also relates to the use of compounds according to formula (I) and/or physiologically acceptable salts thereof for the prophylactic or therapeutic treatment and/or monitoring of diseases that are caused, mediated and/or propagated by TBK and/or IKKε activity. Furthermore, the invention relates to the use of compounds according to formula (I) and/or physiologically acceptable salts thereof for the production of a medicament for the prophylactic or therapeutic treatment and/or monitoring of diseases that are caused, mediated and/or propagated by TBK and/or IKKε activity. In certain embodiments, the invention provides the use of a compound according to formula I or physiologically acceptable salts thereof, for the production of a medicament for the prophylactic or therapeutic treatment of a TBK and/or IKKε-mediated disorder.

Compounds of formula (I) and/or a physiologically acceptable salt thereof can furthermore be employed as intermediate for the preparation of further medicament active ingredients. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

The compounds of formula (I) according to the invention can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease completely. Either the identical compound or different compounds can be applied. The methods of the invention can also be used to reduce the likelihood of developing a disorder or even prevent the initiation of disorders associated with TBK and/or IKKε activity in advance or to treat the arising and continuing symptoms.

In the meaning of the invention, prophylactic treatment is advisable if the subject possesses any preconditions for the aforementioned physiological or pathological conditions, such as a familial disposition, a genetic defect, or a previously incurred disease.

The invention furthermore relates to a medicament comprising at least one compound according to the invention and/or pharmaceutically usable derivatives, salts, solvates and stereoisomers thereof, including mixtures thereof in all ratios. In certain embodiments, the invention relates to a medicament comprising at least one compound according to the invention and/or physiologically acceptable salts thereof.

A “medicament” in the meaning of the invention is any agent in the field of medicine, which comprises one or more compounds of formula (I) or preparations thereof (e.g. a pharmaceutical composition or pharmaceutical formulation) and can be used in prophylaxis, therapy, follow-up or aftercare of patients who suffer from diseases, which are associated with TBK and/or IKKε activity, in such a way that a pathogenic modification of their overall condition or of the condition of particular regions of the organism could establish at least temporarily.

In various embodiments, the active ingredient may be administered alone or in combination with other treatments. A synergistic effect may be achieved by using more than one compound in the pharmaceutical composition, i.e. the compound of formula (I) is combined with at least another agent as active ingredient, which is either another compound of formula (I) or a compound of different structural scaffold. The active ingredients can be used either simultaneously or sequentially.

Included herein are methods of treatment in which at least one chemical entity provided herein is administered in combination with an anti-inflammatory agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants and methotrexate.

Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-monoclonal antibody (such as eculizumab or pexelizumab), a TNF antagonist, such as entanercept, or infliximab, which is an anti-TNF alpha monoclonal antibody.

Still other embodiments of the invention pertain to combinations in which at least one active agent is an immunosuppressant compound such as an immunosuppressant compound chosen from methotrexate, leflunomide, cyclosporine, tacrolimus, azathioprine, and mycophenolate mofetil.

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined above may be applied as a monotherapy or may involve, in addition to the herein disclosed compounds of formula I, conventional surgery or radiotherapy or medicinal therapy. Such medicinal therapy, e.g. a chemotherapy or a targeted therapy, may include one or more, but preferably one, of the following anti-tumor agents:

Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine^{1,3};
Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur, doxifluridine, elacytarabine, raltitrexed, sapacitabine, tegafur^{2,3}, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide^{1,3};
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴;
Photosensitizers: such as methoxsalen³; porfimer sodium, talaporfin, temoporfin;
Antibodies: such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3}; catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};
Cytokines: such as aldesleukin, interferon alfa², interferon alfa2a³, interferon alfa2b^{2,3}; celmoleukin, tasonermin, teceleukin, oprelvekin¹³, recombinant interferon beta-1a⁴;
Drug Conjugates: such as denileukin diftitox, ibritumomab tiuxetan, iobenguane I123, prednimustine, trastuzumab emtansine, estramustine, gemtuzumab, ozogamicin, aflibercept; cintredekin besudotox, edotreotide, inotuzumab ozogamicin, naptumomab estafenatox, oportuzumab monatox, technetium (99mTc) arcitumomab^{1,3}, vintafolide^{1,3};
Vaccines: such as sipuleucel³; vitespen³, emepepimut-S³, oncoVAX⁴, rindopepimut³, troVax⁴, MGN-1601⁴, MGN-1703⁴; and
Miscellaneous: alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴.
(¹Prop. INN (Proposed International Nonproprietary Name);²Rec. INN (Recommended International Nonproprietary Names);³ USAN (United States Adopted Name);⁴ no INN).

In another aspect, the invention provides for a kit consisting of separate packs of an effective amount of a compound according to the invention and/or pharmaceutically acceptable salts, derivatives, solvates and stereoisomers thereof, including mixtures thereof in all ratios, and optionally, an effective amount of a further active ingredient. The kit comprises suitable containers, such as boxes, individual bottles, bags or ampoules. The kit may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further active ingredient in dissolved or lyophilized form.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder, the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracistemally, intravaginally, intraperitoneally, topically (as by powders, ointments, or drops), bucally, as an oral or nasal spray, or the like, depending on the severity of the infection being treated. In certain embodiments, the compounds of the invention are administered orally or parenterally at dosage levels of about 0.01 mg/kg to about 100 mg/kg and preferably from about 1 mg/kg to about 50 mg/kg, of subject body weight per day, one or more times a day, to obtain the desired therapeutic effect.

In certain embodiments, a therapeutically effective amount of a compound of the formula (I), and related formulae and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

In certain embodiments, the pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Liquid dosage forms for oral administration include, but are not limited to, pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions are formulated according to the known art using suitable dispersing or wetting agents and suspending agents. The sterile injectable preparation are also a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This is accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also optionally comprises buffering agents.

Solid compositions of a similar type are also employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type are also employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting TBK and/or IKKε activity in a biological sample comprising the step of contacting said biological sample with a compound of this invention, or a composition comprising said compound.

According to another embodiment, the invention relates to a method of inhibiting TBK and/or IKKε, or a mutant thereof, activity in a biological sample in a positive manner, comprising the step of contacting said biological sample with a compound of this invention, or a composition comprising said compound.

The compounds of the invention are useful in-vitro as unique tools for understanding the biological role of TBK and/or IKKε, including the evaluation of the many factors thought to influence, and be influenced by, the production of TBK and/or IKKε and the interaction of TBK and/or IKKε. The present compounds are also useful in the development of other compounds that interact with TBK and/or IKKε since the present compounds provide important structure-activity relationship (SAR) information that facilitate that development. Compounds of the present invention that bind to TBK and/or IKKε can be used as reagents for detecting TBK and/or IKKε in living cells, fixed cells, in biological fluids, in tissue homogenates, in purified, natural biological materials, etc. For example, by labeling such compounds, one can identify cells expressing TBK and/or IKKε. In addition, based on their ability to bind TBK and/or IKKε, compounds of the present invention can be used in in-situ staining, FACS (fluorescence-activated cell sorting), sodium dodecyl sulfate polyacrylamide gel electrophoresis (SDS-PAGE), ELISA (enzyme-linked immunoadsoiptive assay), etc., enzyme purification, or in purifying cells expressing TBK and/or IKKε inside permeabilized cells. The compounds of the invention can also be utilized as commercial research reagents for various medical research and diagnostic uses. Such uses can include but are not limited to: use as a calibration standard for quantifying the activities of candidate TBK and/or IKKε inhibitors in a variety of functional assays; use as blocking reagents in random compound screening, i.e. in looking for new families of TBK and/or IKKε ligands, the compounds can be used to block recovery of the presently claimed TBK and/or IKKε compounds; use in the co-crystallization with TBK and/or IKKε enzyme, i.e. the compounds of the present invention will allow formation of crystals of the compound bound to TBK and/or IKKε, enabling the determination of enzyme/compound structure by x-ray crystallography; other research and diagnostic applications, wherein TBK and/or IKKε is preferably activated or such activation is conveniently calibrated against a known quantity of an TBK and/or IKKε inhibitor, etc.; use in assays as probes for determining the expression of TBK and/or IKKε in cells; and developing assays for detecting compounds which bind to the same site as the TBK and/or IKKε binding ligands.

The compounds of the invention can be applied either themselves and/or in combination with physical measurements for diagnostics of treatment effectiveness. Pharmaceutical compositions containing said compounds and the use of said compounds to treat TBK and/or IKKε-mediated conditions is a promising, novel approach for a broad spectrum of therapies causing a direct and immediate improvement in the state of health, whether in human or in animal. The orally bioavailable and active new chemical entities of the invention improve convenience for patients and compliance for physicians.

The compounds of formula (I), their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Modulation of TBK and/or IKKε, or a mutant thereof, activity in a biological sample is useful for a variety of purposes that are known to one of skill in the art. Examples of such purposes include, but are not limited to, blood transfusion, organ transplantation, biological specimen storage, and biological assays.

### EXEMPLIFICATION

### General Conditions and Analytical Methods

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

The symbols and conventions used in the following descriptions of processes, schemes, and examples are consistent with those used in the contemporary scientific literature, for example, the Journal of the American Chemical Society or the Journal of Biological Chemistry.

Unless otherwise indicated, all temperatures are expressed in ° C. (degrees Centigrade).

All reactions were conducted at room temperature unless otherwise noted. All compounds of the present invention were synthesized by processes developed by the inventors.

Compound numbers utilized in the Examples below correspond to compound numbers set forth supra.

In general, the compounds according to Formula (I) and related formulae of this invention can be prepared from readily available starting materials. If such starting materials are not commercially available, they may be prepared by standard synthetic techniques. In general, the synthesis pathways for any individual compound of Formula (I) and related formulae will depend on the specific substituents of each molecule, such factors being appreciated by those of ordinary skilled in the art. The following general methods and procedures described hereinafter in the examples may be employed to prepare compounds of Formula (I) and related formulae. Reaction conditions depicted in the following schemes, such as temperatures, solvents, or co-reagents, are given as examples only and are not restrictive. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures. For all the protection and deprotection methods, see Philip J. Kocienski, in “Protecting Groups”, Geoig Thieme Verlag Stuttgart, New York, 1994 and, Theodora W. Greene and Peter G. M. Wuts in “Protective Groups in Organic Synthesis”, Wiley Interscience, 3^{rd} Edition 1999.

All solvents used were commercially available and were used without further purification. Reactions were typically run using anhydrous solvents under an inert atmosphere of nitrogen. Flash column chromatography was generally carried out using Silica gel 60 (0.035-0.070 mm particle size).

All NMR experiments were recorded either on Bruker Mercury Plus 400 NMR Spectrometer equipped with a Bruker 400 BBFO probe at 400 MHz for proton NMR or on Bruker Mercury Plus 300 NMR Spectrometer equipped with a Bruker 300 BBFO probe at 300 MHz for proton NMR. All deuterated solvents contained typically 0.03% to 0.05% v/v tetramethylsilane, which was used as the reference signal (set at δ 0.00 for both ¹H and ¹³C).

LC-MS analyses were performed on a SHIMADZU LC-MS machine consisting of an UFLC 20-AD system and LCMS 2020 MS detector. The column used was a Shim-pack XR-ODS, 2.2 μm, 3.0×50 mm. A linear gradient was applied, starting at 95% A (A: 0.05% TFA in water) and ending at 100% B (B: 0.05% TFA in acetonitrile) over 2.2 min with a total run time of 3.6 min. The column temperature was at 40° C. with the flow rate at 1.0 mL/min. The Diode Array detector was scanned from 200-400 nm. The mass spectrometer was equipped with an electro spray ion source (ES) operated in a positive or negative mode. The mass spectrometer was scanned between m/z 90-900 with a scan time of 0.6 s.

BPD is the abbreviation for 4,4,5,5-tetramethyl-2-(tetramethyl-1,3,2-dioxaborolan-2-yl)-1,3,2-dioxaborolane.

### Example 1: 6-([4-[3-cyano-4-(oxan-4-yloxy)phenyl]pyrimidin-2-yl]amino)-2-methoxy-N,N-dimethylpyridine-3-carboxamide (1)

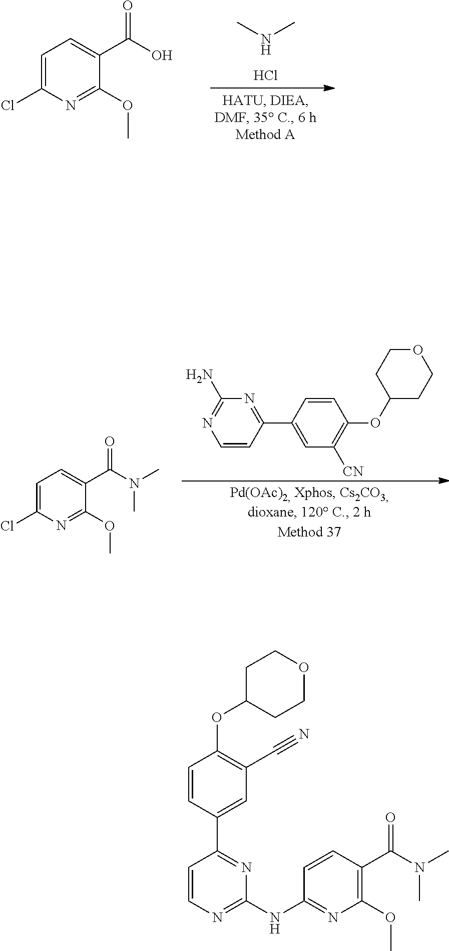

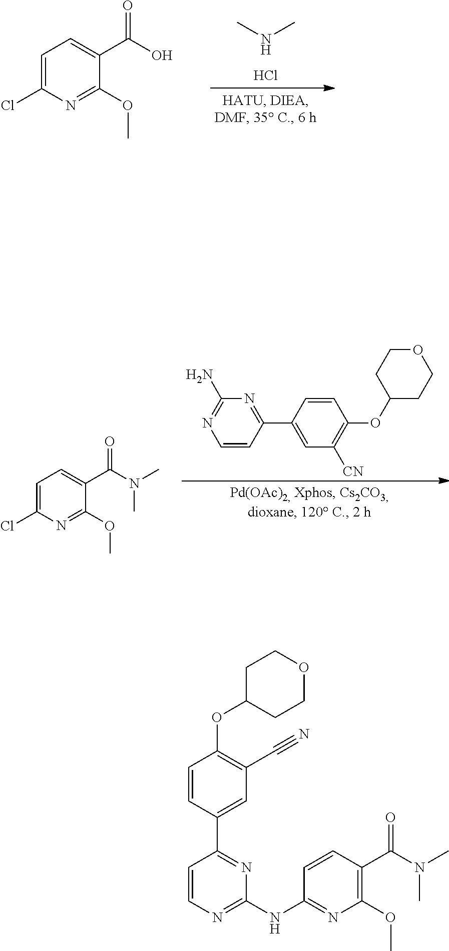

### Method A

6-chloro-2-methoxy-N,N-dimethylpyridine-3-carboxamide: To a solution of 6-chloro-2-methoxypyridine-3-carboxylic acid (190 mg, 1.01 mmol) in N,N-dimethylformamide (2 mL) was added HATU (722 mg, 1.90 mmol), dimethylamine hydrochloride (165 mg, 2.03 mmol) and DIEA (614 mg, 4.75 mmol) at room temperature. The resulting mixture was stirred for 6 h at 35° C. When the reaction was done, the reaction mixture was diluted with H₂O (20 mL) and extracted with ethyl acetate (50 mL×3). The organic phases were combined, washed with brine and dried over Na₂SO₄. The solvent was removed under reduced pressure and the residue was purified by flash chromatography eluting with MeOH in EtOAc (0% to 10% gradient) to yield 6-chloro-2-methoxy-N,N-dimethylpyridine-3-carboxamide as an yellow oil (129 mg, 59%). MS: m/z=214.9 [M+H]⁺.

### Method 37

6-([4-[3-cyano-4-(oxan-4-yloxy)phenyl]pyrimidin-2-yl]amino)-2-methoxy-N,N-dimethylpyridine-3-carboxamide: To a solution of 5-(2-aminopyrimidin-4-yl)-2-(oxan-4-yloxy)benzonitrile (78 mg, 0.26 mmol) in dioxane (6 mL) was added 6-chloro-2-methoxy-N,N-dimethylpyridine-3-carboxamide (62 mg, 0.29 mmol), Pd(OAc)₂ (38 mg, 0.17 mmol), Xphos (76 mg, 0.16 mmol) and Cs₂CO₃ (238 mg, 0.73 mmol) at room temperature. The resulting mixture was stirred for 2 h at 120° C. When the reaction was done, the resulting mixture was concentrated under reduced pressure and the residue was purified by prep-HPLC to obtain 6-([4-[3-cyano-4-(oxan-4-yloxy)phenyl]pyrimidin-2-yl]amino)-2-methoxy-N,N-dimethylpyridine-3-carboxamide as an yellow solid (27 mg, 22%). HPLC: 97.0% purity, RT=1.28 min. MS: m/z=475.2 [M+H]⁺. ¹H NMR (300 MHz, DMSO-d₆) δ 10.50 (s, 1H), 9.12-9.01 (m, 2H), 8.95-8.85 (m, 1H), 8.64 (s, 1H), 8.43 (s, 1H), 8.08-7.92 (m, 2H), 5.41-5.34 (m, 1H), 4.41 (s, 3H), 4.34-4.23 (m, 2H), 4.01-3.94 (m, 2H), 3.40 (s, 3H), 3.26 (s, 3H), 2.47-2.41 (m, 2H), 2.16-2.05 (m, 2H).

### Example 2: 6-([4-[3-cyano-4-(oxan-4-yloxy)phenyl]pyrimidin-2-yl]amino)-N-(2-hydroxyethyl)-2-methoxypyridine-3-carboxamide (2)

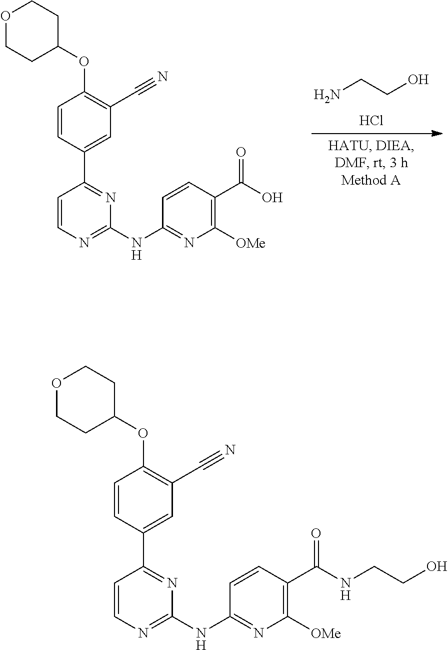

The title compound was prepared from 2-aminoethan-1-ol and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 30% to 60% gradient in 8 min; detector, UV 254 nm. 6-([4-[3-cyano-4-(oxan-4-yloxy)phenyl]pyrimidin-2-yl]amino)-N-(2-hydroxyethyl)-2-methoxypyridine-3-carboxamide was obtained as white solid (24 mg, 42%). HPLC: 98.9% purity, RT=1.60 min. MS: m/z=491.2 [M+H]⁺. ¹H NMR (300 MHz, DMSO-d₆) δ 10.10 (s, 1H), 8.72-8.58 (m, 2H), 8.58-8.46 (m, 1H), 8.34-8.24 (m, 1H), 8.19-8.08 (m, 1H), 8.05-7.96 (m, 1H), 7.71-7.62 (m, 1H), 7.61-7.52 (m, 1H), 5.01-4.89 (m, 1H), 4.87-4.77 (m, 1H), 4.03 (s, 3H), 3.95-3.81 (m, 2H), 3.63-3.47 (m, 4H), 3.44-3.34 (m, 2H), 2.11-2.00 (m, 2H), 1.79-1.61 (m, 2H).

### Example 3: 6-([4-[3-cyano-4-(oxan-4-yloxy)phenyl]pyrimidin-2-yl]amino)-N-(2-hydroxyethyl)-2-methoxy-N-methylpyridine-3-carboxamide (3)

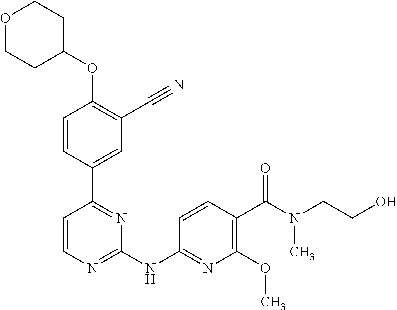 [SMILES: COc1nc(Nc2nccc(-c3ccc(OC4CCOCC4)c(C#N)c3)n2)ccc1C(=O)N(C)CCO]

The title compound was synthesized from 2-(methylamino)ethan-1-ol and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 35% to 65% gradient in 8 min; detector, UV 254 nm. 6-([4-[3-cyano-4-(oxan-4-yloxy)phenyl]pyrimidin-2-yl]amino)-N-(2-hydroxyethyl)-2-methoxy-N-methylpyridine-3-carboxamide was obtained as white solid (25 mg, 32%). HPLC: 99.6% purity, RT=2.68 min. MS: m/z=505.2 [M+H]⁺. ¹H NMR (300 MHz, DMSO-d₆) δ 9.93-9.83 (m, 1H), 8.69-8.58 (m, 2H), 8.55-8.45 (m, 1H), 7.96-7.86 (m, 1H), 7.69-7.51 (m, 3H), 5.01-4.91 (m, 1H), 4.81-4.65 (m, 1H), 3.95-3.81 (m, 5H), 3.63-3.39 (m, 5H), 3.22-3.16 (m, 1H), 2.98 and 2.88 (s and s, 3H), 2.10-1.99 (m, 2H), 1.78-1.60 (m, 2H).

### Example 4: 6-{4-[3-Cyano-4-(tetrahydro-pyran-4-yloxy)-phenyl]-pyrimidin-2-ylamino}-N-((S)-23-dihydroxy-propyl)-2-methoxy-nicotinamide (4)

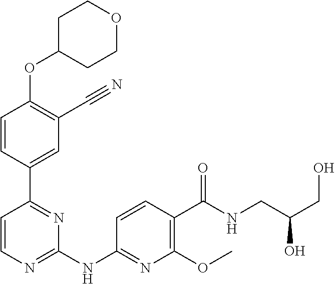 [SMILES: COc1nc(Nc2nccc(-c3ccc(OC4CCOCC4)c(C#N)c3)n2)ccc1C(=O)NC[C@H](O)CO]

The title compound (21 mg) was synthesized using 6-{4-[3-Cyano-4-(tetrahydro-pyran-4-yloxy)-phenyl]-pyrimidin-2-ylamino}-2-methoxy-nicotinic acid (30 mg), DIPEA (26 mg), HUTA (44 mg) and (S)-3-Amino-propane-1,2-diol (12 mg) using method A in 60% yield. ¹H NMR (DMSO-d6): 8.63 (m, 1H), 8.51 (1H), 7.94 (1H), 7.76 (m, 2H), 7.59 (m, 1H), 4.96 (s, 1H), 4.09 (1H), 3.90 (m, 5H), 3.57 (m, 1H), 3.47 (1H), 3.20 (1H), 2.90 (2H), 2.05 (m, 2H), 1.67 (2H), 1.47 (2H), 0.96 (2H). m/z: 521 [M+H]⁺.

### Example 5: 5-{2-[5-(1,1-Dioxo-thiomorpholine-4-carbonyl)-6-methoxy-pyridin-2-ylamino]-pyrimidin-4-yl}-2-(tetrahydro-pyran-4-yloxy)-benzonitrile (5)

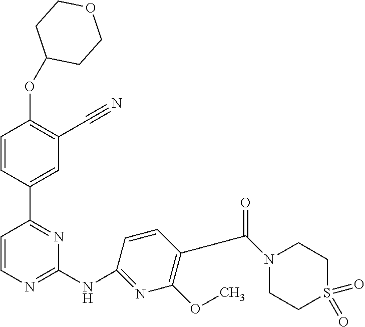 [SMILES: COc1nc(Nc2nccc(-c3ccc(OC4CCOCC4)c(C#N)c3)n2)ccc1CC(=O)N1CCS(=O)(=O)CC1]

The title compound (20 mg) was synthesized using 6-{4-[3-Cyano-4-(tetrahydro-pyran-4-yloxy)-phenyl]-pyrimidin-2-ylamino}-2-methoxy-nicotinic acid (50 mg), DIPEA (43 mg), HATU (74 mg) and 1,1-Dioxo-thiomorpholine (30 mg) in 31% yield using Method A. ¹H NMR (DMSO-d6): 8.63 (m, 1H), 8.51 (1H), 7.94 (1H), 7.76 (m, 2H), 7.59 (m, 1H), 4.96 (s, 1H), 4.09 (1H), 3.90 (m, 5H), 3.57 (m, 1H), 3.47 (1H), 3.20 (1H), 2.90 (2H), 2.05 (m, 2H), 1.74 (2H). m/z: 565 [M+H]⁺.

### Example 6: 5-{2-[6-methoxy-5-(3-oxo-piperazine-1-carbonyl)-pyridin-2-ylamino]-pyrimidin-4-yl}-2-(tetrahydro-pyran-4-yloxy)-benzonitrile (6)

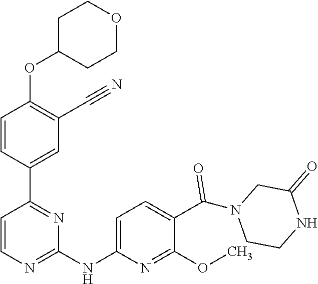 [SMILES: COc1nc(Nc2nccc(-c3ccc(OC4CCOCC4)c(C#N)c3)n2)ccc1C(=O)N1CCNC(=O)C1]

The title compound (22 mg) was synthesized using 6-{4-[3-Cyano-4-(tetrahydro-pyran-4-yloxy)-phenyl]-pyrimidin-2-ylamino}-2-methoxy-nicotinic acid (50 mg), DIPEA (43 mg), HUTA (74 mg) and piperazin-2-one (22 mg) in 37% yield using method A. ¹H NMR (DMSO-d6): 8.63 (m, 1H), 8.51 (1H), 7.94 (1H), 7.76 (m, 2H), 7.59 (m, 1H), 4.96 (s, 1H), 4.09 (1H), 3.90 (m, 5H), 3.57 (m, 1H), 3.47 (1H), 3.20 (1H), 2.90 (2H), 2.05 (m, 4H), 1.74 (4H). m/z: 530 [M+H]⁺.

### Example 7: 5-(2-[[6-methoxy-5-([6-oxa-3-azabicyclo[3.1.1]heptan-3-yl]carbonyl)pyridin-2-yl]amino]pyrimidin-4-yl)-2-(oxan-4-yloxy)benzonitrile (7)

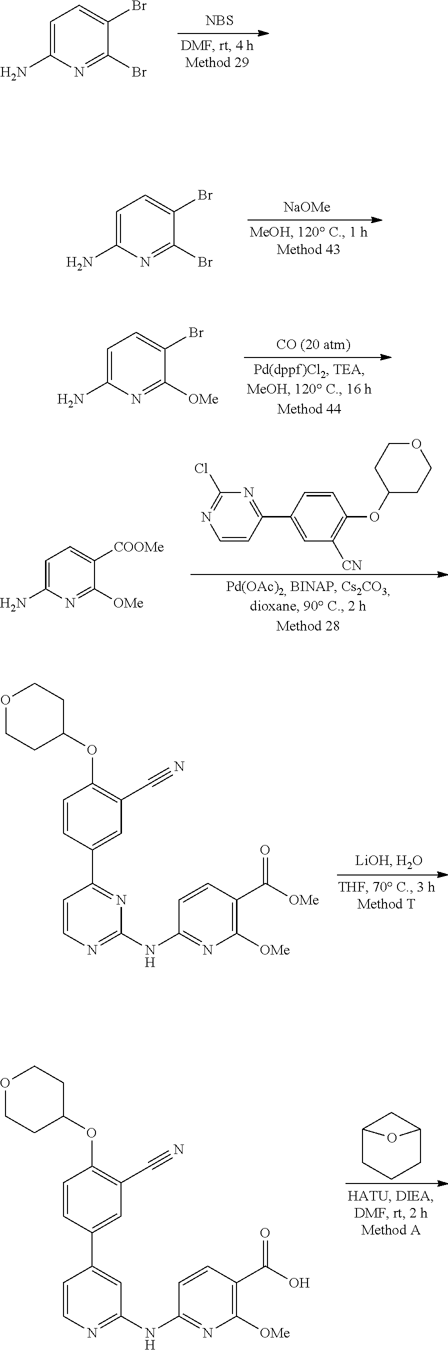

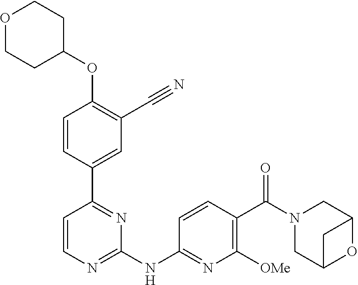 [SMILES: COc1nc(Nc2nccc(-c3ccc(OC4CCOCC4)c(C#N)c3)n2)ccc1C(=O)N1CC2CC(C1)O2]

5,6-dibromopyridin-2-amine: 5,6-dibromopyridin-2-amine was prepared from 6-bromopyridin-2-amine and NBS using Method 29. The final product was concentrated under reduced pressure to yield 6-amino-4-methoxy-N,N-dimethylpyridine-3-carboxamide as an yellow solid (10.00 g, 72%). MS: m/z=250.8 [M+H]⁺.

### Method 43

5-bromo-6-methoxypyridin-2-amine: To a solution of 5,6-dibromopyridin-2-amine (9.50 g, 37.71 mmol) in methanol (100 mL) was added NaOMe solution (30% in MeOH, 100 g, 555.55 mmol) at room temperature. The resulting mixture was stirred for 1 h at 120° C. When the reaction was done, it was quenched by the addition of phosphate buffer solution (200 mL, pH=7). The solids precipitated out from the resulting mixture were collected by filtration and dried under reduced pressure to yield 5-bromo-6-methoxypyridin-2-amine as orange solid (5.40 g, 71%). MS: m/z=202.8 [M+H]⁺.

### Method 44

methyl 6-amino-2-methoxypyridine-3-carboxylate: To a solution of 5-bromo-6-methoxypyridin-2-amine (4.50 g, 22.16 mmol) in methanol (50 mL) was added Pd(dppf)Cl₂.CH₂Cl₂ (950 mg, 1.16 mmol) under nitrogen atmosphere. The reaction tank was vacuumed and flushed with CO. Then the reaction mixture was stirred for 16 h at 120° C. under 20 atm CO atmosphere. When the reaction was done, the reaction mixture was concentrated under reduced pressure and the residue was purified by flash chromatography eluting with EtOAc in hexane (0% to 100% gradient) to yield methyl 6-amino-2-methoxypyridine-3-carboxylate as an yellow solid (2.07 g, 51%). MS: m/z=182.9 [M+H]⁺.

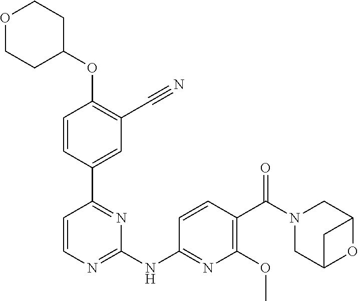 [SMILES: COc1nc(Nc2nccc(-c3ccc(OC4CCOCC4)c(C#N)c3)n2)ccc1C(=O)N1CC2CC(C1)O2]

The title compound was prepared from methyl 6-amino-2-methoxynicotinate, 5-(2-chloropyrimidin-4-yl)-2-(tetrahydro-2H-pyran-4-yloxy)benzonitrile, and 6-oxa-3-aza-bicyclo[3.1.1]heptane using Methods 28, T and A. The final product was purified by prep-HPLC under the following conditions: column, XBridge Shield RP18 OBD Column, 150×19 mm, 5 um; mobile phase, acetonitrile in water (with 0.05% NH₃.H₂O), 30% to 55% gradient in 8 min; detector, UV 254 nm. 5-(2-[[6-methoxy-5-([6-oxa-3-azabicyclo[3.1.1]heptan-3-yl]carbonyl)pyridin-2-yl]amino]pyrimidin-4-yl)-2-(oxan-4-yloxy)benzonitrile was obtained as a white solid (38 mg, 28% for 3 steps). HPLC: 98.8% purity, RT=1.50 min. MS: m/z=529.2 [M+H]⁺. ¹H NMR (400 MHz, Chloroform-d) δ 8.56 (d, J=5.2 Hz, 1H), 8.35-8.23 (m, 2H), 8.14-8.02 (m, 1H), 7.86 (s, 1H), 7.73-7.66 (m, 1H), 7.18 (d, J=5.3 Hz, 1H), 7.11 (d, J=9.0 Hz, 1H), 4.82-4.70 (m, 2H), 4.56-4.50 (m, 1H), 4.20-4.11 (m, 1H), 4.12-3.99 (m, 2H), 3.95 (s, 3H), 3.85-3.77 (m, 2H), 3.72-3.62 (m, 2H), 3.52-3.44 (m, 1H), 3.30-3.19 (m, 1H), 2.16-2.04 (m, 2H), 2.00-1.87 (m, 3H).

### Example 8: 6-([4-[3-cyano-4-(oxan-4-yloxy)phenyl]pyrimidin-2-yl]amino)-2-methoxy-N-[3-oxabicyclo[3.1.0]hexan-6-yl]pyridine-3-carboxamide (8)

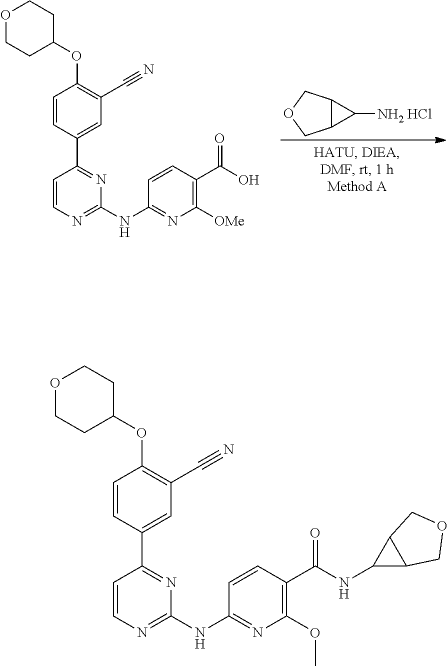

The title compound was prepared from 3-oxabicyclo[3.1.0]hexan-6-amine hydrochloride and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 35% to 65% gradient in 8 min; detector, UV 254 nm. 6-([4-[3-cyano-4-(oxan-4-yloxy)phenyl]pyrimidin-2-yl]amino)-2-methoxy-N-[3-oxabicyclo[3.1.0]hexan-6-yl]pyridine-3-carboxamide was obtained as a white solid (20 mg, 54%). HPLC: 95.8% purity, RT=1.96 min. MS: m/z=529.2 [M+H]⁺. ¹H NMR (300 MHz, Chloroform-d) δ 8.61-8.51 (m, 2H), 8.45-8.26 (m, 3H), 8.15-8.06 (m, 1H), 7.83-7.75 (m, 1H), 7.28-7.11 (m, 2H), 4.85-4.73 (m, 1H), 4.14-3.98 (m, 7H), 3.83-3.60 (m, 4H), 2.79-2.72 (m, 1H), 2.19-1.82 (m, 6H).

### Example 9: 6-{4-[3-Cyano-4-(tetrahydro-pyran-4-yloxy)-phenyl]-pyrimidin-2-ylamino}-N-(2-hydroxy-cyclopentyl)-2-methoxy-nicotinamide (9)

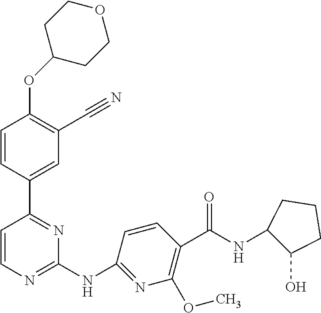 [SMILES: COc1nc(Nc2nccc(-c3ccc(OC4CCOCC4)c(C#N)c3)n2)ccc1C(=O)NC1CCC[C@@H]1O]

The title compound (24 mg) was synthesized from 6-{4-[3-Cyano-4-(tetrahydro-pyran-4-yloxy)-phenyl]-pyrimidin-2-ylamino}-2-methoxy-nicotinic acid (30 mg), DIPEA (26 mg), HUTA (44 mg) and 2-Amino-cyclopentanol (17 mg) using Method A in 67% yield. ¹H NMR (DMSO-d6): 8.63 (m, 1H), 8.51 (1H), 7.94 (1H), 7.76 (m, 2H), 7.59 (m, 1H), 4.96 (s, 1H), 4.09 (1H), 3.90 (m, 5H), 3.57 (m, 1H), 3.47 (1H), 3.20 (1H), 2.90 (2H), 2.05 (m, 2H), 1.67 (2H), 1.47 (2H), 0.96 (2H). m/z: 531 [M+H]⁺.

### Example 10: 5-(2-[[6-methoxy-5-([8-oxa-3-azabicyclo[3.2.1]octan-3-yl]carbonyl)pyridin-2-yl]amino]pyrimidin-4-yl)-2-(oxan-4-yloxy)benzonitrile (10)

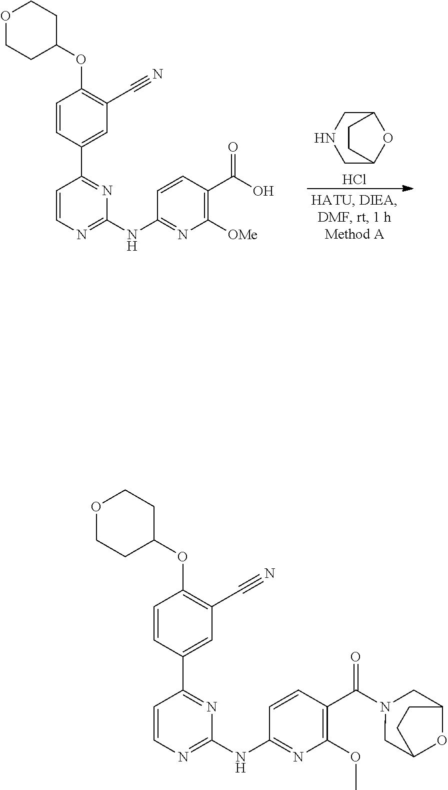 [SMILES: COc1nc(Nc2nccc(-c3ccc(OC4CCOCC4)c(C#N)c3)n2)ccc1C(=O)O ; C1CC2CNCC1O2 ; Cl ; COc1nc(Nc2nccc(-c3ccc(OC4CCOCC4)c(C#N)c3)n2)ccc1C(=O)N1CC2CCC(C1)O2]

The title compound was prepared from 8-oxa-3-azabicyclo[3.2.1]octane hydrochloride and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Shield RP18 OBD Column, 150×19 mm, 5 um; mobile phase, acetonitrile in water (with 0.05% NH₃.H₂O), 42% to 62% gradient in 8 min; detector, UV 254 nm. 5-(2-[[6-methoxy-5-([8-oxa-3-azabicyclo[3.2.1]octan-3-yl]carbonyl)pyridin-2-yl]amino]pyrimidin-4-yl)-2-(oxan-4-yloxy)benzonitrile was obtained as a white solid (25 mg, 66%). HPLC: 99.5% purity, RT=1.86 min. MS: m/z=543.2 [M+H]⁺. ¹H NMR (300 MHz, Chloroform-d) δ 8.59-8.51 (m, 1H), 8.41-8.22 (m, 2H), 8.09-7.98 (m, 2H), 7.67 (s, 1H), 7.23-7.07 (m, 2H), 4.83-4.71 (m, 1H), 4.49-4.33 (m, 2H), 4.28-4.21 (m, 1H), 4.12-3.97 (m, 2H), 3.95 (s, 3H), 3.74-3.60 (m, 2H), 3.47-3.41 (m, 1H), 3.20-3.08 (m, 2H), 2.22-1.65 (m, 8H).

### Example 11:5-(2-[[6-methoxy-5-([2-oxa-5-azabicyclo[2.2.2]octan-5-yl]carbonyl)pyridin-2-yl]amino]pyrimidin-4-yl)-2-(oxan-4-yloxy)benzonitrile (11)

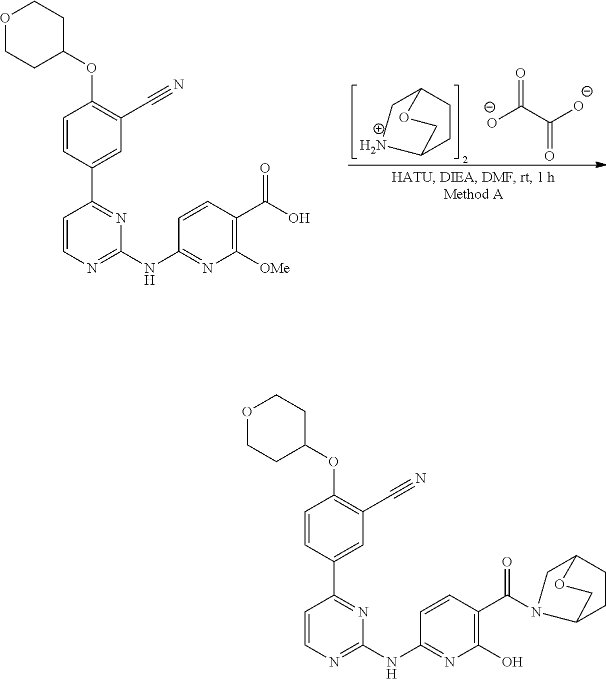

The title compound was prepared from bis(2-oxa-5-azabicyclo[2.2.2]octane) oxalic acid and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 40% to 70% gradient in 8 min; detector, UV 254 nm. 5-(2-[[6-methoxy-5-([2-oxa-5-azabicyclo[2.2.2]octan-5-yl]carbonyl)pyridin-2-yl]amino]pyrimidin-4-yl)-2-(oxan-4-yloxy)benzonitrile was obtained as a white solid (20 mg, 66%). HPLC: 99.8% purity, RT=1.19 min. MS: m/z=543.3 [M+H]⁺. ¹H NMR (300 MHz, Chloroform-d) δ 8.60-8.51 (m, 1H), 8.37-8.22 (m, 2H), 8.08-7.98 (m, 1H), 7.97-7.91 (m, 1H), 7.70 (d, J=8.1 Hz, 1H), 7.23-7.07 (m, 2H), 4.83-4.72 (m, 1H), 4.69-4.63 (m, 1H), 4.30-3.60 (m, 11H), 3.59-3.52 (m, 1H), 2.35-1.40 (m, 8H).

### Example 12: 5-(2-[[5-([hexahydro-1H-furo[3,4-c]pyrol-5-yl]carbonyl)-6-methoxypyridin-2-yl]amino]pyrimidin-4-yl)-2-(oxan-4-yloxy)benzonitrile (12)

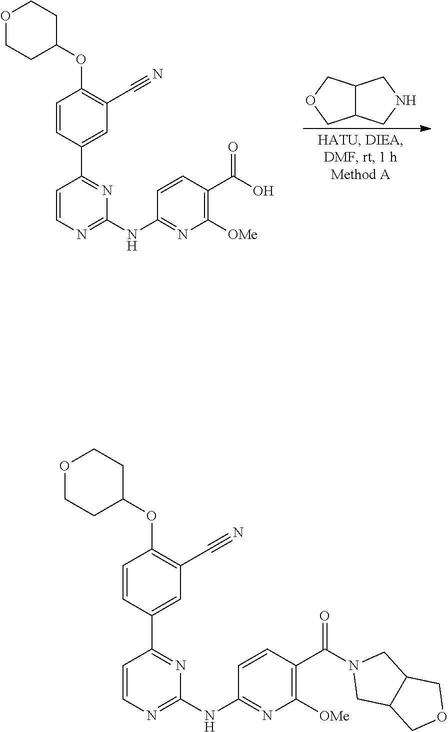 [SMILES: COc1nc(Nc2nccc(-c3ccc(OC4CCOCC4)c(C#N)c3)n2)ccc1C(=O)O ; C1NCC2COCC12 ; COc1nc(Nc2nccc(-c3ccc(OC4CCOCC4)c(C#N)c3)n2)ccc1C(=O)N1CC2COCC2C1]

The title compound was prepared from hexahydro-1H-furo[3,4-c]pyrrole and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Shield RP18 OBD Column, 150×19 mm, 5 um; mobile phase, acetonitrile in water (with 0.05% NH₃.H₂O), 30% to 55% gradient in 8 min; detector, UV 254 nm. 5-(2-[[5-([hexahydro-1H-furo[3,4-c]pyrrol-5-yl]carbonyl)-6-methoxypyridin-2-yl]amino]pyrimidin-4-yl)-2-(oxan-4-yloxy)benzonitrile was obtained as a white solid (40 mg, 88%). HPLC: 99.5% purity, RT=1.53 min. MS: m/z=543.2 [M+H]⁺. ¹H NMR (400 MHz, Chloroform-d) δ 8.56 (d, J=5.3 Hz, 1H), 8.34-8.23 (m, 2H), 8.06-7.99 (m, 1H), 7.86 (s, 1H), 7.74-7.67 (m, 1H), 7.21-7.08 (m, 2H), 4.82-4.72 (m, 1H), 4.09-3.83 (m, 8H), 3.75-3.52 (m, 6H), 3.30-3.21 (m, 1H), 3.10-2.83 (m, 2H), 2.16-2.04 (m, 2H), 2.00-1.87 (m, 2H).

### Example 13: 6-{4-[3-Cyano-4-(tetrahydro-pyran-4-yloxy)-phenyl]-pyrimidin-2-ylamino}-2-methoxy-pyridine-3-carbonyl)-piperidine-4-carboxylic acid (13)

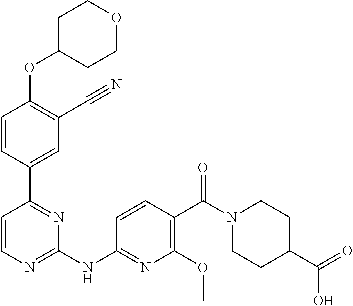 [SMILES: COc1nc(Nc2nccc(-c3ccc(OC4CCOCC4)c(C#N)c3)n2)ccc1C(=O)N1CCC(C(=O)O)CC1]

The title compound (20 mg) was synthesized from 6-{4-[3-Cyano-4-(tetrahydro-pyran-4-yloxy)-phenyl]-pyrimidin-2-ylamino}-2-methoxy-nicotinic acid (50 mg), DIPEA (43 mg), HUTA (74 mg) and piperidine-4-carboxylic acid (17 mg) in 31% yield. ¹H NMR (DMSO-d6): 8.63 (m, 1H), 8.51 (1H), 7.94 (1H), 7.76 (m, 2H), 7.59 (m, 1H), 4.96 (s, 1H), 4.09 (1H), 3.90 (m, 5H), 3.57 (m, 1H), 3.47 (1H), 3.20 (1H), 2.90 (2H), 2.05 (m, 2H), 1.67 (2H), 1.47 (2H), 0.96 (2H). m/z: 559 [M+H]⁺.

### Example 14: 6-([4-[3-cyano-4-(oxan-4-yloxy)phenyl]pyrimidin-2-yl]amino)-2-methoxy-N-(1-methylpiperidin-4-yl)pyridine-3-carboxamide (14)

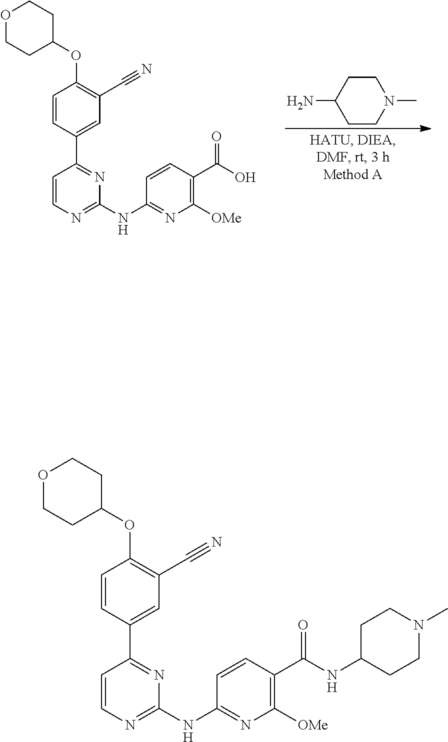

The title compound was prepared from 1-methylpiperidin-4-amine and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 46% to 60% gradient in 8 min; detector, UV 254 nm. 6-([4-[3-cyano-4-(oxan-4-yloxy)phenyl]pyrimidin-2-yl]amino)-2-methoxy-N-(1-methylpiperidin-4-yl)pyridine-3-carboxamide was obtained as a white solid (22 mg, 27%). HPLC: 97.2% purity, RT=1.41 min. MS: m/z=544.3 [M+H]⁺. ¹H NMR (300 MHz, DMSO-d₆) δ 10.09 (s, 1H), 8.72-8.58 (m, 2H), 8.56-8.46 (m, 1H), 8.26-8.17 (m, 1H), 8.04-7.95 (m, 1H), 7.88-7.79 (m, 1H), 7.71-7.62 (m, 1H), 7.62-7.52 (m, 1H), 5.02-4.90 (m, 1H), 4.03 (s, 3H), 3.95-3.81 (m, 2H), 3.81-3.74 (m, 1H), 3.63-3.49 (m, 2H), 2.71-2.61 (m, 2H), 2.17 (s, 3H), 2.11-2.01 (m, 4H), 1.90-1.44 (m, 6H).

### Example 15: 5-(2-{5-[4-(2-Hydroxy-ethyl)-piperazine-1-carbonyl]-6-methoxy-pyridin-2-ylamino}-pyrimidin-4-yl)-2-(tetrahydro-pyran-4-yloxy)-benzonitrile (15)

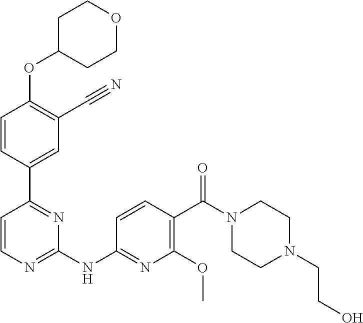 [SMILES: COc1nc(Nc2nccc(-c3ccc(OC4CCOCC4)c(C#N)c3)n2)ccc1C(=O)N1CCN(CCO)CC1]

The title compound (20 mg) was synthesized from 6-{4-[3-Cyano-4-(tetrahydro-pyran-4-yloxy)-phenyl]-pyrimidin-2-ylamino}-2-methoxy-nicotinic acid (30 mg), DIPEA (26 mg), HUTA (44 mg) and 2-Piperazin-1-yl-ethanol (17 mg) with Method A in 52% yield. ¹H NMR (DMSO-d6): 8.63 (m, 1H), 8.51 (1H), 7.94 (1H), 7.76 (m, 2H), 7.59 (m, 1H), 4.96 (s, 1H), 4.09 (1H), 3.90 (m, 5H), 3.57 (m, 1H), 3.47 (1H), 3.20 (1H), 2.90 (2H), 2.05 (m, 2H), 1.67 (2H), 1.47 (2H), 0.96 (2H). m/z: 560 [M+H]⁺.

### Example 16: 5-(2-[[6-methoxy-5-([3-oxa-9-azabicyclo[3.3.1]nonan-9-yl]carbonyl)pyridin-2-yl]amino]pyrimidin-4-yl)-2-(oxan-4-yloxy)benzonitrile (16)

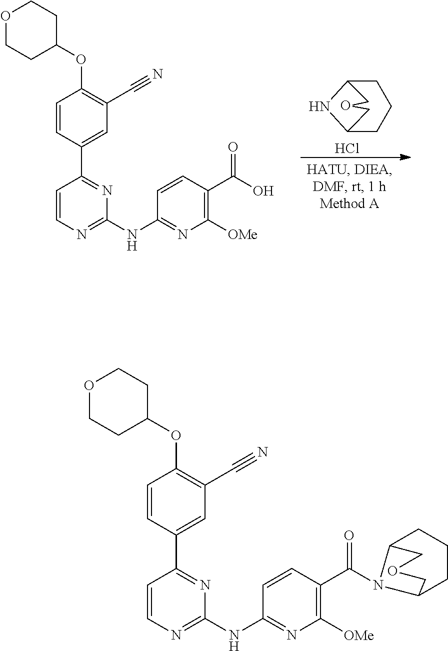 [SMILES: COc1nc(Nc2nccc(-c3ccc(OC4CCOCC4)c(C#N)c3)n2)ccc1C(=O)O ; C1CC2COCC(C1)N2 ; Cl ; COc1nc(Nc2nccc(-c3ccc(OC4CCOCC4)c(C#N)c3)n2)ccc1C(=O)N1C2CCCC1COC2]

The title compound was prepared from 3-oxa-9-azabicyclo[3.3.1]nonane hydrochloride and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 40% to 70% gradient in 8 min; detector, UV 254 nm. 5-(2-[[6-methoxy-5-([3-oxa-9-azabicyclo[3.3.1]nonan-9-yl]carbonyl)pyridin-2-yl]amino]pyrimidin-4-yl)-2-(oxan-4-yloxy)benzonitrile was obtained as a white solid (27 mg, 87%). HPLC: 99.6% purity, RT=3.13 min. MS: m/z=557.3 [M+H]⁺. ¹H NMR (300 MHz, Chloroform-d) δ 8.55 (d, J=5.3 Hz, 1H), 8.37-8.23 (m, 2H), 8.14-8.07 (m, 1H), 8.06-7.97 (m, 1H), 7.70 (d, J=8.0 Hz, 1H), 7.24-7.07 (m, 2H), 4.83-4.72 (m, 1H), 4.69-4.63 (m, 1H), 4.12-3.80 (m, 9H), 3.74-3.60 (m, 2H), 3.49-3.43 (m, 1H), 2.59-2.53 (m, 1H), 2.20-1.53 (m, 9H).

### Example 17: 5-(2-[[5-([7-hydroxy-3-oxa-9-azabicyclo[3.3.1]nonan-9-yl]carbonyl)-6-methoxypyridin-2-yl]amino]pyrimidin-4-yl)-2-(oxan-4-yloxy)benzonitrile (17)

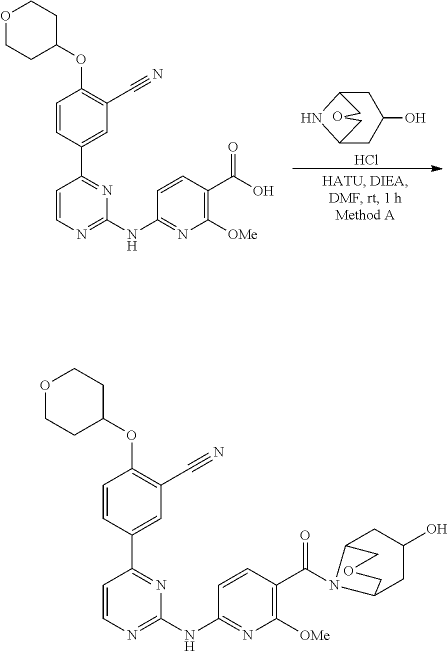 [SMILES: COc1nc(Nc2nccc(-c3ccc(OC4CCOCC4)c(C#N)c3)n2)ccc1C(=O)O ; OC1CC2COCC(C1)N2 ; Cl ; COc1nc(Nc2nccc(-c3ccc(OC4CCOCC4)c(C#N)c3)n2)ccc1C(=O)N1C2COCC1CC(O)C2]

The title compound was prepared from 3-oxa-9-azabicyclo[3.3.1]nonan-7-ol hydrochloride and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 35% to 65% gradient in 8 min; detector, UV 254 nm. 5-(2-[[5-([7-hydroxy-3-oxa-9-azabicyclo[3.3.1]nonan-9-yl]carbonyl)-6-methoxypyridin-2-yl]amino]pyrimidin-4-yl)-2-(oxan-4-yloxy)benzonitrile was obtained as a white solid (17 mg, 42%). HPLC: 99.3% purity, RT=1.29 min. MS: m/z=573.3 [M+H]⁺. ¹H NMR (300 MHz, Chloroform-d) δ 8.56 (d, J=5.3 Hz, 1H), 8.40-8.21 (m, 2H), 8.11-8.00 (m, 2H), 7.75-7.66 (m, 1H), 7.25-7.16 (m, 1H), 7.17-7.07 (m, 1H), 5.55-5.44 (m, 1H), 4.82-4.74 (m, 2H), 4.13-3.78 (m, 10H), 3.74-3.60 (m, 3H), 2.40-2.26 (m, 1H), 2.24-1.75 (m, 7H).

### Example 18: 5-(2-[[6-methoxy-5-([5-methyl-2,5-diazabicyclo[2.2.2]octan-2-yl]carbonyl)pyridin-2-yl]amino]pyrimidin-4-yl)-2-(oxan-4-yloxy)benzonitrile (18)

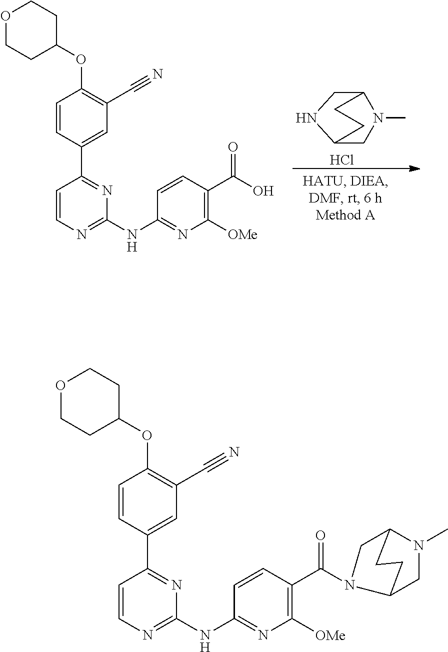

The title compound was prepared from 2-methyl-2,5-diazabicyclo[2.2.2]octane and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 40% to 70% gradient in 8 min; detector, UV 254 nm. 5-(2-[[6-methoxy-5-([5-methyl-2,5-diazabicyclo[2.2.2]octan-2-yl]carbonyl)pyridin-2-yl]amino]pyrimidin-4-yl)-2-(oxan-4-yloxy)benzonitrile was obtained as a white solid (30 mg, 43%). HPLC: 99.0% purity, RT=1.38 min. MS: m/z=556.3 [M+H]⁺. ¹H NMR (300 MHz, DMSO-de) δ 9.90 (s, 1H), 8.69-8.57 (m, 2H), 8.55-8.44 (m, 1H), 7.97-7.86 (m, 1H), 7.72-7.58 (m, 2H), 7.60-7.51 (m, 1H), 5.02-4.90 (m, 1H), 3.97-3.81 (m, 5H), 3.79-3.68 (m, 1H), 3.63-3.49 (m, 2H), 3.43-3.33 (m, 2H), 2.95-2.59 (m, 3H), 2.36-2.26 (m, 3H), 2.14-1.44 (m, 8H).

### Example 19 & Example 20: 6-([4-[3-cyano-4-(oxan-4-yloxy)phenyl]pyrimidin-2-yl]amino)-2-methoxy-N-[(1R,5S,6S)-3-methyl-3-azabicyclo[3.1.1]heptan-6-yl]pyridine-3-carboxamide & 6-([4-[3-cyano-4-(oxan-4-yloxy)phenyl]pyrimidin-2-yl]amino)-2-methoxy-N-[(1R,5S,6R)-3-methyl-3-azabicyclo[3.1.1]heptan-6-yl]pyridine-3-carboxamide

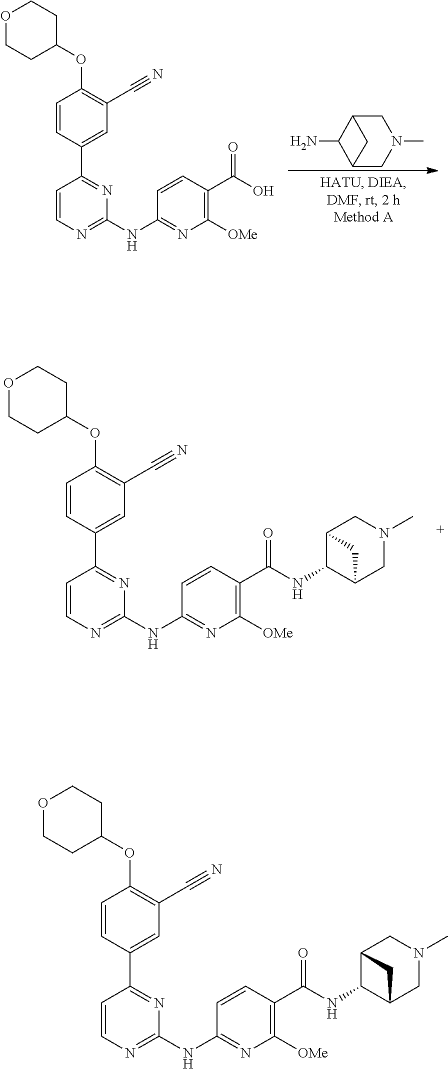

The title compounds were prepared from 3-methyl-3-aza-bicyclo[3.1.1]heptan-6-amine and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Prep C18 OBD Column, 150×19 mm, 5 um; mobile phase, acetonitrile in water (with 0.05% NH₃.H₂O), 30% to 60% gradient in 8 min; detector, UV 254 nm. The cis and trans isomers 6-([4-[3-cyano-4-(oxan-4-yloxy)phenyl]pyrimidin-2-yl]amino)-2-methoxy-N-[(1R,5S,6s)-3-methyl-3-azabicyclo[3.1.1]heptan-6-yl]pyridine-3-carboxamide and 6-([4-[3-cyano-4-(oxan-4-yloxy)phenyl]pyrimidin-2-yl]amino)-2-methoxy-N-[(1R,5S,6r)-3-methyl-3-azabicyclo[3.1.1]heptan-6-yl]pyridine-3-carboxamide were separated.

6-([4-[3-cyano-4-(oxan-4-yloxy)phenyl]pyrimidin-2-yl]amino)-2-methoxy-N-[(1R,5S,6S)-3-methyl-3-azabicyclo[3.1.1]heptan-6-yl]pyridine-3-carboxamide (20): (12 mg, 24%, light yellow solid) HPLC: 91.7% purity, RT=1.91 min. MS: m/z=556.3 [M+H]⁺. ¹H NMR (300 MHz, DMSO-d₆) δ 10.15 (s, 1H), 9.32 (d, J=9.5 Hz, 1H), 8.72-8.59 (m, 2H), 8.57-8.47 (m, 1H), 8.38-8.28 (m, 1H), 8.08-7.98 (m, 1H), 7.68 (d, J=5.3 Hz, 1H), 7.58 (d, J=9.2 Hz, 1H), 5.02-4.90 (m, 1H), 4.57-4.47 (m, 1H), 4.06 (s, 3H), 3.95-3.81 (m, 2H), 3.63-3.49 (m, 2H), 3.16-3.06 (m, 2H), 2.76-2.65 (m, 2H), 2.58-2.50 (m, 2H), 2.42 (s, 3H), 2.07-2.00 (m, 2H), 1.82-1.62 (m, 3H), 1.17-1.00 (m, 1H).

6-([4-[3-cyano-4-(oxan-4-yloxy)phenyl]pyrimidin-2-yl]amino)-2-methoxy-N-[(1R,5S,6R)-3-methyl-3-azabicyclo[3.1.1]heptan-6-yl]pyridine-3-carboxamide (19): (14 mg, 15%, off-white solid) HPLC:98.8% purity, RT=1.06 min. MS: m/z=556.4 [M+H]⁺. ¹H NMR (300 MHz, DMSO-d₆) δ 10.11 (s, 1H), 8.72-8.59 (m, 2H), 8.57-8.47 (m, 1H), 8.33-8.25 (m, 1H), 8.23-8.13 (m, 1H), 8.05-7.95 (m, 1H), 7.71-7.63 (m, 1H), 7.62-7.53 (m, 1H), 5.01-4.91 (m, 1H), 4.05 (s, 3H), 3.93-3.83 (m, 2H), 3.72-3.62 (m, 1H), 3.63-3.50 (m, 2H), 3.04-2.94 (m, 2H), 2.80-2.73 (m, 2H), 2.37-2.30 (m, 6H), 2.11-2.00 (m, 2H), 1.81-1.60 (m, 3H).

### Example 21: 6-((4-(3-cyano-4-((tetrahydro-2H-pyran-4-yl)oxy)phenyl)pyrimidin-2-yl)amino)-2-methoxy-N-(quinuclidin-3-yl)nicotinamide (21)

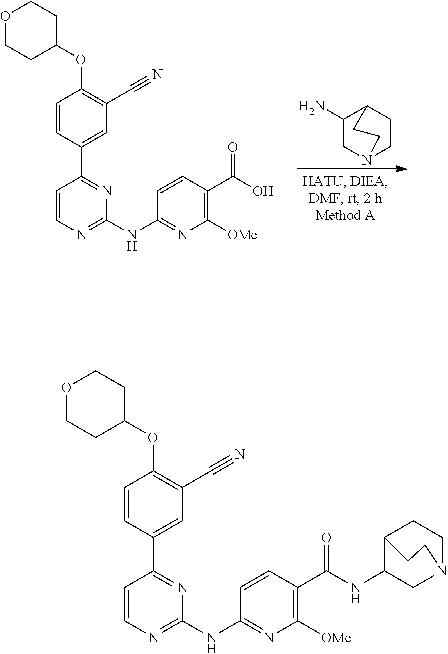 [SMILES: COc1nc(Nc2nccc(-c3ccc(OC4CCOCC4)c(C#N)c3)n2)ccc1C(=O)O ; NC1CN2CCC1CC2 ; COc1nc(Nc2nccc(-c3ccc(OC4CCOCC4)c(C#N)c3)n2)ccc1C(=O)NC1CN2CCC1CC2]

The title compound was prepared from 1-azabicyclo[2.2.2]octan-3-amine and 6-([4-[3-cyano-4-(oxan-4-yloxy)phenyl]pyrimidin-2-yl]amino)-2-methoxypyridine-3-carboxylic acid using Method A. The final product was purified by prep-HPLC under the following conditions: column, XBridge Shield RP18 OBD Column, 150×19 mm, 5 um; mobile phase, acetonitrile in water (with 0.05% NH₃.H₂O), 65% to 85% gradient in 8 min; detector, UV 254 nm. 6-((4-(3-cyano-4-((tetrahydro-2H-pyran-4-yl)oxy)phenyl)pyrimidin-2-yl)amino)-2-methoxy-N-(quinuclidin-3-yl)nicotinamide was obtained as off-white solid (9 mg, 8%). HPLC: 99.3% purity, RT=2.76 min. MS: m/z=556.3 [M+H]⁺. ¹H NMR (300 MHz, Methanol-d₄) δ 8.57 (d, J=5.3 Hz, 1H), 8.49-8.37 (m, 2H), 8.33-8.22 (m, 1H), 8.17-8.08 (m, 1H), 7.48-7.34 (m, 2H), 4.14-4.07 (m, 4H), 4.05-3.91 (m, 2H), 3.72-3.57 (m, 2H), 3.42-3.31 (m, 1H), 2.93-2.79 (m, 4H), 2.71-2.58 (m, 1H), 2.22-1.48 (m, 10H).

### Example 22: 6-([4-[3-cyano-4-(oxan-4-yloxy)phenyl]pyrimidin-2-yl]amino)-N,N-dimethylpyridine-3-carboxamide (22)

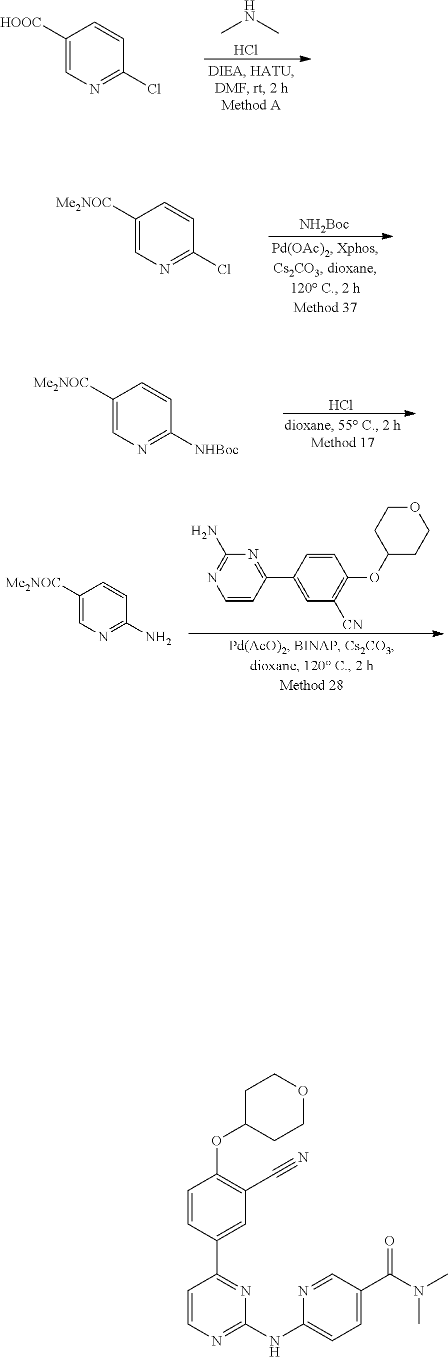

The title compound was prepared from 6-chloronicotinic acid, dimethylamine hydrochloride, tert-butyl carbamate, and 5-(2-aminopyrimidin-4-yl)-2-(tetrahydro-2H-pyran-4-yloxy)benzonitrile using Methods A, 37, 17, and 28. The final product was purified by prep-HPLC under the following conditions: column, XBridge Prep Phenyl OBD Column, 150×19 mm, 5 um; mobile phase, acetonitrile in water (with 10 mmol/L NH₄HCO₃), 20% to 40% gradient in 8 min; detector, UV 254 nm. 6-([4-[3-cyano-4-(oxan-4-yloxy)phenyl]pyrimidin-2-yl]amino)-N,N-dimethylpyridine-3-carboxamide was obtained as off-white solid (40 mg, 3.6% for 4 steps). HPLC: 98.1% purity, RT=1.31 min. MS: m/z=445.1 [M+H]⁺. ¹H NMR (300 MHz, Methanol-d₄) δ 8.75 (d, J=5.9 Hz, 1H), 8.65-8.58 (m, 1H), 8.57-8.46 (m, 2H), 8.29 (dd, J=9.0, 2.2 Hz, 1H), 7.86 (d, J=5.9 Hz, 1H), 7.49 (dd, J=22.8, 9.0 Hz, 2H), 5.00-4.90 (m, 1H), 4.05-3.91 (m, 2H), 3.72-3.58 (m, 2H), 3.11 (s, 6H), 2.17-2.04 (m, 2H), 1.92-1.74 (m, 2H).

### Example 23: 5-([4-[3-cyano-4-(oxan-4-yloxy)phenyl]pyrimidin-2-yl]amino)-N,N-dimethylpyridine-2-carboxamide (23)

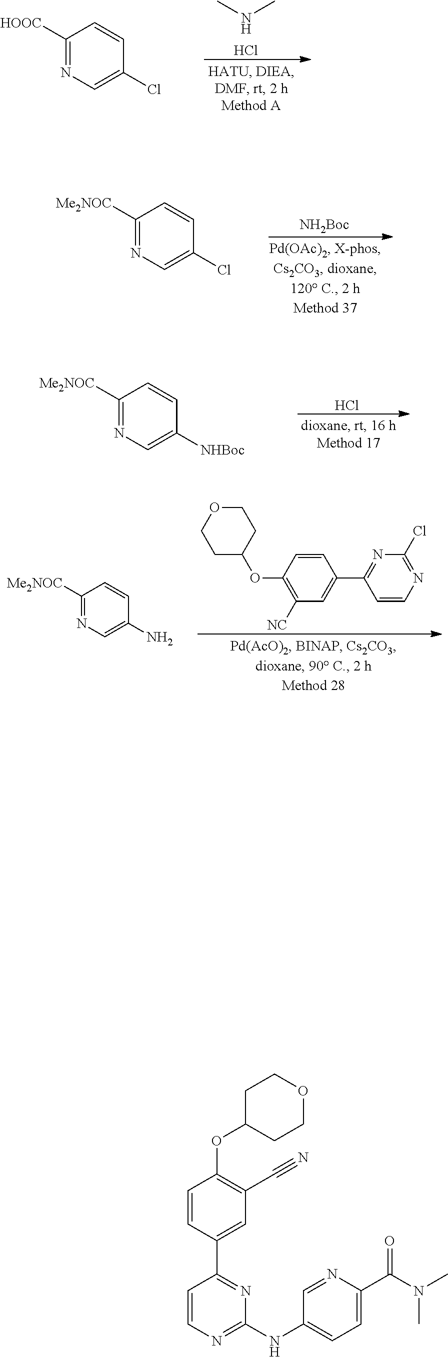

The title compound was prepared from 5-chloropicolinic acid, dimethylamine hydrochloride, tert-butyl carbamate, and 5-(2-chloropyrimidin-4-yl)-2-(tetrahydro-2H-pyran-4-yloxy)benzonitrile using Method A, 37, 17, and 28. The final product was purified by prep-HPLC under the following conditions: column, XBridge Prep Phenyl OBD Column, 150×19 mm, 5 um; mobile phase, acetonitrile in water (with 0.05% NH₃.H₂O), 35% to 65% gradient in 8 min; detector, UV 254 nm. 5-([4-[3-cyano-4-(oxan-4-yloxy)phenyl]pyrimidin-2-yl]amino)-N,N-dimethylpyridine-2-carboxamide was obtained as a white solid (65 mg, 13% for 4 steps). HPLC: 99.1% purity, RT=1.04 min. MS: m/z=445.1 [M+H]⁺. ¹H NMR (300 MHz, DMSO-d₆) δ 10.12 (s, 1H), 8.99-8.92 (m, 1H), 8.66-8.51 (m, 2H), 8.50-8.40 (m, 1H), 8.39-8.28 (m, 1H), 7.63-7.51 (m, 3H), 5.00-4.87 (m, 1H), 3.94-3.80 (m, 2H), 3.62-3.47 (m, 2H), 3.08-2.96 (m, 6H), 2.09-1.98 (m, 2H), 1.77-1.59 (m, 2H).

### Example 24: 6-([4-[6-cyano-5-(oxan-4-yloxy)pyridin-2-yl]pyrimidin-2-yl]amino)-4-methoxy-N,N-dimethylpyridine-3-carboxamide (24)

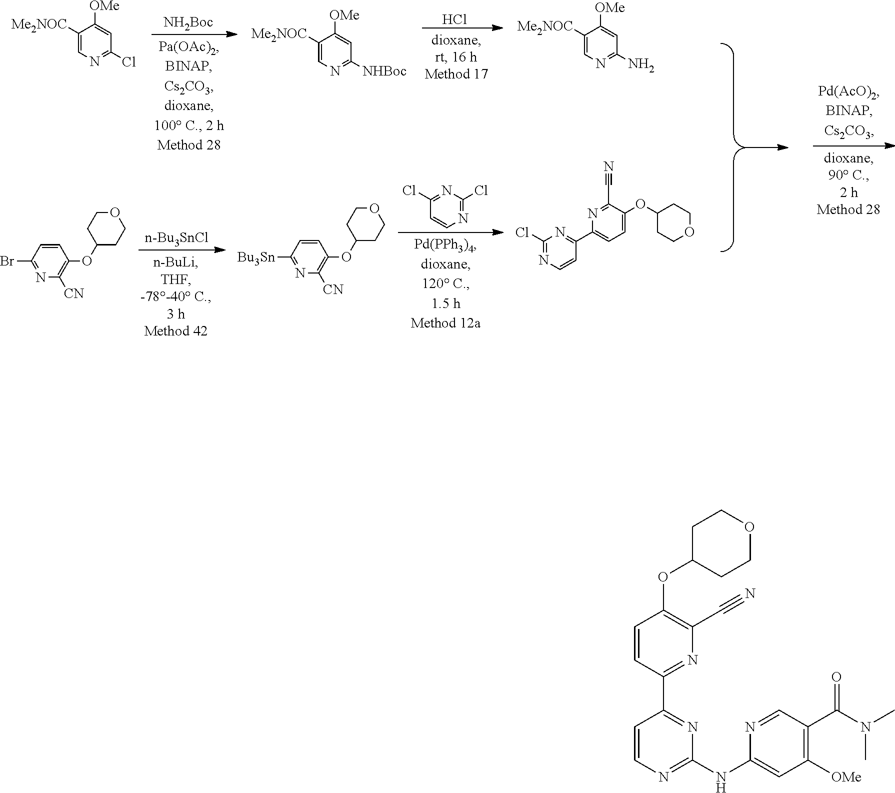

6-amino-4-methoxy-N,N-dimethylpyridine-3-carboxamide: 6-amino-4-methoxy-N,N-dimethylpyridine-3-carboxamide was prepared from 6-chloro-4-methoxy-N,N-dimethylnicotinamide and tert-butyl carbamate using Methods 17 and 28. The final product was concentrated under reduced pressure to yield 6-amino-4-methoxy-N,N-dimethylpyridine-3-carboxamide as an yellow solid (399 mg, 64% for 2 steps). MS: m/z=196.0 [M+H]⁺.

### Method 42

3-(oxan-4-yloxy)-6-(tributylstannyl)pyridine-2-carbonitrile: At −78° C., to a solution of 6-bromo-3-(oxan-4-yloxy)pyridine-2-carbonitrile (115 mg, 0.41 mmol) in THF (5 mL) was added n-BuLi in hexane (0.24 mL, 0.60 mmol, 2.5 M) dropwise. The resulting solution was stirred for 30 min at −78° C., and then was added by tributyl(chloro)stannane (158 mg, 0.48 mmol). The resulting mixture was stirred for 1 h at −78° C., warmed up to −40° C., and kept stirring for additional 2 h at −40° C. When the reaction was done, it was quenched by the addition of water (20 mL). The resulting mixture was extracted with ethyl acetate (30 mL×3). The organic phases were combined, washed with brine and dried over Na₂SO₄. The solvent was removed under reduced pressure and the residue was purified by flash chromatography eluting with EtOAc in hexane (0% to 15% gradient) to yield 3-(oxan-4-yloxy)-6-(tributylstannyl)pyridine-2-carbonitrile as an yellow oil (65 mg, 32%). MS: m/z=495.1 [M+H]⁺.

6-([4-[6-cyano-5-(oxan-4-yloxy)pyridin-2-yl]pyrimidin-2-yl]amino)-4-methoxy-N, N-dimethylpyridine-3-carboxamide: The title compound was prepared from 3-(tetrahydro-2H-pyran-4-yloxy)-6-(tributylstannyl)picolinonitrile, 2,4-dichloropyrimidine, and 6-amino-4-methoxy-N,N-dimethylnicotinamide using Method 28. The final product was purified by prep-HPLC under the following conditions: column, XBridge Prep Phenyl OBD Column, 150×19 mm, 5 um; mobile phase, acetonitrile in water (with 0.05% NH₃.H₂O), 25% to 50% gradient in 8 min; detector, UV 254 nm. 6-([4-[6-cyano-5-(oxan-4-yloxy)pyridin-2-yl]pyrimidin-2-yl]amino)-4-methoxy-N,N-dimethylpyridine-3-carboxamide was obtained as a white solid (10 mg, 19% for 2 steps). HPLC: 99.8% purity, RT=1.63 min. MS: m/z=476.1 [M+H]⁺. ¹H NMR (300 MHz, DMSO-d₆) δ 10.20 (s, 1H), 8.74 (d, J=5.1 Hz, 1H), 8.65 (d, J=9.1 Hz, 1H), 8.24-8.12 (m, 2H), 8.02 (s, 1H), 7.73 (d, J=5.1 Hz, 1H), 5.05-4.94 (m, 1H), 3.98 (s, 3H), 3.94-3.82 (m, 2H), 3.63-3.49 (m, 2H), 2.98 (s, 3H), 2.84 (s, 3H), 2.12-2.01 (m, 2H), 1.80-1.65 (m, 2H).

### Example 25: 5-([4-[6-cyano-5-(oxan-4-yloxy)pyridin-2-yl]pyrimidin-2-yl]amino)-3-methoxy-N,N-dimethylpyridine-2-carboxamide (25)

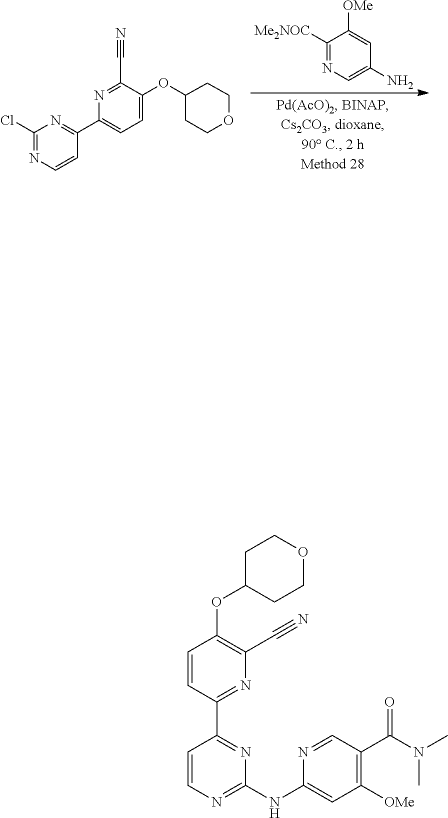

The title compound was prepared from 6-(2-chloropyrimidin-4-yl)-3-(tetrahydro-2H-pyran-4-yloxy)picolinonitrile and 5-amino-3-methoxy-N,N-dimethylpicolinamide using Method 28. The final product was purified by prep-HPLC under the following conditions: column, XBridge Prep Phenyl OBD Column, 150×19 mm, 5 um; mobile phase, acetonitrile in water (with 0.05% NH₃.H₂O), 15% to 45% gradient in 8 min; detector, UV 254 nm. 5-([4-[6-cyano-5-(oxan-4-yloxy)pyridin-2-yl]pyrimidin-2-yl]amino)-3-methoxy-N,N-dimethylpyridine-2-carboxamide was obtained as off-white solid (16 mg, 89%). HPLC: 97.3% purity, RT=3.32 min. MS: m/z=476.1 [M+H]⁺. 1H NMR (300 MHz, Methanol-d4) δ 8.72-8.62 (m, 2H), 8.59-8.51 (m, 1H), 8.35-8.28 (m, 1H), 7.93 (d, J=9.1 Hz, 1H), 7.79 (d, J=5.1 Hz, 1H), 5.01-4.92 (m, 1H), 4.09-3.94 (m, 5H), 3.75-3.61 (m, 2H), 3.14 (s, 3H), 2.93 (s, 3H), 2.16-2.09 (m, 2H), 1.96-1.81 (m, 2H).

### Example 26: 6-([4-[6-cyano-5-(oxan-4-yloxy)pyridin-2-yl]pyrimidin-2-yl]amino)-2-methoxy-N,N-dimethylpyridine-3-carboxamide (26)

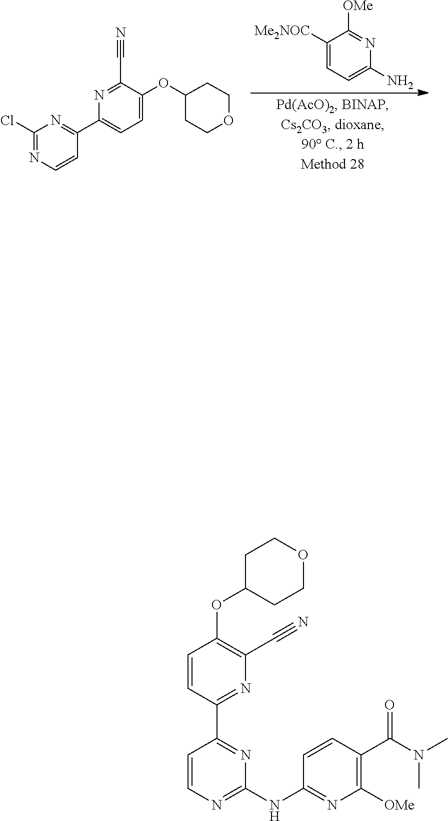

The title compound was prepared from 6-(2-chloropyrimidin-4-yl)-3-(tetrahydro-2H-pyran-4-yloxy)picolinonitrile and 6-amino-2-methoxy-N,N-dimethylnicotinamide using Method 28. The final product was purified by prep-HPLC under the following conditions: column, XBridge Prep Phenyl OBD Column, 150×19 mm, 5 um; mobile phase, acetonitrile in water (with 0.05% NH₃.H₂O), 30% to 55% gradient in 8 min; detector, UV 254 nm. 6-([4-[6-cyano-5-(oxan-4-yloxy)pyridin-2-yl]pyrimidin-2-yl]amino)-2-methoxy-N,N-dimethylpyridine-3-carboxamide was obtained as a white solid (27 mg, 37%). HPLC: 99.1% purity, RT=11.2 min. MS: m/z=476.2 [M+H]⁺. ¹H NMR (300 MHz, DMSO-d₆) δ 9.97 (s, 1H), 8.75-8.58 (m, 2H), 8.19-8.09 (m, 1H), 7.95-7.86 (m, 1H), 7.75-7.59 (m, 2H), 5.02-4.95 (m, 1H), 3.94-3.81 (m, 5H), 3.60-3.47 (m, 2H), 2.95 (s, 3H), 2.82 (s, 3H), 2.06-1.99 (m, 2H), 1.75-1.68 (m, 2H).

### Example 27: 6-([4-[6-cyano-5-(oxan-4-yloxy)pyridin-2-yl]pyrimidin-2-yl]amino)-2-methoxy-N,N-dimethylpyridine-3-carboxamide (27)

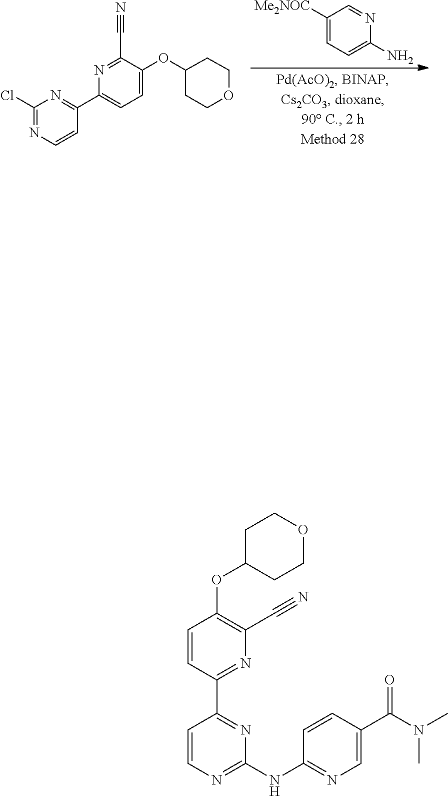

6-([4-[6-cyano-5-(oxan-4-yloxy)pyridin-2-yl]pyrimidin-2-yl]amino)-N,N-dimethylpyridine-3-carboxamide. 6-([4-[6-cyano-5-(oxan-4-yloxy)pyridin-2-yl]pyrimidin-2-yl]amino)-N,N-dimethylpyridine-3-carboxamide was prepared from 6-(2-chloropyrimidin-4-yl)-3-(tetrahydro-2H-pyran-4-yloxy)picolinonitrile and 6-amino-N,N-dimethylnicotinamide using Method 28. The final product was purified by prep-HPLC under the following conditions: column, XBridge Prep Phenyl OBD Column, 150×19 mm, 5 um; mobile phase, acetonitrile in water (with 0.05% NH₃.H₂O), 25% to 55% gradient in 8 min; detector, UV 254 nm. 6-([4-[6-cyano-5-(oxan-4-yloxy)pyridin-2-yl]pyrimidin-2-yl]amino)-N,N-dimethylpyridine-3-carboxamide was obtained as a white solid (29 mg, 30%). HPLC: 97.0% purity, RT=1.00 min. MS: m/z=446.2 [M+H]⁺. ¹H NMR (300 MHz, DMSO-d₆) δ 10.31 (s, 1H), 8.78-8.69 (m, 1H), 8.69-8.59 (m, 1H), 8.44-8.32 (m, 2H), 8.20-8.10 (m, 1H), 7.93-7.84 (m, 1H), 7.78-7.68 (m, 1H), 5.03-4.96 (m, 1H), 3.94-3.84 (m, 2H), 3.62-3.49 (m, 2H), 3.01 (s, 6H), 2.08-2.01 (m, 2H), 1.77-1.67 (m, 2H).

### Example 28: 5-([4-[6-cyano-5-(oxan-4-yloxy)pyridin-2-yl]pyrimidin-2-yl]amino)-N,N-dimethylpyridine-2-carboxamide (28)

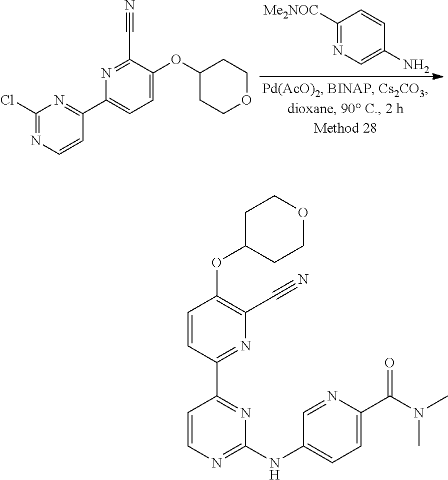

The title compound was prepared from 6-(2-chloropyrimidin-4-yl)-3-(tetrahydro-2H-pyran-4-yloxy)picolinonitrile and 5-amino-N,N-dimethylpicolinamide using Method 28. The final product was purified by prep-HPLC under the following conditions: column, XBridge Prep Phenyl OBD Column, 150×19 mm, 5 um; mobile phase, acetonitrile in water (with 0.05% NH₃.H₂O), 15% to 45% gradient in 8 min; detector, UV 254 nm. 5-([4-[6-cyano-5-(oxan-4-yloxy)pyridin-2-yl]pyrimidin-2-yl]amino)-N,N-dimethylpyridine-2-carboxamide was obtained as off-white solid (35 mg, 20%). HPLC: 99.5% purity, RT=1.58 min. MS: m/z=446.1 [M+H]⁺. ¹H NMR (300 MHz, Methanol-d₄) δ 9.03-8.95 (m, 1H), 8.72-8.60 (m, 2H), 8.51-8.41 (m, 1H), 7.92 (d, J=9.1 Hz, 1H), 7.79 (d, J=5.0 Hz, 1H), 7.63 (d, J=8.7 Hz, 1H), 5.02-4.91 (m, 1H), 4.08-3.95 (m, 2H), 3.75-3.61 (m, 2H), 3.18-3.09 (m, 6H), 2.19-2.08 (m, 2H), 1.96-1.78 (m, 2H).

### Example 29: N-[1-azabicyclo[2.2.2]octan-3-yl]-6-([4-[6-cyano-5-(oxan-4-yloxy)pyridin-2-yl]pyrimidin-2-yl]amino)-2-methoxypyridine-3-carboxamide (29)

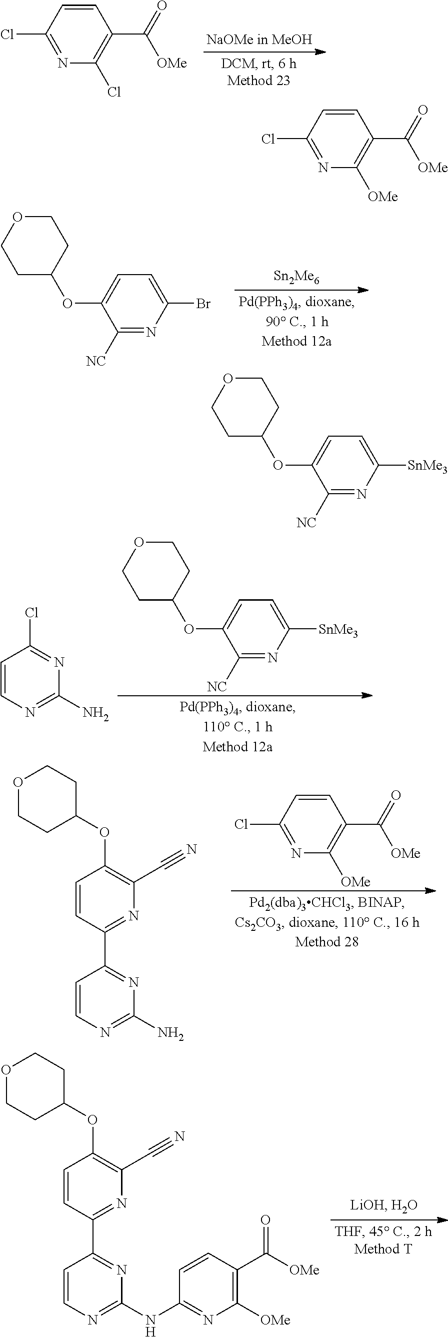

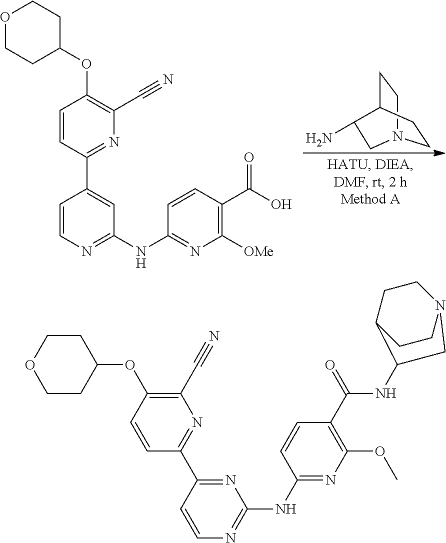 [SMILES: COc1nc(Nc2cc(-c3ccc(OC4CCOCC4)c(C#N)n3)ccn2)ccc1C(=O)O ; NC1CN2CCC1CC2 ; COc1nc(Nc2nccc(-c3ccc(OC4CCOCC4)c(C#N)n3)n2)ccc1C(=O)NC1CN2CCC1CC2]

The title compound was prepared from 4-chloropyrimidin-2-amine, 3-(tetrahydro-2H-pyran-4-yloxy)-6-(trimethylstannyl)picolinonitrile, methyl 6-chloro-2-methoxynicotinate and quinuclidin-3-amine using Methods 23, 12a, 28, T and A. The final product was purified by prep-HPLC under the following conditions: column, XBridge Prep OBD C18 Column, 150×19 mm, 5 um; mobile phase, acetonitrile in water (with 10 mmol/L NH₄HCO₃ and 0.1% NH₃.H₂O), 40% to 80% gradient in 8 min; detector, UV 254 nm. N-[1-azabicyclo[2.2.2]octan-3-yl]-6-([4-[6-cyano-5-(oxan-4-yloxy)pyridin-2-yl]pyrimidin-2-yl]amino)-2-methoxypyridine-3-carboxamide was obtained as a white solid (15 mg, 2.1% for 6 steps). HPLC: 92.5% purity, RT=1.42 min. MS: m/z=557.1 [M+H]⁺. ¹H NMR (300 MHz, DMSO-d₆) δ 10.23 (s, 1H), 8.82-8.60 (m, 2H), 8.27-7.95 (m, 4H), 7.79-7.71 (m, 1H), 5.06-4.93 (m, 1H), 4.13-3.80 (m, 6H), 3.63-3.49 (m, 2H), 3.28-3.14 (m, 1H), 2.96-2.52 (m, 5H), 2.14-1.32 (m, 9H).

### Example 30: 6-[2-([6-methoxy-5-[(piperidin-1-yl)carbonyl]pyridin-2-yl]amino)pyrimidin-4-yl]-3-(oxan-4-yloxy)pyridine-2-carbonitrile (30)

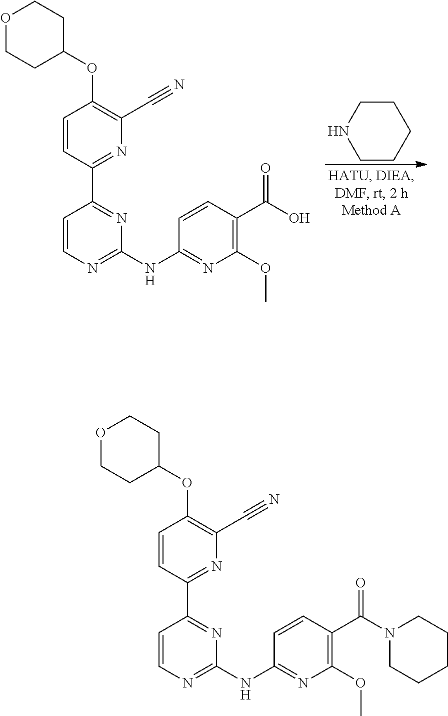 [SMILES: COc1nc(Nc2nccc(-c3ccc(OC4CCOCC4)c(C#N)n3)n2)ccc1C(=O)O ; C1CCNCC1 ; COc1nc(Nc2nccc(-c3ccc(OC4CCOCC4)c(C#N)n3)n2)ccc1C(=O)N1CCCCC1]

The title compound was prepared from piperidine and 6-([4-[6-cyano-5-(oxan-4-yloxy)pyridin-2-yl]pyrimidin-2-yl]amino)-2-methoxypyridine-3-carboxylic acid using Method A. The final product was purified by prep-HPLC under the following conditions: column, XBridge Shield RP18 OBD Column, 150×19 mm, 5 um; mobile phase, acetonitrile in water (with 0.05% NH₃.H₂O), 54% to 73% gradient in 8 min; detector, UV 254 nm. 6-[2-([6-methoxy-5-[(piperidin-1-yl)carbonyl]pyridin-2-yl]amino)pyrimidin-4-yl]-3-(oxan-4-yloxy)pyridine-2-carbonitrile was obtained as a white solid (19 mg, 39%). HPLC: 94.0% purity, RT=1.76 min. MS: m/z=516.3 [M+H]⁺. ¹H NMR (300 MHz, DMSO-d₆) δ 10.00 (s, 1H), 8.76-8.60 (m, 2H), 8.20-8.10 (m, 1H), 7.97-7.87 (m, 1H), 7.76-7.59 (m, 2H), 5.06-4.94 (m, 1H), 3.94-3.81 (m, 5H), 3.69-3.47 (m, 4H), 3.20-3.13 (m, 2H), 2.11-2.00 (m, 2H), 1.82-1.32 (m, 8H).

### Example 31: 6-[2-([6-methoxy-5-[(4-methylpiperazin-1-yl)carbonyl]pyridin-2-yl]amino)pyrimidin-4-yl]-3-(oxan-4-yloxy)pyridine-2-carbonitrile (31)

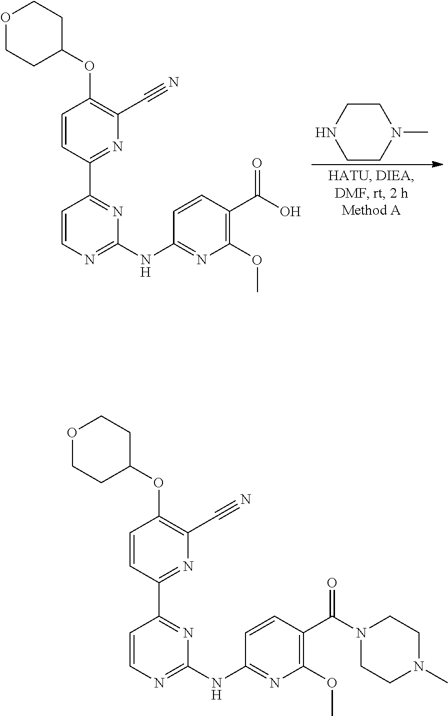

The title compound was prepared from 1-methylpiperazine and 6-([4-[6-cyano-5-(oxan-4-yloxy)pyridin-2-yl]pyrimidin-2-yl]amino)-2-methoxypyridine-3-carboxylic acid using Method A. The final product was purified by prep-HPLC under the following conditions: column, XBridge Shield RP18 OBD Column, 150×19 mm, 5 um; mobile phase, acetonitrile in water (with 0.05% NH₃.H₂O), 41% to 50% gradient in 8 min; detector, UV 254 nm. 6-[2-([6-methoxy-5-[(4-methylpiperazin-1-yl)carbonyl]pyridin-2-yl]amino)pyrimidin-4-yl]-3-(oxan-4-yloxy)pyridine-2-carbonitrile was obtained as a white solid (14 mg, 28%). HPLC: 93.8% purity, RT=5.29 min. MS: m/z=531.3 [M+H]⁺. ¹H NMR (300 MHz, DMSO-d₆) δ 10.03 (s, 1H), 8.78-8.61 (m, 2H), 8.21-8.11 (m, 1H), 7.99-7.89 (m, 1H), 7.77-7.62 (m, 2H), 5.09-4.95 (m, 1H), 4.03-3.79 (m, 5H), 3.74-3.47 (m, 4H), 3.38-3.07 (m, 4H), 2.41-1.95 (m, 7H), 1.81-1.61 (m, 2H).

### Example 32: 6-(2-[[6-methoxy-5-([6-oxa-3-azabicyclo[3.1.1]heptan-3-yl]carbonyl)pyridin-2-yl]amino]pyrimidin-4-yl)-3-(oxan-4-yloxy)pyridine-2-carbonitrile (32)

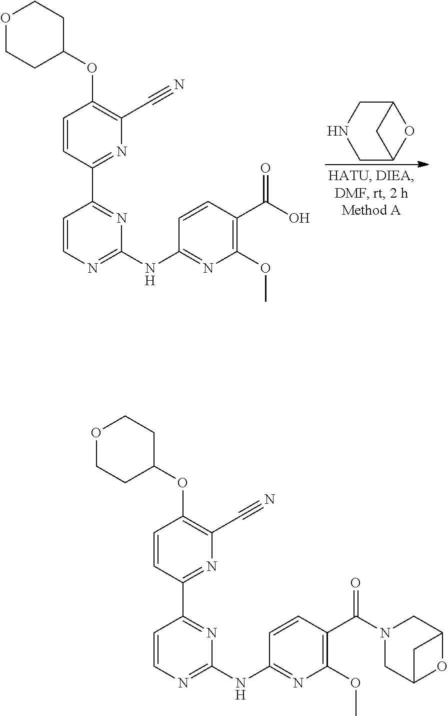 [SMILES: COc1nc(Nc2nccc(-c3ccc(OC4CCOCC4)c(C#N)n3)n2)ccc1C(=O)O ; C1NCC2CC1O2 ; COc1nc(Nc2nccc(-c3ccc(OC4CCOCC4)c(C#N)n3)n2)ccc1C(=O)N1CC2CC(C1)O2]

The title compound was prepared from 6-oxa-3-azabicyclo[3.1.1]heptane and 6-([4-[6-cyano-5-(oxan-4-yloxy)pyridin-2-yl]pyrimidin-2-yl]amino)-2-methoxypyridine-3-carboxylic acid using Method A. The final product was purified by prep-HPLC under the following conditions: column, XBridge Shield RP18 OBD Column, 150×19 mm, 5 um; mobile phase, acetonitrile in water (with 0.05% NH₃.H₂O), 40% to 56% gradient in 8 min; detector, UV 254 nm. 6-(2-[[6-methoxy-5-([6-oxa-3-azabicyclo[3.1.1]heptan-3-yl]carbonyl)pyridin-2-yl]amino]pyrimidin-4-yl)-3-(oxan-4-yloxy)pyridine-2-carbonitrile was obtained as a white solid (17 mg, 24%). HPLC: 99.1% purity, RT=2.44 min. MS: m/z=530.1 [M+H]⁺. ¹H NMR (300 MHz, DMSO-d₆) δ 10.03 (s, 1H), 8.78-8.61 (m, 2H), 8.20-8.11 (m, 1H), 8.00-7.90 (m, 1H), 7.78-7.68 (m, 2H), 5.07-4.95 (m, 1H), 4.69-4.62 (m, 1H), 4.53-4.46 (m, 1H), 3.98-3.82 (m, 6H), 3.68-3.50 (m, 5H), 3.14-3.04 (m, 1H), 2.12-2.02 (m, 2H), 1.86-1.65 (m, 3H).

### Example 33: 5-(2-(6-methoxy-5-(4-methylpiperazin-1-yl)pyridin-2-ylamino)pyrimidin-4-yl)-2-(tetrahydro-2H-pyran-4-yloxy)benzonitrile (33)

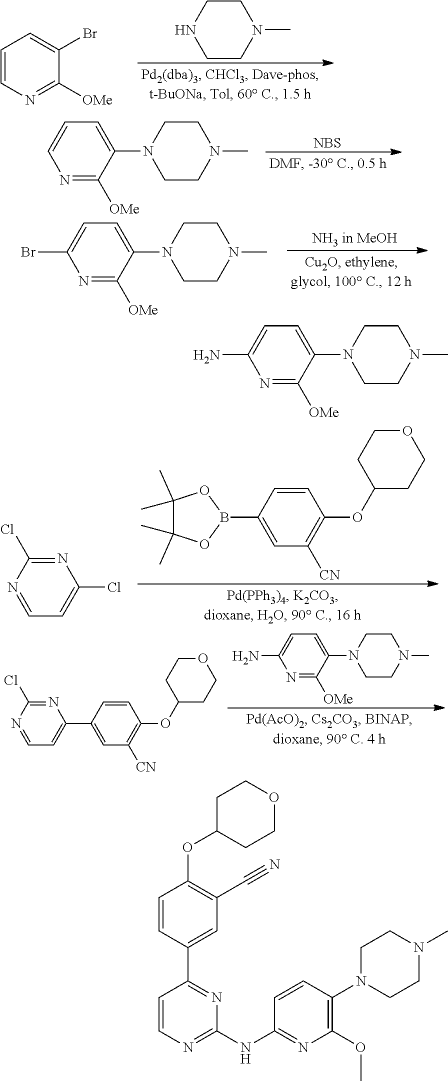

1-(2-methoxypyridin-3-yl)-4-methylpiperazine: To a solution of 3-bromo-2-methoxypyridine (950 mg, 5.05 mmol) in toluene (10 mL) was added 1-methylpiperazine (685 mg, 6.85 mmol), Pd₂(dba)₃CHCl₃ (265 mg, 0.26 mmol), Davephos (303 mg, 0.77 mmol), t-BuONa (739 mg, 7.69 mmol) at room temperature. The resulting solution was stirred for 1.5 h at 60° C. After cooling to room temperature, the reaction was then quenched by the addition of water (20 mL). The resulting solution was extracted with ethyl acetate (50 mL×3). The organic phases were combined, washed with brine and dried over Na₂SO₄. The solution was concentrated under reduced pressure and the residue was purified by flash chromatography eluting with MeOH in EtOAc (0% to 90% gradient) to yield 1-(2-methoxypyridin-3-yl)-4-methylpiperazine as brown oil (625 mg, 60%). MS: m/z=208.3 [M+H]⁺.

1-(6-bromo-2-methoxypyridin-3-yl)-4-methylpiperazine: At −30° C., to a solution of 1-(2-methoxypyridin-3-yl)-4-methylpiperazine (625 mg, 3.02 mmol) in DMF (14 mL) was slowly added a solution of NBS (637 mg, 3.58 mmol) in DMF (7 mL). The resulting solution was stirred for 30 min at −30° C. When the reaction was done, the reaction was then quenched by the addition of water (20 mL). The resulting solution was extracted with ethyl acetate (50 mL×3). The organic phases were combined, washed with brine and dried over Na₂SO₄. The solution was concentrated under reduced pressure and the residue was purified by flash chromatography eluting with MeOH in EtOAc (0% to 80% gradient) to yield 1-(6-bromo-2-methoxypyridin-3-yl)-4-methylpiperazine as a brown solid (745 mg, 86%). MS: m/z=286.2 [M+H]⁺.

6-methoxy-5-(4-methylpiperazin-1-yl)pyridin-2-amine: To a solution of 1-(6-bromo-2-methoxypyridin-3-yl)-4-methylpiperazine (745 mg, 2.60 mmol) in ethane-1,2-diol (9 mL) was added a solution of NH₃ (9 mL, 24 mmol, 7M), Cu₂O (24 mg, 0.17 mmol) at room temperature. The resulting solution was stirred for 12 h at 100° C. After cooling to room temperature, the reaction was then quenched by the addition of water (20 mL). The resulting solution was extracted with ethyl acetate (50 mL×3). The organic phases were combined, washed with brine and dried over Na₂SO₄. The solution was concentrated under reduced pressure and the residue was applied onto C18 gel column and purified by flash chromatography eluting with MeCN in water 0% to 1% gradient in 30 min to yield 6-methoxy-5-(4-methylpiperazin-1-yl)pyridin-2-amine as brown oil (265 mg, 46%). MS: m/z=223.2 [M+H]⁺.

5-(2-chloropyrimidin-4-yl)-2-(tetrahydro-2H-pyran-4-yloxy)benzonitrile: To a solution of 2,4-dichloropyrimidine (3 g, 20.14 mmol) in dioxane (30 mL) was added 2-(oxan-4-yloxy)-5-(tetramethyl-1,3,2-dioxaborolan-2-yl)benzonitrile (6.6 g, 20.05 mmol), Pd(PPh₃)₄ (400 mg, 0.35 mmol), potassium carbonate (5.4 g, 39.07 mmol), H₂O (9 mL) at room temperature. The resulting solution was stirred for 16 h at 90° C. After cooling to room temperature, the reaction was then quenched by the addition of water (150 mL). The resulting solution was extracted with ethyl acetate (250 mL×3). The organic phases were combined, washed with brine and dried over Na₂SO₄. The solution was concentrated under reduced pressure and the residue was purified by flash chromatography eluting with EtOAc in hexane (0% to 70% gradient) to yield 5-(2-chloropyrimidin-4-yl)-2-(oxan-4-yloxy)benzonitrile as a gray solid (2.80 g, 44%). MS: m/z=316.3 [M+H]⁺.

5-(2-(6-methoxy-5-(4-methylpiperazin-1-yl)pyridin-2-ylamino)pyrimidin-4-yl)-2-(tetrahydro-2H-pyran-4-yloxy)benzonitrile: To a solution of 5-(2-chloropyrimidin-4-yl)-2-(oxan-4-yloxy)benzonitrile (7 mg, 0.02 mmol) in dioxane (1 mL) was added 6-methoxy-5-(4-methylpiperazin-1-yl)pyridin-2-amine (10 mg, 0.04 mmol), Pd(OAc)₂ (1 mg, 0.20 equiv), BINAP (5.6 mg, 0.01 mmol), Cs₂CO₃ (22 mg, 0.06 mmol) at room temperature. The resulting solution was stirred for 4 h at 90° C. After cooling to room temperature, the reaction was then quenched by the addition of water (3 mL). The resulting solution was extracted with DCM (10 mL×3). The organic phases were combined, washed with brine and dried over Na₂SO₄. The solvent was removed under reduced pressure and the residue was purified by prep-HPLC under the following conditions: Column, XBridge Prep C18 OBD Column, 19×150 mm 5 um; MeCN in water (with 0.05% NH₃.H₂O), 20% to 40% gradient in 8 min; Detector, UV 254 nm. 5-(2-[[6-methoxy-5-(4-methylpiperazin-1-yl)pyridin-2-yl]amino]pyrimidin-4-yl)-2-(oxan-4-yloxy)benzonitrile as a yellow solid (9.7 mg, 90%). HPLC: 96.6% purity, RT=1.42 min. MS: m/z=502.2 [M+H]⁺. ¹H NMR (300 MHz, Methanol-d₄, ppm) δ 8.52-8.36 (m, 3H), 7.86 (d, j=8.3 Hz, 1H), 7.42-7.27 (m, 3H), 4.05-3.91 (m, 6H), 3.70-3.58 (m, 2H), 3.24-3.04 (m, 4H), 2.68-2.54 (m, 4H), 2.33 (s, 3H), 2.13-2.03 (m, 2H), 1.88-1.78 (m, 2H).

### Example 34: 5-[2-[(5-[[2-(dimethylamino)ethyl](methyl)amino]-6-methoxypyridin-2-yl)amino]pyrimidin-4-yl]-2-(oxan-4-yloxy)benzonitrile hydrochloride (34)

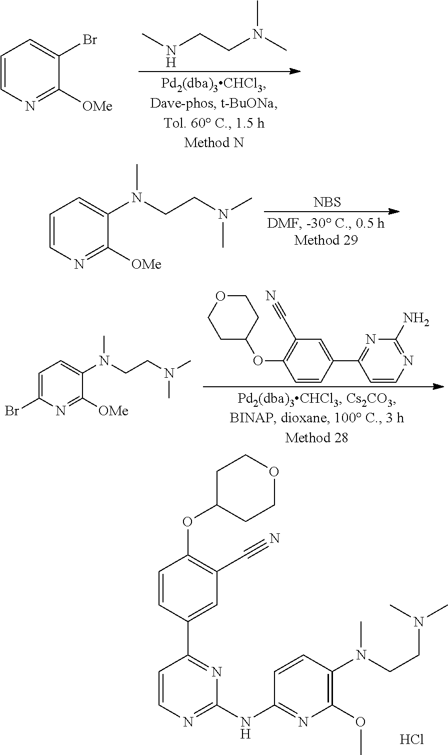

### Method N1

N-[2-(dimethylamino)ethyl]-2-methoxy-N-methylpyridin-3-amine: To a solution of 3-bromo-2-methoxypyridine (950 mg, 5.05 mmol) in toluene (10 mL) were added [2-(dimethylamino)ethyl](methyl)amine (618 mg, 6.04 mmol), Pd₂(dba)₃CHCl₃ (265 mg, 0.26 mmol), Davephos (303 mg, 0.77 mmol) and t-BuONa (739 mg, 7.69 mmol) at room temperature. The resulting mixture was stirred for 1.5 h at 60° C. When the reaction was done, the reaction mixture was concentrated under reduced pressure and the residue was purified by flash chromatography eluting with EtOAc in hexane (0% to 53% gradient) to yield N-[2-(dimethylamino)ethyl]-2-methoxy-N-methylpyridin-3-amine as an yellow solid (349 mg, 33%). MS: m/z=210.0 [M+H]⁺.

### Method 29

6-bromo-N-[2-(dimethylamino)ethyl]-2-methoxy-N-methylpyridin-3-amine: At −30° C., to a solution of N-[2-(dimethylamino)ethyl]-2-methoxy-N-methylpyridin-3-amine (179 mg, 0.86 mmol) in N,N-dimethylformamide (4 mL) was added a solution of NBS (166 mg, 0.93 mmol) in N,N-dimethylformamide (2 mL) slowly. The resulting mixture was stirred for 30 min at −30° C. When the reaction was done, the reaction mixture was diluted with H₂O (10 mL) and extracted with ethyl acetate (30 mL×3). The organic phases were combined, washed with brine and dried over Na₂SO₄. The solvent was removed under reduced pressure and the residue was purified by reverse phase flash chromatography eluting with MeOH in water (1% to 68% gradient in 30 min) to yield 6-bromo-N-[2-(dimethylamino)ethyl]-2-methoxy-N-methylpyridin-3-amine as brown solid (39 mg, 16%). MS: m/z=288.0 [M+H]⁺.

### Method 28a

5-[2-[(5-[[2-(dimethylamino)ethyl](methyl)amino]-6-methoxypyridin-2-yl)amino]pyrimidin-4-yl]-2-(oxan-4-yloxy)benzonitrile hydrochloride: To a solution of 6-bromo-N-[2-(dimethylamino)ethyl]-2-methoxy-N-methylpyridin-3-amine (68 mg, 0.24 mmol) in 1,4-dioxane (14 mL) were added 5-(2-aminopyrimidin-4-yl)-2-(oxan-4-yloxy)benzonitrile (69 mg, 0.23 mmol), Pd₂(dba)₃CHCl₃ (12 mg, 0.01 mmol), BINAP (15 mg, 0.02 mmol) and Cs₂CO₃ (150 mg, 0.46 mmol) at room temperature. The resulting mixture was stirred for 3 h at 100° C. When the reaction was done, the reaction mixture was concentrated under reduced pressure and the residue was purified by was purified by prep-HPLC under the following conditions: column, XBridge Prep C18 OBD Column, 150×19 mm, 5 um; mobile phase, acetonitrile in water (with 0.05% HCl), 30% to 50% gradient in 8 min; detector, UV 254 nm. 5-[2-[(5-[[2-(dimethylamino)ethyl](methyl)amino]-6-methoxypyridin-2-yl)amino]pyrimidin-4-yl]-2-(oxan-4-yloxy)benzonitrile hydrochloride was obtained as an yellow solid (21 mg, 17%). HPLC: 97.1% purity, RT=2.22 min. MS: m/z=504.2 [M+H]⁺. ¹H NMR (300 MHz, Methanol-d₄) δ 8.75-8.63 (m, 2H), 8.65-8.54 (m, 1H), 7.89-7.80 (m, 1H), 7.76-7.66 (m, 1H), 7.55-7.45 (m, 1H), 6.98-6.89 (m, 1H), 5.03-4.96 (m, 1H), 4.22 (s, 3H), 4.04-3.90 (m, 2H), 3.73-3.58 (m, 2H), 3.46-3.40 (m, 4H), 2.99 (s, 6H), 2.87 (s, 3H), 2.18-2.05 (m, 2H), 1.91-1.75 (m, 2H).

### Example 35: 2-[[6-([4-[3-cyano-4-(oxan-4-yloxy)phenyl]pyrimidin-2-yl]amino)-2-methoxypyridin-3-yl](methyl)amino]-N,N-dimethylacetamide hydrochloride (35)

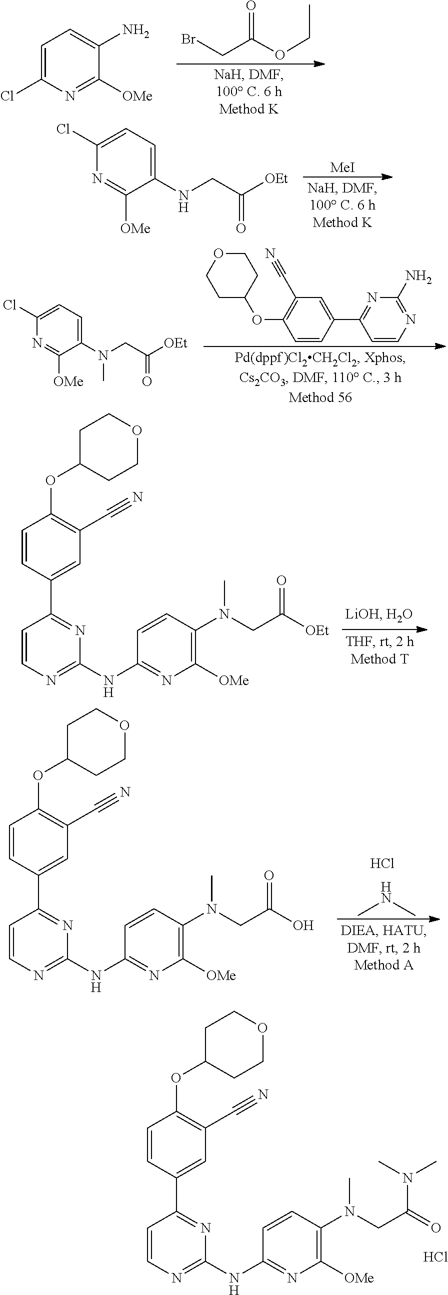

### Method K

Ethyl 2-[(6-chloro-2-methoxypyridin-3-yl)amino]acetate: To a solution of 6-chloro-2-methoxypyridin-3-amine (210 mg, 1.32 mmol) in N,N-dimethylformamide (5 mL) was added sodium hydride (35 mg, 1.45 mmol) at 0° C. The resulting mixture was stirred for 30 min at 0° C., and then was added by ethyl 2-bromoacetate (299 mg, 1.79 mmol) slowly. The reaction mixture was then stirred for 6 h at 100° C. When the reaction was done, it was quenched by the addition of water (10 mL) and the resulting mixture was extracted with ethyl acetate (20 mL×3). The organic phases were combined, washed with brine and dried over Na₂SO₄. The solvent was removed under reduced pressure to yield ethyl 2-[(6-chloro-2-methoxypyridin-3-yl)amino]acetate as a yellow solid (223 mg, 69%). MS: m/z=259.0 [M+H]⁺.

### Method 56

ethyl 2-[(6-chloro-2-methoxypyridin-3-yl)(methyl)amino]acetate: To a solution of 5-(2-aminopyrimidin-4-yl)-2-(oxan-4-yloxy)benzonitrile (107 mg, 0.36 mmol) in N,N-dimethylformamide (2 mL) was added ethyl 2-[(6-chloro-2-methoxypyridin-3-yl)(methyl)amino]acetate (71 mg, 0.27 mmol), Pd(dppf)Cl₂.CH₂Cl₂ (37 mg, 0.05 mmol), XPhos (36 mg, 0.08 mmol), CS₂CO₃ (247 mg, 0.76 mmol) at room temperature. The resulting mixture was stirred for 3 h at 110° C. When the reaction was done, the solids were filtered out. The solution was concentrated under reduced pressure and the residue was purified by flash chromatography eluting with EtOAc in hexane (0% to 100% gradient) to yield ethyl 2-[[6-([4-[3-cyano-4-(oxan-4-yloxy)phenyl]pyrimidin-2-yl]amino)-2-methoxypyridin-3-yl](methyl)amino]acetate as a yellow solid (122 mg, 86%). MS: m/z=519.8 [M+H]⁺.

2-[[6-([4-[3-cyano-4-(oxan-4-yloxy)phenyl]pyrimidin-2-yl]amino)-2-methoxypyridin-3-yl](methyl)amino]-N,N-dimethylacetamide hydrochloride: The title compound was prepared from ethyl 2-((6-(4-(3-cyano-4-(tetrahydro-2H-pyran-4-yloxy)phenyl)pyrimidin-2-ylamino)-2-methoxypyridin-3-yl)(methyl)amino)acetate and dimethylamine hydrochloride using Method T and A. The final product was purified by prep-HPLC under the following conditions: column, XBridge Prep C18 OBD Column, 150×19 mm, 5 um; mobile phase, acetonitrile in water (with 0.05% HCl), 30% to 60% gradient in 8 min; detector, UV 254 nm. 2-[[6-([4-[3-cyano-4-(oxan-4-yloxy)phenyl]pyrimidin-2-yl]amino)-2-methoxypyridin-3-yl](methyl)amino]-N,N-dimethylacetamide hydrochloride was obtained as brown solid (7 mg, 5.4% for 2 steps). HPLC: 97.2% purity, RT=1.04 min. MS: m/z=518.3 [M+H]⁺. ¹H NMR (300 MHz, Methanol-d₄) δ 8.69-8.38 (m, 3H), 7.84-7.44 (m, 4H), 4.98-4.86 (m, 1H), 4.44 (s, 2H), 3.97 (s, 3H), 3.90-3.76 (m, 2H), 3.56 (s, 1H), 3.68-3.51 (m, 2H), 3.00 (s, 3H), 2.92 (s, 3H), 2.77 (s, 3H), 2.07-1.94 (m, 2H), 1.75-1.56 (m, 2H).

### Example 36: 5-[2-([6-methoxy-5-[cis-3,4,5-trimethylpiperazin-1-yl]pyridin-2-yl]amino)pyrimidin-4-yl]-2-(oxan-4-yloxy)benzonitrile (36)

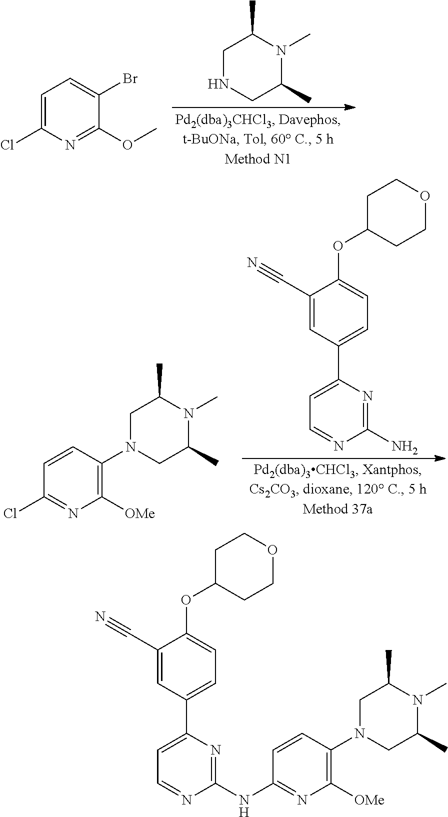

The title compound was prepared from 3-bromo-6-chloro-2-methoxypyridine, (2R,6S)-1,2,6-trimethylpiperazine and 5-(2-aminopyrimidin-4-yl)-2-(tetrahydro-2H-pyran-4-yloxy)benzonitrile using Method N1 and 37a. The final product was purified by prep-HPLC under the following conditions: column, XBridge Shield RP18 OBD Column, 150×19 mm, 5 um; mobile phase, acetonitrile in water (with 10 mmol/L NH₄HCO₃ and 0.1% NH₃.H₂O), 35% to 62% gradient in 8 min; detector, UV 254 nm. 5-[2-([6-methoxy-5-[cis-3,4,5-trimethylpiperazin-1-yl]pyridin-2-yl]amino)pyrimidin-4-yl]-2-(oxan-4-yloxy)benzonitrile was obtained as a light yellow solid (26 mg, 13% for 2 steps). HPLC: 95.5% purity, RT=4.32 min. MS: m/z=530.2 [M+H]⁺. ¹H NMR (300 MHz, Chloroform-d) δ 8.55-8.47 (m, 1H), 8.37-8.20 (m, 2H), 7.91-7.82 (m, 1H), 7.65 (s, 1H), 7.29-7.19 (m, 1H), 7.15-7.05 (m, 2H), 4.83-4.69 (m, 1H), 4.12-3.94 (m, 5H), 3.74-3.60 (m, 2H), 3.37-3.27 (m, 2H), 2.70-2.27 (m, 7H), 2.18-1.83 (m, 4H), 1.19 (br s, 6H).

### Example 37: 5-[2-([5-[cis-2,6-dimethylmorpholin-4-yl]-6-methoxypyridin-2-yl]amino)pyrimidin-4-yl]-2-(oxan-4-yloxy)benzonitrile (37)

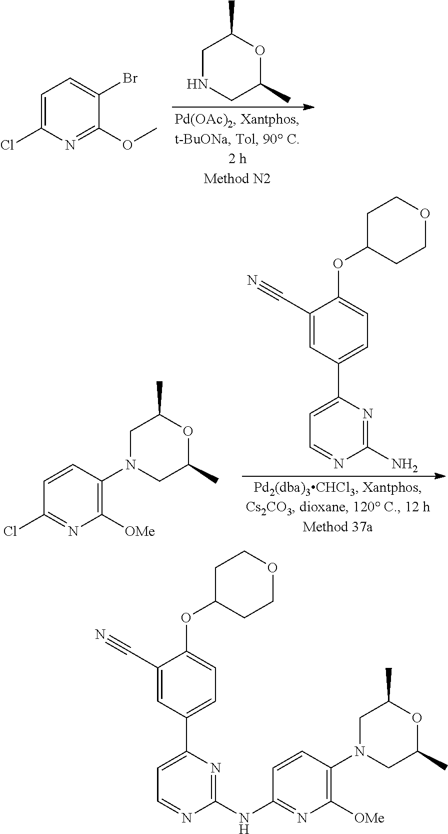

The title compound was prepared from 3-bromo-6-chloro-2-methoxypyridine, (2R,6S)-2,6-dimethylmorpholine and 5-(2-aminopyrimidin-4-yl)-2-(tetrahydro-2H-pyran-4-yloxy)benzonitrile using Method N2 and 37a. The final product was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 52% to 60% gradient in 8 min; detector, UV 254 nm. 5-[2-([5-[cis-2,6-dimethylmorpholin-4-yl]-6-methoxypyridin-2-yl]amino)pyrimidin-4-yl]-2-(oxan-4-yloxy)benzonitrile was obtained as an yellow solid (24 mg, 4.6% for 2 steps). HPLC: 95.0% purity, RT=5.89 min. MS: m/z=517.2 [M+H]⁺. ¹H NMR (300 MHz, Chloroform-d) δ 8.49 (d, J=5.2 Hz, 1H), 8.37-8.17 (m, 2H), 7.90-7.80 (m, 1H), 7.65 (s, 1H), 7.25-7.16 (m, 1H), 7.12-6.97 (m, 2H), 4.80-4.68 (m, 1H), 4.09-3.85 (m, 7H), 3.71-3.57 (m, 2H), 3.33-3.23 (m, 2H), 2.38-2.24 (m, 2H), 2.14-1.79 (m, 4H), 1.26-1.17 (m, 6H).

### Example 38: 5-[2-([5-[cis-3,5-dimethyl-4-(oxetan-3-yl)piperazin-1-yl]-6-methoxypyridin-2-yl]amino)pyrimidin-4-yl]-2-(oxan-4-yloxy)benzonitrile (38)

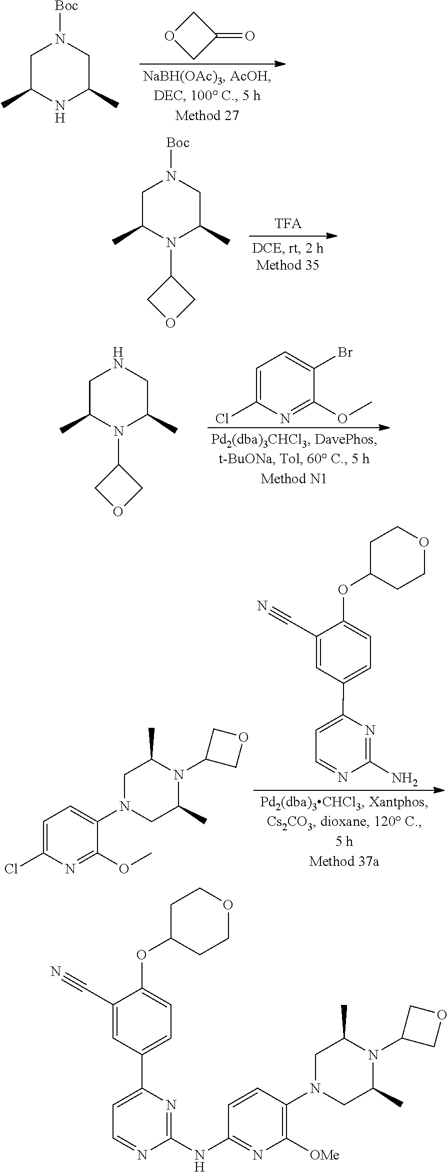

5-[2-([5-[cis-3,5-dimethyl-4-(oxetan-3-yl)piperazin-1-yl]-6-methoxypyridin-2-yl]amino)pyrimidin-4-yl]-2-(oxan-4-yloxy)benzonitrile: 5-[2-([5-[cis-3,5-dimethyl-4-(oxetan-3-yl)piperazin-1-yl]-6-methoxypyridin-2-yl]amino)pyrimidin-4-yl]-2-(oxan-4-yloxy)benzonitrile was prepared from (3S,5R)-tert-butyl 3,5-dimethylpiperazine-1-carboxylate, oxetan-3-one, 3-bromo-6-chloro-2-methoxypyridine and 5-(2-aminopyrimidin-4-yl)-2-(tetrahydro-2H-pyran-4-yloxy)benzonitrile using Method 27, 35, N1 and 37a. The final product was purified by prep-HPLC under the following conditions: column, XBridge Shield RP18 OBD Column, 150×19 mm, 5 um; mobile phase, acetonitrile in water (with 10 mmol/L NR₄HCO₃ and 0.1% NH₃.H₂O), 30% to 50% gradient in 8 min; detector, UV 254 nm. 5-[2-([5-[cis-3,5-dimethyl-4-(oxetan-3-yl)piperazin-1-yl]-6-methoxypyridin-2-yl]amino)pyrimidin-4-yl]-2-(oxan-4-yloxy)benzonitrile was obtained as a light yellow solid (20 mg, 0.2% for 4 steps). HPLC: 99.4% purity, RT=4.64 min. MS: m/z=572.1 [M+H]⁺. ¹H NMR (300 MHz, DMSO-d₆) δ 9.35 (s, 1H), 8.68-8.43 (m, 3H), 7.78-7.68 (m, 1H), 7.59-7.47 (m, 2H), 7.27-7.18 (m, 1H), 4.99-4.88 (m, 1H), 4.56-4.47 (m, 4H), 4.14-3.80 (m, 6H), 3.62-3.48 (m, 2H), 3.05-2.59 (m, 6H), 2.10-1.99 (m, 2H), 1.75-1.61 (m, 2H), 1.07-0.98 (m, 6H).

### Example 39: 5-(2-[[6-methoxy-5-(piperidin-4-yl)pyridin-2-yl]amino]pyrimidin-4-yl)-2-(oxan-4-yloxy)benzonitrile hydrochloride (39)

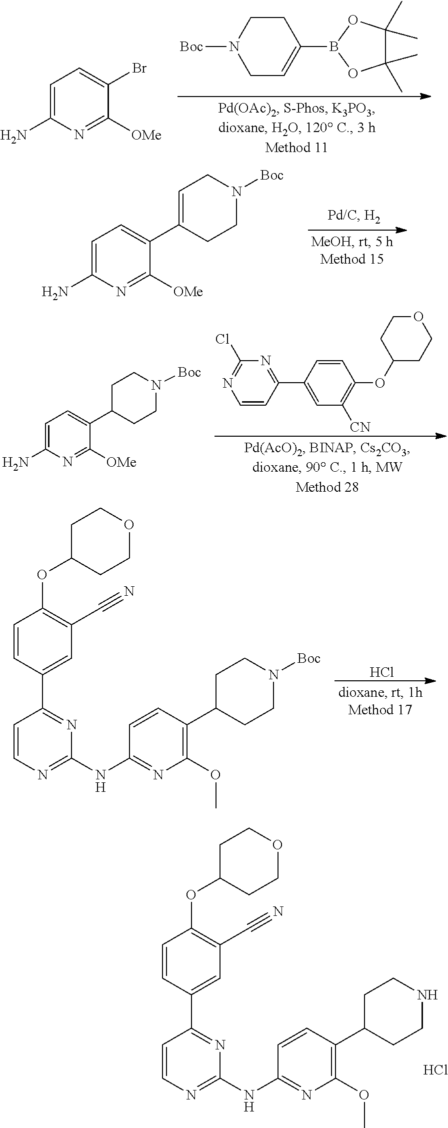

### Method 11

5-(2-[[6-methoxy-5-(piperidin-4-yl)pyridin-2-yl]amino]pyrimidin-4-yl)-2-(oxan-4-yloxy)benzonitrile hydrochloride: To a solution of 5-bromo-6-methoxypyridin-2-amine (475 mg, 2.34 mmol) in dioxane (6 mL) was added tert-butyl 4-(tetramethyl-1,3,2-dioxaborolan-2-yl)-1,2,3,6-tetrahydropyridine-1-carboxylate (1425 mg, 4.61 mmol), Pd(OAc)₂ (55 mg, 0.24 mmol), S-Phos (203 mg, 0.49 mmol) and K₃PO₄ solution (1570 mg in 2 mL water, 7.40 mmol) at room temperature. The resulting mixture was stirred for 3 h at 120° C. When the reaction was done, the reaction mixture was concentrated under reduced pressure and the residue was purified by flash chromatography eluting with EtOAc in hexane (0% to 60% gradient) to yield tert-butyl 4-(6-amino-2-methoxypyridin-3-yl)-1,2,3,6-tetrahydropyridine-1-carboxylate as an yellow oil (437 mg, 61%). MS: m/z=306.1 [M+H]⁺.

### Method 15

5-(2-[[6-methoxy-5-(piperidin-4-yl)pyridin-2-yl]amino]pyrimidin-4-yl)-2-(oxan-4-yloxy)benzonitrile hydrochloride: To a solution of tert-butyl 4-(6-amino-2-methoxypyridin-3-yl)-1,2,3,6-tetrahydropyridine-1-carboxylate (380 mg, 1.24 mmol) in MeOH (30 mL) was added palladium carbon (400 mg, 3.76 mmol) under nitrogen atmosphere. The reaction tank was vacuumed and flushed with hydrogen. Then the reaction mixture was hydrogenated for 5 h under hydrogen atmosphere using a hydrogen balloon at room temperature. When the reaction was done, the reaction mixture was Altered through a celite pad and the filtrate was concentrated under reduced pressure to yield tert-butyl 4-(6-amino-2-methoxypyridin-3-yl)piperidine-1-carboxylate as an yellow oil (368 mg, 96%). MS: m/z=307.9 [M+H]⁺.

5-(2-[[6-methoxy-5-(piperidin-4-yl)pyridin-2-yl]amino]pyrimidin-4-yl)-2-(oxan-4-yloxy)benzonitrile hydrochloride: 5-(2-[[6-methoxy-5-(piperidin-4-yl)pyridin-2-yl]amino]pyrimidin-4-yl)-2-(oxan-4-yloxy)benzonitrile hydrochloride was prepared from tert-butyl 4-(6-amino-2-methoxypyridin-3-yl)piperidine-1-carboxylate and 5-(2-chloropyrimidin-4-yl)-2-(tetrahydro-2H-pyran-4-yloxy)benzonitrile using Method 28 and 17a. The final product was purified by prep-HPLC under the following conditions: column, XBridge BEH C18 OBD Prep Column, 150×19 mm, 5 um; mobile phase, acetonitrile in water (with 0.05% HCl), 30% to 55% gradient in 8 min; detector, UV 254 nm. 5-(2-[[6-methoxy-5-(piperidin-4-yl)pyridin-2-yl]amino]pyrimidin-4-yl)-2-(oxan-4-yloxy)benzonitrile hydrochloride was obtained as a yellow solid (23 mg, 14% for 2 steps). HPLC: 92.3% purity, RT=1.38 min. MS: m/z=487.1 [M+H]⁺. ¹H NMR (300 MHz, Methanol-d₄) δ 8.72-8.61 (m, 2H), 8.61-8.51 (m, 1H), 7.83-7.68 (m, 2H), 7.52-7.42 (m, 1H), 7.13-7.04 (m, 1H), 5.02-4.91 (m, 1H), 4.13 (s, 3H), 4.04-3.90 (m, 2H), 3.72-3.58 (m, 2H), 3.55-3.44 (m, 2H), 3.21-3.07 (m, 3H), 2.18-1.73 (m, 8H).

### Example 40: 5-(2-[[6-methoxy-5-(1-methylpiperidin-4-yl)pyridin-2-yl]amino]pyrimidin-4-yl)-2-(oxan-4-yloxy)benzonitrile (40)

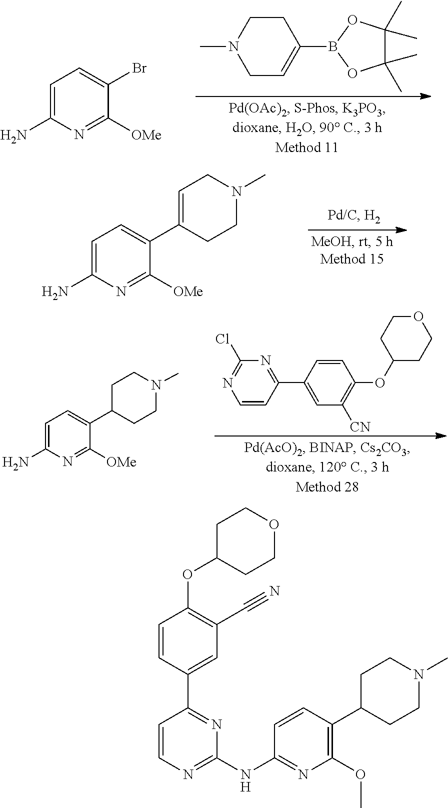

The title compound was prepared from 5-bromo-6-methoxypyridin-2-amine, 1-methyl-4-(4,4,5,5-tetramethyl-1,3,2-dioxaborolan-2-yl)-1,2,3,6-tetrahydropyridine and 5-(2-chloropyrimidin-4-yl)-2-(tetrahydro-2H-pyran-4-yloxy)benzonitrile using Method 11, 15, and 28. The final product was purified by reverse phase flash chromatography eluting with acetonitrile in water (with 10 mmol/L NH₄HCO₃), (0% to 50% gradient in 30 min). 5-(2-[[6-methoxy-5-(1-methylpiperidin-4-yl)pyridin-2-yl]amino]pyrimidin-4-yl)-2-(oxan-4-yloxy)benzonitrile was obtained as a light yellow solid (96 mg, 36% for 3 steps). HPLC: 97.9% purity, RT=1.80 min. MS: m/z=501.3 [M+H]⁺. ¹H NMR (300 MHz, DMSO-d₆) δ 9.46 (s, 1H), 8.64-8.55 (m, 2H), 8.53-8.43 (m, 1H), 7.84-7.75 (m, 1H), 7.60-7.50 (m, 3H), 5.00-4.92 (m, 1H), 3.92-3.82 (m, 5H), 3.62-3.49 (m, 2H), 2.91-2.80 (m, 2H), 2.67-2.61 (m, 1H), 2.18 (s, 3H), 2.10-1.87 (m, 4H), 1.77-1.51 (m, 6H).

### Example 41: 5-[2-[(6-methoxy-5-[[(1-methylpiperidin-4-yl)amino]methyl]pyridin-2-yl)amino]pyrimidin-4-yl]-2-(oxan-4-yloxy)benzonitrile hydrochloride (41)

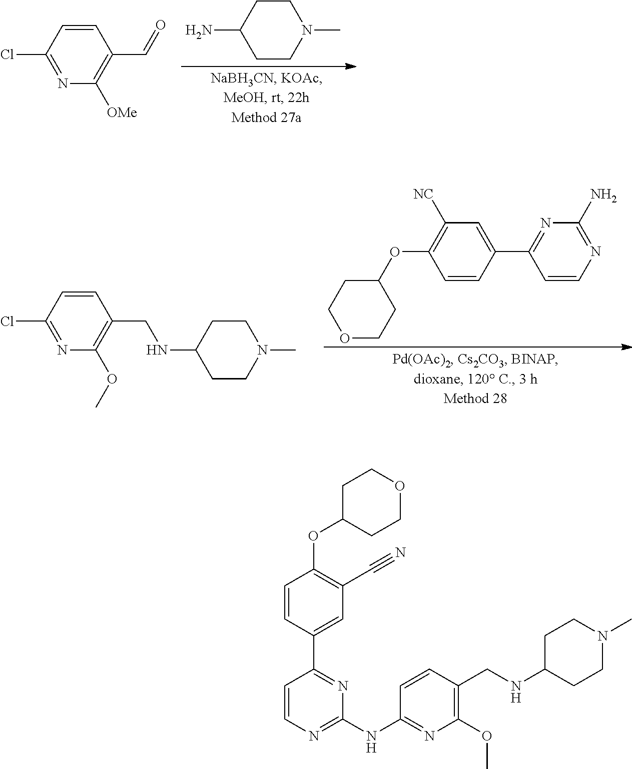

### Method 27a

N-[(6-chloro-2-methoxypyridin-3-yl)methyl]-1-methylpiperidin-4-amine: To a solution of 6-chloro-2-methoxypyridine-3-carbaldehyde (95 mg, 0.55 mmol) in MeOH (2 mL) was added 1-methylpiperidin-4-amine (63 mg, 0.55 mmol) at room temperature. The resulting solution was stirred for 30 min at room temperature and then was added by AcOH (0.03 mL, 0.50 mmol) and sodium boranecarbonitrile (35 mg, 0.56 mmol) in sequence at room temperature. The resulting mixture was stirred for 22 h at room temperature. When the reaction was done, the reaction mixture was quenched by sat. Na₂CO₃ solution (10 mL). The resulting mixture was extracted with dichloromethane (20 mL×3). The organic phases were combined, washed with brine and dried over Na₂SO₄. The solvent was removed under reduced pressure to yield N-[(6-chloro-2-methoxypyridin-3-yl)methyl]-1-methylpiperidin-4-amine as colorless oil (131 mg, 87%). MS: m/z=270.1 [M+H]⁺.

5-[2-[(6-methoxy-5-[[(1-methylpiperidin-4-yl)amino]methyl]pyridin-2-yl)amino]pyrimidin-4-yl]-2-(oxan-4-yloxy)benzonitrile hydrochloride: 5-[2-[(6-methoxy-5-[[(1-methylpiperidin-4-yl)amino]methyl]pyridin-2-yl)amino]pyrimidin-4-yl]-2-(oxan-4-yloxy)benzonitrile hydrochloride was prepared from N-[(6-chloro-2-methoxypyridin-3-yl)methyl]-1-methylpiperidin-4-amine and 5-(2-aminopyrimidin-4-yl)-2-(oxan-4-yloxy)benzonitrile using Method 28. The final product was purified by prep-HPLC under the following conditions: column, XBridge Prep C18 OBD Column, 150×19 mm, 5 um; mobile phase, acetonitrile in water (with 0.05% HCl), 30% to 60% gradient in 8 min; detector, UV 254 nm. 5-[2-[(6-methoxy-5-[[(1-methylpiperidin-4-yl)amino]methyl]pyridin-2-yl)amino]pyrimidin-4-yl]-2-(oxan-4-yloxy)benzonitrile hydrochloride was obtained as white solid (28 mg, 17). HPLC: 98.5% purity, RT=3.82 min. MS: m/z=530.3 [M+H]⁺. ¹H NMR (300 MHz, Methanol-d₄) δ 8.82-8.66 (m, 2H), 8.66-8.55 (m, 1H), 8.09-8.00 (m, 1H), 7.98-7.89 (m, 1H), 7.55-7.45 (m, 1H), 6.99 (d, J=8.0 Hz, 1H), 5.04-4.93 (m, 1H), 4.31 (s, 2H), 4.24 (s, 3H), 4.04-3.90 (m, 1H), 3.73-3.58 (m, 6H), 3.27-3.11 (m, 2H), 2.89 (s, 3H), 2.55-2.44 (m, 2H), 2.17-2.06 (m, 4H), 1.92-1.75 (m, 2H).

### Example 42: 5-(2-[[6-methoxy-5-(1-methylpyrrolidin-3-yl)pyridin-2-yl]amino]pyrimidin-4-yl)-2-(oxan-4-yloxy)benzonitrile (42)

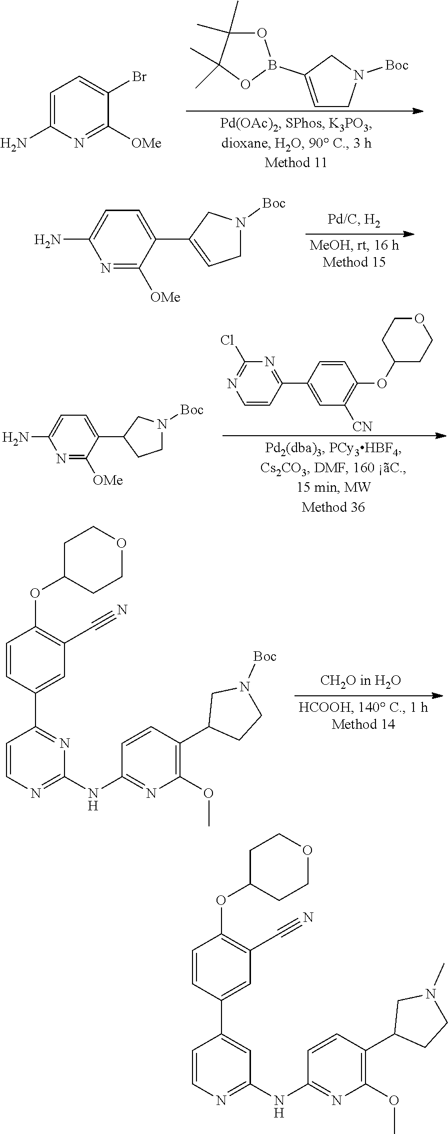

The title compound was prepared from 5-bromo-6-methoxypyridin-2-amine, tert-butyl 3-(4,4,5,5-tetramethyl-1,3,2-dioxaborolan-2-yl)-2H-pyrrole-1 (5H)-carboxylate, 5-(2-chloropyrimidin-4-yl)-2-(tetrahydro-2H-pyran-4-yloxy)benzonitrile, and formalin using Methods 11, 15, 36 and 14. The final product was purified by prep-HPLC under the following conditions: column, XBridge Prep C18 OBD Column, 150×19 mm, 5 um; mobile phase, acetonitrile in water (with 0.05% NH₃.H₂O), 30% to 50% gradient in 10 min; detector, UV 254 nm. 5-(2-[[6-methoxy-5-(1-methylpyrrolidin-3-yl)pyridin-2-yl]amino]pyrimidin-4-yl)-2-(oxan-4-yloxy)benzonitrile was obtained as a white solid (14 mg, 10% for 4 steps). HPLC: 94.6% purity, RT=3.00 min. MS: m/z=487.2 [M+H]⁺. ¹H NMR (300 MHz, Chloroform-d) δ 8.53 (d, J=5.2 Hz, 1H), 8.37-8.22 (m, 2H), 7.95-7.86 (m, 1H), 7.72 (s, 1H), 7.65-7.56 (m, 1H), 7.17-7.07 (m, 2H), 4.83-4.71 (m, 1H), 4.12-3.98 (m, 2H), 3.92 (s, 3H), 3.74-3.60 (m, 3H), 3.21-3.09 (m, 1H), 2.99-2.86 (m, 2H), 2.80-2.71 (m, 1H), 2.56 (s, 3H), 2.44-2.26 (m, 1H), 2.17-1.77 (m, 5H).

### Example 43: 5-(2-[[5-(3-fluoro-1-methylpiperidin-4-yl)-6-methoxypyridin-2-yl]amino]pyrimidin-4-yl)-2-(oxan-4-yloxy)benzonitrile (43)

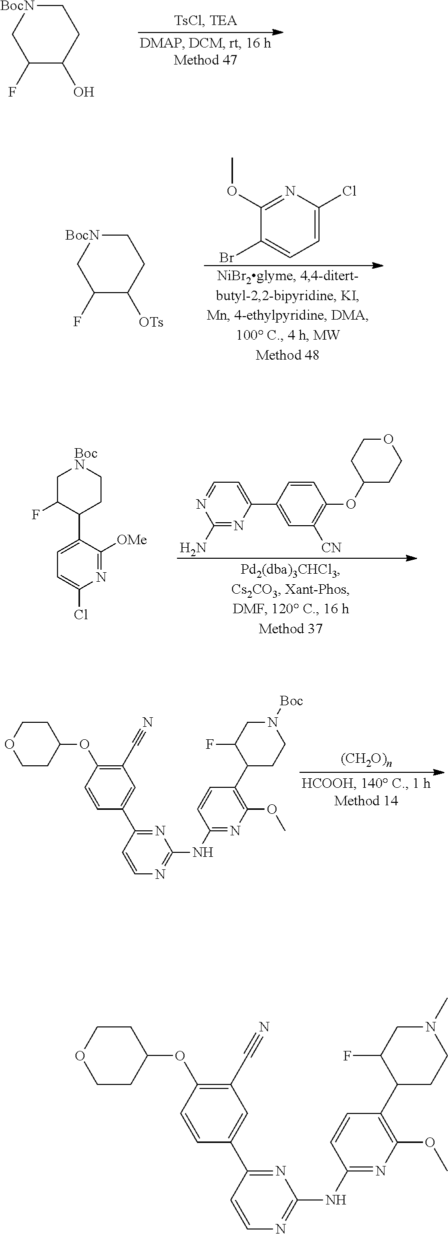

### Method 47

tert-butyl (3R,4S)-3-fluoro-4-[[(4-methylbenzene)sulfonyl]oxy]piperidine-1-carboxylate: At 0° C., to a solution of tert-butyl (3R,4S)-3-fluoro-4-hydroxypiperidine-1-carboxylate (180 mg, 0.87 mmol) in dichloromethane (8 mL) were added 4-dimethylaminopyridine (10 mg, 0.09 mmol), triethylamine (184 mg, 1.80 mmol). The resulting solution was then added by a solution of 4-methylbenzene-1-sulfonyl chloride in dichloromethane (0.25 M, 3.6 mL, 0.90 mmol) dropwise at 0° C. The resulting mixture was stirred for 16 h at room temperature. When the reaction was done, the reaction mixture was concentrated under reduced pressure and the residue was purified by flash chromatography eluting with EtOAc in hexane (0% to 30% gradient) to yield tert-butyl (3R,4S)-3-fluoro-4-[[(4-methylbenzene)sulfonyl]oxy]piperidine-1-carboxylate as off-white solid (94 mg, 29%).

### Method 48

tert-butyl 4-(6-chloro-2-methoxypyridin-3-yl)-3-fluoropiperidine-1-carboxylate: To a solution of 3-bromo-6-chloro-2-methoxypyridine (95 mg, 0.43 mmol) in DMA (7.5 mL) were added tert-butyl 3-fluoro-4-[[(4-methylbenzene)sulfonyl]oxy]piperidine-1-carboxylate (90 mg, 0.24 mmol), 4-tert-butyl-2-(4-tert-butylpyridin-2-yl)pyridine (25 mg, 0.09 mmol), 4-ethylpyridine (48 mg, 0.44 mmol), NiBr₂.glyme (29 mg, 0.09 mmol), KI (38 mg, 0.23 mmol) and Mn (38 mg, 0.69 mmol) at room temperature. The reaction mixture was irradiated with microwave for 4 h at 100° C. When the reaction was done, the insoluble solids in the reaction mixture were filtered out and the filtrate was concentrated under reduced pressure. The residue was purified by flash chromatography eluting with EtOAc in hexane (0% to 100% gradient) to yield 4-(6-chloro-2-methoxypyridin-3-yl)-3-fluoropiperidine-1-carboxylate as a light yellow solid (27 mg, 18%). MS: m/z=345.1 [M+H]⁺.

5-(2-[[5-(3-fluoro-1-methylpiperidin-4-yl)-6-methoxypyridin-2-yl]amino]pyrimidin-4-yl)-2-(oxan-4-yloxy)benzonitrile: 5-(2-[[5-(3-fluoro-1-methylpiperidin-4-yl)-6-methoxypyridin-2-yl]amino]pyrimidin-4-yl)-2-(oxan-4-yloxy)benzonitrile was prepared from tert-butyl 4-(6-chloro-2-methoxypyridin-3-yl)-3-fluoropiperidine-1-carboxylate, 5-(2-aminopyrimidin-4-yl)-2-(tetrahydro-2H-pyran-4-yloxy)benzonitrile and (HCHO)ₙ using Method 37 and 14. The final product was purified by prep-HPLC under the following conditions: column, XBridge Prep C18 OBD Column, 150×19 mm, 5 um; mobile phase, acetonitrile in water (with 10 mmol/L NR₄HCO₃ and 0.1% NH₃.H₂O), 5% to 52% gradient in 8 min; detector, UV 254 nm. 5-(2-[[5-(3-fluoro-1-methylpiperidin-4-yl)-6-methoxypyridin-2-yl]amino]pyrimidin-4-yl)-2-(oxan-4-yloxy)benzonitrile was obtained as an yellow solid (1.5 mg, 2.2% for 2 steps). HPLC: 96.8% purity, RT=4.77 min. MS: m/z=519.0 [M+H]⁺. ¹H NMR (300 MHz, DMSO-d₆) δ 9.55 (s, 1H), 8.65-8.55 (m, 2H), 8.54-8.43 (m, 1H), 7.87-7.77 (m, 1H), 7.75-7.65 (m, 1H), 7.61-7.51 (m, 2H), 5.03-4.69 (m, 2H), 3.93-3.82 (m, 5H), 3.61-3.50 (m, 2H), 3.24-3.14 (m, 1H), 3.00-2.65 (m, 2H), 2.26 (s, 3H), 2.07-1.92 (m, 4H), 1.72-1.65 (m, 4H).

### Example 44: 2-(2-[[6-methoxy-5-(4-methylpiperazin-1-yl)pyridin-2-yl]amino]pyrimidin-4-yl)-5-(oxan-4-yloxy)pyridine-4-carbonitrile hydrochloride (44)

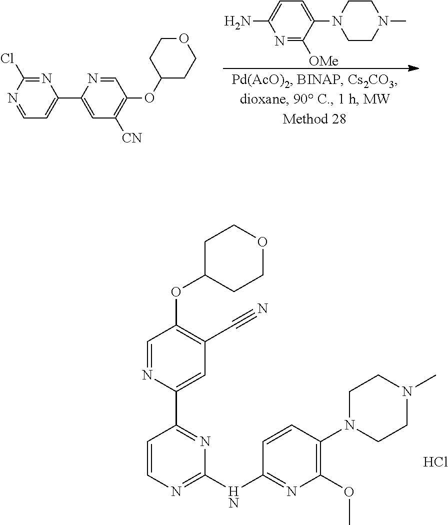 [SMILES: N#Cc1cc(-c2ccnc(Cl)n2)ncc1OC1CCOCC1 ; COc1nc(N)ccc1N1CCN(C)CC1 ; COc1nc(Nc2nccc(-c3cc(C#N)c(OC4CCOCC4)cn3)n2)ccc1N1CCN(C)CC1 ; Cl]

The title compound was prepared from 6-methoxy-5-(4-methylpiperazin-1-yl)pyridin-2-amine and 2-(2-chloropyrimidin-4-yl)-5-(tetrahydro-2H-pyran-4-yloxy)isonicotinonitrile using Method 28. The final product was purified by prep-HPLC under the following conditions: column, XBridge Prep Phenyl OBD Column, 150×19 mm, 5 um; mobile phase, acetonitrile in water (with 0.05% HCl), 10% to 40% gradient in 8 min; detector, UV 254 nm. 2-(2-[[6-methoxy-5-(4-methylpiperazin-1-yl)pyridin-2-yl]amino]pyrimidin-4-yl)-5-(oxan-4-yloxy)pyridine-4-carbonitrile hydrochloride was obtained as orange solid (9 mg, 8%). HPLC: 92.8% purity, RT=2.17 min. MS: m/z=503.2 [M+H]⁺. ¹H NMR (300 MHz, DMSO-d₆) δ 8.98 (s, 1H), 8.73 (d, J=5.3 Hz, 1H), 8.63 (s, 1H), 7.73 (d, J=5.3 Hz, 1H), 7.68-7.62 (m, 1H), 7.47-7.37 (m, 1H), 5.23-5.11 (m, 1H), 4.00-3.81 (m, 5H), 3.64-3.44 (m, 6H), 3.29-3.14 (m, 2H), 3.14-2.99 (m, 2H), 2.82 (s, 3H), 2.15-2.02 (m, 2H), 1.82-1.64 (m, 2H).

### Example 45: 2-(2-[[6-methoxy-5-(1-methylpiperidin-4-yl)pyridin-2-yl]amino]pyrimidin-4-yl)-5-(oxan-4-yloxy)pyridine-4-carbonitrile (45)

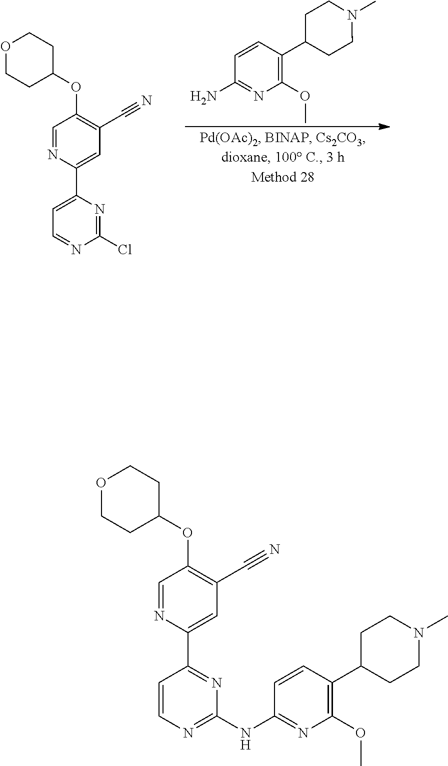 [SMILES: N#Cc1cc(-c2ccnc(Cl)n2)ncc1OC1CCOCC1 ; COc1nc(N)ccc1C1CCN(C)CC1 ; COc1nc(Nc2nccc(-c3cc(C#N)c(OC4CCOCC4)cn3)n2)ccc1C1CCN(C)CC1]

The title compound was prepared from 2-(2-chloropyrimidin-4-yl)-5-(oxan-4-yloxy)pyridine-4-carbonitrile and 6-methoxy-5-(1-methylpiperidin-4-yl)pyridin-2-amine using Method 28. The final product was purified by prep-HPLC under the following conditions: column, XBridge Shield RP18 OBD Column, 150×19 mm, 5 um; mobile phase, acetonitrile in water (with 10 mmol/L NH₄HCO₃), 35% to 65% gradient in 8 min; detector, UV 254 nm. 2-(2-[[6-methoxy-5-(1-methylpiperidin-4-yl)pyridin-2-yl]amino]pyrimidin-4-yl)-5-(oxan-4-yloxy)pyridine-4-carbonitrile was obtained as a light yellow solid (53 mg, 37%). HPLC: 98.9% purity, RT=1.83 min. MS: m/z=502.2 [M+H]⁺. ¹H NMR (300 MHz, DMSO-d₆) δ 9.59 (s, 1H), 8.96 (s, 1H), 8.74-8.60 (m, 2H), 7.87-7.77 (m, 1H), 7.68 (d, J=5.1 Hz, 1H), 7.62-7.53 (m, 1H), 5.20-5.08 (m, 1H), 3.94-3.82 (m, 5H), 3.63-3.49 (m, 2H), 3.19-3.09 (m, 2H), 2.83-2.72 (m, 1H), 2.50-2.43 (m, 5H), 2.15-2.04 (m, 2H), 1.87-1.64 (m, 6H).

### Example 46: 6-(2-[[6-methoxy-5-(1-methylpiperidin-4-yl)pyridin-2-yl]amino]pyrimidin-4-yl)-3-(oxan-4-yloxy)pyridine-2-carbonitrile (46)

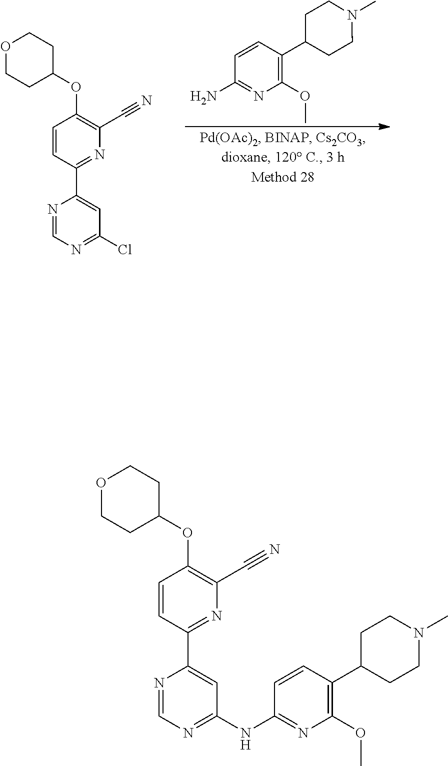 [SMILES: N#Cc1nc(-c2cc(Cl)ncn2)ccc1OC1CCOCC1 ; COc1nc(N)ccc1C1CCN(C)CC1 ; COc1nc(Nc2cc(-c3ccc(OC4CCOCC4)c(C#N)n3)ncn2)ccc1C1CCN(C)CC1]

The title compound was prepared from 6-(2-chloropyrimidin-4-yl)-3-(oxan-4-yloxy)pyridine-2-carbonitrile and 6-methoxy-5-(1-methylpiperidin-4-yl)pyridin-2-amine using Method 28. The final product was purified by prep-HPLC under the following conditions: column, XBridge Prep C18 OBD Column, 150×19 mm, 5 um; mobile phase, acetonitrile in water (with 10 mmol/L NR₄HCO₃ and 0.1% NH₃.H₂O), 59% to 59% gradient in 8 min; detector, UV 254 nm. 6-(2-[[6-methoxy-5-(1-methylpiperidin-4-yl)pyridin-2-yl]amino]pyrimidin-4-yl)-3-(oxan-4-yloxy)pyridine-2-carbonitrile was obtained as an yellow solid (29 mg, 28%). HPLC: 96.1% purity, RT=1.00 min. MS: m/z=502.2 [M+H]⁺. ¹H NMR (400 MHz, Chloroform-d) δ 8.68-8.60 (m, 2H), 7.94-7.87 (m, 1H), 7.85-7.72 (m, 2H), 7.59-7.51 (m, 2H), 4.85-4.75 (m, 1 H), 4.13-4.02 (m, 2H), 3.94 (s, 3H), 3.75-3.64 (m, 2H), 3.12-3.04 (m, 2H), 2.87-2.77 (m, 1H), 2.42 (s, 3H), 2.31-1.72 (m, 10H).

### Example 47: 5-([4-[3-cyano-4-(oxan-4-yloxy)phenyl]pyrimidin-2-yl]amino)-4-methoxy-N,N-dimethylpyridine-2-carboxamide (47)

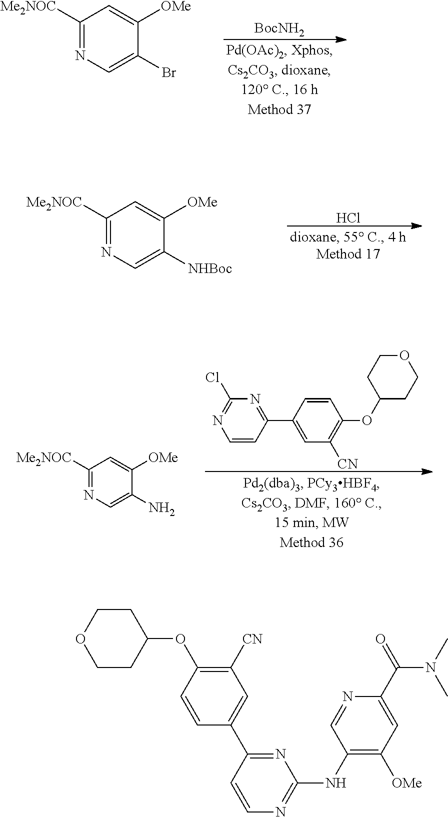

The title compound was prepared from 2-(tetrahydro-2H-pyran-4-ylamino)-5-(4,4,5,5-tetramethyl-1,3,2-dioxaborolan-2-yl)benzonitrile, 2,4-dichloropyrimidine, and 6-amino-5-methoxy-N,N-dimethylnicotinamide using Method 37, 17 and 36. The final product was purified by prep-HPLC under the following conditions: column, XBridge Prep C18 Column, 150×19 mm, 5 um; mobile phase, acetonitrile in water (with 10 mmol/L NH₄HCO₃), 25% to 60% gradient in 8 min; detector, UV 254 nm. 5-([4-[3-cyano-4-(oxan-4-yloxy)phenyl]pyrimidin-2-yl]amino)-4-methoxy-N,N-dimethylpyridine-2-carboxamide was obtained as a white solid (13 mg, 7.4% for 3 steps). HPLC: 93.1% purity, RT=1.18 min. MS: m/z=475.0 [M+H]⁺. ¹H NMR (300 MHz, Methanol-d₄) δ 9.58 (s, 1H), 8.60-8.52 (m, 1H), 8.50-8.38 (m, 2H), 7.47-7.31 (m, 3H), 5.00-4.90 (m, 1H), 4.10 (s, 3H), 4.05-3.90 (m, 2H), 3.76-3.62 (m, 2H), 3.16 (s, 3H), 3.12 (s, 3H), 2.21-2.07 (m, 2H), 1.96-1.78 (m, 2H).

### Example 48: 5-([4-[3-cyano-4-(oxan-4-yloxy)phenyl]pyrimidin-2-yl]amino)-6-methoxy-N,N-dimethylpyridine-2-carboxamide (48)

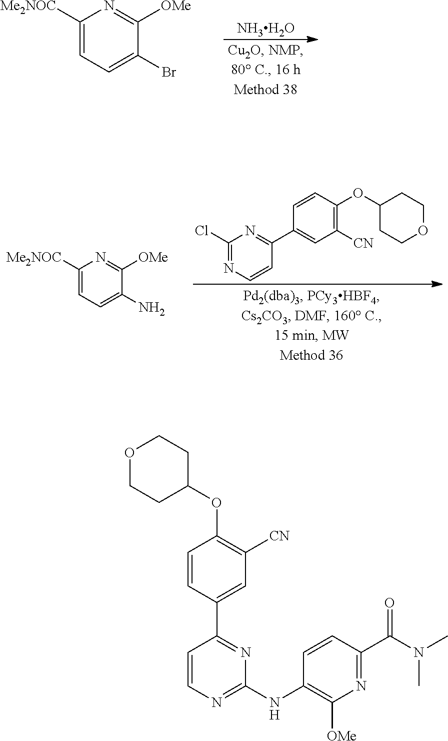

The title compound was prepared from 5-bromo-6-methoxy-N,N-dimethylpicolinamide, 5-amino-6-methoxy-N,N-dimethylpicolinamide and 5-(2-chloropyrimidin-4-yl)-2-(tetrahydro-2H-pyran-4-yloxy)benzonitrile using Method 38 and 36. The final product was purified by prep-HPLC under the following conditions: column, XBridge Prep C18 Column, 150×19 mm, 5 um; mobile phase, acetonitrile in water (with 10 mmol/L NH₄HCO₃), 20% to 60% gradient in 8 min; detector, UV 254 nm. 5-([4-[3-cyano-4-(oxan-4-yloxy)phenyl]pyrimidin-2-yl]amino)-6-methoxy-N,N-dimethylpyridine-2-carboxamide was obtained as a white solid (10 mg, 4.4% for 2 steps). HPLC: 99.9% purity, RT=1.52 min. MS: m/z=475.2 [M+H]⁺. ¹H NMR (300 MHz, DMSO-d₆) δ 8.69-8.37 (m, 5H), 7.62-7.48 (m, 2H), 7.32 (d, J=8.0 Hz, 1H), 4.99-4.90 (m, 1H), 3.98 (s, 3H), 3.94-3.81 (m, 2H), 3.63-3.49 (m, 2H), 3.12 (s, 3H), 3.01 (s, 3H), 2.11-1.99 (m, 2H), 1.78-1.60 (m, 2H).

### Example 49: 6-([4-[3-cyano-4-(oxan-4-yloxy)phenyl]pyrimidin-2-yl]amino)-4-methoxy-N,N-dimethylpyridine-3-carboxamide (49)

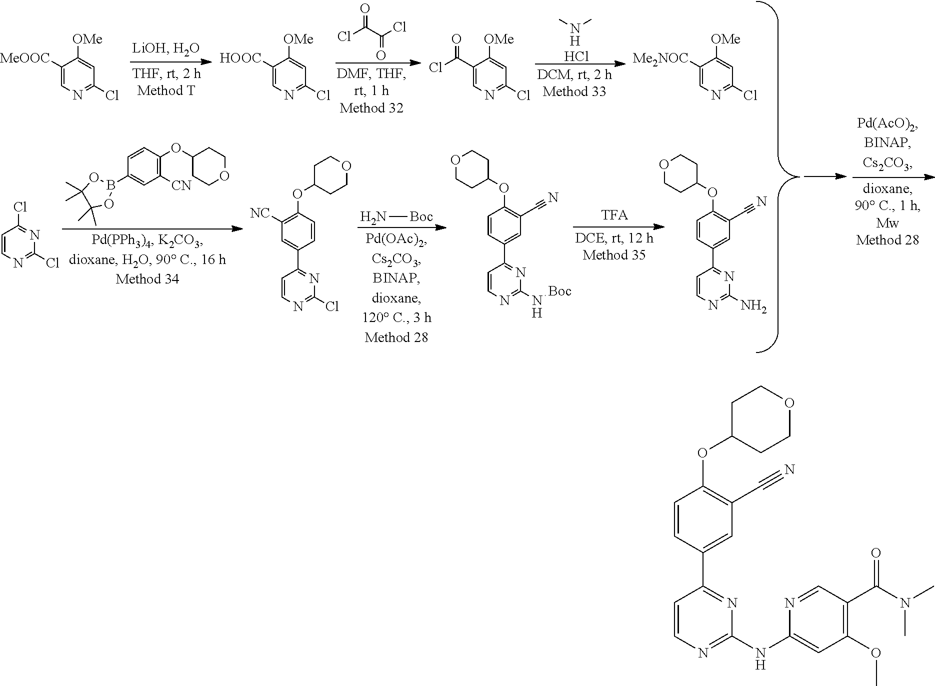

### Method T

6-Chloro-4-methoxypyridine-3-carboxylic acid: To a solution of methyl 6-chloro-4-methoxypyridine-3-carboxylate (804 mg, 3.99 mmol) in THF (10 mL) was added a solution of LiOH (299 mg, 12.50 mmol) in water (2.5 mL) at room temperature. The resulting mixture was stirred for 2 h at room temperature. When the reaction was done, the pH value of the reaction mixture was adjusted to 2-3 with hydrogen chloride solution (2 mol/L). The resulting mixture was concentrated under reduced pressure and the remaining solution was extracted with ethyl acetate (50 mL×3). The organic phases were combined, washed with brine and dried over Na₂SO₄. The solvent was removed under reduced pressure to yield 6-chloro-4-methoxypyridine-3-carboxylic acid as off-white solid (770 mg, 90%). MS: m/z=188.1 [M+H]⁺.

### Method 32

6-Chloro-4-methoxypyridine-3-carbonyl chloride: At 0° C., to a solution of 6-bromo-4-methoxypyridine-3-carboxylic acid (670 mg, 2.85 mmol) in THF (8 mL) was added N,N-dimethylformamide (0.2 mL) and oxalic dichloride (2.81 g, 22.30 mmol) in sequence. The resulting mixture was stirred for 1 h at room temperature. When the reaction was done, the reaction mixture was concentrated under reduced pressure to yield 6-chloro-4-methoxypyridine-3-carbonyl chloride as an yellow solid (850 mg, crude).

### Method 33

6-chloro-4-methoxy-N,N-dimethylpyridine-3-carboxamide: To a solution of dimethylamine hydrochloride (485 mg, 5.94 mmol) in dichloromethane (10 mL) was added DIE A (2 mL) and 6-chloro-4-methoxypyridine-3-carbonyl chloride (850 mg, crude) at room temperature. The resulting mixture was stirred for 2 h at room temperature. When the reaction was done, the reaction mixture was concentrated under reduced pressure. The residue was purified by flash chromatography eluting with EtOAc in hexane (0% to 70% gradient) to yield 6-chloro-4-methoxy-N,N-dimethylpyridine-3-carboxamide as an yellow oil (706 mg, 92% for 2 steps). MS: m/z=215.2 [M+H]⁺.

### Method 34

5-(2-chloropyrimidin-4-yl)-2-(oxan-4-yloxy)benzonitrile): To a solution of 2,4-dichloropyrimidine (3.00 g, 20.14 mmol) in dioxane (30 mL) was added 2-(oxan-4-yloxy)-5-(tetramethyl-1,3,2-dioxaborolan-2-yl)benzonitrile (6.60 g, 20.05 mmol), Pd(PPh₃)₄ (400 mg, 0.35 mmol) and potassium carbonate solution (5.40 g in 9 mL water, 39.07 mmol) at room temperature. The resulting mixture was stirred for 16 h at 90° C. After cooling to room temperature, the reaction mixture was diluted by EtOAc (150 mL). The organic phases were washed with brine and dried over Na₂SO₄. The solvent was removed under reduced pressure and the residue was purified by flash chromatography eluting with EtOAc in hexane (0% to 66% gradient) to yield 5-(2-chloropyrimidin-4-yl)-2-(oxan-4-yloxy)benzonitrile as gray solid (2.80 g, 44%). MS: m/z=316.0 [M+H]⁺.

### Method 28

tert-Butyl N-[4-[3-cyano-4-(oxan-4-yloxy)phenyl]pyrimidin-2-yl]carbamate: To a solution of 5-(2-chloropyrimidin-4-yl)-2-(oxan-4-yloxy)benzonitrile (930 mg, 3.0 mmol) in dioxane (25 mL) was added tert-butyl carbamate (450 mg, 3.8 mmol), Pd(OAc)₂ (70 mg, 0.3 mmol), BINAP (590 mg, 0.9 mmol) and Cs₂CO₃ (1550 mg, 4.8 mmol) at room temperature. The resulting mixture was stirred for 3 h at 120° C. After cooling to room temperature, the reaction mixture was concentrated under reduced pressure and the residue was purified by flash chromatography eluting with EtOAc in hexane (0% to 66% gradient) to yield tert-butyl N-[4-[3-cyano-4-(oxan-4-yloxy)phenyl]pyrimidin-2-yl]carbamate as an yellow solid (680 mg, 58%). MS: m/z=397.3 [M+H]⁺.

### Method 35

5-(2-aminopyrimidin-4-yl)-2-(oxan-4-yloxy)benzonitrile: To a solution of tert-butyl N-[4-[3-cyano-4-(oxan-4-yloxy)phenyl]pyrimidin-2-yl]carbamate (500 mg, 0.99 mmol) in DCE (24 mL) was added TFA (8.90 g, 91.55 mmol) at room temperature. The resulting solution was stirred for 12 h at room temperature. When the reaction was done, the resulting mixture was concentrated under reduced pressure to yield 5-(2-aminopyrimidin-4-yl)-2-(oxan-4-yloxy)benzonitrile as an yellow solid (350 mg, crude).

6-([4-[3-cyano-4-(oxan-4-yloxy)phenyl]pyrimidin-2-yl]amino)-4-methoxy-N,N-dimethylpyridine-3-carboxamide: 6-([4-[3-cyano-4-(oxan-4-yloxy)phenyl]pyrimidin-2-yl]amino)-4-methoxy-N,N-dimethylpyridine-3-carboxamide was prepared from 5-(2-aminopyrimidin-4-yl)-2-(oxan-4-yloxy)benzonitrile and 6-chloro-4-methoxy-N,N-dimethylpyridine-3-carboxamide using Method 28. The final product was purified by prep-HPLC under the following conditions: column, XBridge Prep C18 OBD Column, 19×150 mm 5 um; mobile phase, acetonitrile in water (with 10 mmol/L NH₄HCO₃), 30% to 55% gradient in 8 min; detector, UV 254/220 nm. 6-([4-[3-cyano-4-(oxan-4-yloxy)phenyl]pyrimidin-2-yl]amino)-4-methoxy-N,N-dimethylpyridine-3-carboxamide was obtained as a white solid (20 mg, 12% for 2 steps). HPLC: 95.0% purity, RT=1.31 min. MS: m/z=475.1 [M+H]⁺. ¹H NMR (300 MHz, DMSO-d6) δ 10.07 (s, 1H), 8.69-8.58 (m, 2H), 8.52-8.42 (m, 1H), 8.21 (s, 1H), 8.00 (s, 1H), 7.65-7.49 (m, 2H), 4.98-4.91 (m, 1H), 3.98 (s, 3H), 3.91-3.80 (m, 2H), 3.58-3.51 (m, 2H), 2.97 (s, 3H), 2.83 (s, 3H), 2.04-1.98 (m, 2H), 1.73-1.62 (m, 2H).

### Example 50: 5-([4-[3-cyano-4-(oxan-4-yloxy)phenyl]pyrimidin-2-yl]amino)-3-methoxy-N,N-dimethylpyridine-2-carboxamide (50)

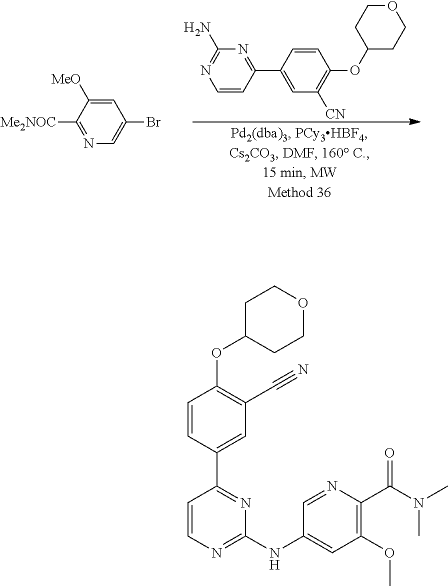

### Method 36

5-([4-[3-cyano-4-(oxan-4-yloxy)phenyl]pyrimidin-2-yl]amino)-3-methoxy-N,N-dimethylpyridine-2-carboxamide: To a solution of 5-bromo-3-methoxy-N,N-dimethylpyridine-2-carboxamide (100 mg, 0.39 mmol) in DMF (10 mL) was added 5-(2-aminopyrimidin-4-yl)-2-(oxan-4-yloxy)benzonitrile (280 mg, 0.94 mmol), Cs₂CO₃ (377 mg, 1.16 mmol), PCy₃.HBF₄ (85 mg, 0.23 mmol) and Pd₂(dba)₃.CHCl₃ (80 mg, 0.08 mmol) at room temperature. The reaction mixture was irradiated with microwave for 15 min at 160° C. When the reaction was done, the resulting mixture was concentrated under reduced pressure and the residue was purified by prep-HPLC under the following conditions: column, XBridge Prep C18 OBD Column, 19×150 mm 5 um; mobile phase, acetonitrile in water (with 10 mmol/L NH₄HCO₃), 30% to 60% gradient in 8 min; detector, UV 254/220 nm. 5-([4-[3-cyano-4-(oxan-4-yloxy)phenyl]pyrimidin-2-yl]amino)-3-methoxy-N,N-dimethylpyridine-2-carboxamide was obtained as a white solid (15 mg, 8%). HPLC: 99.6% purity, RT=2.45 min. MS: m/z=475.2 [M+H]⁺. ¹H NMR (400 MHz, DMSO-d₆) δ 10.07 (s, 1H), 8.65-8.54 (m, 2H), 8.50-8.40 (m, 2H), 8.27-8.21 (m, 1H), 7.59-7.51 (m, 2H), 4.98-4.89 (m, 1H), 3.88-3.84 (m, 5H), 3.59-3.51 (m, 2H), 2.96 (s, 3H), 2.74 (s, 3H), 2.06-1.98 (m, 2H), 1.74-1.60 (m, 2H).

### Example 51: 6-([4-[4-cyano-5-(oxan-4-yloxy)pyridin-2-yl]pyrimidin-2-yl]amino)-4-methoxy-N,N-dimethylpyridine-3-carboxamide (51)

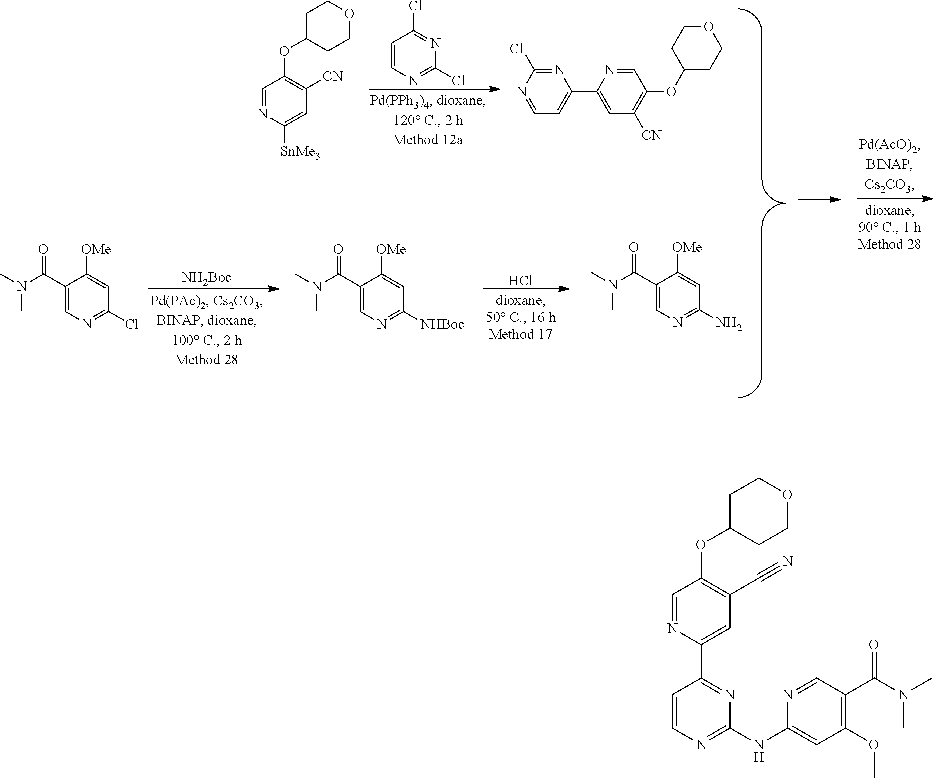

### Method 12a

2-(2-chloropyrimidin-4-yl)-5-(oxan-4-yloxy)pyridine-4-carbonitrile: To a solution of 5-(oxan-4-yloxy)-2-(trimethylstannyl)pyridine-4-carbonitrile (540 mg, 1.47 mmol) in dioxane (8 mL) were added 2,4-dichloropyrimidine (230 mg, 1.54 mmol) and Pd(PPh₃)₄ (255.02 mg, 0.22 mmol) at room temperature. The resulting mixture was stirred for 2 h at 120° C. After cooling to room temperature, the reaction mixture was concentrated under reduced pressure and the residue was purified by flash chromatography eluting with EtOAc in petroleum ether (0% to 70% gradient) to yield 2-(2-chloropyrimidin-4-yl)-5-(oxan-4-yloxy)pyridine-4-carbonitrile as a light yellow solid (349 mg, 75%). MS: m/z=317.2 [M+H]⁺.

6-([4-[4-cyano-5-(oxan-4-yloxy)pyridin-2-yl]pyrimidin-2-yl]amino)-4-methoxy-N,N-dimethylpyridine-3-carboxamide: tert-butyl N-[5-(dimethylcarbamoyl)-4-methoxypyridin-2-yl]carbamate was prepared from 6-chloro-4-methoxy-N,N-dimethylpyridine-3-carboxamide and tert-butyl carbamate using Method 28. The final product was purified by flash chromatography eluting with EtOAc in hexane (0% to 70% gradient) to yield tert-butyl N-[5-(dimethylcarbamoyl)-4-methoxypyridin-2-yl]carbamate as an yellow solid (570 mg, 60%). MS: m/z=296.3 [M+H]⁺.

### Method 17

6-([4-[4-cyano-5-(oxan-4-yloxy)pyridin-2-yl]pyrimidin-2-yl]amino)-4-methoxy-N,N-dimethylpyridine-3-carboxamide: At room temperature, tert-butyl N-[5-(dimethylcarbamoyl)-4-methoxypyridin-2-yl]carbamate (570 mg, 1.93 mmol) was added to a solution of hydrogen chloride in dioxane (6 M, 3.2 mL, 19.3 mmol). The resulting solution was stirred for 16 h at 50° C. When the reaction was done, the reaction mixture was concentrated under reduced pressure to yield 6-amino-4-methoxy-N,N-dimethylpyridine-3-carboxamide as an yellow solid (700 mg, crude). MS: m/z=196.0 [M+H]⁺.

6-([4-[4-cyano-5-(oxan-4-yloxy)pyridin-2-yl]pyrimidin-2-yl]amino)-4-methoxy-N,N-dimethylpyridine-3-carboxamide: The title compound was prepared from 2-(2-chloropyrimidin-4-yl)-5-(tetrahydro-2H-pyran-4-yloxy)isonicotinonitrile and 6-amino-4-methoxy-N,N-dimethylnicotinamide using Method 28. The final product was purified by prep-HPLC under the following conditions: column, XBridge Prep C18 OBD Column, 150 mm 5 um; mobile phase, acetonitrile in water (with 10 mmol/L NH₄HCO₃), 30% to 55% gradient in 8 min; detector, UV 254 nm. 6-([4-[4-cyano-5-(oxan-4-yloxy)pyridin-2-yl]pyrimidin-2-yl]amino)-4-methoxy-N,N-dimethylpyridine-3-carboxamide was obtained as a light yellow solid (35 mg, 23%). HPLC: 96.7% purity, RT=0.94 min. MS: m/z=476.2 [M+H]⁺. ¹H NMR (400 MHz, DMSO-de) δ 10.20 (s, 1H), 8.98 (s, 1H), 8.80-8.69 (m, 2H), 8.27 (s, 1H), 8.04 (s, 1H), 7.79-7.73 (m, 1H), 5.19-5.13 (m, 1H), 4.05 (s, 3H), 3.93-3.85 (m, 2H), 3.62-3.55 (m, 2H), 2.99 (s, 3H), 2.86 (s, 3H), 2.15-2.06 (m, 2H), 1.77-1.71 (m, 2H).

### Example 52: 5-([4-[4-cyano-5-(oxan-4-yloxy)pyridin-2-yl]pyrimidin-2-yl]amino)-3-methoxy-N,N-dimethylpyridine-2-carboxamide (52)

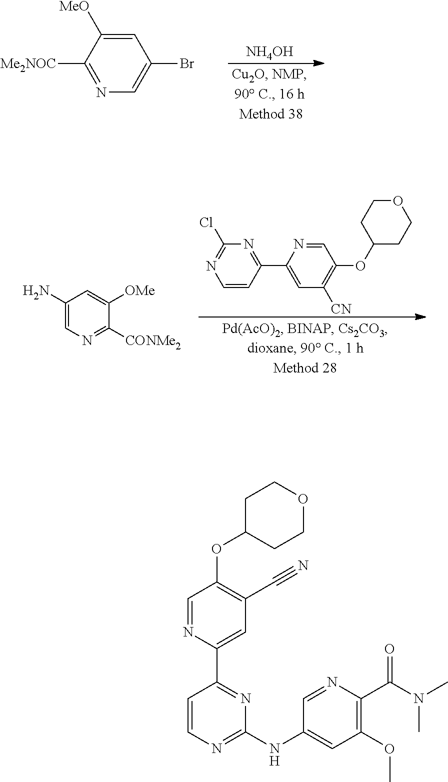

### Method 38

5-amino-3-methoxy-N,N-dimethylpyridine-2-carboxamide: To a solution of 5-bromo-3-methoxy-N,N-dimethylpyridine-2-carboxamide (164 mg, 0.63 mmol) in NMP (2 mL) was added ammonia solution (30% in water, 2 mL, 15.41 mmol), Cu₂O (19 mg, 0.13 mmol) at room temperature. The resulting mixture was stirred for 16 h at 90° C. When the reaction was done, the reaction mixture was diluted by H₂O (10 mL), and was extracted with ethyl acetate (20 mL×3). The organic phases were combined, washed with brine and dried over Na₂SO₄. The solvent was removed under reduced pressure to yield 5-amino-3-methoxy-N,N-dimethylpyridine-2-carboxamide as an yellow oil (150 mg, crude).

5-([4-[4-cyano-5-(oxan-4-yloxy)pyridin-2-yl]pyrimidin-2-yl]amino)-3-methoxy-N,N-dimethylpyridine-2-carboxamide: The title compound was prepared from 5-amino-3-methoxy-N,N-dimethylpicolinamide and 2-(2-chloropyrimidin-4-yl)-5-(tetrahydro-2H-pyran-4-yloxy)isonicotinonitrile using Method 28. The final product was purified by prep-HPLC under the following conditions: column, SunFire Prep C18 OBD Column, 150×19 mm, 5 um; mobile phase, acetonitrile in water (with 10 mmol/L NH₄HCO₃), 30% to 55% gradient in 8 min; detector, UV 254 nm. 5-([4-[4-cyano-5-(oxan-4-yloxy)pyridin-2-yl]pyrimidin-2-yl]amino)-3-methoxy-N,N-dimethylpyridine-2-carboxamide was obtained as a white solid (6 mg, 6%). HPLC: 99.6% purity, RT=3.20 min. MS: m/z=476.1 [M+H]⁺. ¹H NMR (300 MHz, Methanol-d₄) δ 8.75 (s, 1H), 8.6-8.57 (m, 2H), 8.47-8.36 (m, 2H), 7.81-7.72 (m, 1H), 5.11-5.02 (m, 1H), 4.05-3.91 (m, 5H), 3.72-3.58 (m, 2H), 3.10 (s, 3H), 2.90 (s, 3H), 2.20-2.08 (m, 2H), 1.85 (m, 2H).

### Example 53: 5-(2-[[4-methoxy-5-(4-methylpiperazin-1-yl)pyridin-2-yl]amino]pyrimidin-4-yl)-2-(oxan-4-yloxy)benzonitrile hydrochloride (53)

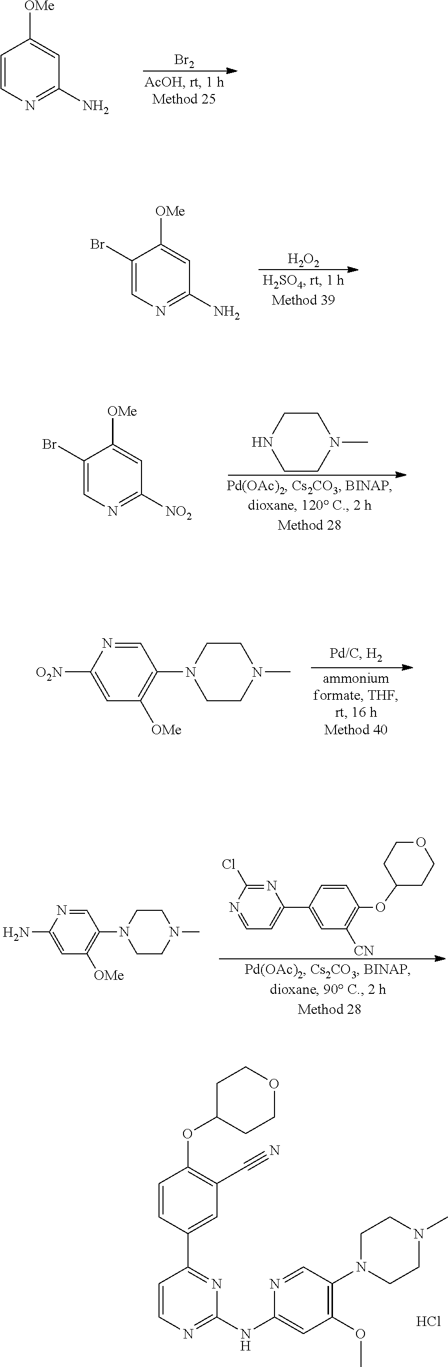

5-bromo-4-methoxypyridin-2-amine: 5-bromo-4-methoxypyridin-2-amine was prepared from 4-methoxypyridin-2-amine and bromine using Method 25. The product was purified by flash chromatography eluting with EtOAc in hexane (30% to 70% gradient) to yield 5-bromo-4-methoxypyridin-2-amine as a white solid (3.15 g, 41%). MS: m/z=203.0 [M+H]⁺.

### Method 39

5-amino-3-methoxy-N,N-dimethylpyridine-2-carboxamide: At 0° C., to a solution of 5-bromo-4-methoxypyridin-2-amine (2.98 g, 14.65 mmol) in sulfuric acid (36 mL) was added dropwise a solution of H₂O₂ (32 mL, 1.30 mol, 30%) in sulfuric acid (36 mL). The resulting mixture was stirred for 10 min at 0° C., warmed up to room temperature, and stirred for additional 1 h at room temperature. When the reaction was done, the pH value of the reaction mixture was adjusted to 7-8 with sat. sodium carbonate solution. The resulting mixture was extracted with ethyl acetate (500 mL×3). The organic phases were combined, washed with brine and dried over Na₂SO₄. The solvent was removed under reduced pressure and the residue was purified by flash chromatography eluting with EtOAc in hexane (0% to 95% gradient) to yield 5-bromo-4-methoxy-2-nitropyridine as a white solid (801 mg, 25%). MS: m/z=234.6 [M+H]⁺.

1-(4-methoxy-6-nitropyridin-3-yl)-4-methylpiperazine: 1-(4-methoxy-6-nitropyridin-3-yl)-4-methylpiperazine was prepared from 5-bromo-4-methoxy-2-nitropyridine and 1-methylpiperazine using Method 28. The product was purified by flash chromatography eluting with MeOH in EtOAc (0% to 20% gradient) to yield 1-(4-methoxy-6-nitropyridin-3-yl)-4-methylpiperazine as an yellow solid (326 mg, 63%). MS: m/z=253.1 [M+H]⁺.

### Method 40

4-methoxy-5-(4-methylpiperazin-1-yl)pyridin-2-amine: To a solution of 1-(4-methoxy-6-nitropyridin-3-yl)-4-methylpiperazine (653 mg, 2.59 mmol) in THF (15 mL) was added ammonium formate (1.14 g, 18.08 mmol) and palladium carbon (57 mg, 0.54 mmol) under nitrogen atmosphere. The reaction tank was vacuumed and flushed with hydrogen. Then the reaction mixture was hydrogenated for 15 h at room temperature under hydrogen atmosphere using a hydrogen balloon. When the reaction was done, the reaction mixture was filtered through a celite pad and the filtrate was concentrated under reduced pressure to yield 4-methoxy-5-(4-methylpiperazin-1-yl)pyridin-2-amine as an yellow solid (400 mg, 70%). MS: m/z=223.0 [M+H]⁺.

5-(2-[[4-methoxy-5-(4-methylpiperazin-1-yl)pyridin-2-yl]amino]pyrimidin-4-yl)-2-(oxan-4-yloxy)benzonitrile hydrochloride: 5-(2-[[4-methoxy-5-(4-methylpiperazin-1-yl)pyridin-2-yl]amino]pyrimidin-4-yl)-2-(oxan-4-yloxy)benzonitrile hydrochloride was prepared from 5-(2-chloropyrimidin-4-yl)-2-(tetrahydro-2H-pyran-4-yloxy)benzonitrile and 4-methoxy-5-(4-methylpiperazin-1-yl)pyridin-2-amine using Method 28. The final product was purified by prep-HPLC under the following conditions: column, XBridge Prep C18 OBD Column, 150×19 mm, 5 um; mobile phase, acetonitrile in water (with 0.05% HCl), 20% to 50% gradient in 8 min; detector, UV 254 nm. 5-(2-[[4-methoxy-5-(4-methylpiperazin-1-yl)pyridin-2-yl]amino]pyrimidin-4-yl)-2-(oxan-4-yloxy)benzonitrile hydrochloride was obtained as gray solid (6 mg, 5%). HPLC: 96.4% purity, RT=8.25 min. MS: m/z=502.3 [M+H]⁺. ¹H NMR (300 MHz, Methanol-d₄) δ 8.77-8.68 (m, 1H), 8.59-8.52 (m, 1H), 8.51-8.41 (m, 1H), 7.86 (s, 1H), 7.75 (d, J=5.4 Hz, 1H), 7.42 (d, J=9.0 Hz, 1H), 6.95 (s, 1H), 4.94-4.90 (m, 1H), 4.13 (s, 3H), 4.04-3.90 (m, 2H), 3.71-3.59 (m, 6H), 3.41-3.11 (m, 4H), 2.96 (s, 3H), 2.12-2.05 (m, 2H), 1.91-1.76 (m, 2H).

### Example 54:5-(2-[[5-methoxy-6-(4-methylpiperazin-1-yl)pyridin-3-yl]amino]pyrimidin-4-yl)-2-(oxan-4-yloxy)benzonitrile (54)

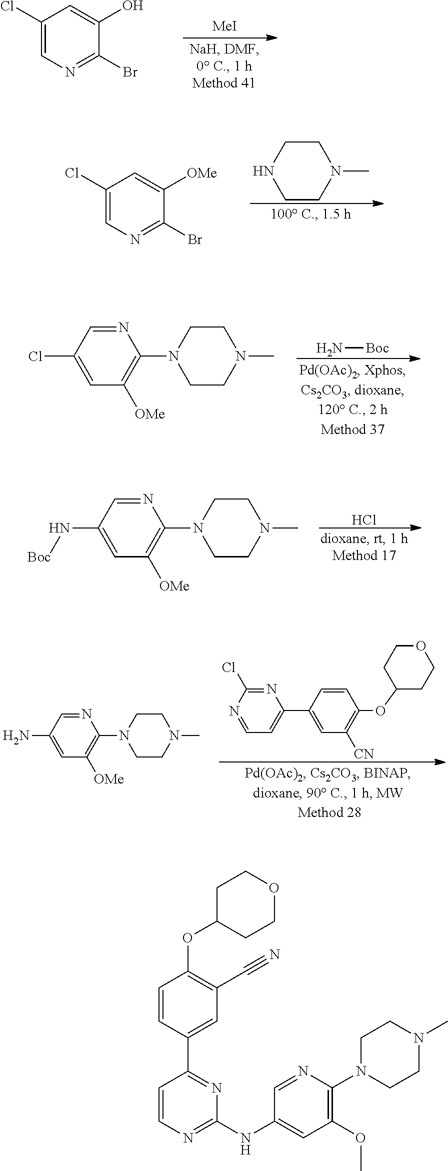

### Method 41

2-bromo-5-chloro-3-methoxypyridine: At 0° C., to a solution of 2-bromo-5-chloropyridin-3-ol (0.95 g, 4.56 mmol) in N,N-dimethylformamide (10 mL) was added sodium hydride (180 mg, 7.50 mmol) in portions. The resulting mixture was stirred for 20 min, and then was added by Mel (741 mg, 5.22 mmol) at 0° C. The resulting mixture was stirred for 0.5 h at 0° C., warmed up to room temperature and stirred for 16 h at room temperature. When the reaction was done, it was quenched by the addition of water (50 mL). The resulting mixture was extracted with ethyl acetate (50 mL×3). The organic phases were combined, washed with brine and dried over Na₂SO₄. The solvent was removed under reduced pressure and the residue was purified by flash chromatography eluting with EtOAc in hexane (0% to 2% gradient) to yield 2-bromo-5-chloro-3-methoxypyridine as a white solid (764 mg, 75%). MS: m/z=221.8 [M+H]⁺.

1-(5-chloro-3-methoxypyridin-2-yl)-4-methylpiperazine: 2-bromo-5-chloro-3-methoxypyridine (382 mg, 1.72 mmol) was dissolved in 1-methylpiperazine (1.65 g, 16.5 mmol) at room temperature. The solution was then stirred for 1.5 h at 100° C. When the reaction was done, it was quenched by sat. NaHCO₃ solution (20 mL). The resulting mixture was extracted with ethyl acetate (30 mL×3). The organic phases were combined, washed with brine and dried over Na₂SO₄. The solvent was removed under reduced pressure to yield 1-(5-chloro-3-methoxypyridin-2-yl)-4-methylpiperazine as a light yellow solid (423 mg, 98%). MS: m/z=241.9 [M+H]⁺.

1-(4-methoxy-6-nitropyridin-3-yl)-4-methylpiperazine: 1-(4-methoxy-6-nitropyridin-3-yl)-4-methylpiperazine was prepared from 5-bromo-4-methoxy-2-nitropyridine and 1-methylpiperazine using Method 28. The product was purified by flash chromatography eluting with MeOH in EtOAc (0% to 20% gradient) to yield 1-(4-methoxy-6-nitropyridin-3-yl)-4-methylpiperazine as an yellow solid (326 mg, 63%). MS: m/z=253.1 [M+H]⁺.

5-(2-[[5-methoxy-6-(4-methylpiperazin-1-yl)pyridin-3-yl]amino]pyrimidin-4-yl)-2-(oxan-4-yloxy)benzonitrile: 5-(2-[[5-methoxy-6-(4-methylpiperazin-1-yl)pyridin-3-yl]amino]pyrimidin-4-yl)-2-(oxan-4-yloxy)benzonitrile was prepared from 1-(5-chloro-3-methoxypyridin-2-yl)-4-methylpiperazine, 5-(2-chloropyrimidin-4-yl)-2-(tetrahydro-2H-pyran-4-yloxy)benzonitrile and tert-butyl carbamate using Method 37, 17, and 28. The final product was purified by prep-HPLC under the following conditions: column, Atlantis Prep T3 OBD Column, 250×19 mm 5 um; mobile phase, acetonitrile in water (with 10 mmol/L NH₄HCO₃), 35% to 60% gradient in 8 min; detector, UV 254 nm. 5-(2-[[5-methoxy-6-(4-methylpiperazin-1-yl)pyridin-3-yl]amino]pyrimidin-4-yl)-2-(oxan-4-yloxy)benzonitrile was obtained as gray solid (29 mg, 14% for 3 steps). HPLC: 96.0% purity, RT=1.52 min. MS: m/z=502.2 [M+H]⁺. ¹H NMR (400 MHz, Methanol-d₄) δ 8.50-8.37 (m, 3H), 8.15 (d, J=2.2 Hz, 1H), 7.93 (d, J=2.3 Hz, 1H), 7.38 (d, J=9.0 Hz, 1H), 7.29 (d, J=5.2 Hz, 1H), 4.95-4.88 (m, 1H), 4.05-3.88 (m, 5H), 3.72-3.61 (m, 2H), 3.23-3.33 (m, 4H), 2.67-2.62 (m, 4H), 2.36 (s, 3H), 2.16-2.06 (m, 2H), 1.93-1.78 (m, 2H).

### Example 55: 2-(2-[[4-methoxy-5-(4-methylpiperazin-1-yl)pyridin-2-yl]amino]pyrimidin-4-yl)-5-(oxan-4-yloxy)pyridine-4-carbonitrile hydrochloride (55)

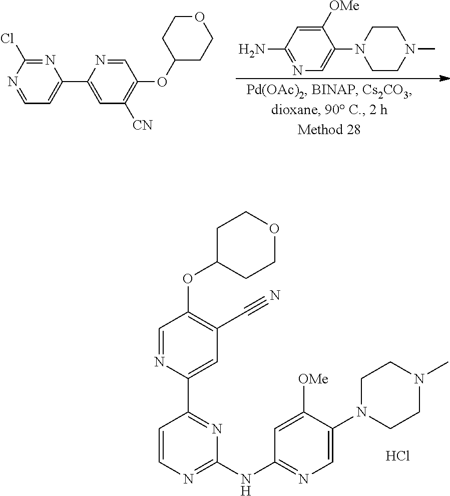 [SMILES: N#Cc1cc(-c2ccnc(Cl)n2)ncc1OC1CCOCC1 ; COc1cc(N)ncc1N1CCN(C)CC1 ; COc1cc(Nc2nccc(-c3cc(C#N)c(OC4CCOCC4)cn3)n2)ncc1N1CCN(C)CC1 ; Cl]

The title compound was prepared from 4-methoxy-5-(4-methylpiperazin-1-yl)pyridin-2-amine and 2-(2-chloropyrimidin-4-yl)-5-(tetrahydro-2H-pyran-4-yloxy)isonicotinonitrile using Method 28. The final product was purified by prep-HPLC under the following conditions: column, XBridge Prep Phenyl OBD Column, 250×19 mm 5 um; mobile phase, acetonitrile in water (with 0.05% HCl), 30% to 60% gradient in 8 min; detector, UV 254 nm. 2-(2-[[4-methoxy-5-(4-methylpiperazin-1-yl)pyridin-2-yl]amino]pyrimidin-4-yl)-5-(oxan-4-yloxy)pyridine-4-carbonitrile hydrochloride was obtained as an yellow solid (18 mg, 14%). HPLC: 96.4% purity, RT=2.11 min. MS: m/z=503.4 [M+H]⁺. ¹H NMR (300 MHz, Methanol-d₄) δ 8.99-8.90 (m, 2H), 8.81 (s, 1H), 8.26-8.17 (m, 1H), 8.03 (s, 1H), 7.14 (s, 1H), 5.28-5.21 (m, 1H), 4.29 (s, 3H), 4.19-4.05 (m, 2H), 3.87-3.71 (m, 6H), 3.54-3.44 (m, 2H), 3.40-3.25 (m, 2H), 3.11 (s, 3H), 2.35-2.24 (m, 2H), 2.11-1.94 (m, 2H).

### Example 56: 2-(2-[[5-methoxy-6-(4-methylpiperazin-1-yl)pyridin-3-yl]amino]pyrimidin-4-yl)-5-(oxan-4-yloxy)pyridine-4-carbonitrile (56)

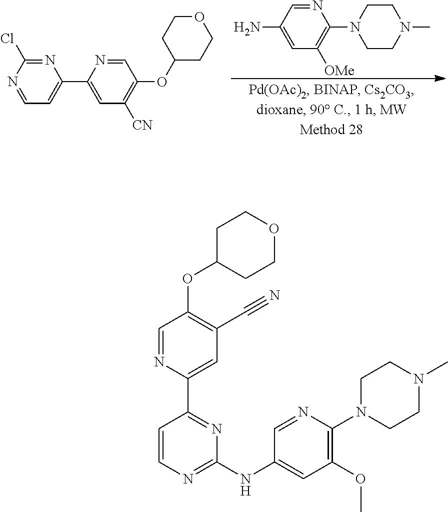

The title compound was prepared from 5-methoxy-6-(4-methylpiperazin-1-yl)pyridin-3-amine and 2-(2-chloropyrimidin-4-yl)-5-(tetrahydro-2H-pyran-4-yloxy)isonicotinonitrile using Method 28. The final product was purified by prep-HPLC under the following conditions: column, XBridge Prep Phenyl OBD Column, 250×19 mm 5 um; mobile phase, acetonitrile in water (with 10 mmol/L NH₄HCO₃), 30% to 60% gradient in 8 min; detector, UV 254 nm. 2-(2-[[5-methoxy-6-(4-methylpiperazin-1-yl)pyridin-3-yl]amino]pyrimidin-4-yl)-5-(oxan-4-yloxy)pyridine-4-carbonitrile was obtained as an yellow solid (49 mg, 36%). HPLC: 98.2% purity, RT=2.36 min. MS: m/z=503.2 [M+H]⁺. ¹H NMR (300 MHz, Methanol-d₄) δ 8.72 (s, 1H), 8.59 (s, 1H), 8.50 (d, J=5.1 Hz, 1H), 8.15-8.08 (m, 1H), 7.94-7.86 (m, 1H), 7.64 (d, J=5.2 Hz, 1H), 5.10-4.98 (m, 1H), 4.04-3.90 (m, 5H), 3.71-3.57 (m, 2H), 3.38-3.29 (m, 4H), 2.64-2.58 (m, 4H), 2.33 (s, 3H), 2.19-2.08 (m, 2H), 1.93-1.76 (m, 2H).

### Example 57: 5-(2-[[5-methoxy-6-(1-methylpiperidin-4-yl)pyridin-3-yl]amino]pyrimidin-4-yl)-2-(oxan-4-yloxy)benzonitrile (57)

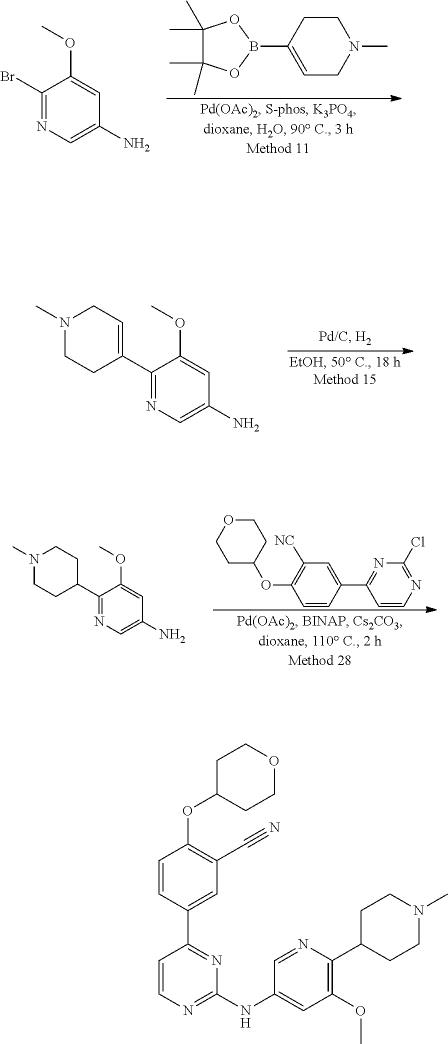

The title compound was prepared from 6-bromo-5-methoxypyridin-3-amine, 1-methyl-4-(4,4,5,5-tetramethyl-1,3,2-dioxaborolan-2-yl)-1,2,3,6-tetrahydropyridine and 5-(2-chloropyrimidin-4-yl)-2-(tetrahydro-2H-pyran-4-yloxy)benzonitrile using Method 11, 15 and 28. The final product was purified by prep-HPLC under the following conditions: column, XBridge Prep C18 Column, 150×19 mm, 5 um; mobile phase, acetonitrile in water (with 0.05% NH₃.H₂O), 48% to 63% gradient in 8 min; detector, UV 254 nm. 5-(2-[[5-methoxy-6-(1-methylpiperidin-4-yl)pyridin-3-yl]amino]pyrimidin-4-yl)-2-(oxan-4-yloxy)benzonitrile was obtained as a light yellow solid (68 mg, 54% for 3 steps). HPLC: 99.4% purity, RT=2.95 min. MS: m/z=501.5 [M+H]⁺. ¹H NMR (400 MHz, DMSO-d₆) δ 9.80 (s, 1H), 8.60-8.53 (m, 2H), 8.47-8.39 (m, 2H), 8.07-8.01 (m, 1H), 7.60-7.47 (m, 2H), 5.00-4.89 (m, 1H), 3.93-3.81 (m, 5H), 3.61-3.50 (m, 2H), 2.98-2.80 (m, 3H), 2.18 (s, 3H), 2.12-1.57 (m, 10H).

### Example 58: 6-([4-[3-cyano-4-(oxan-4-yloxy)phenyl]pyrimidin-2-yl]amino)-5-methoxy-N,N-dimethylpyridine-3-carboxamide (58)

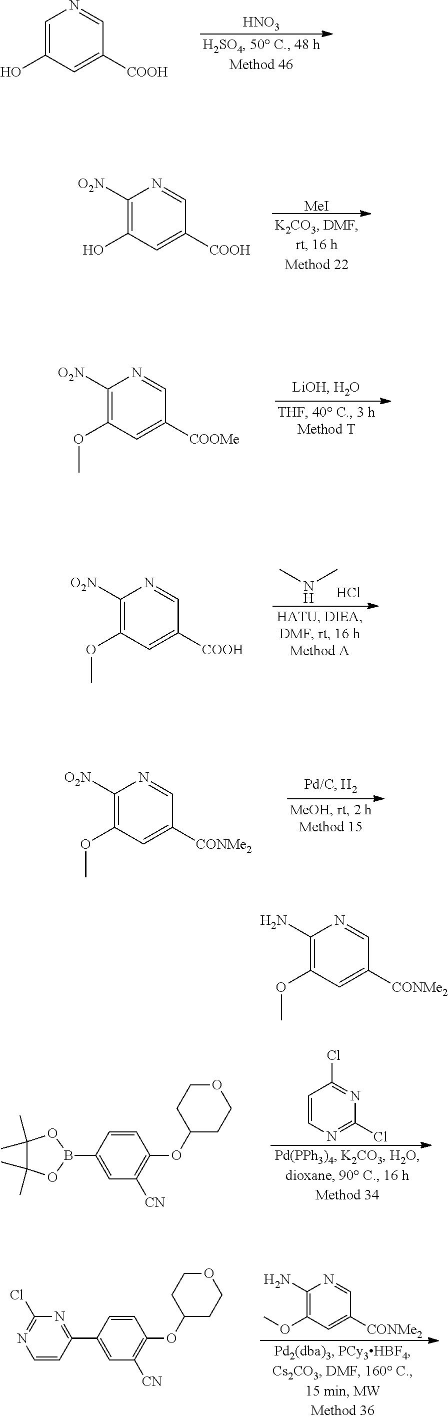

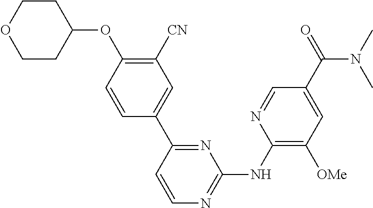 [SMILES: COc1cc(C(=O)N(C)C)cnc1Nc1nccc(-c2ccc(OC3CCOCC3)c(C#N)c2)n1]

### Method 46

5-hydroxy-6-nitropyridine-3-carboxylic acid: At 0° C., to a solution of 5-hydroxypyridine-3-carboxylic acid (6.65 g, 47.80 mmol) in sulfuric acid (9 mL) was added HNO₃ (12.60 g, 0.2 mol) dropwise. The resulting mixture was stirred for 48 h at 55° C. When the reaction was done, the reaction mixture was diluted with ice water (100 mL). The pH value of the mixture was adjusted to 5 with sodium hydroxide solution (5 M). The resulting mixture was extracted with isopropyl alcohol (200 mL×3). The organic phases were combined and concentrated under reduced pressure to yield 5-hydroxy-6-nitropyridine-3-carboxylic acid as an yellow solid (8.00 g, 91%).

### Method 22

5-hydroxy-6-nitropyridine-3-carboxylic acid: At room temperature, to a solution of 5-hydroxy-6-nitropyridine-3-carboxylic acid (4.80 g, 26.07 mmol) in N,N-dimethylformamide (20 mL) was added potassium carbonate (8.5 g, 61.50 mmol), then iodomethane (8.74 g, 61.58 mmol) was added slowly. The resulting mixture was stirred for 16 h at room temperature. When the reaction was done, the reaction mixture was diluted with ice water (60 mL) and extracted with ethyl acetate (100 mL×3). The organic phases were combined, washed with brine and dried over Na₂SO₄. The solvent was removed under reduced pressure and the residue was purified by flash chromatography eluting with EtOAc in petroleum ether (0% to 56% gradient) to yield methyl 5-methoxy-6-nitropyridine-3-carboxylate as an yellow solid (438 mg, 8%). MS: m/z=213.1 [M+H]⁺.

6-([4-[3-cyano-4-(oxan-4-yloxy)phenyl]pyrimidin-2-yl]amino)-5-methoxy-N,N-dimethylpyridine-3-carboxamide: 6-([4-[3-cyano-4-(oxan-4-yloxy)phenyl]pyrimidin-2-yl]amino)-5-methoxy-N,N-dimethylpyridine-3-carboxamide was prepared from methyl 5-methoxy-6-nitronicotinate, dimethylamine hydrochloride, 2-(tetrahydro-2H-pyran-4-yloxy)-5-(4,4,5,5-tetramethyl-1,3,2-dioxaborolan-2-yl)benzonitrile, and 2,4-dichloropyrimidine using Method T, A, 15, 34 and 36. The final product was purified by prep-HPLC under the following conditions: column, XBridge Prep C18 Column, 150×19 mm, 5 um; mobile phase, acetonitrile in water (with 10 mmol/L NR₄HCO₃), 20% to 50% gradient in 7 min; detector, UV 254 nm. 6-([4-[3-cyano-4-(oxan-4-yloxy)phenyl]pyrimidin-2-yl]amino)-5-methoxy-N,N-dimethylpyridine-3-carboxamide was obtained as white solid (24 mg, 3.8% for 5 steps). HPLC: 99.0% purity, RT=1.22 min. MS: m/z=475.2 [M+H]⁺. ¹H NMR (400 MHz, DMSO-d₆) δ 9.28 (s, 1H), 8.58-8.32 (m, 3H), 8.04-7.99 (m, 1H), 7.55-7.46 (m, 3H), 4.95-4.90 (m, 1H), 3.99-3.78 (m, 5H), 3.60-3.50 (m, 2H), 3.03 (s, 6H), 2.07-1.98 (m, 2H), 1.71-1.66 (m, 2H).

### Example 59: 5-(2-[[5-methoxy-6-(1-methylpiperidin-4-yl)pyridin-3-yl]amino]pyrimidin-4-yl)-2-(oxan-4-yloxy)benzonitrile (59)

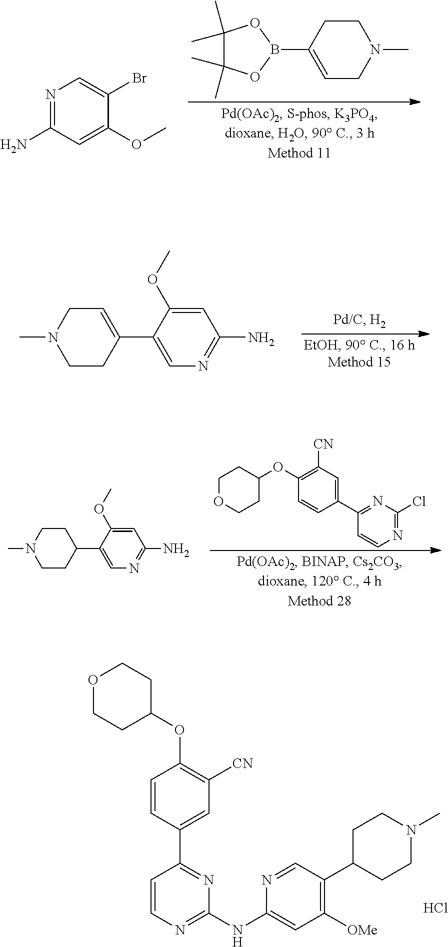

The title compound was prepared from 5-bromo-4-methoxypyridin-2-amine, 1-methyl-4-(4,4,5,5-tetramethyl-1,3,2-dioxaborolan-2-yl)-1,2,3,6-tetrahydropyridine and 5-(2-chloropyrimidin-4-yl)-2-(tetrahydro-2H-pyran-4-yloxy)benzonitrile using Method 11, 15 and 28. The final product was purified by prep-HPLC under the following conditions: column, XBridge Prep C18 Column, 150×19 mm, 5 um; mobile phase, acetonitrile in water (with 0.05% NH₃.H₂O), 45% to 60% gradient in 8 min; detector, UV 254 nm. 5-(2-[[4-methoxy-5-(1-methylpiperidin-4-yl)pyridin-2-yl]amino]pyrimidin-4-yl)-2-(oxan-4-yloxy)benzonitrile hydrochloride was obtained as a light yellow solid (23 mg, 19% for 3 steps). HPLC: 97.0% purity, RT=3.06 min. MS: m/z=501.2 [M+H]⁺. ¹H NMR (300 MHz, Methanol-d₄) δ 8.84-8.76 (m, 1H), 8.64-8.48 (m, 2H), 8.16 (s, 1H), 7.86-7.77 (m, 1H), 7.53-7.44 (m, 1H), 7.01 (s, 1H), 5.10-4.93 (m, 1H), 4.19 (s, 3H), 4.10-3.96 (m, 2H), 3.80-3.60 (m, 4H), 3.32-3.17 (m, 2H), 2.96 (s, 3H), 2.32-1.75 (m, 8H).

### Example 60: 6-[(4-[3-cyano-4-[(oxan-4-yl)amino]phenyl]pyrimidin-2-yl)amino]-5-methoxy-N,N-dimethylpyridine-3-carboxamide (60)

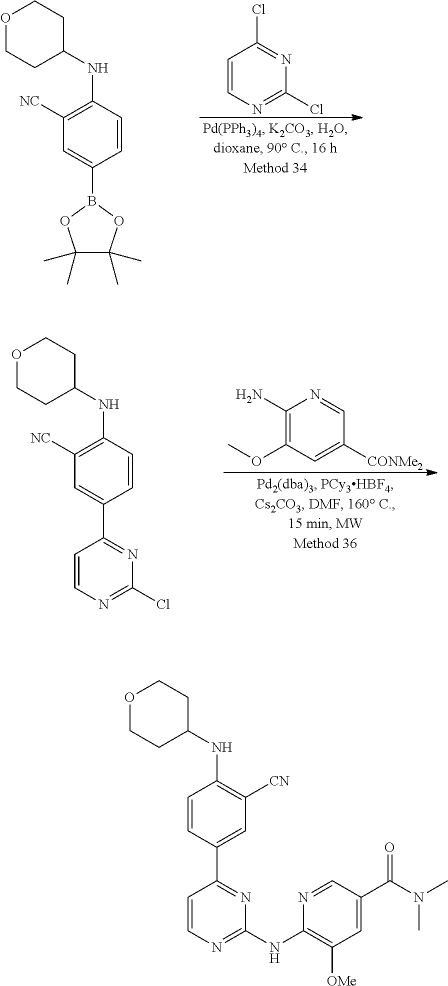

The title compound was prepared from 2-(tetrahydro-2H-pyran-4-ylamino)-5-(4,4,5,5-tetramethyl-1,3,2-dioxaborolan-2-yl)benzonitrile, 2,4-dichloropyrimidine and 6-amino-5-methoxy-N,N-dimethylnicotinamide using Method 34 and 36. The final product was purified by prep-HPLC under the following conditions: column, XBridge Prep C18 Column, 150×19 mm, 5 um; mobile phase, acetonitrile in water (with 10 mmol/L NH₄HCO₃), 20% to 50% gradient in 8 min; detector, UV 254 nm. 6-[(4-[3-cyano-4-[(oxan-4-yl)amino]phenyl]pyrimidin-2-yl)amino]-5-methoxy-N,N-dimethylpyridine-3-carboxamide was obtained as a white solid (24 mg, 3.5% for 2 steps). HPLC: 99.1% purity, RT=1.17 min. MS: m/z=474.3 [M+H]⁺. ¹H NMR (300 MHz, DMSO-d₆) δ 9.11 (s, 1H), 8.46-8.38 (m, 1H), 8.31-8.23 (m, 1H), 8.20-8.10 (m, 1 H), 8.03-7.96 (m, 1H), 7.50-7.35 (m, 2H), 7.02 (d, J=9.2 Hz, 1H), 6.31 (d, J=8.0 Hz, 1H), 3.94-3.70 (m, 6H), 3.50-3.35 (m, 2H), 3.02 (s, 6H), 1.89-1.78 (m, 2H), 1.72-1.54 (m, 2H).

### Example 61: 5-[(4-[3-cyano-4-[(oxan-4-yl)amino]phenyl]pyrimidin-2-yl)amino]-4-methoxy-N,N-dimethylpyridine-2-carboxamide (61)

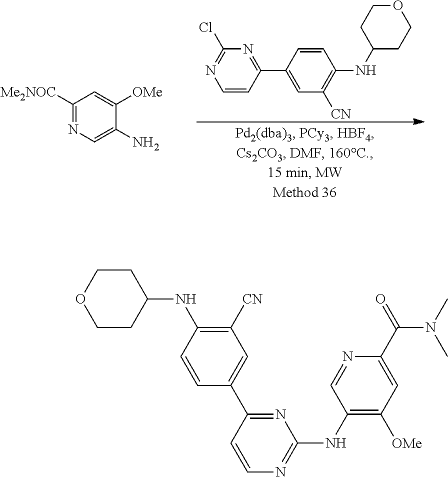

The title compound was prepared from 5-(2-chloropyrimidin-4-yl)-2-(oxan-4-yloxy)benzonitrile and 5-amino-4-methoxy-N,N-dimethylpyridine-2-carboxamide using Method 36. The final product was purified by prep-HPLC under the following conditions: column, XBridge Prep C18 Column, 150×19 mm, 5 um; mobile phase, acetonitrile in water (with 10 mmol/L NH₄HCO₃), 25% to 60% gradient in 8 min; detector, UV 254 nm. 5-[(4-[3-cyano-4-[(oxan-4-yl)amino]phenyl]pyrimidin-2-yl)amino]-4-methoxy-N,N-dimethylpyridine-2-carboxamide was obtained as withe solid (12 mg, 15%). HPLC: 96.8% purity, RT=1.30 min. MS: m/z=474.0 [M+H]⁺. ¹H NMR (300 MHz, Methanol-d₄) δ 9.57 (s, 1H), 8.47-8.39 (m, 1H), 8.29-8.16 (m, 2H), 7.33-7.25 (m, 2H), 7.00 (d, J=9.0 Hz, 1H), 4.05 (s, 3H), 4.01-3.93 (m, 2H), 3.86-3.72 (m, 1H), 3.63-3.48 (m, 2H), 3.11 (s, 3H), 307 (s, 3H), 2.05-1.94 (m, 2H), 1.75-1.55 (m, 2H).

### Example 62: 5-[(4-[3-cyano-4-[(oxan-4-yl)amino]phenyl]pyrimidin-2-yl)amino]-6-methoxy-N,N-dimethylpyridine-2-carboxamide (62)

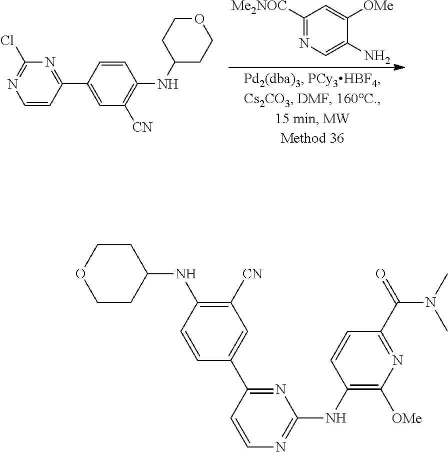

The title compound was prepared from 5-(2-chloropyrimidin-4-yl)-2-[(oxan-4-yl)amino]benzonitrile and 5-amino-6-methoxy-N,N-dimethylpyridine-2-carboxamide using Method 36. The final product was purified by prep-HPLC under the following conditions: column, XBridge Prep C₁₋₈ Column, 150×19 mm, 5 um; mobile phase, acetonitrile in water (with 10 mmol/L NR₄HCO₃), 20% to 50% gradient in 8 min; detector, UV 254 nm. 5-[(4-[3-cyano-4-[(oxan-4-yl)amino]phenyl]pyrimidin-2-yl)amino]-6-methoxy-N,N-dimethylpyridine-2-carboxamide was obtained as a light yellow solid (25 mg, 11%). HPLC: 98.2% purity, RT=1.57 min. MS: m/z=474.2 [M+H]⁺. ¹H NMR (300 MHz, DMSO-d₆) δ 8.63 (d, J=8.0 Hz, 1H), 8.50 (d, J=5.4 Hz, 1H), 8.39-8.31 (m, 2H), 8.28-8.18 (m, 1H), 7.46 (d, J=5.4 Hz, 1H), 7.31 (d, J=8.0 Hz, 1H), 7.05 (d, J=9.1 Hz, 1H), 6.38 (d, J=8.1 Hz, 1H), 3.98 (s, 3H), 3.90-3.66 (m, 3H), 3.50-3.36 (m, 2H), 3.12 (s, 3H), 3.00 (s, 3H), 1.90-1.78 (m, 2H), 1.73-1.59 (m, 2H).

### Example 63: 6-[(4-[3-cyano-4-[(oxan-4-yl)amino]phenyl]pyrimidin-2-yl)amino]-2-methoxy-N,N-dimethylpyridine-3-carboxamide (63)

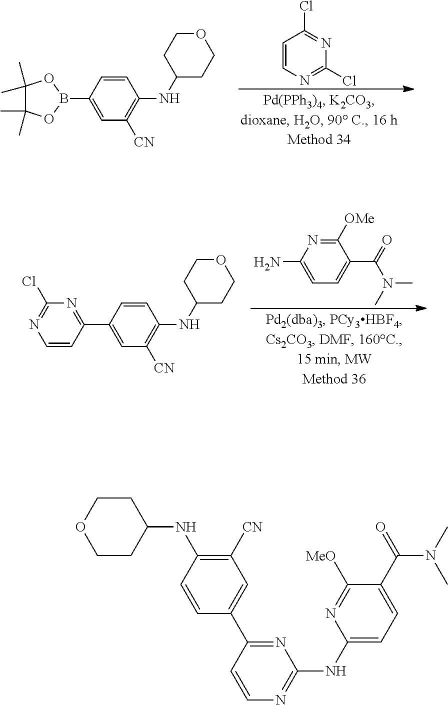 [SMILES: CC1(C)OB(c2ccc(NC3CCOCC3)c(C#N)c2)OC1(C)C ; Clc1ccnc(Cl)n1 ; N#Cc1cc(-c2ccnc(Cl)n2)ccc1NC1CCOCC1 ; COc1nc(N)ccc1C(=O)N(C)C ; COc1nc(Nc2nccc(-c3ccc(NC4CCOCC4)c(C#N)c3)n2)ccc1C(=O)N(C)C]

The title compound was prepared from 2-(tetrahydro-2H-pyran-4-ylamino)-5-(4,4,5,5-tetramethyl-1,3,2-dioxaborolan-2-yl)benzonitrile, 2,4-dichloropyrimidine, and 6-amino-2-methoxy-N,N-dimethylnicotinamide using Method 34 and 36. The final product was purified by prep-HPLC under the following conditions: column, XBridge Prep C18 OBD Column, 150×19 mm, 5 um; mobile phase, acetonitrile in water (with 0.05% NH₃.H₂O), 40% to 70% gradient in 8 min; detector, UV 254 nm. 6-[(4-[3-cyano-4-[(oxan-4-yl)amino]phenyl]pyrimidin-2-yl)amino]-2-methoxy-N,N-dimethylpyridine-3-carboxamide was obtained as a white solid (28 mg, 15% for 2 steps). HPLC: 92.4% purity, RT=2.67 min. MS: m/z=474.2 [M+H]⁺. ¹H NMR (300 MHz, DMSO-de) δ 9.75 (s, 1H), 8.59-8.50 (m, 1H), 8.44-8.36 (m, 1H), 8.32-8.17 (m, 1H), 7.97-7.88 (m, 1H), 7.68-7.59 (m, 1H), 7.54-7.46 (m, 1H), 7.12-6.94 (m, 1H), 6.46-6.36 (m, 1H), 3.95-3.85 (m, 5H), 3.80-3.74 (m, 1H), 3.52-3.37 (m, 2H), 2.97 (s, 3H), 2.83 (s, 3H), 1.91-1.79 (m, 2H), 1.75-1.57 (m, 2H).

### Example 64: 6-[(4-[6-cyano-5-[(oxan-4-yl)amino]pyridin-2-yl]pyrimidin-2-yl)amino]-2-methoxy-N,N-dimethylpyridine-3-carboxamide (64)

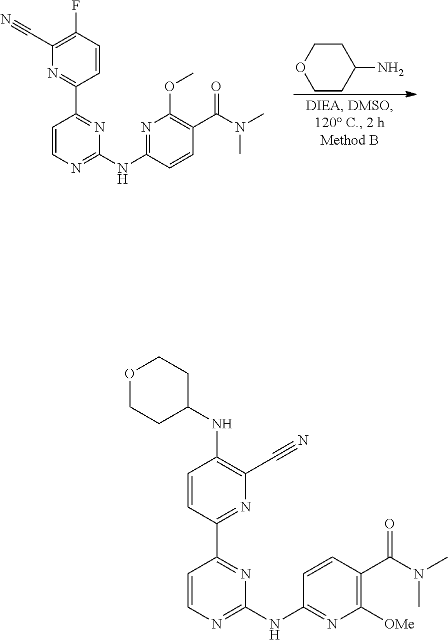 [SMILES: COc1nc(Nc2nccc(-c3ccc(F)c(C#N)n3)n2)ccc1C(=O)N(C)C ; NC1CCOCC1 ; COc1nc(Nc2nccc(-c3ccc(NC4CCOCC4)c(C#N)n3)n2)ccc1C(=O)N(C)C]

The title compound was prepared from oxan-4-amine and 6-[[4-(6-cyano-5-fluoropyridin-2-yl)pyrimidin-2-yl]amino]-2-methoxy-N,N-dimethylpyridine-3-carboxamide using Method B. The final product was purified by prep-HPLC under the following conditions: column, XBridge Prep OBD C18 Column, 150×19 mm, 5 um mobile phase, acetonitrile in water (with 0.05% NH₃.H₂O), 40% to 42% gradient in 8 min; detector, UV 254 nm. 6-[(4-[6-cyano-5-[(oxan-4-yl)amino]pyridin-2-yl]pyrimidin-2-yl)amino]-2-methoxy-N,N-dimethylpyridine-3-carboxamide was obtained as a white solid (15 mg, 39%). HPLC: 99.5% purity, RT=1.32 min. MS: m/z=475.0 [M+H]⁺. ¹H NMR (300 MHz, DMSO-d₆) δ 9.83 (s, 1H), 8.66-8.57 (m, 1H), 8.44-8.34 (m, 1H), 7.96-7.87 (m, 1H), 7.68-7.56 (m, 3H), 6.79-6.70 (m, 1H), 3.99-3.67 (m, 6H), 3.48-3.34 (m, 2H), 2.95 (s, 3H), 2.82 (s, 3H), 1.92-1.51 (m, 4H).

### Example 65: 5-(2-[[5-(4-methylpiperazin-1-yl)pyridin-2-yl]amino]pyrimidin-4-yl)-2-(oxan-4-yloxy)benzonitrile (65)

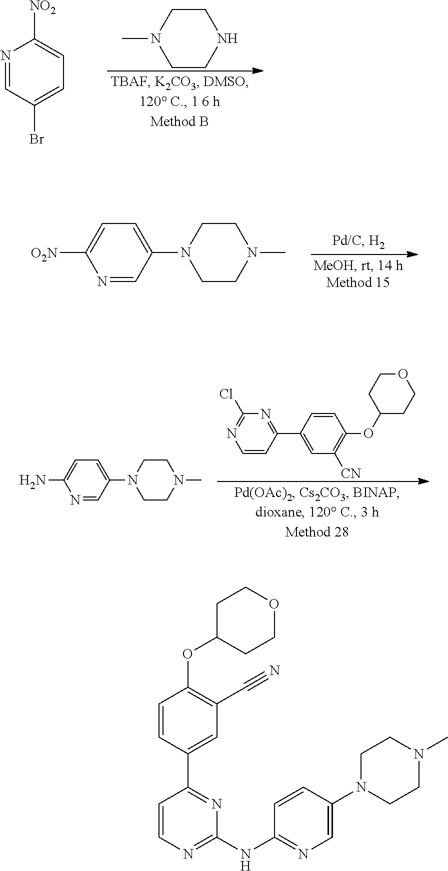

The title compound was prepared from 5-bromo-2-nitropyridine, 1-methyl-4-(6-nitropyridin-3-yl)piperazine, and 5-(2-chloropyrimidin-4-yl)-2-(tetrahydro-2H-pyran-4-yloxy)benzonitrile using Method B, 15, and 28. The final product was purified by prep-HPLC under the following conditions: column, XBridge Shield RP18 OBD Column, 150×19 mm, 5 um; mobile phase, acetonitrile in water (with 0.05% NH₃.H₂O), 31% to 53% gradient in 8 min; detector, UV 254 nm. 5-(2-[[5-(4-methylpiperazin-1-yl)pyridin-2-yl]amino]pyrimidin-4-yl)-2-(oxan-4-yloxy)benzonitrile was obtained as a yellow solid (25 mg, 1% for 5 steps). HPLC: 99.7% purity, RT=1.17 min. MS: m/z=472.2 [M+H]⁺. ¹H NMR (300 MHz, DMSO-d₆) δ 9.58 (s, 1H), 8.59-8.51 (m, 2H), 8.51-8.41 (m, 1H), 8.15-8.05 (m, 1H), 8.05-7.97 (m, 1H), 7.59-7.39 (m, 3H), 4.99-4.88 (m, 1H), 3.94-3.81 (m, 2H), 3.63-3.49 (m, 2H), 3.18-3.08 (m, 4H), 2.49-2.43 (m, 4H), 2.23 (s, 3H), 2.10-1.99 (m, 2H), 1.76-1.63 (m, 2H).

### Example 66: 5-{2-[6-(4-Methyl-piperazin-1-yl)-pyridin-3-ylamino]-pyrimidin-4-yl}-2-(tetrahydro-pyran-4-yloxy)-benzonitrile (66)

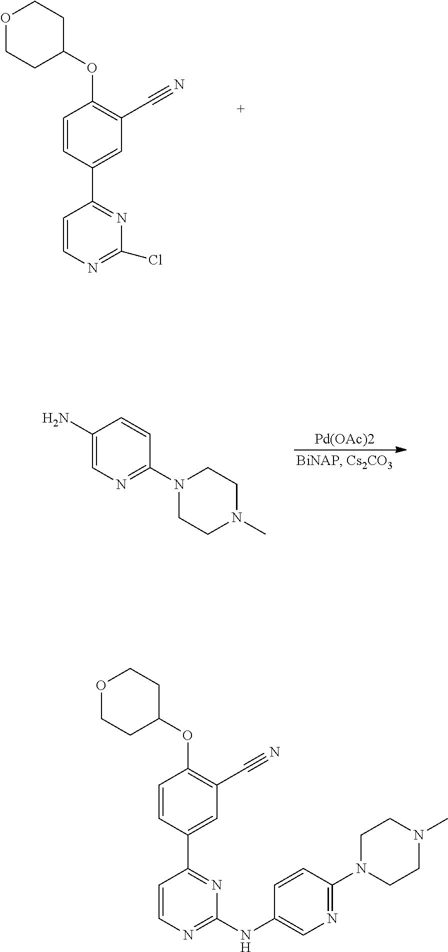 [SMILES: N#Cc1cc(-c2ccnc(Cl)n2)ccc1OC1CCOCC1 ; CN1CCN(c2ccc(N)cn2)CC1 ; CN1CCN(c2ccc(Nc3nccc(-c4ccc(OC5CCOCC5)c(C#N)c4)n3)cn2)CC1 ; "Pd(OAc)2"]

The title compound was prepared from 5-(2-Chloro-pyrimidin-4-yl)-2-(tetrahydro-pyran-4-yloxy)-benzonitrile and 6-(4-Methyl-piperazin-1-yl)-pyridin-3-ylamine using Method 28. The final product was purified by prep-HPLC under the following conditions: column, XBridge Shield RP18 OBD Column, 150×19 mm, 5 um; mobile phase, acetonitrile in water (with 0.05% NH₃.H₂O), 31% to 53% gradient in 8 min; detector, UV 254 nm. 5-(2-[[5-(4-methylpiperazin-1-yl)pyridin-2-yl]amino]pyrimidin-4-yl)-2-(oxan-4-yloxy)benzonitrile was obtained as a white solid. MS: m/z=472.8 [M+H]⁺.

### Example 67: 5-{5-Fluoro-2-[6-methoxy-5-(4-methyl-piperazin-1-yl)-pyridin-2-ylamino]-pyrimidin-4-yl}-2-(tetrahydro-pyran-4-yloxy)-benzonitrile (67)

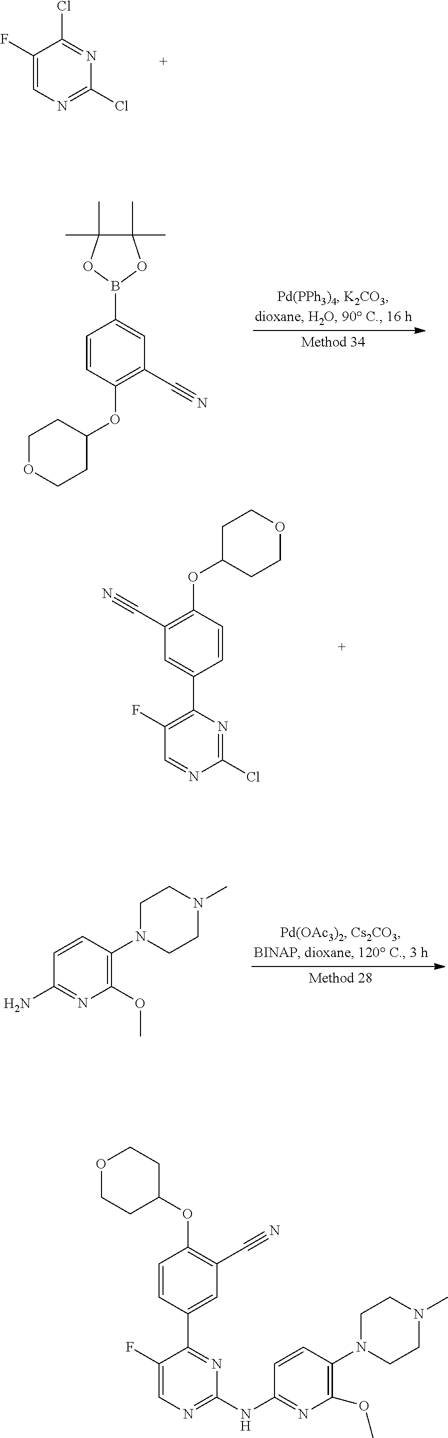 [SMILES: Fc1cnc(Cl)nc1Cl ; CC1(C)OB(c2ccc(OC3CCOCC3)c(C#N)c2)OC1(C)C ; N#Cc1cc(-c2nc(Cl)ncc2F)ccc1OC1CCOCC1 ; COc1nc(N)ccc1N1CCN(C)CC1 ; COc1nc(Nc2ncc(F)c(-c3ccc(OC4CCOCC4)c(C#N)c3)n2)ccc1N1CCN(C)CC1]

The title compound was prepared from 2,4-Dichloro-5-fluoro-pyrimidine (2.60 g; 15.57 mmol; 1.00 eq.), 2-(oxan-4-yloxy)-5-(tetramethyl-1,3,2-dioxaborolan-2-yl)benzonitrile (5.13 g; 15.57 mmol; 1.00 eq.), and, 6-Methoxy-5-(4-methyl-piperazin-1-yl)-pyridin-2-ylamine using Methods 34 and 28 as a white solid (25 mg, 10% for 2 steps). MS: m/z=520.5 [M+H]⁺.

### Example 68: 6-([4-[6-cyano-5-(oxolan-3-yloxy)pyridin-2-yl]pyrimidin-2-yl]amino)-2-methoxy-N,N-dimethylpyridine-3-carboxamide 68

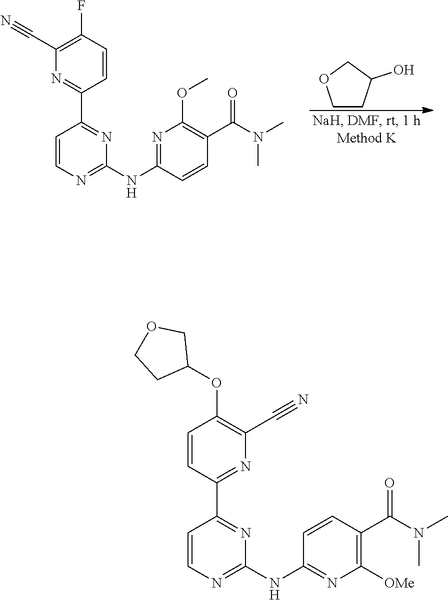 [SMILES: COc1nc(Nc2nccc(-c3ccc(F)c(C#N)n3)n2)ccc1C(=O)N(C)C ; OC1CCOC1 ; COc1nc(Nc2nccc(-c3ccc(OC4CCOC4)c(C#N)n3)n2)ccc1C(=O)N(C)C]

The title compound was prepared from oxolan-3-ol and 6-[[4-(6-cyano-5-fluoropyridin-2-yl)pyrimidin-2-yl]amino]-2-methoxy-N,N-dimethylpyridine-3-carboxamide using Method K. The final product was purified by prep-HPLC under the following conditions: column, XBridge Prep OBD C18 Column, 150×19 mm, 5 um; mobile phase, acetonitrile in water (with 0.05% NH₃.H₂O), 37% to 39% gradient in 8 min; detector, UV 254 nm. 6-([4-[6-cyano-5-(oxolan-3-yloxy)pyridin-2-yl]pyrimidin-2-yl]amino)-2-methoxy-N,N-dimethylpyridine-3-carboxamide was obtained as a white solid (19 mg, 31%). HPLC: 99.0% purity, RT=1.18 min. MS: m/z=462.1 [M+H]⁺. ¹H NMR (300 MHz, DMSO-d₆) δ 9.99 (s, 1H), 8.77-8.61 (m, 2H), 8.10-8.00 (m, 1H), 7.98-7.88 (m, 1H), 7.76-7.62 (m, 2H), 5.42-5.34 (m, 1H), 4.04-3.75 (m, 7H), 2.97 (s, 3H), 2.84 (s, 3H), 2.46-1.99 (m, 2H).

### Example 69: 6-[(4-[3-cyano-4-[(1-methylazetidin-3-yl)oxy]phenyl]pyrimidin-2-yl)amino]-2-methoxy-N,N-dimethylpyridine-3-carboxamide 69

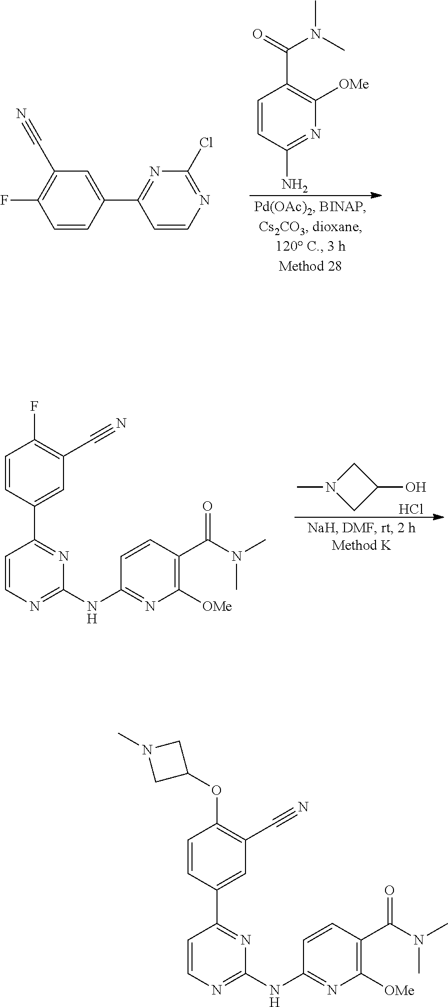

The title compound was prepared from 5-(2-chloropyrimidin-4-yl)-2-fluorobenzonitrile, 6-amino-2-methoxy-N,N-dimethylnicotinamide and 1-methylazetidin-3-ol hydrochloride using Method 28 and K. The final product was purified by prep-HPLC under the following conditions: column, XBridge Prep OBD C18 Column, 150×19 mm, 5 um; mobile phase, acetonitrile in water (with 0.05% NH₃.H₂O), 35% to 65% gradient in 8 min; detector, UV 254 nm. 6-[(4-[3-cyano-4-[(1-methylazetidin-3-yl)oxy]phenyl]pyrimidin-2-yl)amino]-2-methoxy-N,N-dimethylpyridine-3-carboxamide was obtained as withe solid (11 mg, 11% for 2 steps). HPLC: 98.5% purity, RT=2.37 min. MS: m/z=460.3 [M+H]⁺. ¹H NMR (300 MHz, DMSO-d₆) δ 9.87 (s, 1H), 8.69-8.58 (m, 2H), 8.53-8.42 (m, 1H), 7.95-7.86 (m, 1H), 7.69-7.57 (m, 2H), 7.20 (d, J=9.0 Hz, 1H), 5.11-4.97 (m, 1H), 3.92 (s, 3H), 3.86-3.75 (m, 2H), 3.16-3.05 (m, 2H), 2.97 (s, 3H), 2.83 (s, 3H), 2.33 (s, 3H).

### Example 70: 6-[(4-[3-cyano-4-[(1-methylpyrrolidin-3-yl)oxy]phenyl]pyrimidin-2-yl)amino]-2-methoxy-N,N-dimethylpyridine-3-carboxamide hydrochloride 70

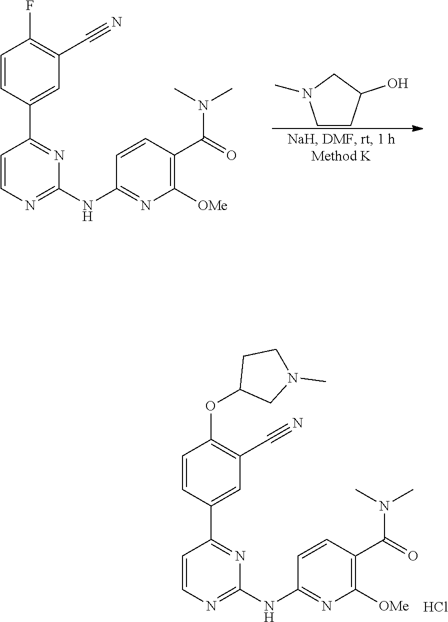

The title compound was prepared from 6-[[4-(3-cyano-4-fluorophenyl)pyrimidin-2-yl]amino]-2-methoxy-N,N-dimethylpyridine-3-carboxamide and 1-methylpyrrolidin-3-ol using Method K. The final product was purified by prep-HPLC under the following conditions: column, XBridge Prep OBD C18 Column, 150×19 mm, 5 um; mobile phase, acetonitrile in water (with 0.05% HCl), 35% to 65% gradient in 8 min; detector, UV 254 nm. 6-[(4-[3-cyano-4-[(1-methylpyrrolidin-3-yl)oxy]phenyl]pyrimidin-2-yl)amino]-2-methoxy-N,N-dimethylpyridine-3-carboxamide hydrochloride was obtained as an yellow solid (25 mg, 28%). HPLC: 98.2% purity, RT=1.24 min. MS: m/z=488.4 [M+H]⁺. ¹H NMR (300 MHz, Methanol-d₄) δ 8.77-8.56 (m, 3H), 7.88-7.74 (m, 2H), 7.63-7.48 (m, 1H), 7.27-7.15 (m, 1H), 5.20-4.92 (m, 1H), 4.12 (s, 3H), 4.06-3.80 (m, 2H), 3.60-3.51 (m, 1H), 3.38-3.29 (m, 1H), 3.09 (s, 3H), 2.93 (s, 6H), 2.55-1.90 (m, 5H).

### Example 71: 6-[(4-[3-cyano-4-[(1-methylpiperidin-4-yl)oxy]phenyl]pyrimidin-2-yl)amino]-2-methoxy-N,N-dimethylpyridine-3-carboxamide hydrochloride 71

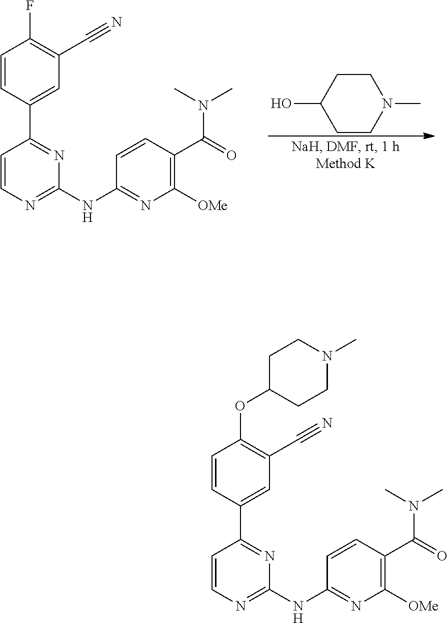

The title compound was prepared from 6-[[4-(3-cyano-4-fluorophenyl)pyrimidin-2-yl]amino]-2-methoxy-N,N-dimethylpyridine-3-carboxamide and 1-methylpiperidin-4-ol using Method K. The final product was purified by prep-HPLC under the following conditions: column, XBridge Prep OBD C18 Column, 150×19 mm, 5 um; mobile phase, acetonitrile in water (with 0.05% HCl), 5% to 50% gradient in 8 min; detector, UV 254 nm. 6-[(4-[3-cyano-4-[(1-methylpiperidin-4-yl)oxy]phenyl]pyrimidin-2-yl)amino]-2-methoxy-N,N-dimethylpyridine-3-carboxamide hydrochloride was obtained as an yellow solid (25 mg, 26%). HPLC: 96.2% purity, RT=2.04 min. MS: m/z=515.2 [M+H]⁺. ¹H NMR (300 MHz, Methanol-d₄) δ 8.52-8.35 (m, 3H), 7.86-7.78 (m, 2H), 7.60-7.51 (m, 1H), 7.24-7.18 (m, 1H), 5.16-4.9 (m, 1H), 4.12 (s, 3H), 3.68-3.64 (m, 2H), 3.54-3.49 (m, 1H), 3.35-3.20 (m, 1H), 3.08 (s, 3H), 2.94 (s, 6H), 2.50-2.02 (m, 4H).

### Example 72: 6-([4-[3-cyano-4-(piperidin-4-yloxy)phenyl]pyrimidin-2-yl]amino)-2-methoxy-N,N-dimethylpyridine-3-carboxamide 72

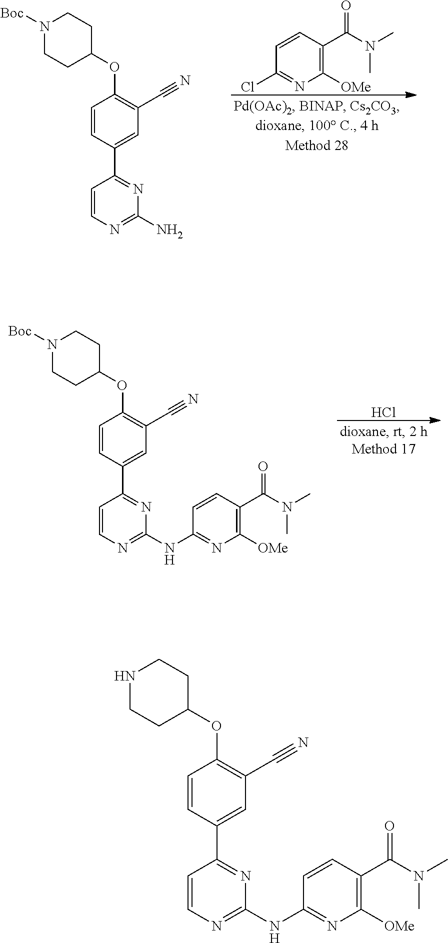 [SMILES: CC(C)(C)OC(=O)N1CCC(Oc2ccc(-c3ccnc(N)n3)cc2C#N)CC1 ; COc1nc(Cl)ccc1C(=O)N(C)C ; COc1nc(Nc2nccc(-c3ccc(OC4CCN(C(=O)OC(C)(C)C)CC4)c(C#N)c3)n2)ccc1C(=O)N(C)C ; Cl ; COc1nc(Nc2nccc(-c3ccc(OC4CCNCC4)c(C#N)c3)n2)ccc1C(=O)N(C)C]

The title compound was prepared from tert-butyl 4-(4-(2-aminopyrimidin-4-yl)-2-cyanophenoxy)piperidine-1-carboxy late and 6-chloro-2-methoxy-N,N-dimethylnicotinamide using Method 28 and 17. The final product was purified by prep-HPLC under the following conditions: column, XBridge Prep C18 Column, 150×19 mm, 5 um; mobile phase, acetonitrile in water (with 0.05% NH₃.H₂O), 32% to 33% gradient in 7 min; detector, UV 254 nm. 6-([4-[3-cyano-4-(piperidin-4-yloxy)phenyl]pyrimidin-2-yl]amino)-2-methoxy-N,N-dimethylpyridine-3-carboxamide was obtained as a white solid (20 mg, 11% for 2 steps). HPLC: 99.1% purity, RT=1.51 min. MS: m/z=474.3 [M+H]⁺. ¹H NMR (300 MHz, DMSO-d₆) δ 9.90 (s, 1H), 8.69-8.56 (m, 2H), 8.53-8.43 (m, 1H), 7.96-7.87 (m, 1H), 7.70-7.58 (m, 2H), 7.56-7.46 (m, 1H), 4.86-4.73 (m, 1H), 3.92 (s, 3H), 3.05-2.91 (m, 5H), 2.83 (s, 3H), 2.68-2.55 (m, 2H), 2.01-1.90 (m, 2H), 1.64-1.49 (m, 2H).

### Example 73: 6-[[4-(3-cyano-4-[[1-(2-hydroxyacetyl)piperidin-4-yl]oxy]phenyl)pyrimidin-2-yl]amino]-2-methoxy-N,N-dimethylpyridine-3-carboxamide 73

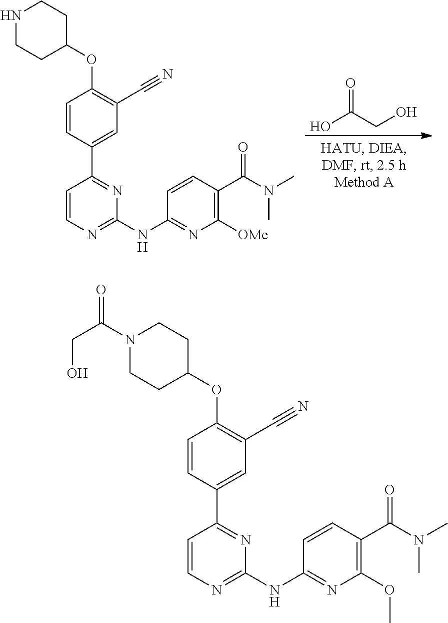 [SMILES: COc1nc(Nc2nccc(-c3ccc(OC4CCNCC4)c(C#N)c3)n2)ccc1C(=O)N(C)C ; O=C(O)CO ; COc1nc(Nc2nccc(-c3ccc(OC4CCN(C(=O)CO)CC4)c(C#N)c3)n2)ccc1C(=O)N(C)C]

The title compound was prepared from 2-hydroxyacetic acid and 6-([4-[3-cyano-4-(piperidin-4-yloxy)phenyl]pyrimidin-2-yl]amino)-2-methoxy-N,N-dimethylpyridine-3-carboxamide using Method A. The final product was purified by prep-HPLC under the following conditions: column, XBridge Prep C18 Column, 150×19 mm, 5 um; mobile phase, acetonitrile in water (with 0.05% NH₃.H₂O), 32% to 37% gradient in 7 min; detector, UV 254 nm. 6-[[4-(3-cyano-4-[[1-(2-hydroxyacetyl)piperidin-4-yl]oxy]phenyl)pyrimidin-2-yl]amino]-2-methoxy-N,N-dimethylpyridine-3-carboxamide was obtained as a white solid (25 mg, 29%). HPLC: 98.9% purity, RT=2.81 min. MS: m/z=532.2 [M+H]⁺. ¹H NMR (300 MHz, DMSO-d₆) δ 9.90 (s, 1H), 8.70-8.58 (m, 2H), 8.56-8.46 (m, 1H), 7.96-7.87 (m, 1H), 7.70-7.52 (m, 3H), 5.05-4.99 (m, 1H), 4.63-4.53 (m, 1H), 4.18-4.09 (m, 2H), 3.92 (s, 3H), 3.82-3.35 (m, 4H), 2.97 (s, 3H), 2.83 (s, 3H), 2.03-1.96 (m, 2H), 1.74-1.68 (m, 2H).

### Example 74: 6-[[4-(3-cyano-4-[[(3R,4S)-3-fluoropiperidin-4-yl]oxy]phenyl)pyrimidin-2-yl]amino]-2-methoxy-N,N-dimethylpyridine-3-cart)oxamide 74

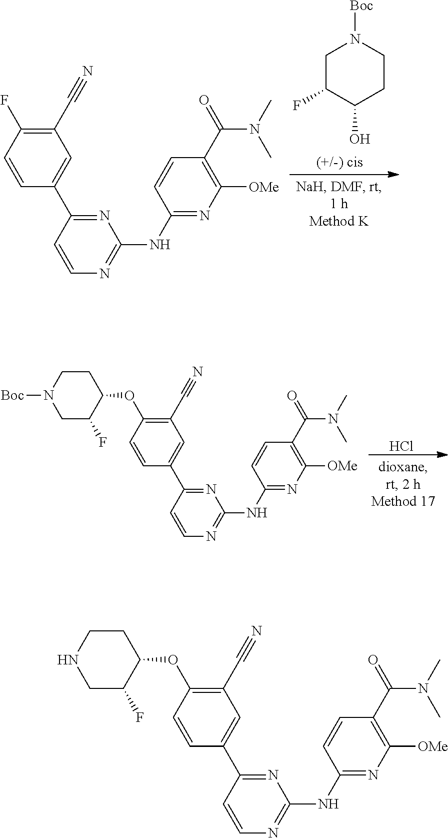

The title compound was prepared from 6-(4-(3-cyano-4-fluorophenyl)pyrimidin-2-ylamino)-2-methoxy-N,N-dimethylnicotinamide and (cis+/−)-tert-butyl 3-fluoro-4-hydroxypiperidine-1-carboxylate using Method K and 17. The final product was purified by prep-HPLC under the following conditions: column, XBridge Prep C18 Column, 150×19 mm, 5 um; mobile phase, acetonitrile in water (with 0.05% NH₃.H₂O), 34% to 35% gradient in 7 min; detector, UV 254 nm. 6-[[4-(3-cyano-4-[[(3R,4S)-3-fluoropiperidin-4-yl]oxy]phenyl)pyrimidin-2-yl]amino]-2-methoxy-N,N-dimethylpyridine-3-carboxamide was obtained as a white solid (18 mg, 41% for 2 steps). HPLC: 99.6% purity, RT=1.26 min. MS: m/z=492.2 [M+H]⁺. ¹H NMR (400 MHz, DMSO-d₆) δ 9.88 (s, 1H), 8.70-8.42 (m, 3H), 7.95-7.84 (m, 1H), 7.69-7.50 (m, 3H), 5.12-4.95 (m, 1H), 4.92-4.70 (m, 1H), 3.90 (s, 3H), 3.17-3.05 (m, 1H), 2.95 (s, 3H), 2.81-2.76 (m, 5H), 2.68-2.54 (m, 1H), 1.94-1.72 (m, 2H).

### Example 75: 6-[[4-(3-cyano-4-[[(3R,4S)-3-fluoro-1-(2-hydroxyacetyl)piperidin-4-yl]oxy]phenyl)pyrimidin-2-yl]amino]-2-methoxy-N,N-dimethylpyridine-3-carboxamide hydrochloride 75

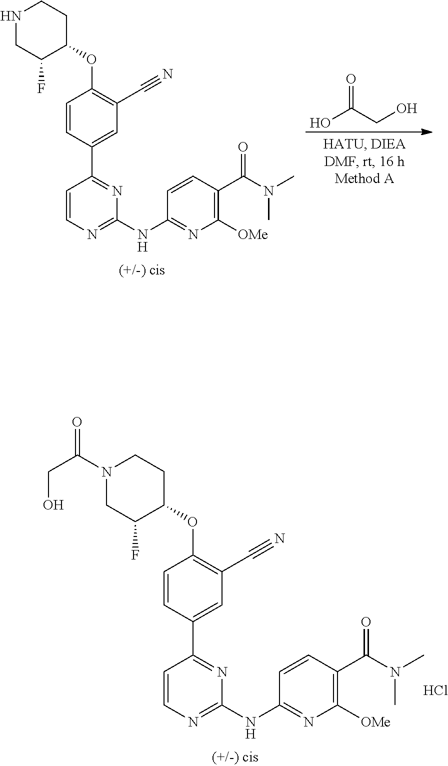 [SMILES: COc1nc(Nc2nccc(-c3ccc(O[C@H]4CCNC[C@H]4F)c(C#N)c3)n2)ccc1C(=O)N(C)C ; O=C(O)CO ; COc1nc(Nc2nccc(-c3ccc(O[C@H]4CCN(C(=O)CO)C[C@H]4F)c(C#N)c3)n2)ccc1C(=O)N(C)C ; Cl]

The title compound was prepared from 6-[[4-(3-cyano-4-[[(3R,4S)-3-fluoropiperidin-4-yl]oxy]phenyl)pyrimidin-2-yl]amino]-2-methoxy-N,N-dimethylpyridine-3-carboxamide and 2-hydroxy acetic acid using Method A. The final product was purified by prep-HPLC under the following conditions: column, XBridge Prep C18 Column, 150×19 mm, 5 um; mobile phase, acetonitrile in water (with 0.05% NH₃.H₂O), 34% to 35% gradient in 7 min; detector, UV 254 nm. 6-[[4-(3-cyano-4-[[(3R,4S)-3-fluoro-1-(2-hydroxyacetyl)piperidin-4-yl]oxy]phenyl)pyrimidin-2-yl]amino]-2-methoxy-N,N-dimethylpyridine-3-carboxamide hydrochloride was obtained as an yellow solid (42 mg, 71%). HPLC: 97.4% purity, RT=2.30 min. MS: m/z=550.2 [M+H]⁺. ¹H NMR (300 MHz, DMSO-d₆) δ 9.41 (br s, 1H), 8.63 (d, J=5.4 Hz, 1H), 8.54 (s, 1H), 8.47-8.40 (m, 1H), 7.84 (d, J=8.1 Hz, 1H), 7.66-7.52 (m, 3H), 5.16-4.86 (m, 2H), 4.20-4.10 (m, 2H), 4.00-3.74 (m, 4H), 3.69-3.61 (m, 2H), 3.37-3.26 (m, 1H), 2.94-2.87 (br s, 6H), 2.02-1.93 (m, 2H).

### Example 76: 6-[(4-[3-cyano-4-[(3,3-difluoropiperidin-4-yl)oxy]phenyl]pyrimidin-2-yl)amino]-2-methoxy-N,N-dimethylpyridine-3-carboxamide 76

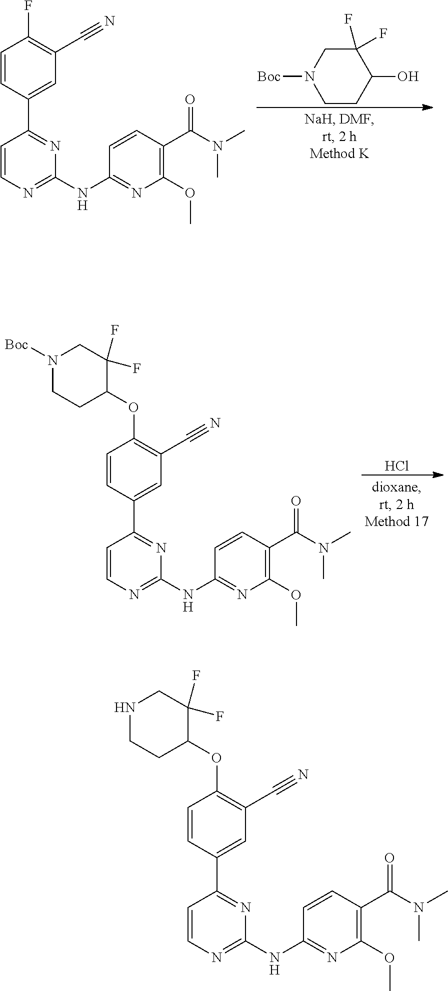 [SMILES: COc1nc(Nc2nccc(-c3ccc(F)c(C#N)c3)n2)ccc1C(=O)N(C)C ; CC(C)(C)OC(=O)N1CCC(O)C(F)(F)C1 ; COc1nc(Nc2nccc(-c3ccc(OC4CCN(C(=O)OC(C)(C)C)CC4(F)F)c(C#N)c3)n2)ccc1C(=O)N(C)C ; Cl ; COc1nc(Nc2nccc(-c3ccc(OC4CCNCC4(F)F)c(C#N)c3)n2)ccc1C(=O)N(C)C]

The title compound was prepared from tert-butyl 3,3-difluoro-4-hydroxy piperidine-1-carboxy late and 6-[[4-(3-cyano-4-fluorophenyl)pyrimidin-2-yl]amino]-2-methoxy-N,N-dimethylpyridine-3-carboxamide using Method K and 17. The final product was purified by prep-HPLC under the following conditions: column, XBridge Prep C18 Column, 150×19 mm, 5 um; mobile phase, acetonitrile in water (with 10 mmol/L NH₄HCO₃ and 0.1% NH₃.H₂O), 35% to 55% gradient in 7 min; detector, UV 254 nm. 6-[(4-[3-cyano-4-[(3,3-difluoropiperidin-4-yl)oxy]phenyl]pyrimidin-2-yl)amino]-2-methoxy-N,N-dimethylpyridine-3-carboxamide was obtained as a white solid (22 mg, 11% for 2 steps). HPLC: 99.4% purity, RT=0.92 min. MS: m/z=510.3 [M+H]⁺. ¹H NMR (400 MHz, DMSO-d₆) δ 9.90 (s, 1H), 8.70-8.60 (m, 2H), 8.56-8.48 (m, 1H), 7.95-7.88 (m, 1H), 7.68-7.61 (m, 3H), 5.29-5.20 (m, 1H), 3.92 (s, 3H), 3.25-2.62 (m, 10H), 2.16-1.77 (m, 2H).

### Example 77: 6-([4-[3-cyano-4-([1-[(1,3-oxazol-4-yl)carbonyl]piperidin-4-yl]oxy)phenyl]pyrimidin-2-yl]amino)-2-methoxy-N,N-dimethylpyridine-3-carboxamide 77

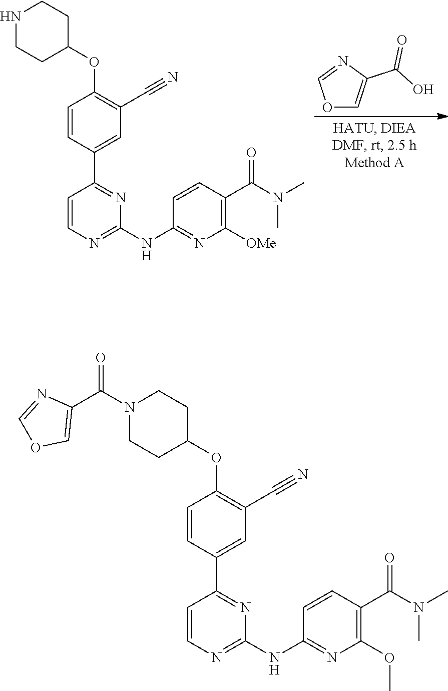 [SMILES: COc1nc(Nc2nccc(-c3ccc(OC4CCNCC4)c(C#N)c3)n2)ccc1C(=O)N(C)C ; O=C(O)c1cocn1 ; COc1nc(Nc2nccc(-c3ccc(OC4CCN(C(=O)c5cocn5)CC4)c(C#N)c3)n2)ccc1C(=O)N(C)C]

The title compound was prepared from 1,3-oxazole-4-carboxylic acid and 6-([4-[3-cyano-4-(piperidin-4-yloxy)phenyl]pyrimidin-2-yl]amino)-2-methoxy-N,N-dimethylpyridine-3-carboxamide using Method A. The final product was purified by prep-HPLC under the following conditions: column, XBridge Prep C18 Column, 150×19 mm, 5 um; mobile phase, acetonitrile in water (with 0.05% NH₃.H₂O), 35% to 43% gradient in 7 min; detector, UV 254 nm. 6-([4-[3-cyano-4-([1-[(1,3-oxazol-4-yl)carbonyl]piperidin-4-yl]oxy)phenyl]pyrimidin-2-yl]amino)-2-methoxy-N,N-dimethylpyridine-3-carboxamide was obtained as a white solid (34 mg, 30%). HPLC: 98.0% purity, RT=1.38 min. MS: m/z=569.1 [M+H]⁺. ¹H NMR (300 MHz, DMSO-d₆) δ 9.90 (s, 1H), 8.74-8.47 (m, 5H), 7.96-7.87 (m, 1H), 7.70-7.53 (m, 3H), 5.10-5.04 (m, 1H), 4.22-3.49 (m, 7H), 2.97 (s, 3H), 2.83 (s, 3H), 2.10-2.03 (m, 2H), 1.82-1.75 (m, 2H).

### Example 78: 6-([4-[3-cyano-4-([1-[(5-methyl-1H-1,2,4-triazol-3-yl)carbonyl]piperidin-4-yl]oxy)phenyl]pyrimidin-2-yl]amino)-2-methoxy-N,N-dimethylpyridine-3-carboxamide 78

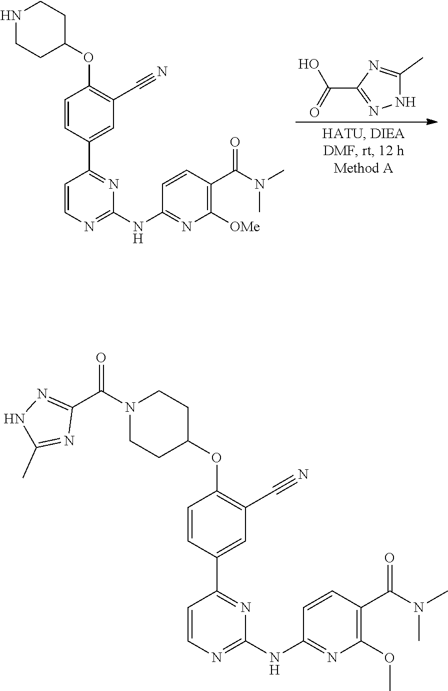

The title compound was prepared from 5-methyl-1H-1,2,4-triazole-3-carboxylic acid and 6-([4-[3-cyano-4-(piperidin-4-yloxy)phenyl]pyrimidin-2-yl]amino)-2-methoxy-N,N-dimethylpyridine-3-carboxamide using Method A. The final product was purified by prep-HPLC under the following conditions: column, XBridge Prep C18 Column, 150×19 mm, 5 um; mobile phase, acetonitrile in water (with 0.05% NH₃.H₂O), 25% to 49% gradient in 7 min; detector, UV 254 nm. 6-([4-[3-cyano-4-([1-[(5-methyl-1H-1,2,4-triazol-3-yl)carbonyl]piperidin-4-yl]oxy)phenyl]pyrimidin-2-yl]amino)-2-methoxy-N,N-dimethylpyridine-3-carboxamide was obtained as a white solid (36 mg, 31%). HPLC: 97.4% purity, RT=1.26 min. MS: m/z=583.3 [M+H]⁺. ¹H NMR (300 MHz, DMSO-d₆) δ 14.06 (s, 1H), 9.90 (s, 1H), 8.77-8.43 (m, 3H), 7.96-7.87 (m, 1H), 7.70-7.53 (m, 3H), 5.20-4.97 (m, 1H), 4.13-3.58 (m, 7H), 2.97 (s, 3H), 2.83 (s, 3H), 2.37 (s, 3H), 2.08-2.01 (m, 2H), 1.81-1.74 (m, 2H).

### Example 79: 6-([4-[3-cyano-4-([1-[(1,3-oxazol-5-yl)carbonyl]piperidin-4-yl]oxy)phenyl]pyrimidin-2-yl]amino)-2-methoxy-N,N-dimethylpyridine-3-carboxamide 79

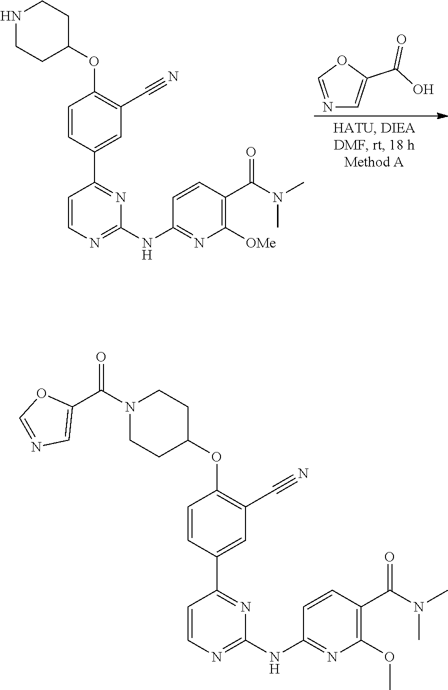 [SMILES: COc1nc(Nc2nccc(-c3ccc(OC4CCNCC4)c(C#N)c3)n2)ccc1C(=O)N(C)C ; O=C(O)c1cnco1 ; COc1nc(Nc2nccc(-c3ccc(OC4CCN(C(=O)c5cnco5)CC4)c(C#N)c3)n2)ccc1C(=O)N(C)C]

The title compound was prepared from 1,3-oxazole-5-carboxylic acid and 6-([4-[3-cyano-4-(piperidin-4-yloxy)phenyl]pyrimidin-2-yl]amino)-2-methoxy-N,N-dimethylpyridine-3-carboxamide using Method A. The final product was purified by prep-HPLC under the following conditions: column, XBridge Prep C18 Column, 150×19 mm, 5 um; mobile phase, acetonitrile in water (with 0.05% NH₃.H₂O), 35% to 42% gradient in 7 min; detector, UV 254 nm. 6-([4-[3-cyano-4-([1-[(1,3-oxazol-5-yl)carbonyl]piperidin-4-yl]oxy)phenyl]pyrimidin-2-yl]amino)-2-methoxy-N,N-dimethylpyridine-3-carboxamide was obtained as a white solid (35 mg, 17%). HPLC: 97.6% purity, RT=1.35 min. MS: m/z=569.3 [M+H]⁺. ¹H NMR (300 MHz, DMSO-d₆) δ 9.90 (s, 1H), 8.69-8.48 (m, 4H), 7.97-7.87 (m, 1H), 7.75 (s, 1H), 7.70-7.54 (m, 3H), 5.12-5.05 (m, 1H), 3.96-3.83 (m, 5H), 3.73-3.67 (m, 2H), 2.97 (s, 3H), 2.83 (s, 3H), 2.12-2.05 (m, 2H), 1.86-1.79 (m, 2H).

### Example 81: 2-[(1-methylazetidin-3-yl)oxy]-5-(2-[[5-(4-methylpiperazin-1-yl)pyridin-2-yl]amino]pyrimidin-4-yl)benzonitrile 81

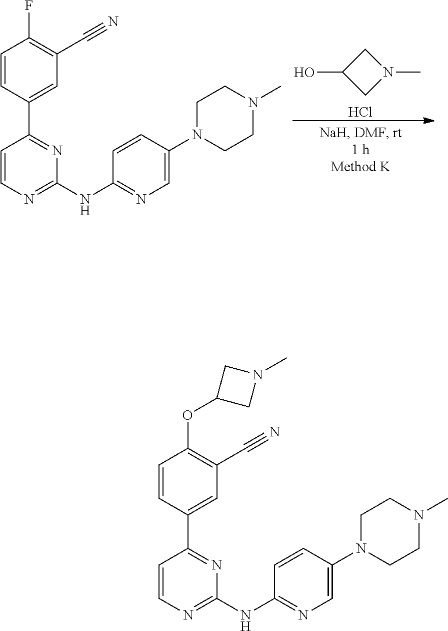

The title compound was prepared from 1-methylazetidin-3-ol hydrochloride and 2-fluoro-5-(2-[[5-(4-methylpiperazin-1-yl)pyridin-2-yl]amino]pyrimidin-4-yl)benzonitrile using Method K. The final product was purified by prep-HPLC under the following conditions: column, XBridge Prep C18 Column, 150×19 mm, 5 um; mobile phase, acetonitrile in water (with 10 mmol/L NR₄HCO₃ and 0.1% NH₃.H₂O), 34% to 35% gradient in 7 min; detector, UV 254 nm. 2-[(1-methylazetidin-3-yl)oxy]-5-(2-[[5-(4-methylpiperazin-1-yl)pyridin-2-yl]amino]pyrimidin-4-yl)benzonitrile was obtained as an yellow solid (18 mg, 17%). HPLC: 99.2% purity, RT=2.61 min. MS: m/z=457.2 [M+H]⁺. ¹H NMR (300 MHz, DMSO-d₆) δ 9.59 (s, 1H), 8.65-8.38 (m, 3H), 8.15-7.98 (m, 2H), 7.52-7.39 (m, 2H), 7.23-7.13 (m, 1H), 5.10-4.96 (m, 1H), 3.85-3.73 (m, 2H), 3.18-3.03 (m, 6H), 2.51-2.44 (m, 4H), 2.31 (s, 3H), 2.23 (S, 3H).

### Example 82: 5-(2-[[5-(4-methylpiperazin-1-yl)pyridin-2-yl]amino]pyrimidin-4-yl)-2-[(1-methylpyrrolidin-3-yl)oxy]benzonitrile hydrochloride 82

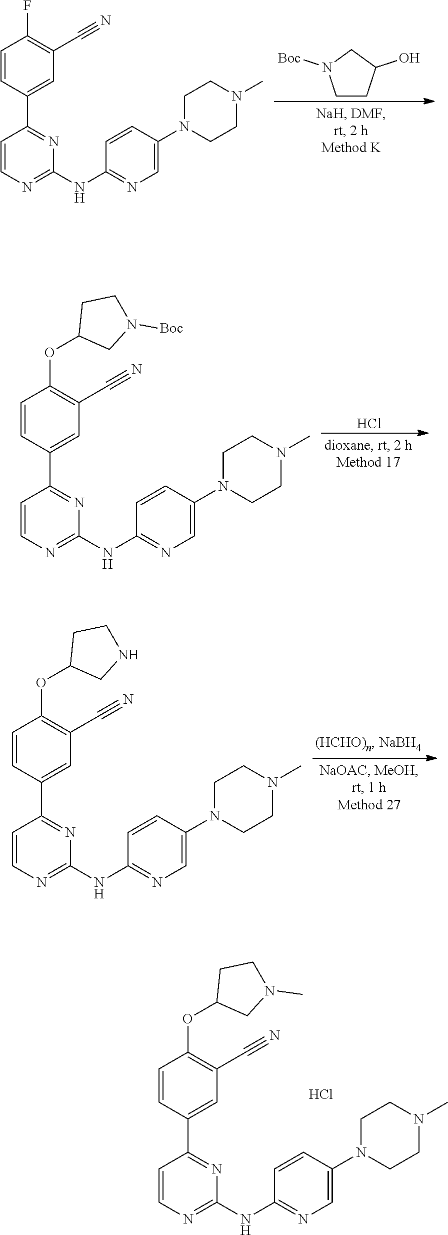

The title compound was prepared from 2-fluoro-5-(2-(5-(4-methylpiperazin-1-yl)pyridin-2-ylamino)pyrimidin-4-yl)benzonitrile, (HCHO)ₙ and tert-butyl 3-hydroxypyrrolidine-1-carboxylate using Method K, 17 and 27. The final product was purified by prep-HPLC under the following conditions: column, XBridge Prep C18 Column, 150×19 mm, 5 um; mobile phase, acetonitrile in water (with 0.05% HCl), 30% to 50% gradient in 7 min; detector, UV 254 nm. 5-(2-[[5-(4-methylpiperazin-1-yl)pyridin-2-yl]amino]pyrimidin-4-yl)-2-[(1-methylpyrrolidin-3-yl)oxy]benzonitrile hydrochloride was obtained as orange solid (25 mg, 13.3% for 3 steps). HPLC: 99.9% purity, RT=0.79 min. MS: m/z=471.1 [M+H]⁺. ¹H NMR (300 MHz, Methanol-d₄) δ 8.78-8.70 (m, 1H), 8.63-8.48 (m, 2H), 8.24-8.13 (m, 1H), 7.99-7.92 (m, 1H), 7.81-7.72 (m, 1H), 7.54-7.35 (m, 2H), 5.52-5.46 (m, 1H), 4.18-3.79 (m, 4H), 3.73-3.46 (m, 3H), 3.48-3.27 (m, 5H), 3.12 (s, 1.2H), 3.04 (s, 1.8H), 3.02 (s, 3H), 2.91-2.75 (m, 0.6H), 2.57-2.17 (m, 1.4H).

### Example 83: 5-(2-[[5-(4-methylpiperazin-1-yl)pyridin-2-yl]amino]pyrimidin-4-yl)-2-[(1-methylpiperidin-4-yl)oxy]benzonitrile 83

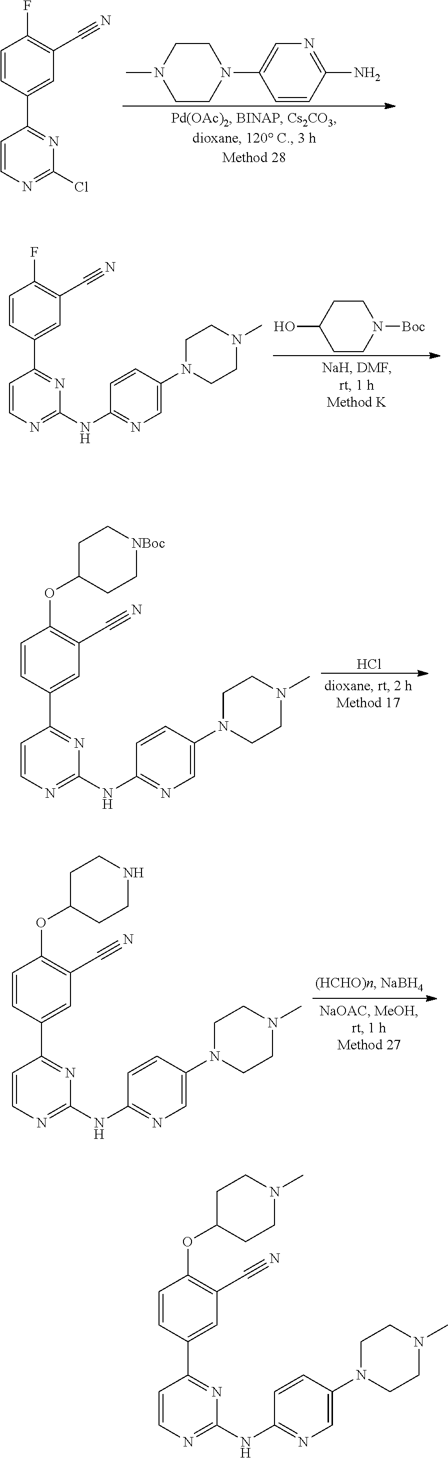

The title compound was prepared from 5-(2-chloropyrimidin-4-yl)-2-fluorobenzonitrile, 5-(4-methylpiperazin-1-yl)pyridin-2-amine, tert-butyl 4-hydroxypiperidine-1-carboxylate, and POM using Methods 28, K, 27, and 17. The final product was purified by prep-HPLC under the following conditions: column, XBridge Prep C18 Column, 150×19 mm, 5 um; mobile phase, acetonitrile in water (with 10 mmol/L NH₁HCO₃ and 0.1% NH₃.H₂O), 35% to 36% gradient in 7 min; detector, UV 254 nm. 5-(2-[[5-(4-methylpiperazin-1-yl)pyridin-2-yl]amino]pyrimidin-4-yl)-2-[(1-methylpiperidin-4-yl)oxy]benzonitrile was obtained as an yellow solid (25 mg, 4% for 4 steps). HPLC: 99.8% purity, RT=2.08 min. MS: m/z=485.1 [M+H]⁺. ¹H NMR (300 MHz, DMSO-d₆) δ 9.59 (s, 1H), 8.58-8.41 (m, 3H), 8.16-7.97 (m, 2H), 7.53-7.39 (m, 3H), 4.79-4.70 (m, 1H), 3.18-3.08 (m, 4H), 2.67-2.38 (m, 6H), 2.37-2.13 (m, 8H), 2.06-1.89 (m, 2H), 1.83-1.64 (m, 2H).

### Example 84: 2-[[(3R,4S)-3-fluoro-1-methylpiperidin-4-yl]oxy]-5-(2-[[5-(4-methylpiperazin-1-yl)pyridin-2-yl]amino]pyrimidin-4-yl)benzonitrile 84

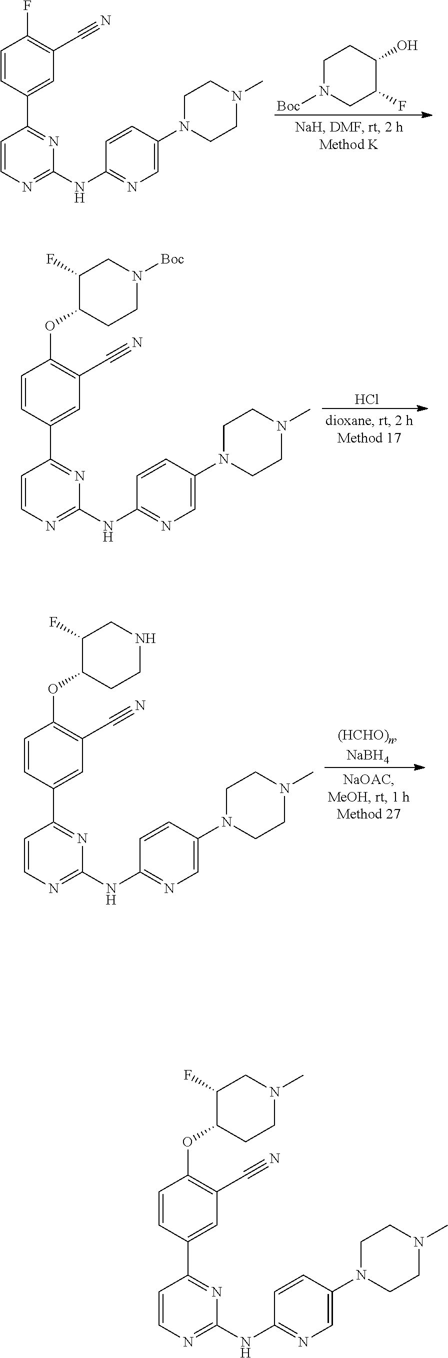

The title compound was prepared from 2-fluoro-5-(2-(5-(4-methylpiperazin-1-yl)pyridin-2-ylamino)pyrimidin-4-yl)benzonitrile, (3R,4S)-tert-butyl 3-fluoro-4-hydroxypiperidine-1-carboxylate, and POM using Method K, 17, and 27. The final product was purified by prep-HPLC under the following conditions: column, XBridge Prep C18 Column, 150×19 mm, 5 um; mobile phase, acetonitrile in water (with 10 mmol/L NH₁HCO₃ and 0.1% NH₃.H₂O), 34% to 35% gradient in 7 min; detector, UV 254 nm. 2-[[(3R,4S)-3-fluoro-1-methylpiperidin-4-yl]oxy]-5-(2-[[5-(4-methylpiperazin-1-yl)pyridin-2-yl]amino]pyrimidin-4-yl)benzonitrile was obtained as brown solid (26 mg, 8.6% for 3 steps). HPLC: 99.3% purity, RT=3.09 min. MS: m/z=503.3 [M+H]⁺. ¹H NMR (400 MHz, DMSO-d₆) δ 9.57 (s, 1H), 8.58-8.52 (m, 2H), 8.49-8.42 (m, 1H), 8.10 (d, J=9.0 Hz, 1H), 8.04-7.98 (m, 1H), 7.61-7.41 (m, 3H), 5.09-4.82 (m, 2H), 3.16-3.09 (m, 4H), 2.89-2.56 (m, 3H), 2.51-2.43 (m, 4H), 2.36-2.31 (m, 1H), 2.27-2.20 (m, 6H), 2.10-1.82 (m, 2H).

### Example 85: 2-[(3,3-difluoro-1-methylpiperidin-4-yl)oxy]-5-(2-[[5-(4-methylpiperazin-1-yl)pyridin-2-yl]amino]pyrimidin-4-yl)benzonitrile 85

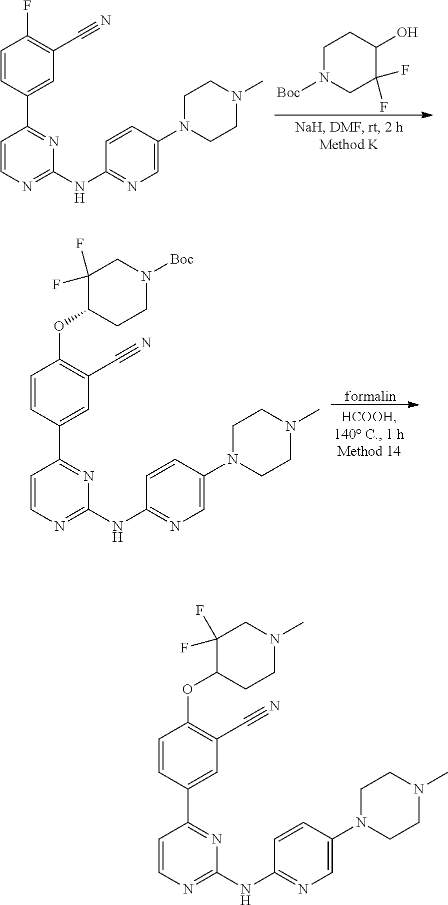

The title compound was prepared from 2-fluoro-5-(2-(5-(4-methylpiperazin-1-yl)pyridin-2-ylamino)pyrimidin-4-yl)benzonitrile, tert-butyl 3,3-difluoro-4-hydroxypiperidine-1-carboxylate, and formalin using Method K and 14. The final product was purified by prep-HPLC under the following conditions: column, XBridge Prep C18 Column, 150×19 mm, 5 um; mobile phase, acetonitrile in water (with 10 mmol/L NH₄HCO₃ and 0.1% NH₃.H₂O), 30% to 50% gradient in 7 min; detector, UV 254 nm. 2-[(3,3-difluoro-1-methylpiperidin-4-yl)oxy]-5-(2-[[5-(4-methylpiperazin-1-yl)pyridin-2-yl]amino]pyrimidin-4-yl)benzonitrile was obtained as brown solid (22 mg, 16% for 2 steps). HPLC: 92.1% purity, RT=1.57 min. MS: m/z=521.2 [M+H]⁺. ¹H NMR (300 MHz, DMSO-d₆) δ 9.66 (s, 1H), 8.70-8.40 (m, 3H), 8.16-8.00 (m, 2H), 7.75-7.34 (m, 3H), 5.22-5.06 (m, 1H), 3.18-3.08 (m, 4H), 3.01-2.38 (m, 8H), 2.32-2.20 (m, 6H), 2.14-1.86 (m, 2H).

### Example 86: 5-(2-[[6-methoxy-5-(4-methylpiperazin-1-yl)pyridin-2-yl]amino]pyrimidin-4-yl)-2-[(1-methylazetidin-3-yl)oxy]benzonitrile 86

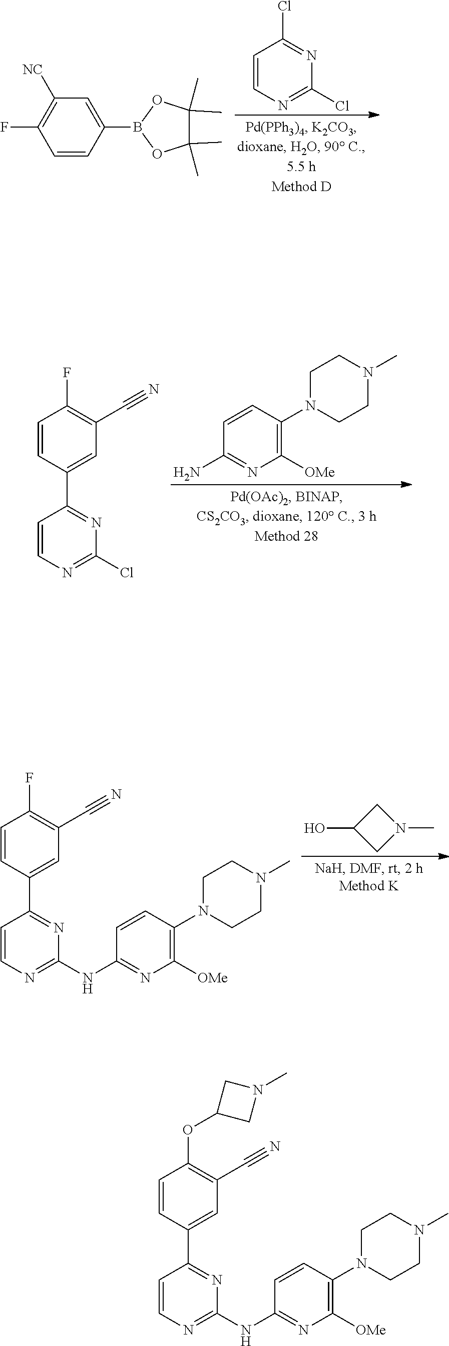

The title compound was prepared from 2-fluoro-5-(4,4,5,5-tetramethyl-1,3,2-dioxaborolan-2-yl)benzonitrile, 2,4-dichloropyrimidine, 6-methoxy-5-(4-methylpiperazin-1-yl)pyridin-2-amine, and 1-methylazetidin-3-ol using Method D, 28, and K. The final product was purified by prep-HPLC under the following conditions: column, XBridge Shield RP18 OBD Column, 150×19 mm, 5 um; mobile phase, acetonitrile in water (with 0.05% NH₃.H₂O), 40% to 60% gradient in 8 min; detector, UV 254 nm. 5-(2-[[6-methoxy-5-(4-methylpiperazin-1-yl)pyridin-2-yl]amino]pyrimidin-4-yl)-2-[(1-methylazetidin-3-yl)oxy]benzonitrile was obtained as an yellow solid (11 mg, 2.8% for 3 steps). HPLC: 90.9% purity, RT=1.84 min. MS: m/z=487.2 [M+H]⁺. ¹H NMR (300 MHz, Methanol-d₄) δ 8.57-8.38 (m, 3H), 7.94-7.85 (m, 1H), 7.40-7.31 (m, 2H), 7.14-7.04 (m, 1H), 5.18-5.11 (m, 1H), 4.20-4.07 (m, 2H), 3.99 (s, 3H), 3.70-3.63 (m, 2H), 3.22-3.15 (m, 4H), 3.05-2.99 (m, 4H), 2.68-2.59 (m, 6H).

### Example 87: 5-(2-[[6-methoxy-5-(4-methylpiperazin-1-yl)pyridin-2-yl]amino]pyrimidin-4-yl)-2-[(1-methylpyrrolidin-3-yl)oxy]benzonitrile 87

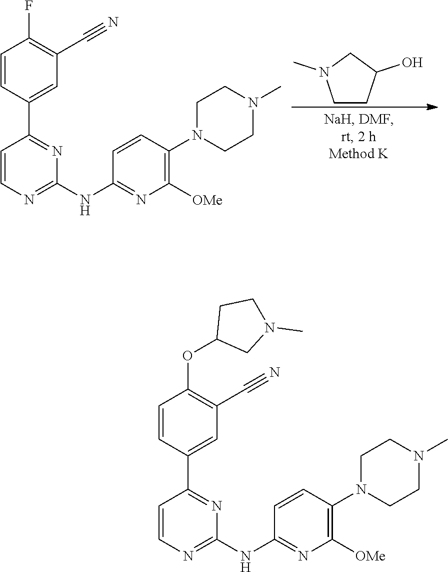

The title compound was prepared from 1-methylpyrrolidin-3-ol and 2-fluoro-5-(2-(6-methoxy-5-(4-methylpiperazin-1-yl)pyridin-2-ylamino)pyrimidin-4-yl)benzonitrile using Method K. The final product was purified by prep-HPLC under the following conditions: column, XBridge Prep OBD C18 Column, 150×19 mm, 5 um; mobile phase, acetonitrile in water (with 0.05% NH₃.H₂O), 35% to 65% gradient in 8 min; detector, UV 254 nm. 5-(2-[[6-methoxy-5-(4-methylpiperazin-1-yl)pyridin-2-yl]amino]pyrimidin-4-yl)-2-[(1-methylpyrrolidin-3-yl)oxy]benzonitrile was obtained as a yellow solid (16 mg, 28%). HPLC: 97.7% purity, RT=1.01 min. MS: m/z=501.2 [M+H]⁺. ¹H NMR (300 MHz, Methanol-d₄) δ 8.52-8.36 (m, 3H), 7.86 (d, J=8.3 Hz, 1H), 7.36-7.18 (m, 2H), 5.16-5.09 (m, 1H), 3.96 (s, 3H), 3.15-2.79 (m, 7H), 2.71-2.45 (m, 6H), 2.40 (s, 3H), 2.34 (s, 3H), 2.11-2.01 (m, 1H).

### Example 88: 5-(2-[[6-methoxy-5-(4-methylpiperazin-1-yl)pyridin-2-yl]amino]pyrimidin-4-yl)-2-[(1-methylpiperidin-4-yl)oxy]benzonitrile 88

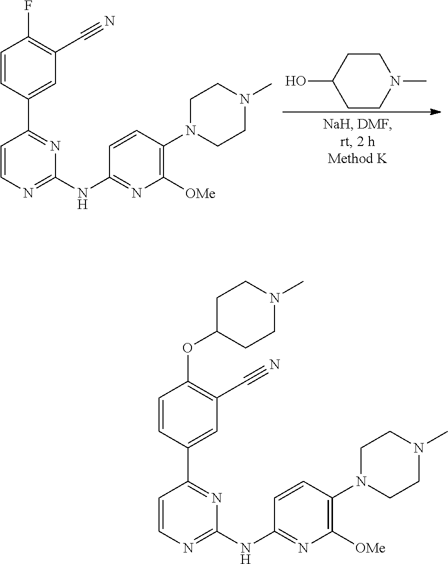

The title compound was prepared from 1-methylpiperidin-4-ol and 2-fluoro-5-(2-(6-methoxy-5-(4-methylpiperazin-1-yl)pyridin-2-ylamino)pyrimidin-4-yl)benzonitrile using Method K. The final product was purified by prep-HPLC under the following conditions: column, XBridge Prep OBD C18 Column, 150×19 mm, 5 um; mobile phase, acetonitrile in water (with 0.05% NH₃.H₂O), 35% to 65% gradient in 8 min; detector, UV 254 nm. 5-(2-[[6-methoxy-5-(4-methylpiperazin-1-yl)pyridin-2-yl]amino]pyrimidin-4-yl)-2-[(1-methylpiperidin-4-yl)oxy]benzonitrile was obtained as an yellow solid (13 mg, 22%). HPLC: 96.2% purity, RT=2.04 min. MS: m/z=515.2 [M+H]⁺. ¹H NMR (300 MHz, Methanol-d₄) δ 8.52-8.35 (m, 3H), 7.86 (d, J=8.4 Hz, 1H), 7.40-7.27 (m, 3H), 4.77 (br s, 1H), 3.96 (s, 3H), 3.08-3.02 (m, 4H), 2.83-2.41 (m, 8H), 2.34 (br s, 6H), 2.09-2.03 (m, 2H), 1.98-1.91 (m, 2H).

### Example 89: 5-(2-[[6-methoxy-5-(4-methylpiperazin-1-yl)pyridin-2-yl]amino]pyrimidin-4-yl)-2-(piperidin-4-yloxy)benzonitrile 89

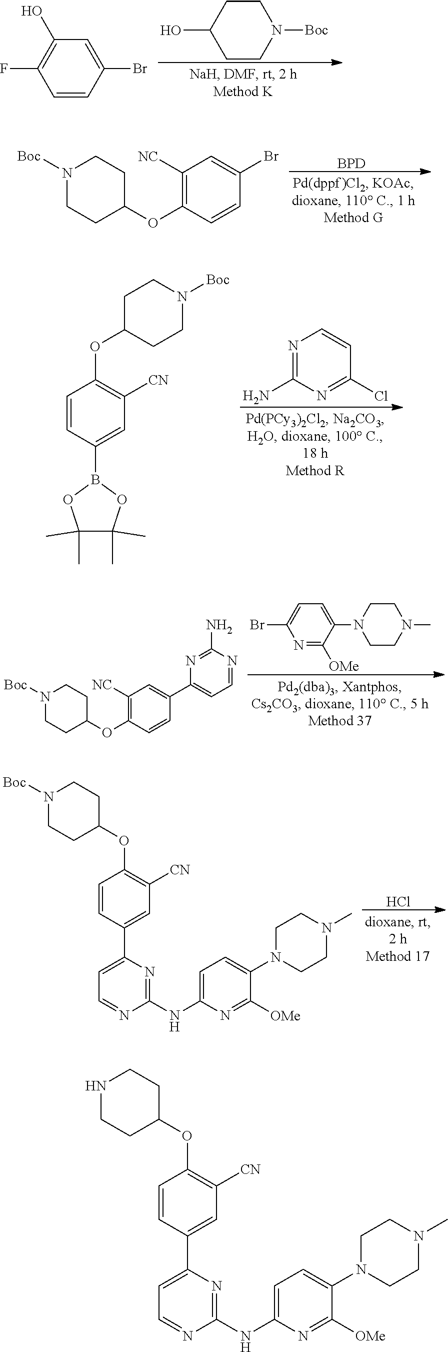

The title compound was prepared from 5-bromo-2-fluorobenzonitrile, tert-butyl 4-hydroxypiperidine-1-carboxylate, BPD, 4-chloropyrimidin-2-amine and 1-(6-bromo-2-methoxypyridin-3-yl)-4-methylpiperazine using Method K, G, R, 37, and 17. The final product was purified by prep-HPLC under the following conditions: column, XBridge Prep C18 Column, 150×19 mm, 5 um; mobile phase, acetonitrile in water (with 0.05% NH₃.H₂O), 34% to 36% gradient in 7 min; detector, UV 254 nm. 5-(2-[[6-methoxy-5-(4-methylpiperazin-1-yl)pyridin-2-yl]amino]pyrimidin-4-yl)-2-(piperidin-4-yloxy)benzonitrile was obtained as an yellow solid (25 mg, 13.6% for 5 steps). HPLC: 98.5% purity, RT=0.71 min. MS: m/z=501.2 [M+H]⁺. ¹H NMR (300 MHz, Methanol-d₄) δ 8.51-8.34 (m, 3H), 7.90-7.80 (m, 1H), 7.40-7.26 (m, 3H), 4.85-4.71 (m, 1H), 3.95 (s, 3H), 3.22-2.97 (m, 6H), 2.89-2.73 (m, 2H), 2.64-2.58 (m, 4H), 2.33 (s, 3H), 2.13-2.00 (m, 2H), 1.89-1.74 (m, 2H).

### Example 90: 2-[[1-(2-hydroxyacetyl)piperidin-4-yl]oxy]-5-(2-[[6-methoxy-5-(4-methylpiperazin-1-yl)pyridin-2-yl]amino]pyrimidin-4-yl)benzonitrile 90

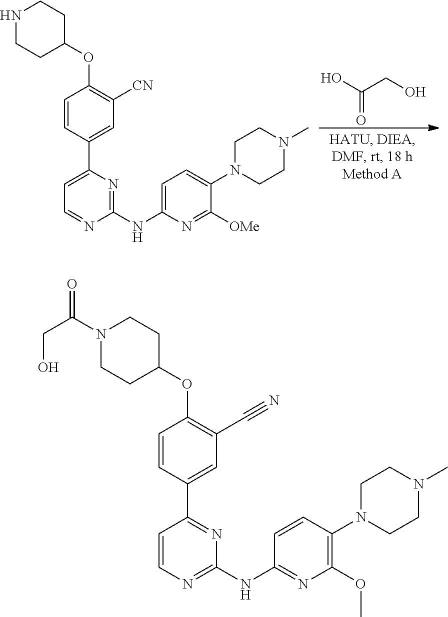 [SMILES: COc1nc(Nc2nccc(-c3ccc(OC4CCNCC4)c(C#N)c3)n2)ccc1N1CCN(C)CC1 ; O=C(O)CO ; COc1nc(Nc2nccc(-c3ccc(OC4CCN(C(=O)CO)CC4)c(C#N)c3)n2)ccc1N1CCN(C)CC1]

The title compound was prepared from 5-(2-[[6-methoxy-5-(4-methylpiperazin-1-yl)pyridin-2-yl]amino]pyrimidin-4-yl)-2-(piperidin-4-yloxy)benzonitrile and 2-hydroxyacetic acid using Method A. The final product was purified by prep-HPLC under the following conditions: column, XBridge Prep C18 Column, 150×19 mm, 5 um; mobile phase, acetonitrile in water (with 0.05% NH₃.H₂O), 39% to 41% gradient in 7 min; detector, UV 254 nm. 2-[[1-(2-hydroxyacetyl)piperidin-4-yl]oxy]-5-(2-[[6-methoxy-5-(4-methylpiperazin-1-yl)pyridin-2-yl]amino]pyrimidin-4-yl)benzonitrile was obtained as an yellow solid (24 mg, 23%). HPLC: 93.5% purity, RT=2.50 min. MS: m/z=559.1 [M+H]⁺. ¹H NMR (300 MHz, DMSO-d₆) δ 9.35 (s, 1H), 8.60-8.43 (m, 3H), 7.76-7.67 (m, 1H), 7.59-7.46 (m, 2H), 7.29-7.19 (m, 1H), 5.02-4.96 (m, 1H), 4.60-4.50 (m, 1H), 4.15-4.07 (m, 2H), 3.88 (s, 3H), 3.82-3.35 (m, 4H), 2.96-2.90 (m, 4H), 2.47-2.40 (m, 4H), 2.20 (s, 3H), 2.10-1.56 (m, 4H).

### Example 91: 2-[[1-(2-hydroxypropanoyl)piperidin-4-yl]oxy]-5-(2-[[6-methoxy-5-(4-methylpiperazin-1-yl)pyridin-2-yl]amino]pyrimidin-4-yl)benzonitrile hydrochloride 91

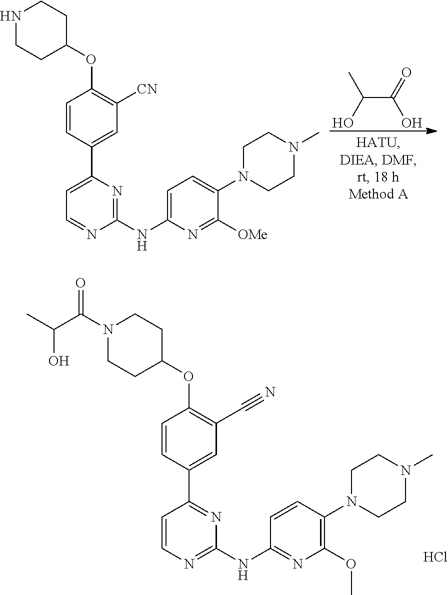

The title compound was prepared from 5-(2-[[6-methoxy-5-(4-methylpiperazin-1-yl)pyridin-2-yl]amino]pyrimidin-4-yl)-2-(piperidin-4-yloxy)benzonitrile and 2-hydroxypropanoic acid using Method A. The final product was purified by prep-HPLC under the following conditions: column, XBridge Prep C18 Column, 150×19 mm, 5 um; mobile phase, acetonitrile in water (with 0.05% HCl), 25% to 55% gradient in 7 min; detector, UV 254 nm. 2-[[1-(2-hydroxypropanoyl)piperidin-4-yl]oxy]-5-(2-[[6-methoxy-5-(4-methylpiperazin-1-yl)pyridin-2-yl]amino]pyrimidin-4-yl)benzonitrile hydrochloride was obtained as orange solid (14 mg, 12%). HPLC: 95.2% purity, RT=4.23 min. MS: m/z=573.2 [M+H]⁺. ¹H NMR (300 MHz, Methanol-d₄) δ 8.77-8.60 (m, 3H), 7.93-7.84 (m, 1H), 7.63-7.52 (m, 2H), 6.99-6.90 (m, 1H), 5.15-5.09 (m, 1H), 4.22 (s, 3H), 4.08-3.51 (m, 9H), 3.42-3.32 (m, 2H), 3.23-3.08 (m, 2H), 3.00 (s, 3H), 2.31-1.76 (m, 4H), 1.43-1.36 (m, 3H).

### Example 92: 5-(2-[[6-methoxy-5-(4-methylpiperazin-1-yl)pyridin-2-yl]amino]pyrimidin-4-yl)-2-([1-[(1,3-oxazol-5-yl)carbonyl]piperidin-4-yl]oxy)benzonitrile 92

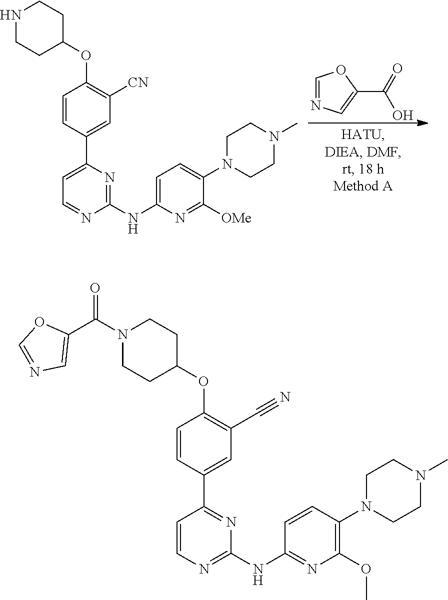

The title compound was prepared from 5-(2-[[6-methoxy-5-(4-methylpiperazin-1-yl)pyridin-2-yl]amino]pyrimidin-4-yl)-2-(piperidin-4-yloxy)benzonitrile and 1,3-oxazole-5-carboxylic acid using Method A. The final product was purified by prep-HPLC under the following conditions: column, XBridge Prep C18 Column, 150×19 mm, 5 um; mobile phase, acetonitrile in water (with 0.05% NH₃.H₂O), 39% to 40% gradient in 7 min; detector, UV 254 nm. 5-(2-[[6-methoxy-5-(4-methylpiperazin-1-yl)pyridin-2-yl]amino]pyrimidin-4-yl)-2-([1-[(1,3-oxazol-5-yl)carbonyl]piperidin-4-yl]oxy)benzonitrile was obtained as an yellow solid (24 mg, 26%). HPLC: 98.1% purity, RT=2.74 min. MS: m/z=596.1 [M+H]⁺. ¹H NMR (300 MHz, Methanol-d₄) δ 8.53-8.39 (m, 3H), 8.33 (s, 1H), 7.91-7.81 (m, 1H), 7.67 (s, 1H), 7.47-7.27 (m, 4H), 5.10-4.97 (m, 1H), 3.99-3.87 (m, 7H), 3.20-2.94 (m, 5H), 2.70-2.50 (m, 4H), 2.33 (s, 3H), 2.20-1.85 (m, 4H).

### Example 93: 5-(2-[[6-methoxy-5-(4-methylpiperazin-1-yl)pyridin-2-yl]amino]pyrimidin-4-yl)-2-([1-[(1,3-oxazol-4-yl)carbonyl]piperidin-4-yl]oxy)benzonitrile 93

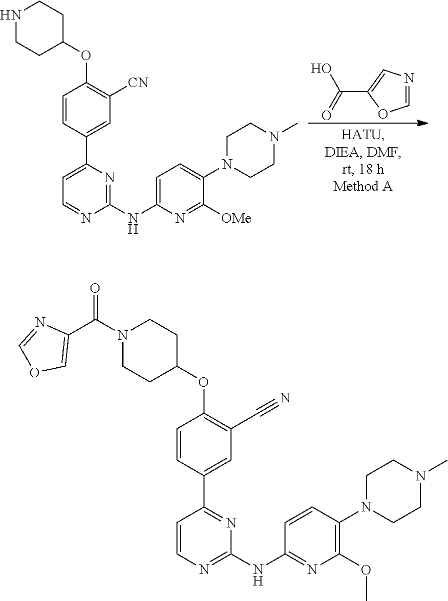

The title compound was prepared from 5-(2-[[6-methoxy-5-(4-methylpiperazin-1-yl)pyridin-2-yl]amino]pyrimidin-4-yl)-2-(piperidin-4-yloxy)benzonitrile and 1,3-oxazole-4-carboxylic acid using Method A. The final product was purified by prep-HPLC under the following conditions: column, XBridge Prep C18 Column, 150×19 mm, 5 um; mobile phase, acetonitrile in water (with 0.05% NH₃.H₂O), 42% to 42% gradient in 7 min; detector, UV 254 nm. 5-(2-[[6-methoxy-5-(4-methylpiperazin-1-yl)pyridin-2-yl]amino]pyrimidin-4-yl)-2-([1-[(1,3-oxazol-4-yl)carbonyl]piperidin-4-yl]oxy)benzonitrile was obtained as an yellow solid (27 mg, 24%). HPLC: 97.3% purity, RT=2.79 min. MS: m/z=596.1 [M+H]⁺. ¹H NMR (300 MHz, Methanol-d₄) δ 8.53-8.32 (m, 5H), 8.26-8.19 (m, 1H), 7.91-7.81 (m, 1H), 7.46-7.27 (m, 4H), 5.05-4.98 (m, 1H), 4.24-3.78 (m, 7H), 3.18-2.89 (m, 4H), 2.64-2.58 (m, 4H), 2.33 (s, 3H), 2.21-1.82 (m, 4H).

### Example 94: 5-(2-[[6-methoxy-5-(4-methylpiperazin-1-yl)pyridin-2-yl]amino]pyrimidin-4-yl)-2-([1-[(5-methyl-1H-1,2,4-triazol-3-yl)carbonyl]piperidin-4-yl]oxy)benzonitrile 94

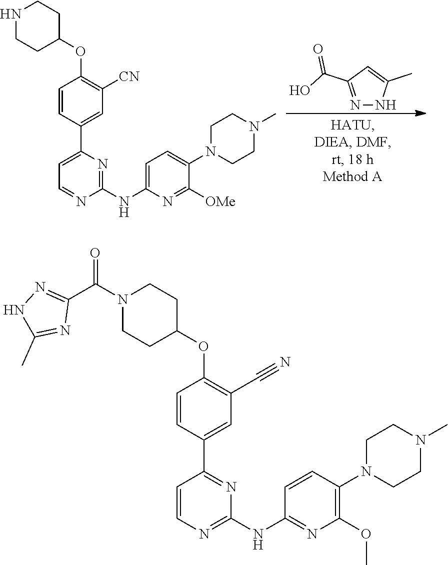

The title compound was prepared from 5-(2-[[6-methoxy-5-(4-methylpiperazin-1-yl)pyridin-2-yl]amino]pyrimidin-4-yl)-2-(piperidin-4-yloxy)benzonitrile and 5-methyl-1H-1,2,4-triazole-3-carboxylic acid using Method A. The final product was purified by prep-HPLC under the following conditions: column, XBridge Prep C18 Column, 150×19 mm, 5 um; mobile phase, acetonitrile in water (with 0.05% NH₃.H₂O), 33% to 37% gradient in 7 min; detector, UV 254 nm. 5-(2-[[6-methoxy-5-(4-methylpiperazin-1-yl)pyridin-2-yl]amino]pyrimidin-4-yl)-2-([1-[(5-methyl-1H-1,2,4-triazol-3-yl)carbonyl]piperidin-4-yl]oxy)benzonitrile was obtained as an yellow solid (34 mg, 15%). HPLC: 98.1% purity, RT=1.07 min. MS: m/z=610.4 [M+H]⁺. ¹H NMR (300 MHz, DMSO-d₆) δ 14.09 (s, 1H), 9.40 (s, 1H), 8.67-8.46 (m, 3H), 7.79-7.70 (m, 1H), 7.62-7.49 (m, 2H), 7.32-7.23 (m, 1H), 5.10-5.03 (m, 1H), 4.16-3.55 (m, 7H), 2.98 (br s, 4H), 2.59-2.52 (m, 4H), 2.38 (s, 3H), 2.29 (s, 3H), 2.08-2.02 (m, 2H), 1.87-1.65 (m, 2H).

### Example 95: tert-butyl (3R,4S)-4-[2-cyano-4-(2-[[6-methoxy-5-(4-methylpiperazin-1-yl)pyridin-2-yl]amino]pyrimidin-4-yl)phenoxy]-3-fluoropiperidine-1-carboxylate 95

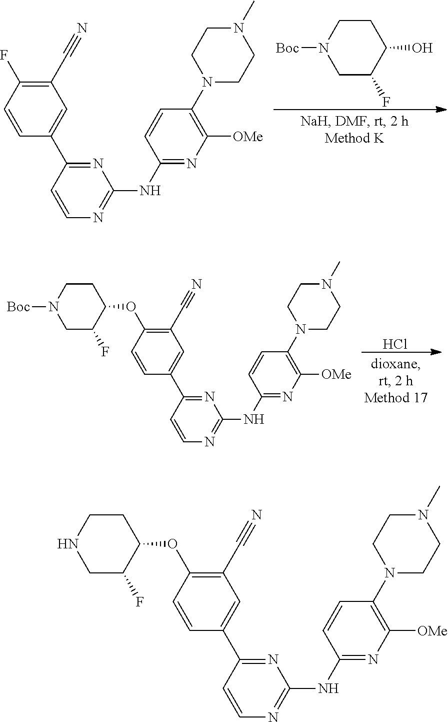

The title compound was prepared from 2-fluoro-5-(2-(6-methoxy-5-(4-methylpiperazin-1-yl)pyridin-2-ylamino)pyrimidin-4-yl)benzonitrile and (3R,4S)-tert-butyl 3-fluoro-4-hydroxypiperidine-1-carboxylate using Method K and 17. The final product was purified by prep-HPLC under the following conditions: column, XBridge Prep C18 Column, 150×19 mm, 5 um; mobile phase, acetonitrile in water (with 10 mmol/L NH₁HCO₃ and 0.1% NH₃.H₂O), 50% to 70% gradient in 8 min; detector, UV 254 nm. tert-butyl (3R,4S)-4-[2-cyano-4-(2-[[6-methoxy-5-(4-methylpiperazin-1-yl)pyridin-2-yl]amino]pyrimidin-4-yl)phenoxy]-3-fluoropiperidine-1-carboxylate was obtained as a light yellow solid (63 mg, 33% for 2 steps). HPLC: 98.2% purity, RT=0.93 min. MS: m/z=519.6 [M+H]⁺. ¹H NMR (300 MHz, Methanol-d₄) δ 8.49-8.31 (m, 3H), 7.87-7.77 (m, 1H), 7.45-7.21 (m, 3H), 5.05-4.80 (m, 2H), 3.93 (s, 3H), 3.38-3.27 (m, 1H), 3.15-2.90 (m, 6H), 2.85-2.57 (m, 5H), 2.38 (s, 3H), 2.18-1.82 (m, 2H).

### Example 96: 2-(((3R,4S)-3-fluoro-1-methylpiperidin-4-yl)oxy)-5-(2-((6-methoxy-5-(4-methylpiperazin-1-yl)pyridin-2-yl)amino)pyrimidin-4-yl)benzonitrile 96

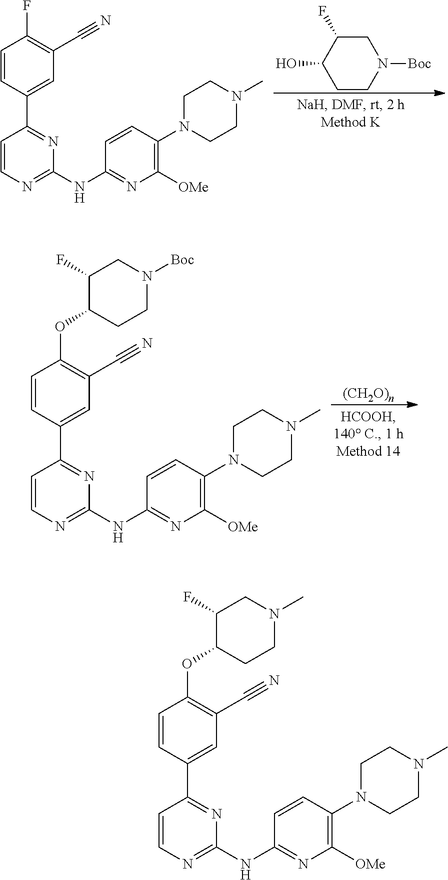

The title compound was prepared from 2-fluoro-5-(2-(6-methoxy-5-(4-methylpiperazin-1-yl)pyridin-2-ylamino)pyrimidin-4-yl)benzonitrile, (3R)-tert-butyl 3-fluoro-4-hydroxypiperidine-1-carboxylate and (HCHO)ₙ using Method K and 14. The final product was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 35% to 47% gradient in 8 min; detector, UV 254 nm. 2-(((3R,4S)-3-fluoro-1-methylpiperidin-4-yl)oxy)-5-(2-((6-methoxy-5-(4-methylpiperazin-1-yl)pyridin-2-yl)amino)pyrimidin-4-yl)benzonitrile was obtained as an yellow solid (29 mg, 17% for 2 steps). HPLC: 97.6% purity, RT=2.90 min. MS: m/z=553.2 [M+H]⁺. ¹H NMR (300 MHz, Methanol-d₄) δ 8.52-8.34 (m, 3H), 7.89-7.80 (m, 1H), 7.44-7.26 (m, 3H), 4.99-4.93 (m, 2H), 3.95 (s, 3H), 3.16-2.81 (m, 5H), 2.79-2.42 (m, 7H), 2.35 (s, 3H), 2.33 (s, 3H), 2.24-1.85 (m, 2H).

### Example 97: 2-[(3,3-difluoropiperidin-4-yl)oxy]-5-(2-[[6-methoxy-5-(4-methylpiperazin-1-yl)pyridin-2-yl]amino]pyrimidin-4-yl)benzonitrile 97

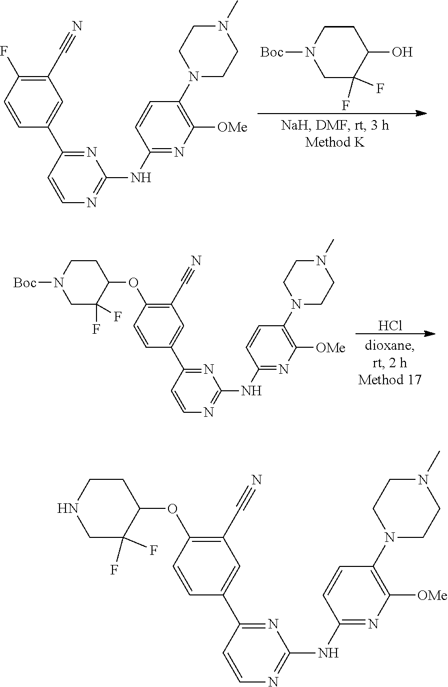 [SMILES: COc1nc(Nc2nccc(-c3ccc(F)c(C#N)c3)n2)ccc1N1CCN(C)CC1 ; CC(C)(C)OC(=O)N1CCC(O)C(F)(F)C1 ; COc1nc(Nc2nccc(-c3ccc(OC4CCN(C(=O)OC(C)(C)C)CC4(F)F)c(C#N)c3)n2)ccc1N1CCN(C)CC1 ; Cl ; COc1nc(Nc2nccc(-c3ccc(OC4CCNCC4(F)F)c(C#N)c3)n2)ccc1N1CCN(C)CC1]

The title compound was prepared from 2-fluoro-5-(2-(6-methoxy-5-(4-methylpiperazin-1-yl)pyridin-2-ylamino)pyrimidin-4-yl)benzonitrile and tert-butyl 3,3-difluoro-4-hydroxypiperidine-1-carboxylate using Method K and 17. The final product was purified by prep-HPLC under the following conditions: column, XBridge Prep C18 Column, 150×19 mm, 5 um; mobile phase, acetonitrile in water (with 10 mmol/L NH₁HCO₃ and 0.1% NH₃.H₂O), 37% to 55% gradient in 8 min; detector, UV 254 nm. 2-[(3,3-difluoropiperidin-4-yl)oxy]-5-(2-[[6-methoxy-5-(4-methylpiperazin-1-yl)pyridin-2-yl]amino]pyrimidin-4-yl)benzonitrile was obtained as a white solid (37 mg, 22% for 2 steps). HPLC: 99.3% purity, RT=2.03 min. MS: m/z=537.6 [M+H]⁺. ¹H NMR (300 MHz, Methanol-d₄) δ 8.56-8.35 (m, 3H), 7.94-7.84 (m, 1H), 7.52-7.28 (m, 3H), 5.12-5.05 (m, 1H), 3.97 (s, 3H), 3.39-3.28 (m, 8H), 3.27-3.04 (m, 3H), 2.93-2.87 (m, 4H), 2.17-2.11 (m, 2H).

### Example 98: 2-[(3,3-difluoro-1-methylpiperidin-4-yl)oxy]-5-(2-[[6-methoxy-5-(4-methylpiperazin-1-yl)pyridin-2-yl]amino]pyrimidin-4-yl)benzonitrile 98

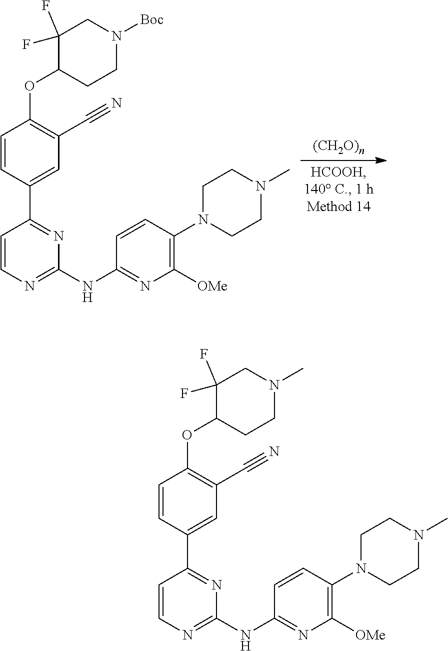 [SMILES: COc1nc(Nc2nccc(-c3ccc(OC4CCN(C(=O)OC(C)(C)C)CC4(F)F)c(C#N)c3)n2)ccc1N1CCN(C)CC1 ; COc1nc(Nc2nccc(-c3ccc(OC4CCN(C)CC4(F)F)c(C#N)c3)n2)ccc1N1CCN(C)CC1]

### Method 14

To a solution of tert-butyl 4-[2-cyano-4-(2-{[6-methoxy-5-(4-methylpiperazin-1-yl)pyridin-2-yl]amino}pyrimidin-4-yl)phenoxy]-3,3-difluoropiperidine-1-carboxylate in HCOOH (10 mL) was added formalin (100 equiv) at room temperature. The resulting mixture was stirred for 1.5 h at 140° C. When the reaction was done, the reaction mixture was concentrated under reduced pressure and the residue was purified by prep-HPLC under the following conditions: column, XBridge Shield RP18 OBD Column, 150×19 mm, 5 um; mobile phase, acetonitrile in water (with 10 mmol/L NH₄HCO₃ and 0.1% NH₃.H₂O), 38% to 45% gradient in 8 min; detector, UV 254 nm. 2-[(3,3-difluoro-1-methylpiperidin-4-yl)oxy]-5-(2-[[6-methoxy-5-(4-methylpiperazin-1-yl)pyridin-2-yl]amino]pyrimidin-4-yl)benzonitrile was obtained as an yellow solid (29 mg, 59%). HPLC: 96.0% purity, RT=3.03 min. MS: m/z=551.2 [M+H]⁺. ¹H NMR (300 MHz, Methanol-d₄) δ 8.51-8.33 (m, 3H), 7.87-7.77 (m, 1H), 7.48-7.38 (m, 1H), 7.34-7.24 (m, 2H), 5.00-4.89 (m, 1H), 3.95 (s, 3H), 3.13-2.76 (m, 6H), 2.68-2.50 (m, 6H), 2.37 (s, 3H), 2.32 (s, 3H), 2.16-2.10 (m, 2H).

### Example 99: 2-[[3,3-difluoro-1-(2-hydroxyacetyl)piperidin-4-yl]oxy]-5-(2-[[6-methoxy-5-(4-methylpiperazin-1-yl)pyridin-2-yl]amino]pyrimidin-4-yl)benzonitrile 99

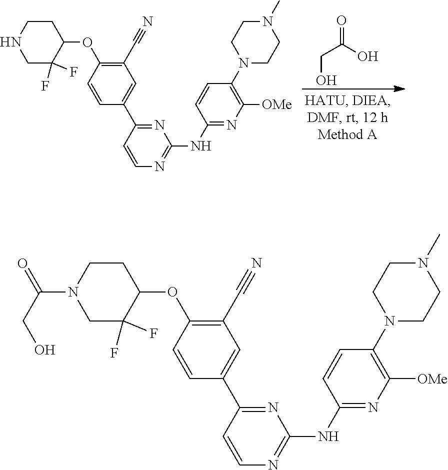 [SMILES: COc1nc(Nc2nccc(-c3ccc(OC4CCNCC4(F)F)c(C#N)c3)n2)ccc1N1CCN(C)CC1 ; O=C(O)CO ; COc1nc(Nc2nccc(-c3ccc(OC4CCN(C(=O)CO)CC4(F)F)c(C#N)c3)n2)ccc1N1CCN(C)CC1]

The title compound was prepared from 2-[(3,3-difluoropiperidin-4-yl)oxy]-5-(2-[[6-methoxy-5-(4-methylpiperazin-1-yl)pyridin-2-yl]amino]pyrimidin-4-yl)benzonitrile and 2-hydroxyacetic acid using Method A. The final product was purified by prep-HPLC under the following conditions: column, XBridge Shield RP18 OBD Column, 150×19 mm, 5 um; mobile phase, acetonitrile in water (with 10 mmol/L NH₄HCO₃ and 0.1% NH₃.H₂O), 30% to 55% gradient in 8 min; detector, UV 254 nm. 2-[[3,3-difluoro-1-(2-hydroxyacetyl)piperidin-4-yl]oxy]-5-(2-[[6-methoxy-5-(4-methylpiperazin-1-yl)pyridin-2-yl]amino]pyrimidin-4-yl)benzonitrile was obtained as a light yellow solid (226 mg, 22% for 2 steps). HPLC: 99.0% purity, RT=6.90 min. MS: m/z=595.0 [M+H]⁺. ¹H NMR (300 MHz, DMSO-d₆) δ 9.37 (s, 1H), 8.64-8.47 (m, 3H), 7.80-7.61 (m, 2H), 7.58-7.49 (m, 1H), 7.31-7.21 (m, 1H), 5.43-5.32 (m, 1H), 4.91-4.84 (m, 1H), 4.25-3.40 (m, 9H), 2.98-2.91 (m, 4H), 2.48-2.42 (m, 4H), 2.30-1.74 (m, 5H).

The title compounds were obtained by separation on chiral prep-HPLC under the following conditions: column, CHIRALPAK ID-3, 0.46×10 cm, 3 um; mobile phase, MtBE (with 0.1% DEA) in EtOH, 92% isocratic in 30 min; detector, UV 254 nm.

Example 100: 2-[[(4S)-3,3-difluoro-1-(2-hydroxyacetyl)piperidin-4-yl]oxy]-5-(2-[[6-methoxy-5-(4-methylpiperazin-1-yl)pyridin-2-yl]amino]pyrimidin-4-yl)benzonitrile 100: (73 mg, 37%, yellow solid) HPLC: 97.8% purity, RT=3.76 min. MS: m/z=595.0 [M+H]⁺. ¹H NMR (300 MHz, DMSO-d₆) δ 9.37 (s, 1H), 8.65-8.47 (m, 3H), 7.79-7.48 (m, 3H), 7.31-7.21 (m, 1H), 5.45-5.31 (m, 1H), 4.93-4.85 (m, 1H), 4.37-3.39 (m, 9H), 3.00-2.92 (m, 4H), 2.50-2.42 (m, 4H), 2.30-1.78 (m, 5H).

Example 101: 2-[[(4R)-3,3-difluoro-1-(2-hydroxyacetyl)piperidin-4-yl]oxy]-5-(2-[[6-methoxy-5-(4-methylpiperazin-1-yl)pyridin-2-yl]amino]pyrimidin-4-yl)benzonitrile 101: (67 mg, 34%, yellow solid) HPLC: 97.6% purity, RT=9.74 min. MS: m/z=595.0 [M+H]⁺. ¹H NMR (300 MHz, DMSO-d₆) δ 9.38 (s, 2H), 8.65-8.47 (m, 6H), 7.78-7.62 (m, 4H), 7.54 (d, J=5.2 Hz, 2H), 7.26 (d, J=8.3 Hz, 2H), 5.45-5.31 (m, 1H), 4.89 (br s, 1H), 4.28-3.41 (m, 9H), 2.98-2.92 (m, 7H), 2.49-2.42 (m, 7H), 2.22 (s, 5H).

### Example 102.2-[3,3-Difluoro-1-((R)-2-hydroxy-propionyl)-piperidin-4-yloxy]-5-{2-[5-((S)-3,4-dimethyl-piperazin-1-yl)-6-methoxy-pyridin-2-ylamino]-pyrimidin-4-yl}-benzonitrile 102

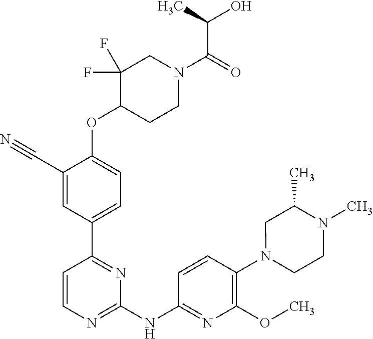 [SMILES: COc1nc(Nc2nccc(-c3ccc(OC4CCN(C(=O)[C@@H](C)O)CC4(F)F)c(C#N)c3)n2)ccc1N1CCN(C)[C@@H](C)C1]

The title compound (177 mg) was synthesized from 2-(3,3-Difluoro-piperidin-4-yloxy)-5-{2-[5-((S)-3,4-dimethyl-piperazin-1-yl)-6-methoxy-pyridin-2-ylamino]-pyrimidin-4-yl}-benzonitrile hydrochloride (127 mg), (R)-2-Hydroxy-propionic acid (19.48 mg), O-(7-Azabenzotriazol-1-yl)-N,N,N′,N′-tetramethyluronium hexaflurophosphate (HATU) (98 mg) and Ethyl-diisopropyl-amine (0.11 mL) using method A in 65% yield, m/z: 623 (M+H). ¹H NMR (DMSO-d6): 9.38 (s, 1H), 8.62-8.58 (m, 2H), 8.53 (dd, J=9.0, 2.3 Hz, 1H), 7.74 (d, J=8.2 Hz, 1H), 7.67 (d, J=9.2 Hz, 1H), 7.54 (d, J=5.2 Hz, 1H), 7.27 (d, J=8.3 Hz, 1H), 5.49-5.33 (m, 1H), 5.25-5.08 (m, 2H), 4.97 (p, J=6.7 Hz, 0H), 4.51 (d, J=9.3 Hz, 1H), 4.21 (d, J=6.6 Hz, 1H), 3.91 (s, 3H), 2.90 (d, J=11.3 Hz, 1H), 2.83-2.60 (m, 1H), 2.66-2.39 (m, 7H), 2.33 (s, 3H), 2.12 (d, J=37.0 Hz, 1H), 1.56-1.19 (m, 3H), 1.26 (dd, J=22.8, 6.7 Hz, 5H), 1.07 (d, J=5.3 Hz, 3H).

### Example 103: 2-[3,3-Difluoro-1-((R)-2-hydroxy-propionyl)-piperidin-4-yloxy]-5-{2-[5-((R)-3,4-dimethyl-piperazin-1-yl)-6-methoxy-pyridin-2-ylamino]-pyrimidin-4-yl}-benzonitrile 103

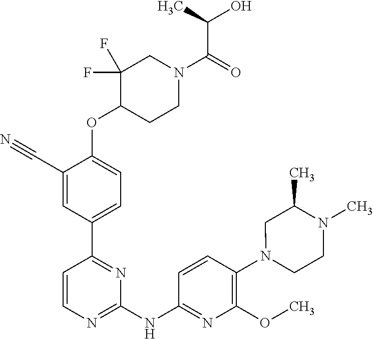 [SMILES: COc1nc(Nc2nccc(-c3ccc(OC4CCN(C(=O)[C@@H](C)O)CC4(F)F)c(C#N)c3)n2)ccc1N1CCN(C)[C@H](C)C1]

The title compound (24.5 mg) was synthesized from 2-(3,3-Difluoro-piperidin-4-yloxy)-5-{2-[5-((R)-3,4-dimethyl-piperazin-1-yl)-6-methoxy-pyridin-2-ylamino]-pyrimidin-4-yl}-benzonitrile hydrochloride (86 mg), (R)-2-Hydroxy-propionic acid (13 mg), O-(7-Azabenzotriazol-1-yl)-N,N,N′,N′-tetramethyluronium hexaflurophosphate (HATU) (66 mg) and Ethyl-diisopropyl-amine (56 mg) using Method A in 27% yield, m/z: 623 (M+H). ¹H NMR (DMSO-d6): 9.35 (s, 1H), 8.63-8.49 (m, 3H), 8.28 (s, 1H), 7.70 (dd, J=25.9, 8.6 Hz, 2H), 7.54 (d, J=5.2 Hz, 1H), 7.25 (d, J=8.3 Hz, 1H), 5.38 (dd, J=13.4, 7.3 Hz, 1H), 4.51 (q, J=6.5 Hz, 1H), 3.91 (s, 4H), 3.21 (dd, J=27.3, 10.9 Hz, 2H), 2.84-2.59 (m, 2H), 2.51 (p, J=1.8 Hz, 2H), 2.22 (s, 3H), 1.23 (d, J=6.4 Hz, 3H), 1.02 (d, J=6.1 Hz, 3H).

### Example 104: 2-[3,3-Difluoro-1-((R)-2-hydroxy-propionyl)-piperidin-4-yloxy]-5-{2-[6-methoxy-5-(4-methyl-piperazin-1-yl)-pyridin-2-ylamino]-pyrimidin-4-yl}-benzonitrile 104

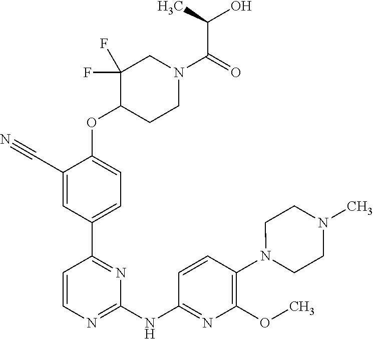 [SMILES: COc1nc(Nc2nccc(-c3ccc(OC4CCN(C(=O)[C@@H](C)O)CC4(F)F)c(C#N)c3)n2)ccc1N1CCN(C)CC1]

The title compound (24.3 mg) was synthesized from 2-(3,3-Difluoro-piperidin-4-yloxy)-5-{2-[6-methoxy-5-(4-methyl-piperazin-1-yl)-pyridin-2-ylamino]-pyrimidin-4-yl}-benzonitrile hydrochloride (80 mg), (R)-2-Hydroxy-propionic acid (13 mg), O-(7-Azabenzotriazol-1-yl)-N,N,N′,N′-tetramethyluronium hexaflurophosphate (HATU) (63 mg) and Ethyl-diisopropyl-amine (54 mg) using Method A in 29% yield, m/z: 609 (M+H). ¹H NMR (DMSO-d6): 9.35 (d, J=2.2 Hz, 1H), 8.63-8.57 (m, 2H), 8.53 (dd, J=9.0, 2.2 Hz, 1H), 7.73 (d, J=8.3 Hz, 1H), 7.67 (dd, J=9.3, 1.9 Hz, 1H), 7.54 (d, J=5.2 Hz, 1H), 7.26 (d, J=8.3 Hz, 1H), 5.37 (d, J=13.7 Hz, 1H), 5.22 (s, 1H), 4.51 (s, 1H), 3.91 (s, 3H), 2.96 (s, 4H), 2.52-2.43 (m, 6H), 2.24 (s, 3H), 1.23 (dd, J=6.7, 3.3 Hz, 4H).

### Example 105.2-[3,3-Difluoro-1-((S)-2-hydroxy-propionyl)-piperidin-4-yloxy]-5-{2-[5-((S)-3,4-dimethyl-piperazin-1-yl)-6-methoxy-pyridin-2-ylamino]-pyrimidin-4-yl}-benzonitrile 105

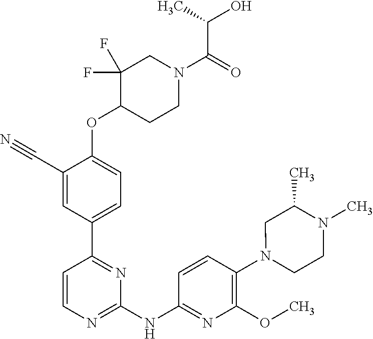 [SMILES: COc1nc(Nc2nccc(-c3ccc(OC4CCN(C(=O)[C@H](C)O)CC4(F)F)c(C#N)c3)n2)ccc1N1CCN(C)[C@@H](C)C1]

The title compound (66 mg) was synthesized from 2-(3,3-Difluoro-piperidin-4-yloxy)-5-{2-[5-((S)-3,4-dimethyl-piperazin-1-yl)-6-methoxy-pyridin-2-ylamino]-pyrimidin-4-yl}-benzonitrile hydrochloride (127 mg), (S)-2-Hydroxy-propionic acid (19.48 mg), O-(7-Azabenzotriazol-1-yl)-N,N,N′,N′-tetramethyluronium hexaflurophosphate (HATU) (98 mg) and Ethyl-diisopropyl-amine (0.11 mL) using Method A in 44% yield, m/z: 623 (M+H). ¹H NMR (DMSO-d6): 9.38 (s, 1H), 8.62-8.58 (m, 2H), 8.53 (dd, J=9.0, 2.3 Hz, 1H), 7.74 (d, J=8.2 Hz, 1H), 7.67 (d, J=9.2 Hz, 1H), 7.54 (d, J=5.2 Hz, 1H), 7.27 (d, J=8.3 Hz, 1H), 5.49-5.33 (m, 1H), 5.25-5.08 (m, 2H), 4.97 (p, J=6.7 Hz, 0H), 4.51 (d, J=9.3 Hz, 1H), 4.21 (d, J=6.6 Hz, 1H), 3.91 (s, 3H), 2.90 (d, J=11.3 Hz, 1H), 2.83-2.60 (m, 1H), 2.66-2.39 (m, 7H), 2.33 (s, 3H), 2.12 (d, J=37.0 Hz, 1H), 1.56-1.19 (m, 3H), 1.26 (dd, J=22.8, 6.7 Hz, 5H), 1.07 (d, J=5.3 Hz, 3H).

### Example 106: 2-[3,3-Difluoro-1-((S)-2-hydroxy-propionyl)-piperidin-4-yloxy]-5-{2-[5-((R)-3,4-dimethyl-piperazin-1-yl)-6-methoxy-pyridin-2-ylamino]-pyrimidin-4-yl}-benzonitrile 106

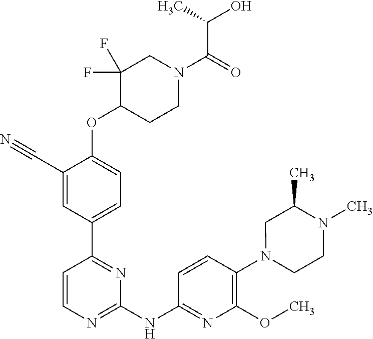 [SMILES: COc1nc(Nc2nccc(-c3ccc(OC4CCN(C(=O)[C@H](C)O)CC4(F)F)c(C#N)c3)n2)ccc1N1CCN(C)[C@H](C)C1]

The title compound (19.3 mg) was synthesized from 2-(3,3-Difluoro-piperidin-4-yloxy)-5-{2-[5-((R)-3,4-dimethyl-piperazin-1-yl)-6-methoxy-pyridin-2-ylamino]-pyrimidin-4-yl}-benzonitrile hydrochloride (86 mg), (S)-2-Hydroxy-propionic acid (13 mg), O-(7-Azabenzotriazol-1-yl)-N,N,N′,N′-tetramethyluronium hexaflurophosphate (HATU) (66 mg) and Ethyl-diisopropyl-amine (56 mg) using Method A in 21% yield, m/z: 623 (M+H). ¹H NMR (DMSO-d6): 9.35 (s, 1H), 8.63-8.49 (m, 3H), 8.28 (s, 1H), 7.70 (dd, J=25.9, 8.6 Hz, 2H), 7.54 (d, J=5.2 Hz, 1H), 7.25 (d, J=8.3 Hz, 1H), 5.38 (dd, J=13.4, 7.3 Hz, 1H), 4.51 (q, J=6.5 Hz, 1H), 3.91 (s, 4H), 3.21 (dd, J=27.3, 10.9 Hz, 2H), 2.84-2.59 (m, 2H), 2.51 (p, J=1.8 Hz, 2H), 2.22 (s, 3H), 1.23 (d, J=6.4 Hz, 3H), 1.02 (d, J=6.1 Hz, 3H).

### Example 107: 2-[3,3-Difluoro-1-((S)-2-hydroxy-propionyl)-piperidin-4-yloxy]-5-{2-[6-methoxy-5-(4-methyl-piperazin-1-yl)-pyridin-2-ylamino]-pyrimidin-4-yl}-benzonitrile 107

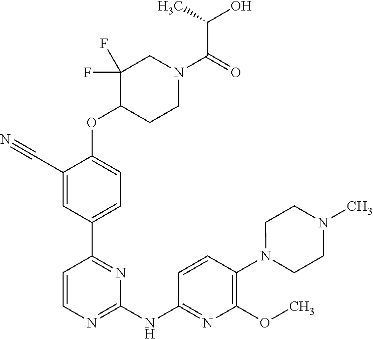 [SMILES: COc1nc(Nc2nccc(-c3ccc(OC4CCN(C(=O)[C@H](C)O)CC4(F)F)c(C#N)c3)n2)ccc1N1CCN(C)CC1]

The title compound (19.6 mg) was synthesized according to the procedure described to example 9 using 2-(3,3-Difluoro-piperidin-4-yloxy)-5-{2-[6-methoxy-5-(4-methyl-piperazin-1-yl)-pyridin-2-ylamino]-pyrimidin-4-yl}-benzonitrile (78.5 mg, 0.15 mmol), (S)-2-Hydroxy-propionic acid (14.28 mg; 0.24 mmol; 2.00 eq.), O-(7-Azabenzotriazol-1-yl)-N,N,N′,N′-tetramethyluronium hexaflurophosphate (HATU) (66.70 mg; 0.21 mmol; 1.75 eq.) and Ethyl-diisopropyl-amine (0.08 mL) in 92% yield, m/z: 609 (M+H). ¹H NMR (DMSO-d6): 9.36 (s, 1H), 8.64-8.49 (m, 3H), 7.70 (dd, J=24.4, 8.8 Hz, 2H), 7.54 (d, J=5.3 Hz, 1H), 7.27 (d, J=8.3 Hz, 1H), 5.42-5.34 (m, 1H), 5.22 (d, J=6.8 Hz, 1H), 4.51 (s, 1H), 4.17 (s, 1H), 3.91 (s, 4H), 3.65 (s, 1H), 2.96 (s, 4H), 2.46 (s, 4H), 2.23 (s, 3H), 1.26-1.13 (m, 4H).

### Example 108: 2-(3,3-Difluoro-piperidin-4-yloxy)-5-{2-[5-((R)-2,4-dimethyl-piperazin-1-yl)-6-methoxy-pyridin-2-ylamino]-pyrimidin-4-yl}-benzonitrile 108

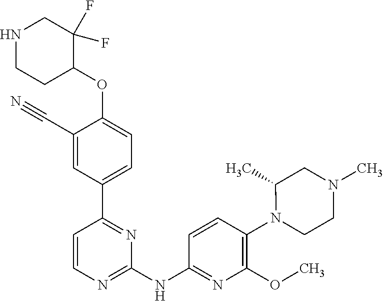 [SMILES: COc1nc(Nc2nccc(-c3ccc(OC4CCNCC4(F)F)c(C#N)c3)n2)ccc1N1CCN(C)C[C@H]1C]

### (R)-1-(6-Bromo-2-methoxy-pyridin-3-yl)-2,4-dimethyl-piperazine

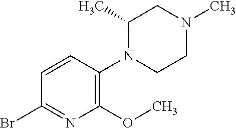 [SMILES: COc1nc(Br)ccc1N1CCN(C)C[C@H]1C]

To a solution of (R)-1-(2-Methoxy-pyridin-3-yl)-2,4-dimethyl-piperazine (4600.00 mg; 20.79 mmol; 1.00 eq.) in DMF (30 mL), cooled to −50° C., then 1-Bromo-pyrrolidine-2,5-dione (4439.57 mg; 24.94 mmol; 1.20 eq.) in DMF (10 mL) was added dropwise. The resulting solution was stirred at this temperature for 2 hours. 200 mL of water was added and the cooling bath was removed. The solution was neutralized to pH to 8-9 with addition of aqueous potassium carbonate and the mixture was extracted with EtOAc (3×200 mL). The combined organic layer was dried over MgSO₄, filtrated and concentrated. The crude product was purified through flash chromatography on silica gel (Hex/EtOAc from 0% to 100% containing 1% triethylamine) to provide the desired product (S)-1-(6-Bromo-2-methoxy-pyridin-3-yl)-2,4-dimethyl-piperazine (4150.00 mg; 13.82 mmol) in 66% yield, m/z 301 (M+H)

### 4-(2-Cyano-4-{2-[5-((R)-2,4-dimethyl-piperazin-1-yl)-6-methoxy-pyridin-2-ylamino]-pyrimidin-4-yl}-phenoxy)-3,3-difluoro-piperidine-1-carboxylic acid tert-butyl ester

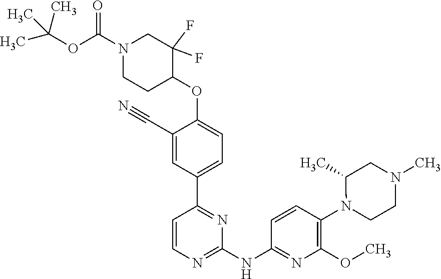 [SMILES: COc1nc(Nc2nccc(-c3ccc(OC4CCN(C(=O)OC(C)(C)C)CC4(F)F)c(C#N)c3)n2)ccc1N1CCN(C)C[C@H]1C]

A mixture of 4-[4-(2-Amino-pyrimidin-4-yl)-2-cyano-phenoxy]-3,3-difluoro-piperidine-1-carboxylic acid tert-butyl ester (200.00 mg; 0.46 mmol; 1.00 eq.), (R)-1-(6-Bromo-2-methoxy-pyridin-3-yl)-2,4-dimethyl-piperazine (417.48 mg; 1.39 mmol; 3.00 eq.), 4,5-Bis-diphenylphosphanyl-9,9-dimethyl-9H-xanthene (90 mg, 0.14 mmol; 0.30 eq.), and Cs₂CO₃ (476.97 mg; 1.39 mmol; 3.00 eq.) in Dioxane (10 mL) in a microwave vial was purged with argon for 3 minutes. Then Pd₂(dba)₃CHCl₃ (101.02 mg; 0.09 mmol; 0.20 eq.) was added. The reaction mixture was heated at 100° C. for 5 hours. The reaction mixture was filtered, and concentrated. The crude was dissolved in DMF (4 mL) and loaded on reverse phase HPLC to provide 4-(2-Cyano-4-{2-[5-((S)-2,4-dimethyl-piperazin-1-yl)-6-methoxy-pyridin-2-ylamino]-pyrimidin-4-yl}-phenoxy)-3,3-difluoro-piperidine-1-carboxylic acid tert-butyl ester (200.00 mg; 0.18 mmol) in 62% yield, m/z: 651 (M+H)⁺.

### 2-(3,3-Difluoro-piperidin-4-yloxy)-5-{2-[5-((R)-2,4-dimethyl-piperazin-1-yl)-6-methoxy-pyridin-2-ylamino]-pyrimidin-4-yl}-benzonitrile

The title compound (500 mg) was synthesized according to the procedure described in Method 34 using 4-(2-Cyano-4-{2-[5-((R)-2,4-dimethyl-piperazin-1-yl)-6-methoxy-pyridin-2-ylamino]-pyrimidin-4-yl}-phenoxy)-3,3-difluoro-piperidine-1-carboxylic acid tert-butyl ester (1200 mg) and HCl in Dioxane (4M, 25 mL) in 47% yield, m/z: 551 (M+H). ¹H NMR (DMSO-d6): 9.41 (1H), 8.62 (2H), 8.51 (1H), 7.76 (1H), 7.67 (1H), 7.55 (1H), 7.34 (1H), 5.22 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 0.82 (3H).

### Example 109: 2-(3,3-Difluoro-piperidin-4-yloxy)-5-{2-[5-((S)-2,4-dimethyl-piperazin-1-yl)-6-methoxy-pyridin-2-ylamino]-pyrimidin-4-yl}-benzonitrile 109

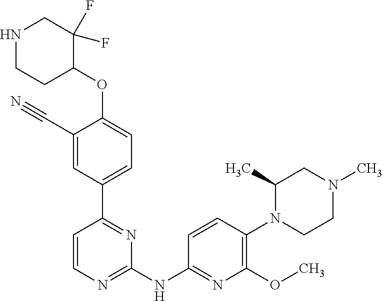 [SMILES: COc1nc(Nc2nccc(-c3ccc(OC4CCNCC4(F)F)c(C#N)c3)n2)ccc1N1CCN(C)C[C@@H]1C]

The title compound was prepared according to the procedure described in example 108 by coupling (S)-1-(2-Methoxy-pyridin-3-yl)-2,4-dimethyl-piperazine and 4-[4-(2-Amino-pyrimidin-4-yl)-2-cyano-phenoxy]-3,3-difluoro-piperidine-1-carboxylic acid tert-butyl ester to get 4-(2-Cyano-4-{2-[5-((S)-2,4-dimethyl-piperazin-1-yl)-6-methoxy-pyridin-2-ylamino]-pyrimidin-4-yl}-phenoxy)-3,3-difluoro-piperidine-1-carboxylic acid tert-butyl ester followed by treatment with HCl (4M in Dioxane). m/z: 551 (M+H). ¹H NMR (DMSO-d6): 9.41 (1H), 8.62 (2H), 8.51 (1H), 7.76 (1H), 7.67 (1H), 7.55 (1H), 7.34 (1H), 5.22 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 0.82 (3H).

### Example 110:2-[3,3-Difluoro-1-((R)-2-hydroxy-propionyl)-piperidin-4-yloxy]-5-{2-[5-((S)-2,4-dimethyl-piperazin-1-yl)-6-methoxy-pyridin-2-ylamino]-pyrimidin-4-yl}-benzonitrile

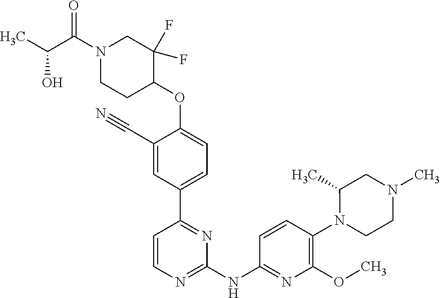 [SMILES: COc1nc(Nc2nccc(-c3ccc(OC4CCN(C(=O)[C@@H](C)O)CC4(F)F)c(C#N)c3)n2)ccc1N1CCN(C)C[C@H]1C]

The title compound (56 mg) was synthesized using 2-(3,3-Difluoro-piperidin-4-yloxy)-5-{2-[5-((R)-2,4-dimethyl-piperazin-1-yl)-6-methoxy-pyridin-2-ylamino]-pyrimidin-4-yl}-benzonitrile (80 mg), DIPEA (94 mg), HATU (97 mg) and (R)-2-Hydroxy-propionic acid (26.18 mg) using Method A in 59% yield, m/z: 623 (M+H). ¹H NMR (DMSO-d6): 9.41 (1H), 8.62 (2H), 8.51 (1H), 7.76 (1H), 7.67 (1H), 7.55 (1H), 7.34 (1H), 5.38 (1H), 5.22 (1H), 4.53 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 1.25 93H), 0.82 (3H).

### Example 111: 2-[3,3-Difluoro-1-((R)-2-hydroxy-propionyl)-piperidin-4-yloxy]-5-{2-[5-((S)-2,4-dimethyl-piperazin-1-yl)-6-methoxy-pyridin-2-ylamino]-pyrimidin-4-yl}-benzonitrile 111

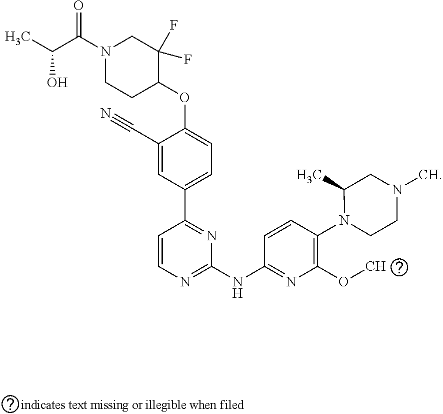

The title compound was prepared by using 2-(3,3-Difluoro-piperidin-4-yloxy)-5-{2-[5-((S)-2,4-dimethyl-piperazin-1-yl)-6-methoxy-pyridin-2-ylamino]-pyrimidin-4-yl}-benzonitrile (80.00 mg; 0.15 mmol; 1.00 eq.), (R)-2-Hydroxy-propionic acid (26.18 mg; 0.29 mmol; 2.00 eq.), O-(7-Azabenzotriazol-1-yl)-N,N,N′,N′-tetramethyluronium hexaflurophosphate (HATU) (96.94 mg; 0.25 mmol; 1.75 eq.) and Ethyl-diisopropyl-amine (93.89 mg; 0.73 mmol; 5.00 eq.) in DMF (3 mL). The crude was purified on reverse phase HPLC to provide 2-[3,3-Difluoro-1-((R)-2-hydroxy-propionyl)-piperidin-4-yloxy]-5-{2-[5-((S)-2,4-dimethyl-piperazin-1-yl)-6-methoxy-pyridin-2-ylamino]-pyrimidin-4-yl}-benzonitrile (25.90 mg; 0.04 mmol) using Method A in 28% yield, m/z: 623 (M+H). ¹H NMR (DMSO-d6): 9.41 (1H), 8.62 (2H), 8.51 (1H), 7.76 (1H), 7.67 (1H), 7.55 (1H), 7.34 (1H), 5.38 (1H), 5.22 (1H), 4.53 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 1.25 93H), 0.82 (3H).

### Example 112: 2-[3,3-Difluoro-1-((S)-2-hydroxy-propionyl)-piperidin-4-yloxy]-5-{2-[5-((S)-2,4-dimethyl-piperazin-1-yl)-6-methoxy-pyridin-2-ylamino]-pyrimidin-4-yl}-benzonitrile 112

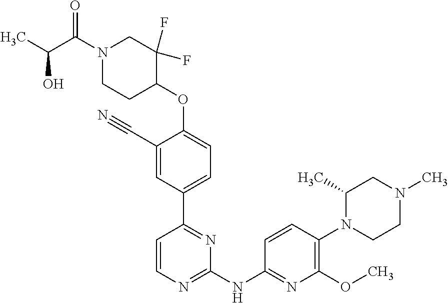 [SMILES: COc1nc(Nc2nccc(-c3ccc(OC4CCN(C(=O)[C@H](C)O)CC4(F)F)c(C#N)c3)n2)ccc1N1CCN(C)C[C@H]1C]

The title compound (90 mg) was synthesized using 2-(3,3-Difluoro-piperidin-4-yloxy)-5-{2-[5-((R)-2,4-dimethyl-piperazin-1-yl)-6-methoxy-pyridin-2-ylamino]-pyrimidin-4-yl}-benzonitrile (80 mg), DIPEA (94 mg), HATU (97 mg) and (S)-2-Hydroxy-propionic acid (26.18 mg) using Method A in 91% yield, m/z: 623 (M+H). ¹H NMR (DMSO-d6): 9.41 (1H), 8.62 (2H), 8.51 (1H), 7.76 (1H), 7.67 (1H), 7.55 (1H), 7.34 (1H), 5.38 (1H), 5.22 (1H), 4.53 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 1.25 93H), 0.82 (3H).

### Example 113: 2-[3,3-Difluoro-1-((S)-2-hydroxy-propionyl)-piperidin-4-yloxy]-5-{2-[5-((S)-2,4-dimethyl-piperazin-1-yl)-6-methoxy-pyridin-2-ylamino]-pyrimidin-4-yl}-benzonitrile

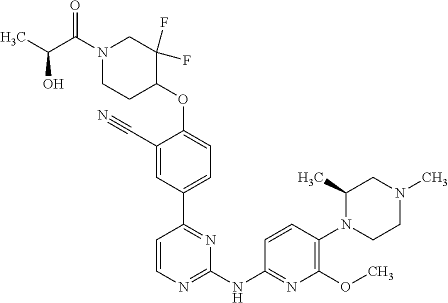 [SMILES: COc1nc(Nc2nccc(-c3ccc(OC4CCN(C(=O)[C@H](C)O)CC4(F)F)c(C#N)c3)n2)ccc1N1CCN(C)C[C@@H]1C]

The title compound (40 mg) was synthesized from 2-(3,3-Difluoro-piperidin-4-yloxy)-5-{2-[5-((S)-2,4-dimethyl-piperazin-1-yl)-6-methoxy-pyridin-2-ylamino]-pyrimidin-4-yl}-benzonitrile (80 mg), DIPEA (94 mg), HUTA (97 mg) and (R)-2-Hydroxy-propionic acid (26.18 mg) using Method A in 45% yield, m/z: 623 (M+H). ¹H NMR (DMSO-d6): 9.41 (1H), 8.62 (2H), 8.51 (1H), 7.76 (1H), 7.67 (1H), 7.55 (1H), 7.34 (1H), 5.38 (1H), 5.22 (1H), 4.53 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 1.25 93H), 0.82 (3H).

### Example 114: 2-[3,3-Difluoro-1-(2-hydroxy-2-methyl-propionyl)-piperidin-4-yloxy]-5-{2-[5-((R)-2,4-dimethyl-piperazin-1-yl)-6-methoxy-pyridin-2-ylamino]-pyrimidin-4-yl}-benzonitrile 114

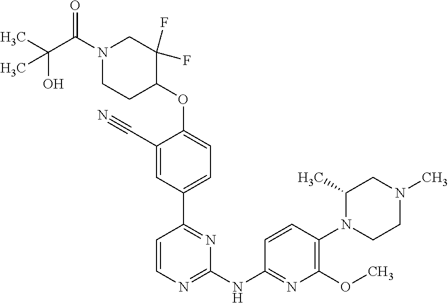 [SMILES: COc1nc(Nc2nccc(-c3ccc(OC4CCN(C(=O)C(C)(C)O)CC4(F)F)c(C#N)c3)n2)ccc1N1CCN(C)C[C@H]1C]

The title compound (17.6 mg) was synthesized from 2-(3,3-Difluoro-piperidin-4-yloxy)-5-{2-[5-((R)-2,4-dimethyl-piperazin-1-yl)-6-methoxy-pyridin-2-ylamino]-pyrimidin-4-yl}-benzonitrile (80 mg), DIPEA (94 mg), HUTA (97 mg) and 2-Hydroxy-2-methyl-propionic acid (30.25 mg; 0.29 mmol; 2.00 eq.) using Method A in 18% yield, m/z: 637 (M+H). ¹H NMR (DMSO-d6): 9.43 (1H), 8.62 (2H), 8.53 (1H), 7.76 (1H), 7.65 (1H), 7.55 (1H), 7.34 (1H), 5.65 (1H), 5.38 (1H), 5.22 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 1.37 (6H), 0.82 (3H).

### Example 115: 2-[[(3R,4S)-3-fluoro-1-[(1H-1,2,3-triazol-5-yl)carbonyl]piperidin-4-yl]oxy]-5 [2-([6-methoxy-5-[4-(oxetan-3-yl)piperazin-1-yl]pyridin-2-yl]amino)pyrimidin-4-yl]benzonitrile 115

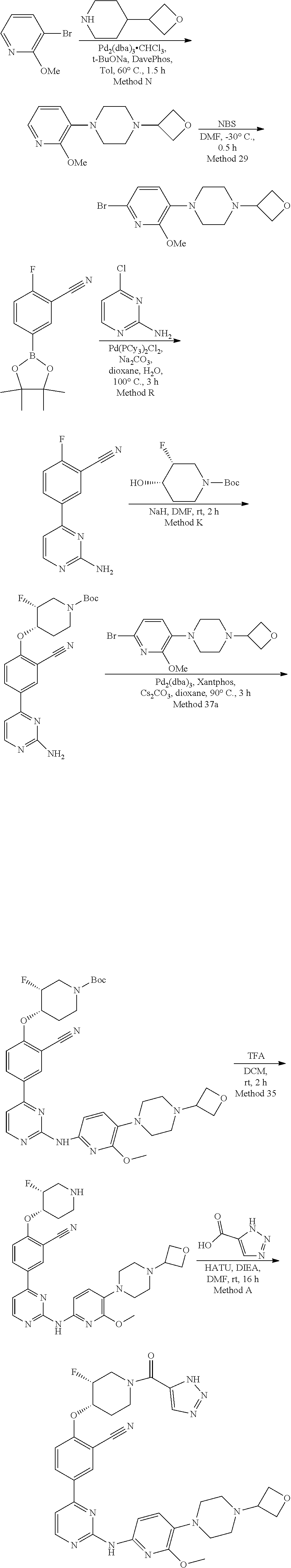

### Method N

1-(2-methoxypyridin-3-yl)-4-(oxetan-3-yl)piperazine: To a solution of 3-bromo-2-methoxypyridine (1.80 g, 9.59 mmol) in toluene (50 mL) was added 1-(oxetan-3-yl)piperazine (1.85 g, 13.06 mmol), Pd₂(dba)₃CHCl₃ (479 mg, 0.46 mmol), DavePhos (578 mg, 1.47 mmol) and t-BuONa (1.41 g, 14.64 mmol) at room temperature. The resulting mixture was stirred for 1.5 h at 60° C. When the reaction was done, the reaction mixture was concentrated under reduced pressure and the residue was purified by flash chromatography eluting with EtOAc in hexane (0% to 70% gradient) to yield 1-(2-methoxypyridin-3-yl)-4-(oxetan-3-yl)piperazine as brown oil (1.28 g, 54%). MS: m/z=250.1 [M+H]⁺.

1-(6-bromo-2-methoxypyridin-3-yl)-4-(oxetan-3-yl)piperazine: 1-(6-bromo-2-methoxypyridin-3-yl)-4-(oxetan-3-yl)piperazine was prepared from 1-(2-methoxypyridin-3-yl)-4-(oxetan-3-yl)piperazine and NBS using Method 29. The final product was purified by flash chromatography eluting with EtOAc in hexane (0% to 70% gradient) to yield 1-(6-bromo-2-methoxypyridin-3-yl)-4-(oxetan-3-yl)piperazine as a yellow solid (1.46 g, 86%). MS: m/z=327.9 [M+H]⁺.

### Method R

1-(2-methoxypyridin-3-yl)-4-(oxetan-3-yl)piperazine: To a solution of 2-fluoro-5-(tetramethyl-1,3,2-dioxaborolan-2-yl)benzonitrile (1.71 g, 6.92 mmol) in dioxane (40 mL) was added 4-chloropyrimidin-2-amine (950 mg, 7.33 mmol), sodium carbonate solution (1.4 M in water, 10 mL, 14.00 mmol) and Pd(PCy)₂Cl₂ (1.08 g, 1.47 mmol) at room temperature. The resulting mixture was stirred for 3 h at 100° C. When the reaction was done, the reaction mixture was concentrated under reduced pressure and the residue was purified by flash chromatography eluting with EtOAc in hexane (0% to 100% gradient) to yield 5-(2-aminopyrimidin-4-yl)-2-fluorobenzonitrile as an yellow solid (990 mg, 66%). MS: m/z=215.0 [M+H]⁺.

tert-butyl (3R,4S)-4-[4-(2-aminopyrimidin-4-yl)-2-cyanophenoxy]-3-fluoropiperidine-1-carboxylate: tert-butyl (3R,4S)-4-[4-(2-aminopyrimidin-4-yl)-2-cyanophenoxy]-3-fluoropiperidine-1-carboxylate was prepared from tert-butyl (3R,4S)-3-fluoro-4-hydroxypiperidine-1-carboxylate and 5-(2-aminopyrimidin-4-yl)-2-fluorobenzonitrile using Method K to yield tert-butyl (3R,4S)-4-[4-(2-aminopyrimidin-4-yl)-2-cyanophenoxy]-3-fluoropiperidine-1-carboxylate as brown oil (484 mg, 94%). MS: m/z=414.4 [M+H]⁺.

### Method 37a

tert-butyl (3R,4S)-4-[2-cyano-4-[2-([6-methoxy-5-[4-(oxetan-3-yl)piperazin-1-yl]pyridin-2-yl]amino)pyrimidin-4-yl]phenoxy]-3-fluoropiperidine-1-carboxylate: To a solution of tert-butyl (3R,4S)-4-[4-(2-aminopyrimidin-4-yl)-2-cyanophenoxy]-3-fluoropiperidine-1-carboxylate (504 mg, 1.22 mmol) in 1,4-dioxane (30 mL) were added 1-(6-bromo-2-methoxypyridin-3-yl)-4-(oxetan-3-yl)piperazine (866 mg, 2.64 mmol), Pd₂(dba)₃CHCl₃ (133 mg, 0.13 mmol), Xantphos (149 mg, 0.26 mmol) and Cs₂CO₃ (851 mg, 2.61 mmol) at room temperature. The resulting mixture was stirred for 3 h at 90° C. When the reaction was done, the reaction mixture was concentrated under reduced pressure and the residue was purified by flash chromatography eluting with EtOAc in hexane (0% to 100% gradient) to yield tert-butyl (3R,4S)-4-[2-cyano-4-[2-([6-methoxy-5-[4-(oxetan-3-yl)piperazin-1-yl]pyridin-2-yl]amino)pyrimidin-4-yl]phenoxy]-3-fluoropiperidine-1-carboxylate as an yellow solid (173 mg, 21%). MS: m/z=661.3 [M+H]⁺.

2-[[(3R,4S)-3-fluoro-1-[(1H-1,2,3-triazol-5-yl)carbonyl]piperidin-4-yl]oxy]-5-[2-([6-methoxy-5-[4-(oxetan-3-yl)piperazin-1-yl]pyridin-2-yl]amino)pyrimidin-4-yl]benzonitrile: 2-[[(3R,4S)-3-fluoro-1-[(1H-1,2,3-triazol-5-yl)carbonyl]piperidin-4-yl]oxy]-5-[2-([6-methoxy-5-[4-(oxetan-3-yl)piperazin-1-yl]pyridin-2-yl]amino)pyrimidin-4-yl]benzonitrile was prepared from 2-[[(3R,4S)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile and 1H-1,2,3-triazole-5-carboxylic acid using Method 35 and A. The final product was purified by prep-HPLC under the following conditions: column, XBridge Shield RP18 OBD Column, 150×19 mm, 5 um; mobile phase, acetonitrile in water (with 10 mmol/L NH₄HCO₃ and 0.1% NH₃.H₂O), 25% to 34% gradient in 7 min; detector, UV 254 nm. 2-[[(3R,4S)-3-fluoro-1-[(1H-1,2,3-triazol-5-yl)carbonyl]piperidin-4-yl]oxy]-5-[2-([6-methoxy-5-[4-(oxetan-3-yl)piperazin-1-yl]pyridin-2-yl]amino)pyrimidin-4-yl]benzonitrile was obtained as an yellow solid (30 mg, 21% for 2 steps). HPLC: 98.4% purity, RT=2.28 min. MS: m/z=656.6 [M+H]⁺. ¹H NMR (300 MHz, DMSO-d₆) δ 9.38 (s, 1H), 8.67-8.45 (m, 3H), 8.24 (s, 1H), 7.81-7.47 (m, 3H), 7.32-7.23 (m, 1H), 5.38-4.91 (m, 2.5H), 4.70-4.17 (m, 5.5H), 4.04-3.55 (m, 5H), 3.54-3.42 (m, 1H), 3.02-2.95 (m, 4H), 2.44-2.38 (m, 4H), 2.15-1.88 (m, 2H).

### Example 116: 2-[(3R,4S)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

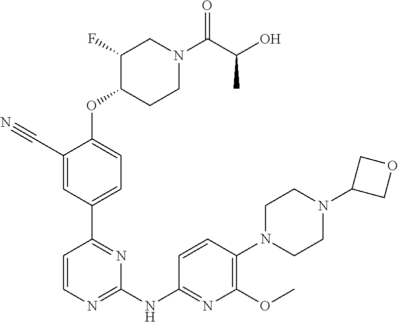 [SMILES: COc1nc(Nc2nccc(-c3ccc(O[C@H]4CCN(C(=O)[C@H](C)O)C[C@H]4F)c(C#N)c3)n2)ccc1N1CCN(C2COC2)CC1]

(3R,4S)-4-[4-(2-Amino-pyrimidin-4-yl)-2-cyano-phenoxy]-3-fluoro-piperidine-1-carboxylic acid tert-butyl ester To a mixture of 4-Chloro-pyrimidin-2-ylamine (0.50 g; 3.86 mmol; 1.00 eq.), (3R,4S)-4-[2-Cyano-4-(4,4,5,5-tetramethyl-[1,3,2]dioxaborolan-2-yl)-phenoxy]-3-fluoro-piperidine-1-carboxylic acid tert-butyl ester (2.07 g; 4.63 mmol; 1.20 eq.), and potassium phosphate (1.64 g; 7.72 mmol; 2.00 eq.) in a pressure bottle were added N,N-Dimethyl-formamide (15.00 ml) and water (3.00 ml). The reaction mixture was sparged with Argon for 15 min. cyclopentyl(diphenyl)phosphane; dichloropalladium; iron (0.56 g; 0.77 mmol; 0.20 eq.) (Pd(dppf) was added. The reaction mixture was heated at 110° C. overnight using an oil bath. Filtered and washed with methanol. The solvent was removed and the crude was purified on Intechim 120 g column with ethyl acetate-methanol to obtain (3R,4S)-4-[4-(2-Amino-pyrimidin-4-yl)-2-cyano-phenoxy]-3-fluoro-piperidine-1-carboxylic acid tert-butyl ester (1.03 g; 64.5%). ¹H NMR (400 MHz, Chloroform-d) δ 8.38 (d, J=5.1 Hz, 1H), 8.34-8.26 (m, 1H), 8.19 (dd, J=8.9, 2.3 Hz, 1H), 7.16 (d, J=8.9 Hz, 1H), 6.98 (d, J=5.2 Hz, 1H), 5.15 (s, 2H), 4.92 (d, J=5.2 Hz, 1H), 4.76 (d, J=46.0 Hz, 1H), 3.96 (s, 1H), 3.70 (d, J=14.2 Hz, 2H), 3.53 (ddd, J=13.6, 9.8, 3.2 Hz, 1H), 2.94 (d, J=28.7 Hz, 1H), 2.15 (tt, J=9.8, 4.7 Hz, 1H), 1.50 (s, 8H). MS: m/z=414.2 [M+H]⁺.

(3R,4S)-4-(2-Cyano-4-{2-[6-methoxy-5-(4-oxetan-3-yl-piperazin-1-yl)-pyridin-2-ylamino]-pyrimidin-4-yl}-phenoxy)-3-fluoro-piperidine-1-carboxylic acid tert-butyl ester: A mixture of (3R,4S)-4-[4-(2-Amino-pyrimidin-4-yl)-2-cyano-phenoxy]-3-fluoro-piperidine-1-carboxylic acid tert-butyl ester (275.00 mg; 0.67 mmol; 1.00 eq.), 1-(6-Bromo-2-methoxy-pyridin-3-yl)-4-oxetan-3-yl-piperazine (218.30 mg; 0.67 mmol; 1.00 eq.), Xantphos (145.32 mg; 0.22 mmol; 0.33 eq.), and Cs₂CO₃ (456.24 mg; 1.33 mmol; 2.00 eq.) in N,N-Dimethyl-formamide (15.00 ml) in a microwave vial was purged with argon for 15 min. Then Pd₂(dba)₃CHCl₃ (79.72 mg; 0.07 mmol; 0.11 eq.) was added. The reaction mixture was heated at 100° C. for 1 h under microwave irradiation. Filtered, DMF was removed and ethyl acetate was added to the residue to get a solid. Filtered and washed with ethyl acetate to obtain (3R,4S)-4-(2-Cyano-4-{2-[6-methoxy-5-(4-oxetan-3-yl-piperazin-1-yl)-pyridin-2-ylamino]-pyrimidin-4-yl}-phenoxy)-3-fluoro-piperidine-1-carboxylic acid tert-butyl ester (330.00 mg, 65.3%) MS: m/z=661.3 [M+H]⁺.

2-((3R,4S)-3-Fluoro-piperidin-4-yloxy)-5-{2-[6-methoxy-5-(4-oxetan-3-yl-piperazin-1-yl)-pyridin-2-ylamino]-pyrimidin-4-yl}-benzonitrile hydrochloride: (3R,4S)-4-(2-Cyano-4-{2-[6-methoxy-5-(4-oxetan-3-yl-piperazin-1-yl)-pyridin-2-ylamino]-pyrimidin-4-yl}-phenoxy)-3-fluoro-piperidine-1-carboxylic acid tert-butyl ester (338.00 mg; 0.43 mmol; 1.00 eq.) (84% purity) was dissolved in Dichloromethane (50.00 ml). To this hydrogen chloride in dioxane (1.07 ml; 2.15 mmol; 5.00 eq.) was added. Stirred overnight at room temperature. The product 2-((3R,4S)-3-Fluoro-piperidin-4-yloxy)-5-{2-[6-methoxy-5-(4-oxetan-3-yl-piperazin-1-yl)-pyridin-2-ylamino]-pyrimidin-4-yl}-benzonitrile hydrochloride (245.00 mg; 95%) was precipitated as an yellow colored solid. MS: m/z=561.3 [M+H]⁺.

2-[(3R,4S)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile: A mixture of 2-((3R,4S)-3-Fluoro-piperidin-4-yloxy)-5-{2-[6-methoxy-5-(4-oxetan-3-yl-piperazin-1-yl)-pyridin-2-ylamino]-pyrimidin-4-yl}-benzonitrile hydrochloride (125.00 mg; 0.21 mmol; 1.00 eq.), [dimethylamino(triazolo[4,5-b]pyridin-3-yloxy)methylene]-dimethyl-ammonium hexafluorophosphate (HATU) (95.52 mg; 0.25 mmol; 1.20 eq.) and Ethyl-diisopropyl-amine (0.11 ml; 0.63 mmol; 3.00 eq.) in N,N-Dimethyl-formamide (3.00 ml) was stirred at room temperature overnight. The reaction mixture was purified on reverse phase HPLC to obtain 2-[(3R,4S)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile (15.00 mg; 11%). ¹H NMR (400 MHz, Chloroform-d) δ 8.54 (d, J=5.2 Hz, 1H), 8.36 (d, J=2.3 Hz, 1H), 8.29 (dd, J=8.9, 2.3 Hz, 1H), 7.89 (d, J=8.2 Hz, 1H), 7.70 (s, 1H), 7.32-7.19 (m, 2H), 7.12 (d, J=5.2 Hz, 1H), 5.02 (ddd, J=11.5, 5.8, 2.8 Hz, 1H), 4.74 (dd, J=6.6, 2.6 Hz, 4H), 4.54 (q, J=6.6 Hz, 1H), 4.46-4.08 (m, 1H), 3.99 (s, 3H), 3.90-3.45 (m, 4H), 3.18 (s, 4H), 2.64 (d, J=6.9 Hz, 4H), 2.37-2.16 (m, 1H), 1.96 (s, 2H), 1.40 (dd, J=18.5, 6.6 Hz, 3H). MS: m/z=633.3 [M+H]⁺.

### Example 117: 2-[(3S,4R)-3-Fluoro-1-((R)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile: 117

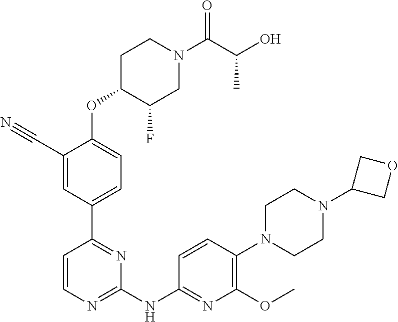 [SMILES: COc1nc(Nc2nccc(-c3ccc(O[C@@H]4CCN(C(=O)[C@@H](C)O)C[C@@H]4F)c(C#N)c3)n2)ccc1N1CCN(C2COC2)CC1]

The title compound was prepared according to the procedures described in example 116 using 4-Chloro-pyrimidin-2-ylamine, (3S,4R)-4-[2-Cyano-4-(4,4,5,5-tetramethyl-[1,3,2]dioxaborolan-2-yl)-phenoxy]-3-fluoro-piperidine-1-carboxylic acid tert-butyl ester, 1-(6-Bromo-2-methoxy-pyridin-3-yl)-4-oxetan-3-yl-piperazine, and (R)-2-Hydroxy-propionic acid. ¹H NMR (400 MHz, Chloroform-d) δ 8.54 (d, J=5.2 Hz, 1H), 8.36 (d, J=2.2 Hz, 1H), 8.28 (dd, J=8.9, 2.2 Hz, 1H), 7.88 (d, J=8.3 Hz, 1H), 7.74 (s, 1H), 7.35-7.16 (m, 2H), 7.11 (d, J=5.2 Hz, 1H), 5.01 (ddt, 7=11.3, 5.5, 2.5 Hz, 1H), 4.87 (s, 1H), 4.80-4.64 (m, 4H), 4.54 (q, J=6.6 Hz, 1H), 4.13 (p, J=6.6, 5.9 Hz, 1H), 3.98 (s, 3H), 3.92-3.50 (m, 3H), 3.13 (s, 4H), 2.58 (t, J=4.8 Hz, 3H), 1.49-1.33 (m, 3H), 1.27 (d, J=10.2 Hz, 4H). MS: m/z=633.3 [M+H]⁺. Example 118: 2-[(3S,4R)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile: 118

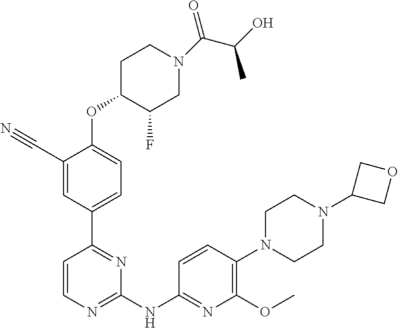 [SMILES: COc1nc(Nc2nccc(-c3ccc(O[C@@H]4CCN(C(=O)[C@H](C)O)C[C@@H]4F)c(C#N)c3)n2)ccc1N1CCN(C2COC2)CC1]

A mixture of 2-((3S,4R)-3-Fluoro-piperidin-4-yloxy)-5-{2-[6-methoxy-5-(4-oxetan-3-yl-piperazin-1-yl)-pyridin-2-ylamino]-pyrimidin-4-yl}-benzonitrile hydrochloride (150.00 mg; 0.18 mmol; 1.00 eq.) (S)-2-Hydroxy-propionic acid (20 mg, 0.22 mmol), [dimethylamino(triazolo[4,5-b]pyridin-3-yloxy)methylene]-dimethyl-ammonium hexafluorophosphate (HATU) (82.53 mg; 0.22 mmol; 1.20 eq.) and Ethyl-diisopropyl-amine (0.09 ml; 0.54 mmol; 3.00 eq.) in N,N-Dimethyl-formamide (3.00 ml) was stirred at room temperature overnight. The crude was purified on reverse phase HPLC to obtain 2-[(3S,4R)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile (30.00 mg; 26%). ¹H NMR (400 MHz, Chloroform-d) δ 8.55 (d, J=5.2 Hz, 1H), 8.37 (d, J=2.2 Hz, 1H), 8.30 (dd, J=8.8, 2.2 Hz, 1H), 7.89 (d, 7=8.2 Hz, 1H), 7.68 (s, 1H), 7.37-7.19 (m, 2H), 7.13 (d, J=5.2 Hz, 1H), 5.08-4.82 (m, 2H), 4.74 (d, J=6.6 Hz, 4H), 4.53 (t, J=13.0 Hz, 2H), 3.99 (s, 3H), 3.87-3.63 (m, 4H), 3.18 (s, 4H), 2.65 (s, 3H), 2.27 (d, J=15.3 Hz, 1H), 2.03-1.86 (m, 1H), 1.56 (s, 3H), 1.41 (d, J=6.6 Hz, 3H). MS: m/z=633.2 [M+H]⁺.

### Example 119: 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile 119

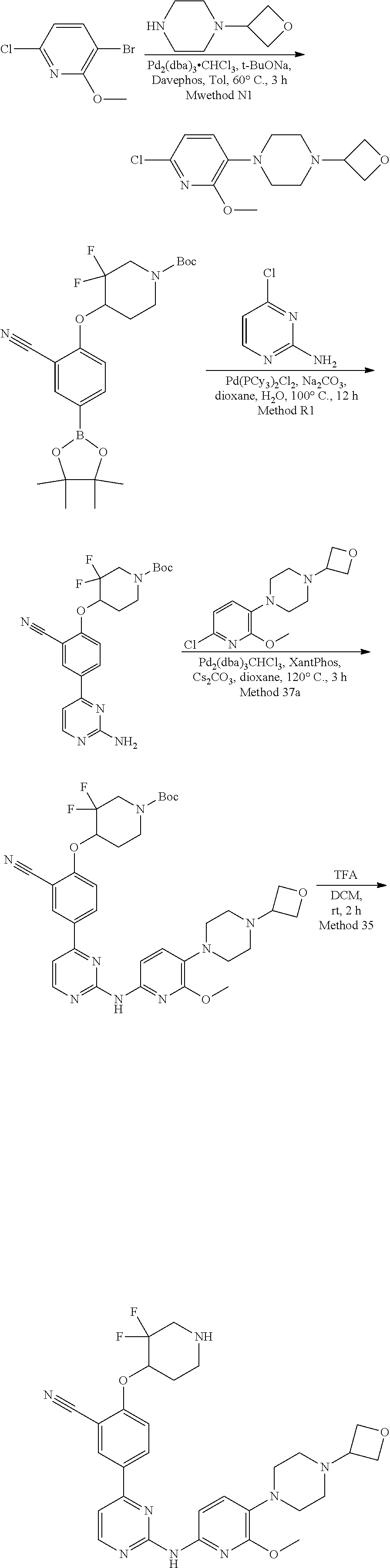

The title compound was prepared from 3-bromo-6-chloro-2-methoxypyridine, 1-(oxetan-3-yl)piperazine, tert-butyl 4-(2-cyano-4-(4,4,5,5-tetramethyl-1,3,2-dioxaborolan-2-yl)phenoxy)-3,3-difluoropiperidine-1-carboxylate, 4-chloropyrimidin-2-amine, 1-(6-chloro-2-methoxypyridin-3-yl)-4-(oxetan-3-yl)piperazine and (S)-2-hydroxypropanoic acid using Method N1, R1, 37a, 35. The final product was purified by prep-HPLC under the following conditions: column, XBridge Prep OBD C18 Column, 150×19 mm, 5 um; mobile phase, acetonitrile in water (with 10 mmol/L NR₄HCO₃ and 0.1% NH₃.H₂O), 30% to 50% gradient in 8 min; detector, UV 254 nm. 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile was obtained as a light yellow solid (308 mg, 12% for 5 steps). HPLC: 98.9% purity, RT=3.47 min. MS: m/z=579.0 [M+H]⁺. ¹H NMR (300 MHz, DMSO-d₆) δ 9.42 (s, 1H), 8.86-8.30 (m, 3H), 7.96-7.41 (m, 3H), 7.33-7.26 (m, 1H), 5.27-5.20 (m, 1H), 4.80-4.34 (m, 4H), 3.95-3.88 (m, 3H), 3.21-2.66 (m, 10H), 2.46-2.20 (m, 3H), 2.18-1.68 (m, 2H).

### Example 120: 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile

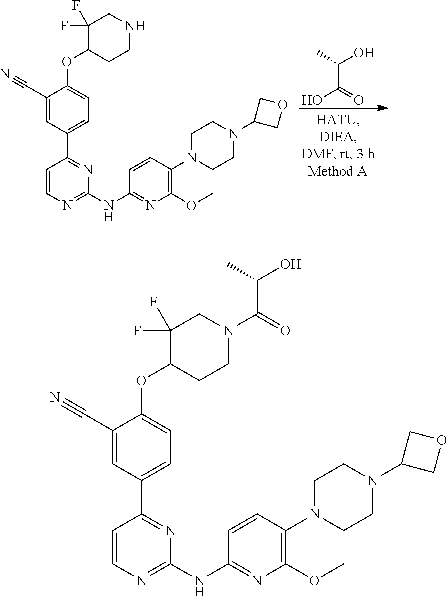

The title compound was prepared from 2-(3,3-Difluoro-piperidin-4-yloxy)-5-{2-[6-methoxy-5-(4-oxetan-3-yl-piperazin-1-yl)-pyridin-2-ylamino]-pyrimidin-4-yl}-benzonitrile and (S)-2-Hydroxy-propionic acid using Method A. The product was purified by prep-HPLC under the following conditions: column, XBridge Prep OBD C18 Column, 150×19 mm, 5 um; mobile phase, acetonitrile in water (with 10 mmol/L NH₄HCO₃ and 0.1% NH₃.H₂O), 30% to 50% gradient in 8 min; detector, UV 254 nm. 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile was obtained as a light yellow solid (308 mg, 12% for 5 steps). HPLC: 98.3% purity, RT=4.24 min. MS: m/z=651.4 [M+H]⁺. ¹H NMR (300 MHz, DMSO-d₆) δ 9.41 (s, 1H), 8.65-8.48 (m, 3H), 7.79-7.63 (m, 2H), 7.55 (d, J=5.3 Hz, 1H), 7.28 (d, J=8.3 Hz, 1H), 5.39 (br s, 1H), 5.29-5.19 (m, 1H), 4.62-4.41 (m, 5H), 4.29-3.54 (m, 7H), 3.53-3.41 (m, 1H), 3.02-2.95 (m, 4H), 2.44-2.38 (m, 4H), 2.25-1.80 (m, 2H), 1.23 (d, J=6.5 Hz, 3H).

### Example 121 & 122: 2-[[(4S)-3,3-difluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile & 2-[[(4R)-3,3-difluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile 121 & 122

The title compounds were obtained by separation of 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile on chiral prep-HPLC under the following conditions: column, CHIRALPAK IC-3, 0.46×10 cm, 3 um; mobile phase, MtBE (with 0.1% DEA) in IPA, 60% isocratic in 30 min; detector, UV 254 nm.

Example 121: (105 mg, 35%, light yellow solid) HPLC: 99.6% purity, RT=4.23 min. MS: m/z=651.1 [M+H]⁺. ¹H NMR (300 MHz, DMSO-d₆) δ 9.42 (s, 1H), 8.69-8.48 (m, 3H), 7.79-7.63 (m, 2H), 7.55 (d, J=5.3 Hz, 1H), 7.28 (d, J=8.3 Hz, 1H), 5.39 (br s, 1H), 5.25 (br s, 1H), 4.62-4.41 (m, 5H), 4.36-3.56 (m, 7H), 3.54-3.41 (m, 1H), 3.02-2.95 (m, 4H), 2.46-2.37 (m, 4H), 2.28-1.77 (m, 2H), 1.23 (d, J=6.4 Hz, 3H).

Example 122: (124 mg, 42%, light yellow solid) HPLC: 99.5% purity, RT=4.22 min. MS: m/z=651.1 [M+H]⁺. ¹H NMR (300 MHz, DMSO-d₆) δ 9.42 (s, 1H), 8.66-8.48 (m, 3H), 7.79-7.63 (m, 2H), 7.55 (d, J=5.3 Hz, 1H), 7.28 (d, J=8.3 Hz, 1H), 5.40 (s, 1H), 5.26 (d, J=6.7 Hz, 1H), 4.62-4.41 (m, 5H), 4.34-3.54 (m, 7H), 3.52-3.41 (m, 1H), 3.01-2.95 (m, 4H), 2.44-2.37 (m, 4H), 2.26-1.76 (m, 2H), 1.22 (d, J=6.2 Hz, 4H).

### Example 123: 2-(3,3-Difluoro-piperidin-4-yloxy)-5-{2-[6-methoxy-5-((S)-3-methyl-4-oxetan-3-yl-piperazin-1-yl)-pyridin-2-ylamino]-pyrimidin-4-yl}-benzonitrile 123

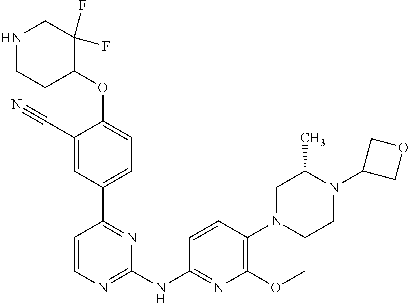 [SMILES: COc1nc(Nc2nccc(-c3ccc(OC4CCNCC4(F)F)c(C#N)c3)n2)ccc1N1CCN(C2COC2)[C@@H](C)C1]

### (S)-4-(6-Bromo-2-methoxy-pyridin-3-yl)-2-methyl-1-oxetan-3-yl-piperazine

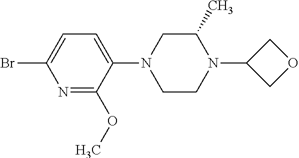 [SMILES: COc1nc(Br)ccc1N1CCN(C2COC2)[C@@H](C)C1]

To a solution of (S)-1-(6-Bromo-2-methoxy-pyridin-3-yl)-3-methyl-piperazine (4000.00 mg; 13.98 mmol; 1.00 eq.), Oxetan-3-one (2014.56 mg; 27.96 mmol; 2.00 eq.) and Acetic acid (167.88 mg; 2.80 mmol; 0.20 eq.) in THF (30 mL) was stirred at room temperature for overnight. Sodium triacetoxyborohydride (8887.40 mg; 41.93 mmol; 3.00 eq.) was added and the mixture was stirred for another 3 hours. The solvent was removed and. the crude product was purified through flash chromatography on silica gel (EtOAc in Hexanes from 0% to 50% containing 0.1% triethylamine) to provide (S)-4-(6-Bromo-2-methoxy-pyridin-3-yl)-2-methyl-1-oxetan-3-yl-piperazine (3800.00 mg; 11.10 mmol) in 79% yield, m/z: 343 (M+H)⁺.

### 4-(2-Cyano-4-{2-[6-methoxy-5-((S)-3-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester

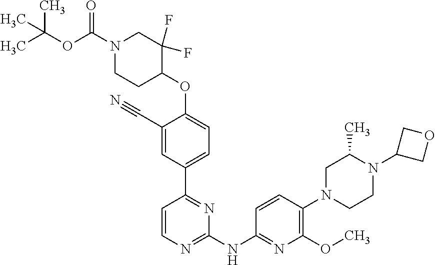 [SMILES: COc1nc(Nc2nccc(-c3ccc(OC4CCN(C(=O)OC(C)(C)C)CC4(F)F)c(C#N)c3)n2)ccc1N1CCN(C2COC2)[C@@H](C)C1]

A mixture of 4-[4-(2-Amino-pyrimidin-4-yl)-2-cyano-phenoxy]-3,3-difluoro-piperidine-1-carboxylic acid tert-butyl ester (200.00 mg; 0.46 mmol; 1.00 eq.), (S)-4-(6-Bromo-2-methoxy-pyridin-3-yl)-2-methyl-1-oxetan-3-yl-piperazine (158.65 mg; 0.46 mmol; 1.00 eq.), 4,5-Bis-diphenylphosphanyl-9,9-dimethyl-9H-xanthene (0.09 ml; 0.14 mmol; 0.30 eq.), and Cs2CO3 (317.98 mg; 0.93 mmol; 2.00 eq.) in Dioxane in a microwave vial was purged with argon for 3 min. Then Pd2(dba)₃CHCl3 (101.02 mg; 0.09 mmol; 0.20 eq.) was added. The reaction mixture was heated at 100° C. for overnight, filtered, and the solvent was removed and the residue was purified by flash chromatography on silica gel (Hex: EtOAc from 50:50 to 0:100, then, MeOH in EtOAc from 0% to 15%) to provide 4-(2-Cyano-4-{2-[6-methoxy-5-((S)-3-methy 1-4-oxetan-3-yl-piperazin-1-yl)-pyridin-2-ylamino]-pyrimidin-4-yl}-phenoxy)-3,3-difluoro-piperidine-1-carboxylic acid tert-butyl ester (260.00 mg; 0.38 mmol) in 81% yield, m/z: 693 (M+H)⁺.

### 2-(3,3-Difluoro-piperidin-4-yloxy)-5-{2-[6-methoxy-5-((S)-3-methyl-4-oxetan-3-yl-piperazin-1-yl)-pyridin-2-ylamino]-pyrimidin-4-yl}-benzonitrile

The title compound (340 mg) was synthesized using 4-(2-Cyano-4-{2-[6-methoxy-5-((S)-3-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester (1300.00 mg; 1.88 mmol; 1.00 eq.) and HCl in Dioxane (4M, 10 mL) in 30% yield, m/z: 593 (M+H). ¹H NMR (DMSO-d6): 9.41 (1H), 8.62 (2H), 8.51 (1H), 7.76 (1H), 7.67 (1H), 7.55 (1H), 7.34 (1H), 5.22 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 0.82 (3H).

### Example 124: 2-(3,3-Difluoro-piperidin-4-yloxy)-5-{2-[6-methoxy-5-((R)-3-methyl-4-oxetan-3-yl-piperazin-1-yl)-pyridin-2-ylamino]-pyrimidin-4-yl}-benzonitrile 124

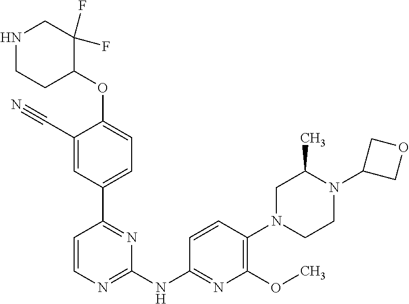 [SMILES: COc1nc(Nc2nccc(-c3ccc(OC4CCNCC4(F)F)c(C#N)c3)n2)ccc1N1CCN(C2COC2)[C@H](C)C1]

### (R)-4-(6-Bromo-2-methoxy-pyridin-3-yl)-2-methyl-1-oxetan-3-yl-piperazine

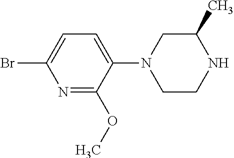 [SMILES: COc1nc(Br)ccc1N1CCN[C@H](C)C1]

To a solution of (S)-1-(6-Bromo-2-methoxy-pyridin-3-yl)-3-methyl-piperazine (4000.00 mg; 13.98 mmol; 1.00 eq.), Oxetan-3-one (2014.56 mg; 27.96 mmol; 2.00 eq.) and Acetic acid (167.88 mg; 2.80 mmol; 0.20 eq.) in THF (30 mL) was stirred at room temperature for overnight. Sodium triacetoxyborohydride (8887.40 mg; 41.93 mmol; 3.00 eq.) was added and the mixture was stirred for another 3 hours. The crude product was purified through flash chromatography on silica gel (EtOAc in Hexanes from 0% to 50% containing 0.1% triethylamine) to provide (S)-4-(6-Bromo-2-methoxy-pyridin-3-yl)-2-methyl-1-oxetan-3-yl-piperazine (4100.00 mg; 11.10 mmol) in 81% yield, m/z: 343 (M+H)⁺.

### 4-(2-Cyano-4-{2-[6-methoxy-5-((R)-3-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester

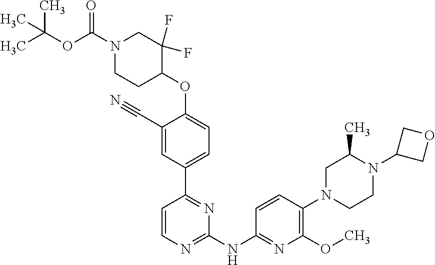 [SMILES: COc1nc(Nc2nccc(-c3ccc(OC4CCN(C(=O)OC(C)(C)C)CC4(F)F)c(C#N)c3)n2)ccc1N1CCN(C2COC2)[C@H](C)C1]

A mixture of 4-[4-(2-Amino-pyrimidin-4-yl)-2-cyano-phenoxy]-3,3-difluoro-piperidine-1-carboxylic acid tert-butyl ester (200.00 mg; 0.46 mmol; 1.00 eq.), (R)-4-(6-Bromo-2-methoxy-pyridin-3-yl)-2-methyl-1-oxetan-3-yl-piperazine (158.65 mg; 0.46 mmol; 1.00 eq.), 4,5-Bis-diphenylphosphanyl-9,9-dimethyl-9H-xanthene (0.09 ml; 0.14 mmol; 0.30 eq.), and Cs2CO3 (317.98 mg; 0.93 mmol; 2.00 eq.) in Dioxane in a microwave vial was purged with argon for 3 min. Then Pd2(dba)₃CHCl3 (101.02 mg; 0.09 mmol; 0.20 eq.) was added. The reaction mixture was heated at 100° C. for overnight. Filtered and the solvent was removed and the residue was dissolved in EtOAc and loaded at flash chromatography on silica gel (Hex: EtOAc from 50:50 to 0:100, then, MeOH in EtOAc from 0% to 15%) to provide the product. 4-(2-Cyano-4-{2-[6-methoxy-5-((S)-3-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester (240.00 mg; 0.38 mmol) in 75% yield, m/z: 693 (M+H)⁺.

### 2-(3,3-Difluoro-piperidin-4-yloxy)-5-{2-[6-methoxy-5-((R)-3-methyl-4-oxetan-3-yl-piperazin-1-yl)-pyridin-2-ylamino]-pyrimidin-4-yl}-benzonitrile

The title compound (220 mg) was synthesized using 4-(2-Cyano-4-{2-[6-methoxy-5-((R)-3-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester (1150.00 mg; 1.88 mmol; 1.00 eq.) and HCl in Dioxane (4M, 10 mL) in 21% yield, m/z: 593 (M+H). ¹H NMR (DMSO-d6): 9.41 (1H), 8.62 (2H), 8.51 (1H), 7.76 (1H), 7.67 (1H), 7.55 (1H), 7.34 (1H), 5.22 (1H), 3.89 (3H), 3.13 (1H), 3.04 (1H), 2.89 (2H), 2.67 (2H), 2.58 (1H), 2.38 (1H), 2.20 (3H), 2.05 (2H), 1.90 (2H), 0.82 (3H).

### Example 125: 2-[3,3-Difluoro-1-((R)-2-hydroxy-propionyl)-piperidin-4-yloxy]-5-{2-[6-methoxy-5-((S)-3-methyl-4-oxetan-3-yl-piperazin-1-yl)-pyridin-2-ylamino]-pyrimidin-4-yl}-benzonitrile 125

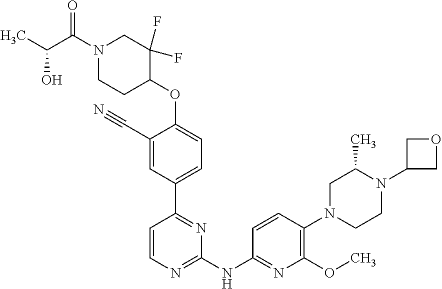 [SMILES: COc1nc(Nc2nccc(-c3ccc(OC4CCN(C(=O)[C@@H](C)O)CC4(F)F)c(C#N)c3)n2)ccc1N1CCN(C2COC2)[C@@H](C)C1]

The title compound (41.7 mg) was synthesized using 2-(3,3-Difluoro-piperidin-4-yloxy)-5-{2-[6-methoxy-5-((S)-3-methyl-4-oxetan-3-yl-piperazin-1-yl)-pyridin-2-ylamino]-pyrimidin-4-yl}-benzonitrile (70.00 mg; 0.12 mmol; 1.00 eq.), (R)-2-Hydroxy-propionic acid (21.28 mg; 0.24 mmol; 2.00 eq.), O-(7-Azabenzotriazol-1-yl)-N,N,N′,N′-tetramethyluronium hexaflurophosphate (HATU) (78.80 mg; 0.21 mmol; 1.75 eq.) and Ethyl-diisopropyl-amine (76.33 mg; 0.59 mmol; 5.00 eq.) in 50% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 126: 2-[3,3-Difluoro-1-((R)-2-hydroxy-propionyl)-piperidin-4-yloxy]-5-{2-[6-methoxy-5-((R)-3-methyl-4-oxetan-3-yl-piperazin-1-yl)-pyridin-2-ylamino]-pyrimidin-4-yl}-benzonitrile 126

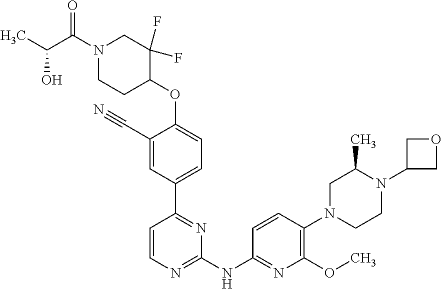 [SMILES: COc1nc(Nc2nccc(-c3ccc(OC4CCN(C(=O)[C@@H](C)O)CC4(F)F)c(C#N)c3)n2)ccc1N1CCN(C2COC2)[C@H](C)C1]

The title compound (31 mg) was synthesized using 2-(3,3-Difluoro-piperidin-4-yloxy)-5-{2-[6-methoxy-5-((R)-3-methyl-4-oxetan-3-yl-piperazin-1-yl)-pyridin-2-ylamino]-pyrimidin-4-yl}-benzonitrile (70.00 mg; 0.12 mmol; 1.00 eq.), (R)-2-Hydroxy-propionic acid (21.28 mg; 0.24 mmol; 2.00 eq.), O-(7-Azabenzotriazol-1-yl)-N,N,N′,N′-tetramethyluronium hexaflurophosphate (HATU) (78.80 mg; 0.21 mmol; 1.73 eq.) and Ethyl-diisopropyl-amine (76.33 mg; 0.59 mmol; 5.00 eq.) in 38% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 127: 2-[3,3-Difluoro-1-((S)-2-hydroxy-propionyl)-piperidin-4-yloxy]-5-{2-[6-methoxy-5-((S)-3-methyl-4-oxetan-3-yl-piperazin-1-yl)-pyridin-2-ylamino]-pyrimidin-4-yl}-benzonitrile 127

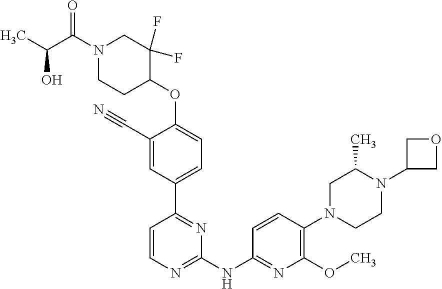 [SMILES: COc1nc(Nc2nccc(-c3ccc(OC4CCN(C(=O)[C@H](C)O)CC4(F)F)c(C#N)c3)n2)ccc1N1CCN(C2COC2)[C@@H](C)C1]

The title compound (36.5 mg) was synthesized using 2-(3,3-Difluoro-piperidin-4-yloxy)-5-{2-[6-methoxy-5-((S)-3-methyl-4-oxetan-3-yl-piperazin-1-yl)-pyridin-2-ylamino]-pyrimidin-4-yl}-benzonitrile (70.00 mg; 0.12 mmol; 1.00 eq.), (S)-2-Hydroxy-propionic acid (21.28 mg; 0.24 mmol; 2.00 eq.), O-(7-Azabenzotriazol-1-yl)-N,N,N′,N′-tetramethyluronium hexaflurophosphate (HATU) (78.80 mg; 0.21 mmol; 1.75 eq.) and Ethyl-diisopropyl-amine (76.33 mg; 0.59 mmol; 5.00 eq.). in 44% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 128: 2-[3,3-Difluoro-1-((S)-2-hydroxy-propionyl)-piperidin-4-yloxy]-5-{2-[6-methoxy-5-((R)-3-methyl-4-oxetan-3-yl-piperazin-1-yl)-pyridin-2-ylamino]-pyrimidin-4-yl}-benzonitrile 128

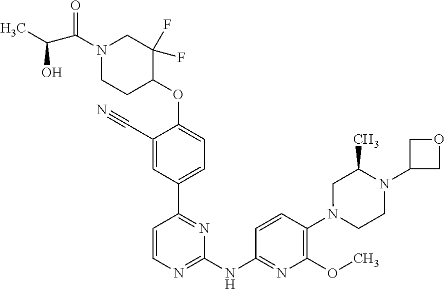 [SMILES: COc1nc(Nc2nccc(-c3ccc(OC4CCN(C(=O)[C@H](C)O)CC4(F)F)c(C#N)c3)n2)ccc1N1CCN(C2COC2)[C@H](C)C1]

The title compound (42 mg) was synthesized using 2-(3,3-Difluoro-piperidin-4-yloxy)-5-{2-[6-methoxy-5-((R)-3-methyl-4-oxetan-3-yl-piperazin-1-yl)-pyridin-2-ylamino]-pyrimidin-4-yl}-benzonitrile (70.00 mg; 0.12 mmol; 1.00 eq.), (S)-2-Hydroxy-propionic acid (21.28 mg; 0.24 mmol; 2.00 eq.), O-(7-Azabenzotriazol-1-yl)-N,N,N′,N′-tetramethyluronium hexaflurophosphate (HATU) (78.80 mg; 0.21 mmol; 1.75 eq.) and Ethyl-diisopropyl-amine (76.33 mg; 0.59 mmol; 5.00 eq.). in 51% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 129: 2-(3,3-Difluoro-piperidin-4-yloxy)-5-{2-[6-methoxy-5-((R)-2-methyl-4-oxetan-3-yl-piperazin-1-yl)-pyridin-2-ylamino]-pyrimidin-4-yl}-benzonitrile 129

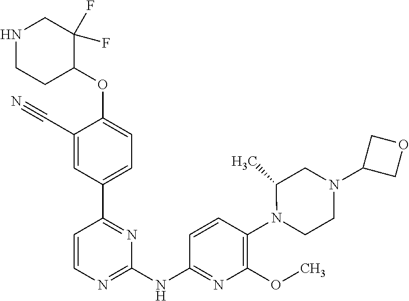 [SMILES: COc1nc(Nc2nccc(-c3ccc(OC4CCNCC4(F)F)c(C#N)c3)n2)ccc1N1CCN(C2COC2)C[C@H]1C]

### (R)-4-(6-Bromo-2-methoxy-pyridin-3-yl)-2-methyl-4-oxetan-3-yl-piperazine

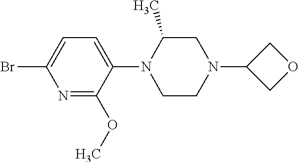 [SMILES: COc1nc(Br)ccc1N1CCN(C2COC2)C[C@H]1C]

To a solution of (R)-1-(6-Bromo-2-methoxy-pyridin-3-yl)-3-methyl-piperazine (2250.00 mg; 13.98 mmol; 1.00 eq.), Oxetan-3-one (1133.56 mg; 27.96 mmol; 2.00 eq.) and Acetic acid (94.88 mg; 2.80 mmol; 0.20 eq.) in THF (30 mL) was stirred at room temperature for overnight. Sodium triacetoxyborohydride (4999.40 mg; 41.93 mmol; 3.00 eq.) was added and the mixture was stirred for another 3 hours. The solvent was removed and the crude product was purified through flash chromatography on silica gel (EtOAc in Hexanes from 0% to 50% containing 0.1% triethylamine) to provide (R)-4-(6-Bromo-2-methoxy-pyridin-3-yl)-2-methyl-4-oxetan-3-yl-piperazine (4100.00 mg; 11.10 mmol) in 81% yield, m/z: 343 (M+H)⁺.

### 4-(2-Cyano-4-{2-[6-methoxy-5-((R)-2-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester

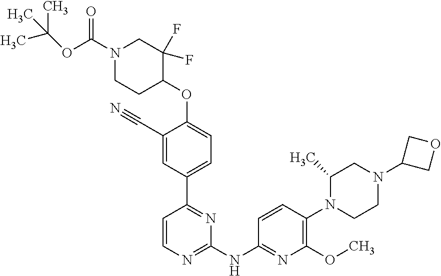 [SMILES: COc1nc(Nc2nccc(-c3ccc(OC4CCN(C(=O)OC(C)(C)C)CC4(F)F)c(C#N)c3)n2)ccc1N1CCN(C2COC2)C[C@H]1C]

A mixture of 4-[4-(2-Amino-pyrimidin-4-yl)-2-cyano-phenoxy]-3,3-difluoro-piperidine-1-carboxylic acid tert-butyl ester (200.00 mg; 0.46 mmol; 1.00 eq.), (R)-4-(6-Bromo-2-methoxy-pyridin-3-yl)-2-methyl-4-oxetan-3-yl-piperazine (134.65 mg; 0.46 mmol; 1.00 eq.), 4,5-Bis-diphenylphosphanyl-9,9-dimethyl-9H-xanthene (101 mg; 0.14 mmol; 0.30 eq.), and Cs2CO3 (317.98 mg; 0.93 mmol; 2.00 eq.) in Dioxane in a microwave vial was purged with argon for 3 min. Then Pd2(dba)₃CHCl3 (101.02 mg; 0.09 mmol; 0.20 eq.) was added. The reaction mixture was heated at 100° C. for overnight. Filtered and the solvent was removed. The residue was purified by chromatography on silica gel (Hex: EtOAc from 50:50 to 0:100, then, MeOH in EtOAc from 0% to 15%) to provide the product. 4-(2-Cyano-4-{2-[6-methoxy-5-((R)-2-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester (270.00 mg; 0.38 mmol) in 84% yield, m/z: 693 (M+H)⁺.

### 2-(3,3-Difluoro-piperidin-4-yloxy)-5-{2-[6-methoxy-5-((R)-2-methyl-4-oxetan-3-yl-piperazin-1-yl)-pyridin-2-ylamino]-pyrimidin-4-yl}-benzonitrile

The title compound (300 mg) was synthesized using 4-(2-Cyano-4-{2-[6-methoxy-5-((R)-2-methyl-4-oxetan-3-yl-piperazin-1-yl)-pyridin-2-ylamino]-pyrimidin-4-yl}-phenoxy)-3,3-difluoro-piperidine-1-carboxylic acid tert-butyl ester (1350.00 mg; 1.95 mmol; 1.00 eq.) and HCl in Dioxane (4M, 20 mL) in 23% yield, m/z: 593 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (1H), 4.50 (2H), 3.92 (3H), 3.43 (2H), 3.17 (1H), 3.04 (1H), 2.89 (2H), 2.74 (2H), 2.58 (1H), 2.38 (1H), 2.30 (2H), 1.90 (1H), 1.86 (1H), 0.82 (3H).

### Example 130: 2-[3,3-Difluoro-1-((R)-2-hydroxy-propionyl)-piperidin-4-yloxy]-5-{2-[6-methoxy-5-((R)-2-methyl-4-oxetan-3-yl-piperazin-1-yl)-pyridin-2-ylamino]-pyrimidin-4-yl}-benzonitrile 130

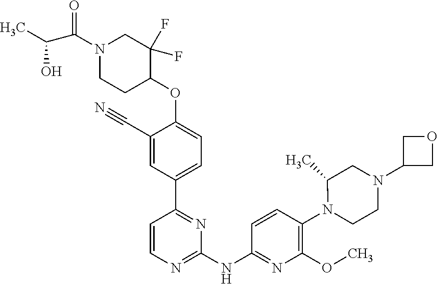 [SMILES: COc1nc(Nc2nccc(-c3ccc(OC4CCN(C(=O)[C@@H](C)O)CC4(F)F)c(C#N)c3)n2)ccc1N1CCN(C2COC2)C[C@H]1C]

The title compound (22.5 mg) was prepared using 2-(3,3-Difluoro-piperidin-4-yloxy)-5-{2-[6-methoxy-5-((R)-3-methyl-4-oxetan-3-yl-piperazin-1-yl)-pyridin-2-ylamino]-pyrimidin-4-yl}-benzonitrile (80.00 mg; 0.12 mmol; 1.00 eq.), (R)-2-Hydroxy-propionic acid (21.28 mg; 0.24 mmol; 2.00 eq.), O-(7-Azabenzotriazol-1-yl)-N,N,N′,N′-tetramethyluronium hexaflurophosphate (HATU) (78.80 mg; 0.21 mmol; 1.75 eq.) and Ethyl-diisopropyl-amine (76.33 mg; 0.59 mmol; 5.00 eq.) using Method A in 21% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 131: 2-[3,3-Difluoro-1-((S)-2-hydroxy-propionyl)-piperidin-4-yloxy]-5-{2-[6-methoxy-5-((S)-2-methyl-4-oxetan-3-yl-piperazin-1-yl)-pyridin-2-ylamino]-pyrimidin-4-yl}-benzonitrile 131

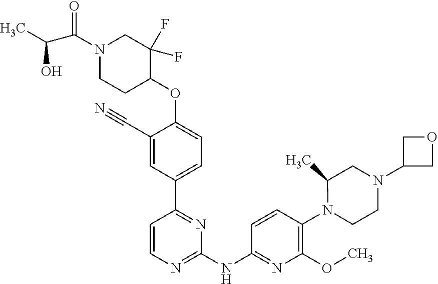 [SMILES: COc1nc(Nc2nccc(-c3ccc(OC4CCN(C(=O)[C@H](C)O)CC4(F)F)c(C#N)c3)n2)ccc1N1CCN(C2COC2)C[C@@H]1C]

The title compound (4.1 mg) was synthesized using 2-(3,3-Difluoro-piperidin-4-yloxy)-5-{2-[6-methoxy-5-((S)-2-methyl-4-oxetan-3-yl-piperazin-1-yl)-pyridin-2-ylamino]-pyrimidin-4-yl}-benzonitrile (50.00 mg; 0.12 mmol; 1.00 eq.), (S)-2-Hydroxy-propionic acid (15.28 mg; 0.24 mmol; 2.00 eq.), O-(7-Azabenzotriazol-1-yl)-N,N,N′,N′-tetramethyluronium hexaflurophosphate (HATU) (56.80 mg; 0.21 mmol; 1.75 eq.) and Ethyl-diisopropyl-amine (54.33 mg; 0.59 mmol; 5.00 eq.) using Method A in 6.7% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 132: 2-[3,3-Difluoro-1-((S)-2-hydroxy-propionyl)-piperidin-4-yloxy]-5-{2-[6-methoxy-5-((R)-2-methyl-4-oxetan-3-yl-piperazin-1-yl)-pyridin-2-ylamino]-pyrimidin-4-yl}-benzonitrile 132

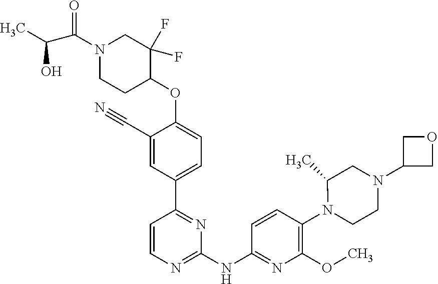 [SMILES: COc1nc(Nc2nccc(-c3ccc(OC4CCN(C(=O)[C@H](C)O)CC4(F)F)c(C#N)c3)n2)ccc1N1CCN(C2COC2)C[C@H]1C]

The title compound (24.9 mg) was synthesized using 2-(3,3-Difluoro-piperidin-4-yloxy)-5-{2-[6-methoxy-5-((R)-2-methyl-4-oxetan-3-yl-piperazin-1-yl)-pyridin-2-ylamino]-pyrimidin-4-yl}-benzonitrile (80.00 mg; 0.12 mmol; 1.00 eq.), (S)-2-Hydroxy-propionic acid (21.28 mg; 0.24 mmol; 2.00 eq.), O-(7-Azabenzotriazol-1-yl)-N,N,N′,N′-tetramethyluronium hexaflurophosphate (HATU) (78.80 mg; 0.21 mmol; 1.73 eq.) and Ethyl-diisopropyl-amine (76.33 mg; 0.59 mmol; 5.00 eq.) using Method A in 23% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 133: 2-[1-((S)-2,3-Dihydroxy-propionyl)-3,3-difluoro-piperidin-4-yloxy]-5-{2-[6-methoxy-5-((R)-2-methyl-4-oxetan-3-yl-piperazin-1-yl)-pyridin-2-ylamino]-pyrimidin-4-yl}-benzonitrile 133

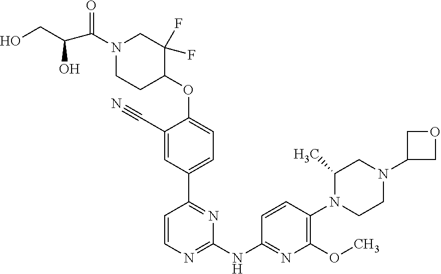 [SMILES: COc1nc(Nc2nccc(-c3ccc(OC4CCN(C(=O)[C@@H](O)CO)CC4(F)F)c(C#N)c3)n2)ccc1N1CCN(C2COC2)C[C@H]1C]

The title compound (13.1 mg) was synthesized using 2-(3,3-Difluoro-piperidin-4-yloxy)-5-{2-[6-methoxy-5-((R)-2-methyl-4-oxetan-3-yl-piperazin-1-yl)-pyridin-2-ylamino]-pyrimidin-4-yl}-benzonitrile (80.00 mg; 0.12 mmol; 1.00 eq.), (S)-2,3-Dihydroxy-propionic acid (25.06 mg; 0.24 mmol; 2.00 eq.), 0-(7-Azabenzotriazol-1-yl)-N,N,N′,N′-tetramethyluronium hexaflurophosphate (HATU) (78.80 mg; 0.21 mmol; 1.75 eq.) and Ethyl-diisopropyl-amine (76.33 mg; 0.59 mmol; 5.00 eq.) in 15% yield, m/z: 681 (M+H). ¹H NMR (DMSO-d6): H NMR (DMSO-d6): 9.49 (1H), 8.61 (2H), 8.51 (1H), 7.78 (1H), 7.65 (1H), 7.56 (1H), 7.39 (1H), 5.41 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 2.02 (2H), 0.82 (3H).

### Example 134: 5-{2-[4-(4-Cyclopropyl-piperazin-1-yl)-phenylamino]-pyrimidin-4-yl}-2-(tetrahydro-pyran-4-yloxy)-benzonitrile 134

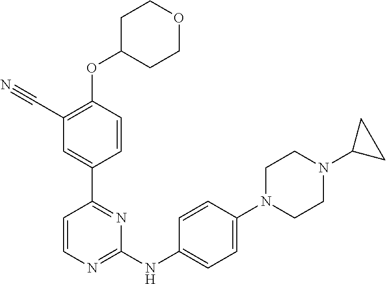 [SMILES: N#Cc1cc(-c2ccnc(Nc3ccc(N4CCN(C5CC5)CC4)cc3)n2)ccc1OC1CCOCC1]

The title compound was prepared from 5-(2-Chloro-pyrimidin-4-yl)-2-(tetrahydro-pyran-4-yloxy)-benzonitrile (150.00 mg; 0.48 mmol; 1.00 eq.), 4-(4-Cyclopropyl-piperazin-1-yl)-phenylamine (103.23 mg; 0.48 mmol; 1.00 eq.), using Method 28 as a white solid (40 mg, 17%). m/z: 497.8 (M+H)⁺.

### Example 135: 2-(oxan-4-yloxy)-5-[2-([4-[4-(oxetan-3-yl)piperazin-1-yl]phenyl]amino)pyrimidin-4-yl]benzonitrile 135

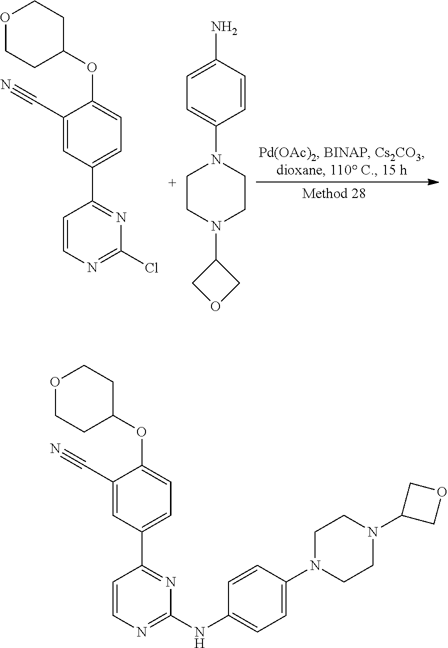

The title compound was prepared from 5-(2-chloropyrimidin-4-yl)-2-(oxan-4-yloxy)benzonitrile and 4-[4-(oxetan-3-yl)piperazin-1-yl]aniline using Method 28. The final product was purified by prep-HPLC under the following conditions: column, XBridge Prep C18 OBD Column, 150×19 mm, 5 um; mobile phase, acetonitrile in water (with 10 mmol/L NR₄HCO₃ and 0.1% NH₃.H₂O), 30% to 47% gradient in 8 min; detector, UV 254 nm. 2-(oxan-4-yloxy)-5-[2-([4-[4-(oxetan-3-yl)piperazin-1-yl]phenyl]amino)pyrimidin-4-yl]benzonitrile was obtained as an yellow solid (37 mg, 17%). HPLC: 98.1% purity, RT=3.03 min. MS: m/z=513.2 [M+H]⁺. ¹H NMR (300 MHz, DMSO-d₆) δ 9.42 (s, 1H), 8.54-8.37 (m, 3H), 7.69-7.48 (m, 3H), 7.42-7.34 (m, 1H), 6.98-6.86 (m, 2H), 5.01-4.87 (m, 1H), 4.61-4.42 (m, 4H), 3.94-3.81 (m, 2H), 3.62-3.39 (m, 3H), 3.15-3.05 (m, 4H), 2.46-2.36 (m, 4H), 2.11-1.98 (m, 2H), 1.78-1.60 (m, 2H).

### Example 136: 2-[(3,3-difluoropiperidin-4-yl)oxy]-5-[2-([4-[4-(oxetan-3-yl)piperazin-1-yl]phenyl]amino)pyrimidin-4-yl]benzonitrile 136

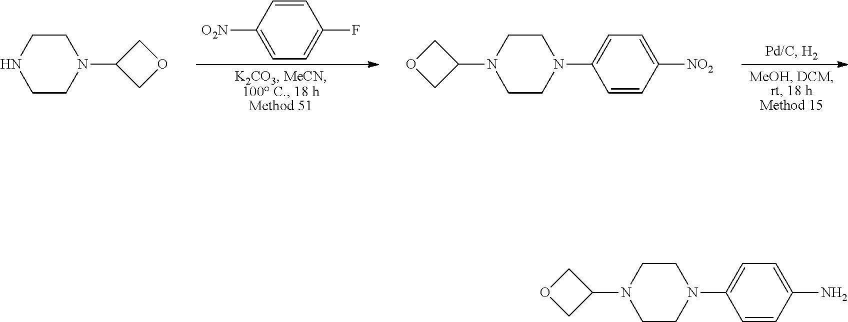

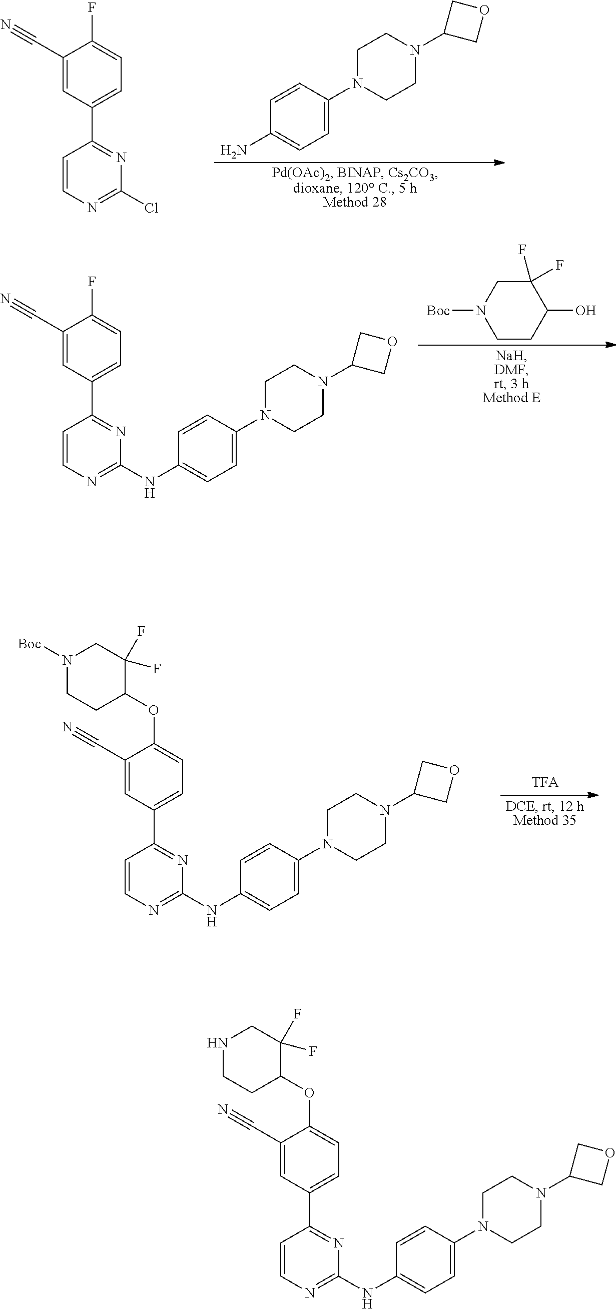

The title compound was prepared from 1-(oxetan-3-yl)piperazine, 1-fluoro-4-nitrobenzene, 5-(2-chloropyrimidin-4-yl)-2-fluorobenzonitrile and tert-butyl 3,3-difluoro-4-hydroxypiperidine-1-carboxylate using Method 51, 15, 28, E and 35. The final product was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 30% to 42% gradient in 8 min; detector, UV 254 nm. 2-[(3,3-difluoropiperidin-4-yl)oxy]-5-[2-([4-[4-(oxetan-3-yl)piperazin-1-yl]phenyl]amino)pyrimidin-4-yl]benzonitrile was obtained as an yellow solid (20 mg, 8.8% for 5 steps). HPLC: 99.3% purity, RT=3.15 min. MS: m/z=548.1 [M+H]⁺. ¹H NMR (300 MHz, DMSO-d₆) δ 9.39 (s, 1H), 8.56-8.36 (m, 3H), 7.63-7.52 (m, 3H), 7.40-7.31 (m, 1H), 6.93-6.82 (m, 2H), 5.23-5.09 (m, 1H), 4.62-4.37 (m, 4H), 3.49-3.34 (m, 1H), 3.22-2.49 (m, 9H), 2.42-2.32 (m, 4H), 2.08-1.71 (m, 2H).

### Example 137: 2-[[3,3-difluoro-1-(2-hydroxyacetyl)piperidin-4-yl]oxy]-5-[2-([4-[4-(oxetan-3-yl)piperazin-1-yl]phenyl]amino)pyrimidin-4-yl]benzonitrile 137

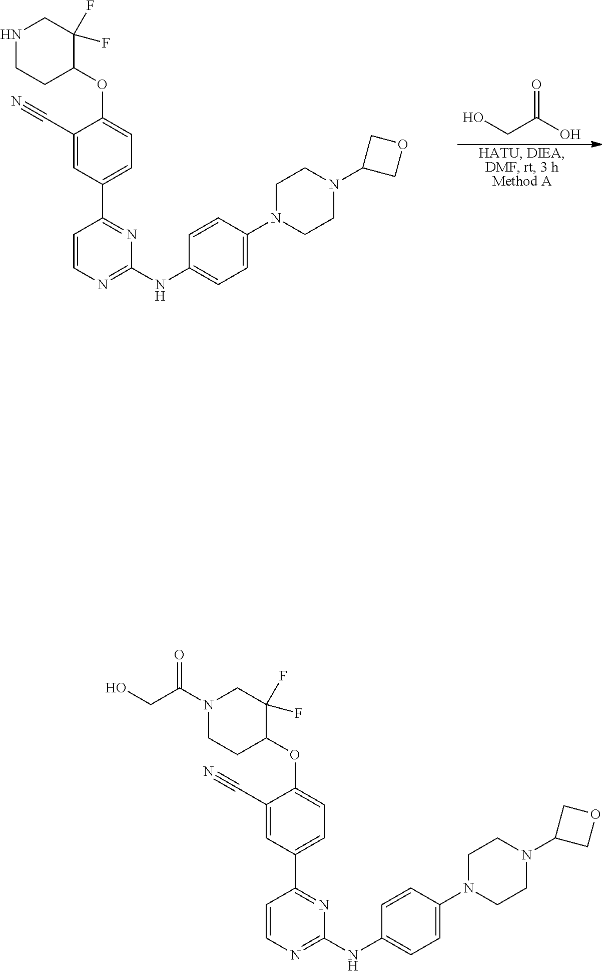

The title compound was prepared from 2-[(3,3-difluoropiperidin-4-yl)oxy]-5-[2-([4-[4-(oxetan-3-yl)piperazin-1-yl]phenyl]amino)pyrimidin-4-yl]benzonitrile and 2-hydroxyacetic acid using Method A. The final product was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 32% to 39% gradient in 8 min; detector, UV 254 nm. 2-[[3,3-difluoro-1-(2-hydroxyacetyl)piperidin-4-yl]oxy]-5-[2-([4-[4-(oxetan-3-yl)piperazin-1-yl]phenyl]amino)pyrimidin-4-yl]benzonitrile was obtained as an yellow solid (25 mg, 24%). HPLC: 99.3% purity, RT=1.11 min. MS: m/z=606.2 [M+H]⁺. ¹H NMR (300 MHz, DMSO-d₆) δ 9.45 (s, 1H), 8.63-8.42 (m, 3H), 7.73-7.54 (m, 3H), 7.48-7.36 (m, 1H), 7.01-6.85 (m, 2H), 5.45-5.29 (m, 1H), 4.98-4.81 (m, 1H), 4.65-4.42 (m, 4H), 4.27-3.39 (m, 7H), 3.20-3.03 (m, 4H), 2.45-2.35 (m, 4H), 2.25-1.80 (m, 2H).

### Example 138: 2-([3,3-difluoro-1-[(2S)-2-hydroxypropanoyl]piperidin-4-yl]oxy)-5-[2-([4-[4-(oxetan-3-yl)piperazin-1-yl]phenyl]amino)pyrimidin-4-yl]benzonitrile 138

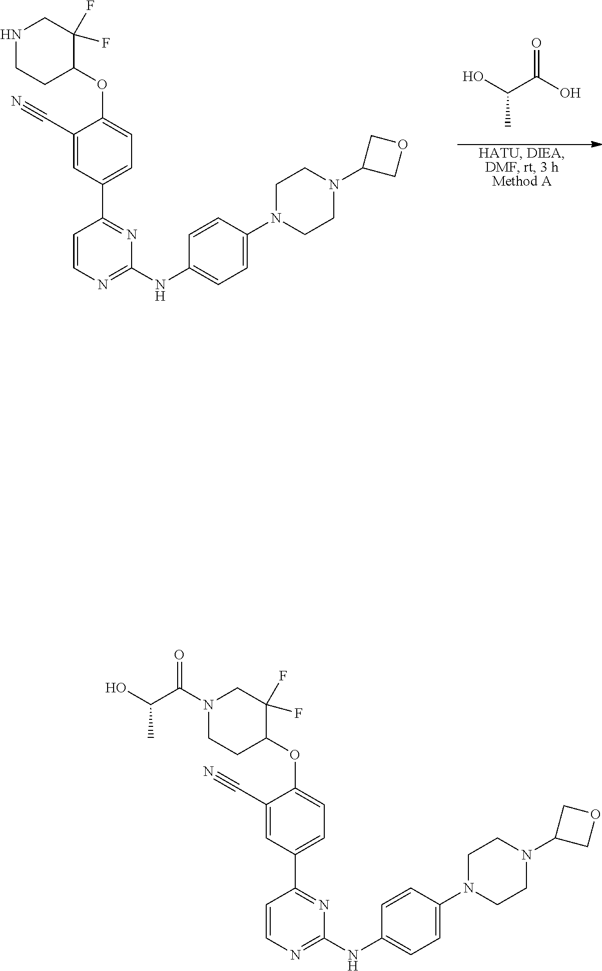

The title compound was prepared from 2-[(3,3-difluoropiperidin-4-yl)oxy]-5-[2-([4-[4-(oxetan-3-yl)piperazin-1-yl]phenyl]amino)pyrimidin-4-yl]benzonitrile and (2S)-2-hydroxypropanoic acid using Method A. The final product was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 31% to 35% gradient in 8 min; detector, UV 254 nm. 2-([3,3-difluoro-1-[(2S)-2-hydroxypropanoyl]piperidin-4-yl]oxy)-5-[2-([4-[4-(oxetan-3-yl)piperazin-1-yl]phenyl]amino)pyrimidin-4-yl]benzonitrile was obtained as a light yellow solid (33 mg, 31%). HPLC: 97.9% purity, RT=3.93 min. MS: m/z=620.2 [M+H]⁺. ¹H NMR (300 MHz, DMSO-d₆) δ 9.43 (s, 1H), 8.58-8.42 (m, 3H), 7.71-7.57 (m, 3H), 7.44-7.36 (m, 1H), 6.97-6.87 (m, 2H), 5.50-5.13 (m, 2H), 4.63-4.42 (m, 5H), 4.34-3.37 (m, 5H), 3.15-3.06 (m, 4H), 2.46-2.37 (m, 4H), 2.25-1.75 (m, 2H), 1.23 (d, J=6.5 Hz, 3H).

### Example 139 and Example 140: 2-[[(4S)-3,3-difluoro-1-[(2S)-2-hydroxypropanoyl]piperidin-4-yl]oxy]-5-[2-([4-[4-(oxetan-3-yl)piperazin-1-yl]phenyl]amino)pyrimidin-4-yl]benzonitrile & 2-[[(4R)-3,3-difluoro-1-[(2S)-2-hydroxypropanoyl]piperidin-4-yl]oxy]-5-[2-([4-[4-(oxetan-3-yl)piperazin-1-yl]phenyl]amino)pyrimidin-4-yl]benzonitrile 139 & 140

The two diastereomers were obtained by separation of 2-([3,3-difluoro-1-[(2S)-2-hydroxypropanoyl]piperidin-4-yl]oxy)-5-[2-([4-[4-(oxetan-3-yl)piperazin-1-yl]phenyl]amino)pyrimidin-4-yl]benzonitrile on chiral prep-HPLC under the following conditions: column, CHIRALPAK IF-3, 0.46×10 cm, 3 um; mobile phase, MtBE (with 0.1% DEA) in MeOH, 90% isocratic in 30 min; detector, UV 254 nm.

Example 139: 2-[[(4S)-3,3-difluoro-1-[(2S)-2-hydroxypropanoyl]piperidin-4-yl]oxy]-5-[2-([4-[4-(oxetan-3-yl)piperazin-1-yl]phenyl]amino)pyrimidin-4-yl]benzonitrile: (100 mg, 40%, yellow solid) HPLC: 97.1% purity, RT=6.44 min. MS: m/z=620.2 [M+H]⁺. ¹H NMR (400 MHz, DMSO-d₆) δ 9.45 (s, 1H), 8.60-8.43 (m, 3H), 7.70-7.57 (m, 3H), 7.41 (d, J=5.2 Hz, 1H), 6.96-6.88 (m, 2H), 5.43-5.32 (m, 1H), 5.24 (d, J=6.9 Hz, 1H), 4.69-4.39 (m, 5H), 4.32-3.39 (m, 5H), 3.14-3.07 (m, 4H), 2.45-2.38 (m, 4H), 2.24-1.78 (m, 2H), 1.23 (d, J=4.9 Hz, 3H).

Example 140: 2-[[(4R)-3,3-difluoro-1-[(2S)-2-hydroxypropanoyl]piperidin-4-yl]oxy]-5-[2-([4-[4-(oxetan-3-yl)piperazin-1-yl]phenyl]amino)pyrimidin-4-yl]benzonitrile (99 mg, 39%, yellow solid) HPLC: 98.8% purity, RT=10.84 min. MS: m/z=664.1 [M+H]⁺. ¹H NMR (400 MHz, DMSO-d₆) δ 9.45 (s, 1H), 8.58-8.45 (m, 3H), 7.70-7.58 (m, 3H), 7.41 (d, J=5.2 Hz, 1H), 6.96-6.89 (m, 2H), 5.37 (br s, 1H), 5.26-5.19 (m, 1H), 4.62-4.44 (m, 5H), 4.28-3.39 (m, 5H), 3.14-3.07 (m, 4H), 2.45-2.37 (m, 4H), 2.24-1.82 (m, 2H), 1.22 (d, J=6.5 Hz, 3H).

### Example 141: 2-([33-difluoro-1-[(2R)-2-hydroxypropanoyl]piperidin-4-yl]oxy)-5-[2-([4-[4-(oxetan-3-yl)piperazin-1-yl]phenyl]amino)pyrimidin-4-yl]benzonitrile 141

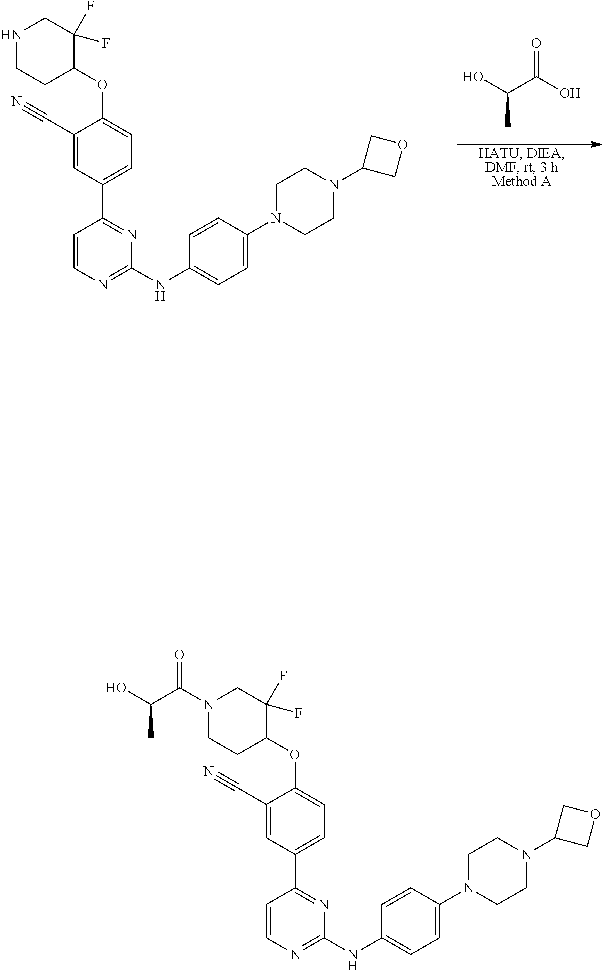

The title compound was prepared from 2-[(3,3-difluoropiperidin-4-yl)oxy]-5-[2-([4-[4-(oxetan-3-yl)piperazin-1-yl]phenyl]amino)pyrimidin-4-yl]benzonitrile and (2R)-2-hydroxypropanoic acid using Method A. The final product was purified by prep-HPLC under the following conditions: column, XBridge Prep C18 OBD Column, 150×19 mm, 5 um; mobile phase, acetonitrile in water (with 10 mmol/L NH₄HCO₃ and 0.1% NH₃.H₂O), 31% to 35% gradient in 8 min; detector, UV 254 nm. 2-([3,3-difluoro-1-[(2R)-2-hydroxypropanoyl]piperidin-4-yl]oxy)-5-[2-([4-[4-(oxetan-3-yl)piperazin-1-yl]phenyl]amino)pyrimidin-4-yl]benzonitrile was obtained as an yellow solid (25 mg, 24%). HPLC: 98.2% purity, RT=3.58 min. MS: m/z=620.2 [M+H]⁺. ¹H NMR (300 MHz, DMSO-d₆) δ 9.44 (s, 1H), 8.58-8.42 (m, 3H), 7.71-7.57 (m, 3H), 7.46-7.36 (m, 1H), 6.97-6.87 (m, 2H), 5.50-5.14 (m, 2H), 4.63-4.42 (m, 5H), 4.32-3.38 (m, 5H), 3.15-3.05 (m, 4H), 2.46-2.36 (m, 4H), 2.28-1.74 (m, 2H), 1.22 (d, J=6.4 Hz, 3H).

### Example 142 and 143: 2-[[(4S)-3,3-difluoro-1-[(2R)-2-hydroxypropanoyl]piperidin-4-yl]oxy]-5-[2-([4-[4-(oxetan-3-yl)piperazin-1-yl]phenyl]amino)pyrimidin-4-yl]benzonitrile & 2-[[(4R)-3,3-difluoro-1-[(2R)-2-hydroxypropanoyl]piperidin-4-yl]oxy]-5-[2-([4-[4-(oxetan-3-yl)piperazin-1-yl]phenyl]amino)pyrimidin-4-yl]benzonitrile 142 & 143

The two diastereomers were obtained by separation of 2-([3,3-difluoro-1-[(2R)-2-hydroxypropanoyl]piperidin-4-yl]oxy)-5-[2-([4-[4-(oxetan-3-yl)piperazin-1-yl]phenyl]amino)pyrimidin-4-yl]benzonitrile on chiral prep-HPLC under the following conditions: column, CHIRALPAKIG-3, 0.46×10 cm, 3 um; mobile phase, MtBE (with 0.1% DEA) in EtOH, 70% isocratic in 30 min; detector, UV 254 nm.

Example 142: 2-[[(4S)-3,3-difluoro-1-[(2R)-2-hydroxypropanoyl]piperidin-4-yl]oxy]-5-[2-([4-[4-(oxetan-3-yl)piperazin-1-yl]phenyl]amino)pyrimidin-4-yl]benzonitrile (61 mg, 25%, yellow solid) HPLC: 97.9% purity, RT=4.27 min. MS: m/z=619.3 [M+H]⁺. ¹H NMR (300 MHz, DMSO-d₆) δ 9.45 (s, 1H), 8.58-8.42 (m, 3H), 7.71-7.56 (m, 3H), 7.41 (d, J=5.2 Hz, 1H), 6.97-6.87 (m, 2H), 5.42-5.32 (m, 1H), 5.24 (d, J=6.9 Hz, 1H), 4.63-4.42 (m, 5H), 4.32-3.37 (m, 5H), 3.15-3.05 (m, 4H), 2.46-2.36 (m, 4H), 2.23-1.79 (m, 2H), 1.22 (d, J=6.4 Hz, 3H).

Example 143: 2-[[(4R)-3,3-difluoro-1-[(2R)-2-hydroxypropanoyl]piperidin-4-yl]oxy]-5-[2-([4-[4-(oxetan-3-yl)piperazin-1-yl]phenyl]amino)pyrimidin-4-yl]benzonitrile: (77 mg, 31%, yellow solid) HPLC: 99.8% purity, RT=4.29 min. MS: m/z=620.0 [M+H]⁺. ¹H NMR (300 MHz, DMSO-d₆) δ 9.45 (s, 1H), 8.58-8.42 (m, 3H), 7.71-7.57 (m, 3H), 7.41 (d, J=5.2 Hz, 1H), 6.97-6.87 (m, 2H), 5.40-5.34 (m, 1H), 5.22 (d, J=6.9 Hz, 1H), 4.63-4.43 (m, 5H), 4.35-3.37 (m, 5H), 3.16-3.06 (m, 4H), 2.46-2.36 (m, 5H), 2.24-1.78 (m, 2H), 1.22 (d, J=6.5 Hz, 3H)

### Example 144: 2-(3,3-Difluoro-piperidin-4-yloxy)-5-[2-(2-methoxy-1′-methyl-1′,2′,3′,4′,5′,6′-hexahydro-[3,4′]bipyridinyl-6-ylamino)-pyrimidin-4-yl]-benzonitrile 144

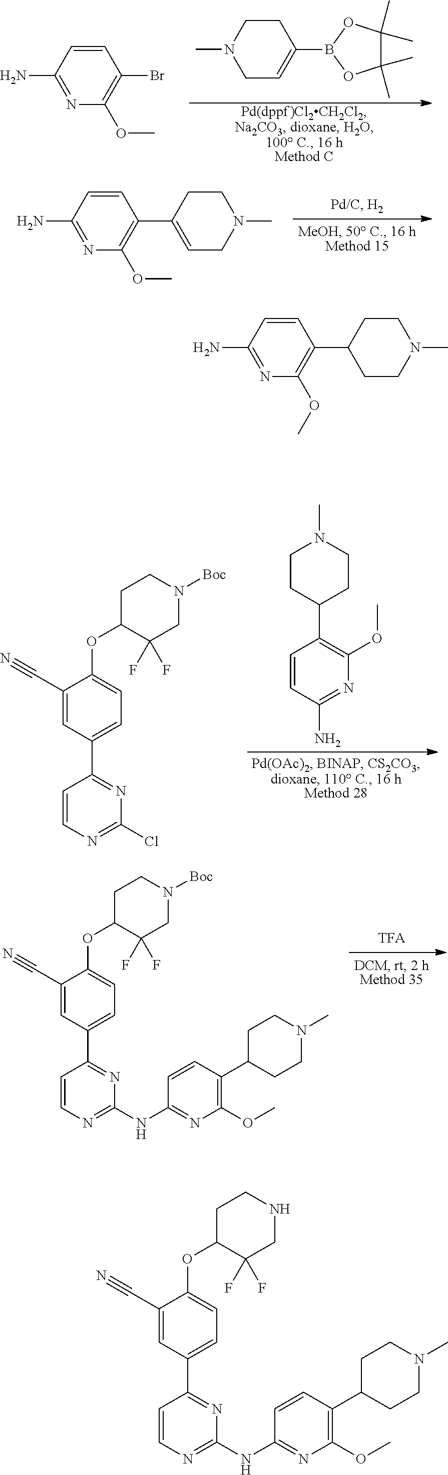

The title compound was prepared from 5-bromo-6-methoxypyridin-2-amine, 1-methyl-4-(4,4,5,5-tetramethyl-1,3,2-dioxaborolan-2-yl)-1,2,3,6-tetrahydropyridine, 1-methyl-4-(4,4,5,5-tetramethyl-1,3,2-dioxaborolan-2-yl)-1,2,3,6-tetrahydropyridine, tert-butyl 4-(4-(2-chloropyrimidin-4-yl)-2-cyanophenoxy)-3,3-difluoropiperidine-1-carboxylate and 2-hydroxypropanoic acid using Method C₁₋₁₅, 28, 35. HPLC: 95.9% purity, RT=3.70 min. MS: m/z=536.3 [M+H]⁺. ¹H NMR (300 MHz, DMSO-d₆) δ 9.52 (s, 1H), 8.66-8.57 (m, 2H), 8.57-8.46 (m, 1H), 7.85-7.75 (m, 1H), 7.70-7.53 (m, 3H), 5.28-5.18 (m, 1H), 3.89 (s, 3H), 3.21-3.07 (m, 1H), 3.0-2.82 (m, 4H), 2.73-2.62 (m, 2H), 2.19 (s, 3H), 2.13-1.78 (m, 5H), 1.72-1.54 (m, 4H).

### Example 145: 2-([3,3-difluoro-1-[(2R)-2-hydroxypropanoyl]piperidin-4-yl]oxy)-5-(2-[[4-(1-methylpiperidin-4-yl)phenyl]amino]pyrimidin-4-yl)benzonitrile 145

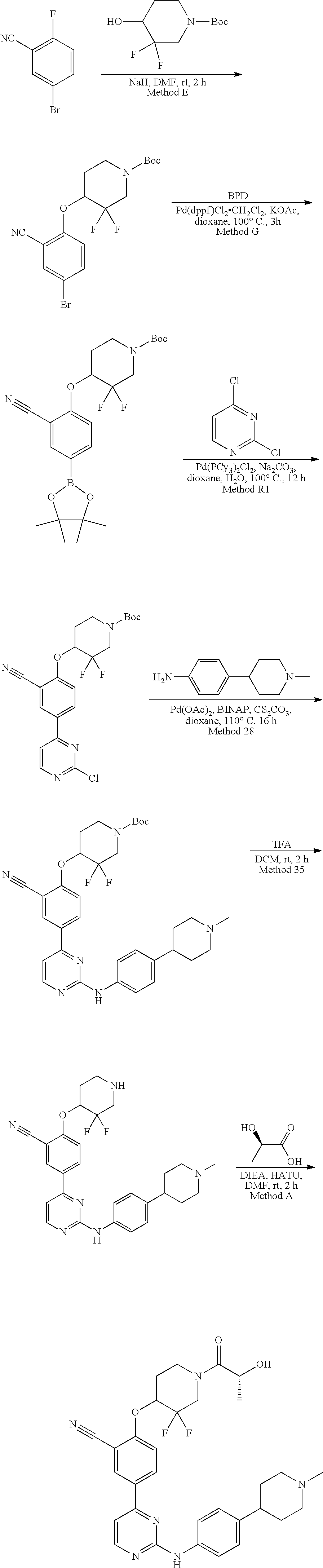

The title compound was prepared from 5-bromo-2-fluorobenzonitrile, tert-butyl 3,3-difluoro-4-hydroxypiperidine-1-carboxylate, BPD, 2,4-dichloropyrimidine, 4-(1-methyl piperidin-4-yl)benzenamine, and (R)-2-hydroxypropanoic acid using Method E, G, R1, 28, 35 and A. The final product was purified by prep-HPLC under the following conditions: column, Atlantis HILIC OBD Column, 150×19 mm, 5 um; mobile phase, acetonitrile in water (with 10 mmol/L NH₄HCO₃ and 0.1% NH₃.H₂O), 25% to 55% gradient in 8 min; detector, UV 254 nm. 2-([3,3-difluoro-1-[(2R)-2-hydroxypropanoyl]piperidin-4-yl]oxy)-5-(2-[[4-(1-methylpiperidin-4-yl)phenyl]amino]pyrimidin-4-yl)benzonitrile was obtained as an off-white solid (26 mg, 3.2% for 6 steps). HPLC: 92.4% purity, RT=3.10 min. MS: m/z=577.3 [M+H]⁺. ¹H NMR (300 MHz, DMSO-d₆) δ 9.63 (s, 1H), 8.61-8.44 (m, 3H), 7.75-7.62 (m, 3H), 7.51-7.41 (m, 1H), 7.23-7.13 (m, 2H), 5.42-5.35 (m, 1H), 5.29-5.19 (m, 1H), 4.56-4.45 (m, 1H), 4.30-3.49 (m, 4H), 2.91-2.81 (m, 2H), 2.48-2.32 (m, 1H), 2.21-2.16 (m, 4H), 2.03-1.89 (m, 3H), 1.78-1.56 (m, 4H), 1.22 (d, J=6.5 Hz, 3H).

### Example 146: 2-[[3,3-difluoro-1-(2-hydroxypropanoyl)piperidin-4-yl]oxy]-5-(2-[[6-methoxy-5-(1-methylpiperidin-4-yl)pyridin-2-yl]amino]pyrimidin-4-yl)benzonitrile 146

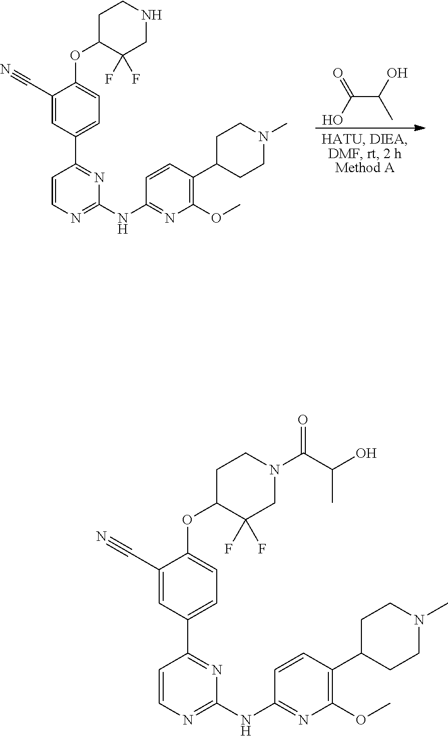

The title compound was prepared from 2-(3,3-Difluoro-piperidin-4-yloxy)-5-[2-(2-methoxy-1′-methyl-1′,2′,3′,4′,5′,6′-hexahydro-[3,4′]bipyridinyl-6-ylamino)-pyrimidin-4-yl]-benzonitrile and 2-hydroxypropanoic acid using Method A. The final product was purified by prep-HPLC under the following conditions: column, XBridge Prep OBD C18 Column, 150×19 mm, 5 um; mobile phase, acetonitrile in water (with 10 mmol/L NH₄HCO₃ and 0.1% NH₃.H₂O), 30% to 60% gradient in 8 min; detector, UV 254 nm. 2-[[3,3-difluoro-1-(2-hydroxypropanoyl)piperidin-4-yl]oxy]-5-(2-[[6-methoxy-5-(1-methylpiperidin-4-yl)pyridin-2-yl]amino]pyrimidin-4-yl)benzonitrile was obtained as off-white solid (16 mg, 1.2% for 5 steps). HPLC: 91.0% purity, RT=4.49 min. MS: m/z=608.4 [M+H]⁺. ¹H NMR (300 MHz, DMSO-de) δ 9.52 (s, 1H), 9.02-8.41 (m, 3H), 8.01-7.48 (m, 4H), 5.43-5.36 (m, 1H), 5.30-5.21 (m, 1H), 4.54-4.47 (m, 1H), 4.33-3.50 (m, 7H), 2.91-2.81 (m, 2H), 2.68-2.61 (m, 1H), 2.19 (s, 3H), 2.18-2.05 (m, 1H) 2.02-1.87 (m, 3H), 1.78-1.49 (m, 4H), 1.22 (d, J=6.4 Hz, 3H).

### Example 147: 2-([3,3-difluoro-1-[(2R)-2-hydroxypropanoyl]piperidin-4-yl]oxy)-5-(2-[[6-methoxy-5-(1-methylpiperidin-4-yl)pyridin-2-yl]amino]pyrimidin-4-yl)benzonitrile 147

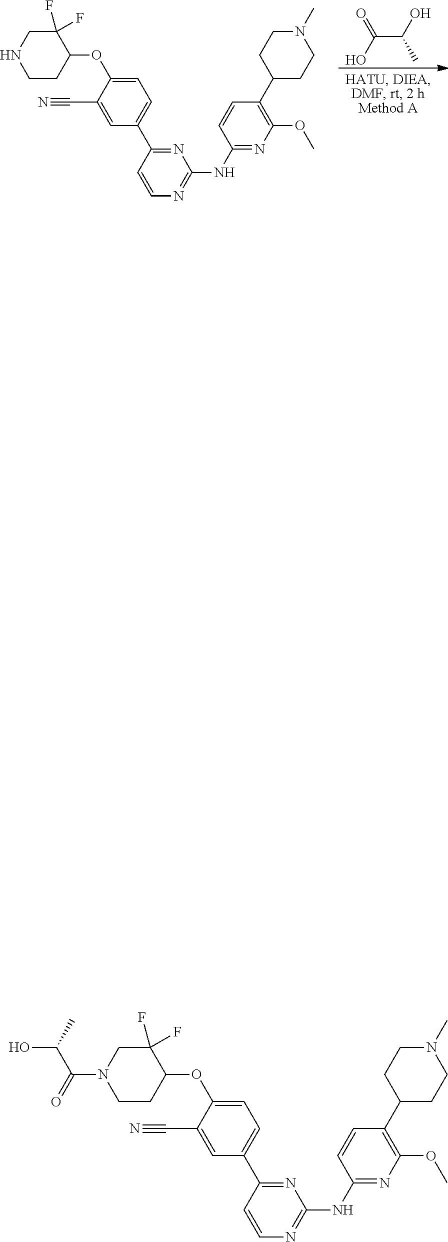

The title compound was prepared from 2-[(3,3-difluoropiperidin-4-yl)oxy]-5-(2-[[6-methoxy-5-(1-methylpiperidin-4-yl)pyridin-2-yl]amino]pyrimidin-4-yl)benzonitrile and (2R)-2-hydroxypropanoic acid using Method A. The final product was purified by prep-HPLC under the following conditions: column, XBridge Prep OBD C18 Column, 150×19 mm, 5 um; mobile phase, acetonitrile in water (with 10 mmol/L NH₄HCO₃ and 0.1% NH₃.H₂O), 30% to 60% gradient in 8 min; detector, UV 254 nm. 2-([3,3-difluoro-1-[(2R)-2-hydroxypropanoyl]piperidin-4-yl]oxy)-5-(2-[[6-methoxy-5-(1-methylpiperidin-4-yl)pyridin-2-yl]amino]pyrimidin-4-yl)benzonitrile was obtained as off-white solid (16 mg, 20%). HPLC: 90.2% purity, RT=4.51 min. MS: m/z=608.5 [M+H]⁺. ¹H NMR (300 MHz, DMSO-d₆) δ 9.52 (s, 1H), 8.77-8.48 (m, 3H), 7.89-7.48 (m, 4H), 5.43-5.36 (m, 1H), 5.30-5.21 (m, 1H), 4.63-4.43 (m, 1H), 4.30-3.53 (m, 7H), 2.91-2.81 (m, 2H), 2.68-2.61 (m, 1H), 2.18-2.00 (m, 4H), 2.01-1.87 (m, 3H), 1.72-1.57 (m, 4H), 1.22 (d, J=6.5 Hz, 3H).

### Example 148: 2-[[3,3-difluoro-1-(2-hydroxyacetyl)piperidin-4-yl]oxy]-5-(2-[[4-(1-methylpiperidin-4-yl)phenyl]amino]pyrimidin-4-yl)benzonitrile 148

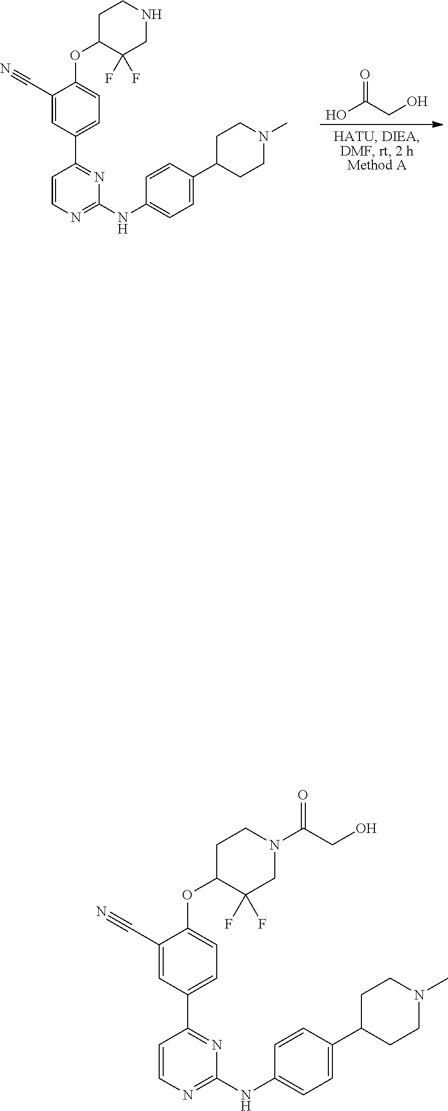

The title compound was prepared from 2-[(3,3-difluoropiperidin-4-yl)oxy]-5-(2-[[4-(l-methylpiperidin-4-yl)phenyl]amino]pyrimidin-4-yl)benzonitrile and 2-hydroxy acetic acid using Method A. The final product was purified by prep-HPLC under the following conditions: column, XBridge Prep OBD C18 Column, 150×19 mm, 5 um; mobile phase, acetonitrile in water (with 10 mmol/L NR₄HCO₃ and 0.1% NH₃.H₂O), 25% to 55% gradient in 8 min; detector, UV 254 nm. 2-[[3,3-difluoro-1-(2-hydroxyacetyl)piperidin-4-yl]oxy]-5-(2-[[4-(1-methylpiperidin-4-yl)phenyl]amino]pyrimidin-4-yl)benzonitrile was obtained as off-white solid (26 mg, 23%). HPLC: 97.1% purity, RT=2.97 min. MS: m/z=563.3 [M+H]⁺. ¹H NMR (300 MHz, DMSO-d₆) δ 9.63 (s, 1H), 8.61-8.44 (m, 3H), 7.75-7.60 (m, 3H), 7.47 (d, J=5.2 Hz, 1H), 7.23-7.13 (m, 2H), 5.45-5.31 (m, 1H), 4.95-4.84 (m, 1H), 4.28-3.40 (m, 6H), 2.91-2.81 (m, 2H), 2.49-2.31 (m, 1H), 2.19 (s, 3H), 2.18-1.81 (m, 4H), 1.79-1.56 (m, 4H).

### Example 149: 2-[[3,3-difluoro-1-(2-hydroxypropanoyl)piperidin-4-yl]oxy]-5-(2-[[4-(1-methylpiperidin-4-yl)phenyl]amino]pyrimidin-4-yl)benzonitrile 149

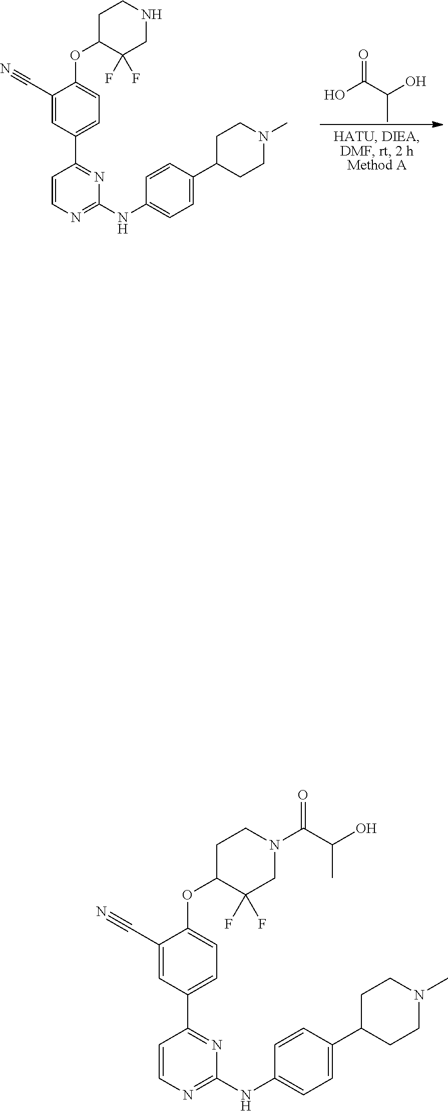

The title compound was prepared from 2-[(3,3-difluoropiperidin-4-yl)oxy]-5-(2-[[4-(1-methylpiperidin-4-yl)phenyl]amino]pyrimidin-4-yl)benzonitrile and 2-hydroxypropanoic acid using Method A. The final product was purified by prep-HPLC under the following conditions: column, XBridge Prep OBD C18 Column, 150×19 mm, 5 um; mobile phase, acetonitrile in water (with 10 mmol/L NR₄HCO₃ and 0.1% NH₃.H₂O), 25% to 55% gradient in 8 min; detector, UV 254 nm. 2-[[3,3-difluoro-1-(2-hydroxypropanoyl)piperidin-4-yl]oxy]-5-(2-[[4-(1-methylpiperidin-4-yl)phenyl]amino]pyrimidin-4-yl)benzonitrile was obtained as off-white solid (26 mg, 16%). HPLC: 94.7% purity, RT=3.11 min. MS: m/z=577.3 [M+H]⁺. ¹H NMR (300 MHz, DMSO-d₆) δ 9.61 (s, 1H), 8.59-8.42 (m, 3H), 7.73-7.61 (m, 3H), 7.49-7.41 (m, 1H), 7.21-7.11 (m, 2H), 5.37 (br s, 1H), 5.28-5.17 (m, 1H), 4.49 (br s, 1H), 4.34-3.40 (m, 4H), 2.90-2.79 (m, 2H), 2.46-2.31 (m, 1H), 2.17 (s, 3H), 2.15-1.87 (m, 4H), 1.78-1.55 (m, 4H), 1.21 (d, J=6.5 Hz, 3H).

### Example 150: 2-(3,3-Difluoro-piperidin-4-yloxy)-5-{2-[4-(1-oxetan-3-yl-piperidin-4-yl)-phenylamino]-pyrimidin-4-yl}-benzonitrile hydrochloride: 150

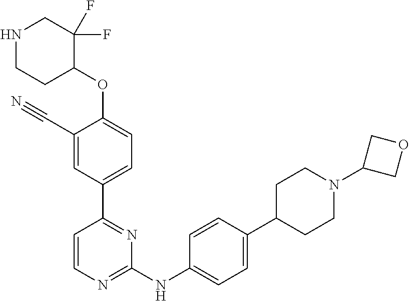 [SMILES: N#Cc1cc(-c2ccnc(Nc3ccc(C4CCN(C5COC5)CC4)cc3)n2)ccc1OC1CCNCC1(F)F]

The title compound was prepared according to the procedures described in example 116 by coupling 4-[4-(2-Amino-pyrimidin-4-yl)-2-cyano-phenoxy]-3,3-difluoro-piperidine-1-carboxylic acid tert-butyl ester with 4-(4-Bromo-phenyl)-1-oxetan-3-yl-piperidine followed by treating 4-(2-Cyano-4-{2-[4-(1-oxetan-3-yl-piperidin-4-yl)-phenylamino]-pyrimidin-4-yl}-phenoxy)-3,3-difluoro-piperidine-1-carboxylic acid tert-butyl ester with HCl in dioxane. MS: m/z=547.2 [M+H]⁺.

### Example 151: 5-{2-[4-(1-Oxetan-3-yl-piperidin-4-yl)-phenylamino]-pyrimidin-4-yl}-2-(tetrahydro-pyran-4-yloxy)-benzonitrile 151

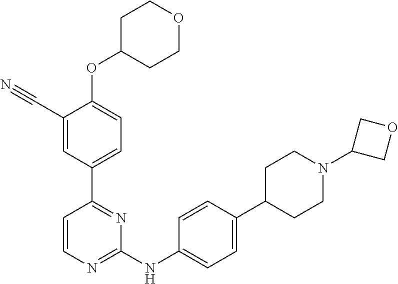 [SMILES: N#Cc1cc(-c2ccnc(Nc3ccc(C4CCN(C5COC5)CC4)cc3)n2)ccc1OC1CCOCC1]

The title compound was prepared according to the procedures described in example 116 using 5-(2-Amino-pyrimidin-4-yl)-2-(tetrahydro-pyran-4-yloxy)-benzonitrile, and 4-(4-Bromo-phenyl)-1-oxetan-3-yl-piperidine. MS: m/z=512.3 [M+H]⁺. 1H NMR (400 MHz, Chloroform-d) δ 8.47 (d, J=5.2 Hz, 1H), 8.32 (d, J=2.3 Hz, 1H), 8.25 (dd, J=8.9, 2.3 Hz, 1H), 7.61 (d, J=8.2 Hz, 2H), 7.26 (d, J=8.3 Hz, 2H), 7.08 (dd, J=15.9, 7.1 Hz, 2H), 4.83-4.65 (m, 5H), 4.05 (ddd, J=11.2, 7.1, 3.6 Hz, 2H), 3.67 (ddd, J=11.3, 7.2, 3.6 Hz, 2H), 2.95 (d, J=14.2 Hz, 3H), 2.63-2.47 (m, 1H), 2.18-1.80 (m, 11H).

### Example 152.2-[3,3-Difluoro-1-((S)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile 152

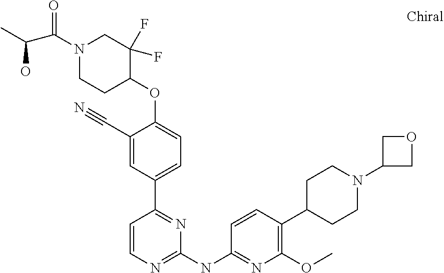 [SMILES: COc1nc(Nc2nccc(-c3ccc(OC4CCN(C(=O)[C@H](C)O)CC4(F)F)c(C#N)c3)n2)ccc1C1CCN(C2COC2)CC1]

The title compound (26.5 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), (S)-2-Hydroxy-propionic acid (15 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 43% yield, m/z: 650 (M+H). ¹H NMR (DMSO-d6): 9.47 (1H), 8.62 (2H), 8.52 (1H), 7.81 (1H), 7.65 (1H), 7.59 (1H), 5.20 (1H), 4.56 (2H), 4.45 (2H), 3.90 (3H), 3.42 (1H), 3.18 (1H), 2.91 (2H), 2.81 (1H), 2.73 (1H), 2.07 (1H), 1.86 (3H), 1.70 (3H).

### Example 153.2-[1-((S)-2,3-Dihydroxy-propionyl)-3,3-difluoro-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile 153

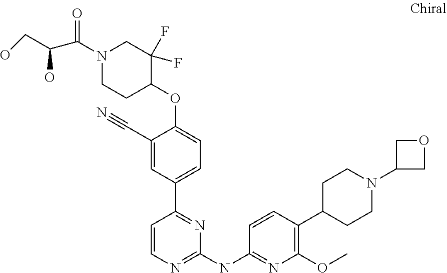 [SMILES: COc1nc(Nc2nccc(-c3ccc(OC4CCN(C(=O)[C@@H](O)CO)CC4(F)F)c(C#N)c3)n2)ccc1C1CCN(C2COC2)CC1]

The title compound (10.1 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), (S)-2,3-Dihydroxy-propionic acid (18 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 17% yield, m/z: 665 (M+H). ¹H NMR (DMSO-d6): 9.47 (1H), 8.62 (2H), 8.52 (1H), 7.81 (1H), 7.65 (1H), 7.59 (1H), 5.20 (1H), 4.56 (2H), 4.45 (2H), 3.90 (3H), 3.42 (1H), 3.18 (1H), 2.91 (2H), 2.81 (1H), 2.73 (1H), 2.07 (1H), 1.86 (5H).

### Example 154.2-[3,3-Difluoro-1-(2-hydroxy-acetyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile 154

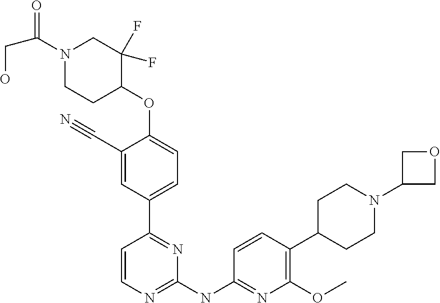 [SMILES: COc1nc(Nc2nccc(-c3ccc(OC4CCN(C(=O)CO)CC4(F)F)c(C#N)c3)n2)ccc1C1CCN(C2COC2)CC1]

The title compound (22.1 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), Hydroxy-acetic acid (13 mg), 0-(7-Azabenzotriazol-1-yl)-N,N,N′,N′-tetramethyluronium hexaflurophosphate (HATU) (57 mg) and Ethyl-diisopropyl-amine (55 mg) using Method A in 40% yield, m/z: 636 (M+H). ¹H NMR (DMSO-d6): 9.47 (1H), 8.62 (2H), 8.52 (1H), 7.81 (1H), 7.65 (1H), 7.59 (1H), 5.37 (1H), 4.56 (2H), 4.45 (2H), 4.21 (1H), 4.11 (1H), 3.90 (3H), 3.42 (1H), 3.18 (1H), 2.91 (2H), 2.81 (1H), 2.73 (1H), 2.07 (1H), 1.82 (2H), 1.72 (4H).

### Example 155: 2-(3,3-Difluoro-piperidin-4-yloxy)-5-{2-[4-(1-oxetan-3-yl-pyrrolidin-3-yl)-phenylamino]-pyrimidin-4-yl}-benzonitrile hydrochloride 155

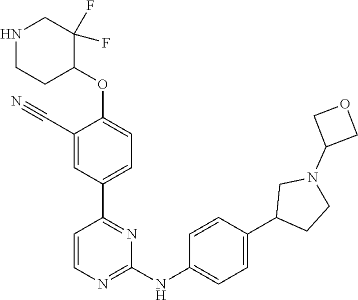 [SMILES: N#Cc1cc(-c2ccnc(Nc3ccc(C4CCN(C5COC5)C4)cc3)n2)ccc1OC1CCNCC1(F)F]

The title compound was prepared according to the procedures described in example 116 by coupling 4-(2-Cyano-4-{2-[4-(1-oxetan-3-yl-pyrrolidin-3-yl)-phenylamino]-pyrimidin-4-yl}-phenoxy)-3,3-difluoro-piperidine-1-carboxylic acid tert-butyl ester with 3-(4-Bromo-phenyl)-1-oxetan-3-yl-pyrrolidine followed by treatment of 4-(2-Cyano-4-{2-[4-(1-oxetan-3-yl-pyrrolidin-3-yl)-phenylamino]-pyrimidin-4-yl}-phenoxy)-3,3-difluoro-piperidine-1-carboxylic acid tert-butyl ester with HCl in dioxane. MS: m/z=533.3 [M+H]⁺.

### Example 156. 2-(3,3-Difluoro-piperidin-4-yloxy)-5-(2-{3-[1-(2-hydroxy-1-hydroxymethyl-ethyl)-piperidin-4-yl]-phenylamino}-pyrimidin-4-yl)-benzonitrile 156

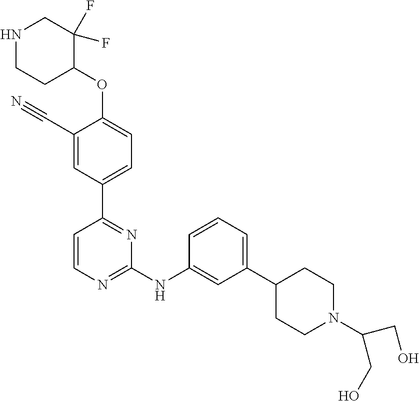 [SMILES: N#Cc1cc(-c2ccnc(Nc3cccc(C4CCN(C(CO)CO)CC4)c3)n2)ccc1OC1CCNCC1(F)F]

A mixture of 4-(2-Cyano-4-{2-[3-(1-oxetan-3-yl-piperidin-4-yl)-phenylamino]-pyrimidin-4-yl}-phenoxy)-3,3-difluoro-piperidine-1-carboxylic acid tert-butyl ester (680.00 mg; 1.05 mmol; 1.00 eq.) in Dioxane was added 4M HCl in dioxane (3 mL). The reaction mixture was stirred at room temperature for 2 hours. LCMS showed the reaction was complete, the product was observed. The solvent was removed and the product was suspended in DMC (50 mL). The solution was adjusted pH to 9-10 with addition of aqueous K2CO3. The DCM layer was combined and concentrated. The product was purified through reverse phase HPLC with 30% MeOH in Water containing 0.1% NH4OH to 100% MeOH containing 0.1% NH4OH in 22 minutes at the flow rate of 40 mL/minute to provide the product (300.00 mg; 0.53 mmol) in 51%. m/z: 565 (M+H). ¹H NMR (DMSO-d6): 9.62 (1H), 8.57 (1H), 8.49 (1H), 7.83 (1H), 7.62 (1H), 7.53 (1H), 7.49 (1H), 7.23 (1H), 6.86 (1H), 5.26 (1H), 3.59 (1H), 3.41 (1H), 3.18 (1H), 2.89 (3H), 2.03 (2H), 1.78 (2H), 1.68 (2H).

### Example 157: 2-[3,3-Difluoro-1-((S)-2-hydroxy-propionyl)-piperidin-4-yloxy]-5-(2-{3-[1-(2-hydroxy-1-hydroxymethyl-ethyl)-piperidin-4-yl]-phenylamino}-pyrimidin-4-yl)-benzonitrile 157

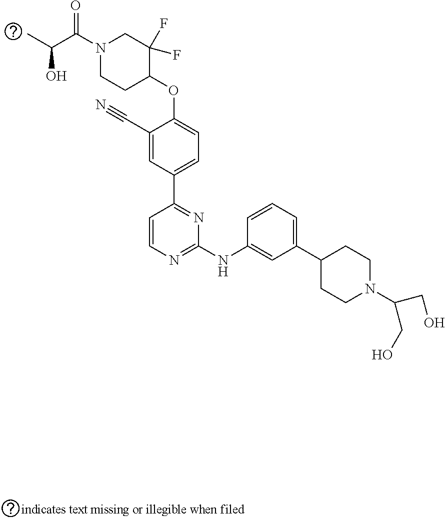 [SMILES: C[C@H](O)C(=O)N1CCC(Oc2ccc(-c3ccnc(Nc4cccc(C5CCN(C(CO)CO)CC5)c4)n3)cc2C#N)C(F)(F)C1]

The title compound (18.6 mg) was synthesized using 2-(3,3-Difluoro-piperidin-4-yloxy)-5-{2-[3-(1-oxetan-3-yl-piperidin-4-yl)-phenylamino]-pyrimidin-4-yl}-benzonitrile (50 mg), (S)-2,3-Dihydroxy-propionic acid (18 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 33% yield, m/z: 637 (M+H). ¹H NMR (DMSO-d6): 9.62 (1H), 8.57 (1H), 8.51 (1H), 7.73 (1H), 7.62 (1H), 7.53 (1H), 7.49 (1H), 7.23 (1H), 6.86 (1H), 5.55 (1H), 5.39 (1H), 5.26 (1H), 3.59 (1H), 3.41 (1H), 3.18 (1H), 2.89 (3H), 2.03 (2H), 1.78 (2H), 1.68 (2H), 1.24 (3H).

### Example 158.2-[3,3-Difluoro-1-((R)-2-hydroxy-propionyl)-piperidin-4-yloxy]-5-(2-{3-[1-(2-hydroxy-1-hydroxymethyl-ethyl)-piperidin-4-yl]-phenylamino}-pyrimidin-4-yl)-benzonitrile 158

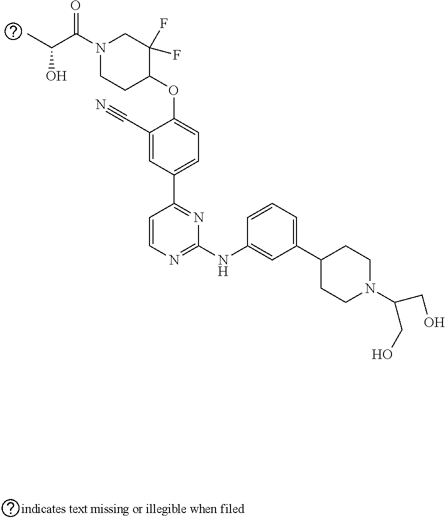 [SMILES: C[C@@H](O)C(=O)N1CCC(Oc2ccc(-c3ccnc(Nc4cccc(C5CCN(C(CO)CO)CC5)c4)n3)cc2C#N)C(F)(F)C1]

The title compound (12.3 mg) was synthesized using 2-(3,3-Difluoro-piperidin-4-yloxy)-5-{2-[3-(1-oxetan-3-yl-piperidin-4-yl)-phenylamino]-pyrimidin-4-yl}-benzonitrile (60 mg), (R)-2,3-Dihydroxy-propionic acid (19 mg), O-(7-Azabenzotriazol-1-yl)-N,N,N′,N′-tetramethyluronium hexaflurophosphate (HATU) (90 mg) and Ethyl-diisopropyl-amine (68 mg) using Method A in 33% yield, m/z: 637 (M+H). ¹H NMR (DMSO-d6): 9.62 (1H), 8.57 (1H), 8.51 (1H), 7.73 (1H), 7.62 (1H), 7.53 (1H), 7.49 (1H), 7.23 (1H), 6.86 (1H), 5.55 (1H), 5.39 (1H), 5.26 (1H), 3.59 (1H), 3.41 (1H), 3.18 (1H), 2.89 (3H), 2.03 (2H), 1.78 (2H), 1.68 (2H), 1.24 (3H).

### Example 159:5-[2-([4-[4-(oxetan-3-yl)piperazin-1-yl]phenyl]amino)pyrimidin-4-yl]-2-(oxetan-3-yloxy)benzonitrile

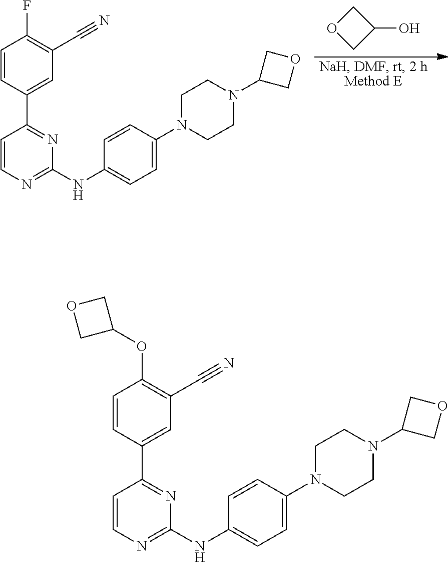

The title compound was prepared from 2-fluoro-5-[2-([4-[4-(oxetan-3-yl)piperazin-1-yl]phenyl]amino)pyrimidin-4-yl]benzonitrile and oxetan-3-ol using Method E. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 25% to 47% gradient in 8 min; detector, UV 254 nm. 5-[2-([4-[4-(oxetan-3-yl)piperazin-1-yl]phenyl]amino)pyrimidin-4-yl]-2-(oxolan-3-yloxy)benzonitrile was obtained as a light yellow solid (28 mg, 28%). HPLC: 99.2% purity, RT=4.20 min. MS: m/z=530.1 [M+H]⁺. ¹H NMR (400 MHz, DMSO-d₆, ppm) δ 9.42 (s, 1H), 8.57-8.36 (m, 3H), 7.66-7.57 (m, 2H), 7.41-7.35 (m, 1H), 7.10-7.03 (m, 1H), 6.96-6.87 (m, 2H), 5.59-5.49 (m, 1H), 5.05-4.96 (m, 2H), 4.67-4.53 (m, 4H), 4.52-4.44 (m, 2H), 3.51-3.40 (m, 1H), 3.14-3.07 (m, 4H), 2.45-2.38 (m, 4H).

### Example 160: 5-[2-([6-methoxy-5-[4-(oxetan-3-yl)piperazin-1-yl]pyridin-2-yl]amino)pyrimidin-4-yl]-2-(oxetan-3-yloxy)benzonitrile

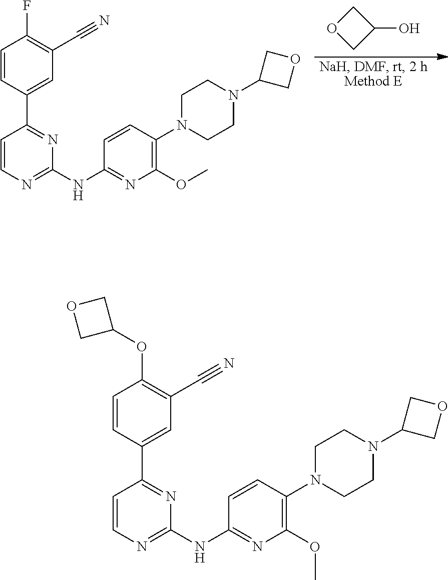

The title compound was prepared from 2-fluoro-5-[2-([6-methoxy-5-[4-(oxetan-3-yl)piperazin-1-yl]pyridin-2-yl]amino)pyrimidin-4-yl]benzonitrile and oxelan-3-ol using Method E. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 27% to 50% gradient in 8 min; detector, UV 254 nm. 5-[2-([6-methoxy-5-[4-(oxetan-3-yl)piperazin-1-yl]pyridin-2-yl]amino)pyrimidin-4-yl]-2-(oxetan-3-yloxy)benzonitrile was obtained as a light yellow solid (25 mg, 25%). HPLC: 98.2% purity, RT=4.07 min. MS: m/z=516.1[M+H]⁺. ¹H NMR (300 MHz, Chloroform-d, ppm) δ 8.56-8.48 (m, 1H), 8.40-8.33 (m, 1H), 8.28-8.18 (m, 1H), 7.91-7.82 (m, 1H), 7.65 (s, 1H), 7.31-7.21 (m, 1H), 7.13-7.05 (m, 1H), 6.70-6.61 (m, 1H), 5.46-5.32 (m, 1H), 5.10-4.99 (m, 2H), 4.94-4.83 (m, 2H), 4.75-4.66 (m, 4H), 3.97 (s, 3H), 3.66-3.59 (m, 1H), 3.17-3.10 (m, 4H), 2.62-2.55 (m, 4H).

### Example 161: 5-[2-([4-[4-(oxetan-3-yl)piperazin-1-yl]phenyl]amino)pyrimidin-4-yl]-2-(oxolan-3-yloxy)benzonitrile

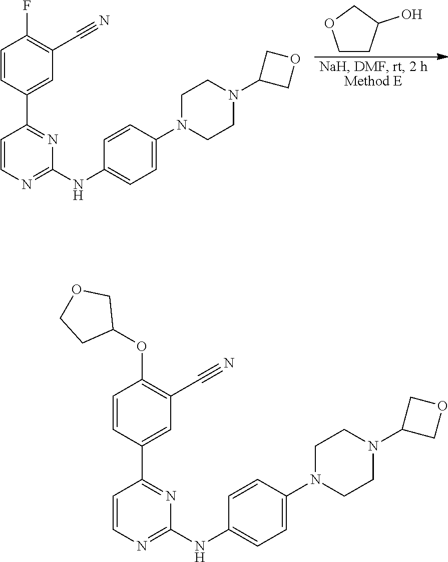

The title compound was prepared from 2-fluoro-5-[2-([4-[4-(oxetan-3-yl)piperazin-1-yl]phenyl]amino)pyrimidin-4-yl]benzonitrile and oxolan-3-ol using Method E. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 30% to 60% gradient in 8 min; detector, UV 254 nm. 5-[2-([4-[4-(oxetan-3-yl)piperazin-1-yl]phenyl]amino)pyrimidin-4-yl]-2-(oxolan-3-yloxy)benzonitrile was obtained as a light yellow solid (26 mg, 19%). HPLC: 98.8% purity, RT=4.34 min. MS: m/z=499.1 [M+H]⁺. ¹H NMR (400 MHz, DMSO-d₆, ppm) δ 9.41 (s, 1H), 8.54-8.40 (m, 3H), 7.65-7.58 (m, 2H), 7.46-7.35 (m, 2H), 6.98-6.89 (m, 2H), 5.36-5.30 (m, 1H), 4.62-4.53 (m, 2H), 4.52-4.44 (m, 2H), 4.00-3.85 (m, 3H), 3.85-3.75 (m, 1H), 3.48-3.43 (m, 1H), 3.13-3.08 (m, 4H), 2.44-2.39 (m, 4H), 2.39-2.27 (m, 1H), 2.11-2.01 (m, 1H).

### Example 162: 5-[2-([6-methoxy-5-[4-(oxetan-3-yl)piperazin-1-yl]pyridin-2-yl]amino)pyrimidin-4-yl]-2-(oxolan-3-yloxy)benzonitrile

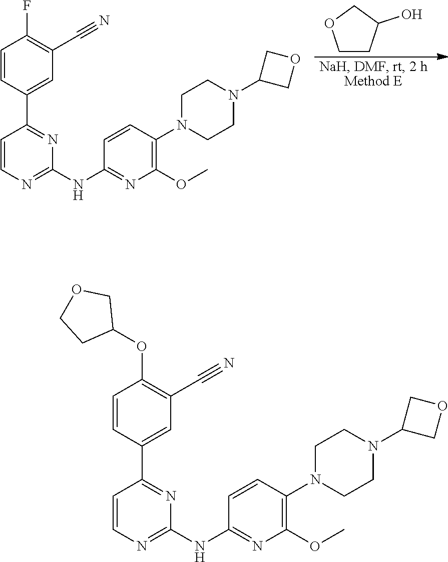

The title compound was prepared from 2-fluoro-5-[2-([6-methoxy-5-[4-(oxetan-3-yl)piperazin-1-yl]pyridin-2-yl]amino)pyrimidin-4-yl]benzonitrile and oxolan-3-ol using Method E. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 25% to 55% gradient in 8 min; detector, UV 254 nm. 5-[2-([6-methoxy-5-[4-(oxetan-3-yl)piperazin-1-yl]pyridin-2-yl]amino)pyrimidin-4-yl]-2-(oxolan-3-yloxy)benzonitrile was obtained as a light yellow solid (24 mg, 24%). HPLC: 99.2% purity, RT=4.20 min. MS: m/z=530.1 [M+H]⁺. ¹H NMR (300 MHz, Chloroform-d, ppm) δ 8.56-8.47 (m, 1H), 8.38-8.21 (m, 2H), 7.92-7.83 (m, 1H), 7.65 (s, 1H), 7.31-7.22 (m, 1H), 7.14-6.97 (m, 2H), 5.16-5.06 (m, 1H), 4.77-4.66 (m, 4H), 4.18-3.98 (m, 4H), 3.97 (s, 3H), 3.65-3.58 (m, 1H), 3.16-3.09 (m, 4H), 2.61-2.54 (m, 4H), 2.41-2.23 (m, 2H).

### Example 163: 5-{2-[4-(4-Methyl-piperazin-1-yl)-phenylamino]-pyrimidin-4-yl}-2-(tetrahydro-pyran-4-yloxy)-benzonitrile

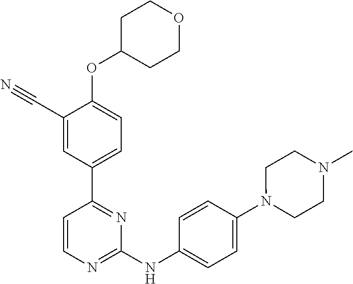 [SMILES: CN1CCN(c2ccc(Nc3nccc(-c4ccc(OC5CCOCC5)c(C#N)c4)n3)cc2)CC1]

A mixture of 5-(2-Chloro-pyrimidin-4-yl)-2-(tetrahydro-pyran-4-yloxy)-benzonitrile (200.00 mg; 0.63 mmol; 1.00 eq.), 4-(4-Methyl-piperazin-1-yl)-phenylamine (145.38 mg; 0.76 mmol; 1.20 eq.), 4,5-Bis-diphenylphosphanyl-9,9-dimethyl-9H-xanthene (0.12 ml; 0.19 mmol; 0.30 eq.), and Cs2CO3 (434.48 mg; 1.27 mmol; 2.00 eq.) in dioxane in a microwave vial was purged with argon for 3 min. Then Pd2(dba)₃CHCl3 (138.03 mg; 0.13 mmol; 0.20 eq.) was added. The reaction mixture was heated at 100° C. for overnight. Filtered and the solvent was removed. The residue was dissolved in EtOAc and purified on silica gel (Hex: EtOAc from 50:50 to 0:100, then, MeOH in EtOAc from 0% to 15%) to provide 5-{2-[4-(4-Methyl-piperazin-1-yl)-phenylamino]-pyrimidin-4-yl}-2-(tetrahydro-pyran-4-yloxy)-benzonitrile (49.10 mg; 0.10 mmol) in 16% yield. M/Z: 471 (M+H). ¹H NMR (DMSO-d6): 9.40 (1H), 8.50 (2H), 8.41 (1H), 7.63 (2H), 7.51 (1H), 7.38 (1H), 6.93 (2H), 4.93 (1H), 3.88 92H0, 3.56 (2H), 3.09 (4H), 2.27 (3H0, 2.06 (2H), 1.86 (2H).

### Example 164: 5-(2-[[5-(4-methylpiperazin-1-yl)pyridin-2-yl]amino]pyrimidin-4-yl)-2-(oxan-4-yloxy)benzonitrile

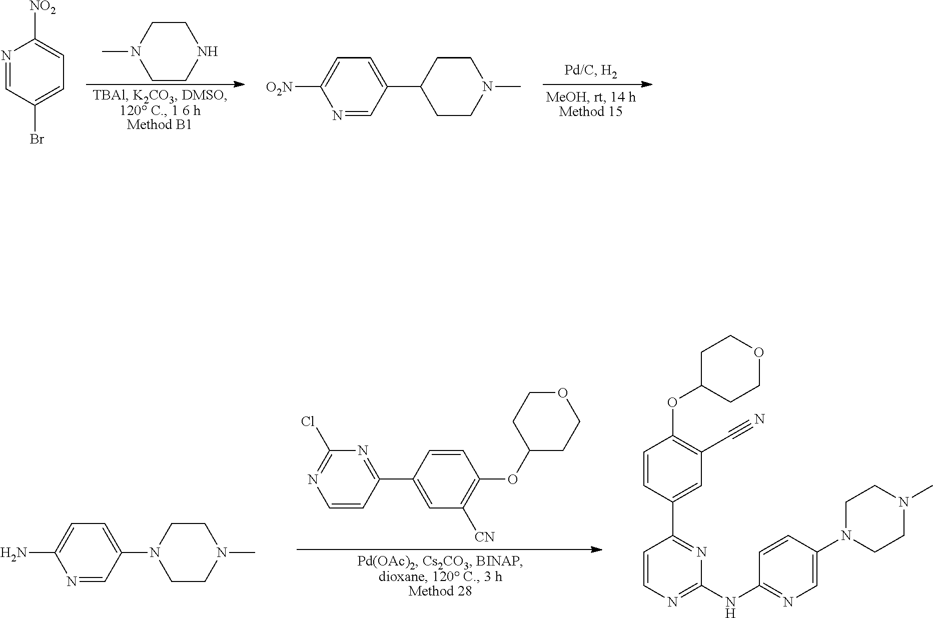

### Method B1

1-methyl-4-(6-nitropyridin-3-yl)piperazine: To a solution of 5-bromo-2-nitropyridine (475 mg, 2.34 mmol) in DMSO (2.5 mL) was added potassium carbonate (651 mg, 4.71 mmol), 1-methylpiperazine (343 mg, 3.43 mmol) and TBAI (9 mg, 0.02 mmol) at room temperature. The resulting mixture was stirred for 16 h at 120° C. When the reaction was done, the reaction mixture was diluted with H₂O (30 mL) and extracted with dichloromethane (50 mL×3). The organic phases were combined, washed with brine and dried over Na₂SO₄. The solvent was removed under reduced pressure and the residue was purified by flash chromatography eluting with MeOH in EtOAc (0% to 50% gradient) to yield 1-methyl-4-(6-nitropyridin-3-yl)piperazine as a yellow solid (433 mg, 83%). MS: m/z=222.9 [M+H]⁺.

5-(2-[[5-(4-methylpiperazin-1-yl)pyridin-2-yl]amino]pyrimidin-4-yl)-2-(oxan-4-yloxy)benzonitrile: The title compound was prepared using Methods 15 and 28. The final product was purified by prep-HPLC under the following conditions: column, XBridge Shield RP18 OBD Column, 150×19 mm, 5 um; mobile phase, acetonitrile in water (with 0.05% NH₃.H₂O), 31% to 53% gradient in 8 min; detector, UV 254 nm. 5-(2-[[5-(4-methylpiperazin-1-yl)pyridin-2-yl]amino]pyrimidin-4-yl)-2-(oxan-4-yloxy)benzonitrile was obtained as a yellow solid (25 mg, 21% for 2 steps). HPLC: 99.7% purity, RT=1.17 min. MS: m/z=472.2 [M+H]⁺. ¹H NMR (300 MHz, DMSO-d₆) δ 9.58 (s, 1H), 8.59-8.51 (m, 2H), 8.51-8.41 (m, 1H), 8.15-8.05 (m, 1H), 8.05-7.97 (m, 1H), 7.59-7.39 (m, 3H), 4.99-4.88 (m, 1H), 3.94-3.81 (m, 2H), 3.63-3.49 (m, 2H), 3.18-3.08 (m, 4H), 2.49-2.43 (m, 4H), 2.23 (s, 3H), 2.10-1.99 (m, 2H), 1.76-1.63 (m, 2H).

### Example 165: 5-{2-[5-(4-Oxetan-3-yl-piperazin-1-yl)-pyridin-2-ylamino]-pyrimidin-4-yl}-2-(tetrahydro-pyran-4-yloxy)-benzonitrile

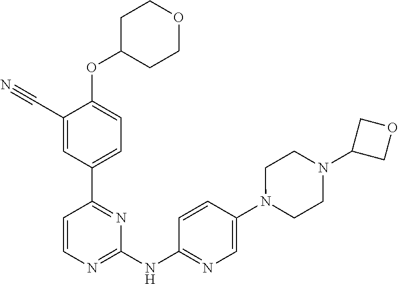 [SMILES: N#Cc1cc(-c2ccnc(Nc3ccc(N4CCN(C5COC5)CC4)cn3)n2)ccc1OC1CCOCC1]

The title compound was prepared from 5-(2-Chloro-pyrimidin-4-yl)-2-(tetrahydro-pyran-4-yloxy)-benzonitrile (150.00 mg; 0.48 mmol; 1.00 eq.), 5-(4-Oxetan-3-yl-piperazin-1-yl)-pyridin-2-ylamine (111.30 mg; 0.48 mmol; 1.00 eq.), BINAP (147.90 mg; 0.24 mmol; 0.50 eq.), Cs2CO3 (464.35 mg; 1.43 mmol; 3.00 eq.) in N,N-Dimethyl-formamide (15.00 ml) and Pd(OAc)₂ (53.33 mg; 0.24 mmol; 0.50 eq.) using the Method 28. HPLC: 94% purity; MS: m/z=546.3 [M+H]⁺.

### Example 166: 5-[2-([6-methoxy-5-[4-(2-methoxyethyl)piperazin-1-yl]piperidin-2-yl]amino)-1,3-diazinan-4-yl]-2-(oxan-4-yloxy)cyclohexane-1-carbonitrile

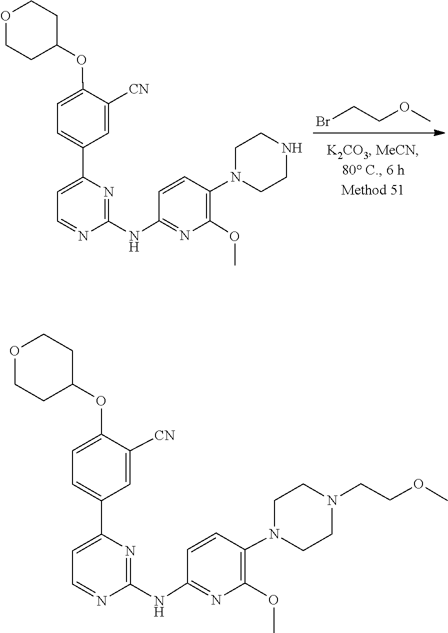

The title compound was prepared from 5-(2-(6-methoxy-5-(piperazin-1-yl)pyridin-2-ylamino)pyrimidin-4-yl)-2-(tetrahydro-2H-pyran-4-yloxy)benzonitrile and 1-bromo-2-methoxyethane using Method 51. The final product was purified by prep-HPLC under the following conditions: column, XBridge Prep OBD C18 Column, 150×19 mm, 5 um; mobile phase, MeCN in water (with 10 mmol/L NH₄HCO₃ and 0.1% NH₃.H₂O), 35% to 65% gradient in 8 min; detector, UV 254 nm. 5-[2-([6-methoxy-5-[4-(2-methoxyethyl)piperazin-1-yl]piperidin-2-yl]amino)-1,3-diazinan-4-yl]-2-(oxan-4-yloxy)cyclohexane-1-carbonitrile was obtained as an yellow solid (28 mg, 26%). HPLC: 99.8% purity, RT=4.88 min. MS: m/z=546.3 [M+H]⁺. ¹H NMR (300 MHz, DMSO-d6, ppm) δ 9.36 (s, 1H), 8.59-8.52 (m, 2H), 8.49-8.39 (m, 1H), 7.73-7.69 (m, 1H), 7.57-7.47 (m, 2H), 7.28-7.21 (m, 1H), 5.00-4.85 (m, 1H), 3.93-3.78 (m, 5H), 3.59-3.49 (m, 2H), 3.48-3.41 (m, 2H), 3.25 (s, 3H), 3.00-2.86 (m, 4H), 2.58-2.51 (m, 6H), 2.08-1.99 (m, 2H), 1.74-1.62 (m, 2H).

### Example 167: 5-[2-([5-[4-(2-hydroxyethyl)piperazin-1-yl]-6-methoxypyridin-2-yl]amino)pyrimidin-4-yl]-2-(oxan-4-yloxy)benzonitrile

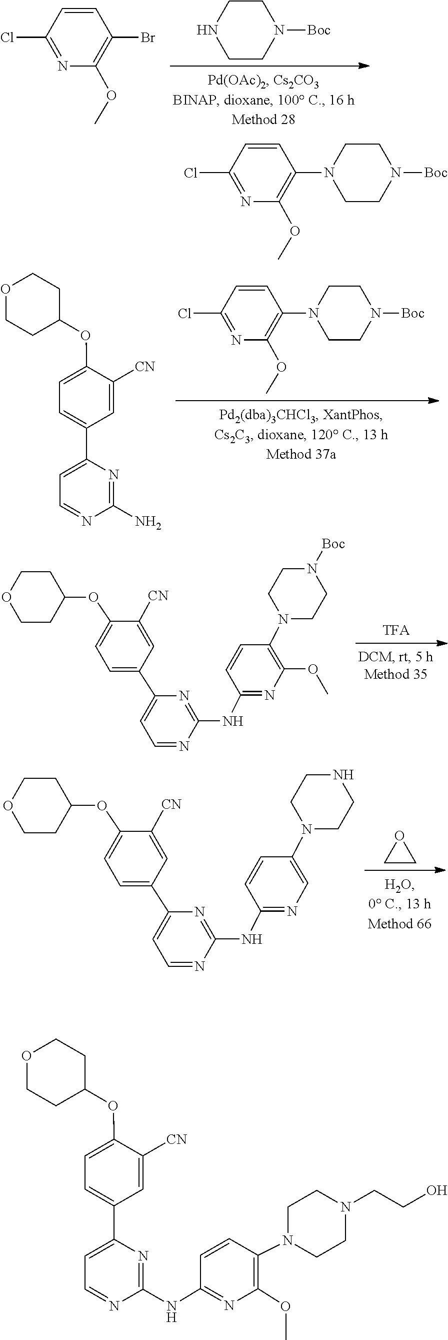

5-(2-[[6-methoxy-5-(piperazin-1-yl)pyridin-2-yl]amino]pyrimidin-4-yl)-2-(oxan-4-yloxy)benzonitrile: The title compound was prepared from 3-bromo-6-chloro-2-methoxypyridine, tert-butyl piperazine-1-carboxylate and 5-(2-aminopyrimidin-4-yl)-2-(tetrahydro-2H-pyran-4-yloxy)benzonitrile using Methods 28, 37a and 35 to yield 5-(2-[[6-methoxy-5-(piperazin-1-yl)pyridin-2-yl]amino]pyrimidin-4-yl)-2-(oxan-4-yloxy)benzonitrile was obtained as an yellow solid (603 mg, 69% for 2 steps). MS: m/z=488.0 [M+H]⁺.

### Method 66

5-[2-([5-[4-(2-hydroxyethyl)piperazin-1-yl]-6-methoxypyridin-2-yl]amino)pyrimidin-4-yl]-2-(oxan-4-yloxy)benzonitrile: At 0° C., to a solution of 5-(2-[[6-methoxy-5-(piperazin-1-yl)pyridin-2-yl]amino]pyrimidin-4-yl)-2-(oxan-4-yloxy)benzonitrile (140 mg, 0.286 mmol) in H₂O (60 mL) was added oxirane (31.80 mg, 0.721 mmol,) under nitrogen atmosphere. The resulting mixture was stirred for 13 h at 0° C. After the reaction was done, the reaction mixture was concentrated under reduced pressure and the residue was purified by prep-HPLC under the following conditions: column, XBridge Prep C18 OBD Column, 150×19 mm, 5 um; mobile phase, acetonitrile in water (with 10 mmol/L NH₄HCO₃), 24% to 51% gradient in 7 min; detector, UV 254 nm. 5-[2-([5-[4-(2-hydroxyethyl)piperazin-1-yl]-6-methoxypyridin-2-yl]amino)pyrimidin-4-yl]-2-(oxan-4-yloxy)benzonitrile was obtained as an yellow solid (37 mg, 11%). HPLC: 99.9% purity, RT=4.32 min. MS: m/z=532.2 [M+H]⁺. ¹H NMR (300 MHz, DMSO-d6, ppm) δ9.34 (s, 1H), 8.59-8.52 (m, 2H), 8.51-8.41 (m, 1H), 7.76-7.67 (m, 1H), 7.58-7.46 (m, 2H), 7.28-7.20 (m, 1H), 4.99-4.88 (m, 1H), 4.44-4.34 (m, 1H), 3.95-3.77 (m, 5H), 3.61-3.45 (m, 4H), 2.96-2.90 (m, 4H), 2.57-2.51 (m, 4H), 2.47-2.37 (m, 2H), 2.09-1.98 (m, 2H), 1.76-1.59 (m, 2H).

### Example 168: 5-(2-[[5-(4-acetylpiperazin-1-yl)-6-methoxypyridin-2-yl]amino]pyrimidin-4-yl)-2-(oxan-4-yloxy)benzonitrile

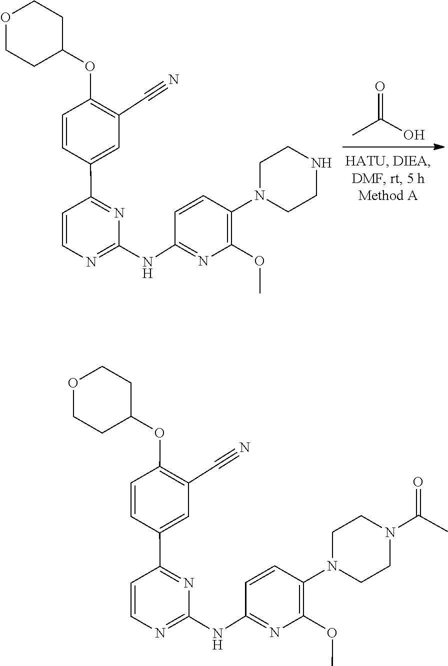

The title compound was prepared from 5-(2-(6-methoxy-5-(piperazin-1-yl)pyridin-2-ylamino)pyrimidin-4-yl)-2-(tetrahydro-2H-pyran-4-yloxy)benzonitrile and acetic acid using Method A. The final product was purified by prep-HPLC under the following conditions: column, XBridge Prep OBD C18 Column, 150×19 mm, 5 um; mobile phase, MeCN in water (with 10 mmol/L NH₄HCO₃ and 0.1% NH₃.H₂O), 25% to 55% gradient in 8 min; detector, UV 254 nm. 5-(2-[[5-(4-acetylpiperazin-1-yl)-6-methoxypyridin-2-yl]amino]pyrimidin-4-yl)-2-(oxan-4-yloxy)benzonitrile was obtained as an yellow solid (25 mg, 16%). HPLC: 98.7% purity, RT=5.18 min. MS: m/z=530.2 [M+H]⁺. ¹H NMR (300 MHz, DMSO-d6, ppm) δ 9.41 (s, 1H), 8.59-8.54 (m, 2H), 8.49-8.42 (m, 1H), 7.70-7.85 (m, 1H), 7.58-7.46 (m, 2H), 7.20-7.30 (m, 1H), 4.99-4.88 (m, 1H), 3.90 (s, 3H), 3.80-3.90 (m, 2H), 3.62-3.48 (m, 6H), 3.01-2.76 (m, 4H), 2.10-1.98 (m, 5H), 1.75-1.56 (m, 2H).

### Example 169: 5-{2-[6-Methoxy-5-(4-oxetan-3-yl-piperazin-1-yl)-pyridin-2-ylamino]-pyrimidin-4-yl}-2-(tetrahydro-pyran-4-yloxy)-benzonitrile

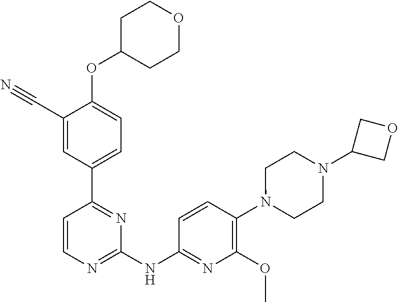 [SMILES: COc1nc(Nc2nccc(-c3ccc(OC4CCOCC4)c(C#N)c3)n2)ccc1N1CCN(C2COC2)CC1]

A mixture of 5-(2-aminopyrimidin-4-yl)-2-(oxan-4-yloxy)benzonitrile (175.00 mg; 0.59 mmol; 1.00 eq.), 1-(6-Bromo-2-methoxy-pyridin-3-yl)-4-oxetan-3-yl-piperazine (193.83 mg; 0.59 mmol; 1.00 eq.), Xantphos (129.03 mg; 0.20 mmol; 0.33 eq.), and Cs2CO3 (405.09 mg; 1.18 mmol; 2.00 eq.) in N,N-Dimethyl-formamide (15.00 ml; 220.68 mmol; 373.67 eq.) in a microwave vial was purged with argon for 15 min. Then Pd2(dba)₃CHCl3 (70.78 mg; 0.06 mmol; 0.11 eq.) was added. The reaction mixture was heated at 100° C. for 1 h under microwave irradiation. Filtered and DMF was removed. Methanol (20 ml) was added to the residue. The desired compound was precipitated which was recrystallized from methanol-DCM mixture to obtain 5-{2-[6-Methoxy-5-(4-oxetan-3-yl-piperazin-1-yl)-pyridin-2-ylamino]-pyrimidin-4-yl}-2-(tetrahydro-pyran-4-yloxy)-benzonitrile (150.00 mg; 46.7%) as a light yellow solid. HPLC: 100% purity, RT: 2.33; MS: m/z=544.1 [M+H]⁺. ¹H NMR (400 MHz, Chloroform-d) δ 8.53 (d, J=5.1 Hz, 1H), 8.35 (d, J=2.2 Hz, 1H), 8.27 (dd, J=9.0, 2.1 Hz, 1H), 7.89 (d, J=8.3 Hz, 1H), 7.68 (s, 1H), 7.31-7.22 (m, 1H), 7.17-7.06 (m, 2H), 4.87-4.61 (m, 5H), 4.06 (m, 2H), 3.99 (s, 3H), 3.73-3.58 (m, 3H), 3.13 (br s, 4H), 2.58 (br s, 4H), 2.11 (m, 2H), 1.96 (m, J=14.7, 7.4, 3.8 Hz, 2H).

### Example 170: 2-(3-Fluoro-tetrahydro-pyran-4-yloxy)-5-{2-[6-methoxy-5-(4-oxetan-3-yl-piperazin-1-yl)-pyridin-2-ylamino]-pyrimidin-4-yl}-benzonitrile

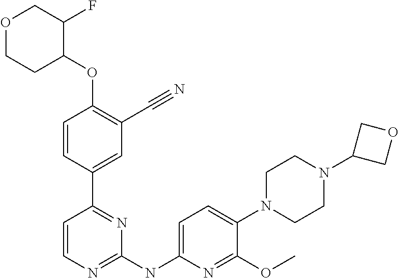 [SMILES: COc1nc(Nc2nccc(-c3ccc(OC4CCOCC4F)c(C#N)c3)n2)ccc1N1CCN(C2COC2)CC1]

A mixture of 5-(2-Amino-pyrimidin-4-yl)-2-(3-fluoro-tetrahydro-pyran-4-yloxy)-benzonitrile (200.00 mg; 0.64 mmol; 1.00 eq.), 1-(6-Bromo-2-methoxy-pyridin-3-yl)-4-oxetan-3-yl-piperazine (208.84 mg; 0.64 mmol; 1.00 eq.), 4,5-Bis-diphenylphosphanyl-9,9-dimethyl-9H-xanthene (0.12 ml; 0.19 mmol; 0.30 eq.), and Cesium carbonate (436.47 mg; 1.27 mmol; 2.00 eq.) in dioxane in a microwave vial was purged with argon for 3 min. Then Pd2(dba)₃CHCl3 (138.66 mg; 0.13 mmol; 0.20 eq.) was added. The reaction mixture was heated at 120° C. for overnight. After filtration, the solvent was removed and the mixture was purified by flash chromatography on silica gel (Hex: EtOAc from 100:0 to 0:100 and MeOH in EtOAc from 0% to 10%) to provide 2-(3-Fluoro-tetrahydro-pyran-4-yloxy)-5-{2-[6-methoxy-5-(4-oxetan-3-yl-piperazin-1-yl)-pyridin-2-ylamino]-pyrimidin-4-yl}-benzonitrile (83.60 mg in 23% yield), m/z: 562 (M+H). ¹H NMR (DMSO-d6): 9.36 (1H), 8.60 (1H), 8.52 (1H), 7.76 (1H), 7.62 (1H), 7.53 (1H), 7.29 (1H), 5.03 (1H), 4.89 (1H), 4.57 (2H), 4.48 (2H), 4.03 (1H), 3.90 (3H0, 3.79-3.56 (2H), 3.47 (1H), 2.98 (3H), 2.41 (3H), 1.99 (2H).

### Example 171: 2-(azetidin-3-yloxy)-5-[2-([4-[4-(oxetan-3-yl)piperazin-1-yl]phenyl]amino)pyrimidin-4-yl]benzonitrile and Example 172: 2-([1-[(2S)-2-hydroxypropanoyl]azetidin-3-yl]oxy)-5-[2-([4-[4-(oxetan-3-yl)piperazin-1-yl]phenyl]amino)pyrimidin-4-yl]benzonitrile

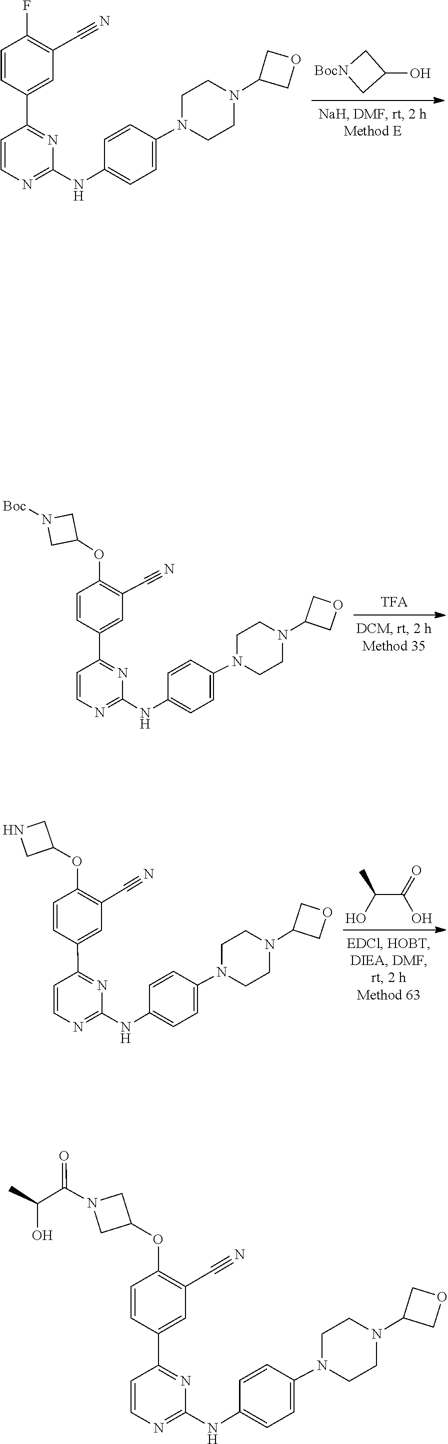

2-(azetidin-3-yloxy)-5-[2-([4-[4-(oxetan-3-yl)piperazin-1-yl]phenyl]amino)pyrimidin-4-yl]benzonitrile was prepared from tert-butyl 3-hydroxyazetidine-1-carboxylate and 2-fluoro-5-[2-([4-[4-(oxetan-3-yl)piperazin-1-yl]phenyl]amino)pyrimidin-4-yl]benzonitrile using Method E and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 16% to 46% gradient in 8 min; detector, UV 254 nm. 2-(azetidin-3-yloxy)-5-[2-([4-[4-(oxetan-3-yl)piperazin-1-yl]phenyl]amino)pyrimidin-4-yl]benzonitrile was obtained as a light yellow solid (4.8 mg, 7% for 2 steps). HPLC: 98.4% purity, RT=3.76 min. MS: m/z=556.2 [M+H]⁺. HPLC: 98.1% purity, RT=3.16 min. MS: m/z=484.1 [M+H]⁺. ¹H NMR (300 MHz, DMSO-d₆, ppm) δ 9.40 (s, 1H), 8.54-8.34 (m, 3H), 7.64-7.55 (m, 2H), 7.39-7.31 (m, 1H), 7.16-7.07 (m, 1H), 6.95-6.86 (m, 2H), 5.27-5.17 (m, 1H), 4.61-4.41 (m, 4H), 3.89-3.78 (m, 2H), 3.61-3.50 (m, 2H), 3.48-3.38 (m, 1H), 3.12-3.04 (m, 4H), 2.44-2.35 (m, 4H).

### Method 63

2-([1-[(2S)-2-hydroxypropanoyl]azetidin-3-yl]oxy)-5-[2-([4-[4-(oxetan-3-yl)piperazin-1-yl]phenyl]amino)pyrimidin-4-yl]benzonitrile: To a solution of 2-(azetidin-3-yloxy)-5-[2-([4-[4-(oxetan-3-yl)piperazin-1-yl]phenyl]amino)pyrimidin-4-yl]benzonitrile (93 mg, 0.19 mmol) in DMF (10 mL) was added (2S)-2-hydroxypropanoic acid (87 mg, 0.96 mmol), HOBT (52 mg, 0.38 mmol), EDC.HCl (73 mg, 0.38 mmol), and DIEA (248 mg, 1.92 mmol) at room temperature. The resulting mixture was stirred for 2 h at room temperature. When the reaction was done, the reaction was quenched by the addition of H₂O (100 mL). The resulting mixture was extracted with ethyl acetate (100 mL×3). The organic phases were combined, washed with brine and dried over Na₂SO₄. The solvent was concentrated under reduced pressure and the residue was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 17% to 47% gradient in 8 min; detector, UV 254 nm. 2-([1-[(2S)-2-hydroxypropanoyl]azetidin-3-yl]oxy)-5-[2-([4-[4-(oxetan-3-yl)piperazin-1-yl]phenyl]amino)pyrimidin-4-yl]benzonitrile was obtained as a yellow solid (35 mg, 32%). ¹H NMR (300 MHz, DMSO-do, ppm) δ 9.42 (s, 1H), 8.57-8.51 (m, 1H), 8.52-8.38 (m, 2H), 7.64-7.55 (m, 2H), 7.41-7.34 (m, 1H), 7.22-7.13 (m, 1H), 6.95-6.86 (m, 2H), 5.31-5.25 (m, 1H), 5.25-5.13 (m, 1H), 4.86-4.72 (m, 1H), 4.61-4.50 (m, 2H), 4.50-4.41 (m, 2H), 4.41-4.25 (m, 2H), 4.21-4.07 (m, 1H), 3.93-3.83 (m, 1H), 3.50-3.38 (m, 1H), 3.13-3.04 (m, 4H), 2.44-2.35 (m, 4H), 1.19 (d, J=6.7 Hz, 3H).

### Example 173: 2-([1-[(2S)-2-hydroxypropanoyl]azetidin-3-yl]oxy)-5-[2-([4-[4-(oxetan-3-yl)piperazin-1-yl]phenyl]amino)pyrimidin-4-yl]benzonitrile

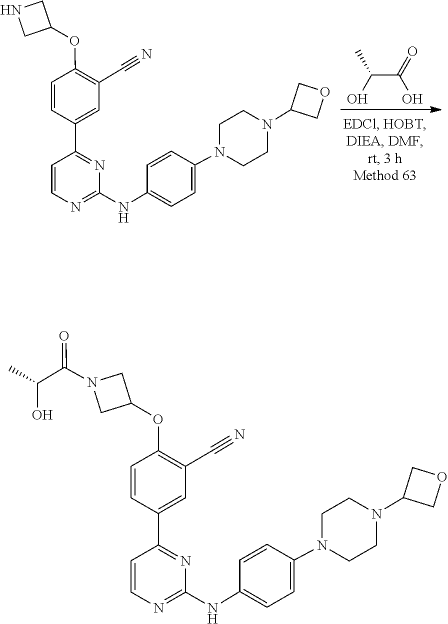

The title compound was prepared from (2R)-2-hydroxypropanoic acid and 2-(azetidin-3-yloxy)-5-[2-([4-[4-(oxetan-3-yl)piperazin-1-yl]phenyl]amino)pyrimidin-4-yl]benzonitrile using Method 63. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 17% to 47% gradient in 8 min; detector, UV 254 nm. 2-([1-[(2R)-2-hydroxypropanoyl]azetidin-3-yl]oxy)-5-[2-([4-[4-(oxetan-3-yl)piperazin-1-yl]phenyl]amino)pyrimidin-4-yl]benzonitrile was obtained as an yellow solid (28 mg, 37%). HPLC: 98.1% purity, RT=3.64 min. MS: m/z=556.1 [M+H]⁺. ¹H NMR (300 MHz, DMSO-d₆, ppm) δ9.42 (s, 1H), 8.57-8.51 (m, 1H), 8.51-8.37 (m, 2H), 7.64-7.55 (m, 2H), 7.41-7.34 (m, 1H), 7.22-7.13 (m, 1H), 6.95-6.86 (m, 2H), 5.31-5.25 (m, 1H), 5.25-5.13 (m, 1H), 4.86-4.71 (m, 1H), 4.61-4.50 (m, 2H), 4.50-4.41 (m, 2H), 4.42-4.24 (m, 2H), 4.21-4.07 (m, 1H), 3.88-3.78 (m, 1H), 3.50-3.36 (m, 1H), 3.13-3.04 (m, 4H), 2.44-2.35 (m, 4H), 1.19 (d, J=6.7 Hz, 3H).

### Example 174: 2-(azetidin-3-yloxy)-5-[2-([6-methoxy-5-[4-(oxetan-3-yl)piperazin-1-yl]pyridin-2-yl]amino)pyrimidin-4-yl]benzonitrile (MSC2695698) and Example 175: 2-([1-[(2S)-2-hydroxypropanoyl]azetidin-3-yl]oxy)-5-[2-([6-methoxy-5-[4-(oxetan-3-yl)piperazin-1-yl]pyridin-2-yl]amino)pyrimidin-4-yl]benzonitrile

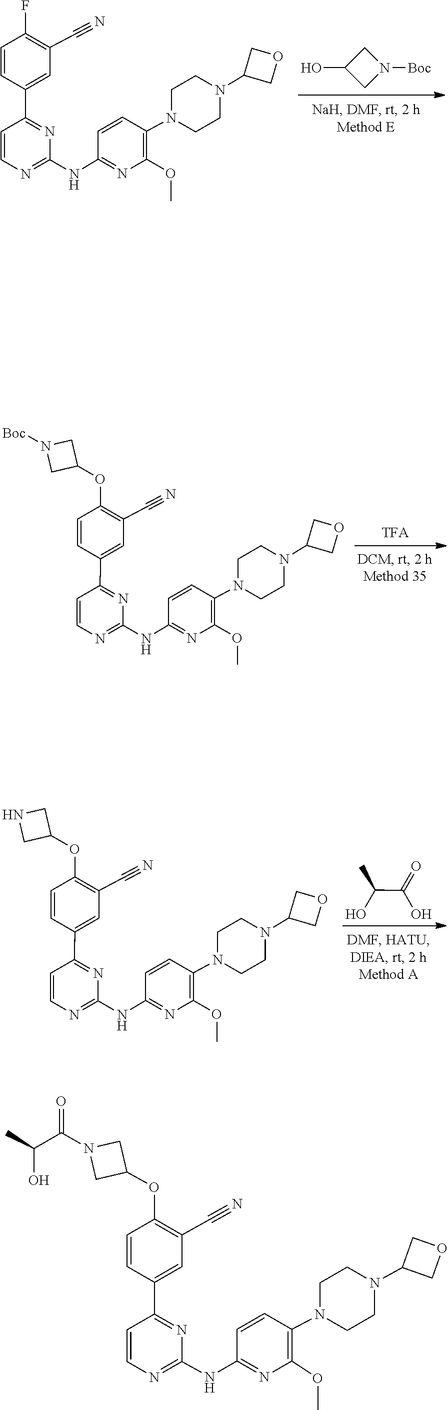

2-(azetidin-3-yloxy)-5-[2-([6-methoxy-5-[4-(oxetan-3-yl)piperazin-1-yl]pyridin-2-yl]amino)pyrimidin-4-yl]benzonitrile was prepared from tert-butyl 3-hydroxyazetidine-1-carboxylate and 2-fluoro-5-[2-([6-methoxy-5-[4-(oxetan-3-yl)piperazin-1-yl]pyridin-2-yl]amino)pyrimidin-4-yl]benzonitrile using Method E and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 30% to 60% gradient in 8 min; detector, UV 254 nm. 2-(azetidin-3-yloxy)-5-[2-([6-methoxy-5-[4-(oxetan-3-yl)piperazin-1-yl]pyridin-2-yl]amino)pyrimidin-4-yl]benzonitrile was obtained as a light yellow solid (3 mg, 6.6% for 2 steps). HPLC: 96.0% purity, RT=3.15 min. MS: m/z=515.0 [M+H]⁺. ¹H NMR (300 MHz, DMSO-d₆.ppm) δ9.35 (s, 1H), 8.60-8.52 (m, 2H), 8.48-8.39 (m, 1H), 7.77-7.68 (m, 1H), 7.53-7.45 (m, 1H), 7.31-7.22 (m, 1H), 7.17-7.08 (m, 1H), 5.28-5.18 (m, 1H), 4.54 (t, J=6.4 Hz, 2H), 4.45 (t, J=6.1 Hz, 2H), 3.91-3.78 (m, 5H), 3.61-3.51 (m, 2H), 3.50-3.40 (m, 1H), 2.99-2.93 (m, 4H), 2.80-2.74 (m, 1H), 2.42-2.36 (m, 4H).

2-([1-[(2S)-2-hydroxypropanoyl]azetidin-3-yl]oxy)-5-[2-([6-methoxy-5-[4-(oxetan-3-yl)piperazin-1-yl]pyridin-2-yl]amino)pyrimidin-4-yl]benzonitrile: The title compound was prepared from 2-(azetidin-3-yloxy)-5-[2-([6-methoxy-5-[4-(oxetan-3-yl)piperazin-1-yl]pyridin-2-yl]amino)pyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 17% to 47% gradient in 8 min; detector, UV 254 nm. 2-([1-[(2S)-2-hydroxypropanoyl]azetidin-3-yl]oxy)-5-[2-([6-methoxy-5-[4-(oxetan-3-yl)piperazin-1-yl]pyridin-2-yl]amino)pyrimidin-4-yl]benzonitrile was obtained as an yellow solid (30 mg, 36%). HPLC: 96.0% purity, RT=3.69 min. MS: m/z=578.3 [M+H]⁺. ¹H NMR (300 MHz, DMSO-d₆.ppm) δ 9.37 (s, 1H), 8.64-8.53 (m, 2H), 8.52-8.42 (m, 1H), 7.77-7.68 (m, 1H), 7.55-7.47 (m, 1H), 7.33-7.14 (m, 2H), 5.33-5.27 (m, 1H), 5.26-5.14 (m, 1H), 4.87-4.72 (m, 1H), 4.65-4.25 (m, 6H), 4.22-4.09 (m, 1H), 3.88 (s, 3H), 3.91-3.82 (m, 1H), 3.50-3.40 (m, 1H), 3.00-2.94 (m, 4H), 2.42-2.36 (m, 4H), 1.19 (d, J=6.7 Hz, 3H).

### Example 176: 2-([1-[(2R)-2-hydroxypropanoyl]azetidin-3-yl]oxy)-5-[2-([6-methoxy-5-[4-(oxetan-3-yl)piperazin-1-yl]pyridin-2-yl]amino)pyrimidin-4-yl]benzonitrile

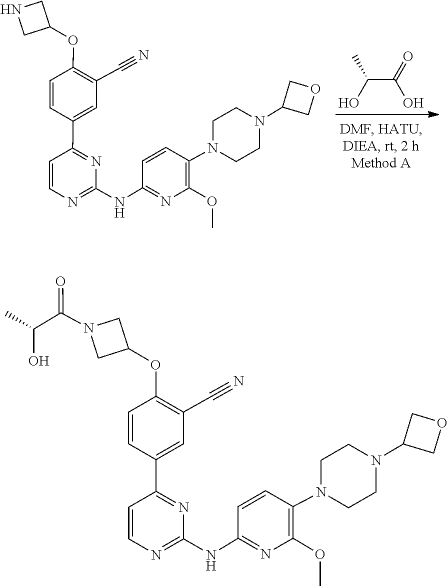

The title compound was prepared from 2-(azetidin-3-yloxy)-5-[2-([6-methoxy-5-[4-(oxetan-3-yl)piperazin-1-yl]pyridin-2-yl]amino)pyrimidin-4-yl]benzonitrile and (2R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 17% to 47% gradient in 8 min; detector, UV 254 nm. 2-([1-[(2R)-2-hydroxypropanoyl]azetidin-3-yl]oxy)-5-[2-([6-methoxy-5-[4-(oxetan-3-yl)piperazin-1-yl]pyridin-2-yl]amino)pyrimidin-4-yl]benzonitrile was obtained as an yellow solid (27 mg, 37%). HPLC: 99.0% purity, RT=3.71 min. MS: m/z=587.1 [M+H]⁺. ¹H NMR (300 MHz, DMSO-d₆.ppm) δ 9.37 (s, 1H), 8.64-8.53 (m, 2H), 8.52-8.42 (m, 1H), 7.77-7.68 (m, 1H), 7.55-7.47 (m, 1H), 7.31-7.22 (m, 1H), 7.22-7.14 (m, 1H), 5.37-5.14 (m, 2H), 4.81-4.75 (m, 1H), 4.62-4.26 (m, 6H), 4.21-4.08 (m, 1H), 3.97-3.84 (m, 4H), 3.50-3.40 (m, 1H), 3.00-2.94 (m, 4H), 2.42-2.36 (m, 4H), 1.19 (d, J=6.7 Hz, 3H).

### Example 177: 5-(2-(4-(4-(oxetan-3-yl)piperazin-1-yl)phenylamino)pyrimidin-4-yl)-2-(pyrrolidin-3-yloxy)benzonitrile and Example 178: 2-([1-[(2S)-2-hydroxypropanoyl]pyrrolidin-3-yl]oxy)-5-[2-([4-[4-(oxetan-3-yl)piperazin-1-yl]phenyl]amino)pyrimidin-4-yl]benzonitrile

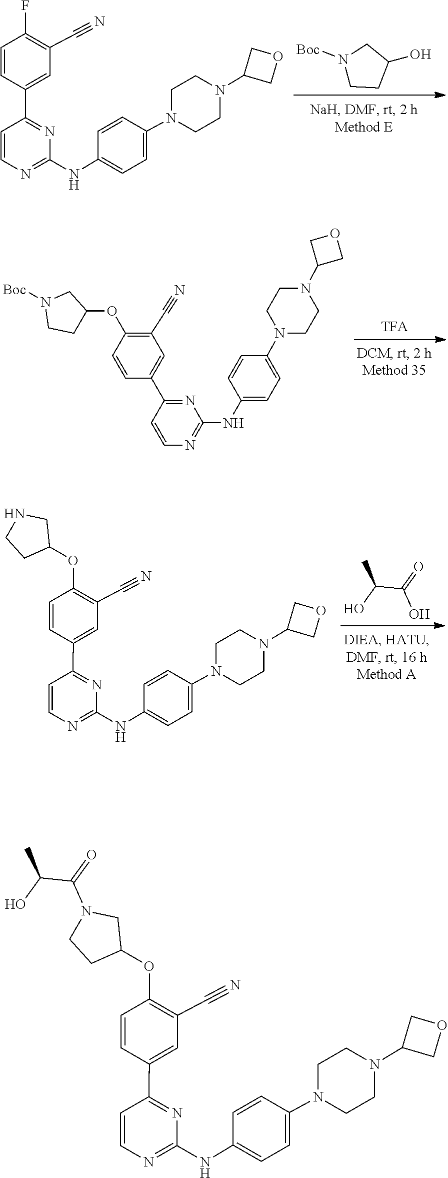

5-(2-(4-(4-(oxetan-3-yl)piperazin-1-yl)phenylamino)pyrimidin-4-yl)-2-(pyrrolidin-3-yloxy)benzonitrile was prepared from 2-fluoro-5-(2-(4-(4-(oxetan-3-yl)piperazin-1-yl)phenylamino)pyrimidin-4-yl)benzonitrile and tert-butyl 3-hydroxypyrrolidine-1-carboxylate using Method E and 35. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 22% to 36% gradient in 8 min; detector, UV 254 nm. 5-(2-(4-(4-(oxetan-3-yl)piperazin-1-yl)phenylamino)pyrimidin-4-yl)-2-(pyrrolidin-3-yloxy)benzonitrile was obtained as an yellow solid (16 mg, 24% for 2 steps). HPLC: 95.9% purity, RT=3.36 min. MS: m/z=498.2[M+H]⁺. ¹H NMR (300 MHz, DMSO-d₆, ppm) δ 9.40 (s, 1H), 8.52-8.36 (m, 3H), 7.65-7.55 (m, 2H), 7.44-7.32 (m, 2H), 6.95-6.86 (m, 2H), 5.15 (brs, 1H), 4.61-4.50 (m, 2H), 4.51-4.40 (m, 2H), 3.56-3.15 (m, 2H), 3.14-3.04 (m, 3H), 3.02-2.79 (m, 1H), 2.45-2.35 (m, 4H), 2.19-2.03 (m, 1H), 1.93-1.82 (m, 1H).

2-([1-[(2S)-2-hydroxypropanoyl]pyrrolidin-3-yl]oxy)-5-[2-([4-[4-(oxetan-3-yl)piperazin-1-yl]phenyl]amino)pyrimidin-4-yl]benzonitrile: The title compound was prepared from 5-[2-([4-[4-(oxetan-3-yl)piperazin-1-yl]phenyl]amino)pyrimidin-4-yl]-2-(pyrrolidin-3-yloxy)benzonitrile and (S)-2-hydroxypropanoic acid using Method A. The final product was purified by prep-HPLC under the following condition: Column, XBridge Prep OBD C18 Column, 150×30 mm, 5 um; mobile phase, acetonitrile in water (with 10 mmol/L NR₄HCO₃ and 0.1% NH₃.H₂O), 23% to 37% gradient in 8 min; detector, UV 254 nm. 2-([1-[(2S)-2-hydroxypropanoyl]pyrrolidin-3-yl]oxy)-5-[2-([4-[4-(oxetan-3-yl)piperazin-1-yl]phenyl]amino)pyrimidin-4-yl]benzonitrile was obtained as an yellow solid (19 mg, 17%). HPLC: 99.0% purity, RT=3.68 min. MS: m/z=549.1[M+H]⁺. ¹H NMR (300 MHz, DMSO-d₆, ppm) δ 9.41 (s, 1H), 8.53-8.40 (m, 3H), 7.65-7.55 (m, 2H), 7.54-7.44 (m, 1H), 7.42-7.33 (m, 1H), 6.95-6.86 (m, 2H), 5.41-5.28 (m, 1H), 5.00-4.93 (m, 1H), 4.61-4.50 (m, 2H), 4.51-4.40 (m, 2H), 4.36-4.19 (m, 1H), 3.96-3.85 (m, 1H), 3.74-3.56 (m, 2H), 3.49-3.38 (m, 1H), 3.12-3.05 (m, 4H), 2.43-2.36 (m, 4H), 2.30-2.05 (m, 2H), 1.23-1.10 (m, 3H).

### Example 179: 2-([1-[(2R)-2-hydroxypropanoyl]pyrrolidin-3-yl]oxy)-5-[2-([4-[4-(oxetan-3-yl)piperazin-1-yl]phenyl]amino)pyrimidin-4-yl]benzonitrile

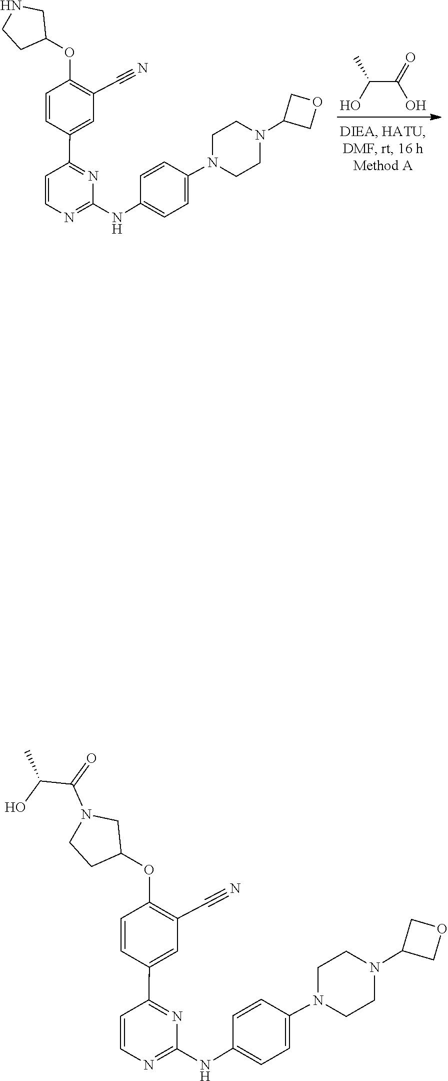

The title compound was prepared from 5-[2-([4-[4-(oxetan-3-yl)piperazin-1-yl]phenyl]amino)pyrimidin-4-yl]-2-(pyrrolidin-3-yloxy)benzonitrile and (R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 20% to 45% gradient in 8 min; detector, UV 254 nm. 2-([1-[(2R)-2-hydroxypropanoyl]pyrrolidin-3-yl]oxy)-5-[2-([4-[4-(oxetan-3-yl)piperazin-1-yl]phenyl]amino)pyrimidin-4-yl]benzonitrile was obtained as an yellow solid (19 mg, 17%). HPLC: 98.6% purity, RT=3.83 min. MS: m/z=570.1 [M+H]⁺. ¹H NMR (300 MHz, DMSO-d₆, ppm) δ 9.40 (s, 1H), 8.54-8.39 (m, 3H), 7.65-7.55 (m, 2H), 7.54-7.44 (m, 1H), 7.41-7.33 (m, 1H), 6.95-6.86 (m, 2H), 5.41-5.34 (m, 1H), 5.01-4.86 (m, 1H), 4.61-4.50 (m, 2H), 4.51-4.41 (m, 2H), 4.35-4.19 (m, 1H), 4.01-3.36 (m, 5H), 3.14-3.04 (m, 4H), 2.45-2.35 (m, 4H), 2.34-2.03 (m, 2H), 1.24-1.10 (m, 3H).

### Example 180: 5-[2-([4-[4-(oxetan-3-yl)piperazin-1-yl]phenyl]amino)pyrimidin-4-yl]-2-(oxolan-3-yloxy)benzonitrile

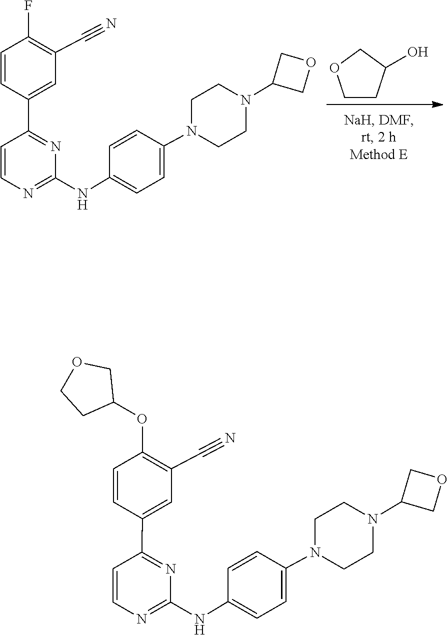

The title compound was prepared from 2-fluoro-5-[2-([4-[4-(oxetan-3-yl)piperazin-1-yl]phenyl]amino)pyrimidin-4-yl]benzonitrile and oxolan-3-ol using Method E. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 30% to 60% gradient in 8 min; detector, UV 254 nm. 5-[2-([4-[4-(oxetan-3-yl)piperazin-1-yl]phenyl]amino)pyrimidin-4-yl]-2-(oxolan-3-yloxy)benzonitrile was obtained as a light yellow solid (26 mg, 19%). HPLC: 98.8% purity, RT=4.34 min. MS: m/z=499.1 [M+H]⁺. ¹H NMR (400 MHz, DMSO-d₆, ppm) δ 9.41 (s, 1H), 8.54-8.40 (m, 3H), 7.65-7.58 (m, 2H), 7.46-7.35 (m, 2H), 6.98-6.89 (m, 2H), 5.36-5.30 (m, 1H), 4.62-4.53 (m, 2 H), 4.52-4.44 (m, 2H), 4.00-3.85 (m, 3H), 3.85-3.75 (m, 1H), 3.48-3.43 (m, 1H), 3.13-3.08 (m, 4H), 2.44-2.39 (m, 4H), 2.39-2.27 (m, 1H), 2.11-2.01 (m, 1H).

### Example 181: 2-([1-[(2R)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile

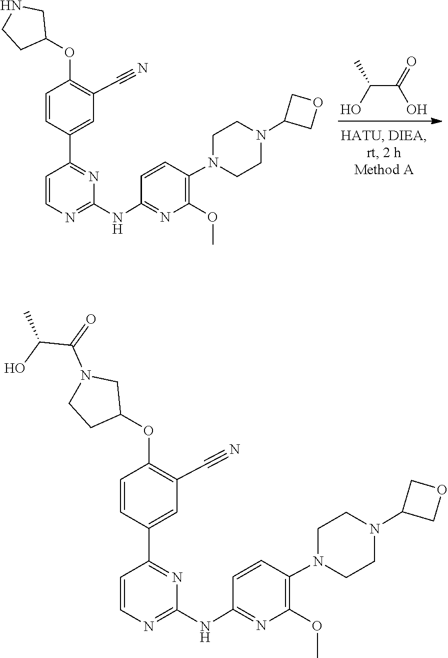

The title compound was prepared from 5-(2-(6-methoxy-5-(4-(oxetan-3-yl)piperazin-1-yl)pyridin-2-ylamino)pyrimidin-4-yl)-2-(pyrrolidin-3-yloxy)benzonitrile and (R)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 25% to 45% gradient in 8 min; detector, UV 254 nm. 2-([1-[(2R)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile was obtained as an yellow solid (25 mg, 24%). HPLC: 99.7% purity, RT=3.77 min. MS: m/z=601.2 [M+H]⁺. ¹H NMR (300 MHz, DMSO-d₆.ppm) δ 9.35 (s, 1H), 8.60-8.54 (m, 2H), 8.54-8.45 (m, 1H), 7.73 (d, J=8.3 Hz, 1H), 7.55-7.47 (m, 2H), 7.27 (d, J=8.3 Hz, 1H), 5.42-5.36 (m, 1H), 5.01-4.87 (m, 1H), 4.60-4.50 (m, 2H), 4.50-4.40 (m, 2H), 4.37-4.18 (m, 1H), 3.90-3.85 (m, 4H), 3.80-3.35 (m, 4H), 3.00-2.94 (m, 4H), 2.42-2.36 (m, 4H), 2.30-2.07 (m, 2H), 1.24-1.11 (m, 3H).

### Example 182: 5-[2-([6-methoxy-5-[4-(oxetan-3-yl)piperazin-1-yl]pyridin-2-yl]amino)pyrimidin-4-yl]-2-(pyrrolidin-3-yloxy)benzonitrile and Example 183: 2-([1-[(2S)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile

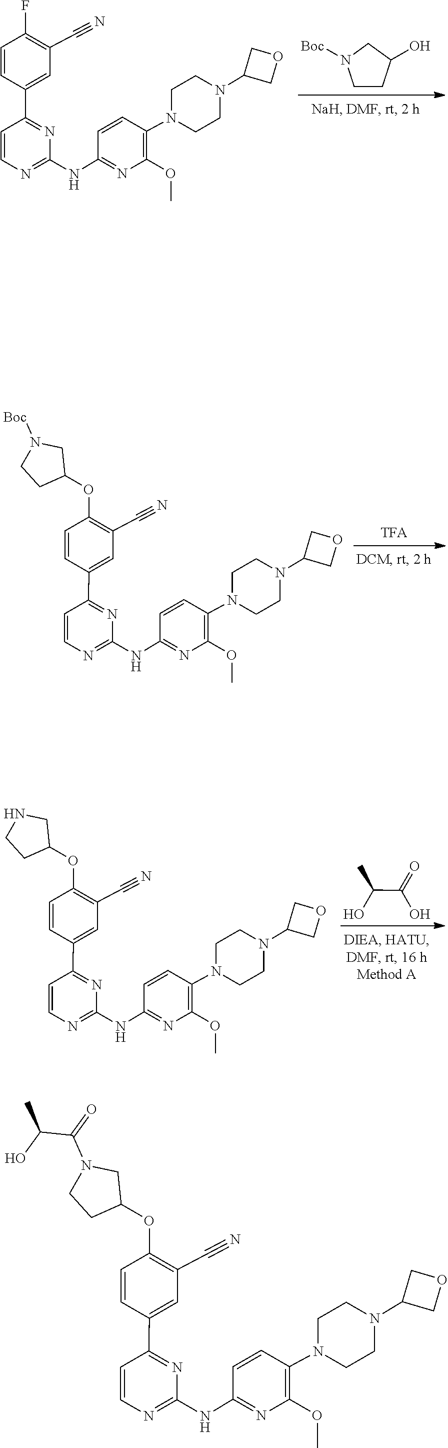

5-[2-([6-methoxy-5-[4-(oxetan-3-yl)piperazin-1-yl]pyridin-2-yl]amino)pyrimidin-4-yl]-2-(pyrrolidin-3-yloxy)benzonitrile was prepared from 2-fluoro-5-(2-(6-methoxy-5-(4-(oxetan-3-yl)piperazin-1-yl)pyridin-2-ylamino)pyrimidin-4-yl)benzonitrile and tert-butyl 3-hydroxypyrrolidine-1-carboxylate using Method E and 35. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 20% to 45% gradient in 8 min; detector, UV 254 nm. 5-[2-([6-methoxy-5-[4-(oxetan-3-yl)piperazin-1-yl]pyridin-2-yl]amino)pyrimidin-4-yl]-2-(pyrrolidin-3-yloxy)benzonitrile was obtained as an yellow solid (6 mg, 15% for 2 steps). HPLC: 96.8% purity, RT=3.20 min. MS: m/z=529.2 [M+H]⁺. ¹H NMR (300 MHz, DMSO-d6, ppm) δ 9.34 (s, 1H), 8.59-8.51 (m, 2H), 8.51-8.41 (m, 1H), 7.77-7.69 (m, 1H), 7.53-7.45 (m, 1H), 7.44-7.35 (m, 1H), 7.31-7.22 (m, 1H), 5.12 (br s, 1H), 4.59-4.49 (m, 2H), 4.49-4.40 (m, 2H), 3.88 (s, 3H), 3.52-3.40 (m, 1H), 3.22-3.10 (m, 1H), 3.07-2.70 (m, 7H), 2.42-2.36 (m, 4H), 2.19-2.05 (m, 1H), 1.89-1.75 (m, 1H).

2-([1-[(2S)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile: The title compound was prepared from 2-fluoro-5-(2-(6-methoxy-5-(4-(oxetan-3-yl)piperazin-1-yl)pyridin-2-ylamino)pyrimidin-4-yl)benzonitrile, tert-butyl 3-hydroxypyrrolidine-1-carboxylate and (S)-2-hydroxypropanoic acid using Method E, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 25% to 45% gradient in 8 min; detector, UV 254 nm. 2-([1-[(2S)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile was obtained as an yellow solid (27 mg, 11% for 3 steps). HPLC: 99.7% purity, RT=3.77 min. MS: m/z=601.2 [M+H]1H NMR (300 MHz, DMSO-d6, ppm) δ9.36 (s, 1H), 8.60-8.53 (m, 2H), 8.53-8.45 (m, 1H), 7.73 (d, J=8.3 Hz, 1H), 7.51 (d, J=5.4 Hz, 2H), 7.27 (d, J=8.3 Hz, 1H), 5.42-5.36 (m, 1H), 5.02-4.87 (m, 1H), 4.60-4.50 (m, 2H), 4.50-4.40 (m, 2H), 4.37-4.17 (m, 1H), 3.90-3.86 (m, 4H), 3.84-3.38 (m, 4H), 3.00-2.94 (m, 4H), 2.42-2.36 (m, 4H), 2.18-2.12 (m, 2H), 1.23-1.10 (m, 3H).

### Example 184: 2-([1-[(2R)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile

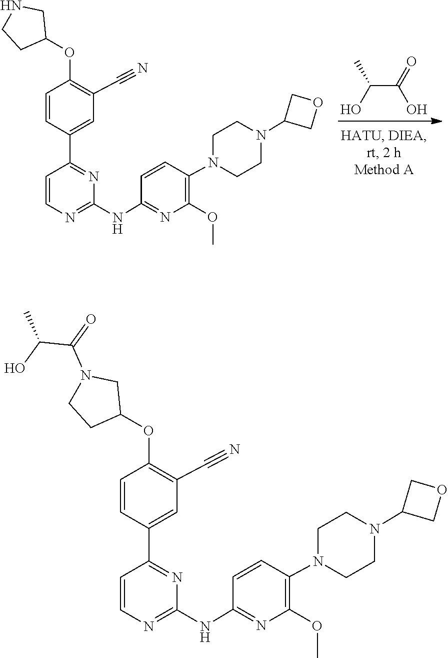

The title compound was prepared from 5-(2-(6-methoxy-5-(4-(oxetan-3-yl)piperazin-1-yl)pyridin-2-ylamino)pyrimidin-4-yl)-2-(pyrrolidin-3-yloxy)benzonitrile and (R)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₁HCO₃ and 0.1% NH₃.H₂O), 25% to 45% gradient in 8 min; detector, UV 254 nm. 2-([1-[(2R)-2-hydroxypropanoyl]pyrrolidin-3-yl]oxy)-5-[2-([6-methoxy-5-[4-(oxetan-3-yl)piperazin-1-yl]pyridin-2-yl]amino)pyrimidin-4-yl]benzonitrile was obtained as an yellow solid (25 mg, 24%). HPLC: 99.7% purity, RT=3.77 min. MS: m/z=601.2 [M+H]⁺. ¹H NMR (300 MHz, DMSO-d₆, ppm) δ 9.35 (s, 1H), 8.60-8.54 (m, 2H), 8.54-8.45 (m, 1H), 7.73 (d, J=8.3 Hz, 1H), 7.55-7.47 (m, 2H), 7.27 (d, J=8.3 Hz, 1H), 5.42-5.36 (m, 1H), 5.01-4.87 (m, 1H), 4.60-4.50 (m, 2H), 4.50-4.40 (m, 2H), 4.37-4.18 (m, 1H), 3.90-3.85 (m, 4H), 3.80-3.35 (m, 4H), 3.00-2.94 (m, 4H), 2.42-2.36 (m, 4H), 2.30-2.07 (m, 2H), 1.24-1.11 (m, 3H).

### Example 185: 5-[2-({6-methoxy-5-[4-(oxetan-3-yl)piperazin-1-yl]pyridin-2-yl}amino)pyrimidin-4-yl]-2-(piperidin-4-yloxy)benzonitrile and Example 186: 2-(1-(2-hydroxyacetyl)piperidin-4-yloxy)-5-(2-(6-methoxy-5-(4-(oxetan-3-yl)piperazin-1-yl)pyridin-2-ylamino)pyrimidin-4-yl)benzonitrile

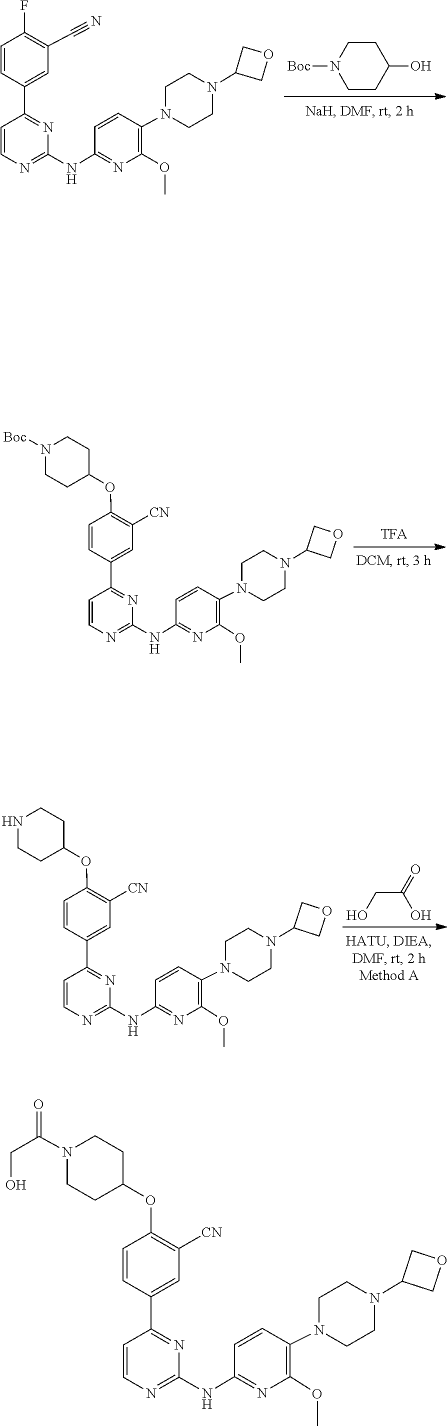

The title compounds were prepared from 2-fluoro-5-(2-(6-methoxy-5-(4-(oxetan-3-yl)piperazin-1-yl)pyridin-2-ylamino)pyrimidin-4-yl)benzonitrile, tert-butyl 4-hydroxypiperidine-1-carboxylate and 2-hydroxy acetic acid using Method E, 35 A. The final product was purified by prep-HPLC under the following conditions: column, XBridge Prep OBD C18 Column, 150×30 mm, 5 um; mobile phase, acetonitrile in water (with 10 mmol/L NR₄HCO₃), 25% to 55% gradient in 8 min; detector, UV 254 nm. 2-(1-(2-hydroxyacetyl)piperidin-4-yloxy)-5-(2-(6-methoxy-5-(4-(oxetan-3-yl)piperazin-1-yl)pyridin-2-ylamino)pyrimidin-4-yl)benzonitrile was obtained as an yellow solid (24 mg, 15% for 3 steps). HPLC: 99.2% purity, RT=3.90 min. MS: m/z=601.2 [M+H]⁺. ¹H NMR (300 MHz, DMSO-d₆, ppm) δ 9.36 (s, 1H), 8.60-8.52 (m, 2H), 8.47 (dd, J=9.0, 2.3 Hz, 1H), 7.73 (d, J=8.3 Hz, 1H), 7.59-7.47 (m, 2H), 7.26 (d, J=8.4 Hz, 1H), 5.12-4.94 (m, 1H), 4.60-4.39 (m, 5H), 4.12 (d, J=5.5 Hz, 2H), 3.88 (s, 3H), 3.78-3.34 (m, 5H), 3.00-2.94 (m, 4H), 2.42-2.36 (m, 4H), 2.01-1.90 (m, 2H), 1.73-1.67 (m, 2H).

### Example 187: 2-([1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile

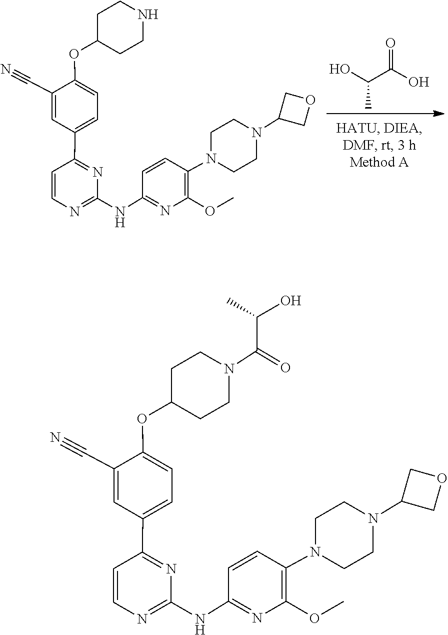

The title compound was prepared from 5-bromo-2-chloropyridine, 1-(oxetan-3-yl)piperazine, tert-butyl 4-(4-(2-aminopyrimidin-4-yl)-2-cyanophenoxy)-3,3-difluoropiperidine-1-carboxylate and (S)-2-hydroxypropanoic acid using Method A. The final product was purified by prep-HPLC under the following conditions: column, XBridge Prep OBD C18 Column, 150×19 mm, 5 um; mobile phase, EtOH in water (with 10 mmol/L NR₄HCO₃ and 0.1% NH₃.H₂O), 33% to 55% gradient in 8 min; detector, UV 254 nm. 2-([1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile was obtained as an yellow solid (32 mg, 31%). HPLC: 96.2% purity, RT=4.00 min. MS: m/z=615.3 [M+H]⁺. ¹H NMR (300 MHz, DMSO-d6, ppm) δ 9.35 (s, 1H), 8.60-8.52 (m, 2H), 8.52-8.42 (m, 1H), 7.75-7.71 (m, 1H), 7.59-7.46 (m, 2H), 7.26 (d, J=8.4 Hz, 1H), 5.06-4.84 (m, 2H), 4.62-4.50 (m, 2H), 4.49-4.39 (m, 3H), 3.94 (s, 3H), 3.83-3.63 (m, 2H), 3.60-3.39 (m, 3H), 3.02-2.94 (m, 4H), 2.41-2.32 (m, 4H), 2.02-1.96 (m, 2H), 1.74-1.68 (m, 2H), 1.19 (d, J=6.5 Hz, 3H).

### Example 188: 2-[(3S,4S)-3-Fluoro-1-((R)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

### Intermediate: 2-((3S,4S)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

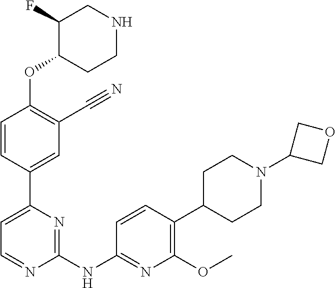 [SMILES: COc1nc(Nc2nccc(-c3ccc(O[C@H]4CCNC[C@@H]4F)c(C#N)c3)n2)ccc1C1CCN(C2COC2)CC1]

The title compound (460 mg, 100%) was synthesized using (3S,4S)-4-{2-Cyano-4-[2-(2-methoxy-1′-oxetan-3-yl-1′,2′,3′,4′,5′,6′-hexahydro-[3,4′]bipyridinyl-6-ylamino)-pyrimidin-4-yl]-phenoxy}-3-fluoro-piperidine-1-carboxylic acid tert-butyl ester (500.00 mg; 0.76 mmol; 1.00 eq) and TFA (2.90 mL, 37.89 mmol) in DCM. m/z: 560 (M+H). ¹H NMR (400 MHz, DMSO-d6) δ 10.69 (s, 1H), 9.61 (s, 1H), 9.18 (d, J=49.1 Hz, 3H), 8.64 (d, J=3.7 Hz, 3H), 8.54 (d, J=8.7 Hz, 1H), 7.84 (d, J=8.0 Hz, 1H), 7.62 (dd, J=14.6, 7.2 Hz, 3H), 7.53 (d, J=8.1 Hz, 1H), 7.32-6.96 (m, 1H), 6.08 (s, OH), 5.20 (s, 1H), 5.02 (d, J=5.0 Hz, 1H), 4.78 (dt, J=18.8, 7.6 Hz, 5H), 4.39 (s, 1H), 3.95-3.89 (m, 4H), 3.21 (s, 3H), 3.05-2.94 (m, 4H), 2.33 (d, J=27.9 Hz, 0H), 2.07-1.81 (m, 7H).

### 2-[(3S,4S)-3-Fluoro-1-((R)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

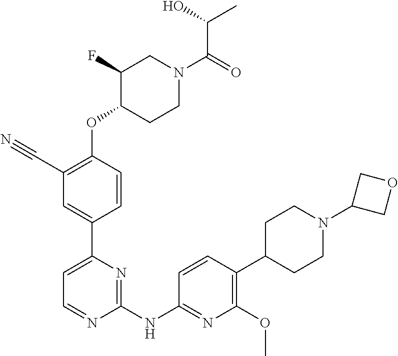 [SMILES: COc1nc(Nc2nccc(-c3ccc(O[C@H]4CCN(C(=O)[C@@H](C)O)C[C@@H]4F)c(C#N)c3)n2)ccc1C1CCN(C2COC2)CC1]

The title compound (5 mg, 2%) was synthesized using 2-((3S,4S)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (200.00 mg; 0.36 mmol; 1.00 eq.), (R)-2-Hydroxy-propionic acid (64.38 mg; 0.71 mmol; 2.00 eq.), 0-(7-Azabenzotriazol-1-yl)-N,N,N′,N′-tetramethyluronium hexaflurophosphate (HATU) (238.43 mg; 0.63 mmol; 1.73 eq.) and Ethyl-diisopropyl-amine (230.94 mg; 1.79 mmol; 5.00 eq.). m/z: 632 (M+H). ¹H NMR (400 MHz, DMSO-d6) δ 9.50 (s, 1H), 8.62 (d, J=5.8 Hz, 2H), 8.52 (d, J=8.9 Hz, 1H), 7.82 (d, J=7.9 Hz, 1H), 7.61 (dd, J=25.8, 7.3 Hz, 3H), 5.11 (d, J=18.3 Hz, 2H), 4.76 (s, 2H), 4.57 (s, 2H), 4.48 (s, 3H), 4.10 (dt, J=33.4, 16.0 Hz, 1H), 3.91 (s, 3H), 3.61 (dt, J=14.1, 7.5 Hz, 1H), 3.52 (s, 1H), 3.42 (s, 1H), 2.80 (s, 2H), 2.54 (d, J=1.7 Hz, 1H), 2.16 (s, 1H), 1.73 (s, 6H), 1.22 (d, J=6.3 Hz, 4H).

### Example 189: 2-[(3S,4S)-3-Fluoro-1-((S)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

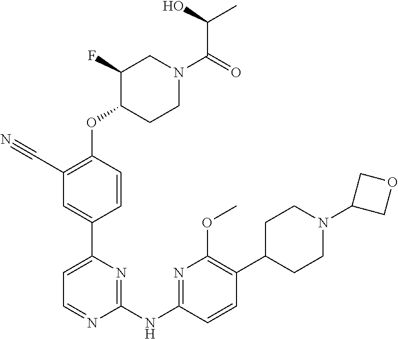 [SMILES: COc1nc(Nc2nccc(-c3ccc(O[C@H]4CCN(C(=O)[C@H](C)O)C[C@@H]4F)c(C#N)c3)n2)ccc1C1CCN(C2COC2)CC1]

The title compound (25 mg) was synthesized using 2-((3S,4S)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (200.00 mg; 0.36 mmol; 1.00 eq (S)-2-Hydroxy-propionic acid (64.38 mg; 0.71 mmol; 2.00 eq.), 0-(7-Azabenzotriazol-1-yl)-N,N,N′,N′-tetramethyluronium hexaflurophosphate (HATU) (238.43 mg; 0.63 mmol; 1.75 eq.) and Ethyl-diisopropyl-amine (0.18 ml; 1.79 mmol; 5.00 eq.) in 9% yield, m/z: 632 (M+H). ¹H NMR (400 MHz, DMSO-d6) δ 9.51 (s, 1H), 8.62 (d, J=5.7 Hz, 2H), 8.52 (dd, J=8.9, 2.4 Hz, 1H), 7.82 (d, J=8.1 Hz, 1H), 7.68-7.54 (m, 3H), 5.13 (s, 1H), 4.77 (s, 1H), 4.64 (s, 2H), 4.56 (s, 2H), 4.53-4.44 (m, 1H), 4.15 (dd, J=30.1, 15.2 Hz, 1H), 3.91 (s, 2H), 3.50 (s, 2H), 2.80 (s, 2H), 2.54 (d, J=1.4 Hz, 1H), 2.16 (s, 1H), 1.73 (s, 6H) 1.23 (s, 2H), 1.23 (d, J=13.5 Hz, 1H).

### Example 190: 2-[[(3R,4S)-3-fluoro-1-(2-hydroxypropanoyl)piperidin-4-yl]oxy]-5-[2-([6-methoxy-5-[4-(oxetan-3-yl)piperazin-1-yl]pyridin-2-yl]amino)pyrimidin-4-yl]benzonitrile

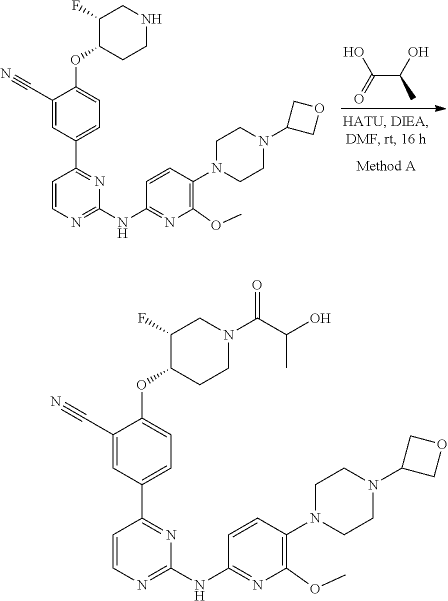

The title compound was prepared from 2-[[(3R,4S)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile and 2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 20% to 50% gradient in 8 min; detector, UV 254 nm. 2-[[(3R,4S)-3-fluoro-1-(2-hydroxypropanoyl)piperidin-4-yl]oxy]-5-[2-([6-methoxy-5-[4-(oxetan-3-yl)piperazin-1-yl]pyridin-2-yl]amino)pyrimidin-4-yl]benzonitrile was obtained as a light yellow solid (27 mg, 18%). HPLC: 98.3% purity, RT=2.97 min. MS: m/z=633.2 [M+H]⁺. ¹H NMR (300 MHz, DMSO-d₆, ppm) δ 9.35 (s, 1H), 8.65-8.53 (m, 2H), 8.53-8.44 (m, 1H), 7.78-7.68 (m, 1H), 7.67-7.58 (m, 1H), 7.55-7.49 (m, 1H), 7.32-7.22 (m, 1H), 5.28-4.93 (m, 3H), 4.65-4.28 (m, 5 H), 4.25-3.95 (m, 2H), 3.88 (s, 3H), 3.73-3.38 (m, 2H), 3.24-3.06 (m, 1H), 3.06-2.87 (m, 4H), 2.45-2.33 (m, 4H), 2.06-1.65 (m, 2H), 1.20 (d, J=6.6, 2.6 Hz, 3H).

### Example 191:2-[(3R,4S)-3-Fluoro-1-((S)-2-hydroxy-propionyl)-piperidin-4-yloxy]-5-[2-(2-methoxy-1′-oxetan-3-yl-1′,2′,3′4′,5′,6′-hexahydro-[3,4′]bipyridinyl-6-ylamino)-pyrimidin-4-yl]-benzonitrile

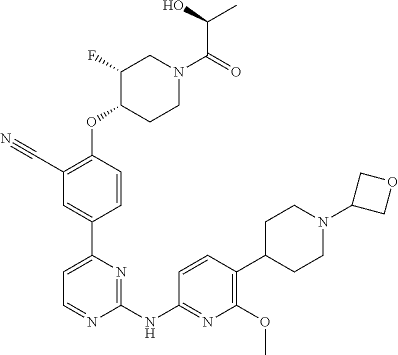 [SMILES: COc1nc(Nc2nccc(-c3ccc(O[C@H]4CCN(C(=O)[C@H](C)O)C[C@H]4F)c(C#N)c3)n2)ccc1C1CCN(C2COC2)CC1]

The title compound (37 mg) was synthesized using 2-((3R,4S)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (108.00 mg; 0.19 mmol; 1.00 eq.), (S)-2-Hydroxy-propionic acid (17.38 mg; 0.19 mmol; 1.00 eq 0-(7-Azabenzotriazol-1-yl)-N,N,N′,N′-tetramethyluronium hexaflurophosphate (HATU) (128.75 mg; 0.34 mmol; 1.75 eq.) and Ethyl-diisopropyl-amine (0.10 ml; 0.96 mmol; 5.00 eq.) in 31% yield, m/z: 632 (M+H). ¹H NMR (400 MHz, DMSO-d₆) δ 9.47 (s, 1H), 8.61 (d, J=5.8 Hz, 2H), 8.51 (d, J=8.9 Hz, 1H), 7.80 (d, J=8.0 Hz, 1H), 7.66-7.54 (m, 3H), 5.14 (d, J=19.8 Hz, 2H), 5.08-4.96 (m, 2H), 4.50 (dt, J=38.1, 6.3 Hz, 6H), 4.19 (d, J=9.5 Hz, 1H), 4.12-4.03 (m, 1H), 3.96 (d, J=13.0 Hz, 1H), 3.90 (s, 3H), 3.46-3.38 (m, 1H), 3.24 (s, 1H), 2.89 (s, 1H), 2.80 (d, J=10.7 Hz, 2H), 2.76-2.64 (m, 2H), 2.03-1.93 (m, 2H), 1.86 (t, J=11.2 Hz, 3H), 1.77-1.57 (m, 5H), 1.32-1.11 (m, 6H).

### Example 192: 2-[(3R,4R)-3-Fluoro-1-((S)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

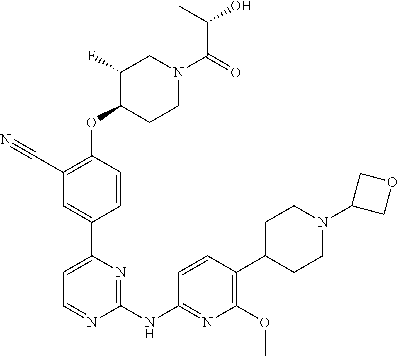 [SMILES: COc1nc(Nc2nccc(-c3ccc(O[C@@H]4CCN(C(=O)[C@H](C)O)C[C@H]4F)c(C#N)c3)n2)ccc1C1CCN(C2COC2)CC1]

The title compound (11 mg) was synthesized using 2-((3R,4R)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (200.00 mg; 0.36 mmol; 1.00 eq.), (S)-2-Hydroxy-propionic acid (32.19 mg; 0.36 mmol; 1.00 eq.), 0-(7-Azabenzotriazol-1-yl)-N,N,N′,N′-tetramethyluronium hexaflurophosphate (HATU) (238.43 mg; 0.63 mmol; 1.73 eq.) and Ethyl-diisopropyl-amine (0.18 ml; 1.79 mmol; 5.00 eq.) in 5% yield, m/z: 632 (M+H). ¹H NMR (400 MHz, DMSO-d6) δ 9.61 (s, 1H), 8.63 (d, J=5.8 Hz, 2H), 8.52 (d, J=9.2 Hz, 1H), 7.86 (d, J=8.1 Hz, 1H), 7.68-7.52 (m, 3H), 6.64 (s, 1H), 5.14 (s, 1H), 4.78 (d, J=6.5 Hz, 4H), 4.49 (s, 1H), 4.38 (s, 1H), 3.93 (s, 3H), 2.92 (s, 2H), 2.81 (s, 2H), 2.72 (d, J=11.7 Hz, 2H), 2.17 (s, 2H), 2.06-1.97 (m, 5H), 1.90 (t, J=12.8 Hz, 2H), 0.86 (t, J=6.7 Hz, 3H).

### Example 193: 2-[(3R,4R)-3-Fluoro-1-((R)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

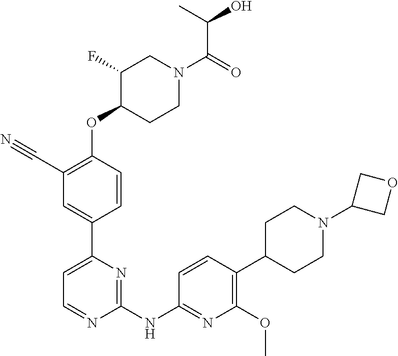 [SMILES: COc1nc(Nc2nccc(-c3ccc(O[C@@H]4CCN(C(=O)[C@@H](C)O)C[C@H]4F)c(C#N)c3)n2)ccc1C1CCN(C2COC2)CC1]

The title compound (53 mg) was synthesized using 2-((3R,4R)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (200.00 mg; 0.36 mmol; 1.00 eq.), (R)-2-Hydroxy-propionic acid (32.19 mg; 0.36 mmol; 1.00 eq.), 0-(7-Azabenzotriazol-1-yl)-N,N,N′,N′-tetramethyluronium hexaflurophosphate (HATU) (238.43 mg; 0.63 mmol; 1.75 eq.) and Ethyl-diisopropyl-amine (0.18 ml; 1.79 mmol; 5.00 eq.) in 24% yield, m/z: 632 (M+H). ¹H NMR (400 MHz, DMSO-d6) δ 10.36 (s, 1H), 9.61 (s, 1H), 8.63 (t, J=4.5 Hz, 2H), 8.52 (d, J=9.1 Hz, 1H), 7.85 (d, J=8.1 Hz, 1H), 7.68-7.52 (m, 3H), 5.12 (d, J=8.8 Hz, 1H), 4.78 (d, J=6.7 Hz, 5H), 4.64 (s, 1H), 4.49 (q, J=6.6 Hz, 1H), 4.42-4.35 (m, 1H), 4.16 (dd, J=27.5, 13.7 Hz, 1H), 3.93 (s, 3H), 3.81 (s, 1H), 3.01 (dd, J=18.8, 8.6 Hz, 3H), 2.17 (d, J=13.2 Hz, 1H), 2.02 (d, J=13.9 Hz, 2H), 1.92 (t, J=12.3 Hz, 2H), 1.83-1.77 (m, 1H), 1.31-1.19 (m, 2H), 1.23 (s, 3H).

### Example 194: 2-[(3S,4R)-3-Fluoro-1-((S)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

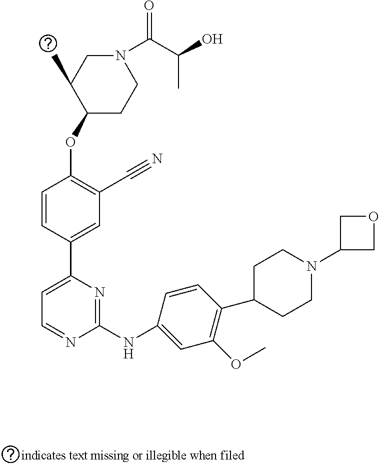 [SMILES: COc1cc(Nc2nccc(-c3ccc(O[C@@H]4CCN(C(=O)[C@H](C)O)C[C@@H]4C)c(C#N)c3)n2)ccc1C1CCN(C2COC2)CC1]

The title compound (13 mg) was synthesized using 2-((3S,4R)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (200.00 mg; 0.36 mmol; 1.00 eq.), (S)-2-Hydroxy-propionic acid (32.19 mg; 0.36 mmol; 1.00 eq.), 0-(7-Azabenzotriazol-1-yl)-N,N,N′,N′-tetramethyluronium hexaflurophosphate (HATU) (238.43 mg; 0.63 mmol; 1.75 eq.) and Ethyl-diisopropyl-amine (0.18 ml; 1.79 mmol; 5.00 eq.) in 6% yield, m/z: 632 (M+H). ¹H NMR (400 MHz, DMSO-d6) δ 10.48 (s, 1H), 9.61 (s, 1H), 8.66-8.59 (m, 2H), 8.51 (d, J=9.0 Hz, 1H), 7.85 (d, J=8.1 Hz, 1H), 7.67-7.51 (m, 3H), 5.19 (s, 1H), 5.16-5.07 (m, 1H), 4.97 (d, J=6.8 Hz, 1H), 4.78 (p, J=8.0 Hz, 4H), 4.50 (q, J=7.7, 7.0 Hz, 1H), 4.36 (dt, J=28.6, 8.7 Hz, 2H), 4.21 (s, 1H), 3.93 (s, 3H), 3.48 (s, 1H), 3.04 (t, J=7.9 Hz, 1H), 3.00 (s, 2H), 2.05-1.95 (m, 4H), 1.95-1.84 (m, 2H), 1.23 (d, J=6.5 Hz, 3H).

### Example 195: 2-[(3S,4R)-3-Fluoro-1-((R)-2-hydroxy-propionyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

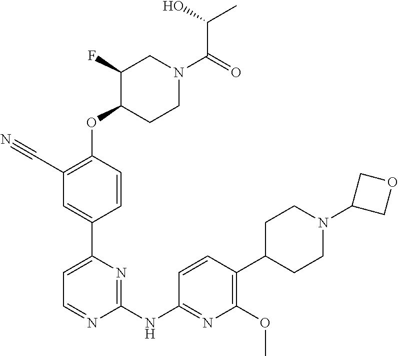 [SMILES: COc1nc(Nc2nccc(-c3ccc(O[C@@H]4CCN(C(=O)[C@@H](C)O)C[C@@H]4F)c(C#N)c3)n2)ccc1C1CCN(C2COC2)CC1]

The title compound (38 mg) was synthesized using 2-((3S,4R)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (200.00 mg; 0.36 mmol; 1.00 eq.), (R)-2-Hydroxy-propionic acid (32.19 mg; 0.36 mmol; 1.00 eq.), 0-(7-Azabenzotriazol-1-yl)-N,N,N′,N′-tetramethyluronium hexaflurophosphate (HATU) (238.43 mg; 0.63 mmol; 1.75 eq.) and Ethyl-diisopropyl-amine (0.18 ml; 1.79 mmol; 5.00 eq.) in 17% yield, m/z: 632 (M+H). ¹H NMR (400 MHz, DMSO-d6) δ 10.40 (s, 1H), 9.61 (s, 1H), 8.62 (t, J=4.7 Hz, 2H), 8.51 (dd, J=8.9, 2.2 Hz, 1H), 7.85 (d, J=8.0 Hz, 1H), 7.66-7.51 (m, 2H), 5.18 (d, J=8.5 Hz, 0H), 5.11 (s, 1H), 4.78 (d, J=6.8 Hz, 3H), 4.48 (dq, J=12.8, 6.5 Hz, 1H), 4.38 (p, J=7.6, 7.0 Hz, 1H), 4.27-3.95 (m, 1H), 3.93 (s, 2H), 3.46-3.30 (m, 1H), 3.21 (t, J=11.0 Hz, 1H), 3.10-2.94 (m, 2H), 2.51 (d, J=2.7 Hz, 2H), 2.01 (d, J=13.3 Hz, 3H), 1.98-1.86 (m, 1H), 1.86 (s, 1H), 1.22 (dd, J=6.7, 3.1 Hz, 3H).

### Example 196: 2-[(3S,4S)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

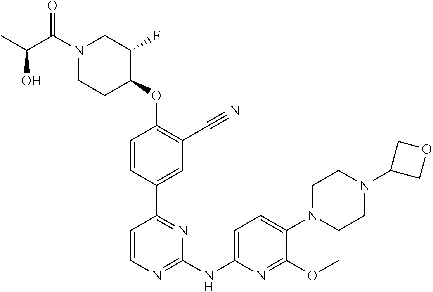 [SMILES: COc1nc(Nc2nccc(-c3ccc(O[C@H]4CCN(C(=O)[C@H](C)O)C[C@@H]4F)c(C#N)c3)n2)ccc1N1CCN(C2COC2)CC1]

The title compound (29.7 mg) was synthesized using 2-((3S,4S)-3-Fluoro-piperidin-4-yloxy)-5-{2-[6-methoxy-5-(4-oxetan-3-yl-piperazin-1-yl)-pyridin-2-ylamino]-pyrimidin-4-yl}-benzonitrile (80 mg) and (S)-2-Hydroxy-propionic acid (25.40 mg) using Method A in 32% yield, m/z: 633 (M+H). ¹H NMR (DMSO-d6): 9.36 (1H), 8.58 (2H), 8.47 (1H), 7.75 (1H), 7.67 (1H), 7.29 (1H), 5.12 (2H), 5.27 (1H), 4.77 (1H), 4.53 (2H), 4.48 (2H), 4.18 (1H), 3.91 (3H), 3.46 (3H), 2.98 (3H), 2.41 (3H), 2.13 (2H), 1.22 (3H).

### Example 197: 2-[(3S,4S)-1-((S)-2,3-Dihydroxy-propionyl)-3-fluoro-piperidin-4-yloxy]-5-{2 [6-methoxy-5-(4-oxetan-3-yl-piperazin-1-yl)-pyridin-2-ylamino]-pyrimidin-4-yl}-benzonitrile

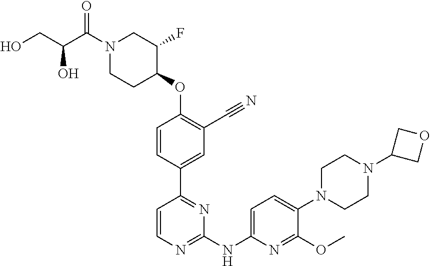 [SMILES: COc1nc(Nc2nccc(-c3ccc(O[C@H]4CCN(C(=O)[C@@H](O)CO)C[C@@H]4F)c(C#N)c3)n2)ccc1N1CCN(C2COC2)CC1]

The title compound (30.7 mg) was synthesized using 2-((3S,4S)-3-Fluoro-piperidin-4-yloxy)-5-{2-[6-methoxy-5-(4-oxetan-3-yl-piperazin-1-yl)-pyridin-2-ylamino]-pyrimidin-4-yl}-benzonitrile (80 mg) and (S)-2,3-Dihydroxy-propionic acid (32.40 mg) using Method A in 33% yield, m/z: 645 (M+H). ¹H NMR (DMSO-d6): 9.36 (1H), 8.58 (2H), 8.47 (1H), 7.75 (1H), 7.67 (1H), 7.29 (1H), 5.12 (2H), 5.27 (1H), 4.77 (1H), 4.53 (2H), 4.48 (2H), 4.18 (1H), 3.91 (3H), 3.46-3.52 (2H), 2.98 (3H), 2.41 (3H), 2.13 (2H).

### Example 198: 2-[[(3R,4S)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile and Example 199: 2-[[(3R,4S)-3-fluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile)

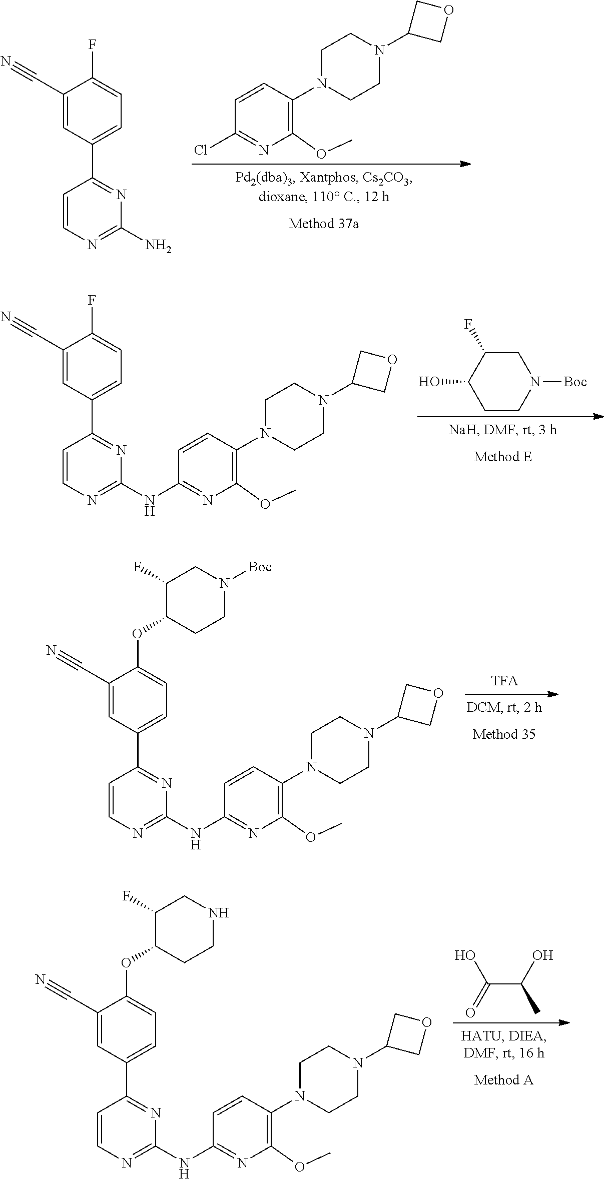

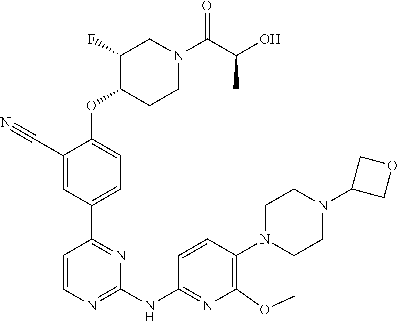 [SMILES: COc1nc(Nc2nccc(-c3ccc(O[C@H]4CCN(C(=O)[C@H](C)O)C[C@H]4F)c(C#N)c3)n2)ccc1N1CCN(C2COC2)CC1]

2-[[(3R,4S)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile: The title compound was prepared from 5-(2-aminopyrimidin-4-yl)-2-fluorobenzonitrile, 1-(6-chloro-2-methoxypyridin-3-yl)-4-(oxetan-3-yl)piperazine, and tert-butyl (3R,4S)-3-fluoro-4-hydroxypiperidine-1-carboxylate using Method 37a, E and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 20% to 45% gradient in 8 min; detector, UV 254 nm. 2-[[(3R,4S)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile was obtained as a light yellow solid (10 mg, 1.5% for 3 steps). HPLC: 97.7% purity, RT=3.38 min. MS: m/z=561.2[M+H]⁺. ¹H NMR (300 MHz, DMSO-d₆, ppm) δ 9.39 (s, 1H), 8.62-8.54 (m, 2H), 8.53-8.43 (m, 1H), 7.80-7.70 (m, 1H), 7.62-7.48 (m, 2H), 7.33-7.24 (m, 1H), 5.17-4.97 (m, 1H), 4.96-4.70 (m, 1H), 4.62-4.42 (m, 4H), 3.90 (s, 3H), 3.54-3.43 (m, 1H), 3.23-3.06 (m, 1H), 3.02-2.89 (m, 4H), 2.91-2.76 (m, 2H), 2.67-2.60 (m, 1H), 2.45-2.38 (m, 4H), 2.17-2.10 (m, 1H), 1.88-1.80 (m, 2H).

2-[[(3R,4S)-3-fluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile: The title compound was prepared from 2-[[(3R,4S)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 28% to 33% gradient in 8 min; detector, UV 254 nm. 2-[[(3R,4S)-3-fluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile was obtained as a light yellow solid (198 mg, 57%). HPLC: 98.1% purity, RT=8.50 min. MS: m/z=633.2 [M+H]⁺. ¹H NMR (300 MHz, DMSO-d₆, ppm) δ 9.41 (s, 1H), 8.64-8.45 (m, 3H), 7.79-7.70 (m, 1H), 7.68-7.58 (m, 1H), 7.58-7.50 (m, 1H), 7.32-7.23 (m, 1H), 5.17-4.94 (m, 3H), 4.65-4.31 (m, 5H), 4.24-4.03 (m, 2H), 3.90 (s, 3H), 3.75-3.39 (m, 2H), 3.30-3.10 (m, 1H), 3.01-2.95 (m, 4H), 2.44-2.37 (m, 4H), 2.09-1.73 (m, 2H), 1.26-1.16 (m, 3H).

### Example 201: 2-[[(3R,4S)-3-fluoro-1-[(2S)-2-hydroxypropanoyl]piperidin-4-yl]oxy]-5-(2-[[6-methoxy-5-(4-methylpiperazin-1-yl)pyridin-2-yl]amino]pyrimidin-4-yl)benzonitrile

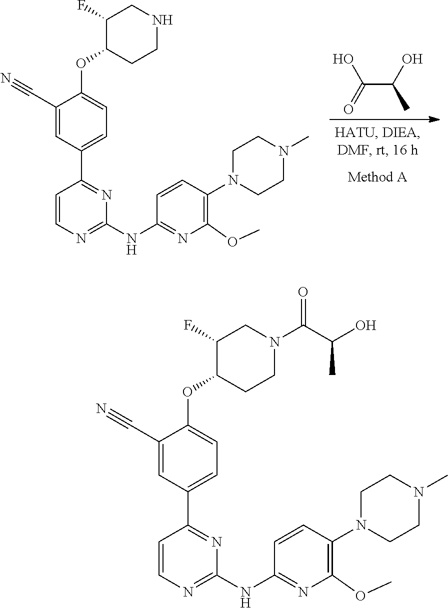

The title compound was prepared from 2-[[(3R,4S)-3-fluoropiperidin-4-yl]oxy]-5-(2-[[6-methoxy-5-(4-methylpiperazin-1-yl)pyridin-2-yl]amino]pyrimidin-4-yl)benzonitrile and (S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₁HCO₃ and 0.1% NH₃.H₂O), 28% to 58% gradient in 8 min; detector, UV 254 nm. 2-[[(3R,4S)-3-fluoro-1-[(2S)-2-hydroxypropanoyl]piperidin-4-yl]oxy]-5-(2-[[6-methoxy-5-(4-methylpiperazin-1-yl)pyridin-2-yl]amino]pyrimidin-4-yl)benzonitrile was obtained as an yellow solid (25 mg, 21%). HPLC: 99.3% purity, RT=3.93 min. MS: m/z=591.2[M+H]⁺. ¹H NMR (300 MHz, DMSO-d₆, ppm) δ 9.35 (s, 1H), 8.61-8.52 (m, 2H), 8.53-8.43 (m, 1H), 7.76-7.67 (m, 1H), 7.66-7.56 (m, 1H), 7.56-7.47 (m, 1H), 7.29-7.19 (m, 1H), 5.21-4.88 (m, 3H), 4.51-4.39 (m, 1H), 4.24-3.92 (m, 1H), 3.88 (s, 3H), 3.73-3.53 (m, 1H), 3.48-3.32 (m, 1H), 3.20-3.13 (m, 1H), 2.97-2.90 (m, 4H), 2.47-2.40 (m, 4H), 2.20 (s, 3H), 2.05-1.71 (m, 2H), 1.23 (d, J=6.5 Hz, 3H).

### Example 202: 2-[(3R,4R)-3-Fluoro-1-((S)-2-hydroxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

The title compound was prepared according to the procedures described in example 116 using 4-Chloro-pyrimidin-2-ylamine, (3R,4R)-4-[2-Cyano-4-(4,4,5,5-tetramethyl-[1,3,2]dioxaborolan-2-yl)-phenoxy]-3-fluoro-piperidine-1-carboxylic acid tert-butyl ester, 1-(6-Bromo-2-methoxy-pyridin-3-yl)-4-oxetan-3-yl-piperazine, and (S)-2-Hydroxy-propionic acid. HPLC: Purity 98%, RT: 2.00; MS: m/z=633.9 [M+H]⁺.

### Example 203: 2-[[1-(5-methyl-1H-1,2,4-triazole-3-carbonyl)piperidin-4-yl]oxy]-5-(2-[[4-(4-methylpiperazin-1-yl)phenyl]amino]pyrimidin-4-yl)benzonitrile

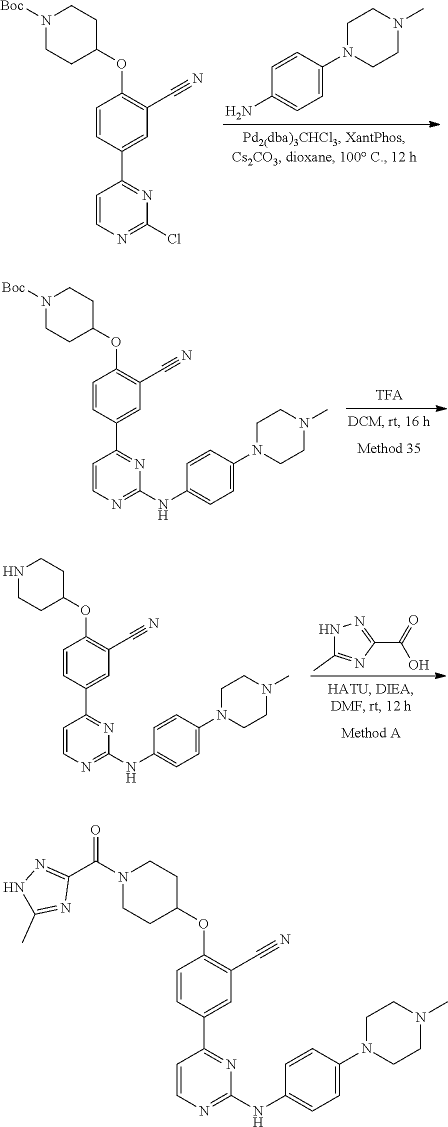

The title compound was prepared from tert-butyl 4-(4-(2-chloropyrimidin-4-yl)-2-cyanophenoxy)piperidine-1-carboxylate, 4-(4-methylpiperazin-1-yl)benzenamine and 5-methyl-1H-1,2,4-triazole-3-carboxylic acid using Method 37a, 35 and A. The final product was purified by prep-HPLC under the following conditions: column, XBridge Prep OBD C18 Column, 150×30 mm, 5 um; mobile phase, acetonitrile in water (with 10 mmol/L NH₁HCO₃ and 0.1% NH₃.H₂O), 20% to 50% gradient in 8 min; detector, UV 254 nm. 2-[[1-(5-methyl-1H-1,2,4-triazole-3-carbonyl)piperidin-4-yl]oxy]-5-(2-[[4-(4-methylpiperazin-1-yl)phenyl]amino]pyrimidin-4-yl)benzonitrile was obtained as an yellow solid (33 mg, 26% for 3 steps). HPLC: 99.5% purity, RT=3.96 min. MS: m/z=615.2 [M+H]⁺. ¹H NMR (300 MHz, DMSO-d₆, ppm) δ 14.02 (br s, 1H), 9.40 (s, 1H), 8.60-8.37 (m, 3H), 7.64-7.49 (m, 3H), 7.37 (d, J=5.2 Hz, 1H), 6.89 (d, J=9.1 Hz, 2H), 5.06-5.00 (m, 1H), 4.07-3.54 (m, 4H), 3.10-3.01 (m, 4H), 2.48-2.39 (m, 4H), 2.35 (s, 3H), 2.20 (s, 3H), 2.05-1.99 (m, 2H), 1.78-1.72 (m, 2H).

### Example 205: 2-[[3,3-difluoro-1-(2-methylpropanoyl)piperidin-4-yl]oxy]-5-[2-[(pyridin-3-yl)amino]pyrimidin-4-yl]benzonitrile

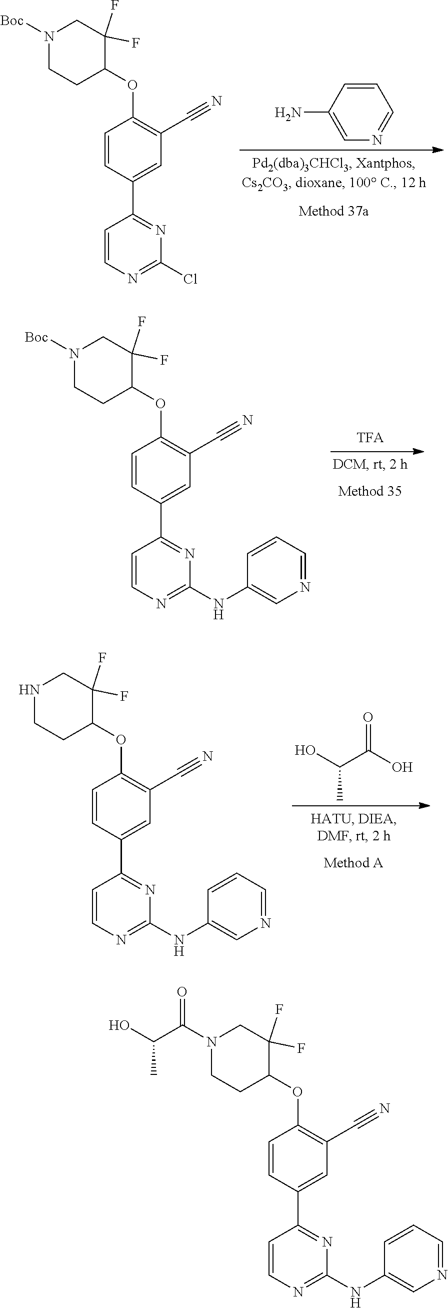

2-[(3,3-difluoropiperidin-4-yl)oxy]-5-[2-[(pyridin-3-yl)amino]pyrimidin-4-yl]benzonitrile: The title compound was prepared from tert-butyl 4-(4-(2-chloropyrimidin-4-yl)-2-cyanophenoxy)-3,3-difluoropiperidine-1-carboxylate and pyridin-3-amine using Method 37a and 35. The final product was purified by prep-HPLC under the following condition: column, XBridge Prep OBD C18, 30×150 mm, 5 um; mobile phase, acetonitrile in water (with 10 mmol/L NH₄HCO₃ and 0.1% NH₃.H₂O), 21% to 51% gradient in 8 min; detector, UV 254 nm. 2-[(3,3-difluoropiperidin-4-yl)oxy]-5-[2-[(pyridin-3-yl)amino]pyrimidin-4-yl]benzonitrile was obtained as brown solid (7 mg, 5% for 2 steps). HPLC: 98.5% purity, RT=2.30 min. MS: m/z=409.1 [M+H]⁺. ¹H NMR (300 MHz, DMSO-d₆, ppm) δ 9.88 (s, 1H), 8.98-8.90 (m, 1H), 8.63-8.50 (m, 2H), 8.50-8.40 (m, 1H), 8.27-8.13 (m, 2H), 7.68-7.58 (m, 1H), 7.57-7.49 (m, 1H), 7.39-7.28 (m, 1H), 5.25-5.16 (m, 1H), 3.19-3.12 (m, 1H), 3.03-2.75 (m, 2H), 2.74-2.67 (m, 1H), 2.58-2.49 (m, 1H), 2.06-1.99 (m, 1H), 1.87-1.80 (m, 1H).

2-[[3,3-difluoro-1-(2-methylpropanoyl)piperidin-4-yl]oxy]-5-[2-[(pyridin-3-yl)amino]pyrimidin-4-yl]benzonitrile: The title compound was prepared from 2-[(3,3-difluoropiperidin-4-yl)oxy]-5-[2-[(pyridin-3-yl)amino]pyrimidin-4-yl]benzonitrile and (2S)-2-hydroxypropanoic acid using Method A. The final product was purified by prep-HPLC under the following condition: column, Xselect CSH F-Phenyl OBD Column, 150×19 mm, 5 um; mobile phase, acetonitrile in water (with 10 mmol/L NH₁HCO₃ and 0.1% NH₃.H₂O), 10% to 30% gradient in 8 min; detector, UV 254 nm. 2-[[3,3-difluoro-1-(2-methylpropanoyl)piperidin-4-yl]oxy]-5-[2-[(pyridin-3-yl)amino]pyrimidin-4-yl]benzonitrile was obtained as white solid (29 mg, 19%). HPLC: 99.0% purity, RT=4.15 min. MS: m/z=481.1 [M+H]⁺. ¹H NMR (300 MHz, DMSO-de, ppm) δ 9.91 (s, 1H), 9.03-8.91 (m, 1H), 8.66-8.44 (m, 3H), 8.30-8.14 (m, 2H), 7.75-7.61 (m, 1H), 7.60-7.48 (m, 1H), 7.45-7.28 (m, 1H), 5.47-5.10 (m, 2H), 4.55-4.38 (m, 1H), 4.30-3.48 (m, 4H), 2.28-1.76 (m, 2H), 1.21 (d, J=6.4 Hz, 3H).

### Example 206: 2-([33-difluoro-1-[(2R)-2-hydroxypropanoyl]piperidin-4-yl]oxy)-5-[2-[(pyridin-3-yl)amino]pyrimidin-4-yl]benzonitrile

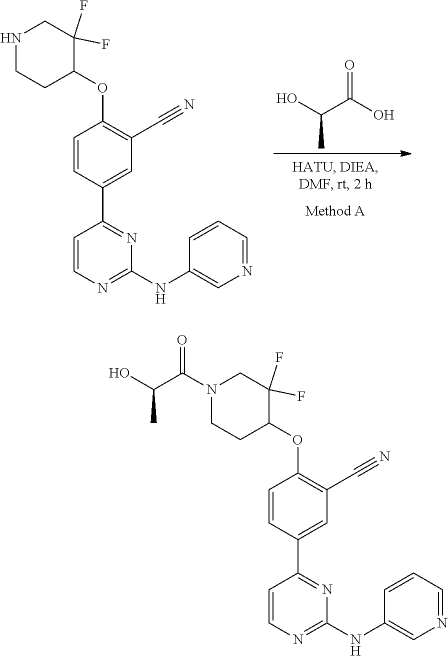

The title compound was prepared from 2-[(3,3-difluoropiperidin-4-yl)oxy]-5-[2-[(pyridin-3-yl)amino]pyrimidin-4-yl]benzonitrile and (2R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 15% to 35% gradient in 8 min; detector, UV 254 nm. 2-([3,3-difluoro-1-[(2R)-2-hydroxypropanoyl]piperidin-4-yl]oxy)-5-[2-[(pyridin-3-yl)amino]pyrimidin-4-yl]benzonitrile was obtained as white solid (28 mg, 20%). HPLC: 97.7% purity, RT=3.12 min. MS: m/z=481.1 [M+H]⁺. ¹H NMR (300 MHz, DMSO-d₆, ppm) δ 9.89 (s, 1H), 8.98-8.90 (m, 1H), 8.63-8.52 (m, 2H), 8.53-8.42 (m, 1H), 8.27-8.13 (m, 2H), 7.71-7.62 (m, 1H), 7.58-7.50 (m, 1H), 7.39-7.28 (m, 1H), 5.41-5.32 (m, 1H), 5.27-5.18 (m, 1H), 4.54-4.43 (m, 1H), 4.32-3.37 (m, 4H), 2.27-1.74 (m, 2H), 1.21 (d, J=6.5 Hz, 3H).

### Example 207: 2-([3,3-difluoro-1-[(2S)-2-hydroxypropanoyl]piperidin-4-yl]oxy)-5-[2-[(pyridin-4-yl)amino]pyrimidin-4-yl]benzonitrile

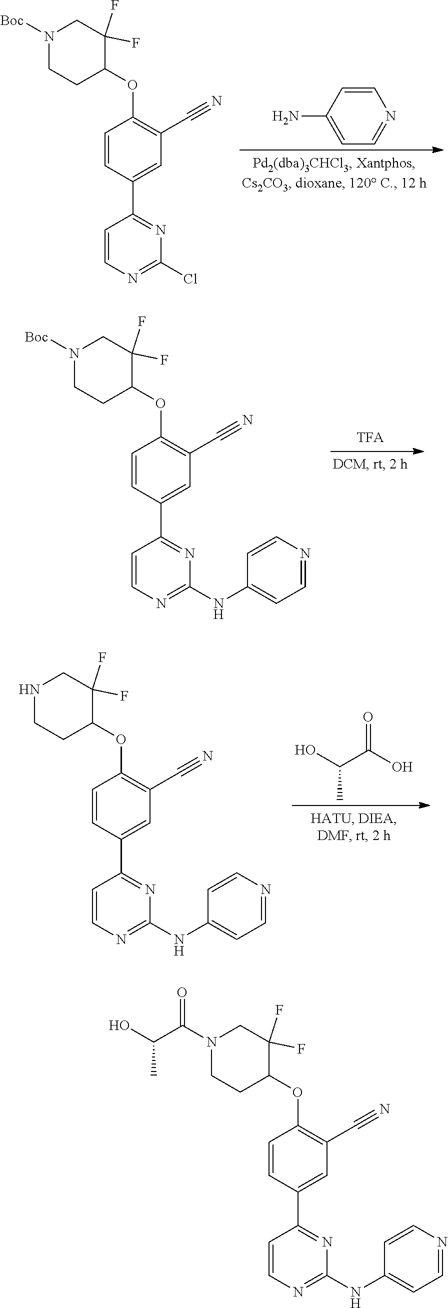

2-[(3,3-difluoropiperidin-4-yl)oxy]-5-[2-[(pyridin-4-yl)amino]pyrimidin-4-yl]benzonitrile: The title compound was prepared from tert-butyl 4-(4-(2-chloropyrimidin-4-yl)-2-cyanophenoxy)-3,3-difluoropiperidine-1-carboxylate and pyridin-4-amine using Method 37a and 35. The final product was purified by prep-HPLC under the following condition: column, XBridge Prep OBD C18, 30×150 mm, 5 um; mobile phase, acetonitrile in water (with 10 mmol/L NH₄HCO₃ and 0.1% NH₃.H₂O), 21% to 51% gradient in 8 min; detector, UV 254 nm. 2-[(3,3-difluoropiperidin-4-yl)oxy]-5-[2-[(pyridin-3-yl)amino]pyrimidin-4-yl]benzonitrile was obtained as brown solid (6 mg, 7.9% for 2 steps). HPLC: 99.3% purity, RT=3.18 min. MS: m/z=409.3 [M+H]⁺. ¹H NMR (300 MHz, DMSO-d₆, ppm) δ 10.17 (s, 1H), 8.70-8.61 (m, 1H), 8.61-8.53 (m, 1H), 8.53-8.43 (m, 1H), 8.41-8.32 (m, 2H), 7.83-7.74 (m, 2H), 7.69-7.58 (m, 2H), 5.32-5.11 (m, 1H), 3.20-3.05 (m, 1H), 3.03-2.78 (m, 2H), 2.74-2.67 (m, 1H), 2.60-2.48 (m, 1H), 2.06-2.00 (m, 1H), 1.88-1.79 (m, 1H).

2-([3,3-difluoro-1-[(2S)-2-hydroxypropanoyl]piperidin-4-yl]oxy)-5-[2-[(pyridin-4-yl)amino]pyrimidin-4-yl]benzonitrile: The title compound was prepared from 2-[(3,3-difluoropiperidin-4-yl)oxy]-5-[2-[(pyridin-4-yl)amino]pyrimidin-4-yl]benzonitrile and (2S)-2-hydroxypropanoic acid using Method A. The final product was purified by prep-HPLC under the following condition: column, Gemini-NX C18 AXAI Packed Column, 150×21.2 mm, 5 um; mobile phase, acetonitrile in water (with 10 mmol/L NH₄HCO₃ and 0.1% NH₃.H₂O), 10% to 40% gradient in 8 min; detector, UV 254 nm. 2-([3,3-difluoro-1-[(2S)-2-hydroxypropanoyl]piperidin-4-yl]oxy)-5-[2-[(pyridin-4-yl)amino]pyrimidin-4-yl]benzonitrile was obtained as white solid (27 mg, 14%). HPLC: 99.7% purity, RT=4.16 min. MS: m/z=481.1 [M+H]⁺. ¹H NMR (300 MHz, DMSO-d₆, ppm) δ 10.17 (s, 1H), 8.70-8.62 (m, 1H), 8.62-8.46 (m, 2H), 8.41-8.33 (m, 2H), 7.83-7.75 (m, 2H), 7.73-7.59 (m, 2H), 5.39-5.34 (m, 1H), 5.27-5.16 (m, 1H), 4.54-4.43 (m, 1H), 4.29-3.39 (m, 4H), 2.32-1.66 (m, 2H), 1.21 (d, J=6.4 Hz, 3H).

### Example 208: 2-([3,3-difluoro-1-[(2R)-2-hydroxypropanoyl]piperidin-4-yl]oxy)-5-[2-[(pyridin-4-yl)amino]pyrimidin-4-yl]benzonitrile

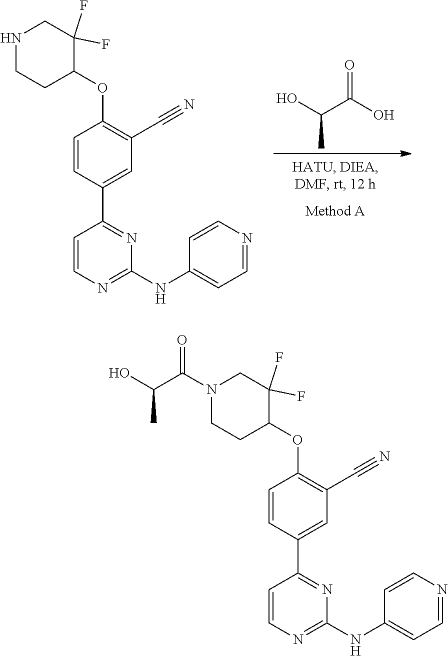

The title compound was prepared from 2-[(3,3-difluoropiperidin-4-yl)oxy]-5-[2-[(pyridin-4-yl)amino]pyrimidin-4-yl]benzonitrile and (2R)-2-hydroxypropanoic acid using Method A. The final product was purified by prep-HPLC under the following condition: column, Gemini-NX C18 AXAI Packed, 150×21.2 mm, 5 um; mobile phase, acetonitrile in water (with 10 mmol/L NH₄HCO₃ and 0.1% NH₃.H₂O), 10% to 40% gradient in 8 min; detector, UV 254 nm. 2-([3,3-difluoro-1-[(2R)-2-hydroxypropanoyl]piperidin-4-yl]oxy)-5-[2-[(pyridin-4-yl)amino]pyrimidin-4-yl]benzonitrile was obtained as white solid (30 mg, 23%). HPLC: HPLC: 99.7% purity, RT=3.14 min. MS: m/z=481.1 [M+H]⁺. ¹H NMR (300 MHz, DMSO-d₆, ppm) δ 10.17 (s, 1H), 8.71-8.62 (m, 1H), 8.63-8.46 (m, 2H), 8.41-8.33 (m, 2H), 7.83-7.75 (m, 2H), 7.73-7.59 (m, 2H), 5.41-5.35 (m, 1H), 5.27-5.17 (m, 1H), 4.54-4.43 (m, 1H), 4.33-3.38 (m, 4H), 2.25-1.73 (m, 2H), 1.21 (d, J=6.5 Hz, 3H).

### Example 209: 2-[3,3-Difluoro-1-((S)-2-hydroxy-propionyl)-piperidin-4-yloxy]-5-(2-phenylamino-pyrimidin-4-yl)-benzonitrile

### Intermediate: 2-(3,3-Difluoro-piperidin-4-yloxy)-5-(2-phenylamino-pyrimidin-4-yl)-benzonitrile hydrochloride

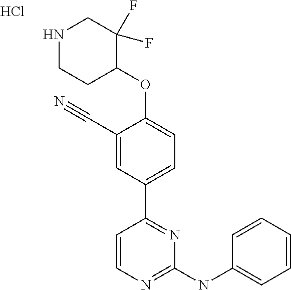 [SMILES: Cl ; N#Cc1cc(-c2ccnc(Nc3ccccc3)n2)ccc1OC1CCNCC1(F)F]

The title compound (800 mg) was synthesized from 4-[2-Cyano-4-(2-phenylamino-pyrimidin-4-yl)-phenoxy]-3,3-difluoro-piperidine-1-carboxylic acid tert-butyl ester (1200 mg) and HCl in dioxane (4M) using Method 17 in 75% yield, m/z: 408 (M+H). ¹H NMR (DMSO-d6): 8.62 (2H), 8.54 (1H), 7.77 (2H), 7.66 (1H), 7.50 (1H), 7.37 (2H), 7.00 (2H), 5.46 (1H), 3.74 (5H), 3.24 (2H), 2.38 (1H), 2.20 (1H).

### 2-[3,3-Difluoro-1-((S)-2-hydroxy-propionyl)-piperidin-4-yloxy]-5-(2-phenylamino-pyrimidin-4-yl)-benzonitrile

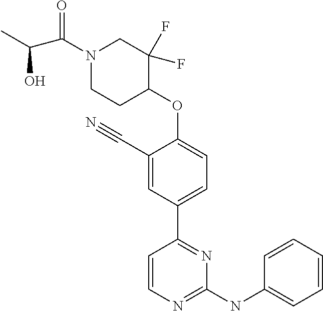 [SMILES: C[C@H](O)C(=O)N1CCC(Oc2ccc(-c3ccnc(Nc4ccccc4)n3)cc2C#N)C(F)(F)C1]

The title compound (44.4 mg) was synthesized using 2-(3,3-Difluoro-piperidin-4-yloxy)-5-(2-phenylamino-pyrimidin-4-yl)-benzonitrile hydrochloride (100 mg) and (S)-2-Hydroxy-propionic acid (40.60 mg) using Method A in 39% yield, m/z: 480 (M+H). ¹H NMR (DMSO-d6): 9.54 (1H), 8.62 (2H), 8.54 (1H), 7.81 (2H), 7.69 (1H), 7.50 (1H), 7.37 (2H), 6.99 (1H), 5.46 (1H), 5.23 (1H), 4.49 (1H), 3.75 (2H), 3.32 (2H), 2.18 (1H), 2.00 (1H). 1.23 (3H)

### Example 210: 2-[1-((S)-2,3-Dihydroxy-propionyl)-3,3-difluoro-piperidin-4-yloxy]-5-(2-phenylamino-pyrimidin-4-yl)-benzonitrile

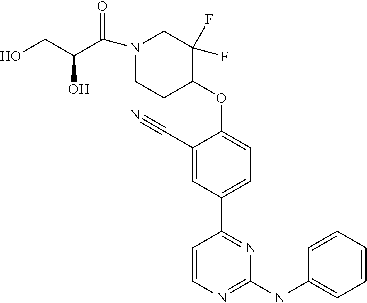 [SMILES: N#Cc1cc(-c2ccnc(Nc3ccccc3)n2)ccc1OC1CCN(C(=O)[C@@H](O)CO)CC1(F)F]

The title compound (36.5 mg) was synthesized using 2-(3,3-Difluoro-piperidin-4-yloxy)-5-(2-phenylamino-pyrimidin-4-yl)-benzonitrile hydrochloride (100 mg) and (S)-2,3-Dihydroxy-propionic acid (48.40 mg) using Method A in 32% yield, m/z: 496 (M+H). ¹H NMR (DMSO-d6): 10.4 (1H), 8.72 (2H), 6.64 (1H), 8.54 (1H), 8.41 (1H), 7.99 (2H), 7.76 (1H), 7.68 (1H), 5.39 (1H), 5.23 (1H), 4.49 (1H), 4.10 (1H), 4.06 (1H), 3.65 (1H), 2.18 (1H), 2.00 (1H).

### Example 211: 2-[3,3-Difluoro-1-((S)-2-hydroxy-propionyl)-piperidin-4-yloxy]-5-[2-(5,6-dimethoxy-pyridin-2-ylamino)-pyrimidin-4-yl]-benzonitrile

### Intermediate: 2-(3,3-Difluoro-piperidin-4-yloxy)-5-[2-(5,6-dimethoxy-pyridin-2-ylamino)-pyrimidin-4-yl]-benzonitrile hydrochloride

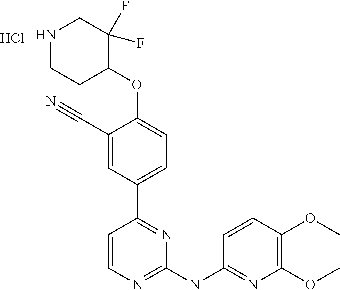 [SMILES: Cl ; COc1ccc(Nc2nccc(-c3ccc(OC4CCNCC4(F)F)c(C#N)c3)n2)nc1OC]

The title compound (1700 mg) was synthesized using 4-{2-Cyano-4-[2-(5,6-dimethoxy-pyridin-2-ylamino)-pyrimidin-4-yl]-phenoxy}-3,3-difluoro-piperidine-1-carboxylic acid tert-butyl ester (2200 mg) and HCl in dioxane (4M) using Method 17 in 86% yield, m/z: 469 (M+H). ¹H NMR (DMSO-d6): 8.62 (2H), 8.54 (1H), 7.66 (3H), 7.37 (1H), 5.46 (1H), 3.95 (3H), 3.74 (5H), 3.24 (2H), 2.38 (1H), 2.20 (1H).

### 2-[3,3-Difluoro-1-((S)-2-hydroxy-propionyl)-piperidin-4-yloxy]-5-[2-(5,6-dimethoxy-pyridin-2-ylamino)-pyrimidin-4-yl]-benzonitrile

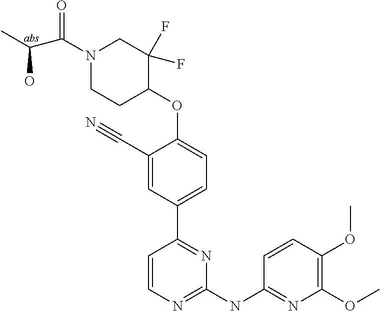 [SMILES: COc1ccc(Nc2nccc(-c3ccc(OC4CCN(C(=O)[C@H](C)O)CC4(F)F)c(C#N)c3)n2)nc1OC]

The title compound (86.2 mg) was synthesized using 2-(3,3-Difluoro-piperidin-4-yloxy)-5-[2-(5,6-dimethoxy-pyridin-2-ylamino)-pyrimidin-4-yl]-benzonitrile hydrochloride (100 mg) and (S)-2-Hydroxy-propionic acid (35.6 mg) using Method A in 78% yield, m/z: 541 (M+H). ¹H NMR (DMSO-d6): 9.34 (1H), 8.62 (2H), 8.54 (1H), 7.71 (1H), 7.66 (1H), 7.53 (1H), 7.37 (1H), 5.46 (1H), 5.23 (1H), 4.49 (1H), 3.95 (3H), 3.80 (3H), 2.18 (1H), 2.00 (1H). 1.23 (3H).

### Example 212: 2-([3,3-difluoro-1-[(2S)-2-hydroxypropanoyl]piperidin-4-yl]oxy)-5-[2-([4-[1-(oxetan-3-yl)piperidin-4-yl]phenyl]amino)pyrimidin-4-yl]benzonitrile

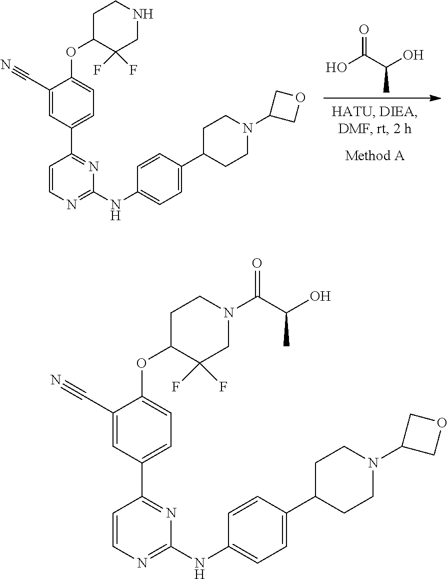

The title compound was prepared from 2-[(3,3-difluoropiperidin-4-yl)oxy]-5-[2-([4-[1-(oxetan-3-yl)piperidin-4-yl]phenyl]amino)pyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 32% to 42% gradient in 8 min; detector, UV 254 nm. 2-([3,3-difluoro-1-[(2S)-2-hydroxypropanoyl]piperidin-4-yl]oxy)-5-[2-([4-[1-(oxetan-3-yl)piperidin-4-yl]phenyl]amino)pyrimidin-4-yl]benzonitrile was obtained as a light yellow solid (25 mg, 25%). HPLC: 99.8% purity, RT=4.55 min. MS: m/z=619.2 [M+H]⁺. ¹H NMR (300 MHz, DMSO-d6, ppm) δ9.61 (s, 1H), 8.58-8.42 (m, 3H), 7.73-7.61 (m, 3H), 7.49-7.41 (m, 1H), 7.22-7.13 (m, 2H), 5.39-5.33 (m, 1H), 5.26-5.18 (m, 1H), 4.58-4.38 (m, 5H), 4.30-3.47 (m, 3H), 3.43-3.33 (m, 1H), 2.83-2.73 (m, 2H), 2.47-0.38 (m, 1H), 2.25-1.91 (m, 2H), 1.91-1.54 (m, 6H), 1.21 (d, 7=6.5 Hz, 3H).

### Example 213: 2-([3,3-difluoro-1-[(2S)-2-hydroxypropanoyl]piperidin-4-yl]oxy)-5-[2-([4-[1-(oxetan-3-yl)pyrrolidin-3-yl]phenyl]amino)pyrimidin-4-yl]benzonitrile

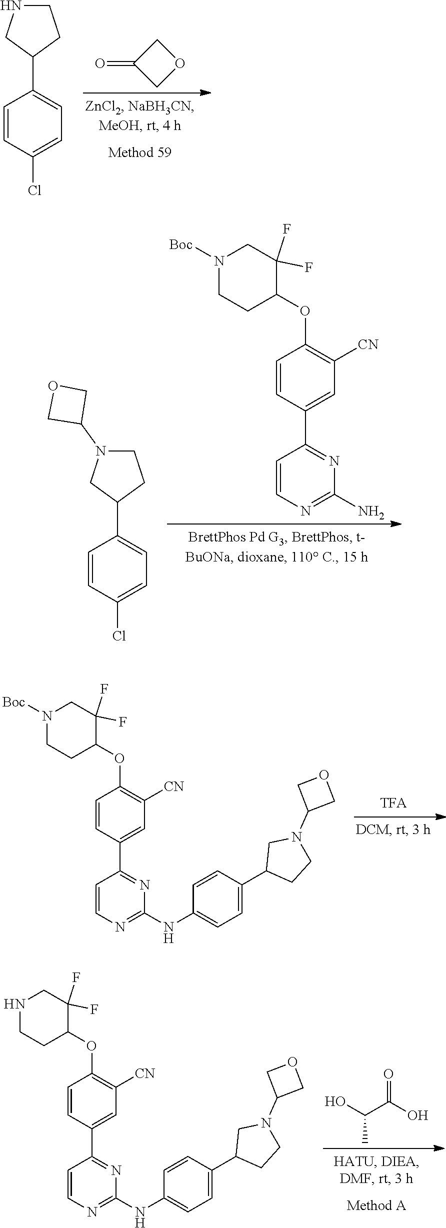

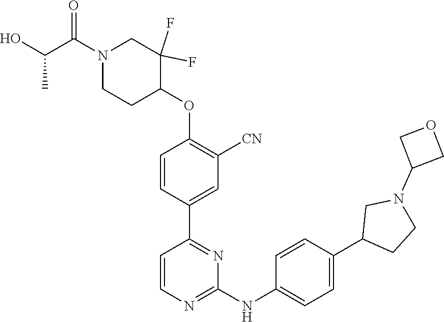 [SMILES: C[C@H](O)C(=O)N1CCC(Oc2ccc(-c3ccnc(Nc4ccc(C5CCN(C6COC6)C5)cc4)n3)cc2C#N)C(F)(F)C1]

The title compound was prepared from oxetan-3-one, 3-(4-chlorophenyl)pyrrolidine, tert-butyl 4-(4-(2-aminopyrimidin-4-yl)-2-cyanophenoxy)-3,3-difluoropiperidine-1-carboxylate and (S)-2-hydroxypropanoic acid using Method 59, 45, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃), 30% to 60% gradient in 8 min; detector, UV 254 nm. 2-([3,3-difluoro-1-[(2S)-2-hydroxypropanoyl]piperidin-4-yl]oxy)-5-[2-([4-[1-(oxetan-3-yl)pyrrolidin-3-yl]phenyl]amino)pyrimidin-4-yl]benzonitrile was obtained as an yellow solid (26 mg, 9% for 4 steps). HPLC: 95.9% purity, RT=4.51 min. MS: m/z=605.2 [M+H]1H NMR (300 MHz, DMSO-d6, ppm) δ 9.60 (s, 1H), 8.58-8.43 (m, 3H), 7.74-7.61 (m, 3H), 7.45 (d, J=5.2 Hz, 1H), 7.22 (d, J=8.4 Hz, 2H), 5.39-5.33 (m, 1H), 5.23-5.17 (m, 1H), 4.62-4.42 (m, 5H), 4.30-3.53 (m, 5H), 3.30-3.22 (m, 1H), 2.96-2.85 (m, 1H), 2.71-2.54 (m, 2H), 2.44-2.32 (m, 1H), 2.30-1.69 (m, 4H), 1.21 (d, J=6.5 Hz, 3H).

### Example 214: 2-([3,3-difluoro-1-[(2R)-2-hydroxypropanoyl]piperidin-4-yl]oxy)-5-[2-([4-[1-(oxetan-3-yl)pyrrolidin-3-yl]phenyl]amino)pyrimidin-4-yl]benzonitrile

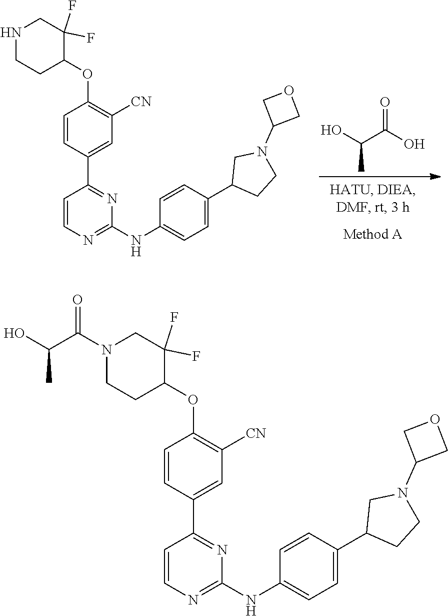

The title compound was prepared from 2-(3,3-difluoropiperidin-4-yloxy)-5-(2-(4-(1-(oxetan-3-yl)pyrrolidin-3-yl)phenylamino)pyrimidin-4-yl)benzonitrile and (R)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₁HCO₃), 20% to 50% gradient in 8 min; detector, UV 254 nm. 2-([3,3-difluoro-1-[(2S)-2-hydroxypropanoyl]piperidin-4-yl]oxy)-5-[2-([4-[1-(oxetan-3-yl)pyrrolidin-3-yl]phenyl]amino)pyrimidin-4-yl]benzonitrile was obtained as an yellow solid (26 mg, 27%). HPLC: 97.5% purity, RT=4.51 min. MS: m/z=605.2 [M+H]⁺. ¹H NMR (300 MHz, DMSO-d6, ppm) δ 9.60 (s, 1H), 8.58-8.49 (m, 2H), 8.53-8.43 (m, 1H), 7.74-7.61 (m, 3H), 7.45 (d, J=5.2 Hz, 1H), 7.22 (d, J=8.5 Hz, 2H), 5.39-5.33 (m, 1H), 5.25-5.17 (m, 1H), 4.66-4.40 (m, 5H), 4.31-3.50 (m, 5H), 3.30-3.23 (m, 1H), 2.90 (t, J=8.3 Hz, 1H), 2.71-2.57 (m, 2H), 2.38 (t, J=8.3 Hz, 1H), 2.31-1.66 (m, 4H), 1.21 (d, 7=6.5 Hz, 3H).

### Example 215: 2-([3,3-difluoro-1-[(2R)-2-hydroxypropanoyl]piperidin-4-yl]oxy)-5-(2-[[4-(morpholin-4-yl)phenyl]amino]pyrimidin-4-yl)benzonitrile

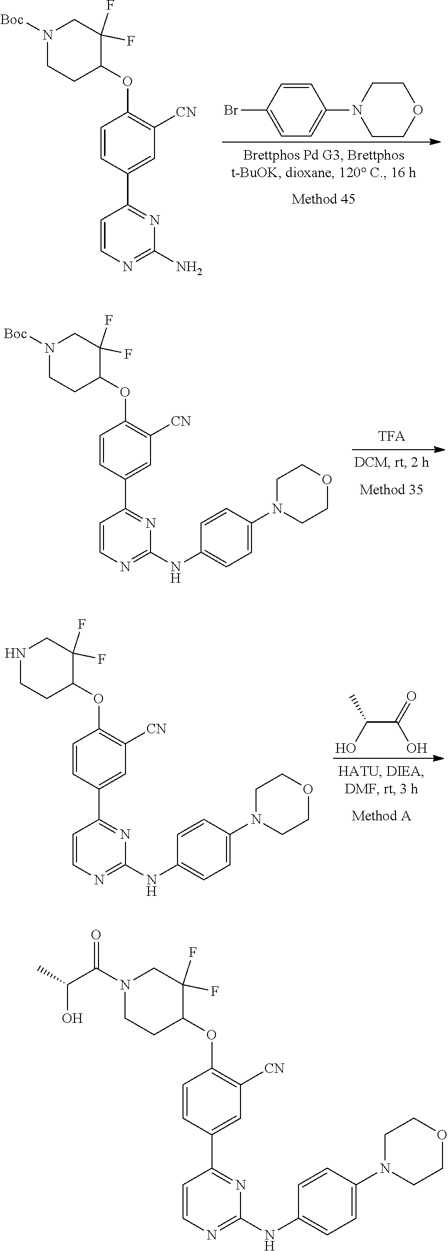

2-[(3,3-difluoropiperidin-4-yl)oxy]-5-(2-[[4-(morpholin-4-yl)phenyl]amino]pyrimidin-4-yl)benzonitrile: The title compound was prepared from tert-butyl 4-(4-(2-aminopyrimidin-4-yl)-2-cyanophenoxy)-3,3-difluoropiperidine-1-carboxylate and 4-(4-bromophenyl)morpholine using Method 45 and 35. The final product was purified by prep-HPLC under the following conditions: column, XBridge Prep OBD C18 Column, 150×30 mm, 5 um; mobile phase, acetonitrile in water (with 10 mmol/L NH₄HCO₃+0.1% NH₃.H₂O), 27% to 47% gradient in 8 min; detector, UV 254 nm. 2-[(3,3-difluoropiperidin-4-yl)oxy]-5-(2-[[4-(morpholin-4-yl)phenyl]amino]pyrimidin-4-yl)benzonitrile was obtained as an yellow solid (4 mg, 19% for 2 steps). HPLC: 97.5% purity, RT=3.03 min. MS: m/z=493.2 [M+H]⁺. ¹H NMR (300 MHz, DMSO-d6, ppm) δ9.44 (s, 1H), 8.54-8.44 (m, 2H), 8.48-8.38 (m, 1H), 7.66-7.56 (m, 3H), 7.42-7.35 (m, 1H), 6.95-6.86 (m, 2H), 5.22-5.16 (m, 1H), 3.77-3.68 (m, 4H), 3.25-2.63 (m, 8H), 2.20-1.70 (m, 2H).

2-([3,3-difluoro-1-[(2R)-2-hydroxypropanoyl]piperidin-4-yl]oxy)-5-(2-[[4-(morpholin-4-yl)phenyl]amino]pyrimidin-4-yl)benzonitrile: The title compound was prepared from tert-butyl 4-(4-(2-aminopyrimidin-4-yl)-2-cyanophenoxy)-3,3-difluoropiperidine-1-carboxylate, 4-(4-bromophenyl)morpholine and (R)-2-hydroxypropanoic acid using Methods 45, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃), 35% to 65% gradient in 8 min; detector, UV 254 nm. 2-([3,3-difluoro-1-[(2R)-2-hydroxypropanoyl]piperidin-4-yl]oxy)-5-(2-[[4-(morpholin-4-yl)phenyl]amino]pyrimidin-4-yl)benzonitrile was obtained as an yellow solid (26 mg, 20% for 3 steps). HPLC: 98.6% purity, RT=4.86 min. MS: m/z=565.2 [M+H]⁺. ¹H NMR (300 MHz, DMSO-d6, ppm) δ9.45 (s, 1H), 8.57-8.41 (m, 3H), 7.69-7.57 (m, 3H), 7.39 (d, J=5.2 Hz, 1H), 6.95-6.86 (m, 2H), 5.38-5.32 (m, 1H), 5.27-5.17 (m, 1H), 4.54-4.44 (m, 1H), 4.28-3.44 (m, 8H), 3.08-2.98 (m, 4H), 2.33-1.73 (m, 2H), 1.21 (d, J=6.5 Hz, 3H).

### Example 216: 2-([3,3-difluoro-1-[(2S)-2-hydroxypropanoyl]piperidin-4-yl]oxy)-5-(2-[[4-(morpholin-4-yl)phenyl]amino]pyrimidin-4-yl)benzonitrile

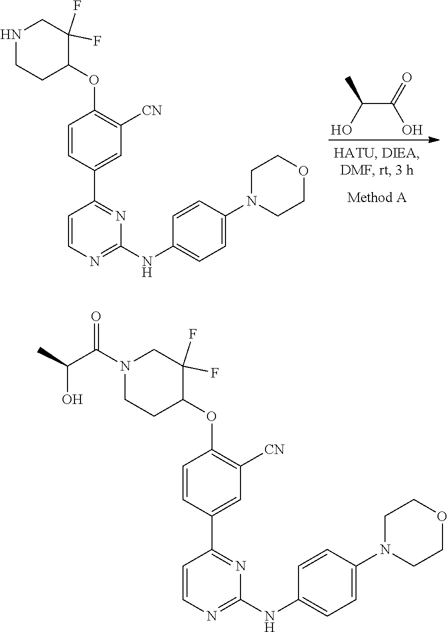

The title compound was prepared from 2-(3,3-difluoropiperidin-4-yloxy)-5-(2-(4-morpholinophenylamino)pyrimidin-4-yl)benzonitrile and (S)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₄HCO₃), 25% to 45% gradient in 8 min; detector, UV 254 nm. 2-([3,3-difluoro-1-[(2S)-2-hydroxypropanoyl]piperidin-4-yl]oxy)-5-(2-[[4-(morpholin-4-yl)phenyl]amino]pyrimidin-4-yl)benzonitrile was obtained as an yellow solid (29 mg, 28%). HPLC: 96.5% purity, RT=7.38 min. MS: m/z=566.2 [M+H]⁺. ¹H NMR (300 MHz, DMSO-d₆. ppm) δ 9.47 (s, 1H), 8.59-8.43 (m, 3H), 7.71-7.58 (m, 3H), 7.41 (d, J=5.2 Hz, 1H), 6.99-6.87 (m, 2H), 5.42-5.32 (m, 1H), 5.29-5.19 (m, 1H), 4.56-4.45 (m, 1H), 4.29-3.55 (m, 8H), 3.10-3.00 (m, 4H), 2.16-1.85 (m, 2H), 1.23 (d, J=6.4 Hz, 3H).

### Example 217: 2-([3,3-difluoro-1-[(2S)-2-hydroxypropanoyl]piperidin-4-yl]oxy)-5-[2-([3-methoxy-4-[4-(oxetan-3-yl)piperazin-1-yl]phenyl]amino)pyrimidin-4-yl]benzonitrile

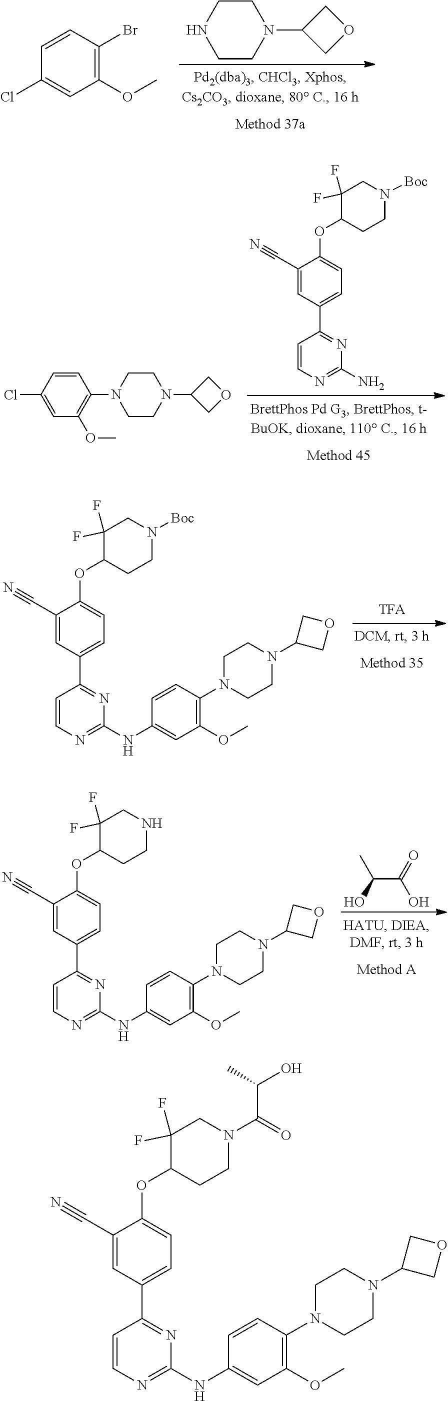

The title compound was prepared from 1-bromo-4-chloro-2-methoxybenzene, 1-(oxetan-3-yl)piperazine, tert-butyl 4-(4-(2-aminopyrimidin-4-yl)-2-cyanophenoxy)-3,3-difluoropiperidine-1-carboxylate and (S)-2-hydroxypropanoic acid using Method 37a, 45, 35 and A. The final product was purified by prep-HPLC under the following conditions: column, XBridge Prep OBD C18 Column, 150×19 mm, 5 um; mobile phase, EtOH in water (with 10 mmol/L NR₄HCO₃), 30% to 40% gradient in 8 min; detector, UV 254 nm. 2-([3,3-difluoro-1-[(2S)-2-hydroxypropanoyl]piperidin-4-yl]oxy)-5-[2-([3-methoxy-4-[4-(oxetan-3-yl)piperazin-1-yl]phenyl]amino)pyrimidin-4-yl]benzonitrile was obtained as an yellow solid (18 mg, 4% for 4 steps). HPLC: 97.3% purity, RT=4.45 min. MS: m/z=650.2 [M+H]⁺. ¹H NMR (300 MHz, DMSO-d6, ppm) δ9.54 (s, 1H), 8.61-8.43 (m, 3H), 7.69-7.57 (m, 2H), 7.43 (d, J=5.2 Hz, 1H), 7.26-7.17 (m, 1H), 6.84 (d, J=8.6 Hz, 1H), 5.40-5.34 (m, 1H), 5.27-5.17 (m, 1H), 4.62-4.38 (m, 5H), 4.30-3.83 (m, 2H), 3.80 (s, 3H), 3.72-3.56 (m, 2H), 3.51-3.42 (m, 1H), 2.97-2.91 (m, 4H), 2.42-2.36 (m, 4H), 2.24-1.82 (m, 2H), 1.21 (d, J=6.5 Hz, 3H).

### Example 218: 5-[2-[(6-methoxypyridin-2-yl)amino]pyrimidin-4-yl]-2-[[1-(5-methyl-1H-1,2,4-triazole-3-carbonyl)piperidin-4-yl]oxy]benzonitrile

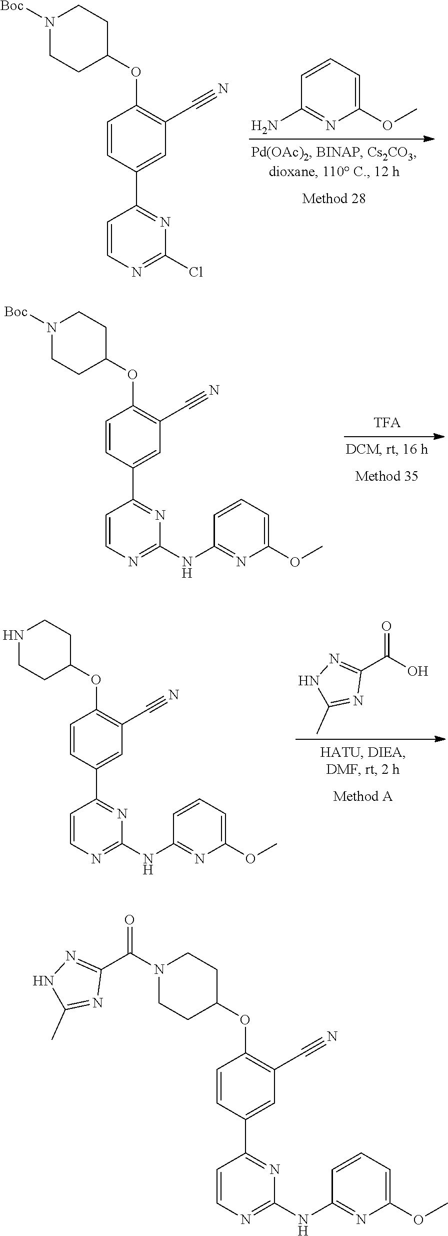

The title compound was prepared from tert-butyl 4-(4-(2-chloropyrimidin-4-yl)-2-cyanophenoxy)piperidine-1-carboxylate, 6-methoxypyridin-2-amine and 5-methyl-1H-1,2,4-triazole-3-carboxylic acid using Method 28, 35 and A. The final product was purified by prep-HPLC under the following conditions: column, XBridge Prep OBD C18 Column, 150×30 mm, 5 um; mobile phase, acetonitrile in water (with 10 mmol/L NH₁HCO₃ and 0.1% NH₃.H₂O), 30% to 60% gradient in 8 min; detector, UV 254 nm. 5-[2-[(6-methoxypyridin-2-yl)amino]pyrimidin-4-yl]-2-[[1-(5-methyl-1H-1,2,4-triazole-3-carbonyl)piperidin-4-yl]oxy]benzonitrile was obtained as an yellow solid (32 mg, 2% for 3 steps). HPLC: 98.2% purity, RT=4.71 min. MS: m/z=512.2 [M+H]⁺. ¹H NMR (300 MHz, DMSO-d₆, ppm) δ 14.00 (br s, 1H), 9.58 (s, 1H), 8.65-8.56 (m, 2H), 8.54-8.44 (m, 1H), 7.89-7.81 (m, 1H), 7.73-7.62 (m, 1H), 7.62-7.52 (m, 2H), 6.41 (d, J=7.9 Hz, 1H), 5.05 (s, 1H), 4.16-3.54 (m, 7H), 2.36 (s, 3H), 2.06-2.00 (m, 2H), 1.79-1.73 (m, 2H).

### Example 219: 2-(3,3-difluoro-1-((S)-2-hydroxypropanoyl)piperidin-4-yloxy)-5-(2-(pyridin-2-ylamino)pyrimidin-4-yl)benzonitrile

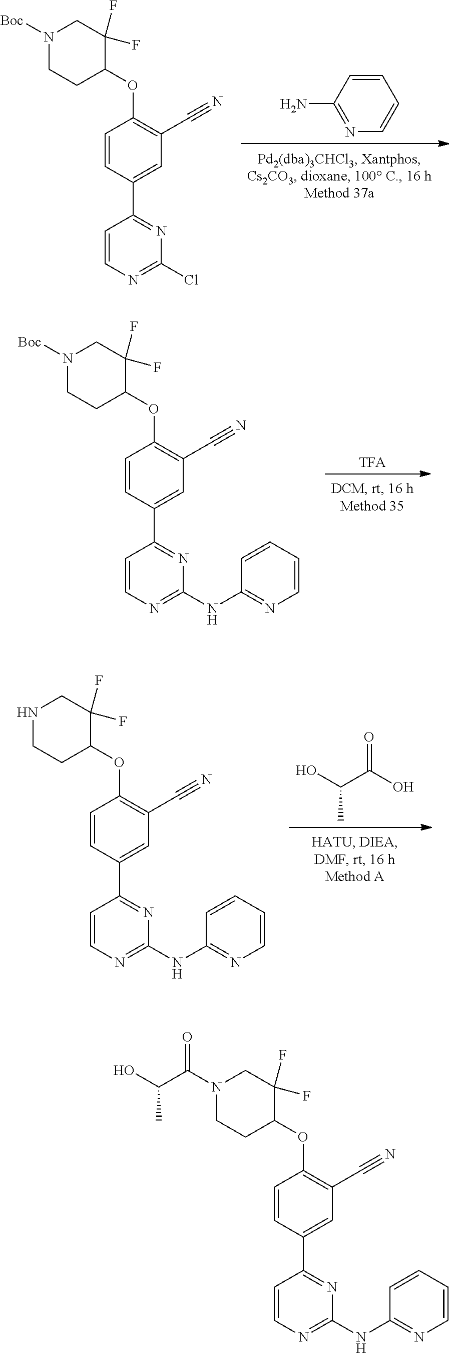

2-[(3,3-difluoropiperidin-4-yl)oxy]-5-[2-[(pyridin-2-yl)amino]pyrimidin-4-yl]benzonitrile: The title compound was prepared from tert-butyl 4-(4-(2-chloropyrimidin-4-yl)-2-cyanophenoxy)-3,3-difluoropiperidine-1-carboxylate and pyridin-2-amine using Method 37a and 35. The final product was purified by prep-HPLC under the following condition: column, Gemini-NX C18 AXAI Packed, 21.2×150 mm, 5 um; mobile phase, acetonitrile in water (with 10 mmol/L NH₄HCO₃ and 0.1% NH₃.H₂O), 22% to 50% gradient in 8 min; detector, UV 254 nm. 2-[(3,3-difluoropiperidin-4-yl)oxy]-5-[2-[(pyridin-2-yl)amino]pyrimidin-4-yl]benzonitrile was obtained as white solid (7 mg, 18% for 2 steps). HPLC: 99.9% purity, RT=2.38 min. MS: m/z=409.1 [M+H]⁺. ¹H NMR (300 MHz, DMSO-d₆, ppm) δ 9.87 (s, 1H), 8.66-8.54 (m, 2H), 8.54-8.44 (m, 1H), 8.34-8.24 (m, 2H), 7.84-7.71 (m, 1H), 7.68-7.54 (m, 2H), 7.05-6.94 (m, 1H), 5.25-5.18 (m, 1H), 3.24-3.05 (m, 1H), 3.04-2.79 (m, 2H), 2.79-2.62 (m, 1H), 2.61-2.48 (m, 1H), 2.06-2.00 (m, 1H), 1.88-1.78 (m, 1H).

2-([3,3-difluoro-1-[(2S)-2-hydroxypropanoyl]piperidin-4-yl]oxy)-5-[2-[(pyridin-2-yl)amino]pyrimidin-4-yl]benzonitrile: The title compound was prepared from 2-(3,3-difluoropiperidin-4-yloxy)-5-(2-(pyridin-2-ylamino)pyrimidin-4-yl)benzonitrile and (S)-2-hydroxypropanoic acid using Method A. The final product was purified by prep-HPLC under the following condition: column, Xselect Peptide CSH Column, 150×19 mm, 5 um; mobile phase, acetonitrile in water (with 10 mmol/L NH₄HCO₃ and 0.1% NH₃.H₂O), 34% to 42% gradient in 8 min; detector, UV 254 nm. 2-([3,3-difluoro-1-[(2S)-2-hydroxypropanoyl]piperidin-4-yl]oxy)-5-[2-([3-methoxy-5-[4-(oxetan-3-yl)piperazin-1-yl]pyridin-2-yl]amino)pyrimidin-4-yl]benzonitrile was obtained as white solid (26 mg, 19%). HPLC: 97.8% purity, RT=4.23 min. MS: m/z=480.9 [M+H]⁺. ¹H NMR (300 MHz, DMSO-d₆, ppm) δ 9.92-9.85 (m, 1H), 8.67-8.56 (m, 2H), 8.57-8.47 (m, 1H), 8.34-8.24 (m, 2H), 7.84-7.67 (m, 1H), 7.73-7.56 (m, 2H), 7.06-6.95 (m, 1H), 5.40-5.35 (m, 1H), 5.27-5.17 (m, 1H), 4.54-4.43 (m, 1H), 4.32-3.43 (m, 4H), 2.27-1.71 (m, 2H), 1.21 (d, J=6.4 Hz, 3H).

### Example 220: 2-[3,3-Difluoro-1-(2-hydroxy-acetyl)-piperidin-4-yloxy]-5-[2-(pyridin-2-ylamino)-pyrimidin-4-yl]-benzonitrile

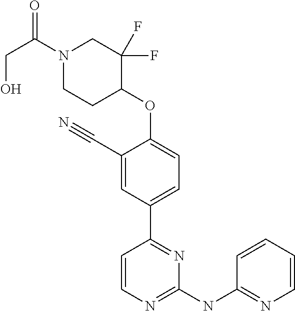 [SMILES: N#Cc1cc(-c2ccnc(Nc3ccccn3)n2)ccc1OC1CCN(C(=O)CO)CC1(F)F]

The title compound (16.10 mg) was synthesized using 2-(3,3-Difluoro-piperidin-4-yloxy)-5-[2-(pyridin-2-ylamino)-pyrimidin-4-yl]-benzonitrile hydrochloride (100 mg) and hydroxy-acetic acid (34.19 mg) using Method A in 15% yield, m/z: 467 (M+H). ¹H NMR (DMSO-d6): 9.84 (1H), 8.57 (2H), 8.51 (1H), 8.31 (2H), 7.79 (1H), 7.67 (1H), 7.63 (1H), 7.01 (1H), 5.37 (1H), 4.86 (1H), 4.17 (2H), 4.07 (1H), 3.89 (2H), 3.61 (1H), 3.51 (1H), 2.51 (1H), 2.01 (1H), 1.89 (1H).

### Example 221: 2-[3,3-Difluoro-1-((R)-2-hydroxy-propionyl)-piperidin-4-yloxy]-5-[2-(pyridin-2-ylamino)-pyrimidin-4-yl]-benzonitrile

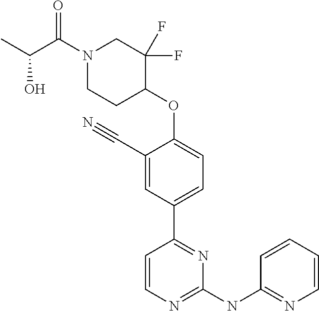 [SMILES: C[C@@H](O)C(=O)N1CCC(Oc2ccc(-c3ccnc(Nc4ccccn4)n3)cc2C#N)C(F)(F)C1]

The title compound (36.7 mg) was synthesized using 2-(3,3-Difluoro-piperidin-4-yloxy)-5-[2-(pyridin-2-ylamino)-pyrimidin-4-yl]-benzonitrile hydrochloride (100 mg) and (R)-2-Hydroxy-propionic acid (40.50 mg) using Method A in 33% yield, m/z: 481 (M+H). ¹H NMR (DMSO-d6): 9.84 (1H), 8.57 (2H), 8.51 (1H), 8.31 (2H), 7.79 (1H), 7.67 (1H), 7.63 (1H), 7.01 (1H), 5.37 (1H), 4.86 (1H), 4.17 (2H), 4.07 (1H), 3.89 (1H), 3.61 (1H), 3.51 (1H), 2.51 (1H), 2.01 (1H), 1.89 (1H), 1.22 (3H).

### Example 222: 2-(3,3-Difluoro-piperidin-4-yloxy)-5-{2-[3-(1-oxetan-3-yl-piperidin-4-yl)-phenylamino]-pyrimidin-4-yl}-benzonitrile

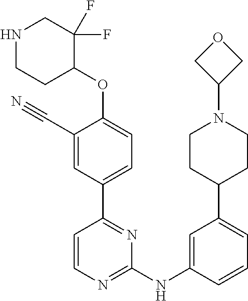 [SMILES: N#Cc1cc(-c2ccnc(Nc3cccc(C4CCN(C5COC5)CC4)c3)n2)ccc1OC1CCNCC1(F)F]

The title compound (280 mg) was synthesized using 4-(2-Cyano-4-{2-[3-(1-oxetan-3-yl-piperidin-4-yl)-phenylamino]-pyrimidin-4-yl}-phenoxy)-3,3-difluoro-piperidine-1-carboxylic acid tert-butyl ester (680 mg) with TFA (5 mL) in 48% yield, m/z: 547 (M+H). ¹H NMR (DMSO-d6): 9.62 (1H), 8.57 (2H), 8.49 (1H), 7.83 (1H), 7.62 (1H), 7.53 (2H), 7.23 (1H), 6.86 (1H), 5.26 (1H), 4.57 (1H0, 4.46 (1H), 3.39 (1H), 3.18 (1H), 2.84 (3H), 2.69 (1H), 2.03 (1H), 1.85 (4H), 1.68 (2H).

### Example 223: 2-[3,3-Difluoro-1-(2-hydroxy-acetyl)-piperidin-4-yloxy]-5-{2-[3-(1-oxetan-3-yl-piperidin-4-yl)-phenylamino]-pyrimidin-4-yl}-benzonitrile

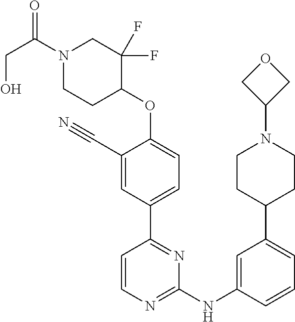 [SMILES: N#Cc1cc(-c2ccnc(Nc3cccc(C4CCN(C5COC5)CC4)c3)n2)ccc1OC1CCN(C(=O)CO)CC1(F)F]

The title compound (31 mg) was synthesized using 2-(3,3-Difluoro-piperidin-4-yloxy)-5-{2-[3-(1-oxetan-3-yl-piperidin-4-yl)-phenylamino]-pyrimidin-4-yl}-benzonitrile (50 mg), hydroxy-acetic acid (14 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 56% yield, m/z: 605 (M+H). ¹H NMR (DMSO-d6): 9.62 (1H), 8.57 (2H), 8.49 (1H), 7.83 (1H), 7.62 (1H), 7.53 (2H), 7.23 (1H), 6.86 (1H), 5.37 (1H), 5.26 (1H), 4.57 (1H), 4.46 (1H), 3.39 (1H), 3.18 (1H), 2.84 (3H), 2.69 (1H), 2.03 (1H), 1.85 (4H), 1.68 (2H).

### Example 224: 2-[3,3-Difluoro-1-((S)-2-hydroxy-propionyl)-piperidin-4-yloxy]-5-{2-[3-(1-oxetan-3-yl-piperidin-4-yl)-phenylamino]-pyrimidin-4-yl}-benzonitrile

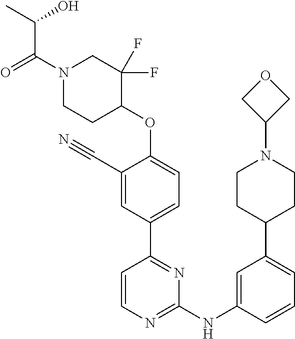 [SMILES: C[C@H](O)C(=O)N1CCC(Oc2ccc(-c3ccnc(Nc4cccc(C5CCN(C6COC6)CC5)c4)n3)cc2C#N)C(F)(F)C1]

The title compound (14.6 mg) was synthesized using 2-(3,3-Difluoro-piperidin-4-yloxy)-5-{2-[3-(1-oxetan-3-yl-piperidin-4-yl)-phenylamino]-pyrimidin-4-yl}-benzonitrile (50 mg), (S)-2-Hydroxy-propionic acid (21 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 25% yield, m/z: 619 (M+H). ¹H NMR (DMSO-d6): 9.62 (1H), 8.57 (2H), 8.49 (1H), 7.83 (1H), 7.62 (1H), 7.53 (2H), 7.23 (1H), 6.86 (1H), 5.37 (1H), 5.26 (1H), 4.57 (1H), 4.46 (1H), 3.39 (1H), 3.18 (1H), 2.84 (3H), 2.69 (1H), 2.03 (1H), 1.85 (4H), 1.68 (2H), 1.22 (3H).

### Example 225: 2-[3,3-Difluoro-1-((R)-2-hydroxy-propionyl)-piperidin-4-yloxy]-5-{2-[3-(1-oxetan-3-yl-piperidin-4-yl)-phenylamino]-pyrimidin-4-yl}-benzonitrile

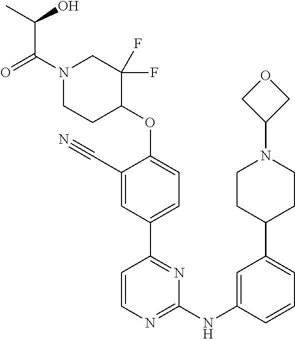 [SMILES: C[C@@H](O)C(=O)N1CCC(Oc2ccc(-c3ccnc(Nc4cccc(C5CCN(C6COC6)CC5)c4)n3)cc2C#N)C(F)(F)C1]

The title compound (18.7 mg) was synthesized using 2-(3,3-Difluoro-piperidin-4-yloxy)-5-{2-[3-(1-oxetan-3-yl-piperidin-4-yl)-phenylamino]-pyrimidin-4-yl}-benzonitrile (50 mg), (R)-2-Hydroxy-propionic acid (21 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 32% yield, m/z: 619 (M+H). ¹H NMR (DMSO-d6): 9.62 (1H), 8.57 (2H), 8.49 (1H), 7.83 (1H), 7.62 (1H), 7.53 (2H), 7.23 (1H), 6.86 (1H), 5.37 (1H), 5.26 (1H), 4.57 (1H), 4.46 (1H), 3.39 (1H), 3.18 (1H), 2.84 (3H), 2.69 (1H), 2.03 (1H), 1.85 (4H), 1.68 (2H), 1.22 (3H).

### Example 226: 2-([33-difluoro-1-[(2R)-2-hydroxypropanoyl]piperidin-4-yl]oxy)-5-[2-([4-[1-(oxetan-3-yl)piperidin-4-yl]phenyl]amino)pyrimidin-4-yl]benzonitrile

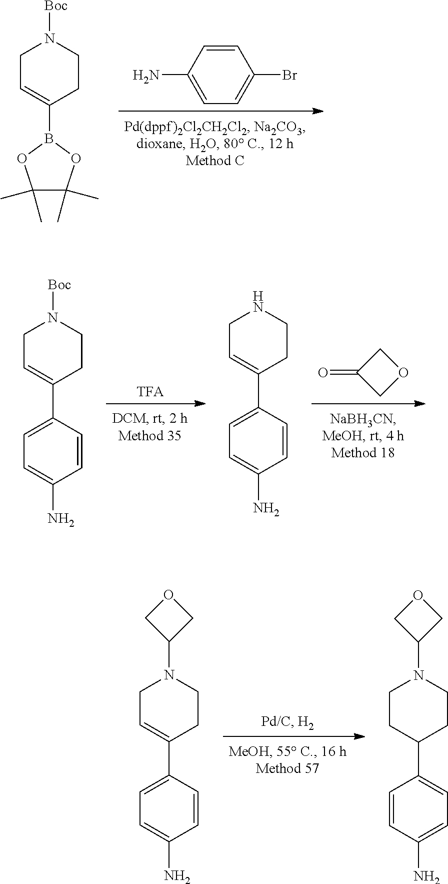

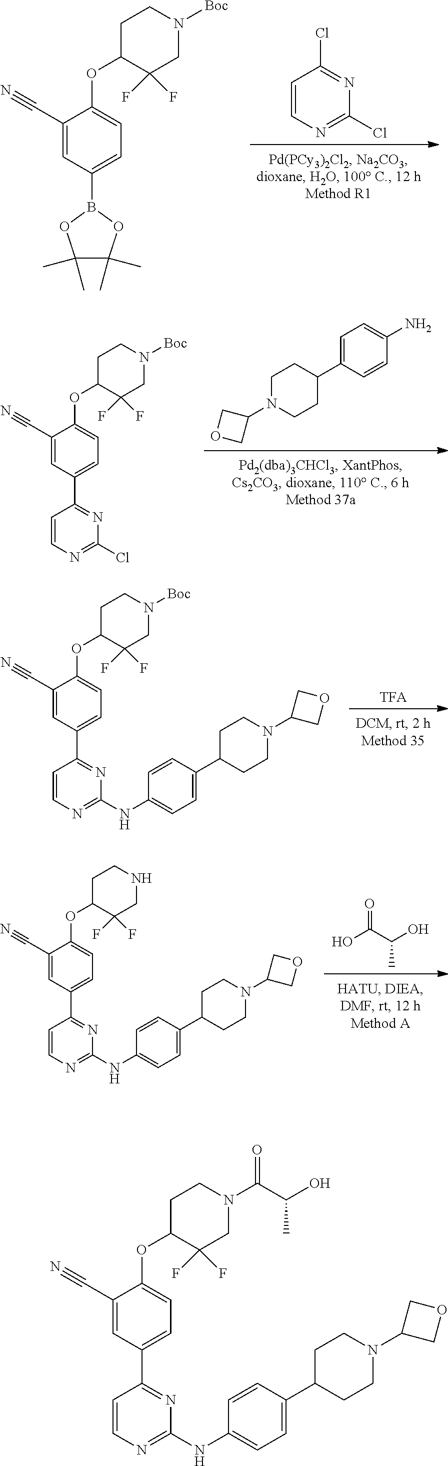

4-(4-nitrophenyl)-1-(oxetan-3-yl)-1,2,3,6-tetrahydropyridine: The title compound was prepared from tert-butyl 4-(4,4,5,5-tetramethyl-1,3,2-dioxaborolan-2-yl)-5,6-dihydropyridine-1 (2H)-carboxylate, 4-bromobenzenamine and oxetan-3-one using Method C, 15 and 18 to yield 4-(4-nitrophenyl)-1-(oxetan-3-yl)-1,2,3,6-tetrahydropyridine as a light yellow solid (1.06 g, 29% for 3 steps). MS: m/z=261.0 [M+H]⁺.

### Method 57

4-[1-(oxetan-3-yl)piperidin-4-yl]aniline: To a solution of 4-(4-nitrophenyl)-1-(oxetan-3-yl)-1,2,3,6-tetrahydropyridine (0.99 g, 3.80 mmol) in MeOH (20 mL) was added palladium carbon (80 mg, 0.75 mmol) under nitrogen atmosphere. The reaction flask was vacuumed and flushed with hydrogen. Then the reaction mixture was hydrogenated for 16 h at 55° C. under hydrogen atmosphere using a hydrogen balloon. When the reaction was done, the reaction mixture was filtered through a celite pad and the filtrate was concentrated under reduced pressure to yield 4-[1-(oxetan-3-yl)piperidin-4-yl]aniline as a light yellow solid (725 mg, 82%). MS: m/z=233.1 [M+H]⁺.

2-[(3,3-difluoropiperidin-4-yl)oxy]-5-[2-([4-[1-(oxetan-3-yl)piperidin-4-yl]phenyl]amino)pyrimidin-4-yl]benzonitrile: The title compound was prepared from 4-[1-(oxetan-3-yl)piperidin-4-yl]aniline, tert-butyl 4-[4-(2-chloropyrimidin-4-yl)-2-cyanophenoxy]-3,3-difluoropiperidine-1-carboxylate and (2R)-2-hydroxypropanoic acid using Method R1, 37a and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 3% to 12% gradient in 7 min; detector, UV 254 nm. 2-[(3,3-difluoropiperidin-4-yl)oxy]-5-[2-([4-[1-(oxetan-3-yl)piperidin-4-yl]phenyl]amino) pyrimidin-4-yl]benzonitrile was obtained as an yellow solid (4 mg, 1.7% for 3 steps). HPLC: 92.3% purity, RT=6.81 min. MS: m/z=547.2 [M+H]⁺. ¹H NMR (300 MHz, DMSO-d₆, ppm) δ 9.72 (s, 1H), 8.65-8.46 (m, 3H), 7.80-7.73 (m, 2H), 7.68-7.59 (m, 1H), 7.52-7.45 (m, 1H), 7.26-7.17 (m, 2H), 5.44 (br s, 1H), 4.83-4.70 (m, 4H), 4.40 (br s, 1H), 3.80-3.70 (m, 3H), 3.65-3.53 (m, 2H), 3.27-3.08 (m, 2H), 3.09-2.64 (m, 3H), 2.44-1.71 (m, 6H).

2-([3,3-difluoro-1-[(2R)-2-hydroxypropanoyl]piperidin-4-yl]oxy)-5-[2-([4-[1-(oxetan-3-yl)piperidin-4-yl]phenyl]amino)pyrimidin-4-yl]benzonitrile: The title compound was prepared from 2-[(3,3-difluoropiperidin-4-yl)oxy]-5-[2-([4-[1-(oxetan-3-yl)piperidin-4-yl]phenyl]amino)pyrimidin-4-yl]benzonitrile and (2R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 35% to 48% gradient in 7 min; detector, UV 254 nm. 2-([3,3-difluoro-1-[(2R)-2-hydroxypropanoyl]piperidin-4-yl]oxy)-5-[2-([4-[1-(oxetan-3-yl)piperidin-4-yl]phenyl]amino)pyrimidin-4-yl]benzonitrile was obtained as off-white solid (13 mg, 13%). HPLC: 97.8% purity, RT=7.67 min. MS: m/z=619.3 [M+H]⁺. ¹H NMR (300 MHz, DMSO-de, ppm) δ 9.62 (s, 1H), 8.59-8.42 (m, 3H), 7.73-7.61 (m, 3H), 7.49-7.41 (m, 1H), 7.22-7.13 (m, 2H), 5.41-5.34 (m, 1H), 5.28-5.19 (m, 1H), 4.58-4.38 (m, 5H), 4.33-3.54 (m, 3H), 3.52-3.34 (m, 2H), 2.83-2.73 (m, 2H), 2.44-2.38 (m, 1H), 2.28-1.93 (m, 2H), 1.91-1.50 (m, 6H), 1.25-1.16 (m, 3H).

### Example 227: 2-([3,3-difluoro-1-[(2R)-2-hydroxypropanoyl]piperidin-4-yl]oxy)-5-[2-([3-methoxy-4-[1-(oxetan-3-yl)piperidin-4-yl]phenyl]amino)pyrimidin-4-yl]benzonitrile

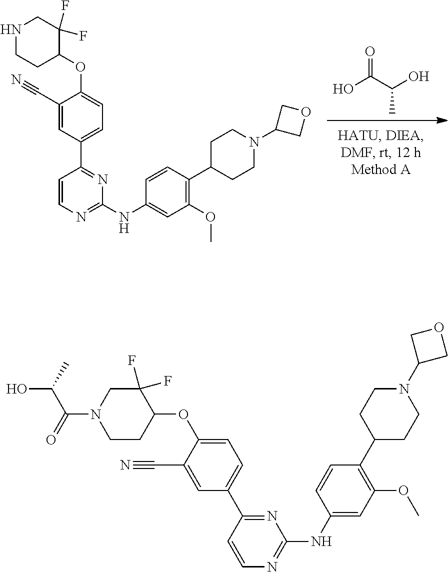

The title compound was prepared from 2-[(3,3-difluoropiperidin-4-yl)oxy]-5-[2-([3-methoxy-4-[1-(oxetan-3-yl)piperidin-4-yl]phenyl]amino)pyrimidin-4-yl]benzonitrile and (2R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 32% to 43% gradient in 8 min; detector, UV 254 nm. 2-([3,3-difluoro-1-[(2R)-2-hydroxypropanoyl]piperidin-4-yl]oxy)-5-[2-([3-methoxy-4-[1-(oxetan-3-yl)piperidin-4-yl]phenyl]amino)pyrimidin-4-yl]benzonitrile was obtained as a light yellow solid (23 mg, 21%). HPLC: 97.9% purity, RT=4.80 min. MS: m/z=649.2 [M+H]⁺. ¹H NMR (300 MHz, DMSO-d₆, ppm) δ 9.66 (s, 1H), 8.71-8.46 (m, 3H), 7.79-7.62 (m, 2H), 7.56-7.45 (m, 1H), 7.33-7.19 (m, 1H), 7.17-7.07 (m, 1H), 5.39 (br s, 1H), 5.28-5.19 (m, 1H), 4.63-4.37 (m, 5H), 4.33-3.80 (m, 2H), 3.83 (s, 3H), 3.73-3.45 (m, 2H), 3.45-3.35 (m, 1H), 2.93-2.69 (m, 3H), 2.25-1.94 (m, 2H), 1.92-1.74 (m, 2H), 1.75-1.55 (m, 4H), 1.26-1.15 (m, 3H).

### Example 228: 2-([33-difluoro-1-[(2S)-2-hydroxypropanoyl]piperidin-4-yl]oxy)-5-[2-([2-methoxy-4-[4-(oxetan-3-yl)piperazin-1-yl]phenyl]amino)pyrimidin-4-yl]benzonitrile

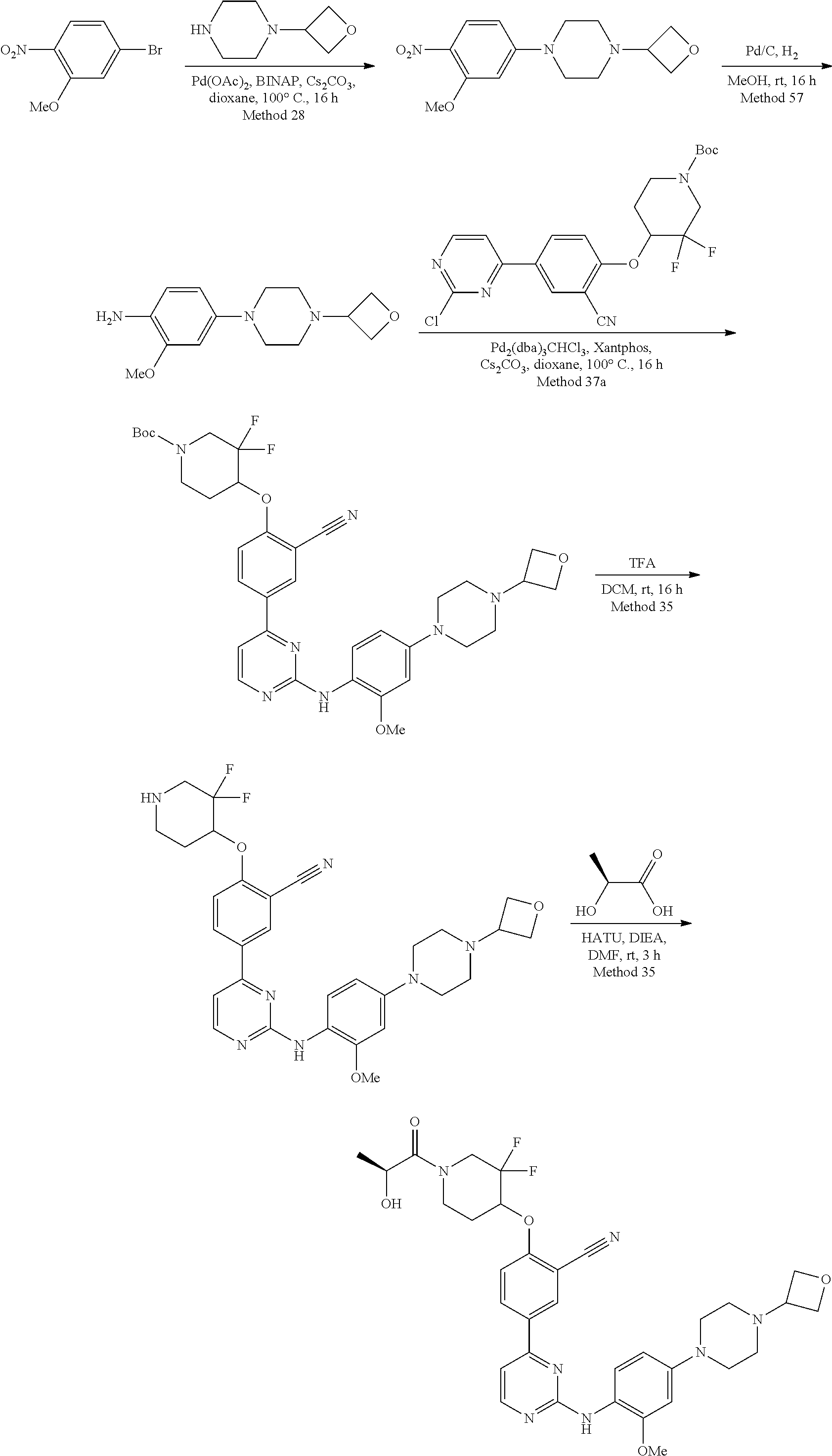

2-[(3,3-difluoropiperidin-4-yl)oxy]-5-[2-([2-methoxy-4-[4-(oxetan-3-yl)piperazin-1-yl]phenyl]amino)pyrimidin-4-yl]benzonitrile: The title compound was prepared from 4-bromo-2-methoxy-1-nitrobenzene, 1-(oxetan-3-yl)piperazine, and 4-[4-(2-chloropyrimidin-4-yl)-2-cyanophenoxy]-3,3-difluoropiperidine-1-carboxylate using Method 28, 57, 37a, and 35. The final product was purified by prep-HPLC under the following condition: column, Atlantis HILIC OBD C18 Column, 150×19 mm, 5 um; mobile phase, acetonitrile in water (with 10 mmol/L NH₄HCO₃ and 0.1% NH₃.H₂O), 25% to 49% gradient in 8 min; detector, UV 254 nm. 2-[(3,3-difluoropiperidin-4-yl)oxy]-5-[2-([2-methoxy-4-[4-(oxetan-3-yl)piperazin-1-yl]phenyl]amino) pyrimidin-4-yl]benzonitrile was obtained as an yellow solid (5 mg, 1.6% for 4 steps). HPLC: 99.5% purity, RT=3.61 min. MS: m/z=578.1 [M+H]⁺. ¹H NMR (300 MHz, DMSO-d₆, ppm) δ 8.52-8.34 (m, 3H), 8.11 (s, 1H), 7.78-7.68 (m, 1H), 7.63-7.54 (m, 1H), 7.41-7.32 (m, 1H), 6.68-6.61 (m, 1H), 6.56-6.45 (m, 1H), 5.21-5.14 (m, 1H), 4.62-4.51 (m, 2H), 4.52-4.41 (m, 2H), 3.80 (s, 3H), 3.49-3.38 (m, 1H), 3.18-3.10 (m, 5H), 3.04-2.57 (m, 4H), 2.45-2.35 (m, 4H), 2.16-1.69 (m, 2H).

2-([3,3-difluoro-1-[(2S)-2-hydroxypropanoyl]piperidin-4-yl]oxy)-5-[2-([2-methoxy-4-[4-(oxetan-3-yl)piperazin-1-yl]phenyl]amino)pyrimidin-4-yl]benzonitrile: The title compound was prepared from 2-[(3,3-difluoropiperidin-4-yl)oxy]-5-[2-([2-methoxy-4-[4-(oxetan-3-yl)piperazin-1-yl]phenyl]amino)pyrimidin-4-yl]benzonitrile and (2S)-2-hydroxypropanoic acid using Method A. The final product was purified by prep-HPLC under the following condition: column, Atlantis HILIC OBD C18 Column, 150×19 mm, 5 um; mobile phase, acetonitrile in water (with 10 mmol/L NH₄HCO₃ and 0.1% NH₃.H₂O), 25% to 49% gradient in 8 min; detector, UV 254 nm. 2-([3,3-difluoro-1-[(2S)-2-hydroxypropanoyl]piperidin-4-yl]oxy)-5-[2-([2-methoxy-4-[4-(oxetan-3-yl)piperazin-1-yl]phenyl]amino)pyrimidin-4-yl]benzonitrile was obtained as a light yellow solid (31 mg, 18%). HPLC: 98.6% purity, RT=4.40 min. MS: m/z=650.2 [M+H]⁺. ¹H NMR (300 MHz, DMSO-d₆, ppm) δ 8.56-8.39 (m, 3H), 8.14 (s, 1H), 7.79-7.70 (m, 1H), 7.69-7.59 (m, 1H), 7.44-7.35 (m, 1H), 6.70-6.62 (m, 1H), 6.57-6.47 (m, 1H), 5.41-5.34 (m, 1H), 5.29-5.20 (m, 1H), 4.64-4.43 (m, 5H), 4.33-3.92 (m, 3H), 3.82 (s, 3H), 3.72-3.40 (m, 2H), 3.22-3.12 (m, 4H), 2.47-2.37 (m, 4H), 2.24-1.73 (m, 2H), 1.22 (d, J=6.5 Hz, 3H).

### Example 229: 2-([3,3-difluoro-1-[(2S)-2-hydroxypropanoyl]piperidin-4-yl]oxy)-5-[2-([3-ethoxy-4-[4-(oxetan-3-yl)piperazin-1-yl]phenyl]amino)pyrimidin-4-yl]benzonitrile

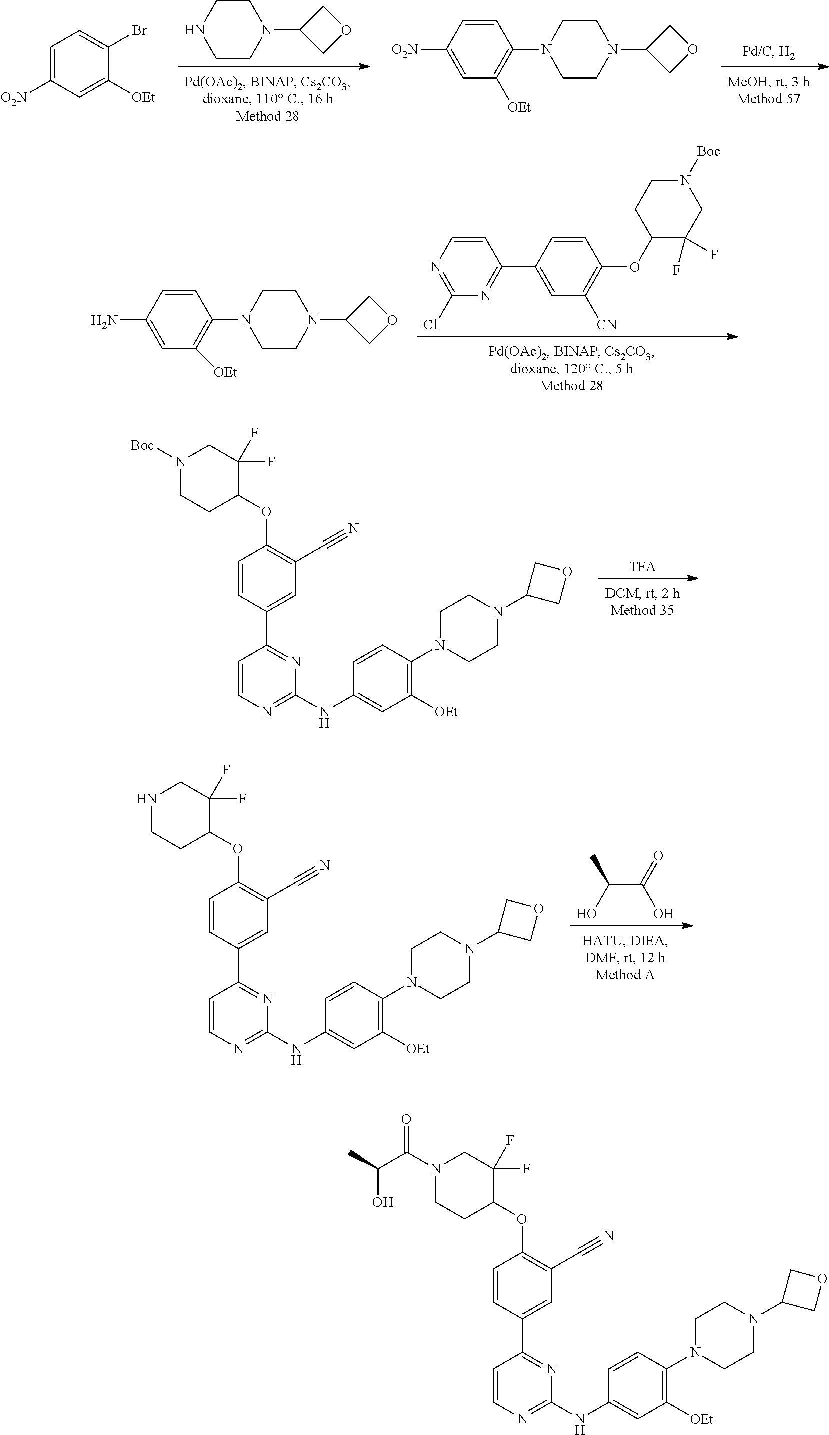

2-[(3,3-difluoropiperidin-4-yl)oxy]-5-[2-([3-ethoxy-4-[4-(oxetan-3-yl)piperazin-1-yl]phenyl]amino)pyrimidin-4-yl]benzonitrile: The title compound was prepared from 1-bromo-2-ethoxy-4-nitrobenzene, 1-(oxetan-3-yl)piperazine, and tert-butyl 4-(4-(2-chloro pyrimidin-4-yl)-2-cyanophenoxy)-3,3-difluoropiperidine-1-carboxylate using Method 28,57, and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 30% to 60% gradient in 8 min; detector, UV 254 nm. 2-[(3,3-difluoropiperidin-4-yl)oxy]-5-[2-[(2-methoxypyridin-4-yl)amino]pyrimidin-4-yl]benzonitrile was obtained as a light yellow solid (5 mg, 2.6% for 4 steps). HPLC: 99.3% purity, RT=2.26 min. MS: m/z=592.0 [M+H]⁺. ¹H NMR (400 MHz, DMSO-d₆, ppm) δ 9.51 (s, 1H), 8.57-8.49 (m, 2H), 8.48-8.40 (m, 1H), 7.66-7.57 (m, 2H), 7.45-7.39 (m, 1H), 7.25-7.17 (m, 1H), 6.87-6.80 (m, 1H), 5.29-5.16 (m, 1H), 4.60-4.52 (m, 2H), 4.51-4.42 (m, 2H), 4.10-4.00 (m, 2H), 3.50-3.42 (m, 1H), 3.22-2.60 (m, 8H), 2.62-2.51 (m, 1H), 2.42-2.38 (m, 4H), 2.14-1.73 (m, 2H), 1.42-1.33 (m, 3H).

2-([3,3-difluoro-1-[(2S)-2-hydroxypropanoyl]piperidin-4-yl]oxy)-5-[2-([3-ethoxy-4-[4-(oxetan-3-yl)piperazin-1-yl]phenyl]amino)pyrimidin-4-yl]benzonitrile: The title compound was prepared from 2-[(3,3-difluoropiperidin-4-yl)oxy]-5-[2-([3-ethoxy-4-[4-(oxetan-3-yl)piperazin-1-yl]phenyl]amino)pyrimidin-4-yl]benzonitrile and (2S)-2-hydroxypropanoic acid using Method A. The final product was purified by prep-HPLC under the following condition: column, Gemini-NX C18 AXAI Packed, 21.2×130 mm Sum; mobile phase, acetonitrile in water (with 10 mmol/L NH₄HCO₃ and 0.1% NH₃.H₂O), 33% to 63% gradient in 8 min; detector, UV 254 nm. 2-([3,3-difluoro-1-[(2S)-2-hydroxypropanoyl]piperidin-4-yl]oxy)-5-[2-([3-ethoxy-4-[4-(oxetan-3-yl)piperazin-1-yl]phenyl]amino)pyrimidin-4-yl]benzonitrile was obtained as a light yellow solid (31 mg, 21%). HPLC: 96.7% purity, RT=3.48 min. MS: m/z=665.1 [M+H]⁺. ¹H NMR (400 MHz, DMSO-d₆, ppm) δ 9.54 (s, 1H), 8.65-8.44 (m, 3H), 7.70-7.58 (m, 2H), 7.47-7.41 (m, 1H), 7.26-7.18 (m, 1H), 6.88-6.80 (m, 1H), 5.39 (s, 1H), 5.29-5.20 (m, 1H), 4.60-4.43 (m, 5H), 4.20-4.15 (m, 1H), 4.11-4.00 (m, 2H), 3.98-3.55 (m, 3H), 3.52-3.41 (m, 1H), 3.01-2.96 (m, 4H), 2.43-2.38 (m, 4H), 2.24-1.81 (m, 2H), 1.39 (t, J=6.9 Hz, 3H), 1.23 (d, J=6.5 Hz, 3H).

### Example 230: 2-((S)-3,3-difluoro-1-((R)-2-hydroxypropanoyl)piperidin-4-yloxy)-5-(2-(2-methoxypyridin-4-ylamino)pyrimidin-4-yl)benzonitrile and Example 231: 2-((R)-3,3-difluoro-1-((R)-2-hydroxypropanoyl)piperidin-4-yloxy)-5-(2-(2-methoxypyridin-4-ylamino)pyrimidin-4-yl)benzonitrile

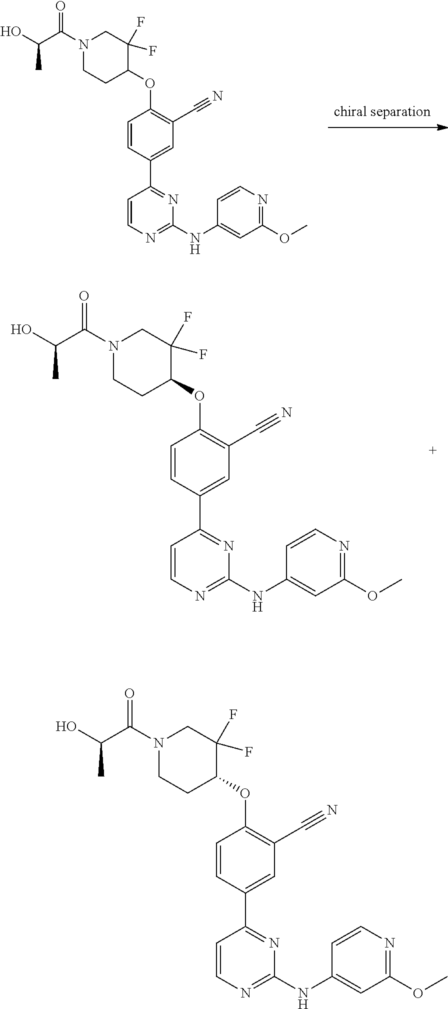 [SMILES: COc1cc(Nc2nccc(-c3ccc(OC4CCN(C(=O)[C@@H](C)O)CC4(F)F)c(C#N)c3)n2)ccn1 ; COc1cc(Nc2nccc(-c3ccc(O[C@H]4CCN(C(=O)[C@@H](C)O)CC4(F)F)c(C#N)c3)n2)ccn1 ; COc1cc(Nc2nccc(-c3ccc(O[C@@H]4CCN(C(=O)[C@@H](C)O)CC4(F)F)c(C#N)c3)n2)ccn1]

The title compounds were obtained by separation on chiral prep-HPLC under the following condition: column, CHIRALPAK IA, 0.46×150 cm, 3 um; mobile phase, MeOH (0.1% DEA), isocratic for 25 min; detector, UV 254 nm.

Example 230: (110 mg, 20%, light yellow solid) HPLC: 98.9% purity, RT=4.38 min. MS: m/z=511.1 [M+H]⁺. ¹H NMR (300 MHz, DMSO-d₆, ppm) δ 10.13 (s, 1H), 8.68-8.60 (m, 1H), 8.60-8.54 (m, 1H), 8.54-8.44 (m, 1H), 8.01-7.92 (m, 1H), 7.74-7.58 (m, 2H), 7.46-7.39 (m, 1H), 7.34-7.25 (m, 1H), 5.40-5.35 (m, 1H), 5.25-5.16 (m, 1H), 4.54-4.43 (m, 1H), 4.36-3.39 (m, 7H), 2.31-1.70 (m, 2H), 1.21 (d, J=6.5 Hz, 3H).

Example 231: (110 mg, 20%, light yellow solid) HPLC: 96.2% purity, RT=4.35 min. MS: m/z=511.1 [M+H]⁺. ¹H NMR (300 MHz, DMSO-d₆, ppm) δ 10.13 (s, 1H), 8.68-8.60 (m, 1H), 8.60-8.54 (m, 1H), 8.54-8.43 (m, 1H), 8.01-7.92 (m, 1H), 7.73-7.58 (m, 2H), 7.46-7.39 (m, 1H), 7.34-7.25 (m, 1H), 5.40-5.35 (m, 1H), 5.26-5.18 (m, 1H), 4.54-4.43 (m, 1H), 4.32-3.38 (m, 7H), 2.25-1.77 (m, 2H), 1.21 (d, J=6.4 Hz, 3H).

### Example 232: 2-(3,3-Difluoro-piperidin-4-yloxy)-5-[2-(2-methoxy-pyrimidin-4-ylamino)-pyrimidin-4-yl]-benzonitrile

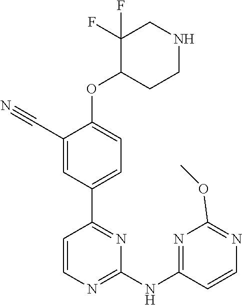 [SMILES: COc1nccc(Nc2nccc(-c3ccc(OC4CCNCC4(F)F)c(C#N)c3)n2)n1]

The title compound (100 mg) was synthesized with 4-{2-Cyano-4-[2-(2-methoxy-pyrimidin-4-ylamino)-pyrimidin-4-yl]-phenoxy}-3,3-difluoro-piperidine-1-carboxylic acid tert-butyl ester (300 mg) and HCl in dioxane (4M) using Method 17 in 41% yield, m/z: 440 (M+H). ¹H NMR (DMSO-d6): 8.62 (2H), 8.54 (1H), 7.77 (2H), 7.66 (1H), 7.50 (1H), 7.37 (2H), 7.00 (2H), 5.46 (1H), 3.74 (5H), 3.24 (2H), 2.38 (1H), 2.20 (1H).

### Example 233: 4-(2-Cyano-4-{2-[6-methoxy-5-(4-oxetan-3-yl-piperazin-1-yl)-pyridin-2-ylamino]-pyrimidin-4-yl}-phenoxy)-4-methy 1-piperidine-1-carboxylic acid tert-butyl ester

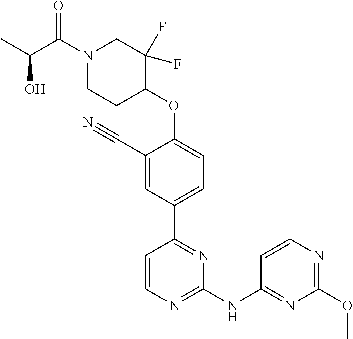 [SMILES: COc1nccc(Nc2nccc(-c3ccc(OC4CCN(C(=O)[C@H](C)O)CC4(F)F)c(C#N)c3)n2)n1]

The title compound (32.2 mg) was synthesized using 2-(3,3-Difluoro-piperidin-4-yloxy)-5-[2-(2-methoxy-pyrimidin-4-ylamino)-pyrimidin-4-yl]-benzonitrile (100 mg) and (R)-2-Hydroxy-propionic acid (40.50 mg) using Method A in 27% yield, m/z: 512 (M+H). ¹H NMR (DMSO-d6): 10.4 (1H), 8.72 (2H), 6.64 (1H), 8.54 (1H), 8.41 (1h), 7.99 (2H), 7.76 (1H), 7.68 (1H), 5.39 (1H), 5.23 (1H), 4.49 (1H), 4.10 (1H), 4.06 (1H), 3.90 93H), 3.65 (1H), 2.18 (1H), 2.00 (1H). 1.23 (3H).

### Example 234: 2-[[3,3-difluoro-1-(2-hydroxyacetyl)piperidin-4-yl]oxy]-5-(2-[[6-methoxy-5-(l-methylpiperidin-4-yl)pyridin-2-yl]amino]pyrimidin-4-yl)benzonitrile

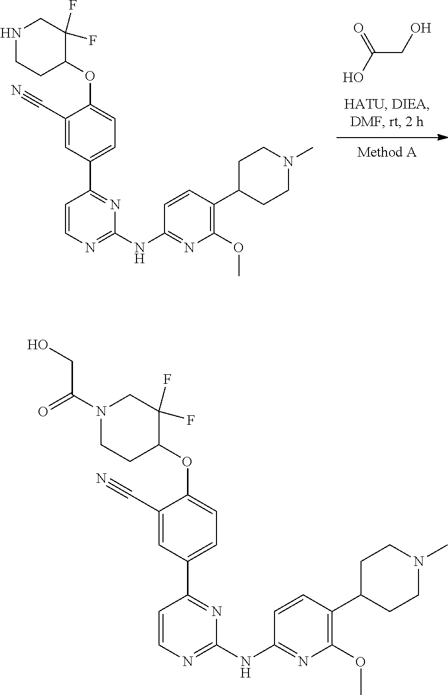 [SMILES: COc1nc(Nc2nccc(-c3ccc(OC4CCNCC4(F)F)c(C#N)c3)n2)ccc1C1CCN(C)CC1 ; O=C(O)CO ; COc1nc(Nc2nccc(-c3ccc(OC4CCN(C(=O)CO)CC4(F)F)c(C#N)c3)n2)ccc1C1CCN(C)CC1]

2-[[3,3-difluoro-1-(2-hydroxyacetyl)piperidin-4-yl]oxy]-5-(2-[[6-methoxy-5-(1-methylpiperidin-4-yl)pyridin-2-yl]amino]pyrimidin-4-yl)benzonitrile 2-[[3,3-difluoro-1-(2-hydroxyacetyl)piperidin-4-yl]oxy]-5-(2-[[6-methoxy-5-(1-methylpiperidin-4-yl)pyridin-2-yl]amino]pyrimidin-4-yl)benzonitrile was prepared from 2-[(3,3-difluoropiperidin-4-yl)oxy]-5-(2-[[6-methoxy-5-(1-methylpiperidin-4-yl)pyridin-2-yl]amino]pyrimidin-4-yl)benzonitrile and 2-hydroxyacetic acid using Method A. The final product was purified by prep-HPLC under the following conditions: column, Atlantis HILIC OBD Column, 150×19 mm, 5 um; mobile phase, acetonitrile in water (with 10 mmol/L NH₄HCO₃ and 0.1% NH₃.H₂O), 32% to 58% gradient in 8 min; detector, UV 254 nm. 2-[[3,3-difluoro-1-(2-hydroxyacetyl)piperidin-4-yl]oxy]-5-(2-[[6-methoxy-5-(1-methylpiperidin-4-yl)pyridin-2-yl]amino]pyrimidin-4-yl)benzonitrile was obtained as off-white solid (16 mg, 20%). HPLC: 93.4% purity, RT=4.37 min. MS: m/z=594.4 [M+H]⁺. 1H NMR (300 MHz, DMSO-d6) δ 9.52 (s, 1H), 8.67-8.50 (m, 3H), 7.84-7.75 (m, 1H), 7.73-7.63 (m, 1H), 7.63-7.51 (m, 2H), 5.43-5.36 (m, 1H), 4.94-4.87 (m, 1H), 4.22-4.16 (m, 2H), 3.89 (s, 3H), 3.86-0.375 (m, 2H), 3.70-3.42 (m, 2H), 2.91-2.81 (m, 2H), 2.68-2.61 (m, 1H), 2.18 (s, 3H), 2.10-1.87 (m, 4H), 1.75-1.53 (m, 4H).

### Example 235: 2-((3S,4R)-3-Fluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

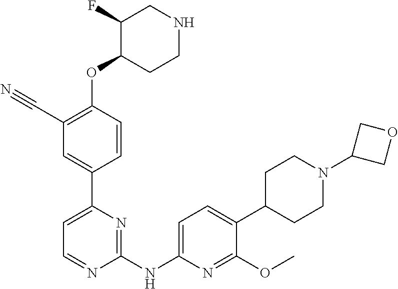 [SMILES: COc1nc(Nc2nccc(-c3ccc(O[C@@H]4CCNC[C@@H]4F)c(C#N)c3)n2)ccc1C1CCN(C2COC2)CC1]

The title compound (25 mg) was synthesized using (3S,4R)-4-{2-Cyano-4-[2-(2-methoxy-1′-oxetan-3-yl-1′,2′,3′,4′,5′,6′-hexahydro-[3,4′]bipyridinyl-6-ylamino)-pyrimidin-4-yl]-phenoxy}-3-fluoro-piperidine-1-carboxylic acid tert-butyl ester (300 mg) with TFA in 10% yield, m/z: 560 (M+H). ¹H NMR (DMSO-d6): 9.42 (1H), 8.62 (2H), 8.52 (1H), 7.81 2H), 7.59 (3H), 5.07 (1H), 5.02 (1H), 4.91 (1H), 4.79 (1H), 4.55 (2H), 4.49 (2H), 3.90 (3H), 3.42 (1H), 3.18 (1H), 2.91 (2H), 2.81 (1H), 2.73 (2H), 1.86 (4H), 1.70 (5H).

### Example 236: 2-[3,3-Difluoro-1-((S)-2-hydroxy-3-methyl-butyryl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

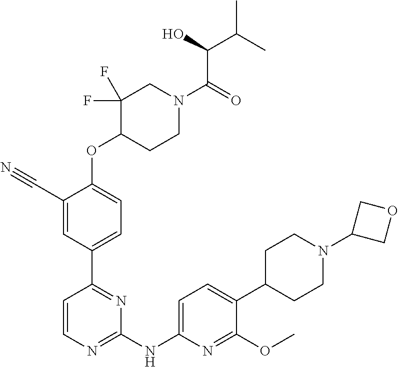 [SMILES: COc1nc(Nc2nccc(-c3ccc(OC4CCN(C(=O)[C@@H](O)C(C)C)CC4(F)F)c(C#N)c3)n2)ccc1C1CCN(C2COC2)CC1]

The title compound (19.5 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), (S)-2-Hydroxy-3-methyl-butyric acid (20 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 31% yield, m/z: 678 (M+H). ¹H NMR (DMSO-d6): 9.50 (1H), 8.62 (2H), 8.56 (1H), 7.81 (1H), 7.65 (1H), 7.59 (2H), 7.43 (1H), 5.42 (1H), 5.37 (1H), 4.56 (2H), 4.45 (2H), 3.90 (3H), 3.42 (1H), 2.81 (2H), 2.73 (2H), 2.14 (1H), 1.92 (1H), 1.84 (2H), 1.72 (4H), 0.88 (6H).

### Example 237: 2-[3,3-Difluoro-1-(1-hydroxy-cyclopropanecarbonyl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

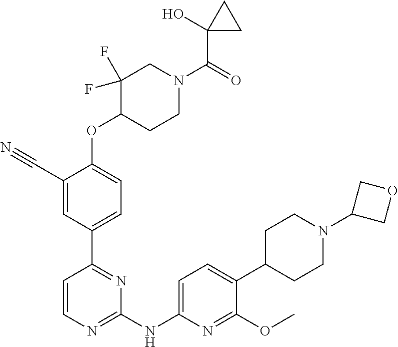 [SMILES: COc1nc(Nc2nccc(-c3ccc(OC4CCN(C(=O)C5(O)CC5)CC4(F)F)c(C#N)c3)n2)ccc1C1CCN(C2COC2)CC1]

The title compound (28.8 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), 1-Hydroxy-cyclopropanecarboxylic acid (17 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 50% yield, m/z: 662 (M+H). ¹H NMR (DMSO-d6): 9.50 (1H), 8.62 (2H), 8.56 (1H), 7.81 (1H), 7.65 (1H), 7.59 (2H), 6.52 (1H), 5.37 (1H), 4.56 (2H), 4.45 (2H), 3.90 (3H), 3.42 (1H), 2.81 (2H), 2.73 (2H), 2.14 (1H), 1.92 (1H), 1.84 (2H), 1.72 (4H), 1.01 (2H), 0.88 (2H).

### Example 238: 2-[3,3-Difluoro-1-((R)-2-hydroxy-3-methyl-butyryl)-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

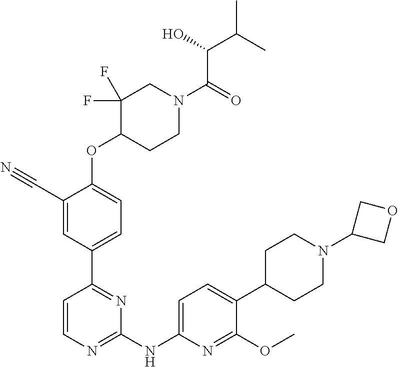 [SMILES: COc1nc(Nc2nccc(-c3ccc(OC4CCN(C(=O)[C@H](O)C(C)C)CC4(F)F)c(C#N)c3)n2)ccc1C1CCN(C2COC2)CC1]

The title compound (24.7 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), (R)-2-Hydroxy-3-methyl-butyric acid (20 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 42% yield, m/z: 678 (M+H). ¹H NMR (DMSO-d6): 9.47 (1H), 8.62 (2H), 8.52 (1H), 7.81 (1H), 7.65 (1H), 7.59 (1H), 5.63 (1H0, 5.38 (1H), 4.56 (2H), 4.45 (2H), 3.90 (3H), 3.42 (1H), 2.81 (2H), 2.68 (1H), 2.13 (1H), 1.96 (1H), 1.86 (2H), 1.63 (4H), 0.87 (6H).

### Example 239: 2-[1-(2-Cyclopropyl-2-hydroxy-acetyl)-3,3-difluoro-piperidin-4-yloxy]-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile

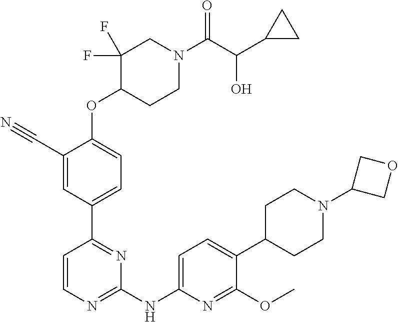 [SMILES: COc1nc(Nc2nccc(-c3ccc(OC4CCN(C(=O)C(O)C5CC5)CC4(F)F)c(C#N)c3)n2)ccc1C1CCN(C2COC2)CC1]

The title compound (19 mg) was synthesized using 2-(3,3-Difluoro-piperidin-4-yloxy)-5-[2-(2-methoxy-1′-oxetan-3-yl-1′,2′,3′,4′,5′,6′-hexahydro-[3,4′]bipyridinyl-6-ylamino)-pyrimidin-4-yl]-benzonitrile (50 mg), Cyclopropyl-hydroxy-acetic acid (21 mg), O-(7-Azabenzotriazol-1-yl)-N,N,N′,N′-tetramethyluronium hexaflurophosphate (HATU) (57 mg) and Ethyl-diisopropyl-amine (55 mg) using Method A in 31% yield, m/z: 676 (M+H). ¹H NMR (DMSO-d6): 9.47 (1H), 8.62 (2H), 8.52 (1H), 7.81 (1H), 7.65 (1H), 7.59 (1H), 5.63 (1H0, 5.38 (1H), 4.56 (2H), 4.45 (2H), 3.90 (3H), 3.42 (1H), 2.81 (2H), 2.68 (1H), 2.13 (1H), 1.96 (1H), 1.86 (2H), 1.63 (4H), 0.45 (4H), 0.31 (1H).

### Example 240: 2-[[(4S)-3,3-difluoro-1-[(2S)-2-hydroxypropanoyl]piperidin-4-yl]oxy]-5-[2-[(6-methoxypyridin-2-yl)amino]pyrimidin-4-yl]benzonitrile and Example 241: 2-[[(4R)-3,3-difluoro-1-[(2S)-2-hydroxypropanoyl]piperidin-4-yl]oxy]-5-[2-[(6-methoxypyridin-2-yl)amino]pyrimidin-4-yl]benzonitrile

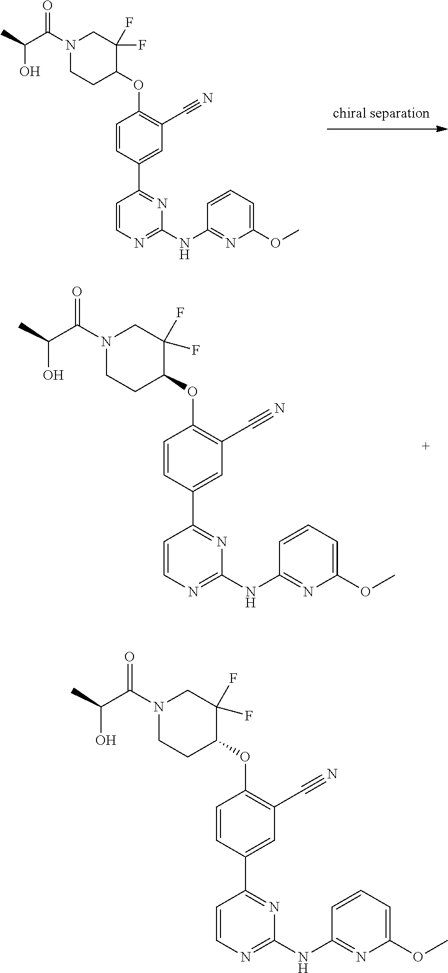

The title compounds were obtained by separation on chiral prep-HPLC under the following condition: column, Lux 3 um Cellulose-4, 4.6×100 cm, 3 um; mobile phase, EtOH: MeCN=1:1 (10 mM NH₃), isocratic for 15 min; detector, UV 254 nm.

2-[[(4S)-3,3-difluoro-1-[(2S)-2-hydroxypropanoyl]piperidin-4-yl]oxy]-5-[2-[(6-methoxypyridin-2-yl)amino]pyrimidin-4-yl]benzonitrile: (70 mg, 19%, white solid) HPLC: 99.2% purity, RT=5.28 min. MS: m/z=511.0 [M+H]⁺. ¹H NMR (300 MHz, DMSO-d₆, ppm) δ 9.62 (s, 1H), 8.71-8.60 (m, 2H), 8.59-8.47 (m, 1H), 7.90-7.78 (m, 1H), 7.74-7.56 (m, 3H), 6.47-6.34 (m, 1H), 5.49-5.29 (m, 1H), 4.56-4.41 (m, 1H), 4.32-3.27 (m, 7H), 2.29-1.71 (m, 2H), 1.21 (d, J=6.5 Hz, 3H).

2-[[(4R)-3,3-difluoro-1-[(2S)-2-hydroxypropanoyl]piperidin-4-yl]oxy]-5-[2-[(6-methoxypyridin-2-yl)amino]pyrimidin-4-yl]benzonitrile: (70 mg, 19%, white solid) HPLC: 99.3% purity, RT=5.28 min. MS: m/z=511.2 [M+H]⁺. ¹H NMR (400 MHz, DMSO-d₆, ppm) δ 9.62 (s, 1H), 8.67-8.59 (m, 2H), 8.58-8.48 (m, 1H), 7.89-7.80 (m, 1H), 7.73-7.63 (m, 2H), 7.63-7.57 (m, 1H), 6.46-6.37 (m, 1H), 5.41-5.35 (m, 1H), 4.51-4.45 (m, 1H), 4.32-3.27 (m, 7H), 2.27-1.71 (m, 2H), 1.21 (d, J=6.4 Hz, 3H).

### Example 242: 2-[1-((S)-2,3-Dihydroxy-propionyl)-3,3-difluoro-piperidin-4-yloxy]-5-[2-(6-methoxy-pyridin-2-ylamino)-pyrimidin-4-yl]-benzonitrile

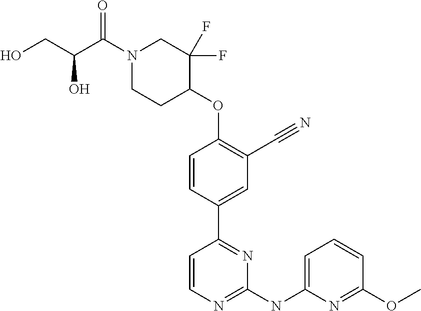 [SMILES: COc1cccc(Nc2nccc(-c3ccc(OC4CCN(C(=O)[C@@H](O)CO)CC4(F)F)c(C#N)c3)n2)n1]

The title compound (12.9 mg) was synthesized using 2-(3,3-Difluoro-piperidin-4-yloxy)-5-[2-(6-methoxy-pyridin-2-ylamino)-pyrimidin-4-yl]-benzonitrile (100 mg) and (S)-2,3-Dihydroxy-propionic acid (48.40 mg) using Method A in 10% yield, m/z: 527 (M+H). ¹H NMR (DMSO-d6): 9.74 (1H), 8.54 (2H), 8.41 (1h), 7.68 (1H), 7.61 (1H), 7.49 (1H), 7.29 (1H), 7.23 (1H), 5.56 (1H), 5.39 (1H), 5.23 (1H), 4.77 (1H), 4.49 (1H), 3.90 (3H), 3.55 (1H), 3.50 (2H).

### Example 243: 2-[1-((S)-2,3-Dihydroxy-propionyl)-3,3-difluoro-piperidin-4-yloxy]-5-[2-(pyridin-2-ylamino)-pyrimidin-4-yl]-benzonitrile

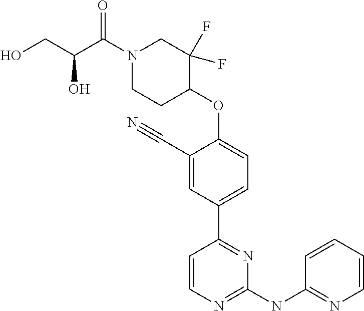 [SMILES: N#Cc1cc(-c2ccnc(Nc3ccccn3)n2)ccc1OC1CCN(C(=O)[C@@H](O)CO)CC1(F)F]

The title compound (17 mg) was synthesized using 2-(3,3-Difluoro-piperidin-4-yloxy)-5-[2-(pyridin-2-ylamino)-pyrimidin-4-yl]-benzonitrile hydrochloride (100 mg) and (S)-2,3-Dihydroxy-propionic acid (48.40 mg) using Method A in 15% yield, m/z: 697 (M+H). ¹H NMR (DMSO-d6): 10.4 (1H), 8.72 (2H), 6.64 (1H), 8.54 (1H), 8.41 (1H), 7.99 (2H), 7.76 (1H), 7.68 (1H), 5.39 (1H), 5.23 (1H), 4.49 (1H), 4.10 (1H), 4.06 (1H), 3.90 93H), 3.65 (1H), 2.18 (1H), 2.00 (1H). 1.23 (3H).

### Example 244: 2-[1-((S)-2,3-Dihydroxy-propionyl)-3,3-difluoro-piperidin-4-yloxy]-5-[2-(5,6-dimethoxy-pyridin-2-ylamino)-pyrimidin-4-yl]-benzonitrile

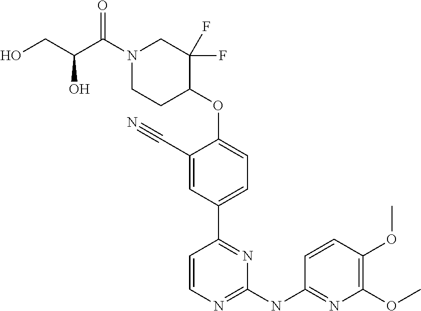 [SMILES: COc1ccc(Nc2nccc(-c3ccc(OC4CCN(C(=O)[C@@H](O)CO)CC4(F)F)c(C#N)c3)n2)nc1OC]

The title compound (35.1 mg) was synthesized using 2-[1-((S)-2,3-Dihydroxy-propionyl)-3,3-difluoro-piperidin-4-yloxy]-5-[2-(5,6-dimethoxy-pyridin-2-ylamino)-pyrimidin-4-yl]-benzonitrile and (S)-2,3-Dihydroxy-propionic acid (48.40 mg) using Method A in 32% yield, m/z: 557 (M+H). ¹H NMR (DMSO-d6): 9.46 (1H), 8.62 (2H), 8.54 (1H), 7.72 (1H), 7.66 (1H), 7.35 (1H), 7.37 (1H), 5.39 (1H), 5.23 (1H), 4.74 (1H), 4.40 (1H), 3.90 (3H), 3.75 (3H), 3.57 (1H), 3.53 91H).

### Example 245: 2-([33-difluoro-1-[(2S)-2-hydroxypropanoyl]piperidin-4-yl]oxy)-5-[2-([5-[4-(oxetan-3-yl)piperazin-1-yl]pyridin-2-yl]amino)pyrimidin-4-yl]benzonitrile

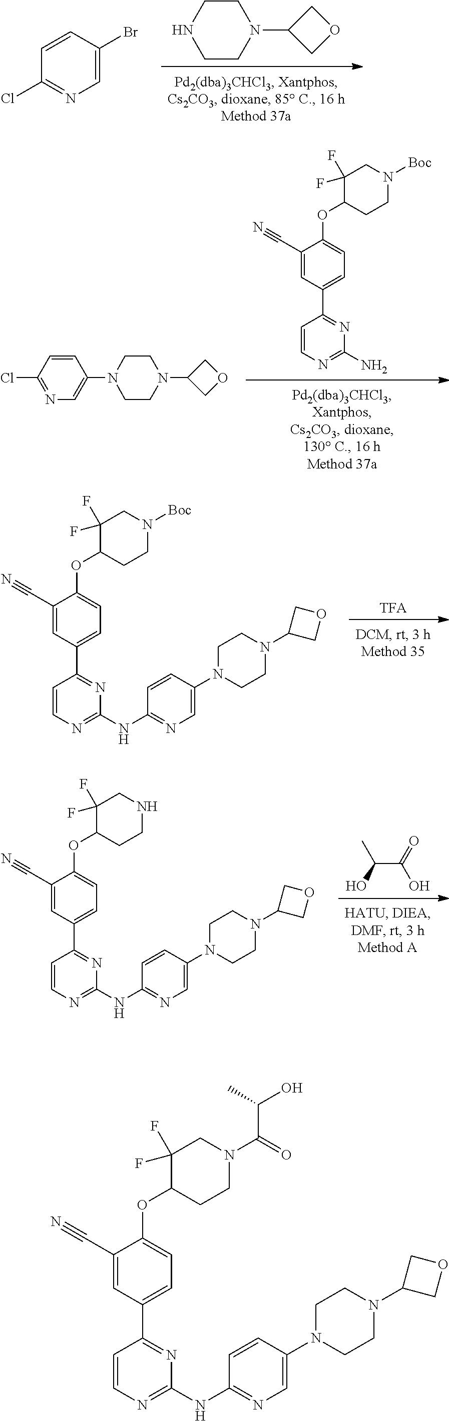

The title compound was prepared from 5-bromo-2-chloropyridine, 1-(oxetan-3-yl)piperazine, tert-butyl 4-(4-(2-aminopyrimidin-4-yl)-2-cyanophenoxy)-3,3-difluoropiperidine-1-carboxylate and (S)-2-hydroxypropanoic acid using Method 37a, 37a, 35 and A. The final product was purified by prep-HPLC under the following conditions: column, XBridge Prep OBD C18 Column, 150×19 mm, 5 um; mobile phase, EtOH in water (with 10 mmol/L NH₄HCO₃ and 0.1% NH₃.H₂O), 43% to 65% gradient in 8 min; detector, UV 254 nm. 2-([3,3-difluoro-1-[(2S)-2-hydroxypropanoyl]piperidin-4-yl]oxy)-5-[2-([5-[4-(oxetan-3-yl)piperazin-1-yl]pyridin-2-yl]amino)pyrimidin-4-yl]benzonitrile was obtained as an yellow solid (24 mg, 7% for 4 steps). HPLC: 93.3% purity, RT=3.61 min. MS: m/z=621.2 [M+H]⁺. ¹H NMR (300 MHz, DMSO-d6, ppm) δ9.65 (s, 1H), 8.60-8.53 (m, 2H), 8.53-8.44 (m, 1H), 8.14-8.05 (m, 1H), 8.05-7.99 (m, 1H), 7.70-7.61 (m, 1H), 7.55-7.41 (m, 2H), 5.41-5.35 (m, 1H), 5.25-5.19 (m, 1H), 4.67-4.41 (m, 5H), 4.29-3.41 (m, 5H), 3.24-3.06 (m, 4H), 2.59-2.50 (m, 4H), 2.24-1.79 (m, 2H), 1.21 (d, J=6.5 Hz, 3H).

### Example 246: 2-[3,3-Difluoro-1-((S)-2-methoxy-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

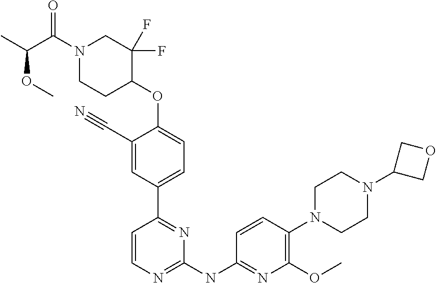 [SMILES: COc1nc(Nc2nccc(-c3ccc(OC4CCN(C(=O)[C@H](C)OC)CC4(F)F)c(C#N)c3)n2)ccc1N1CCN(C2COC2)CC1]

The title compound (10.60 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-2-Methoxy-propionic acid (28.73 mg) using Method A in 11% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.78 (1H), 4.59 (2H), 4.49 (1H), 3.92 (3H), 3.75 (2H), 3.66 (1H), 3.46 (1H), 3.04 (3H), 2.38 (3H), 2.06 (3H), 1.38 (2H).

### Example 247: 2 2-[(S)-3,3-Difluoro-1-((S)-2-hydroxy-propionyl)-piperidin-4-yloxy]-5-{2-[6-methoxy-5-((S)-3-methyl-4-oxetan-3-yl-piperazin-1-yl)-pyridin-2-ylamino]-pyrimidin-4-yl}-benzonitrile

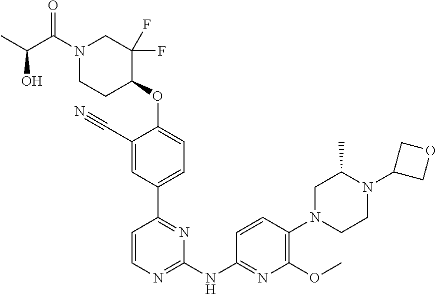 [SMILES: COc1nc(Nc2nccc(-c3ccc(O[C@H]4CCN(C(=O)[C@H](C)O)CC4(F)F)c(C#N)c3)n2)ccc1N1CCN(C2COC2)[C@@H](C)C1]

The title compound (57.70 mg) was separated from 2-[3,3-Difluoro-1-((S)-2-hydroxy-propionyl)-piperidin-4-yloxy]-5-{2-[6-methoxy-5-((S)-3-methyl-4-oxetan-3-yl-piperazin-1-yl)-pyridin-2-ylamino]-pyrimidin-4-yl}-benzonitrile (260 mg) in the SGF chiral column with MeOH containing ammonium hydroxide (20 mM) in 21.5% yield. M/Z: 645 (M+H). ¹H NMR (DMSO-d6): H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 248: 2 2-[(R)-3,3-Difluoro-1-((S)-2-hydroxy-propionyl)-piperidin-4-yloxy]-5-{2-[6-methoxy-5-((S)-3-methyl-4-oxetan-3-yl-piperazin-1-yl)-pyridin-2-ylamino]-pyrimidin-4-yl}-benzonitrile

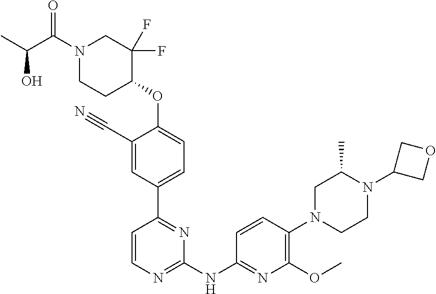 [SMILES: COc1nc(Nc2nccc(-c3ccc(O[C@@H]4CCN(C(=O)[C@H](C)O)CC4(F)F)c(C#N)c3)n2)ccc1N1CCN(C2COC2)[C@@H](C)C1]

The title compound (58.50 mg) was separated from 2-[3,3-Difluoro-1-((S)-2-hydroxy-propionyl)-piperidin-4-yloxy]-5-{2-[6-methoxy-5-((S)-3-methyl-4-oxetan-3-yl-piperazin-1-yl)-pyridin-2-ylamino]-pyrimidin-4-yl}-benzonitrile (260 mg) in the SGF chiral column with MeOH containing ammonium hydroxide (20 mM) in 21.5% yield. M/Z: 645 (M+H). ¹H NMR (DMSO-d6): H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (3H), 0.82 (3H).

### Example 249: 2-([33-difluoro-1-[(2S)-2-hydroxypropanoyl]piperidin-4-yl]oxy)-5-[2-([6-ethoxy-5-[4-(oxetan-3-yl)piperazin-1-yl]pyridin-2-yl]amino)pyrimidin-4-yl]benzonitrile

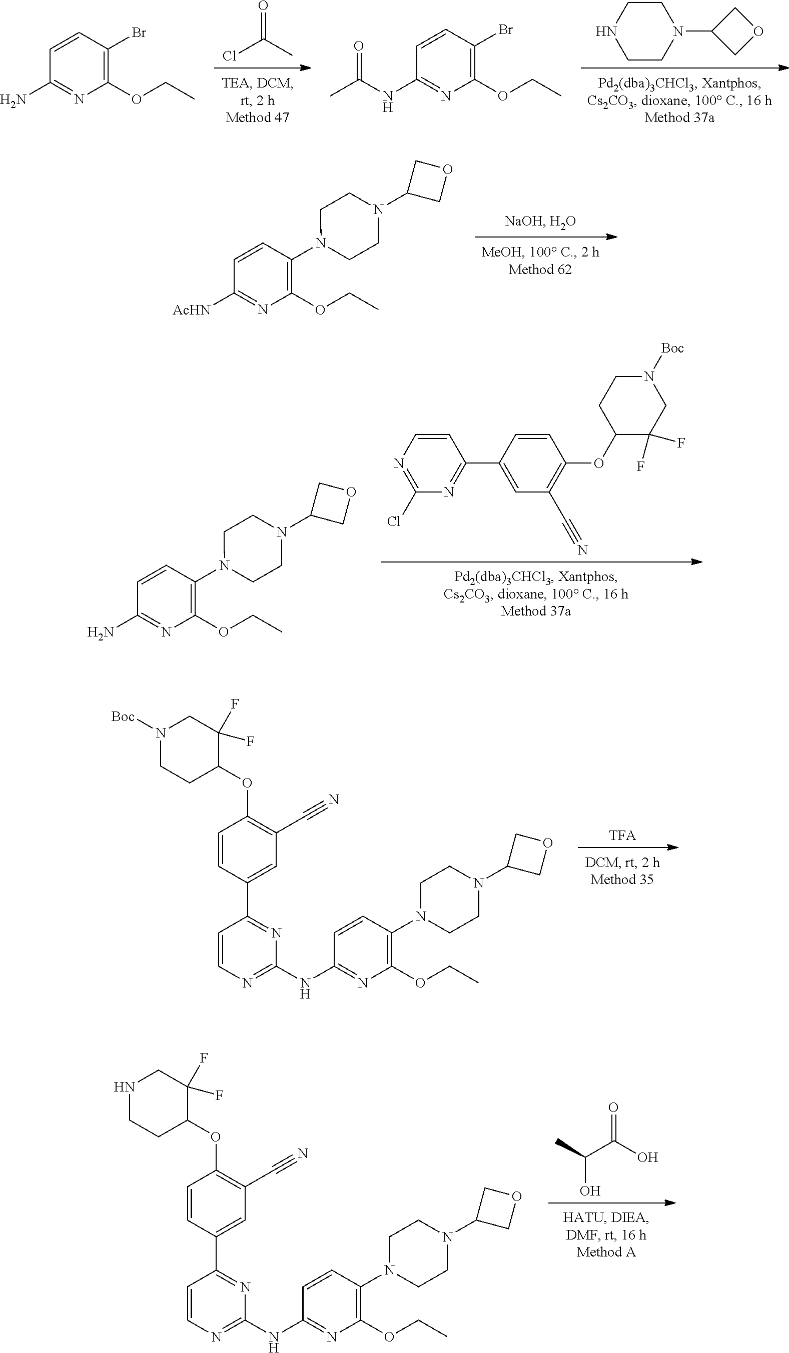

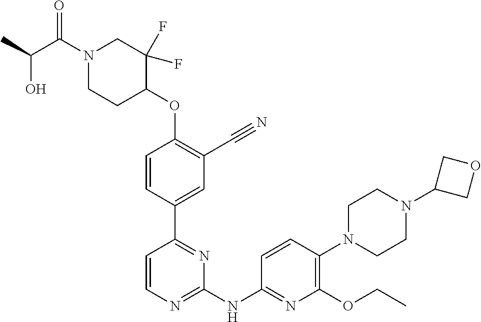 [SMILES: CCOc1nc(Nc2nccc(-c3ccc(OC4CCN(C(=O)[C@H](C)O)CC4(F)F)c(C#N)c3)n2)ccc1N1CCN(C2COC2)CC1]

N-[6-ethoxy-5-[4-(oxetan-3-yl)piperazin-1-yl]pyridin-2-yl]acetamide: N-[6-ethoxy-5-[4-(oxetan-3-yl)piperazin-1-yl]pyridin-2-yl]acetamide was prepared from 5-bromo-6-ethoxypyridin-2-amine, acetyl chloride and 1-(oxetan-3-yl)piperazine using Method 47 and 37a. The final product was purified by flash chromatography eluting with EtOAc in hexane (0% to 90% gradient) to yield N-[6-ethoxy-5-[4-(oxetan-3-yl)piperazin-1-yl]pyridin-2-yl]acetamide as a brown solid (120 mg, 20% for 2 steps). MS: m/z=321.2 [M+H]⁺.

### Method 62

6-ethoxy-5-[4-(oxetan-3-yl)piperazin-1-yl]pyridin-2-amine: To a solution of N-[6-ethoxy-5-[4-(oxetan-3-yl)piperazin-1-yl]pyridin-2-yl]acetamide (106 mg, 0.33 mmol) in MeOH (5 mL) was added sodium hydroxide aqueous solution (3 M, 8 mL, 24 mmol) at room temperature. The resulting mixture was stirred at 100° C. for 3 h. When the reaction was done, the reaction mixture was diluted with H₂O (10 mL) and the resulting mixture was extracted with dichloromethane (30 mL×3). The organic phases were combined, washed with brine and dried over Na₂SO₄. The solvent was removed under reduced pressure and the residue was purified by flash chromatography eluting with EtOAc in hexane (0% to 90% gradient) to yield 6-ethoxy-5-[4-(oxetan-3-yl)piperazin-1-yl]pyridin-2-amine as brown solid (61 mg, 66%). MS: m/z=279.1 [M+H]⁺.

2-[(3,3-difluoropiperidin-4-yl)oxy]-5-[2-([6-ethoxy-5-[4-(oxetan-3-yl)piperazin-1-yl]pyridin-2-yl]amino)pyrimidin-4-yl]benzonitrile: The title compound was prepared from 6-ethoxy-5-(4-(oxetan-3-yl)piperazin-1-yl)pyridin-2-amine and tert-butyl 4-(4-(2-chloropyrimidin-4-yl)-2-cyanophenoxy)-3,3-difluoropiperidine-1-carboxylate using Method 37a and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 20% to 39% gradient in 8 min; detector, UV 254 nm. 2-[(3,3-difluoropiperidin-4-yl)oxy]-5-[2-([6-ethoxy-5-[4-(oxetan-3-yl)piperazin-1-yl]pyridin-2-yl]amino)pyrimidin-4-yl]benzonitrile was obtained as an yellow solid (40 mg, 22% for 2 steps). HPLC: 99.8% purity, RT=3.66 min. MS: m/z=593.2[M+H]⁺. ¹H NMR (300 MHz, DMSO-d₆, ppm) δ 9.33 (s, 1H), 8.60-8.52 (m, 2H), 8.53-8.42 (m, 1H), 7.74-7.57 (m, 2H), 7.55-7.46 (m, 1H), 7.28-7.19 (m, 1H), 5.28-5.06 (m, 1H), 4.60-4.49 (m, 2H), 4.50-4.39 (m, 2H), 4.41-4.27 (m, 2H), 3.53-3.40 (m, 1H), 3.19-3.09 (m, 1H), 3.04-2.76 (m, 6H), 2.74-2.67 (m, 1H), 2.43-2.36 (m, 4H), 2.12-1.71 (m, 2H), 1.38-1.26 (m, 3H).

2-([3,3-difluoro-1-[(2S)-2-hydroxypropanoyl]piperidin-4-yl]oxy)-5-[2-([6-ethoxy-5-[4-(oxetan-3-yl)piperazin-1-yl]pyridin-2-yl]amino)pyrimidin-4-yl]benzonitrile: The title compound was prepared from 2-[(3,3-difluoropiperidin-4-yl)oxy]-5-[2-([6-ethoxy-5-[4-(oxetan-3-yl)piperazin-1-yl]pyridin-2-yl]amino)pyrimidin-4-yl]benzonitrile and (S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 35% to 50% gradient in 8 min; detector, UV 254 nm. 2-([3,3-difluoro-1-[(2S)-2-hydroxypropanoyl]piperidin-4-yl]oxy)-5-[2-([6-ethoxy-5-[4-(oxetan-3-yl)piperazin-1-yl]pyridin-2-yl]amino)pyrimidin-4-yl]benzonitrile was obtained as an yellow solid (7 mg, 21%). HPLC: 96.7% purity, RT=4.47 min. MS: m/z=665.2[M+H]⁺. ¹H NMR (300 MHz, DMSO-d₆, ppm) δ 9.34 (s, 1H), 8.62-8.46 (m, 3H), 7.74-7.60 (m, 2H), 7.56-7.47 (m, 1H), 7.28-7.19 (m, 1H), 5.40-5.33 (m, 1H), 5.25-5.19 (m, 1H), 4.60-4.41 (m, 5H), 4.41-4.27 (m, 2H), 4.19-3.63 (m, 2H), 3.49-3.42 (m, 2H), 3.01-2.95 (m, 4H), 2.44-2.25 (m, 4H), 2.23-1.67 (m, 2H), 1.34 (t, J=6.6 Hz, 3H), 1.25-1.16 (m, 3H).

### Example 250: 2-[3,3-Difluoro-1-(oxetane-2-carbonyl)-piperidin-4-yloxy]-5-{2-[6-methoxy-5-(4-oxetan-3-yl-piperazin-1-yl)-pyridin-2-ylamino]-pyrimidin-4-yl}-benzonitrile

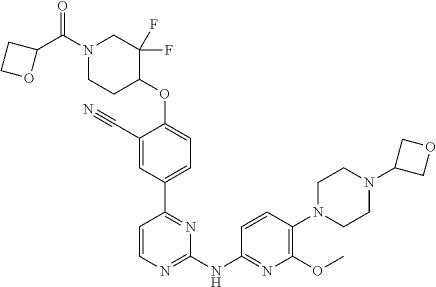 [SMILES: COc1nc(Nc2nccc(-c3ccc(OC4CCN(C(=O)C5CCO5)CC4(F)F)c(C#N)c3)n2)ccc1N1CCN(C2COC2)CC1]

The title compound (12.30 mg) was synthesized using 2-(3,3-Difluoro-piperidin-4-yloxy)-5-[2-(pyridin-2-ylamino)-pyrimidin-4-yl]-benzonitrile hydrochloride (80 mg) and Oxetane-2-carboxylic acid (28.23 mg) using Method A in 13% yield, m/z: 663 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.78 (1H), 4.59 (2H), 4.49 (1H), 3.92 (3H), 3.75 (2H), 3.66 (1H), 3.46 (1H), 3.04 (3H), 2.38 (3H), 2.06 (3H), 1.38 (2H).

### Example 251: 2-[1-(2-Cyano-2-methyl-acetyl)-33-difluoro-piperidin-4-yloxy]-5-{2-[6-methoxy-5-(4-oxetan-3-yl-piperazin-1-yl)-pyridin-2-ylamino]-pyrimidin-4-yl}-benzonitrile

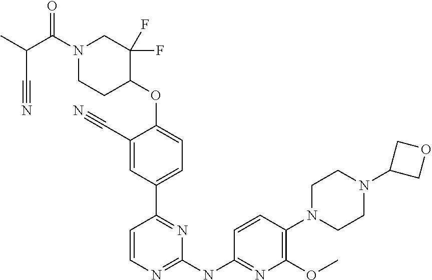 [SMILES: COc1nc(Nc2nccc(-c3ccc(OC4CCN(C(=O)C(C)C#N)CC4(F)F)c(C#N)c3)n2)ccc1N1CCN(C2COC2)CC1]

The title compound (15.10 mg) was synthesized using 2-(3,3-Difluoro-piperidin-4-yloxy)-5-[2-(pyridin-2-ylamino)-pyrimidin-4-yl]-benzonitrile hydrochloride (80 mg) and Cyano-methyl-acetic acid (27.40 mg) using Method A in 16% yield, m/z: 660 (M+H). ¹H NMR (DMSO-d6): 9.49 (1H), 8.61 (2H), 8.51 (1H), 7.78 (1H), 7.65 (1H), 7.56 (1H), 7.39 (1H), 5.44 (1H), 4.59 (2H), 4.49 (1H), 4.25 (1H), 3.92 (3H), 3.55 (2H), 3.47 (2H), 3.04 (3H), 2.38 (3H), 2.23 (1H), 2.09 (1H), 1.30 (3H).

### Example 252: 2-[3,3-Difluoro-1-((S)-3-hydroxy-2-methyl-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

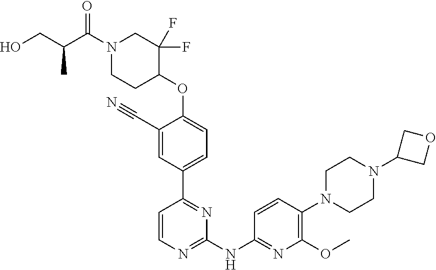 [SMILES: COc1nc(Nc2nccc(-c3ccc(OC4CCN(C(=O)[C@@H](C)CO)CC4(F)F)c(C#N)c3)n2)ccc1N1CCN(C2COC2)CC1]

The title compound (18.2 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-3-Hydroxy-2-methyl-propionic acid (28.70 mg) using Method A in 20% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.26 (2H), 0.98 (3H).

### Example 253: 2-[1-(2-Cyano-acetyl)-3,3-difluoro-piperidin-4-yloxy]-5-{2-[6-methoxy-5-(4-oxetan-3-yl-piperazin-1-yl)-pyridin-2-ylamino]-pyrimidin-4-yl}-benzonitrile

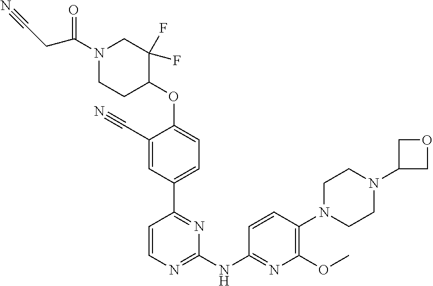 [SMILES: COc1nc(Nc2nccc(-c3ccc(OC4CCN(C(=O)CC#N)CC4(F)F)c(C#N)c3)n2)ccc1N1CCN(C2COC2)CC1]

The title compound (21.10 mg) was synthesized using 2-(3,3-Difluoro-piperidin-4-yloxy)-5-[2-(pyridin-2-ylamino)-pyrimidin-4-yl]-benzonitrile hydrochloride (80 mg) and Cyano-acetic acid (23.70 mg) using Method A in 23% yield, m/z: 646 (M+H). ¹H NMR (DMSO-d6): 9.49 (1H), 8.61 (2H), 8.51 (1H), 7.78 (1H), 7.65 (1H), 7.56 (1H), 7.39 (1H), 5.44 (1H), 4.59 (2H), 4.49 (1H), 4.25 (1H), 3.92 (3H), 3.55 (2H), 3.47 (2H), 3.04 (3H), 2.38 (3H), 2.23 (1H), 2.09 (1H).

### Example 254 2-[3,3-Difluoro-1-((S)-tetrahydro-furan-2-carbonyl)-piperidin-4-yloxy]-5-{2-[6-methoxy-5-(4-oxetan-3-yl-piperazin-1-yl)-pyridin-2-ylamino]-pyrimidin-4-yl}-benzonitrile

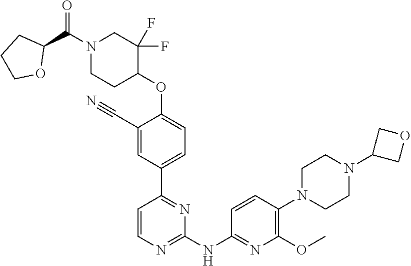 [SMILES: COc1nc(Nc2nccc(-c3ccc(OC4CCN(C(=O)[C@@H]5CCCO5)CC4(F)F)c(C#N)c3)n2)ccc1N1CCN(C2COC2)CC1]

The title compound (18.50 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-Tetrahydro-furan-2-carboxylic acid (32.70 mg) using Method A in 20% yield, m/z: 677 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.78 (1H), 4.59 (2H), 4.49 (1H), 3.92 (3H), 3.75 (2H), 3.66 (1H), 3.46 (1H), 3.04 (3H), 2.38 (3H), 2.06 (3H), 1.98 (1H).

### Example 255: 2-[1-((S)-2,2-Difluoro-cyclopropanecarbonyl)-3,3-difluoro-piperidin-4-yloxy]-5-{2-[6-methoxy-5-(4-oxetan-3-yl-piperazin-1-yl)-pyridin-2-ylamino]-pyrimidin-4-yl}-benzonitrile

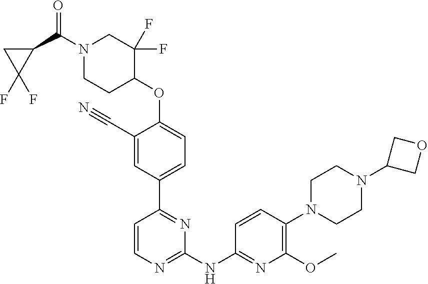 [SMILES: COc1nc(Nc2nccc(-c3ccc(OC4CCN(C(=O)[C@@H]5CC5(F)F)CC4(F)F)c(C#N)c3)n2)ccc1N1CCN(C2COC2)CC1]

The title compound (26.70 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-2,2-Difluoro-cyclopropanecarboxylic acid (33.70 mg) using Method A in 28% yield, m/z: 683 (M+H). ¹H NMR (DMSO-d6): 9.49 (1H), 8.61 (2H), 8.51 (1H), 7.78 (1H), 7.65 (1H), 7.56 (1H), 7.39 (1H), 5.40 (1H), 4.59 (2H), 4.49 (1H), 3.92 (3H), 3.45 (1H), 3.04 (3H), 2.40 (2H), 2.16 (1H), 1.90 (2H).

### Example 256: 2,2-[3,3-Difluoro-1-((S)-5-oxo-pyrrolidine-2-carbonyl)-piperidin-4-yloxy]-5-{2-[6-methoxy-5-(4-oxetan-3-yl-piperazin-1-yl)-pyridin-2-ylamino]-pyrimidin-4-yl}-benzonitrile

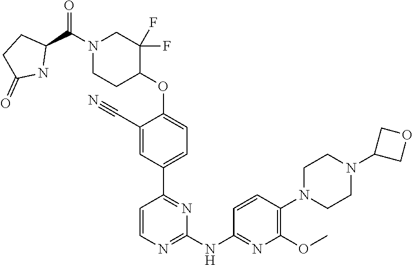 [SMILES: COc1nc(Nc2nccc(-c3ccc(OC4CCN(C(=O)[C@@H]5CCC(=O)N5)CC4(F)F)c(C#N)c3)n2)ccc1N1CCN(C2COC2)CC1]

The title compound (35.4 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-5-Oxo-pyrrolidine-2-carboxylic acid (35.70 mg) using Method A in 37% yield, m/z: 690 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H).

### Example 257: 2-[3,3-Difluoro-1-((R)-3-hydroxy-2-methyl-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

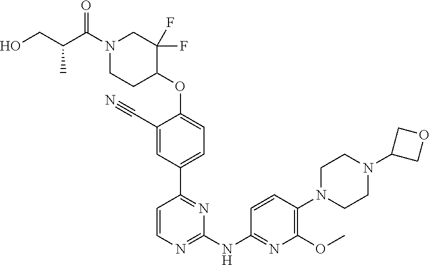 [SMILES: COc1nc(Nc2nccc(-c3ccc(OC4CCN(C(=O)[C@H](C)CO)CC4(F)F)c(C#N)c3)n2)ccc1N1CCN(C2COC2)CC1]

The title compound (24 mg) was synthesized using 2-(3,3-Difluoro-piperidin-4-yloxy)-5-[2-(pyridin-2-ylamino)-pyrimidin-4-yl]-benzonitrile hydrochloride (80 mg) and (R)-3-Hydroxy-2-methyl-propionic acid (28.79 mg) using Method A in 26% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.0 (3H).

### Example 258: 2-[3,3-Difluoro-1-((S)-2-hydroxy-butyryl)-piperidin-4-yloxy]-5-{2-[6-methoxy-5-(4-oxetan-3-yl-piperazin-1-yl)-pyridin-2-ylamino]-pyrimidin-4-yl}-benzonitrile

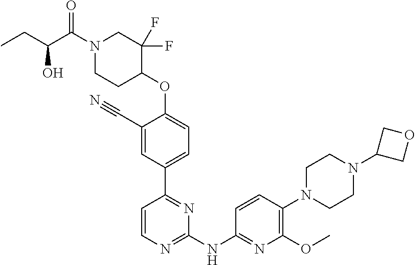 [SMILES: CC[C@H](O)C(=O)N1CCC(Oc2ccc(-c3ccnc(Nc4ccc(N5CCN(C6COC6)CC5)c(OC)n4)n3)cc2C#N)C(F)(F)C1]

The title compound (30.4 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-2-Hydroxy-butyric acid (28.78 mg) using Method A in 32% yield, m/z: 665 (M+H). ¹H NMR (DMSO-d6): 9.49 (1H), 8.61 (2H), 8.51 (1H), 7.78 (1H), 7.65 (1H), 7.56 (1H), 7.39 (1H), 5.38 (1H0, 5.14 (1H), 4.59 (2H), 4.49 (2H), 4.25 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.65 (1H), 1.52 (1H), 0.89 (3H).

### Example 259: 2-[3,3-Difluoro-1-(2-fluoro-propionyl)-piperidin-4-yloxy]-5-{2-[6-methoxy-5-(4-oxetan-3-yl-piperazin-1-yl)-pyridin-2-ylamino]-pyrimidin-4-yl}-benzonitrile

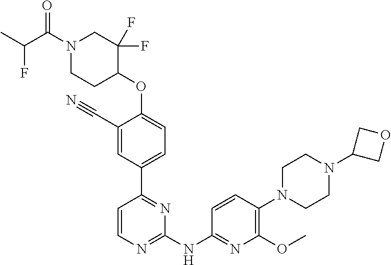 [SMILES: COc1nc(Nc2nccc(-c3ccc(OC4CCN(C(=O)C(C)F)CC4(F)F)c(C#N)c3)n2)ccc1N1CCN(C2COC2)CC1]

The title compound (34.1 mg) was synthesized using 2-(3,3-Difluoro-piperidin-4-yloxy)-5-[2-(pyridin-2-ylamino)-pyrimidin-4-yl]-benzonitrile hydrochloride (80 mg) and 2-Fluoro-propionic acid (25.43 mg) using Method A in 36% yield, m/z: 663 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.30 (2H), 1.90 (1H), 1.86 (1H), 1.45-1.42 (3H).

### Example 260: 2-[1-((S)-2,3-Dihydroxy-propionyl)-3,3-difluoro-piperidin-4-yloxy]-5-{2-[6-methoxy-5-(4-oxetan-3-yl-piperazin-1-yl)-pyridin-2-ylamino]-pyrimidin-4-yl}-benzonitrile

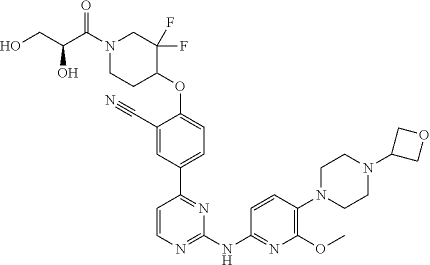 [SMILES: COc1nc(Nc2nccc(-c3ccc(OC4CCN(C(=O)[C@@H](O)CO)CC4(F)F)c(C#N)c3)n2)ccc1N1CCN(C2COC2)CC1]

The title compound (17.8 mg) was synthesized using 2-(3,3-Difluoro-piperidin-4-yloxy)-5-[2-(pyridin-2-ylamino)-pyrimidin-4-yl]-benzonitrile hydrochloride (80 mg) and (S)-2,3-Dihydroxy-propionic acid (29.33 mg) using Method A in 19% yield, m/z: 667 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.59 (2H), 4.49 (1H), 4.45 (1H), 3.92 (3H), 3.45 (2H), 3.17 (1H), 3.04 (1H), 2.89 (2H), 2.74 (2H), 2.58 (1H), 2.38 (1H), 2.40 (2H) 2.30 (2H), 1.90 (1H), 1.86 (1H).

### Example 261: 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile

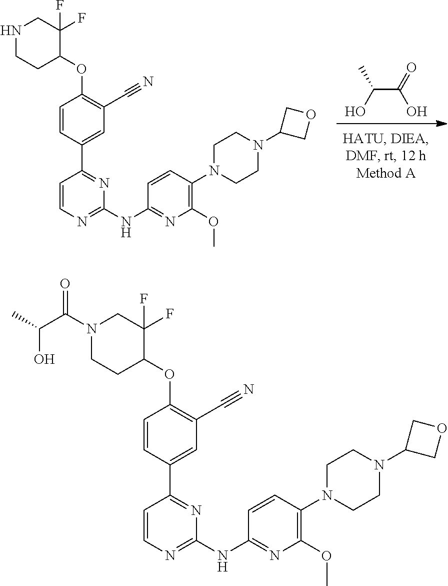

The title compound was prepared from 2-[(3,3-difluoropiperidin-4-yl)oxy]-5-[2-([6-methoxy-5-[4-(oxetan-3-yl)piperazin-1-yl]pyridin-2-yl]amino)pyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 30% to 40% gradient in 8 min; detector, UV 254 nm. 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]benzonitrile was obtained as a light yellow solid (32 mg, 20%). HPLC: 99.6% purity, RT=4.23 min. MS: m/z=651.2 [M+H]⁺. ¹H NMR (400 MHz, DMSO-d₆, ppm) δ9.38 (s, 1H), 8.64-8.57 (m, 2H), 8.57-8.49 (m, 1H), 7.77-7.71 (m, 1H), 7.71-7.63 (m, 1H), 7.57-7.51 (m, 1H), 7.31-7.24 (m, 1H), 5.41-5.36 (m, 1H), 5.26-5.18 (m, 1H), 4.62-4.38 (m, 5H), 4.29-3.94 (m, 2H), 3.90 (s, 3H), 3.87-3.53 (m, 2H), 3.52-3.42 (m, 1H), 3.01-2.96 (m, 4H), 2.43-2.39 (m, 4H), 2.23-1.79 (m, 2H), 1.23 (d, J=6.5 Hz, 3H).

### Example 262: 2-[1-((S)-2,3-Dihydroxy-propionyl)-3,3-difluoro-piperidin-4-yloxy]-5-{2-[6-methoxy-5-((R)-3-methyl-4-oxetan-3-yl-piperazin-1-yl)-pyridin-2-ylamino]-pyrimidin-4-yl}-benzonitrile

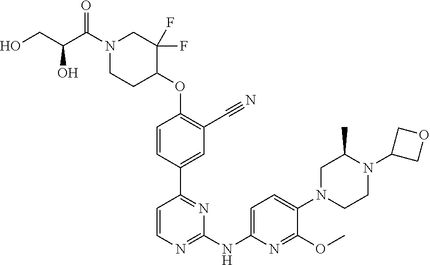 [SMILES: COc1nc(Nc2nccc(-c3ccc(OC4CCN(C(=O)[C@@H](O)CO)CC4(F)F)c(C#N)c3)n2)ccc1N1CCN(C2COC2)[C@H](C)C1]

The title compound (19.1 mg) was synthesized using 2-(3,3-Difluoro-piperidin-4-yloxy)-5-{2-[6-methoxy-5-((R)-3-methyl-4-oxetan-3-yl-piperazin-1-yl)-pyridin-2-ylamino]-pyrimidin-4-yl}-benzonitrile (100 mg) and (S)-2,3-Dihydroxy-propionic acid (48.40 mg) using Method A in 17% yield, m/z: 681 (M+H). ¹H NMR (DMSO-d6): 10.4 (1H), 8.72 (2H), 6.64 (1H), 8.54 (1H), 8.41 (1h), 7.99 (2H), 7.76 (1H), 7.68 (1H), 5.39 (1H), 5.23 (1H), 4.49 (1H), 4.10 (1H), 4.06 (1H), 3.90 93H), 3.65 (1H), 2.18 (1H), 2.00 (1H). 1.23 (3H).

### Example 263: 2-(3,3-Difluoro-4-methyl-piperidin-4-yloxy)-5-{2-[6-methoxy-5-(4-oxetan-3-yl-piperazin-1-yl)-pyridin-2-ylamino]-pyrimidin-4-yl}-benzonitrile

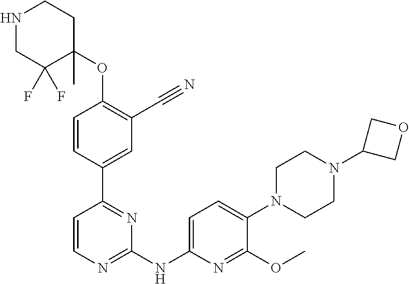 [SMILES: COc1nc(Nc2nccc(-c3ccc(OC4(C)CCNCC4(F)F)c(C#N)c3)n2)ccc1N1CCN(C2COC2)CC1]

The title compound (900 mg) was synthesized using 4-(2-Cyano-4-{2-[6-methoxy-5-(4-oxetan-3-yl-piperazin-1-yl)-pyridin-2-ylamino]-pyrimidin-4-yl}-phenoxy)-3,3-difluoro-4-methy 1-piperidine-1-carboxylic acid tert-butyl ester (1300 mg) and TFA (4 mL) using Method 17 in 77% yield, m/z: 593 (M+H). ¹H NMR (DMSO-d6): 9.47 (1H), 8.62 (2H), 8.52 (1H), 7.81 (1H), 7.65 (1H), 7.59 (1H), 5.20 (1H), 4.56 (2H), 4.45 (2H), 3.90 (3H), 3.42 (1H), 3.18 (1H), 2.91 (2H), 2.81 (1H), 2.73 (1H), 2.07 (1H), 1.86 (3H), 1.70 (3H).

### Example 264: 2-[1-((S)-2,3-Dihydroxy-propionyl)-3,3-difluoro-4-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

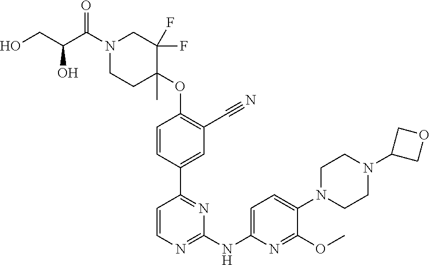 [SMILES: COc1nc(Nc2nccc(-c3ccc(OC4(C)CCN(C(=O)[C@@H](O)CO)CC4(F)F)c(C#N)c3)n2)ccc1N1CCN(C2COC2)CC1]

The title compound (30.2 mg) was synthesized using 2-(3,3-Difluoro-4-methyl-piperidin-4-yloxy)-5-{2-[6-methoxy-5-(4-oxetan-3-yl-piperazin-1-yl)-pyridin-2-ylamino]-pyrimidin-4-yl}-benzonitrile (100 mg) and (S)-2,3-Dihydroxy-propionic acid (35.80 mg) using Method A in 26% yield, m/z: 681 (M+H). ¹H NMR (DMSO-d6): 9.37 (1H), 8.62 (2H), 8.54 (1H), 7.76 (1H), 7.59 (1H), 7.51 (1H), 7.30 (1H), 4.87 (1H), 4.58 (2H), 4.46 (2H), 3.90 (3H), 3.45 (2H), 2.99 (3H), 2.43 (2H), 1.77 (2H), 1.59 (2H), 1.32 (1H), 1.21 (2H).

### Example 265: 2-[3,3-Difluoro-1-((S)-2-hydroxy-propionyl)-4-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

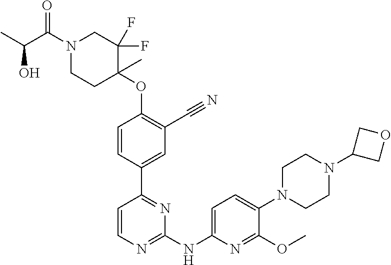 [SMILES: COc1nc(Nc2nccc(-c3ccc(OC4(C)CCN(C(=O)[C@H](C)O)CC4(F)F)c(C#N)c3)n2)ccc1N1CCN(C2COC2)CC1]

The title compound (71.6 mg) was synthesized using 2-(3,3-Difluoro-4-methyl-piperidin-4-yloxy)-5-{2-[6-methoxy-5-(4-oxetan-3-yl-piperazin-1-yl)-pyridin-2-ylamino]-pyrimidin-4-yl}-benzonitrile (100 mg) and (R)-2-Hydroxy-propionic acid (32.50 mg) using Method A in 63% yield, m/z: 665 (M+H). ¹H NMR (DMSO-d6): 9.37 (1H), 8.62 (2H), 8.54 (1H), 7.76 (1H), 7.59 (1H), 7.51 (1H), 7.30 (1H), 4.87 (1H), 4.58 (2H), 4.46 (2H), 3.90 (3H), 3.45 (2H), 2.99 (3H), 2.43 (2H), 1.77 (2H), 1.59 (2H), 1.32 (1H), 1.21 (2H).

### Example 266: 2-[1-((S)-2-Hydroxy-propionyl)-2-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

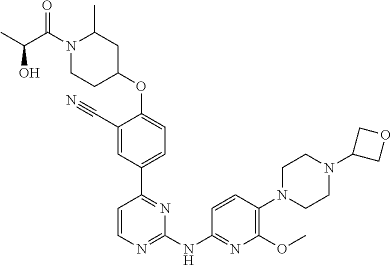 [SMILES: COc1nc(Nc2nccc(-c3ccc(OC4CCN(C(=O)[C@H](C)O)C(C)C4)c(C#N)c3)n2)ccc1N1CCN(C2COC2)CC1]

The title compound (5.70 mg) was synthesized using 5-{2-[6-Methoxy-5-(4-oxetan-3-yl-piperazin-1-yl)-pyridin-2-ylamino]-pyrimidin-4-yl}-2-(2-methyl-piperidin-4-yloxy)-benzonitrile (80 mg) and (S)-2-hydroxy-propionic acid (28.73 mg) using Method A in 5% yield, m/z: 629 (M+H). ¹H NMR (DMSO-d6): 9.49 (1H), 8.61 (2H), 8.51 (1H), 7.78 (1H), 7.65 (1H), 7.56 (1H), 7.39 (1H), 5.24 (1H), 4.78 (1H), 4.59 (2H), 4.49 (1H), 3.92 (3H), 3.75 (2H), 3.66 (1H), 3.46 (1H), 3.04 (3H), 2.38 (3H), 2.06 (3H), 1.38 (2H).

### Example 267: 5-{2-[6-Methoxy-5-(4-oxetan-3-yl-piperazin-1-yl)-pyridin-2-ylamino]-pyrimidin-4-yl}-2-(4-methyl-piperidin-4-yloxy)-benzonitrile

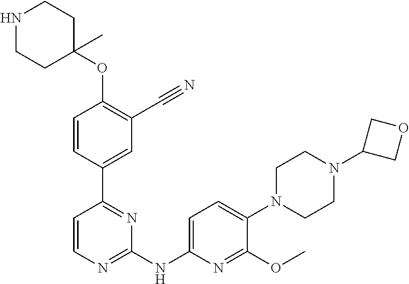 [SMILES: COc1nc(Nc2nccc(-c3ccc(OC4(C)CCNCC4)c(C#N)c3)n2)ccc1N1CCN(C2COC2)CC1]

The title compound (880 mg) was synthesized using 4-(2-Cyano-4-{2-[6-methoxy-5-(4-oxetan-3-yl-piperazin-1-yl)-pyridin-2-ylamino]-pyrimidin-4-yl}-phenoxy)-4-methyl-piperidine-1-carboxylic acid tert-butyl ester (1600 mg) and TFA (4 mL) using Method 17 in 62% yield, m/z: 557 (M+H). ¹H NMR (DMSO-d6): 9.47 (1H), 8.62 (2H), 8.52 (1H), 7.81 (1H), 7.65 (1H), 7.59 (1H), 5.20 (1H), 4.56 (2H), 4.45 (2H), 3.90 (3H), 3.42 (1H), 3.18 (1H), 2.91 (2H), 2.81 (1H), 2.73 (1H), 2.07 (1H), 1.86 (3H), 1.70 (3H).

### Example 268: 2-[1-((S)-2-Hydroxy-propionyl)-4-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

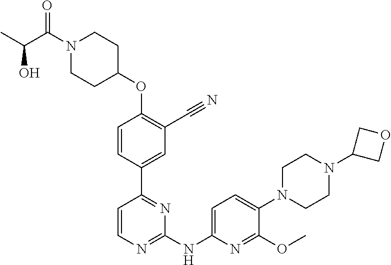 [SMILES: COc1nc(Nc2nccc(-c3ccc(OC4CCN(C(=O)[C@H](C)O)CC4)c(C#N)c3)n2)ccc1N1CCN(C2COC2)CC1]

The title compound (28.6 mg) was synthesized using 5-{2-[6-Methoxy-5-(4-oxetan-3-yl-piperazin-1-yl)-pyridin-2-ylamino]-pyrimidin-4-yl}-2-(4-methyl-piperidin-4-yloxy)-benzonitrile (100 mg) and (R)-2-Hydroxy-propionic acid (32.50 mg) using Method A in 23% yield, m/z: 629 (M+H). ¹H NMR (DMSO-d6): 9.37 (1H), 8.62 (2H), 8.54 (1H), 7.76 (1H), 7.59 (1H), 7.51 (1H), 7.30 (1H), 4.87 (1H), 4.58 (2H), 4.46 (2H), 3.90 (3H), 3.45 (2H), 2.99 (3H), 2.43 (3H), 2.18 (2H), 1.77 (2H), 1.59 (2H), 1.32 (1H), 1.21 (2H).

### Example 269: 2-[1-((S)-2,3-Dihydroxy-propionyl)-4-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

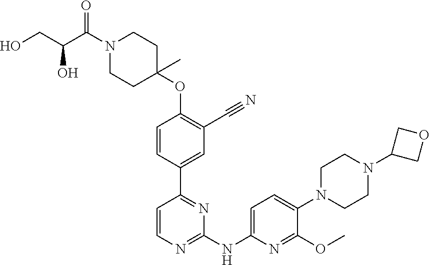 [SMILES: COc1nc(Nc2nccc(-c3ccc(OC4(C)CCN(C(=O)[C@@H](O)CO)CC4)c(C#N)c3)n2)ccc1N1CCN(C2COC2)CC1]

The title compound (17.40 mg) was synthesized using 5-{2-[6-Methoxy-5-(4-oxetan-3-yl-piperazin-1-yl)-pyridin-2-ylamino]-pyrimidin-4-yl}-2-(4-methyl-piperidin-4-yloxy)-benzonitrile (100 mg) and (S)-2,3-Dihydroxy-propionic acid (32.50 mg) using Method A in 15% yield, m/z: 645 (M+H). ¹H NMR (DMSO-d6): 9.37 (1H), 8.62 (2H), 8.54 (1H), 7.76 (1H), 7.59 (1H), 7.51 (1H), 7.30 (1H), 4.87 (1H), 4.58 (2H), 4.46 (2H), 3.90 (3H), 3.45 (2H), 2.99 (3H), 2.43 (2H), 2.18 (2H), 1.77 (2H), 1.59 (2H), 1.32 (1H), 1.21 (2H).

### Example 270: 5-{2-[6-Methoxy-5-(4-oxetan-3-yl-piperazin-1-yl)-pyridin-2-ylamino]-pyrimidin-4-yl}-2-(3-methyl-piperidin-4-yloxy)-benzonitrile

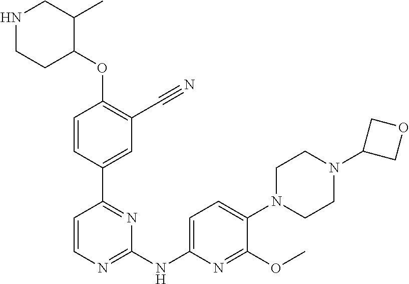 [SMILES: COc1nc(Nc2nccc(-c3ccc(OC4CCNCC4C)c(C#N)c3)n2)ccc1N1CCN(C2COC2)CC1]

The title compound (400 mg) was synthesized using 4-(2-Cyano-4-{2-[6-methoxy-5-(4-oxetan-3-yl-piperazin-1-yl)-pyridin-2-ylamino]-pyrimidin-4-yl}-phenoxy)-3-methyl-piperidine-1-carboxylic acid tert-butyl ester (1300 mg) and TFA (4 mL) using Method 17 in 36% yield, m/z: 557 (M+H). ¹H NMR (DMSO-d6): 9.36 (1H), 8.58 (2H), 8.47 (1H), 7.76 (1H), 7.53 (2H), 7.29 (1H), 4.53 (2H), 4.48 (2H), 4.33 (1H), 3.90 (3H), 3.43 (1H), 2.99 (3H), 2.73 (2H), 2.41 (2H), 2.05 (1H), 1.82 (1H), 1.41 (1H), 0.93 (2H).

### Example 271: 2-[1-((S)-2-Hydroxy-propionyl)-3-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

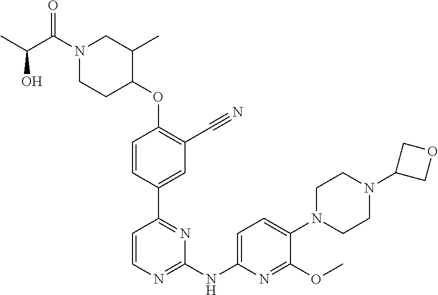 [SMILES: COc1nc(Nc2nccc(-c3ccc(OC4CCN(C(=O)[C@H](C)O)CC4C)c(C#N)c3)n2)ccc1N1CCN(C2COC2)CC1]

The title compound (53.1 mg) was synthesized using 5-{2-[6-Methoxy-5-(4-oxetan-3-yl-piperazin-1-yl)-pyridin-2-ylamino]-pyrimidin-4-yl}-2-(3-methyl-piperidin-4-yloxy)-benzonitrile (100 mg) and (S)-2-Hydroxy-propionic acid (32.40 mg) using Method A in 46% yield, m/z: 629 (M+H). ¹H NMR (DMSO-d6): 9.66 (1H), 8.58 (2H), 8.52 (1H), 7.86 (1H), 7.67 (1H), 7.53 (2H), 7.29 (1H), 4.77 (1H), 4.93 (2H), 4.71 (2H), 4.55 (2H), 4.48 (2H), 4.38 (1H), 4.08 (1H), 3.91 (3H), 3.48-3.52 (4H), 3.30 (1H), 2.99 (4H), 2.42 (2H), 2.18 (2H), 1.89 (2H).1.03 (3H).

### Example 272: 2-[1-((S)-2,3-Dihydroxy-propionyl)-3-methyl-piperidin-4-yloxy]-5-{2-[6-methoxy-5-(4-oxetan-3-yl-piperazin-1-yl)-pyridin-2-ylamino]-pyrimidin-4-yl}-benzonitrile

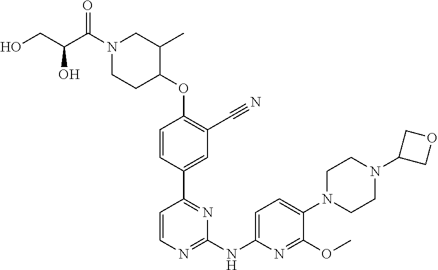 [SMILES: COc1nc(Nc2nccc(-c3ccc(OC4CCN(C(=O)[C@@H](O)CO)CC4C)c(C#N)c3)n2)ccc1N1CCN(C2COC2)CC1]

The title compound (26.20 mg) was synthesized using 5-{2-[6-Methoxy-5-(4-oxetan-3-yl-piperazin-1-yl)-pyridin-2-ylamino]-pyrimidin-4-yl}-2-(3-methyl-piperidin-4-yloxy)-benzonitrile (100 mg) and (S)-2,3-Dihydroxy-propionic acid (32.40 mg) using Method A in 23% yield, m/z: 645 (M+H). ¹H NMR (DMSO-d6): 9.66 (1H), 8.58 (2H), 8.52 (1H), 7.86 (1H), 7.67 (1H), 7.53 (2H), 7.29 (1H), 4.77 (1H), 4.93 (2H), 4.71 (2H), 4.55 (2H), 4.48 (2H), 4.38 (1H), 4.08 (1H), 3.91 (3H), 3.48 (2H), 3.30 (1H), 2.99 (4H), 2.42 (2H), 2.18 (2H), 1.89 (2H), 1.03 (3H).

### Example 273: 2-[[1-(5-methyl-1H-1,2,4-triazole-3-carbonyl)piperidin-4-yl]oxy]-5-[2-([4-[4-(oxetan-3-yl)piperazin-1-yl]phenyl]amino)pyrimidin-4-yl]benzonitrile

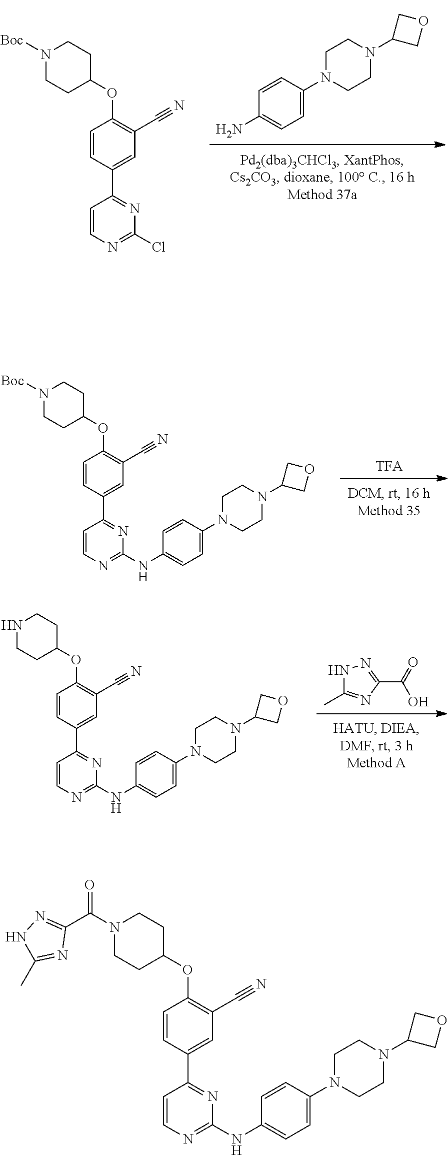

5-[2-([4-[4-(oxetan-3-yl)piperazin-1-yl]phenyl]amino)pyrimidin-4-yl]-2-(piperidin-4-yloxy)benzonitrile: The title compound was prepared from tert-butyl 3-[4-(2-chloropyrimidin-4-yl)-2-cyanophenoxy]piperidine-1-carboxylate and 4-[4-(oxetan-3-yl)piperazin-1-yl]aniline using Method 37a and 35. The final product was purified by prep-HPLC under the following condition: column, XBridge Prep OBD C18 Column, 150×30 mm, 5 um; mobile phase, acetonitrile in water (with 10 mmol/L NH₁HCO₃ and 0.1% NH₃.H₂O), 20% to 50% gradient in 8 min; detector, UV 254 nm. 5-[2-([4-[4-(oxetan-3-yl)piperazin-1-yl]phenyl]amino)pyrimidin-4-yl]-2-(piperidin-4-yloxy)benzonitrile was obtained as a light yellow solid (3 mg, 2.6% for 2 steps). HPLC: 97.6% purity, RT=3.36 min. MS: m/z=512.2 [M+H]⁺. ¹H NMR (300 MHz, DMSO-d₆, ppm) δ9.40 (s, 1H), 8.51-8.43 (m, 2H), 8.43-8.34 (m, 1H), 7.64-7.54 (m, 2H), 7.52-7.43 (m, 1H), 7.39-7.31 (m, 1H), 6.95-6.86 (m, 2H), 4.78-4.72 (m, 1H), 4.55 (t, J=6.5 Hz, 2H), 4.46 (t, J=6.0 Hz, 2H), 3.50-3.36 (m, 1H), 3.13-3.04 (m, 4H), 3.00-2.90 (m, 2H), 2.66-2.48 (m, 2H), 2.44-2.35 (m, 4H), 1.99-1.88 (m, 2H), 1.61-1.48 (m, 2H).

2-[[1-(5-methyl-1H-1,2,4-triazole-3-carbonyl)piperidin-4-yl]oxy]-5-[2-([4-[4-(oxetan-3-yl)piperazin-1-yl]phenyl]amino)pyrimidin-4-yl]benzonitrile: 2-[[1-(5-methyl-1H-1,2,4-triazole-3-carbonyl)piperidin-4-yl]oxy]-5-[2-([4-[4-(oxetan-3-yl)piperazin-1-yl]phenyl]amino)pyrimidin-4-yl]benzonitrile was prepared from 5-[2-([4-[4-(oxetan-3-yl)piperazin-1-yl]phenyl]amino)pyrimidin-4-yl]-2-(piperidin-4-yloxy)benzonitrile and 5-methyl-1H-1,2,4-triazole-3-carboxylic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 18% to 48% gradient in 8 min; detector, UV 254 nm. 2-[[1-(5-methyl-1H-1,2,4-triazole-3-carbonyl)piperidin-4-yl]oxy]-5-[2-([4-[4-(oxetan-3-yl)piperazin-1-yl]phenyl]amino)pyrimidin-4-yl]benzonitrile was obtained as a light yellow solid (31 mg, 22%). HPLC: 99.8% purity, RT=3.91 min. MS: m/z=621.2 [M+H]⁺. ¹H NMR (300 MHz, DMSO-d₆.ppm) δ 14.01 (br s, 1H), 9.41 (s, 1H), 8.53-8.38 (m, 3H), 7.67-7.49 (m, 3H), 7.41-7.33 (m, 1H), 6.95-6.86 (m, 2H), 5.06-5.00 (m, 1H), 4.55 (t, J=6.5 Hz, 2H), 4.46 (t, J=6.0 Hz, 2H), 4.09-3.54 (m, 4H), 3.50-3.36 (m, 1H), 3.13-3.04 (m, 4H), 2.44-2.37 (m, 4H), 2.35 (s, 3H), 2.14-1.91 (m, 2H), 1.84-1.62 (m, 2H).

### Example 274: 2-[[1-(2-methyl-1H-imidazole-4-carbonyl)piperidin-4-yl]oxy]-5-[2-([4-[4-(oxetan-3-yl)piperazin-1-yl]phenyl]amino)pyrimidin-4-yl]benzonitrile

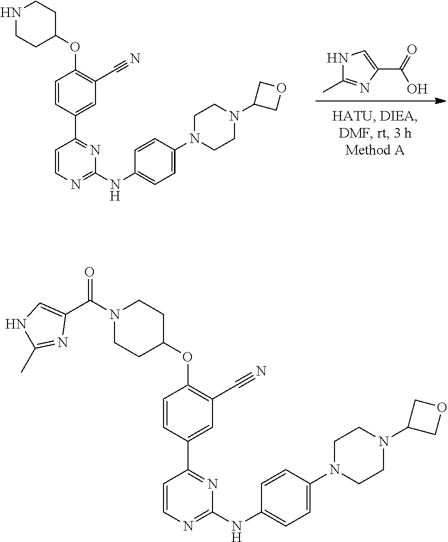

The title compound was prepared from 5-[2-([4-[4-(oxetan-3-yl)piperazin-1-yl]phenyl]amino)pyrimidin-4-yl]-2-(piperidin-4-yloxy)benzonitrile and 2-methyl-1H-imidazole-4-carboxylic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 20% to 49% gradient in 8 min; detector, UV 254 nm. 2-[[1-(2-methyl-1H-imidazole-4-carbonyl)piperidin-4-yl]oxy]-5-[2-([4-[4-(oxetan-3-yl)piperazin-1-yl]phenyl]amino)pyrimidin-4-yl]benzonitrile was obtained as a light yellow solid (17 mg, 12%). HPLC: 95.4% purity, RT=3.61 min. MS: m/z=620.2 [M+H]⁺. ¹H NMR (300 MHz, DMSO-d₆.ppm) δ12.11 (s, 1H), 9.41 (s, 1H), 8.58-8.35 (m, 3H), 7.67-7.45 (m, 4H), 7.41-7.33 (m, 1H), 6.95-6.86 (m, 2H), 5.03-4.97 (m, 1H), 4.55 (t, J=6.5 Hz, 2H), 4.46 (t, J=6.0 Hz, 2H), 4.22-3.53 (m, 4H), 3.50-3.38 (m, 1H), 3.12-3.04 (m, 4H), 2.44-2.35 (m, 4H), 2.27 (s, 3H), 2.04-1.98 (m, 2H), 1.73-1.64 (m, 2H).

### Example 275: 2-([3,3-difluoro-1-[(2S)-2-hydroxypropanoyl]piperidin-4-yl]oxy)-5-(2-[[6-methoxy-5-(morpholin-4-yl)pyridin-2-yl]amino]pyrimidin-4-yl)benzonitrile

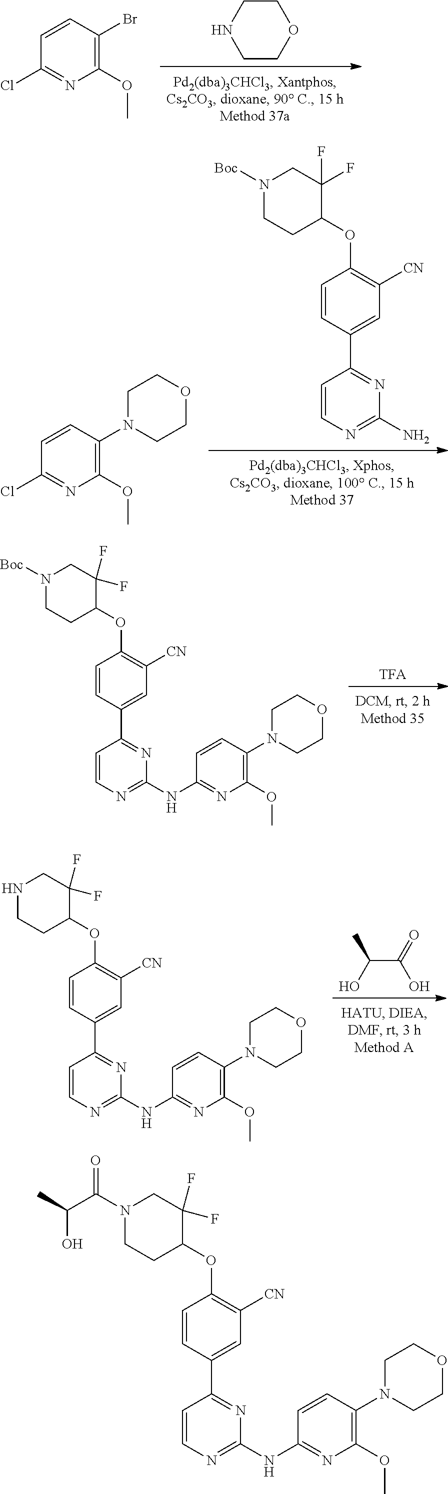

The title compound was prepared from morpholine, 3-bromo-6-chloro-2-methoxypyridine, tert-butyl 4-(4-(2-aminopyrimidin-4-yl)-2-cyanophenoxy)-3,3-difluoropiperidine-1-carboxylate and (S)-2-hydroxypropanoic acid using Method 37a, 37, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃), 30% to 60% gradient in 8 min; detector, UV 254 nm. 2-([3,3-difluoro-1-[(2S)-2-hydroxypropanoyl]piperidin-4-yl]oxy)-5-(2-[[6-methoxy-5-(morpholin-4-yl)pyridin-2-yl]amino]pyrimidin-4-yl)benzonitrile was obtained as an yellow solid (33 mg, 12% for 4 steps). HPLC: 97.3% purity, RT=8.45 min. MS: m/z=596.2 [M+H]⁺. ¹H NMR (300 MHz, DMSO-d₆, ppm) δ 9.41 (s, 1H), 8.61-8.57 (m, 2H), 8.53-8.50 (m, 1H), 7.75 (d, J=8.4 Hz, 1H), 7.66 (d, J=9.1 Hz, 1H), 7.53 (d, J=5.1 Hz, 1H), 7.26 (d, J=8.4 Hz, 1H), 5.38-5.36 (m, 1H), 5.24-5.20 (m, 1H), 4.51-4.45 (m, 1H), 4.31-3.41 (m, 11H), 2.94-2.91 (m, 4H), 2.50-1.81 (m, 2H), 1.24-1.20 (m, 3H).

### Example 276: 2-[1-((S)-2,3-Dihydroxy-propionyl)-3,3-difluoro-piperidin-4-yloxy]-5-{2-[3-(l-oxetan-3-yl-piperidin-4-yl)-phenylamino]-pyrimidin-4-yl}-benzonitrile

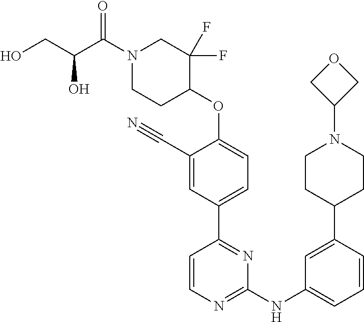 [SMILES: N#Cc1cc(-c2ccnc(Nc3cccc(C4CCN(C5COC5)CC4)c3)n2)ccc1OC1CCN(C(=O)[C@@H](O)CO)CC1(F)F]

The title compound (62.8 mg) was synthesized using 2-(3,3-Difluoro-piperidin-4-yloxy)-5-{2-[3-(1-oxetan-3-yl-piperidin-4-yl)-phenylamino]-pyrimidin-4-yl}-benzonitrile (100 mg) and (S)-2,3-Dihydroxy-propionic acid (48.40 mg) using Method A in 52% yield, m/z: 635 (M+H). ¹H NMR (DMSO-d6): 9.66 (1H), 8.58 (2H), 8.52 (1H), 7.86 (1H), 7.67 (1H), 7.53 (1H), 7.29 (1H), 6.90 (1H), 5.42 (1H), 5.27 (1H), 4.77 (1H), 4.53 (2H), 4.48 (2H), 4.38 (1H), 3.86 (1H), 3.56 (2H), 3.30 (1H), 2.83 (2H), 2.11 (1H), 1.89 (3H), 1.70 (2H).

### Example 277: 2-([1-[(2S)-2-hydroxypropanoyl]azetidin-3-yl]methoxy)-5-[2-([4-[4-(oxetan-3-yl)piperazin-1-yl]phenyl]amino)pyrimidin-4-yl]benzonitrile

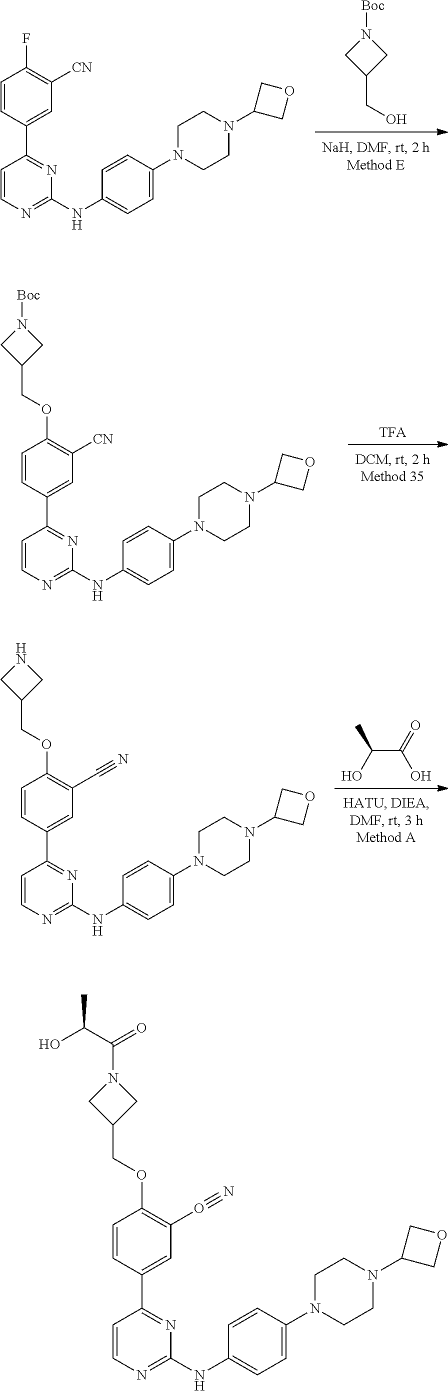

2-[(azetidin-3-yl)methoxy]-5-[2-([4-[4-(oxetan-3-yl)piperazin-1-yl]phenyl]amino)pyrimidin-4-yl]benzonitrile: The title compound was prepared from 2-fluoro-5-(2-(4-(4-(oxetan-3-yl)piperazin-1-yl)phenylamino)pyrimidin-4-yl)benzonitrile and tert-butyl 3-(hydroxymethyl)azetidine-1-carboxylate using Method E and 35. The final product was purified by prep-HPLC under the following conditions: column, XBridge Prep OBD C18 Column, 150×30 mm, 5 um; mobile phase, acetonitrile in water (with 10 mmol/L NH₄HCO₃), 22% to 42% gradient in 8 min; detector, UV 254 nm. 2-[(azetidin-3-yl)methoxy]-5-[2-([4-[4-(oxetan-3-yl)piperazin-1-yl]phenyl]amino)pyrimidin-4-yl]benzonitrile was obtained as an yellow solid (3 mg, 32% for 2 steps). HPLC: 90.0% purity, RT=3.26 min. MS: m/z=498.3 [M+H]⁺. ¹H NMR (300 MHz, DMSO-d6, ppm) δ 9.41 (s, 1H), 8.52-8.40 (m, 3H), 7.63-7.60 (m, 2H), 7.50-7.41 (m, 1H), 7.41-7.33 (m, 1H), 6.95-6.86 (m, 2H), 4.60-4.52 (m, 2H), 4.51-4.42 (m, 2H), 4.42-4.33 (m, 2H), 3.61-3.58 (m, 1H), 3.32-3.20 (m, 4H), 3.12-3.06 (m, 5H), 2.43-2.37 (m, 4H).

2-([1-[(2S)-2-hydroxypropanoyl]azetidin-3-yl]methoxy)-5-[2-([4-[4-(oxetan-3-yl)piperazin-1-yl]phenyl]amino)pyrimidin-4-yl]benzonitrile: The title compound was prepared from 2-fluoro-5-(2-(4-(4-(oxetan-3-yl)piperazin-1-yl)phenylamino)pyrimidin-4-yl)benzonitrile, tert-butyl 3-(hydroxymethyl)azetidine-1-carboxylate and (S)-2-hydroxypropanoic acid using Method E, 35 and A. The final product was purified by prep-HPLC under the following conditions: column, XBridge Prep OBD C18 Column, 150×30 mm, 5 um; mobile phase, acetonitrile in water (with 10 mmol/L NH₁HCO₃), 18% to 35% gradient in 8 min; detector, UV 254 nm. 2-([1-[(2S)-2-hydroxypropanoyl]azetidin-3-yl]methoxy)-5-[2-([4-[4-(oxetan-3-yl)piperazin-1-yl]phenyl]amino)pyrimidin-4-yl]benzonitrile was obtained as an yellow solid (22 mg, 30% for 3 steps). HPLC: 98.3% purity, RT=6.53 min. MS: m/z=570.3 [M+H]⁺. ¹H NMR (300 MHz, Methanol-d₄, ppm) δ 8.61-8.53 (m, 2H), 8.36-8.27 (m, 1H), 7.67 (d, J=6.8 Hz, 1H), 7.53-7.42 (m, 3H), 7.30-7.19 (m, 2H), 5.02-4.86 (m, 5H), 4.64-4.52 (m, 2H), 4.50-4.42 (m, 2H), 4.41-4.29 (m, 2H), 4.26-4.24 (m, 1H), 4.03-3.99 (m, 1H), 3.69-3.63 (m, 4H), 3.48-3.42 (m, 5H), 1.36 (d, J=6.8 Hz, 3H).

### Example 278: 2-([1-[(2R)-2-hydroxypropanoyl]azetidin-3-yl]methoxy)-5-[2-([4-[4-(oxetan-3-yl)piperazin-1-yl]phenyl]amino)pyrimidin-4-yl]benzonitrile

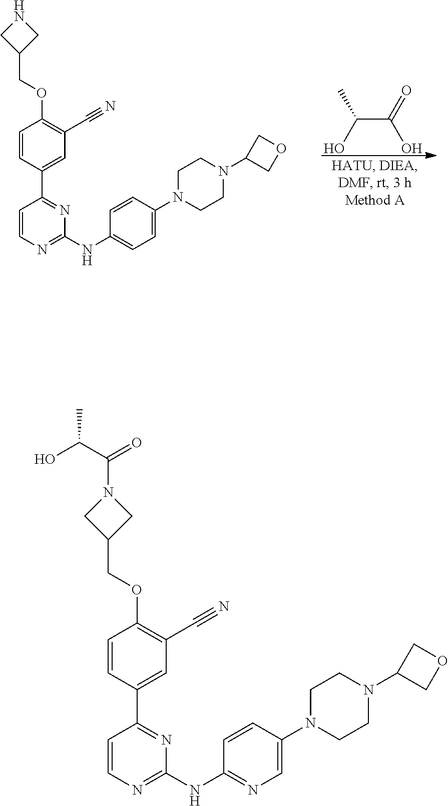

The title compound was prepared from 2-(azetidin-3-ylmethoxy)-5-(2-(4-(4-(oxetan-3-yl)piperazin-1-yl)phenylamino)pyrimidin-4-yl)benzonitrile and (R)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₄HCO₃), 18% to 35% gradient in 8 min; detector, UV 254 nm. 2-([1-[(2R)-2-hydroxypropanoyl]azetidin-3-yl]methoxy)-5-[2-([4-[4-(oxetan-3-yl)piperazin-1-yl]phenyl]amino)pyrimidin-4-yl]benzonitrile was obtained as an yellow solid (15 mg, 42%). HPLC: 99.4% purity, RT=3.77 min. MS: m/z=570.3 [M+H]⁺. ¹H NMR (300 MHz, DMSO-d₆, ppm) δ 9.42 (s, 1H), 8.53-8.40 (m, 3H), 7.60 (d, J=8.9 Hz, 2H), 7.50-7.34 (m, 2H), 6.91 (d, J=9.0 Hz, 2H), 5.06-4.96 (m, 1H), 4.61-4.36 (m, 7H), 4.19-3.92 (m, 2H), 3.77-3.68 (m, 1H), 3.48-3.38 (m, 1H), 3.39-3.35 (m, 1H), 3.13-3.04 (m, 5H), 2.44-2.37 (m, 4H), 1.17 (d, 7=6.7 Hz, 3H).

### Example 279: 2-([1-[(2S)-2-hydroxypropanoyl]azetidin-3-yl]methoxy)-5-[2-([6-methoxy-5-[4-(oxetan-3-yl)piperazin-1-yl]pyridin-2-yl]amino)pyrimidin-4-yl]benzonitrile

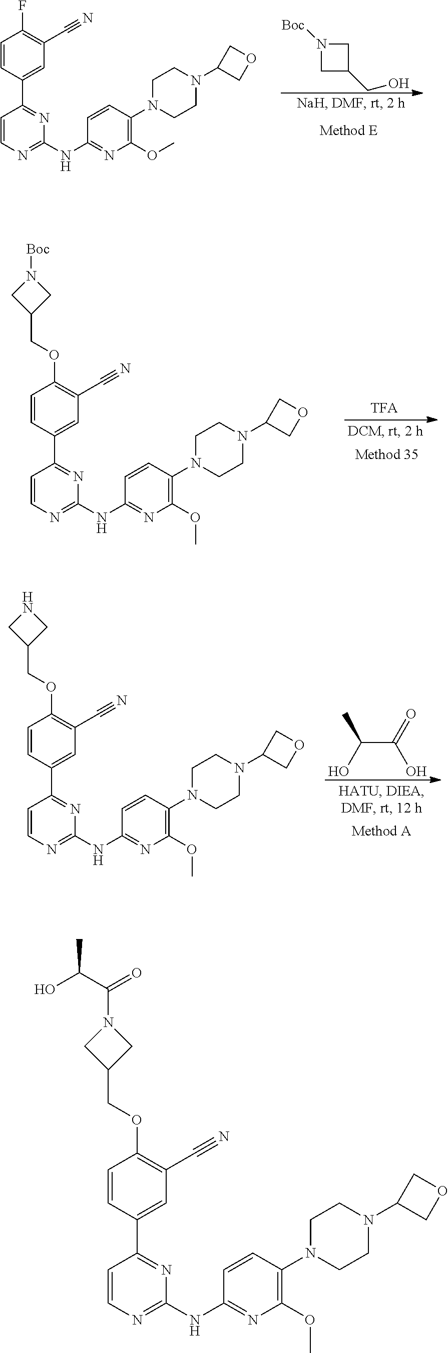

2-[(azetidin-3-yl)methoxy]-5-[2-([6-methoxy-5-[4-(oxetan-3-yl)piperazin-1-yl]pyridin-2-yl]amino)pyrimidin-4-yl]benzonitrile: The title compound was prepared from 2-fluoro-5-(2-(6-methoxy-5-(4-(oxetan-3-yl)piperazin-1-yl)pyridin-2-ylamino)pyrimidin-4-yl)benzonitrile and tert-butyl 3-(hydroxymethyl)azetidine-1-carboxylate using Method E and 35. The final product was purified by prep-HPLC under the following conditions: column, XBridge Prep OBD C18 Column, 150×30 mm, 5 um; mobile phase, acetonitrile in water (with 10 mmol/L NH₄HCO₃), 20% to 50% gradient in 8 min; detector, UV 254 nm. 2-[(azetidin-3-yl)methoxy]-5-[2-([6-methoxy-5-[4-(oxetan-3-yl)piperazin-1-yl]pyridin-2-yl]amino)pyrimidin-4-yl]benzonitrile was obtained as an yellow solid (5 mg, 19% for 2 steps). HPLC: 99.8% purity, RT=3.73 min. MS: m/z=601.2 [M+H]HPLC: 96.0% purity, RT=3.23 min. MS: m/z=529.1 [M+H]⁺. ¹H NMR (300 MHz, DMSO-d₆, ppm) δ9.33 (s, 1H), 8.64-8.39 (m, 3 H), 7.77-7.68 (m, 1H), 7.54-7.41 (m, 2H), 7.31-7.22 (m, 1H), 4.64-4.30 (m, 6H), 4.00-3.92 (m, 1H), 3.88 (s, 3H), 3.74-3.40 (m, 4H), 3.00-2.94 (m, 5H), 2.43-2.37 (m, 4H).

2-([1-[(2S)-2-hydroxypropanoyl]azetidin-3-yl]methoxy)-5-[2-([6-methoxy-5-[4-(oxetan-3-yl)piperazin-1-yl]pyridin-2-yl]amino)pyrimidin-4-yl]benzonitrile: The title compound was prepared from 2-fluoro-5-(2-(6-methoxy-5-(4-(oxetan-3-yl)piperazin-1-yl)pyridin-2-ylamino)pyrimidin-4-yl)benzonitrile, tert-butyl 3-(hydroxymethyl)azetidine-1-carboxylate and (s)-2-hydroxypropanoic acid using Method E, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃), 20% to 50% gradient in 8 min; detector, UV 254 nm. 2-([1-[(2S)-2-hydroxypropanoyl]azetidin-3-yl]methoxy)-5-[2-([6-methoxy-5-[4-(oxetan-3-yl)piperazin-1-yl]pyridin-2-yl]amino)pyrimidin-4-yl]benzonitrile was obtained as an yellow solid (29 mg, 9% for 3 steps). HPLC: 99.8% purity, RT=3.73 min. MS: m/z=601.2 [M+H]⁺. ¹H NMR (300 MHz, DMSO-d₆, ppm) δ 9.33 (s, 1H), 8.60-8.45 (m, 3H), 7.72 (d, J=8.3 Hz, 1H), 7.55-7.42 (m, 2H), 7.26 (d, J=8.4 Hz, 1H), 5.03-4.93 (m, 1H), 4.60-4.33 (m, 7H), 4.20-4.07 (m, 2H), 4.07-3.94 (m, 1H), 3.89 (s, 3H), 3.78-3.68 (m, 1H), 3.51-3.41 (m, 1H), 3.14-3.08 (m, 1H), 3.00-2.94 (m, 4H), 2.43-2.37 (m, 4H), 1.17 (d, J=6.7, 1.9 Hz, 3H).

### Example 280: 2-([1-[(2R)-2-hydroxypropanoyl]azetidin-3-yl]methoxy)-5-[2-([6-methoxy-5-[4-(oxetan-3-yl)piperazin-1-yl]pyridin-2-yl]amino)pyrimidin-4-yl]benzonitrile

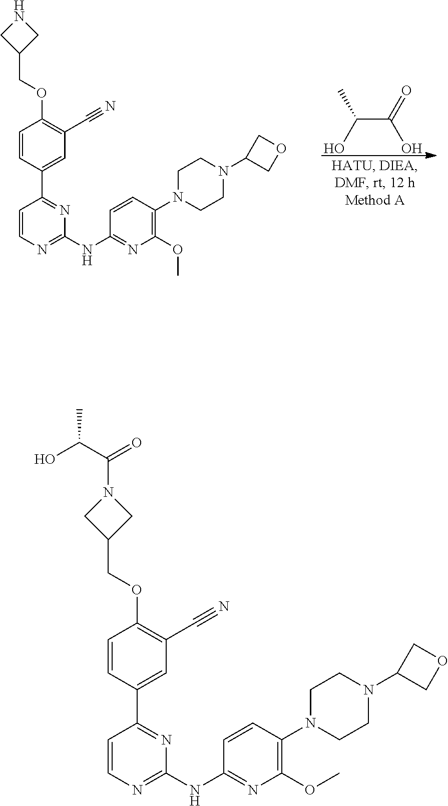

The title compound was prepared from 2-(azetidin-3-ylmethoxy)-5-(2-(6-methoxy-5-(4-(oxetan-3-yl)piperazin-1-yl)pyridin-2-ylamino)pyrimidin-4-yl)benzonitrile and (R)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₁HCO₃), 30% to 60% gradient in 8 min; detector, UV 254 nm. 2-([1-[(2R)-2-hydroxypropanoyl]azetidin-3-yl]methoxy)-5-[2-([6-methoxy-5-[4-(oxetan-3-yl)piperazin-1-yl]pyridin-2-yl]amino)pyrimidin-4-yl]benzonitrile was obtained as an yellow solid (18 mg, 25%). HPLC: 99.8% purity, RT=3.74 min. MS: m/z=601.2 [M+H]⁺. ¹H NMR (300 MHz, DMSO-d₆.ppm) δ 9.33 (s, 1H), 8.60-8.45 (m, 3H), 7.72 (d, J=8.3 Hz, 1H), 7.55-7.42 (m, 2H), 7.26 (d, J=8.4 Hz, 1H), 5.03-4.93 (m, 1H), 4.60-4.33 (m, 7H), 4.20-4.07 (m, 2H), 4.07-3.94 (m, 1H), 3.89 (s, 3H), 3.78-3.68 (m, 1H), 3.51-3.41 (m, 1H), 3.14-3.08 (m, 1H), 3.00-2.94 (m, 4H), 2.43-2.37 (m, 4H), 1.17 (d, J=6.7, 1.9 Hz, 3H).

### Example 281:2-([1-[(2S)-2-hydroxypropanoyl]pyrrolidin-3-yl]methoxy)-5-[2-([6-methoxy-5-[4-(oxetan-3-yl)piperazin-1-yl]pyridin-2-yl]amino)pyrimidin-4-yl]benzonitrile

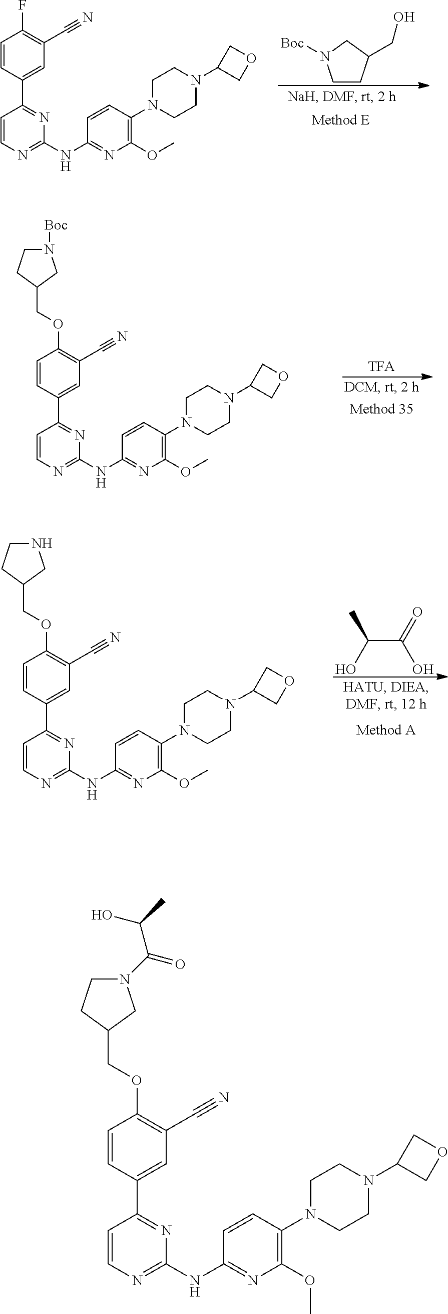

5-[2-([6-methoxy-5-[4-(oxetan-3-yl)piperazin-1-yl]pyridin-2-yl]amino)pyrimidin-4-yl]-2-[(pyrrolidin-3-yl)methoxy]benzonitrile: The title compound was prepared from 2-fluoro-5-(2-(6-methoxy-5-(4-(oxetan-3-yl)piperazin-1-yl)pyridin-2-ylamino)pyrimidin-4-yl)benzonitrile and tert-butyl 3-(hydroxymethyl)pyrrolidine-1-carboxylate using Method E and 35. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 25% to 45% gradient in 8 min; detector, UV 254 nm. 5-[2-([6-methoxy-5-[4-(oxetan-3-yl)piperazin-1-yl]pyridin-2-yl]amino)pyrimidin-4-yl]-2-[(pyrrolidin-3-yl)methoxy]benzonitrile was obtained as an yellow solid (8 mg, 29% for 2 steps). HPLC: 99.2% purity, RT=3.34 min. MS: m/z=543.3 [M+H]⁺. ¹H NMR (300 MHz, DMSO-d₆, ppm) δ9.36 (s, 1H), 8.59-8.52 (m, 2H), 8.52-8.44 (m, 1H), 7.77-7.68 (m, 1H), 7.54-7.47 (m, 1H), 7.47-7.38 (m, 1H), 7.31-7.22 (m, 1H), 4.60-4.50 (m, 2H), 4.50-4.40 (m, 2H), 4.30-4.07 (m, 2H), 3.88 (s, 3H), 3.52-3.40 (m, 1H), 3.05-2.62 (m, 8H), 2.42-2.36 (m, 4H), 2.11-1.81 (m, 2H), 1.48-1.40 (m, 1H).

2-([1-[(2S)-2-hydroxypropanoyl]pyrrolidin-3-yl]methoxy)-5-[2-([6-methoxy-5-[4-(oxetan-3-yl)piperazin-1-yl]pyridin-2-yl]amino)pyrimidin-4-yl]benzonitrile: The title compound was prepared from 2-fluoro-5-(2-(6-methoxy-5-(4-(oxetan-3-yl)piperazin-1-yl)pyridin-2-ylamino)pyrimidin-4-yl)benzonitrile, tert-butyl 3-(hydroxymethyl)pyrrolidine-1-carboxylate and (S)-2-hydroxypropanoic acid using Method E, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃), 40% to 70% gradient in 8 min; detector, UV 254 nm. 2-([1-[(2S)-2-hydroxypropanoyl]pyrrolidin-3-yl]methoxy)-5-[2-([6-methoxy-5-[4-(oxetan-3-yl)piperazin-1-yl]pyridin-2-yl]amino)pyrimidin-4-yl]benzonitrile was obtained as an yellow solid (30 mg, 18% for 3 steps). HPLC: 99.5% purity, RT=3.96 min. MS: m/z=615.3 [M+H]⁺. ¹H NMR (300 MHz, DMSO-d₆, ppm) δ 9.37 (s, 1H), 8.60-8.53 (m, 2H), 8.53-8.45 (m, 1H), 7.73 (d, J=8.3 Hz, 1H), 7.51 (d, J=5.3 Hz, 1H), 7.45 (d, J=9.2 Hz, 1H), 7.26 (d, J=8.3 Hz, 1H), 4.92-4.76 (m, 1H), 4.60-4.50 (m, 2H), 4.50-4.40 (m, 2H), 4.28-4.22 (m, 3H), 3.88 (s, 3H), 3.69-3.33 (m, 4H), 3.20-3.18 (m, 1H), 3.00-2.94 (m, 4H), 2.82-2.62 (m, 1H), 2.42-2.36 (m, 4H), 2.22-1.61 (m, 2H), 1.17 (d, 7=6.6 Hz, 3H).

### Example 282: 2-([1-[(2R)-2-hydroxypropanoyl]pyrrolidin-3-yl]methoxy)-5-[2-([6-methoxy-5-[4-(oxetan-3-yl)piperazin-1-yl]pyridin-2-yl]amino)pyrimidin-4-yl]benzonitrile

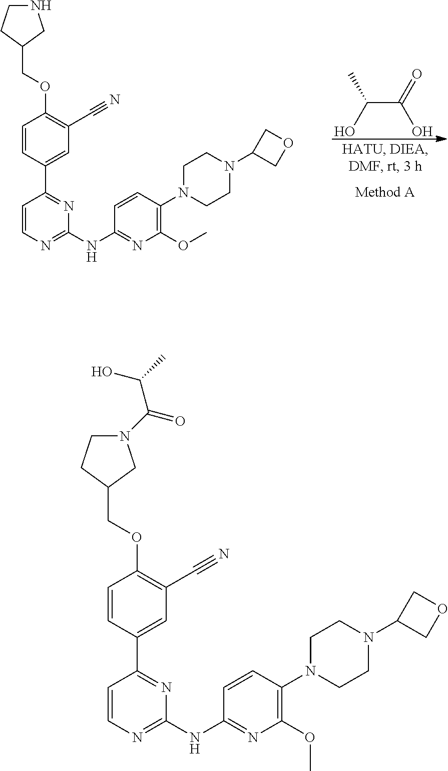

2-([1-[(2R)-2-hydroxypropanoyl]pyrolidin-3-yl]methoxy)-5-[2-([6-methoxy-5-[4-(oxetan-3-yl)piperazin-1-yl]pyridin-2-yl]amino)pyrimidin-4-yl]benzonitrile 2-([1-[(2R)-2-hydroxypropanoyl]pyrrolidin-3-yl]methoxy)-5-[2-([6-methoxy-5-[4-(oxetan-3-yl)piperazin-1-yl]pyridin-2-yl]amino)pyrimidin-4-yl]benzonitrile was prepared from 5-(2-(6-methoxy-5-(4-(oxetan-3-yl)piperazin-1-yl)pyridin-2-ylamino)pyrimidin-4-yl)-2-(pyrrolidin-3-ylmethoxy)benzonitrile and (R)-2-hydroxypropanoic acid using Method A. The final product was purified by prep-HPLC under the following conditions: column, XBridge Prep OBD C18 Column, 130×30 mm, 3 um; mobile phase, acetonitrile in water (with 10 mmol/L NH₄HCO₃), 40% to 70% gradient in 8 min; detector, UV 254 nm. 2-([1-[(2R)-2-hydroxypropanoyl]pyrrolidin-3-yl]methoxy)-5-[2-([6-methoxy-5-[4-(oxetan-3-yl)piperazin-1-yl]pyridin-2-yl]amino)pyrimidin-4-yl]benzonitrile was obtained as an yellow solid (28 mg, 45%). HPLC: 98.6% purity, RT=3.96 min. MS: m/z=615.2 [M+H]⁺. ¹H NMR (300 MHz, DMSO-d₆, ppm) δ 9.37 (s, 1H), 8.60-8.53 (m, 2H), 8.53-8.45 (m, 1H), 7.73 (d, J=8.3 Hz, 1H), 7.51 (d, J=5.3 Hz, 1H), 7.45 (d, J=9.2 Hz, 1H), 7.26 (d, J=8.3 Hz, 1H), 4.92-4.76 (m, 1H), 4.60-4.50 (m, 2H), 4.50-4.40 (m, 2H), 4.28-4.22 (m, 3H), 3.88 (s, 3H), 3.69-3.33 (m, 4H), 3.20-3.18 (m, 1H), 3.00-2.94 (m, 4H), 2.82-2.62 (m, 1H), 2.42-2.36 (m, 4H), 2.22-1.61 (m, 2H), 1.22-1.12 (m, 3H).

### Example 283: 2-([33-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)-5-methylpyrimidin-4-yl]benzonitrile

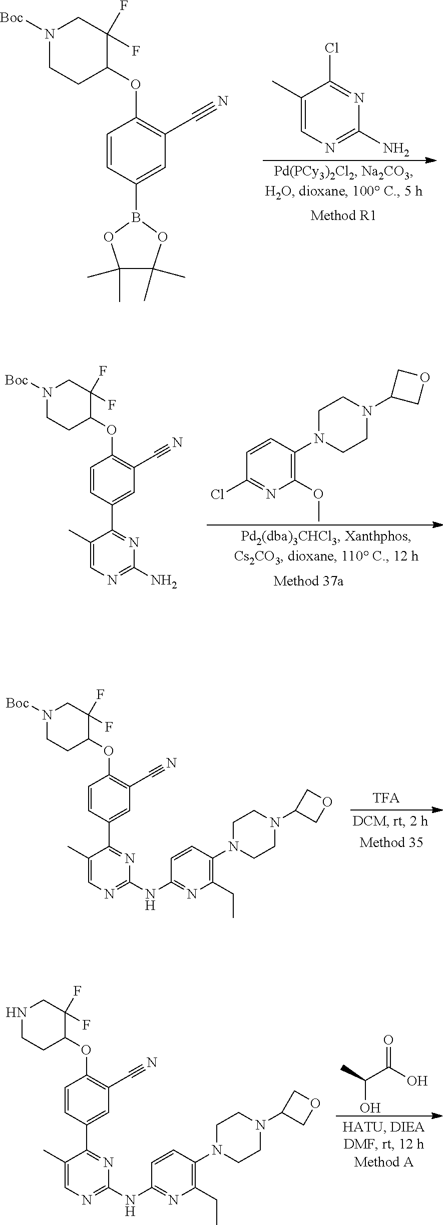

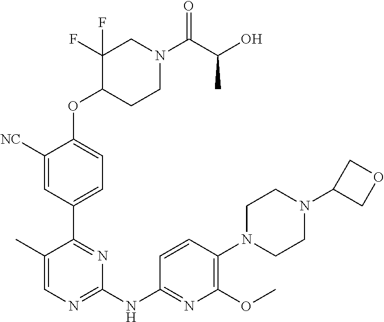 [SMILES: COc1nc(Nc2ncc(C)c(-c3ccc(OC4CCN(C(=O)[C@H](C)O)CC4(F)F)c(C#N)c3)n2)ccc1N1CCN(C2COC2)CC1]

2-[(3,3-difluoropiperidin-4-yl)oxy]-5-[2-([6-methoxy-5-[4-(oxetan-3-yl)piperazin-1-yl]pyridin-2-yl]amino)-5-methylpyrimidin-4-yl]benzonitrile: The title compound was prepared from tert-butyl 4-(2-cyano-4-(4,4,5,5-tetramethyl-1,3,2-dioxaborolan-2-yl)phenoxy)-3,3-difluoropiperidine-1-carboxylate, 4-chloro-5-methylpyrimidin-2-amine and 1-(6-chloro-2-methoxypyridin-3-yl)-4-(oxetan-3-yl)piperazine using Method R1, 37a and 35. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 28% to 51% gradient in 8 min; detector, UV 254 nm. 2-[(3,3-difluoropiperidin-4-yl)oxy]-5-[2-([6-methoxy-5-[4-(oxetan-3-yl)piperazin-1-yl]pyridin-2-yl]amino)-5-methylpyrimidin-4-yl]benzonitrile was obtained as a light yellow solid (3.4 mg, 5.2% for 3 steps). HPLC: 99.6% purity, RT=3.61 min. MS: m/z=593.1 [M+H]⁺. ¹H NMR (400 MHz, DMSO-d₆, ppm) δ9.20 (s, 1H), 8.45 (s, 1H), 8.14-8.09 (m, 1H), 8.07-7.99 (m, 1H), 7.72-7.65 (m, 1H), 7.63-7.56 (m, 1H), 7.26-7.19 (m, 1H), 5.20-5.16 (m, 1H), 4.55 (t, J=6.5 Hz, 2H), 4.46 (t, J=6.1 Hz, 2H), 3.87 (s, 3H), 3.51-3.42 (m, 1H), 3.31 (s, 2H), 3.19-3.15 (m, 1H), 3.05-2.80 (m, 6H), 2.73-2.69 (m, 1H), 2.42-2.37 (m, 4H), 2.26 (s, 3H), 2.14-1.68 (m, 2H).

2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)-5-methylpyrimidin-4-yl]benzonitrile: The title compound was prepared from 2-[(3,3-difluoropiperidin-4-yl)oxy]-5-[2-([6-methoxy-5-[4-(oxetan-3-yl)piperazin-1-yl]pyridin-2-yl]amino)-5-methylpyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 30% to 60% gradient in 8 min; detector, UV 254 nm. 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)-5-methylpyrimidin-4-yl]benzonitrile was obtained as a light yellow solid (33 mg, 36%). HPLC: 93.8% purity, RT=4.40 min. MS: m/z=665.2 [M+H]⁺. ¹H NMR (400 MHz, DMSO-d₆, ppm) δ 9.20 (s, 1H), 8.46 (s, 1H), 8.16-8.11 (m, 1H), 8.10-8.02 (m, 1H), 7.73-7.60 (m, 2H), 7.25-7.19 (m, 1H), 5.42-5.30 (m, 1H), 5.26-5.18 (m, 1H), 4.63-0.41 (m, 5H), 4.30-3.93 (m, 2H), 3.87 (s, 3H), 3.84-3.53 (m, 2H), 3.51-3.41 (m, 1H), 3.03-2.88 (m, 4H), 2.42-2.37 (m, 4H), 2.27 (s, 3H), 2.20-1.84 (m, 2H), 1.23 (d, J=6.5 Hz, 3H).

### Example 284: 2-[[(4S)-3,3-difluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)-5-methylpyrimidin-4-yl]benzonitrile and Example 285: 2-[[(4R)-3,3-difluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)-5-methylpyrimidin-4-yl]benzonitrile

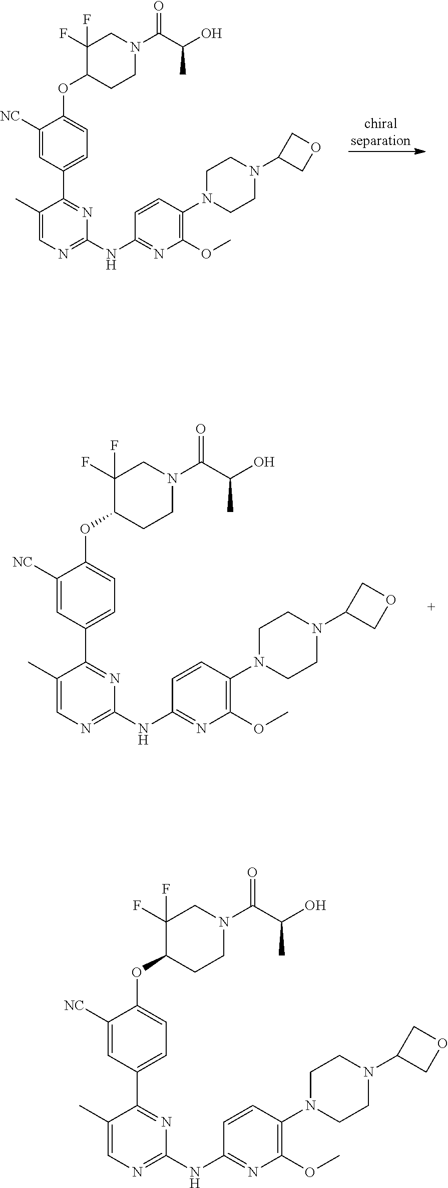 [SMILES: COc1nc(Nc2ncc(C)c(-c3ccc(OC4CCN(C(=O)[C@H](C)O)CC4(F)F)c(C#N)c3)n2)ccc1N1CCN(C2COC2)CC1 ; COc1nc(Nc2ncc(C)c(-c3ccc(O[C@H]4CCN(C(=O)[C@H](C)O)CC4(F)F)c(C#N)c3)n2)ccc1N1CCN(C2COC2)CC1 ; COc1nc(Nc2ncc(C)c(-c3ccc(O[C@@H]4CCN(C(=O)[C@H](C)O)CC4(F)F)c(C#N)c3)n2)ccc1N1CCN(C2COC2)CC1]

The two diastereomers were obtained by separation on chiral prep-HPLC under the following condition: column, CHIRALPAK IF-3, 0.46×5 cm, 3 um; mobile phase, (Hex: DCM=3:1)(0.1% DEA): MeOH=50:50, isocratic for 15 min; detector, UV 254 nm.

Example 284: (35 mg, 14%, light yellow solid) HPLC: 97.5% purity, RT=4.40 min. MS: m/z=665.2 [M+H]⁺. ¹H NMR (400 MHz, DMSO-d₆, ppm) δ 9.21 (s, 1H), 8.46 (s, 1H), 8.16-8.11 (m, 1H), 8.10-8.02 (m, 1H), 7.74-7.59 (m, 2H), 7.26-7.19 (m, 1H), 5.42-5.32 (m, 1H), 5.26-5.20 (m, 1H), 4.65-4.38 (m, 5H), 4.31-3.94 (m, 2H), 3.87 (s, 3H), 3.85-3.59 (m, 2H), 3.51-3.41 (m, 1H), 3.04-2.86 (m, 4H), 2.42-2.37 (m, 4H), 2.27 (s, 3H), 2.22-1.79 (m, 2H), 1.23 (d, J=6.4 Hz, 3H).

Example 285: (38 mg, 15%, light yellow solid) HPLC: 98.4% purity, RT=4.41 min. MS: m/z=665.2 [M+H]⁺. ¹H NMR (400 MHz, DMSO-d₆, ppm) δ9.23 (s, 1H), 8.46 (s, 1H), 8.18-8.11 (m, 1H), 8.11-8.02 (m, 1H), 7.74-7.60 (m, 2H), 7.27-7.18 (m, 1H), 5.40-5.34 (m, 1H), 5.27-5.19 (m, 1H), 4.61-4.41 (m, 5H), 4.35-3.94 (m, 2H), 3.88 (s, 3H), 3.85-3.53 (m, 2H), 3.51-3.41 (m, 1H), 2.99-2.93 (m, 4H), 2.44-2.37 (m, 4H), 2.27 (s, 3H), 2.23-1.82 (m, 2H), 1.24 (d, J=6.5 Hz, 3H).

### Example 286: 2-([33-difluoro-1-[(2R)-2-hydroxypropanoyl]piperidin-4-yl]oxy)-5-[2-([6-methoxy-5-[4-(oxetan-3-yl)piperazin-1-yl]pyridin-2-yl]amino)-5-methylpyrimidin-4-yl]benzonitrile

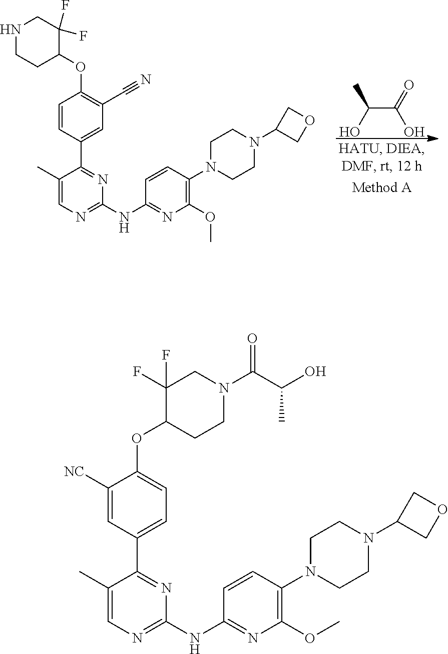

The title compound was prepared from 2-[(3,3-difluoropiperidin-4-yl)oxy]-5-[2-([6-methoxy-5-[4-(oxetan-3-yl)piperazin-1-yl]pyridin-2-yl]amino)-5-methylpyrimidin-4-yl]benzonitrile and (2R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 28% to 51% gradient in 8 min; detector, UV 254 nm. 2-([3,3-difluoro-1-[(2R)-2-hydroxypropanoyl]piperidin-4-yl]oxy)-5-[2-([6-methoxy-5-[4-(oxetan-3-yl)piperazin-1-yl]pyridin-2-yl]amino)-5-methylpyrimidin-4-yl]benzonitrile was obtained as a light yellow solid (25 mg, 20%). HPLC: 97.1% purity, RT=4.42 min. MS: m/z=665.2 [M+H]⁺. ¹H NMR (400 MHz, DMSO-d₆, ppm) δ 9.21 (s, 1H), 8.46 (s, 1H), 8.17-8.12 (m, 1H), 8.10-8.02 (m, 1H), 7.72-7.66 (m, 1H), 7.66-7.60 (m, 1H), 7.26-7.19 (m, 1H), 5.38-5.34 (m, 1H), 5.26-5.18 (m, 1H), 4.60-4.41 (m, 5H), 4.30-3.54 (m, 7H), 3.52-3.41 (m, 1H), 2.98-2.93 (m, 4H), 2.42-2.37 (m, 4H), 2.27 (s, 3H), 2.23-1.81 (m, 2H), 1.23 (d, J=6.5 Hz, 3H).

### Example 287: 2-([3,3-difluoro-1-[(2S)-2-hydroxypropanoyl]piperidin-4-yl]oxy)-5-[5-fluoro-2-([6-methoxy-5-[4-(oxetan-3-yl)piperazin-1-yl]pyridin-2-yl]amino)pyrimidin-4-yl]benzonitrile

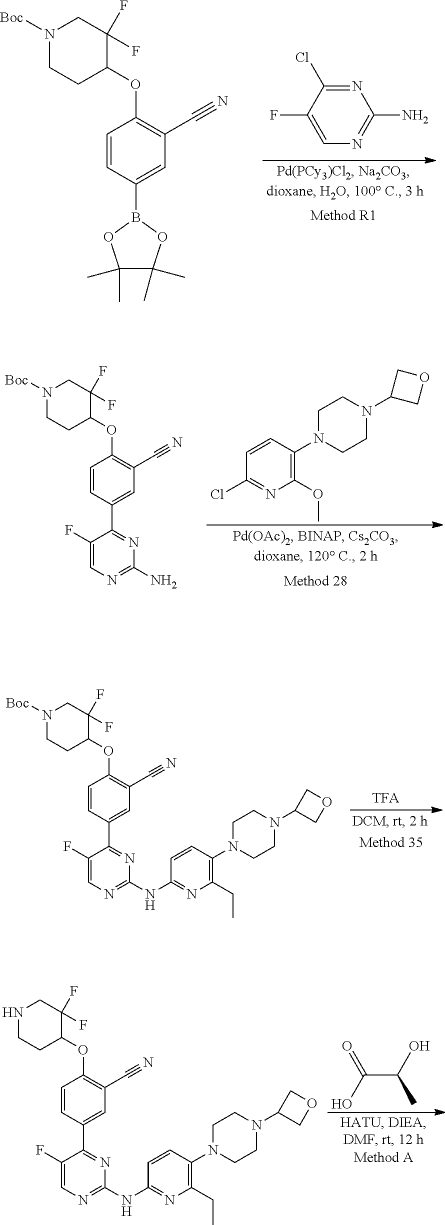

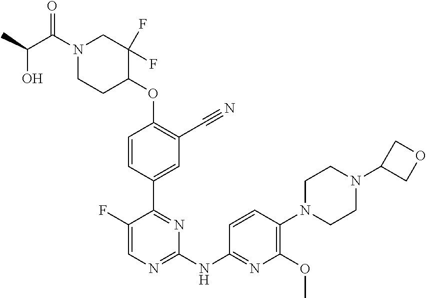 [SMILES: COc1nc(Nc2ncc(F)c(-c3ccc(OC4CCN(C(=O)[C@H](C)O)CC4(F)F)c(C#N)c3)n2)ccc1N1CCN(C2COC2)CC1]

The title compound was prepared from 1-tert-butyl 4-(2-cyano-4-(4,4,5,5-tetramethyl-1,3,2-dioxaborolan-2-yl)phenoxy)-3,3-difluoropiperidine-1-carboxylate, 4-chloro-5-fluoropyrimidin-2-amine, 1-(6-chloro-2-methoxypyridin-3-yl)-4-(oxetan-3-yl)piperazine and (S)-2-hydroxypropanoic acid using Method R1, 28,35 and A. The final product was purified by prep-HPLC under the following conditions: column, XBridge Prep OBD C18 Column, 150×19 mm, 5 um; mobile phase, EtOH in water (with 10 mmol/L NH₄HCO₃), 30% to 40% gradient in 8 min; detector, UV 254 nm. 2-([3,3-difluoro-1-[(2S)-2-hydroxypropanoyl]piperidin-4-yl]oxy)-5-[5-fluoro-2-([6-methoxy-5-[4-(oxetan-3-yl)piperazin-1-yl]pyridin-2-yl]amino)pyrimidin-4-yl]benzonitrile was obtained as an yellow solid (35 mg, 16% for 4 steps). HPLC: 97.1% purity, RT=7.72 min. MS: m/z=669.2 [M+H]⁺. ¹H NMR (300 MHz, DMSO-d₆, ppm) δ9.57 (s, 1H), 8.71-8.63 (m, 1H), 8.44-8.31 (m, 2H), 7.74-7.65 (m, 1H), 7.65-7.57 (m, 1H), 7.24 (d, J=8.3 Hz, 1H), 5.41-5.35 (m, 1H), 5.26-5.17 (m, 1H), 4.60-4.40 (m, 5H), 4.29-3.93 (m, 2H), 3.88 (s, 3H), 3.84-3.52 (m, 2H), 3.52-3.38 (m, 1H), 2.99-2.93 (m, 4H), 2.42-2.36 (m, 4H), 2.26-1.77 (m, 2H), 1.21 (d, J=6.5 Hz, 3H).

### Example 288: 2-[[(4S)-33-difluoro-1-[(2S)-2-hydroxypropanoyl]piperidin-4-yl]oxy]-5-[5-fluoro-2-([6-methoxy-5-[4-(oxetan-3-yl)piperazin-1-yl]pyridin-2-yl]amino)pyrimidin-4-yl]benzonitrile and Example 289: 2-[[(4R)-3,3-difluoro-1-[(2S)-2-hydroxypropanoyl]piperidin-4-yl]oxy]-5-[5-fluoro-2-([6-methoxy-5-[4-(oxetan-3-yl)piperazin-1-yl]pyridin-2-yl]amino)pyrimidin-4-yl]benzonitrile

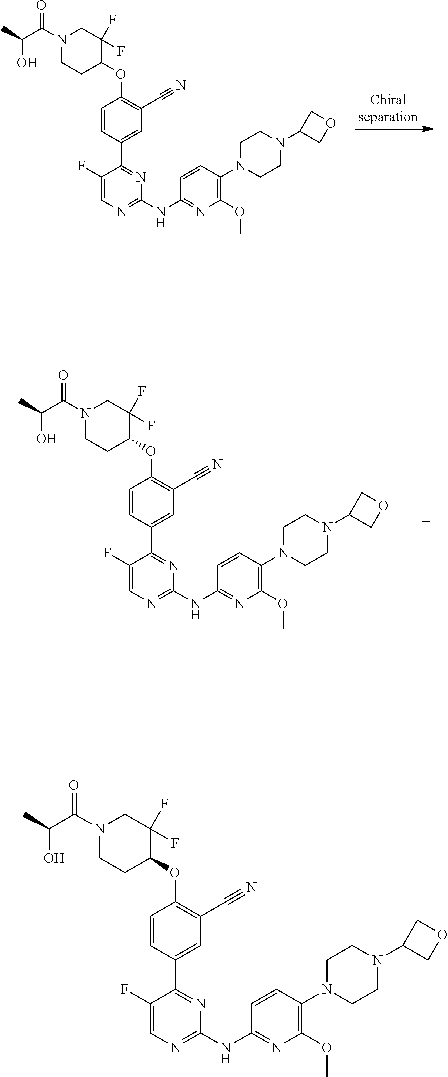 [SMILES: COc1nc(Nc2ncc(F)c(-c3ccc(OC4CCN(C(=O)[C@H](C)O)CC4(F)F)c(C#N)c3)n2)ccc1N1CCN(C2COC2)CC1 ; COc1nc(Nc2ncc(F)c(-c3ccc(O[C@@H]4CCN(C(=O)[C@H](C)O)CC4(F)F)c(C#N)c3)n2)ccc1N1CCN(C2COC2)CC1 ; COc1nc(Nc2ncc(F)c(-c3ccc(O[C@H]4CCN(C(=O)[C@H](C)O)CC4(F)F)c(C#N)c3)n2)ccc1N1CCN(C2COC2)CC1]

The two diastereomers were obtained by separation of 2-([3,3-difluoro-1-[(2S)-2-hydroxypropanoyl]piperidin-4-yl]oxy)-5-[5-fluoro-2-([6-methoxy-5-[4-(oxetan-3-yl)piperazin-1-yl]pyridin-2-yl]amino)pyrimidin-4-yl]benzonitrile on chiral prep-HPLC under the following conditions: column, Lux 3 um Cellulose-4, 0.46×15 cm, 3 um; mobile phase, IPA (with 0.1% DEA), 50% isocratic in 30 min; detector, UV 254 nm.

Example 288: (123 mg, 28%, yellow solid) HPLC: 99.7% purity, RT=4.90 min. MS: m/z=669.2 [M+H]⁺. ¹H NMR (300 MHz, DMSO-d₆, ppm) δ 9.57 (s, 1H), 8.71-8.63 (m, 1H), 8.44-8.31 (m, 2H), 7.74-7.65 (m, 1H), 7.65-7.57 (m, 1H), 7.24 (d, J=8.3 Hz, 1H), 5.41-5.35 (m, 1H), 5.26-5.17 (m, 1H), 4.60-4.40 (m, 5H), 4.29-3.93 (m, 2H), 3.88 (s, 3H), 3.84-3.52 (m, 2H), 3.52-3.38 (m, 1H), 2.99-2.93 (m, 4H), 2.42-2.36 (m, 4H), 2.26-1.77 (m, 2H), 1.21 (d, J=6.5 Hz, 3H).

Example 289: (118 mg, 27%, yellow solid) HPLC: 98.9% purity, RT=4.92 min. MS: m/z=669.2 [M+H]⁺. ¹H NMR (300 MHz, DMSO-d₆, ppm) δ9.58 (s, 1H), 8.70-8.63 (m, 1H), 8.44-8.31 (m, 2H), 7.74-7.65 (m, 1H), 7.65-7.57 (m, 1H), 7.24 (d, J=8.3 Hz, 1H), 5.42-5.34 (m, 1H), 5.26-5.18 (m, 1H), 4.60-4.32 (m, 5H), 4.28-3.92 (m, 2H), 3.87 (s, 3 H), 3.82-3.51 (m, 2H), 3.48-3.37 (m, 1H), 2.96 (s, 4H), 2.38 (s, 4H), 2.24-1.85 (m, 2H), 1.21 (d, 7=6.5 Hz, 3H).

### Example 290: 2-{[3,3-difluoro-1-(2-hydroxyacetyl)piperidin-4-yl]oxy}-5-[5-fluoro-2-({6-methoxy-5-[4-(oxetan-3-yl)piperazin-1-yl]pyridin-2-yl}amino)pyrimidin-4-yl]benzonitrile

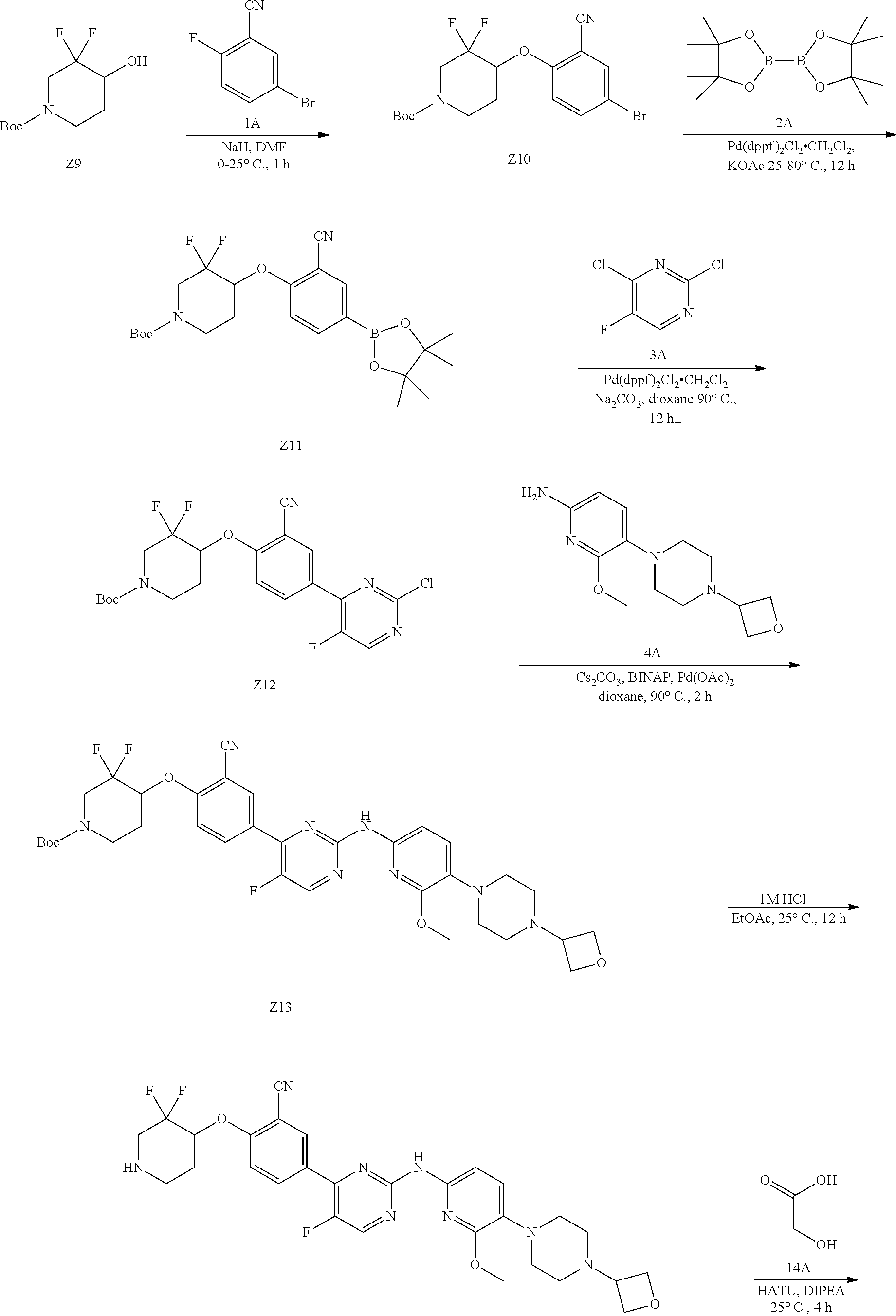

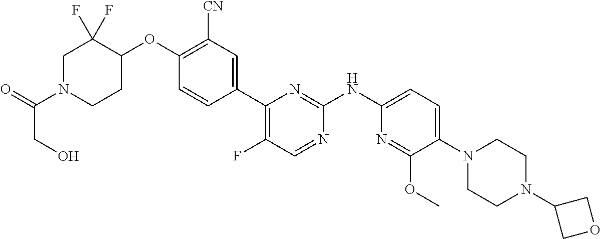 [SMILES: COc1nc(Nc2ncc(F)c(-c3ccc(OC4CCN(C(=O)CO)CC4(F)F)c(C#N)c3)n2)ccc1N1CCN(C2COC2)CC1]

Z10: tert-butyl 4-(4-bromo-2-cyanophenoxy)-3,3-difluoropiperidine-1-carboxylate: To a mixture of NaH (8.35 g, 208.0 mmol, 60.0% purity, 1.1 eq) in DMF (225 mL) was added a solution of compound Z9 (45.0 g, 190.0 mmol, 1 eq) in DMF (90 mL) at 0° C. and the mixture was stirred at 0° C. for 0.5 h. A solution of compound 1A (37.9 g, 190.0 mmol, 1 eq) in DMF (45 mL) was added dropwise and the mixture was stirred at 25° C. for 0.5 h. The reaction mixture was poured into aqueous saturated NH₄Cl (500 mL), extracted with ethyl acetate (800 mL×2). The organic phase was washed with water (300 mL×2), brine (300 mL), dried over sodium sulfate, filtered and concentrated under vacuum to give a crude product. The crude product was purified by silica gel chromatography (Petroleum ether/Ethyl acetate=10/l, 1/2) to afford compound Z10 (79.0 g, 177.0 mmol, 93.3% yield, 93.5% purity) as an yellow oil. LCMS: RT=0.994 min, MS[M+Na]*=439.0; ¹HNMR:, CDCl₃ 400 MHz. δ 7.69 (d, J=3.6 Hz, 1H), 7.65 (dd, J=3.6, 8.8 Hz, 1H), 6.99 (d, J=8.8 Hz, 1H), 4.65 (dd, J=3.2, 6.4 Hz, 1H), 4.38-4.13 (m, 1H), 4.05-3.84 (m, 1H), 3.76-3.51 (m, 1H), 3.48-3.22 (m, 1H), 2.14-2.05 (m, 2H), 1.48 (s, 9H).

Z11: tert-butyl 4-[2-cyano-4-(4,4,5,5-tetramethyl-1,3,2-dioxaborolan-2-yl)phenoxy]-3,3-difluoropiperidine-1-carboxylate: To a mixture of compound Z10 (25.0 g, 59.9 mmol, 1 eq), compound 2A (16.7 g, 65.9 mmol, 1.1 eq), KOAc (17.6 g, 180.0 mmol, 3 eq) in 1,4-dioxane (125 mL) was added Pd(dppf)Cl₂ CH₂Cl₂ (2.45 g, 3.00 mmol, 0.05 eq) at 25° C. and the mixture was heated to 80° C. for 12 h under nitrogen atmosphere. The reaction mixture was filtered, washed with ethyl acetate (400 mL) and the filtrate was diluted with water (400 mL). The phases were separated and the aqueous layer was extracted with ethyl acetate (400 mL). The organic phases were combined, washed with water (200 mL×2) and brine (200 mL). Dried over sodium sulfate and concentrated under vacuum to give compound Z11 (32 g, crude) as black gum which was used directly without purification. LCMS: RT=1.009 min, MS: [M+Na]⁺, 487.1 ¹HNMR: CDCl₃ 400 MHz. δ 8.04 (d, J=1.2 Hz, 1H), 7.96 (dd, J=1.6, 8.8 Hz, 1H), 7.05 (d, J=8.0 Hz, 1H), 4.75 (m, 1H), 4.35-3.87 (m, 2H), 3.68-3.18 (m, 2H), 2.07 (s, 2H), 1.48 (s, 9H), 1.34 (s, 12H).

Z12: tert-butyl 4-[4-(2-chloro-5-fluoropyrimidin-4-yl)-2-cyanophenoxy]-3,3-difluoropiperidine-1-carboxylate: To a solution of compound Z11 (32.0 g, 68.9 mmol, 1 eq) and compound 3A (11.5 g, 68.9 mmol, 1 eq) in 1,4-dioxane (160 mL) were added Pd(dppf)Cl₂.CH₂Cl₂ (2.81 g, 3.45 mmol, 0.05 eq) and Na₂CO₃ (11.0 g, 103.4 mmol, 1.5 eq). The mixture was stirred at 90° C. for 12 h. The mixture was concentrated under vacuum to give a residue. The residue was purified by flash silica gel chromatography (petroleum ether/ethyl acetate=10/1-5/1) to give compound Z12 (22.0 g, 38.5 mmol, 55.9%, 82.1% purity) as an yellow oil. LCMS: RT=0.959 min, MS: [M+Na]⁺, 491.0. ¹H NMR: (CDCl₃, 400 MHz) δ 8.57 (d, J=3.2 Hz, 1H), 8.47 (d, J=3.6 Hz, 1H), 8.42 (dd, J=2.4, 9.2 Hz, 1H), 7.24 (br d, J=9.2 Hz, 1H), 4.83 (br s, 1H), 4.51-4.19 (m, 1H), 4.03 (br s, 1H), 3.81-3.13 (m, 2H), 2.23-2.07 (m, 2H), 1.50-1.49 (m, 9H).

Z13: tert-butyl 4-{2-cyano-4-[5-fluoro-2-({6-methoxy-5-[4-(oxetan-3-yl)piperazin-1-yl]pyridin-2-yl}amino)pyrimidin-4-yl]phenoxy}-3,3-difluoropiperidine-1-carboxylate: A mixture of compound Z12 (1.60 g, 3.40 mmol, 1 eq), compound 4A (900.0 mg, 3.40 mmol, 1 eq), Cs2CO3 (2.22 g, 6.81 mmol, 2 eq), BINAP (424.0 mg, 681.0 umol, 0.2 eq) and Pd(OAc)₂ (152.9 mg, 681.0 umol, 0.2 eq) in dioxane (20 mL) was degassed and purged with N₂ for 3 times, and then the mixture was stirred at 90° C. for 2 hr under N₂ atmosphere. Water (40 mL) was poured into reaction mixture. The aqueous phase was extracted with ethyl acetate/ethanol (v/v=10/1,100 mL×2). The combined organic phase was washed with brine (20 mL), dried with anhydrous Na₂SO₄, filtered and concentrated in vacuum to give a crude product. The crude compound Z13 (2.30 g, crude) was used directly without purification confirmed. LCMS: RT=0.888 min, MS: [M+1]⁺, 697.2.

2-[(3,3-difluoropiperidin-4-yl)oxy]-5-[5-fluoro-2-({6-methoxy-5-[4-(oxetan-3-yl)piperazin-1-yl]pyridin-2-yl}amino)pyrimidin-4-yl]benzonitrile: To a solution of compound Z13 (2.30 g, 3.30 mmol, 1 eq) in EtOAc (25 mL) was added aqueous HCl (1 M, 75 mL, 22.7 eq). The mixture was stirred for 12 h at 25° C. The mixture was adjusted with Na₂CO₃ to pH˜8. The mixture was extracted with ethyl acetate (100 mL×2). The combined organic phase was washed, with brine (30 mL), dried with anhydrous Na₂SO₄, filtered and concentrated in vacuum to give a crude product. The crude was purified by silica gel chromatography (Petroleum ether/Ethyl acetate=1/1˜ Ethyl acetate/Ethanol=5/1) to afford the title compound (743.0 mg, 1.18 mmol, 35.9% yield, 95.1% purity) as an yellow solid. LCMS: RT=0.936 min, MS: [M+1]⁺, 597.3 ¹HNMR:, (CDCl₃ 400 MHz) δ 8.40-8.44 (m, 2H), 8.37 (dd, J=2.4, 9.2 Hz, 1H), 7.78 (d, J=8.4 Hz, 1H), 7.64 (s, 1H), 7.26-7.20 (m, 2H), 4.81 (m, 1H), 4.70-4.73 (m, 4H), 3.97 (s, 3H), 3.61 (t, J=6.4 Hz, 1H), 3.51-3.34 (m, 1H), 3.24-3.03 (m, 6H), 2.92 (d, J=14.0 Hz, 1H), 2.57 (s, 4H), 2.20-2.09 (m, 2H).

2-{[3,3-difluoro-1-(2-hydroxyacetyl)piperidin-4-yl]oxy}-5-[5-fluoro-2-({6-methoxy-5-[4-(oxetan-3-yl)piperazin-1-yl]pyridin-2-yl}amino)pyrimidin-4-yl]benzonitrile: A mixture of 2-[(3,3-difluoropiperidin-4-yl)oxy]-5-[5-fluoro-2-({6-methoxy-5-[4-(oxetan-3-yl)piperazin-1-yl]pyridin-2-yl}amino)pyrimidin-4-yl]benzonitrile (200.0 mg, 335.0 umol, 1 eq), compound 14A (30.6 mg, 402.0 umol, 24.5 uL, 1.2 eq), HATU (140.0 mg, 369.0 umol, 1.1 eq) and DIPEA (65.0 mg, 503.0 umol, 87.6 uL, 1.5 eq) in DMF (5 mL) was stirred for 4 h at 25° C. The residue was poured into water (20 mL). The aqueous phase was extracted with ethyl acetate (30 mL×3). The combined organic phase was washed with brine (10 mL), dried with anhydrous Na₂SO₄, filtered and concentrated in vacuum to give a crude product. The crude product was purified by pre-HPLC (column: Phenomenex Gemini 150×25 mm×10 um; mobile phase: [water(0.04% NH₃H₂CM-10 mM NH₄HCO₃)-ACN]; B %: 30%-60%, 10 min) and lyophilized to give the title compound (64.8 mg, 98.9 umol, 29.5% yield, 100% purity) as an yellow solid LCMS: RT=0.915 min, MS: [M+1]⁺, 655.4; HPLC: RT=1.815 min, 100% purity; ¹HNMR: (CDCl₃, 400 MHz) δ 8.48-8.36 (m, 3H), 7.76 (d, 7=8.0 Hz, 1H), 7.67 (s, 1H), 7.22-7.27 (m, 1H), 4.92-4.49 (m, 6H), 4.35-4.18 (m, 2H), 3.97 (s, 3H), 3.92-3.28 (m, 5H), 3.12 (s, 4H), 2.57 (s, 4H), 2.30-2.09 (m, 2H).

### Example 291: 2-{[33-difluoro-1-(2-hydroxypropanoyl)piperidin-4-yl]oxy}-5-[5-fluoro-2-({6-methoxy-5-[4-(oxetan-3-yl)piperazin-1-yl]pyridin-2-yl}amino)pyrimidin-4-yl]

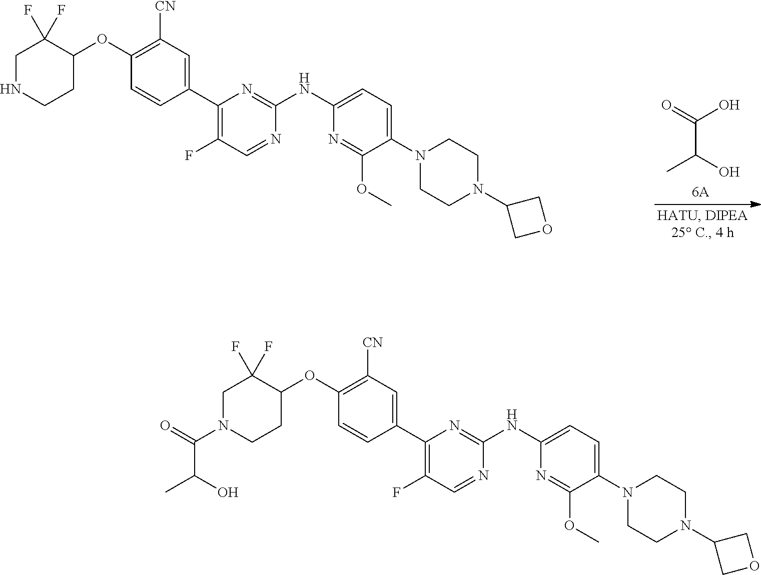

A mixture of 2-[(3,3-difluoropiperidin-4-yl)oxy]-5-[5-fluoro-2-({6-methoxy-5-[4-(oxetan-3-yl)piperazin-1-yl]pyridin-2-yl}amino)pyrimidin-4-yl]benzonitrile (200.0 mg, 335.0 umol, 1 eq), 2-hydroxyacetic acid, 6A (36.2 mg, 402.0 umol, 30.0 uL, 1.2 eq), HATU (140.0 mg, 369.0 umol, 1.1 eq) and DIPEA (65.0 mg, 503.0 umol, 87.6 uL, 1.5 eq) in DMF (5 mL) was stirred for 4 h at 25° C. The residue was poured into water (20 mL). The aqueous phase was extracted with ethyl acetate (30 mL×3). The combined organic phase was washed with brine (10 mL), dried with anhydrous Na₂SO₄, filtered and concentrated in vacuum to give a crude product. The crude product was purified by pre-HPLC (column: Phenomenex Syneigi C18 150×25×10 um; mobile phase: [water(0.225% FA)-ACN]; B %: 19%-37%, 9 min) and lyophilized to obtain the title compound (70.9 mg, 106.0 umol, 31.5% yield, 99.7% purity) as an yellow solid. LCMS: RT=0.931 min, MS: [M+1]⁺, 669.4; HPLC: RT=1.918 min, 99.7% purity. ¹HNMR: (CDCl₃, 400 MHz), δ 8.50-8.36 (m, 3H), 8.11 (s, 2H), 7.85-7.69 (m, 2H), 7.26-7.20 (m, 1H), 4.90 (s, 1H), 4.88-4.81 (m, 2H), 4.79-4.71 (m, 2H), 4.61-4.49 (m, 1H), 3.98 (s, 3H), 3.95 (s, 6H), 3.82-3.76 (m, 1H), 3.71 (s, 1H), 3.63-3.30 (m, 1H), 3.28-3.05 (m, 4H), 2.80 (s, 4H), 2.31-2.08 (m, 2H), 1.52-1.34 (m, 3H).

### Example 292: 2-({3,3-difluoro-1-[(2S)-2-hydroxypropanoyl]piperidin-4-yl}oxy)-5-[5-fluoro-2-({6-methoxy-5-[4-(oxetan-3-yl)piperazin-1-yl]pyridin-2-yl}amino)pyrimidin-4-yl]benzonitrile

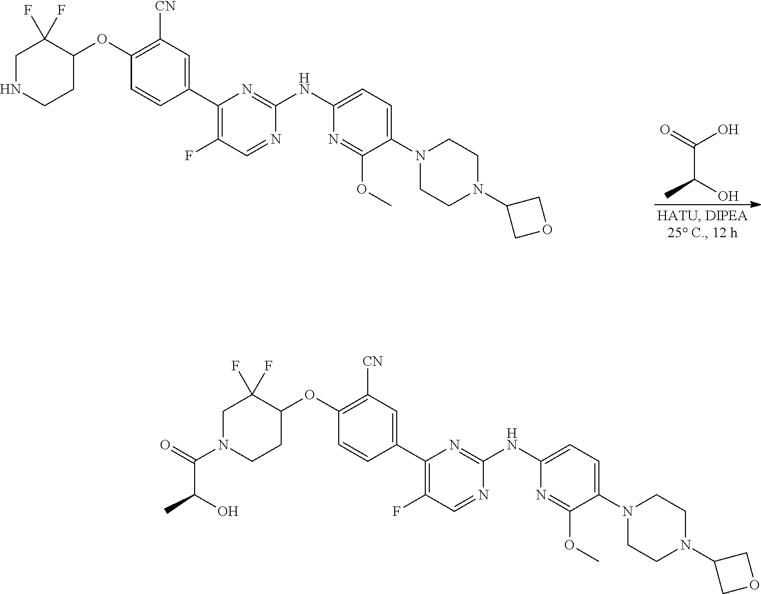

A mixture of 2-[(3,3-difluoropiperidin-4-yl)oxy]-5-[5-fluoro-2-({6-methoxy-5-[4-(oxetan-3-yl)piperazin-1-yl]pyridin-2-yl}amino)pyrimidin-4-yl]benzonitrile (200.0 mg, 335.0 umol, 1 eq), (2S)-2-hydroxypropanoic acid (30.2 mg, 335.0 umol, 25.0 uL, 1 eq), HATU (140.0 mg, 369.0 umol, 1.1 eq) and DIPEA (65.0 mg, 503.0 umol, 87.6 uL, 1.5 eq) in DMF (5 mL) was stirred for 12 h at 25° C. The residue was poured into water (20 mL). The aqueous phase was extracted with ethyl acetate (30 mL×3). The combined organic phase was washed with brine (10 mL), dried with anhydrous Na₂SO₄, filtered and concentrated in vacuum to give a crude product. The crude product was purified by pre-HPLC (column: Phenomenex Synergi C18 150×25×10 um; mobile phase: [water(0.225% FA)-ACN]; B %: 25%-55%, 10 min) and lyophilized to give the title compound 7 (35.11 mg, 52.3 umol, 15.6% yield, 99.5% purity) as an yellow solid. LCMS: RT=0.732 min, MS: [M+1]⁺, 669.4; HPLC: RT=1.552 min, 99.5% purity. ¹HNMR: (CDCl₃, 400 MHz) δ 8.47-8.38 (m, 3H), 8.10 (s, 2H), 7.80-7.73 (m, 2H), 7.25 (s, 1H), 4.90 (s, 1H), 4.84-4.71 (m, 4H), 4.61-4.49 (m, 1H), 3.98 (s, 4H), 3.80-3.29 (m, 3H), 3.25-3.17 (m, 4H), 3.12-3.03 (m, 4H), 2.74 (s, 4H), 2.21 (m, 2H), 1.51-1.34 (m, 3H).

### Example 293: 2-({3,3-difluoro-1-[(2S)-2-hydroxypropanoyl]piperidin-4-yl}oxy)-5-[5-fluoro-2-({4-[1-(oxetan-3-yl)piperidin-4-yl]phenyl}amino)pyrimidin-4-yl]benzonitrile

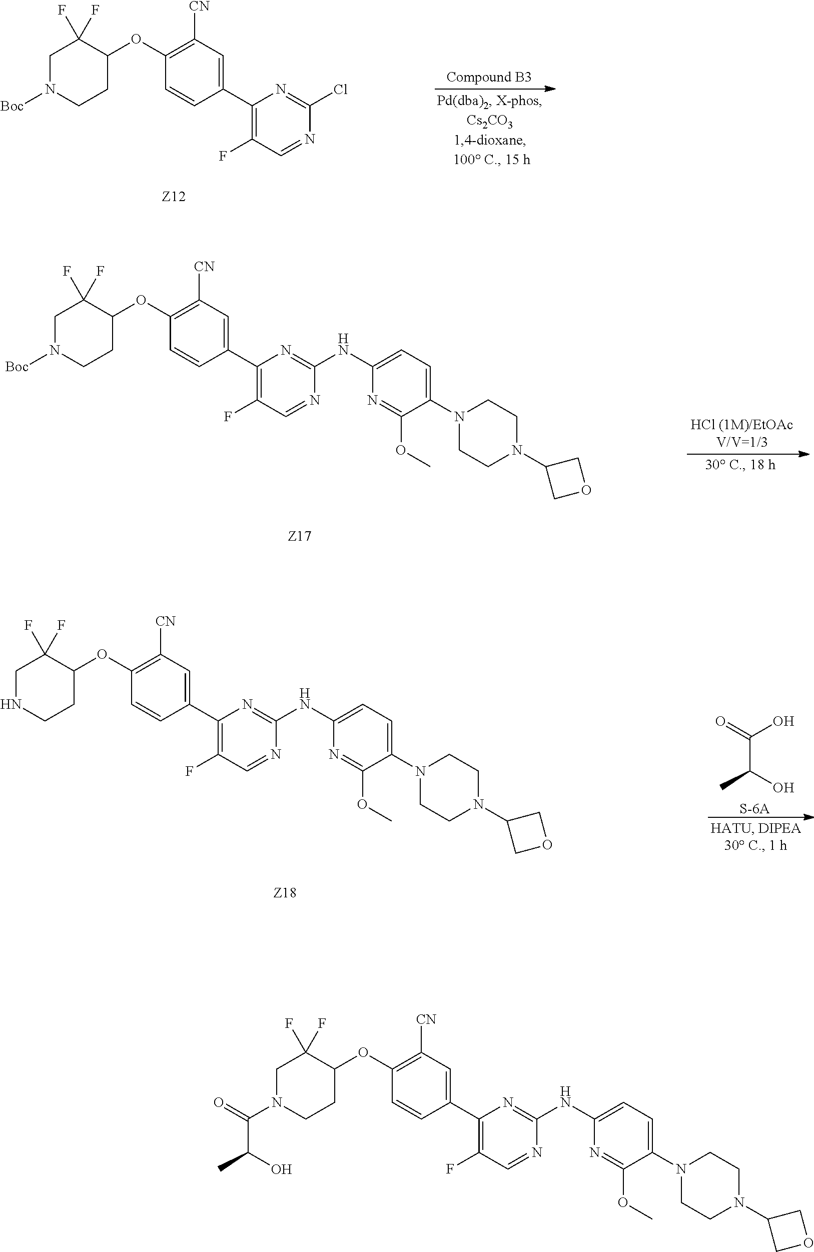

Z17: tert-butyl 4-{2-cyano-4-[5-fluoro-2-({6-methoxy-5-[1-(oxetan-3-yl)piperidin-4-yl]pyridin-2-yl}amino)pyrimidin-4-yl]phenoxy}-33-difluoropiperidine-1-carboxylate: To a solution of compound Z12 (500.0 mg, 1.07 mmol, 1.00 eq) in dioxane (10 mL) was added compound B3 (351.0 mg, 1.33 mmol, 1.25 eq), X-phos (152.0 mg, 320.0 umol, 0.300 eq), Cs₂CO₃ (695.0 mg, 2.13 mmol, 2.00 eq) and Pd(dba)₂ (293.0 mg, 320.0 umol, 0.300 eq). The mixture was stirred at 100° C. for 1.5 h. The reaction mixture was diluted with H₂O (50 mL) and extracted with EtOAc 180 mL (60 mL×3). The combined organic layers were washed with brine (80 mL), dried over Na₂SO₄. filtered and concentrated under reduced pressure to give a red solid. The compound Z17 (1.40 g, crude) was obtained as a red solid which was used in the next step directly. LCMS: RT=0.948 min, m/z (M+H⁺)=696.4.

Z18:2-[(3,3-difluoropiperidin-4-yl)oxy]-5-[5-fluoro-2-({6-methoxy-5-[1-(oxetan-3-yl)piperidin-4-yl]pyridin-2-yl}amino)pyrimidin-4-yl]benzonitrile: To a solution of compound Z17 (1.43 g, 2.06 mmol, 1.00 eq) in EtOAc (80 mL) was added HCl (12.0 M, 20.8 mL, 121.6 eq) and H₂O (150 mL). The mixture was stirred at 30° C. for 18 h. the aqueous phase was neutralized with sat.Na₂CO₃ to obtain The title compound as a white solid (1.00 g, crude) which was used in the next step directly. LCMS: RT=0.748 min, m/z (M+H⁺)=596.2.

2-({3,3-difluoro-1-[(2S)-2-hydroxypropanoyl]piperidin-4-yl}oxy)-5-[5-fluoro-2-({4-[1-(oxetan-3-yl)piperidin-4-yl]phenyl}amino)pyrimidin-4-yl]benzonitrile: The title compound was prepared from 2-[(3,3-difluoropiperidin-4-yl)oxy]-5-[5-fluoro-2-({6-methoxy-5-[1-(oxetan-3-yl)piperidin-4-yl]pyridin-2-yl}amino)pyrimidin-4-yl]benzonitrile (500.0 mg, 840.0 umol, 1.00 eq) and (S)-2-hydroxypropanoic acid (152.0 mg, 1.68 mmol, 125.0 uL, 2.00 eq) using Method A. The product was purified by prep-HPLC (column: Luna C18 150×25×5u; mobile phase: [water (0.225% FA)−ACN]; B %: 20%-50%, 10 min) to obtain The title compound (47.2 mg, 7.43%) as a yellow solid. LCMS: RT=0.995 min, m/z (M+H⁺)=668.3 HPLC: RT=5.84 min, ¹HNMR: (400MHZ, CDCl₃) δ 8.32-8.38 (m, 3H), 7.69-7.71 (s, 1H), 7.64 (m, 1H), 7.42-7.44 (m, 1H), 7.15-7.22 (m, 1H), 4.64 (s, 2H), 4.62-4.63 (m, 4H), 4.44 (dd, 7=6.90, 13.44 Hz, 2H), 3.84 (s, 4H), 3.50-3.62 (m, 3H), 3.48 (d, 7=10.04 Hz, 1H), 2.84 (d, 7=10.8 Hz, 2H), 2.78 (s, 1H), 1.92 (d, 7=12.80 Hz, 2H), 1.82 (m, 2H), 1.78-1.79 (s, 3H), 1.30-1.33 (m, 3H).

### Example 294: 2-{[33-difluoro-1-(2-hydroxypropanoyl)piperidin-4-yl]oxy}-5-[5-fluoro-2-({6-methoxy-5-[1-(oxetan-3-yl)piperidin-4-yl]pyridin-2-yl}amino)pyrimidin-4-yl]benzonitrile

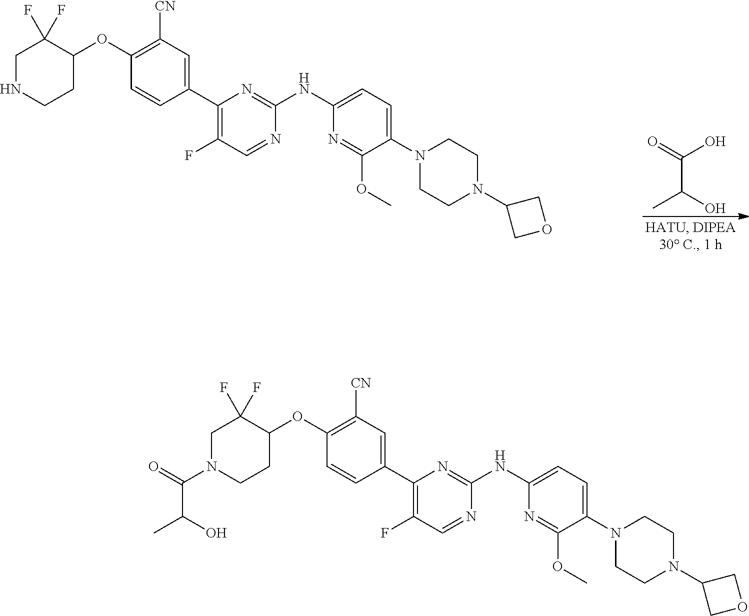

The title compound was prepared from 2-[(3,3-difluoropiperidin-4-yl)oxy]-5-[5-fluoro-2-({6-methoxy-5-[1-(oxetan-3-yl)piperidin-4-yl]pyridin-2-yl}amino)pyrimidin-4-yl]benzonitrile (500.0 mg, 840.0 umol, 1.00 eq) and 2-hydroxypropanoic acid (151.0 mg, 1.68 mmol, 125.0 uL, 2.00 eq) using Method A. The product was purified by prep-HPLC to obtain the title compound (60.9 mg, 9.89%) was obtained as red oil. LCMS: RT=1.00 min, m/z (M+H⁺)=668.4. HPLC: RT=2.79 min, 97.2%. ¹HNMR: (400MHZ, CDCl₃) δ 8.41-8.45 (m, 3H), 7.77 (s, 1H), 7.73 (m, 1H), 7.48-7.52 (m, 1H), 7.30 (m, 1H), 4.75 (s, 2H), 4.69-4.72 (m, 4H), 4.52 (dd, 7=6.90, 13.44 Hz, 2H), 3.92 (s, 4H), 3.61-3.70 (m, 3H), 3.31 (d, 7=10.04 Hz, 1H), 2.98 (d, 7=10.8 Hz, 2H), 2.84 (s, 1H), 2.23 (d, 7=12.80 Hz, 2H), 2.19 (m, 2H), 1.88 (s, 3H), 1.38-1.45 (m, 3H).

### Example 295: 2-{[(3R,4S)-3-fluoro-1-(2-hydroxypropanoyl)piperidin-4-yl]oxy}-5-[5-fluoro-2-({4-[4-(oxetan-3-yl)piperazin-1-yl]phenyl}amino)pyrimidin-4-yl]benzonitrile

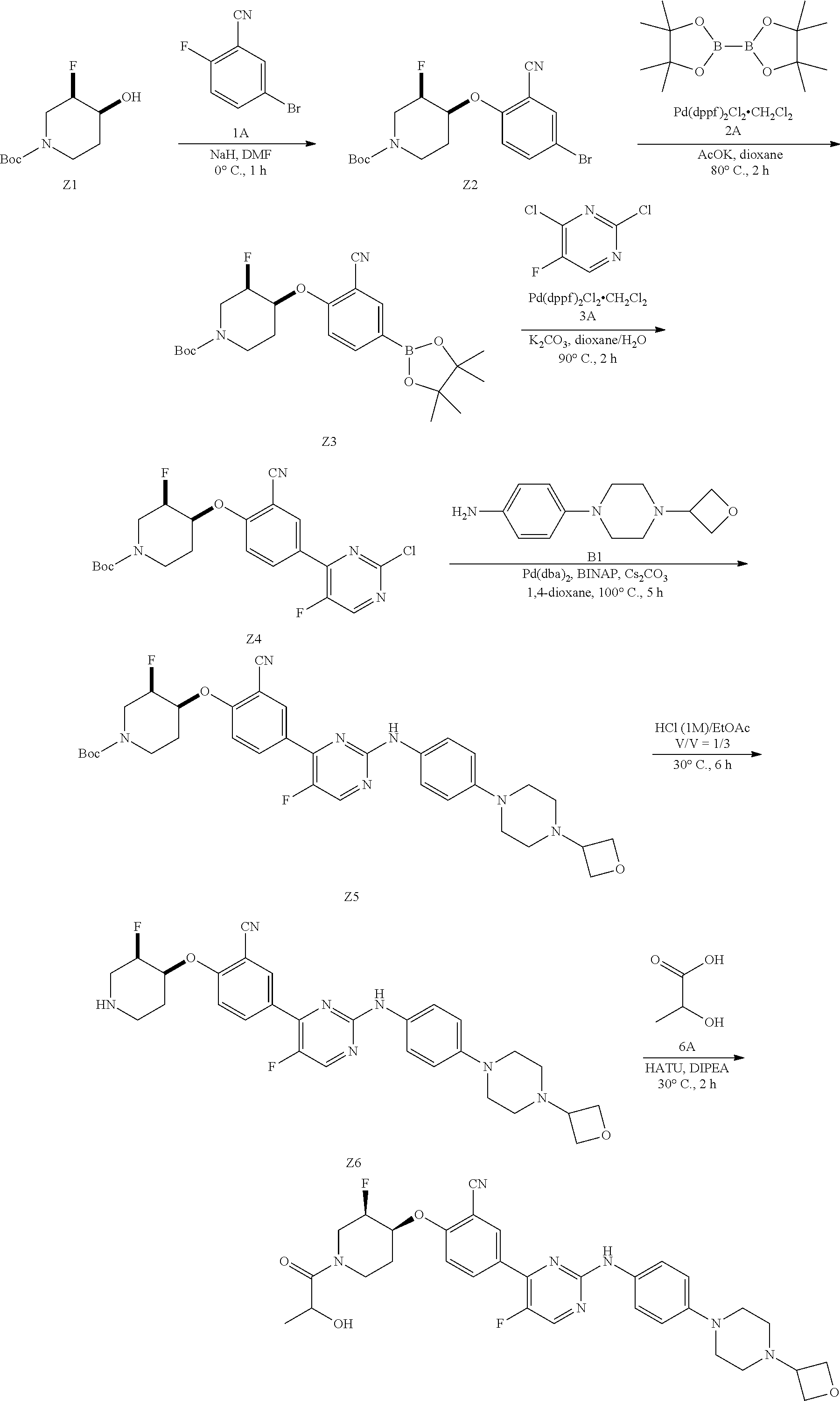

Z2: tert-butyl 4-(4-bromo-2-cyanophenoxy)-3-fluoropiperidine-1-carboxylate: To a mixture of NaH (1.75 g, 43.7 mmol, 60% purity, 1.10 eq) in DMF (150 mL) was added a solution of Z1 (8.70 g, 39.7 mmol, 1.00 eq) in DMF (10 mL) at 0° C. The mixture was then stirred at 0° C. for 0.5 h. Then a solution of 1A (7.94 g, 39.7 mmol, 1.00 eq) in DMF (10 mL) was added to the mixture and the mixture was then stirred at 0° C. for another 0.5 h. The mixture was quenched with saturated NH₄Cl solution (600 mL) and then extracted with EtOAc (300 mL×3). The combined organic phase was washed with water (300 mL×2), dried with anhydrous Na₂SO₄, filtered and concentrated to give tert-butyl 4-(4-bromo-2-cyanophenoxy)-3-fluoropiperidine-1-carboxylate, Z2 (17.0 g, crude) as an yellow oil which was used in the next step directly. ¹HNMR: (CDCl₃, 400MHZ) δ 7.61 (d, J=2.4 Hz, 1H), 7.56 (dd, J=2.8, 9.2 Hz, 1H), 6.91 (d, J=8.8 Hz, 1H), 4.71-4.67 (m, 2H), 3.60-3.57 (m, 1H), 3.45-3.39 (m, 1H), 2.05-2.00 (m, 1H), 1.79-1.74 (m, 1H), 1.40 (s, 9H), 0.81-0.76 (m, 2H). LCMS: RT=1.52 min, m/z (M-56+H⁺)=342.8.

Z3: tert-butyl 4-[2-cyano-4-(4,4,5,5-tetramethyl-1,3,2-dioxaborolan-2-yl)phenoxy]-3-fluoropiperidine-1-carboxylate: To a mixture of Z2 (17.0 g, 42.6 mmol, 1.00 eq), 2A (11.9 g, 46.8 mmol, 1.10 eq) and AcOK (8.36 g, 85.2 mmol, 2.00 eq) in dioxane (100 mL) was added Pd(dppf)Cl₂.CH₂Cl₂ (1.74 g, 2.13 mmol, 0.05 eq) under N2. The mixture was then stirred at 80° C. for 2h. The mixture was concentrated to remove dioxane and then diluted with water (500 mL) and EtOAc (500 mL). The aqueous phase was extracted with EtOAc (500 mL×3), dried with anhydrous Na₂SO₄, filtered and concentrated to give tert-butyl 4-[2-cyano-4-(4,4,5,5-tetramethyl-1,3,2-dioxaborolan-2-yl)phenoxy]-3-fluoropiperidine-1-carboxylate (19.0 g, crude) as a crude brown oil which was used in the next step directly. LCMS: RT=1.058 min, m/z (M-56+H⁺)=391.3; ¹HNMR: EW8546-5-P1A1 (CDCl₃, 400 MHz) δ 8.05 (d, J=1.6 Hz, 1H), 7.96 (dd, J=2.4, 8.4 Hz, 1H), 7.06 (d, J=8.4 Hz, 1H), 4.90-4.85 (m, 2H), 3.96-3.77 (m, 2H), 3.56-3.53 (m, 2H), 2.16-2.12 (m, 1H), 1.87-1.82 (m, 1H), 1.49 (s, 9H), 1.35 (s, 12H), 0.81-0.76 (m, 2H).

Z4: tert-butyl 4-[4-(2-chloro-5-fluoropyrimidin-4-yl)-2-cyanophenoxy]-3-fluoropiperidine-1-carboxylate: To a mixture of Z3 (19.0 g, 42.6 mmol, 1.00 eq), 3A (7.11 g, 42.6 mmol, 1 eq) and K₂CO₃ (17.7 g, 128.0 mmol, 3.00 eq) in 1-4dioxane (150 mL) and H₂O (7.5 mL) was added Pd(dppf)Cl₂.CH₂Cl₂ (1.74 g, 2.13 mmol, 0.05 eq) under N₂. The mixture was then stirred at 90° C. for 2 h. The mixture was washed with water (500 mL) and then extracted with EtOAc (300 mL×3), the combined organic phase was dried with anhydrous Na₂SO₄, filtered and concentrated to give a crude product. The crude product was purified by silica gel chromatography with petroleum ether: EtOAc from 50:1 to 10:1 to give the title compound (13.0 g, 28.7 mmol, 67.4% yield, 99.5% purity) as an yellow oil. ¹HNMR: (CDCl₃, 400 MHz). δ 8.48 (d, J=3.6 Hz, 1H), 8.38 (d, J=2.4 Hz, 1H), 8.33 (d, J=2.4, 9.2 Hz, 1H), 7.15 (d, J=8.8 Hz, 1H), 4.92-4.88 (m, 2H), 3.94-3.39 (m, 4H), 2.10-2.06 (m, 1H), 1.85-1.82 (m, 1H), 1.57 (s, 9H); LCMS: RT=1.004 min, m/z (M+Na⁺)=473.2.

Z5: tert-butyl 4-{2-cyano-4-[5-fluoro-2-({4-[4-(oxetan-3-yl)piperazin-1-yl]phenyl}amino)pyrimidin-4-yl]phenoxy}-3-fluoropiperidine-1-carboxylate: To a solution of compound Z4 (531.0 mg, 1.18 mmol, 1.10 eq) in 1,4-dioxane (20 mL) was added compound B1 (250 mg, 1.07 mmol, 1.00 eq), BINAP (102.0 mg, 214.0 umol, 0.20 eq), Pd(dba)₂ (123.0 mg, 214.0 umol, 0.200 eq) and Cs₂CO₃ (697.0 mg, 2.14 mmol, 2.00 eq). The reaction mixture was diluted with H₂O (20 mL) and extracted with EtOAc 90 mL (30 mL×3). The combined organic layers were washed with brine (50 mL), dried over Na₂SO₄. filtered and concentrated under reduced pressure to give the title compound as a brown oil (500 mg) which was used in the next step directly. LCMS: RT=0.890 min, m/z (M+H⁺)=648.6.

Z6:5-[5-fluoro-2-({4-[4-(oxetan-3-yl)piperazin-1-yl]phenyl}amino)pyrimidin-4-yl]-2-[(3-fluoropiperidin-4-yl)oxy]benzonitrile: To a solution of compound Z5 (500.0 mg, 772.0 umol, 1.00 eq) was dissolved in EtOAc (150 mL), Then added HCl (12.0 M, 23.3 mL, 362.4 eq) and H₂O (110 mL). The mixture was stirred at 30° C. for 6 h. The aqueous layer was neutralized with sat.Na₂CO₃ (pH=9), Then exacted with EtOAc 300 mL (100 mL×3), The combined organic layers were washed with brine 100 mL, dried over Na₂SO₄, filtered and concentrated under reduced pressure to give The title compound (600.0 mg) which was used in the next step directly. LCMS: RT=0.748 min, m/z (M+H⁺)=548.4.

2-{[(3R,4S)-3-fluoro-1-(2-hydroxypropanoyl)piperidin-4-yl]oxy}-5-[5-fluoro-2-({4-[4-(oxetan-3-yl)piperazin-1-yl]phenyl}amino)pyrimidin-4-yl]benzonitrile: To a solution of 5-[5-fluoro-2-({4-[4-(oxetan-3-yl)piperazin-1-yl]phenyl}amino)pyrimidin-4-yl]-2-[(3-fluoropiperidin-4-yl)oxy]benzonitrile (400.0 mg, 730.0 umol, 1.00 eq) in DMF (10 mL) was added compound 2-hydroxypropanoic acid, 6A (131.0 mg, 1.46 mmol, 109.0 uL, 2.00 eq), HATU (556.0 mg, 1.46 mmol, 2.00 eq) and DIPEA (189.0 mg, 1.46 mmol, 254.0 uL, 2.00 eq). The mixture was stirred at 30° C. for 2 h. The reaction mixture was diluted with EtOAc (30 mL) and washed with H₂O 150 mL (50 mL×3). The organic layers were washed with brine (50 mL), dried over Na₂SO₄, filtered and concentrated under reduced pressure to give black brown oil. The crude was purified by prep-HPLC (column: Boston Green ODS 150×30×5u; mobile phase: [water (0.225% FA)−ACN]; B %: 15%-45%, 10 min) to obtain the title compound as an yellow solid (88.5 mg, 127.1 umol, 17.4% yield, 95.5% purity) LCMS: RT=0.812 min, m/z (M+H⁺)=620.4; HPLC: RT=6.98, 95.6% purity; ¹HNMR: (400MHZ, CDCl₃) δ 8.42 (s, 1H), 8.30-8.38 (m, 2H), 7.45-7.52 (m, 2H), 7.21 (s, 1H), 6.97 (d, 7=9.03 Hz, 2H), 5.01 (d, 7=11.54 Hz, 1H), 4.86 (s, 1H), 4.69-4.75 (m, 4H), 4.47-4.57 (m, 1H), 4.11 (d, 7=14.80 Hz, 1H), 3.93 (d, 7=8.78 Hz, 1H), 3.67-3.77 (m, 1H), 3.61 (td, 7=6.49, 12.86 Hz, 2H), 3.18-3.28 (m, 4H), 2.49-2.62 (m, 4H), 2.36 (s, 2H), 2.18 (d, 7=4.78 Hz, 1H), 1.95 (s, 1H), 1.32-1.44 (m, 3H).

### Example 296: 2-({3-fluoro-1-[(2S)-2-hydroxypropanoyl]piperidin-4-yl}oxy)-5-[5-fluoro-2-({4-[4-(oxetan-3-yl)piperazin-1-yl]phenyl}amino)pyrimidin-4-yl]benzonitrile

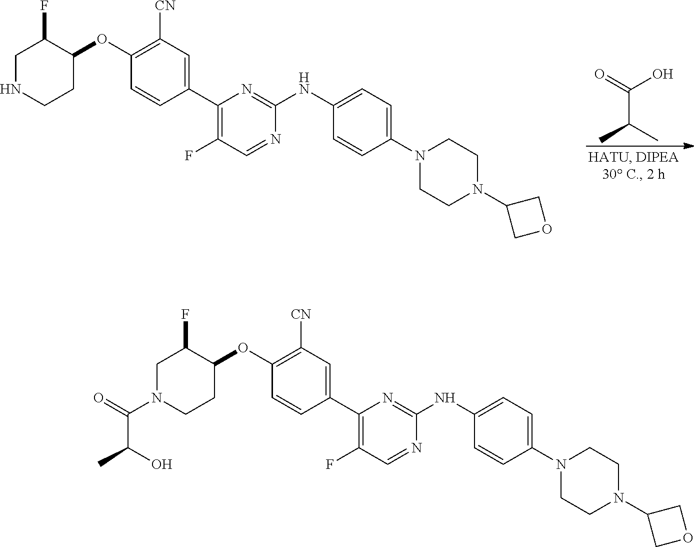

The title compound was prepared from 5-[5-fluoro-2-({4-[4-(oxetan-3-yl)piperazin-1-yl]phenyl}amino)pyrimidin-4-yl]-2-[(3-fluoropiperidin-4-yl)oxy]benzonitrile (250.0 mg, 457.0 umol, 1.00 eq) and (S)-2-hydroxypropanoic acid (82.3 mg, 913.0 umol, 67.9 uL, 2.00 eq) using Method A. The product was purified by prep-HPLC to obtain the title compound (60.9 mg, 9.89%) was obtained as an yellow solid. LCMS: RT=0.925 min, m/z (M+H⁺)=620.5; HPLC: RT=7.02, 92.8% purity; ¹HNMR: (400MHZ, CDCl₃) δ 8.42 (s, 1H), 8.30-8.38 (m, 2H), 7.45-7.52 (m, 2H), 7.21 (s, 2H), 6.97 (d, J=9.00 Hz, 2H), 5.01 (d, J=11.54 Hz, 1H), 4.86 (s, 1H), 4.69-4.75 (m, 4H), 4.47-4.57 (m, 1H), 4.11 (d, J=14.80 Hz, 1H), 3.93 (d, J=8.78 Hz, 1H), 3.67-3.77 (m, 1H), 3.61 (td, J=6.49, 12.86 Hz, 1H), 3.18-3.28 (m, 4H), 2.49-2.62 (m, 4H), 2.36 (s, 2H), 2.18 (d, 7=4.78 Hz, 1H), 1.95 (s, 1H), 1.32-1.44 (m, 3H).

### Example 297: 2-{[3-fluoro-1-(2-hydroxypropanoyl)piperidin-4-yl]oxy}-5-[5-fluoro-2-({6-methoxy-5-[4-(oxetan-3-yl)piperazin-1-yl]pyridin-2-yl}amino)pyrimidin-4-yl]benzonitrile

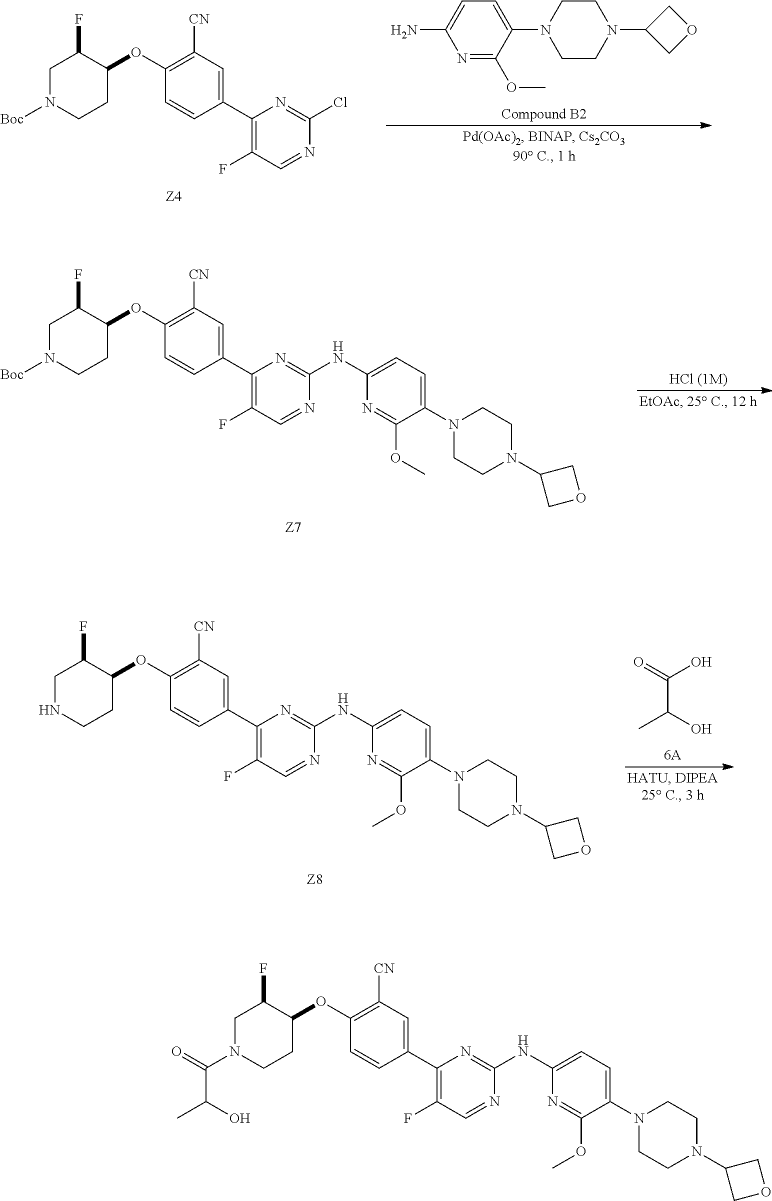

Z7: tert-butyl 4-{2-cyano-4-[5-fluoro-2-({6-methoxy-5-[4-(oxetan-3-yl)piperazin-1-yl]pyridin-2-yl}amino)pyrimidin-4-yl]phenoxy}-3-fluoropiperidine-1-carboxylate: A mixture of compound Z4 (1.05 g, 2.32 mmol, 1.1 eq), 6-methoxy-5-[4-(oxetan-3-yl)piperazin-1-yl]pyridin-2-amine (0.60 g, 2.11 mmol, 1 eq), Cs₂CO₃ (1.38 g, 4.22 mmol, 2 eq), Pd(OAc)₂ (94.8 mg, 422.0 umol, 0.2 eq), BINAP (263.0 mg, 422.0 umol, 0.2 eq) in dioxane (10 mL) was degassed and purged with N₂ for 3 times, and then the mixture was stirred at 90° C. for 1 h under N₂ atmosphere. The reaction was filtered and the filtrate was diluted with H₂O (10 mL) and extracted with EtOAc (20 mL×3). The combined organic layers were washed with saturated brines (20 mL×3), dried over anhydrous Na₂SO₄, filtered and concentrated under reduced pressure to give Z7 as an yellow solid (2 g, crude). LCMS: RT=1.072 min, MS (M+H⁺): 679.4.

Z8:5-[5-fluoro-2-({6-methoxy-5-[4-(oxetan-3-yl)piperazin-1-yl]pyridin-2-yl}amino)pyrimidin-4-yl]-2-[(3-fluoropiperidin-4-yl)oxy]benzonitrile: To a solution of tert-butyl 4-{2-cyano-4-[5-fluoro-2-({6-methoxy-5-[4-(oxetan-3-yl)piperazin-1-yl]pyridin-2-yl}amino)pyrimidin-4-yl]phenoxy}-3-fluoropiperidine-1-carboxylate, Z7 (1.43 g, 2.11 mmol, 1 eq) in EtOAc (20 mL) was added HCl (1 M, 20 mL, 9.49 eq). The mixture was stirred at 25° C. for 12 h. The reaction mixture was adjusted to pH=7˜8 with saturated NaHCO₃ and was extracted with EtOAc (50 mL×3) to remove less polar impurities. Then aqueous phase was extracted with DCM (80 mL×3), the combined organic layers were washed with brine (80 mL×3), dried over anhydrous Na₂SO₄, filtered and concentrated under reduced pressure to give the title compound (0.60 g) as an yellow solid which was for the next step directly. LCMS: RT=0.948 min, MS (M+H⁺): 579.5.

2-{[3-fluoro-1-(2-hydroxypropanoyl)piperidin-4-yl]oxy}-5-[5-fluoro-2-({6-methoxy-5-[4-(oxetan-3-yl)piperazin-1-yl]pyridin-2-yl}amino)pyrimidin-4-yl]benzonitrile: To a solution of compound Z8 (0.26 g, 449.0 umol, 1 eq) in DMF (3 mL) was added compound 6A (56.7 mg, 629.0 umol, 46.8 uL, 1.4 eq), HATU (188.0 mg, 494.0 umol, 1.1 eq), DIPEA (87.1 mg, 674.0 umol, 117.0 uL, 1.5 eq) was stirred at 25° C. for 3 h. The reaction was diluted with H₂O (15 mL) and extracted with DCM (30 mL×3). The combined organic layers were washed with water (30 mL×3), dried over anhydrous Na₂SO₄, filtered and concentrated under reduced pressure to give a residue. The residue was triturated with MTBE (5 mL) and to give the title compound (46.1 mg, 66.3 umol, 14.8% yield, 93.5% purity) as a yellow solid. LCMS: RT=0.907 min, MS (M+H⁺): 651.4; HPLC: RT=2.225 min, 93.5% purity; ¹H NMR: (400 MHz, DMSO-de) δ: 9.56 (s, 1H), 8.76 (d, J=3.2 Hz, 1H), 8.41-8.34 (m, 2H), 7.67-7.61 (m, 2H), 7.25 (d, J=8.4 Hz, 1H), 5.16-4.99 (m, 3H), 4.57-4.44 (m, 5H), 4.36-3.93 (m, 2H), 3.87 (s, 3H), 3.71-3.56 (m, 1H), 3.49-3.43 (m, 1H), 2.97 (s, 4H), 2.40 (s, 4H), 2.08-1.87 (m, 3H), 1.22 (d, J=6.4 Hz, 3H).

### Example 298: 2-({3-fluoro-1-[(2S)-2-hydroxypropanoyl]piperidin-4-yl}oxy)-5-[5-fluoro-2-({6-methoxy-5-[4-(oxetan-3-yl)piperazin-1-yl]pyridin-2-yl}amino)pyrimidin-4-yl]benzonitrile

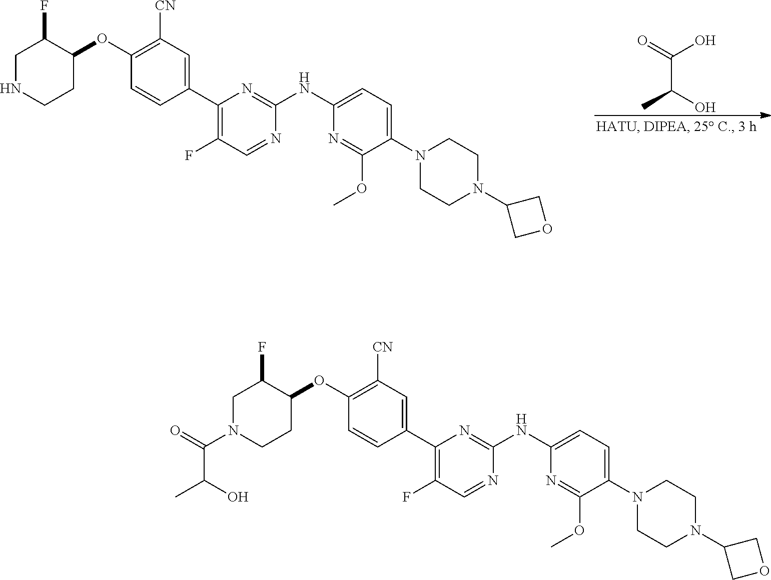

The title compound was prepared from 5-[5-fluoro-2-({6-methoxy-5-[4-(oxetan-3-yl)piperazin-1-yl]pyridin-2-yl}amino)pyrimidin-4-yl]-2-[(3-fluoropiperidin-4-yl)oxy]benzonitrile (0.26 g, 449.0 umol, 1 eq) and (S)-2-hydroxypropanoic acid (56.7 mg, 629.0 umol, 46.8 uL, 1.4 eq) using Method A. The product was purified by prep-HPLC to obtain the title compound (59.05 mg, 85.5 umol, 19.0% yield, 94.2% purity) as a yellow solid. LCMS: RT=0.917 min, MS (M+H⁺): 651.4; HPLC: RT=2.239 min, 94.2% purity; ¹H NMR: (400 MHz, DMSO-d₆) δ: 9.56 (s, 1H), 8.67 (d, J=3.2 Hz, 1H), 8.40-8.34 (m, 2H), 7.67-7.61 (m, 2H), 7.25 (d, J=8.4 Hz, 1H), 5.16-4.99 (m, 3H), 4.57-4.54 (m, 5H), 4.47-4.45 (m, 2H), 3.89 (s, 3H), 3.48-3.45 (m, 1H), 3.43-3.33 (m, 1H), 2.97 (s, 4H), 2.40 (s, 4H), 2.08-2.00 (m, 3H), 1.22 (d, J=6.4 Hz, 3H).

### Example 299:2-{[3,3-difluoro-1-(2-hydroxypropanoyl)piperidin-4-yl]oxy}-5-[5-fluoro-2-({4-[4-(oxetan-3-yl)piperazin-1-yl]phenyl}amino)pyrimidin-4-yl]benzonitrile

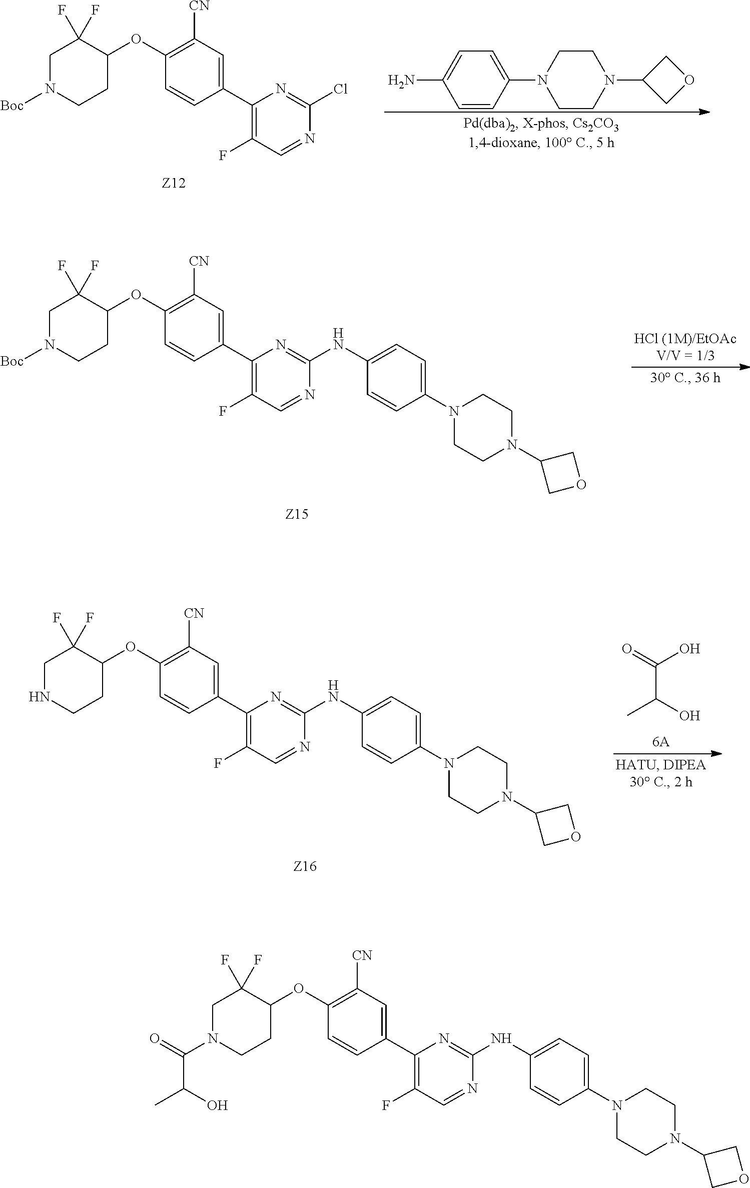

The title compound was synthesized using the procedures as in Example 293 starting from tert-butyl 4-[4-(2-chloro-5-fluoropyrimidin-4-yl)-2-cyanophenoxy]-3,3-difluoropiperidine-1-carboxylate and 4-[4-(oxetan-3-yl)piperazin-1-yl]aniline. The final compound crude was purified by prep-HPLC (column: Luna C18 150×25×5u; mobile phase: [water (0.225% FA)−ACN]; B %: 14%-44%, 10 min) to obtain an yellow solid. LCMS: RT=0.884 min, m/z (M+H⁺)=638.3; HPLC: EW8892-14-P1C, RT=7.33 min, 98.9% purity; ¹HNMR: (400MHZ, CDCl₃) δ 8.43 (s, 1H), 8.33-8.36 (s, 1H), 8.07 (s, 1H), 7.46-7.53 (m, 2H), 7.19-7.25 (s, 2H), 6.94-6.98 (m, 2H), 5.07-5.29 (m, 1H), 4.88 (s, 1H), 4.68-4.75 (m, 4H), 4.54 (dd, 7=6.66, 13.18 Hz, 2H), 3.86-3.98 (m, 1H), 3.67-3.77 (m, 1H), 3.59-3.64 (m, 1H), 3.20-3.27 (m, 4H), 2.54-2.60 (m, 4H), 1.47-1.63 (m, 3H), 1.37-1.46 (m, 3H).

### Example 300:2-({3,3-difluoro-1-[(2S)-2-hydroxypropanoyl]piperidin-4-yl}oxy)-5-[5-fluoro-2-({4-[4-(oxetan-3-yl)piperazin-1-yl]phenyl}amino)pyrimidin-4-yl]benzonitrile

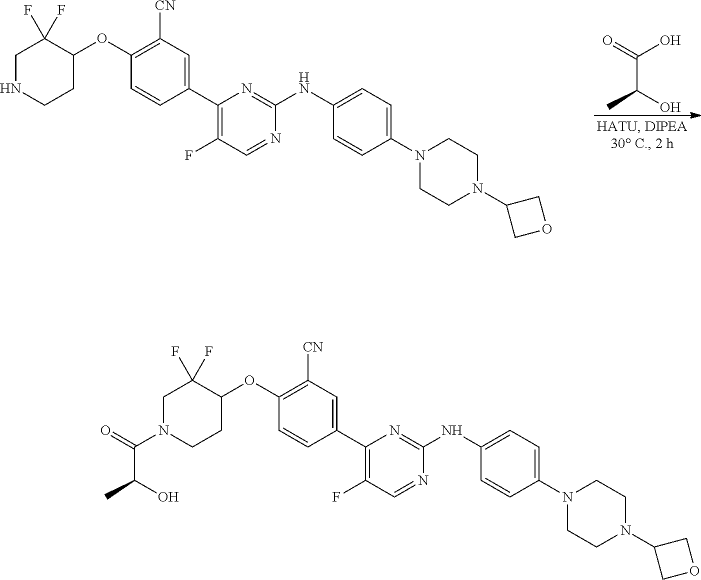

The title compound was prepared from 2-[(3,3-difluoropiperidin-4-yl)oxy]-5-[5-fluoro-2-({4-[4-(oxetan-3-yl)piperazin-1-yl]phenyl}amino)pyrimidin-4-yl]benzonitrile (200.0 mg, 354.0 umol, 1.00 eq) and (S)-2-hydroxypropanoic acid (63.7 mg, 707.0 umol, 52.7 uL, 2.00 eq) using Method A. The product was purified by prep-HPLC to obtain the title compound (32.8 mg, 13.1%) as a yellow solid. LCMS: RT=0.894 min, m/z (M+H⁺)=638.4; HPLC: RT=7.31 min, 96.7% purity; ¹HNMR: (400MHZ, CDCl₃) δ 8.43 (s, 1H), 8.33-8.36 (s, 1H), 8.07 (s, 1H), 7.46-7.53 (m, 2H), 7.19-7.25 (s, 2H), 6.94-6.98 (m, 2H), 5.20-5.24 (m, 1H), 4.88 (s, 1H), 4.68-4.75 (m, 4H), 4.54 (dd, 7=6.66, 13.18 Hz, 2H), 3.90-3.96 (m, 1H), 3.60-3.63 (m, 2H), 3.23-3.25 (m, 4H), 2.58-2.64 (m, 4H), 1.55-1.61 (m, 3H), 1.38-1.55 (m, 3H).

### Example 301: 2-({3,3-difluoro-1-[(2S)-2-hydroxypropanoyl]piperidin-4-yl}oxy)-5-[5-fluoro-2-({4-[1-(oxetan-3-yl)piperidin-4-yl]phenyl}amino)pyrimidin-4-yl]benzonitrile

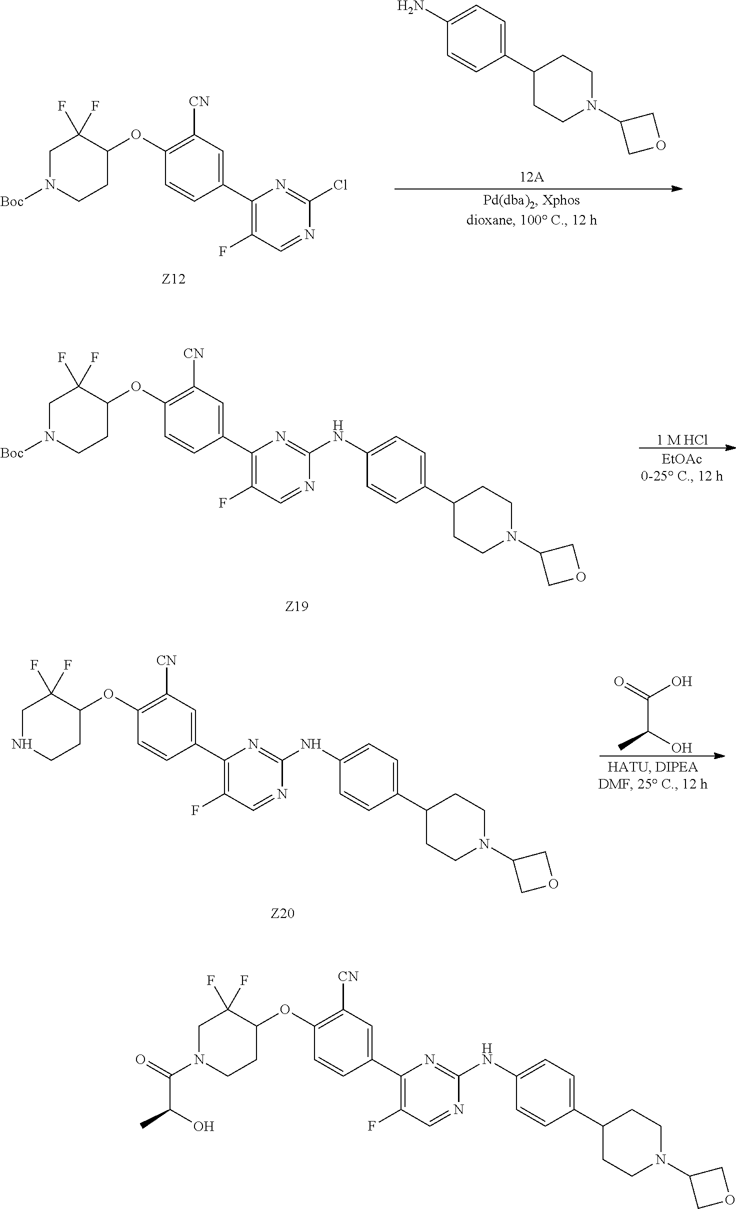

The title compound was synthesized using the procedures as in Example 293 starting from tert-butyl 4-[4-(2-chloro-5-fluoropyrimidin-4-yl)-2-cyanophenoxy]-3,3-difluoropiperidine-1-carboxylate and 4-[1-(oxetan-3-yl)piperidin-4-yl]aniline. The crude product was purified by pre-HPLC (column: Phenomenex Gemini 150×25 mm×10 um; mobile phase: [water (10 mM NH₄HCO₃)-ACN]; B %: 38%-68%, 10 min) and lyophilized to give 2-({3,3-difluoro-1-[(2S)-2-hydroxypropanoyl]piperidin-4-yl}oxy)-5-[5-fluoro-2-({4-[1-(oxetan-3-yl)piperidin-4-yl]phenyl}amino)pyrimidin-4-yl]benzonitrile (18.0 mg, 19.9%, 100% purity) as an yellow solid. LCMS: RT=0.951 min, MS: [M+1]⁺, 637.3; HPLC: RT=3.314 min, 100% purity; ¹HNMR: (CDCl₃, 400 MHz) δ 8.47-8.36 (m, 3H), 7.54 (d, J=8.4 Hz, 2H), 7.24 (s, 3H), 7.14 (s, 1H), 4.89 (s, 1H), 4.73-4.65 (m, 4H), 4.54 (s, 1H), 4.05-3.84 (m, 1H), 3.69 (s, 2H), 3.52 (t, J=6.4 Hz, 1H), 2.90 (d, J=11.6 Hz, 2H), 2.62-2.42 (m, 1H), 2.20 (m, 2H), 2.01-1.76 (m, 6H), 1.54 (s, 1H), 1.36-1.35 (m, 3H).

### Example 303: 5-([33-difluoro-1-[(2S)-2-hydroxypropanoyl]piperidin-4-yl]oxy)-2-[2-([6-methoxy-5-[4-(oxetan-3-yl)piperazin-1-yl]pyridin-2-yl]amino)pyrimidin-4-yl]pyridine-4-carbonitrile

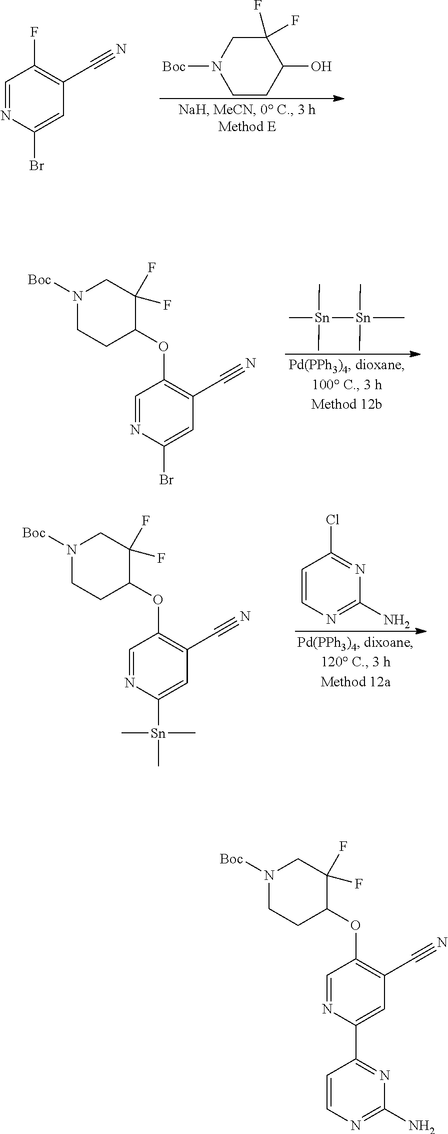

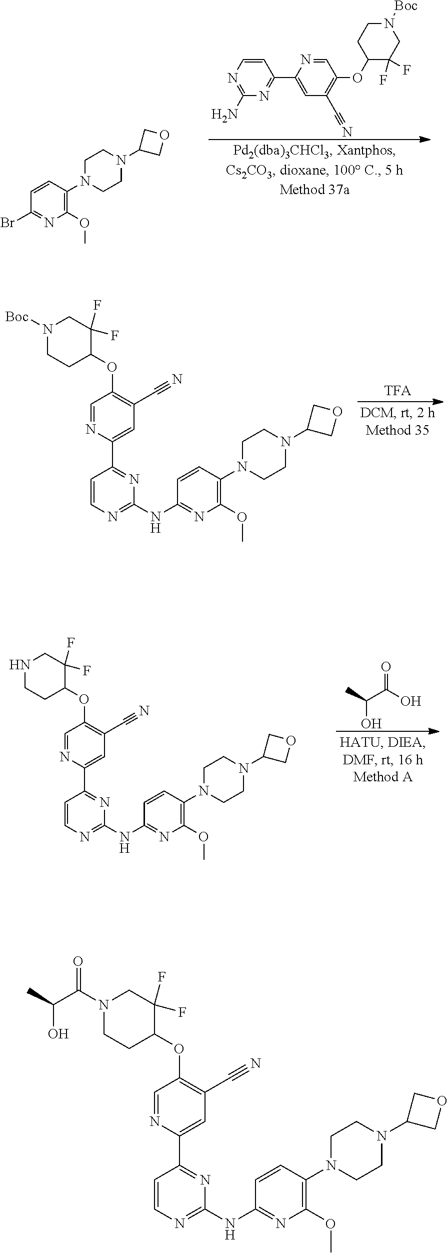

The title compound was prepared from 2-bromo-5-fluoroisonicotinonitrile, tert-butyl 3,3-difluoro-4-hydroxypiperidine-1-carboxylate, 1,1,1,2,2,2-hexamethyldistannane, 4-chloropyrimidin-2-amine, 1-(6-bromo-2-methoxypyridin-3-yl)-4-(oxetan-3-yl)piperazine, and (S)-2-hydroxypropanoic acid using Method E, 12a, 12b, 37a, 35 and A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 30% to 50% gradient in 8 min; detector, UV 254 nm. 5-([3,3-difluoro-1-[(2S)-2-hydroxypropanoyl]piperidin-4-yl]oxy)-2-[2-([6-methoxy-5-[4-(oxetan-3-yl)piperazin-1-yl]pyridin-2-yl]amino)pyrimidin-4-yl]pyridine-4-carbonitrile was obtained as an yellow solid (17 mg, 3.6% for 6 steps). HPLC: 94.7% purity, RT=6.45 min. MS: m/z=652.1[M+H]⁺. ¹H NMR (300 MHz, Chloroform-d, ppm) δ 8.71-8.58 (m, 3H), 7.89-7.80 (m, 1H), 7.75-7.65 (m, 2H), 7.33-7.24 (m, 1H), 5.04-4.93 (m, 1H), 4.87-4.32 (m, 6H), 3.98 (s, 3H), 3.92-3.86 (m, 1H), 3.76-3.28 (m, 4H), 3.16-3.10 (m, 4H), 2.60-2.53 (m, 4H), 2.29-2.23 (m, 2H), 1.52-1.34 (m, 3H).

### Example 304: 3-([3,3-difluoro-1-[(2S)-2-hydroxypropanoyl]piperidin-4-yl]oxy)-6-[2-([6-methoxy-5-[4-(oxetan-3-yl)piperazin-1-yl]pyridin-2-yl]amino)pyrimidin-4-yl]pyridine-2-carbonitrile

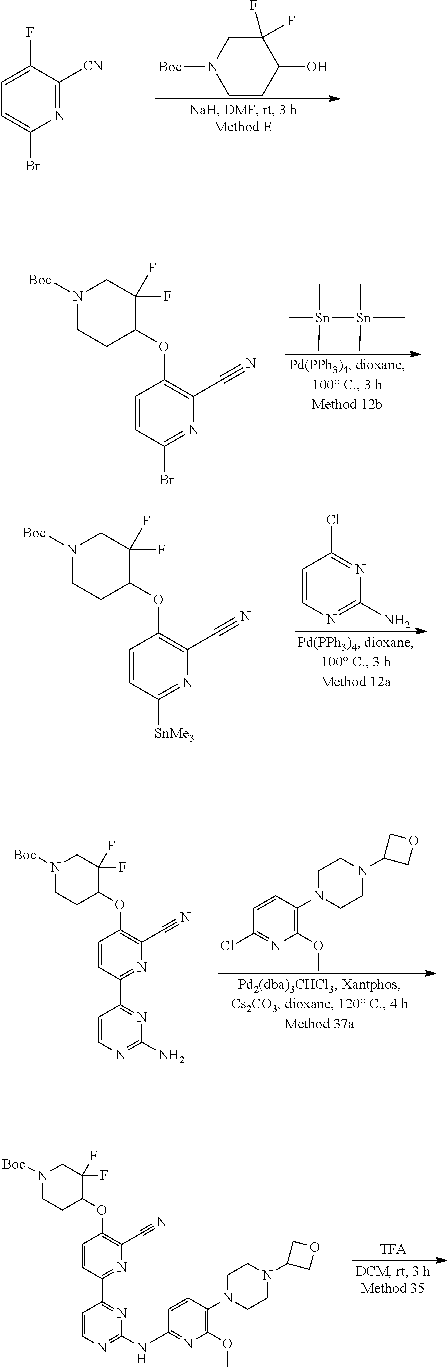

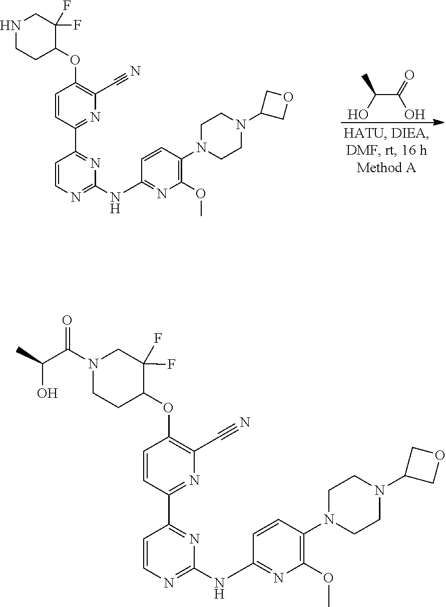

3-[(3,3-difluoropiperidin-4-yl)oxy]-6-[2-([6-methoxy-5-[4-(oxetan-3-yl)piperazin-1-yl]pyridin-2-yl]amino)pyrimidin-4-yl]pyridine-2-carbonitrile: The title compound was prepared from tert-butyl 3,3-difluoro-4-hydroxypiperidine-1-carboxylate, 6-bromo-3-fluoropyridine-2-carbonitrile, 4-chloropyrimidin-2-amine and 1-(6-chloro-2-methoxypyridin-3-yl)-4-(oxetan-3-yl)piperazine using Method E, 12b, 12a, 37a and 35. The final product was purified by prep-HPLC under the following condition: column, Atlantis HILIC OBD C18 Column, 150×19 mm, 5 um; mobile phase, acetonitrile in water (with 10 mmol/L NH₄HCO₃ and 0.1% NH₃.H₂O), 30% to 50% gradient in 8 min; detector, UV 254 nm. 3-[(3,3-difluoropiperidin-4-yl)oxy]-6-[2-([6-methoxy-5-[4-(oxetan-3-yl)piperazin-1-yl]pyridin-2-yl]amino)pyrimidin-4-yl]pyridine-2-carbonitrile was obtained as an yellow solid (8 mg, 2.7% for 5 steps). HPLC: 99.7% purity, RT=3.72 min. MS: m/z=580.2 [M+H]⁺. ¹H NMR (300 MHz, DMSO-d₆, ppm) δ 9.52 (s, 1H), 8.71-8.61 (m, 2H), 8.28-8.18 (m, 1H), 7.81-7.71 (m, 1H), 7.67-7.59 (m, 1H), 7.34-7.24 (m, 1H), 5.30-5.23 (m, 1H), 4.62-4.41 (m, 4H), 3.90 (s, 3H), 3.54-3.41 (m, 1H), 3.22-3.08 (m, 1H), 3.02-2.85 (m, 6H), 2.73-2.66 (m, 2H), 2.45-2.38 (m, 4H), 2.16-1.77 (m, 2H).

3-([3,3-difluoro-1-[(2S)-2-hydroxypropanoyl]piperidin-4-yl]oxy)-6-[2-([6-methoxy-5-[4-(oxetan-3-yl)piperazin-1-yl]pyridin-2-yl]amino)pyrimidin-4-yl]pyridine-2-carbonitrile: The title compound was prepared from 3-[(3,3-difluoropiperidin-4-yl)oxy]-6-[2-([6-methoxy-5-[4-(oxetan-3-yl)piperazin-1-yl]pyridin-2-yl]amino)pyrimidin-4-yl]pyridine-2-carbonitrile and (2S)-2-hydroxypropanoic acid using Method A. The final product was purified by prep-HPLC under the following condition: column, Atlantis HILIC OBD C18 Column, 150×19 mm, 5 um; mobile phase, acetonitrile in water (with 10 mmol/L NH₄HCO₃ and 0.1% NH₃.H₂O), 22% to 51% gradient in 8 min; detector, UV 254 nm. 3-([3,3-difluoro-1-[(2S)-2-hydroxypropanoyl]piperidin-4-yl]oxy)-6-[2-([6-methoxy-5-[4-(oxetan-3-yl)piperazin-1-yl]pyridin-2-yl]amino)pyrimidin-4-yl]pyridine-2-carbonitrile was obtained as an yellow solid (30 mg, 27%). HPLC: 95.0% purity, RT=3.71 min. MS: m/z=652.2 [M+H]⁺. ¹H NMR (300 MHz, DMSO-d₆, ppm) δ 9.53 (s, 1H), 8.73-8.63 (m, 2H), 8.31-8.21 (m, 1H), 7.81-7.71 (m, 1H), 7.68-7.60 (m, 1H), 7.34-7.24 (m, 1H), 5.46-5.40 (m, 1H), 5.31-5.21 (m, 1H), 4.64-4.40 (m, 5H), 4.35-3.95 (m, 1H), 3.90 (s, 3H), 3.88-3.37 (m, 4H), 3.10-2.87 (m, 4H), 2.45-2.38 (m, 4H), 2.35-1.89 (m, 2H), 1.23 (d, J=6.4 Hz, 3H).

### Example 305: 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]pyridine-3-carbonitrile

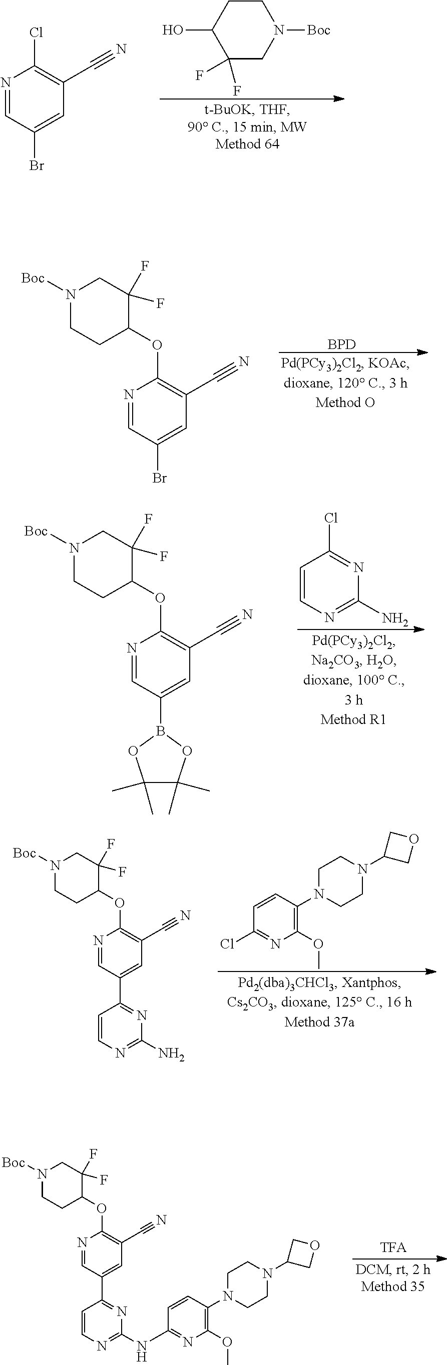

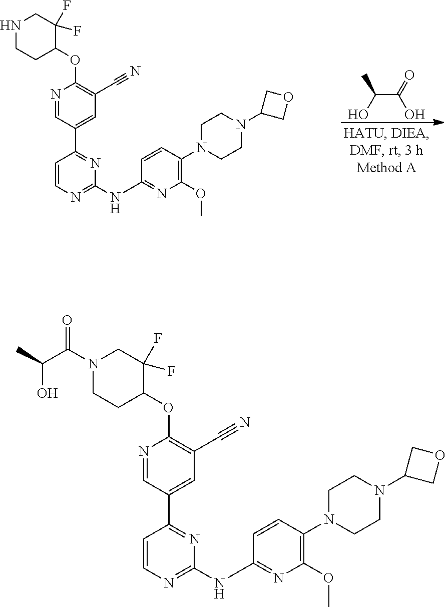

### Method 64

2-(azetidin-3-yloxy)-5-[2-([4-[4-(oxetan-3-yl)piperazin-1-yl]phenyl]amino)pyrimidin-4-yl]benzonitrile: To a solution of 5-bromo-2-chloropyridine-3-carbonitrile (950 mg, 4.37 mmol) in THF (15 mL) was added tert-butyl 3,3-difluoro-4-hydroxypiperidine-1-carboxylate (1250 mg, 5.27 mmol), and t-BuOK (1230 mg, 10.97 mmol) at room temperature. The reaction mixture was irradiated with microwave for 15 min at 90° C. When the reaction was done, the solids were filtered out and the filtrate was concentrated under reduced pressure. The residue was purified by flash chromatography eluting with EtOAc in hexane (0% to 10% gradient) to yield tert-butyl 4-[(5-bromo-3-cyanopyridin-2-yl)oxy]-3,3-difluoropiperidine-1-carboxylate as a light yellow oil (725 mg, 39%). MS: m/z=440.0 [M+H]⁺.

2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]pyridine-3-carbonitrile: The title compound was prepared from tert-butyl 4-(5-bromo-3-cyanopyridin-2-yloxy)-3,3-difluoropiperidine-1-carboxylate, BPD, 4-chloropyrimidin-2-amine, 1-(6-chloro-2-methoxypyridin-3-yl)-4-(oxetan-3-yl)piperazine, and (S)-2-hydroxypropanoic acid using Method O, R1, 37a, 35 and A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 30% to 50% gradient in 8 min; detector, UV 254 nm. 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]pyridine-3-carbonitrile was obtained as a light yellow solid (30 mg, 7.4% for 5 steps). HPLC: 94.0% purity, RT=9.42 min. MS: m/z=652.2 [M+H]⁺. ¹H NMR (400 MHz, DMSO-d₆, ppm) δ 9.44 (s, 1H), 9.28-9.23 (m, 1H), 9.08-9.03 (m, 1H), 8.67-8.61 (m, 1H), 7.76-7.69 (m, 1H), 7.59-7.53 (m, 1H), 7.33-7.27 (m, 1H), 5.94-5.90 (m, 1H), 5.22 (d, J=6.8 Hz, 1H), 4.60-4.43 (m, 5H), 4.33-4.19 (m, 1H), 4.10-3.93 (m, 2H), 3.90 (s, 3H), 3.86-3.57 (m, 1H), 3.53-3.43 (m, 1H), 3.01-2.97 (m, 4H), 2.43-2.39 (m, 4H), 2.29-1.75 (m, 2H), 1.27-1.20 (m, 3H).

### Example 306: 3-([33-difluoro-1-[(2S)-2-hydroxypropanoyl]piperidin-4-yl]oxy)-6-[2-([4-[4-(oxetan-3-yl)piperazin-1-yl]phenyl]amino)pyrimidin-4-yl]pyridine-2-carbonitrile

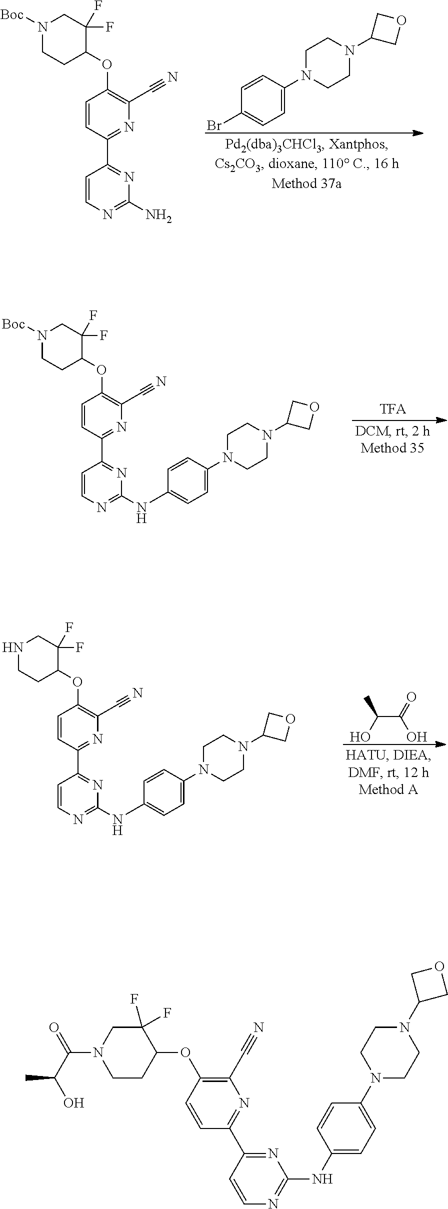

3-[(3,3-difluoropiperidin-4-yl)oxy]-6-[2-([4-[4-(oxetan-3-yl)piperazin-1-yl]phenyl]amino)pyrimidin-4-yl]pyridine-2-carbonitrile: The title compound was prepared from tert-butyl 4-(6-(2-aminopyrimidin-4-yl)-2-cyanopyridin-3-yloxy)-3,3-difluoropiperidine-1-carboxylate and 1-(4-bromophenyl)-4-(oxetan-3-yl)piperazine using Method 37a and 35. The final product was purified by prep-HPLC under the following condition: column, XBridge Prep OBD C18 Column, 150×30 mm, 5 um; mobile phase, acetonitrile in water (with 10 mmol/L NR₄HCCO₃ and 0.1% NH₃.H₂O), 18% to 42% gradient in 8 min; detector, UV 254 nm. 3-[(3,3-difluoropiperidin-4-yl)oxy]-6-[2-([4-[4-(oxetan-3-yl)piperazin-1-yl]phenyl]amino)pyrimidin-4-yl]pyridine-2-carbonitrile was obtained as a light yellow solid (16 mg, 16% for 2 steps). HPLC: 98.7% purity, RT=3.68 min. MS: m/z=549.1[M+H]⁺. ¹H NMR (300 MHz, DMSO-d₆, ppm) δ 9.51 (s, 1H), 8.61-8.51 (m, 2H), 8.24-8.15 (m, 1H), 7.66-7.55 (m, 2H), 7.53-7.45 (m, 1H), 6.96-6.87 (m, 2H), 5.26-5.20 (m, 1H), 4.61-4.50 (m, 2H), 4.51-4.41 (m, 2H), 3.51-3.36 (m, 1H), 3.25-3.05 (m, 5H), 3.01-2.81 (m, 2H), 2.71-2.60 (m, 2H), 2.45-2.35 (m, 4H), 2.13-1.68 (m, 2H).

3-([33-difluoro-1-[(2S)-2-hydroxypropanoyl]piperidin-4-yl]oxy)-6-[2-([4-[4-(oxetan-3-yl)piperazin-1-yl]phenyl]amino)pyrimidin-4-yl]pyridine-2-carbonitrile: The title compound was prepared from 3-[(3,3-difluoropiperidin-4-yl)oxy]-6-[2-([4-[4-(oxetan-3-yl)piperazin-1-yl]phenyl]amino)pyrimidin-4-yl]pyridine-2-carbonitrile and (S)-2-hydroxypropanoic acid using Method A. The final product was purified by prep-HPLC under the following condition: column, Atlantis HILIC OBD Column, 150×19 mm, 5 um; mobile phase, acetonitrile in water (with 10 mmol/L NH₄HCO₃ and 0.1% NH₃.H₂O), 30% to 60% gradient in 8 min; detector, UV 254 nm. 3-([3,3-difluoro-1-[(2S)-2-hydroxypropanoyl]piperidin-4-yl]oxy)-6-[2-([4-[4-(oxetan-3-yl)piperazin-1-yl]phenyl]amino)pyrimidin-4-yl]pyridine-2-carbonitrile was obtained as a light yellow solid (9 mg, 11%). HPLC: 99.5% purity, RT=4.56 min. MS: m/z=621.2 [M+H]⁺. ¹H NMR (300 MHz, DMSO-d₆, ppm) δ 9.53 (s, 1H), 8.65-8.52 (m, 2H), 8.27-8.17 (m, 1H), 7.66-7.56 (m, 2H), 7.54-7.45 (m, 1H), 6.97-6.87 (m, 2H), 5.40-5.33 (m, 1H), 5.27-5.17 (m, 1H), 4.62-4.42 (m, 5H), 4.30-3.54 (m, 4H), 3.51-3.36 (m, 1H), 3.15-3.05 (m, 4H), 2.45-2.35 (m, 4H), 2.26-1.77 (m, 2H), 1.22 (d, J=6.6 Hz, 3H).

### Example 307: 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]-4-methylbenzonitrile

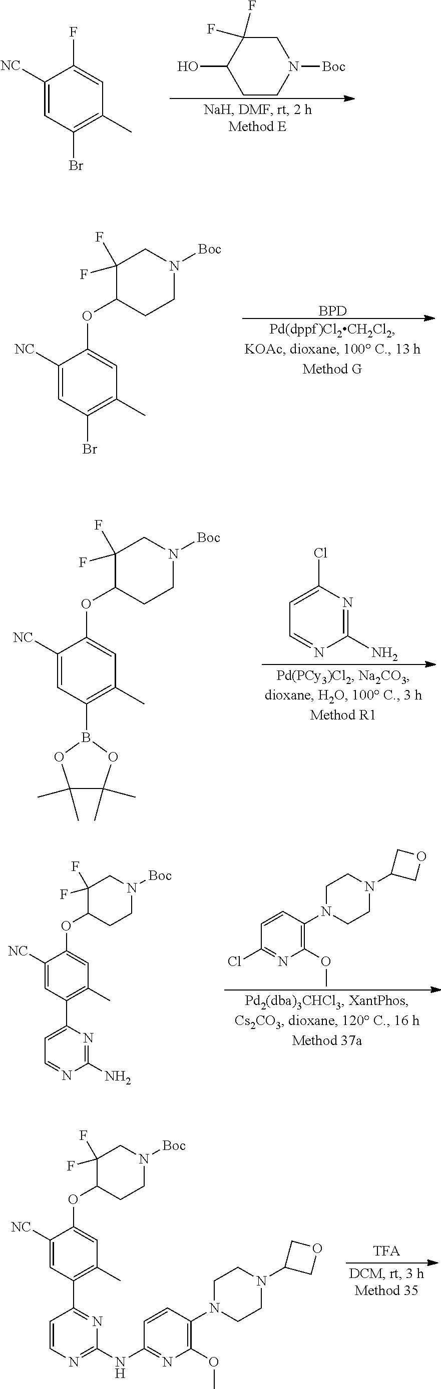

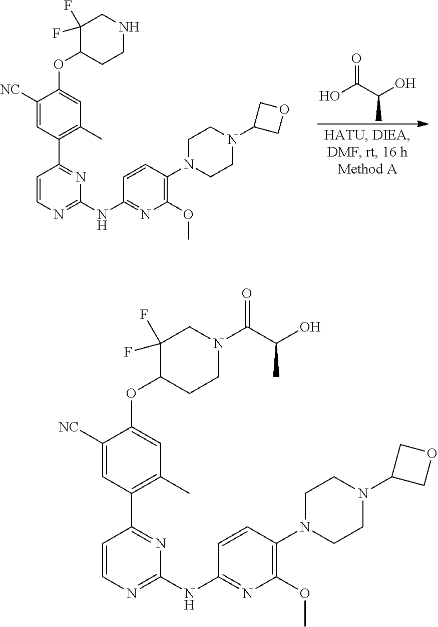

2-[(3,3-difluoropiperidin-4-yl)oxy]-5-[2-([6-methoxy-5-[4-(oxetan-3-yl)piperazin-1-yl]pyridin-2-yl]amino)pyrimidin-4-yl]-4-methylbenzonitrile: The title compound was prepared from 5-bromo-2-fluoro-4-methylbenzonitrile, tert-butyl 3,3-difluoro-4-hydroxypiperidine-1-carboxylate, BPD, 4-chloropyrimidin-2-amine, and 1-(6-chloro-2-methoxypyridin-3-yl)-4-(oxetan-3-yl)piperazine using Method E, G, R1, 37a, and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 28% to 51% gradient in 8 min; detector, UV 254 nm. 2-[(3,3-difluoropiperidin-4-yl)oxy]-5-[2-([6-methoxy-5-[4-(oxetan-3-yl)piperazin-1-yl]pyridin-2-yl]amino)pyrimidin-4-yl]-4-methylbenzonitrile was obtained as a light yellow solid (5 mg, 3.5% for 5 steps). HPLC: 99.6% purity, RT=4.42 min. MS: m/z=593.2 [M+H]⁺. ¹H NMR (300 MHz, DMSO-d₆, ppm) δ9.37 (s, 1H), 8.60-8.52 (m, 1H), 7.88 (s, 1H), 7.70-7.62 (m, 1H), 7.45 (s, 1H), 7.27-7.19 (m, 1H), 7.11-7.03 (m, 1H), 5.17-5.11 (m, 1H), 4.54 (t, J=6.5 Hz, 2H), 4.44 (t, J=6.0 Hz, 2H), 3.86 (s, 3H), 3.49-3.39 (m, 1H), 3.40-3.35 (m, 1H), 3.17-3.11 (m, 1H), 3.04-2.79 (m, 6H), 2.75-2.62 (m, 1H), 2.62-2.50 (m, 2H), 2.41-2.35 (m, 4H), 2.15-1.71 (m, 2H).

2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]-4-methylbenzonitrile: The title compound was prepared from 2-[(3,3-difluoropiperidin-4-yl)oxy]-5-[2-([6-methoxy-5-[4-(oxetan-3-yl)piperazin-1-yl]pyridin-2-yl]amino)pyrimidin-4-yl]-4-methylbenzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 26% to 56% gradient in 8 min; detector, UV 254 nm. 2-([3,3-difluoro-1-[(2S)-2-hydroxypropanoyl]piperidin-4-yl]oxy)-5-[2-([6-methoxy-5-[4-(oxetan-3-yl)piperazin-1-yl]pyridin-2-yl]amino)pyrimidin-4-yl]-4-methylbenzonitrile was obtained as off white solid (15 mg, 34%). HPLC: 97.4% purity, RT=4.13 min. MS: m/z=665.1 [M+H]⁺. ¹H NMR (300 MHz, DMSO-d₆, ppm) δ9.38 (s, 1H), 8.60-8.53 (m, 1H), 7.91 (s, 1H), 7.70-7.62 (m, 1H), 7.49 (s, 1H), 7.27-7.18 (m, 1H), 7.12-7.04 (m, 1H), 5.40-5.14 (m, 2H), 4.63-4.35 (m, 5H), 4.27-3.91 (m, 2H), 3.86 (s, 3H), 3.82-3.55 (m, 2H), 3.51-3.39 (m, 1H), 2.99-2.91 (m, 4H), 2.52 (m, 3H), 2.41-2.35 (m, 4H), 2.24-1.74 (m, 2H), 1.21 (d, J=6.5 Hz, 3H).

### Example 308: tert-butyl 4-[2-cyano-5-methyl-4-[2-([4-[4-(oxetan-3-yl)piperazin-1-yl]phenyl]amino)pyrimidin-4-yl]phenoxy]-3,3-difluoropiperidine-1-carboxylate

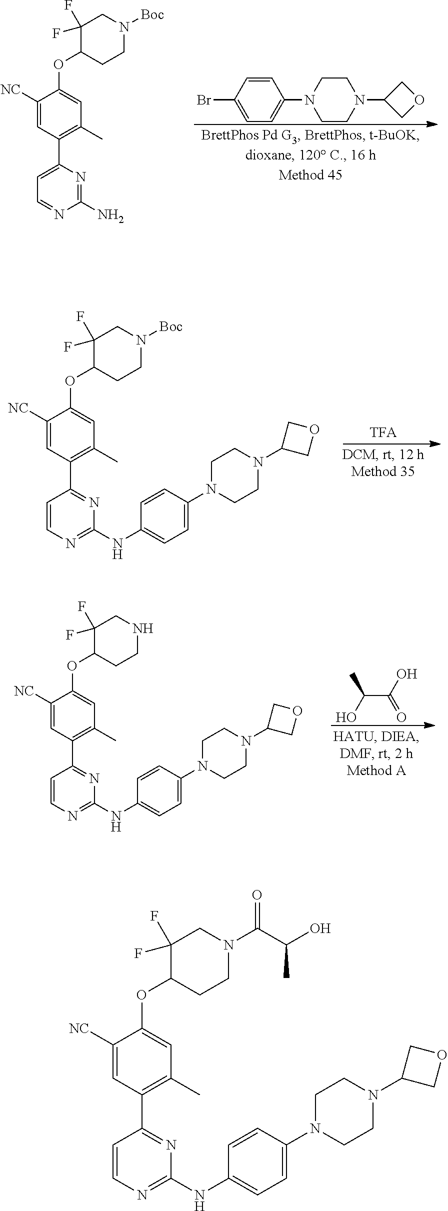

tert-butyl 4-[2-cyano-5-methyl-4-[2-([4-[4-(oxetan-3-yl)piperazin-1-yl]phenyl]amino)pyrimidin-4-yl]phenoxy]-33-difluoropiperidine-1-carboxylate: The title compound was prepared from tert-butyl 4-(4-(2-aminopyrimidin-4-yl)-2-cyano-5-methylphenoxy)-3,3-difluoropiperidine-1-carboxylate, 1-(4-bromophenyl)-4-(oxetan-3-yl)piperazine and (S)-2-hydroxypropanoic acid using Method 45, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 20% to 50% gradient in 8 min; detector, UV 254 nm. tert-butyl 4-[2-cyano-5-methyl-4-[2-([4-[4-(oxetan-3-yl)piperazin-1-yl]phenyl]amino)pyrimidin-4-yl]phenoxy]-3,3-difluoropiperidine-1-carboxylate was obtained as an yellow solid (24 mg, 7% for 3 steps). HPLC: 97.6% purity, RT=4.45 min. MS: m/z=634.2 [M+H]⁺. ¹H NMR (300 MHz, Chloroform-d, ppm) δ 8.45 (d, J=5.0 Hz, 1H), 7.72 (s, 1H), 7.53-7.44 (m, 1H), 7.13-6.87 (m, 3H), 6.74 (d, J=5.0 Hz, 1H), 4.97-4.80 (m, 1H), 4.73-4.65 (m, 4H), 4.60-4.42 (m, 2H), 4.00-3.83 (m, 1H), 3.75-3.46 (m, 4H), 3.27-3.17 (m, 4H), 2.57-2.49 (m, 7H), 2.19-2.13 (m, 2H), 1.48-1.34 (m, 3H).

### Example 309: 2-([3,3-difluoro-1-[(2S)-2-hydroxypropanoyl]piperidin-4-yl]oxy)-5-[2-([3-methoxy-5-[4-(oxetan-3-yl)piperazin-1-yl]pyridin-2-yl]amino)pyrimidin-4-yl]benzonitrile

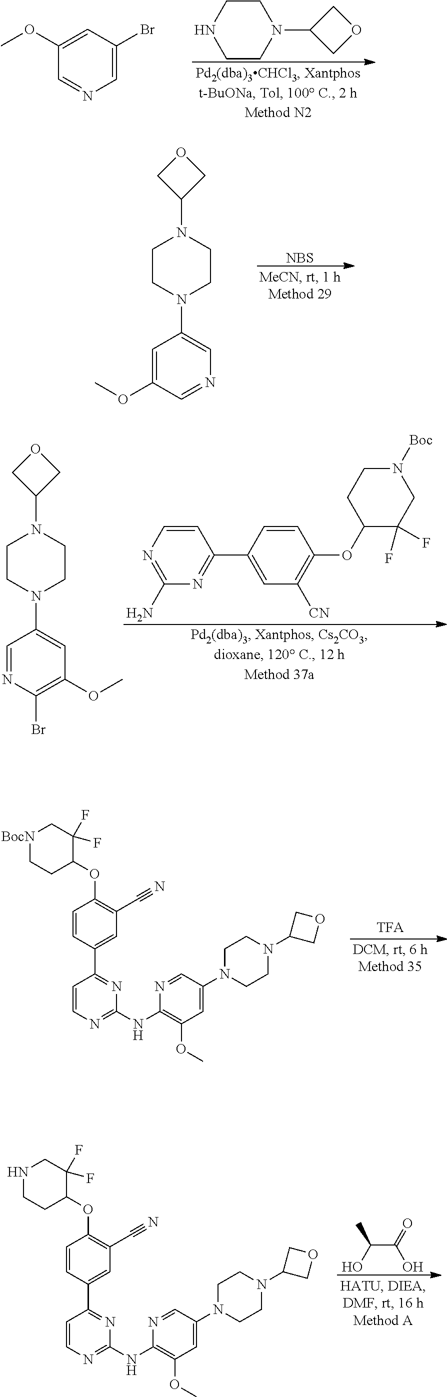

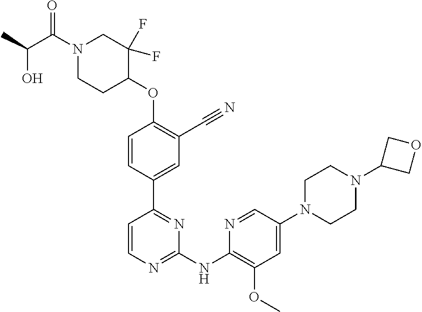 [SMILES: COc1cc(N2CCN(C3COC3)CC2)cnc1Nc1nccc(-c2ccc(OC3CCN(C(=O)[C@H](C)O)CC3(F)F)c(C#N)c2)n1]

2-[(3,3-difluoropiperidin-4-yl)oxy]-5-[2-([3-methoxy-5-[4-(oxetan-3-yl)piperazin-1-yl]pyridin-2-yl]amino)pyrimidin-4-yl]benzonitrile: The title compound was prepared from 3-bromo-5-methoxypyridine, 1-(oxetan-3-yl)piperazine, and tert-butyl 4-(4-(2-aminopyrimidin-4-yl)-2-cyanophenoxy)-3,3-difluoropiperidine-1-carboxylate using Method N2, 29,37a and 35. The final product was purified by prep-HPLC under the following condition: column, Gemini-NX C18 AXAI Packed, 21.2×150 mm, 5 um; mobile phase, acetonitrile in water (with 10 mmol/L NH₄HCO₃ and 0.1% NH₃.H₂O), 18% to 45% gradient in 8 min; detector, UV 254 nm. 2-[(3,3-difluoropiperidin-4-yl)oxy]-5-[2-([3-methoxy-5-[4-(oxetan-3-yl)piperazin-1-yl]pyridin-2-yl]amino)pyrimidin-4-yl]benzonitrile was obtained as a light yellow solid (9 mg, 4.5% for 4 steps). HPLC: 97.9% purity, RT=3.05 min. MS: m/z=579.0 [M+H]⁺. ¹H NMR (300 MHz, DMSO-d₆, ppm) δ 8.90 (s, 1H), 8.45-8.26 (m, 3H), 7.67-7.50 (m, 2H), 7.38-7.29 (m, 1H), 7.09-7.01 (m, 1H), 5.20-5.09 (m, 1H), 4.64-4.42 (m, 4H), 3.73 (s, 3H), 3.53-3.38 (m, 1H), 3.29-3.19 (m, 4H), 3.18-3.08 (m, 1H), 3.02-2.57 (m, 3H), 2.47-2.37 (m, 4H), 2.10-1.72 (m, 2H).

2-([3,3-difluoro-1-[(2S)-2-hydroxypropanoyl]piperidin-4-yl]oxy)-5-[2-([3-methoxy-5-[4-(oxetan-3-yl)piperazin-1-yl]pyridin-2-yl]amino)pyrimidin-4-yl]benzonitrile: The title compound was prepared from 2-[(3,3-difluoropiperidin-4-yl)oxy]-5-[2-([3-methoxy-5-[4-(oxetan-3-yl)piperazin-1-yl]pyridin-2-yl]amino)pyrimidin-4-yl]benzonitrile and (S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 18% to 45% gradient in 8 min; detector, UV 254 nm. 2-([3,3-difluoro-1-[(2S)-2-hydroxypropanoyl]piperidin-4-yl]oxy)-5-[2-([3-methoxy-5-[4-(oxetan-3-yl)piperazin-1-yl]pyridin-2-yl]amino)pyrimidin-4-yl]benzonitrile was obtained as a light yellow solid (14 mg, 13%). HPLC: 90.7% purity, RT=3.62 min. MS: m/z=651.4 [M+H]⁺. ¹H NMR (300 MHz, DMSO-d₆, ppm) δ 8.91 (s, 1H), 8.47-8.29 (m, 3H), 7.67-7.54 (m, 2H), 7.35 (d, J=5.2 Hz, 1H), 7.05 (d, J=2.5 Hz, 1H), 5.35-5.31 (m, 1H), 5.26-5.15 (m, 1H), 4.65-4.40 (m, 5H), 4.35-3.77 (m, 3H), 3.73 (s, 3H), 3.68-3.38 (m, 2H), 3.28-3.19 (m, 4H), 2.47-2.37 (m, 4H), 2.23-1.78 (m, 2H), 1.20 (d, J=6.5 Hz, 3H).

### Example 310: 2-([3,3-difluoro-1-[(2S)-2-hydroxypropanoyl]piperidin-4-yl]oxy)-5-[2-([2-methyl-4-[4-(oxetan-3-yl)piperazin-1-yl]phenyl]amino)pyrimidin-4-yl]benzonitrile

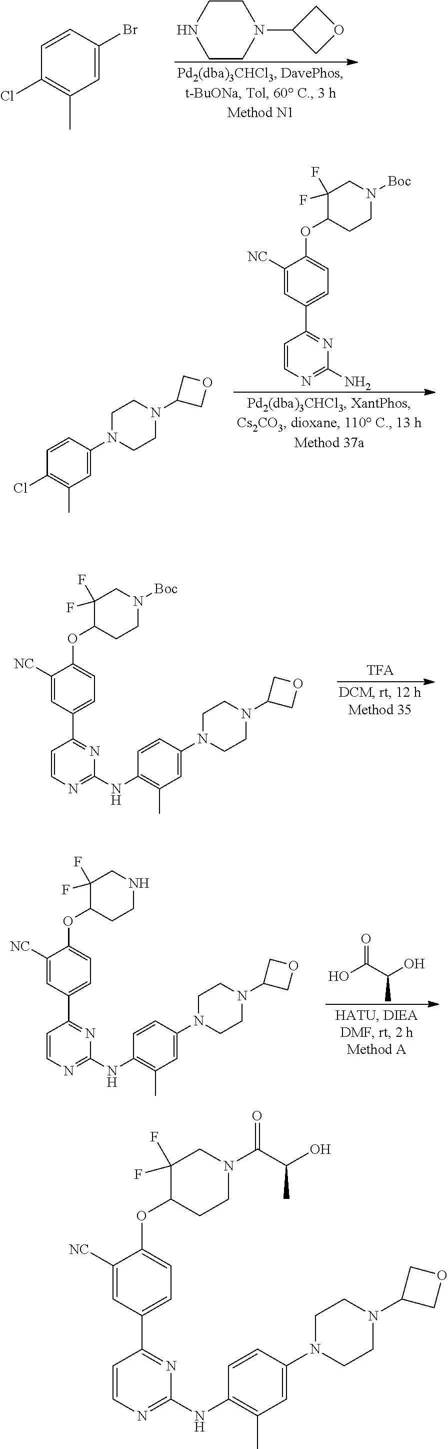

The title compound was prepared from 4-bromo-1-chloro-2-methylbenzene, 1-(oxetan-3-yl)piperazine, tert-butyl 4-(4-(2-aminopyrimidin-4-yl)-2-cyanophenoxy)-3,3-difluoropiperidine-1-carboxylate and (S)-2-hydroxypropanoic acid using Method N1, 37a, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 23% to 55% gradient in 8 min; detector, UV 254 nm. 2-([3,3-difluoro-1-[(2S)-2-hydroxypropanoyl]piperidin-4-yl]oxy)-5-[2-([2-methyl-4-[4-(oxetan-3-yl)piperazin-1-yl]phenyl]amino)pyrimidin-4-yl]benzonitrile was obtained as an yellow solid (27 mg, 3% for 4 steps). HPLC: 99.1% purity, RT=4.18 min. MS: m/z=634.3 [M+H]⁺. ¹H NMR (300 MHz, DMSO-d₆.ppm) δ 8.70 (s, 1H), 8.45 (d, J=2.2 Hz, 1H), 8.41-8.31 (m, 2H), 7.60 (d, J=9.1 Hz, 1H), 7.31 (d, J=5.2 Hz, 1H), 7.23 (d, J=8.6 Hz, 1H), 6.85-6.71 (m, 2H), 5.33 (s, 1H), 5.26-5.16 (m, 1H), 4.62-4.40 (m, 5H), 4.28-3.52 (m, 4H), 3.51-3.36 (m, 1H), 3.17-3.08 (m, 4H), 2.44-2.35 (m, 4H), 2.16 (s, 3H), 2.03-1.78 (m, 1H), 1.20 (d, 7=6.5 Hz, 3H).

### Example 311: 2-([3,3-difluoro-1-[(2S)-2-hydroxypropanoyl]piperidin-4-yl]oxy)-5-[2-([3-methoxy-4-[1-(oxetan-3-yl)piperidin-4-yl]phenyl]amino)pyrimidin-4-yl]benzonitrile

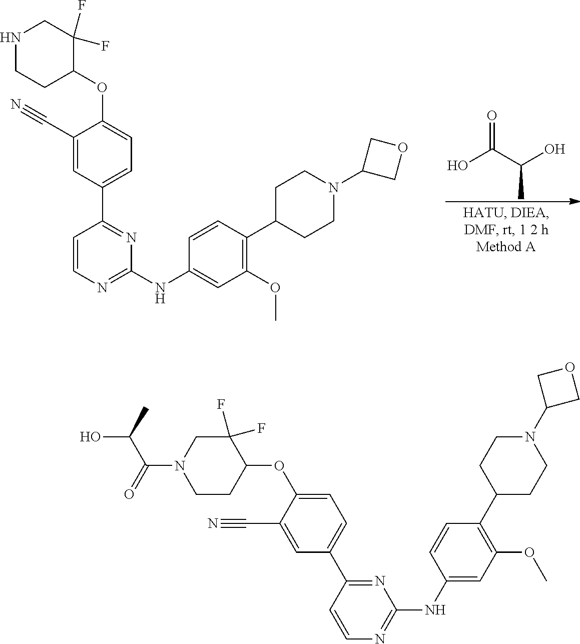

The title compound was prepared from 2-[(3,3-difluoropiperidin-4-yl)oxy]-5-[2-([3-methoxy-4-[1-(oxetan-3-yl)piperidin-4-yl]phenyl]amino)pyrimidin-4-yl]benzonitrile and (2S)-2-hydroxypropanoic acid using Method A. The final product was purified by prep-HPLC under the following condition: column, Atlantis HILIC OBD C18 Column, 150×19 mm, 5 um; mobile phase, acetonitrile in water (with 10 mmol/L NR₄HCO₃ and 0.1% NH₃.H₂O), 45% to 75% gradient in 8 min; detector, UV 254 nm. 2-([3,3-difluoro-1-[(2S)-2-hydroxypropanoyl]piperidin-4-yl]oxy)-5-[2-([3-methoxy-4-[1-(oxetan-3-yl)piperidin-4-yl]phenyl]amino)pyrimidin-4-yl]benzonitrile was obtained as a light yellow solid (18 mg, 17%). HPLC: 96.0% purity, RT=4.77 min. MS: m/z=649.2 [M+H]⁺. ¹H NMR (300 MHz, DMSO-d₆, ppm) δ 9.65 (s, 1H), 8.68-8.46 (m, 3H), 7.75-7.60 (m, 2H), 7.51-7.42 (m, 1H), 7.30-7.18 (m, 1H), 7.14-7.05 (m, 1H), 5.37 (br s, 1H), 5.29-5.16 (m, 1H), 4.61-4.34 (m, 5H), 4.29-3.93 (m, 3H), 3.81 (s, 3H), 3.71-3.53 (m, 1H), 3.44-3.35 (m, 1H), 2.92-2.67 (m, 3H), 2.23-1.93 (m, 2H), 1.90-1.74 (m, 2H), 1.74-1.54 (m, 2H), 1.20 (d, J=6.4 Hz, 3H).

### Example 312: 2-(2,7-Diaza-spiro[3.5]non-2-yl)-5-{2-[6-methoxy-5-(4-oxetan-3-yl-piperazin-1-yl)-pyridin-2-ylamino]-pyrimidin-4-yl}-benzonitrile

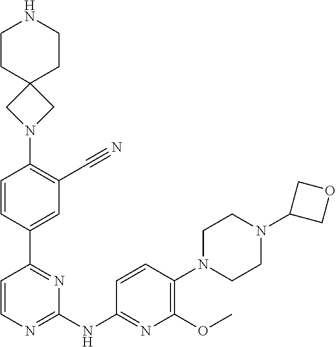 [SMILES: COc1nc(Nc2nccc(-c3ccc(N4CC5(CCNCC5)C4)c(C#N)c3)n2)ccc1N1CCN(C2COC2)CC1]

The title compound (350 mg) was synthesized using 2-(2-Cyano-4-{2-[6-methoxy-5-(4-oxetan-3-yl-piperazin-1-yl)-pyridin-2-ylamino]-pyrimidin-4-yl}-phenyl)-2,7-diaza-spiro[3.5]nonane-7-carboxylic acid tert-butyl ester (500 mg) and TFA (4 mL) using Method 17 in 82% yield, m/z: 568 (M+H). ¹H NMR (DMSO-d₆): 9.20 (1H), 8.44 (1H), 8.27 (1H), 7.74 (1H), 7.42 (1H), 7.27 (1H), 6.67 (1H), 4.58 (2H), 4.48 (2H), 3.91 (3H), 3.89 (3H), 349 (1H), 3.17 (1H), 2.99 (4H), 2.64 (3H), 2.41 (4H), 1.68 (3H).

### Example 313: 2-(2,7-Diaza-spiro[3.5]non-7-yl)-5-{2-[6-methoxy-5-(4-oxetan-3-yl-piperazin-1-yl)-pyridin-2-ylamino]-pyrimidin-4-yl}-benzonitrile

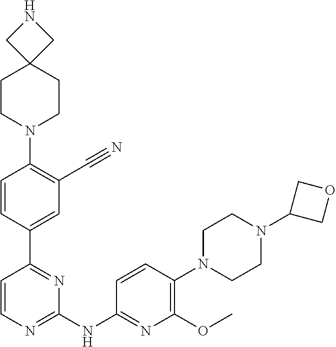 [SMILES: COc1nc(Nc2nccc(-c3ccc(N4CCC5(CC4)CNC5)c(C#N)c3)n2)ccc1N1CCN(C2COC2)CC1]

The title compound (130 mg) was synthesized using 7-(2-Cyano-4-{2-[6-methoxy-5-(4-oxetan-3-yl-piperazin-1-yl)-pyridin-2-ylamino]-pyrimidin-4-yl}-phenyl)-2,7-diaza-spiro[3.5]nonane-2-carboxylic acid tert-butyl ester (200 mg) and TFA (4 mL) using Method 17 in 73% yield, m/z: 568 (M+H). ¹H NMR (DMSO-d₆): 9.30 (1H), 8.56 (1H), 8.50 (1H), 8.35 (1H), 7.74 (1H), 7.42 (1H), 7.27 (2H), 4.58 (2H), 4.48 (2H), 3.91 (3H), 3.89 (3H), 349 (1H), 3.17 (1H), 2.99 (4H), 2.64 (3H), 2.41 (4H), 1.86 (2H), 1.79 (2H).

### Example 314.2-[7-((S)-2-Hydroxy-propionyl)-2,7-diaza-spiro[3.5]non-2-yl]-5-{2-[6-methoxy-5-(4-oxetan-3-yl-piperazin-1-yl)-pyridin-2-ylamino]-pyrimidin-4-yl}-benzonitrile

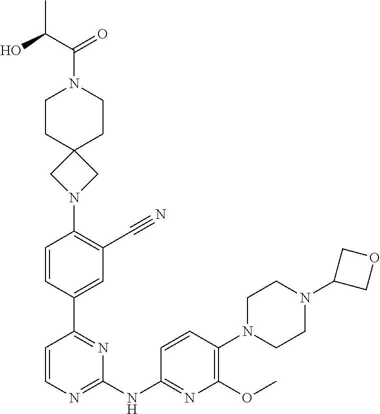 [SMILES: COc1nc(Nc2nccc(-c3ccc(N4CC5(CCN(C(=O)[C@H](C)O)CC5)C4)c(C#N)c3)n2)ccc1N1CCN(C2COC2)CC1]

The title compound (16.9 mg) was synthesized using 2-(2,7-Diaza-spiro[3.5]non-2-yl)-5-{2-[6-methoxy-5-(4-oxetan-3-yl-piperazin-1-yl)-pyridin-2-ylamino]-pyrimidin-4-yl}-benzonitrile (100 mg) and (S)-2-Hydroxy-propionic acid (31.40 mg) using Method A in 12% yield, m/z: 640 (M+H). ¹H NMR (DMSO-d₆): 9.19 (1H), 8.48 (1H), 8.37 (1H), 8.25 (1H), 7.74 (1H), 7.40 (1H), 7.27 (1H), 6.70 (1H), 4.82 (1H), 4.58 (2H), 4.48 (2H), 3.91 (3H), 3.89 (3H), 349 (1H), 3.17 (1H), 2.99 (4H), 2.64 (3H), 2.41 (4H), 1.86 (2H), 1.79 (2H), 1.71 (3H).

### Example 315: 2-[7-((S)-2,3-Dihydroxy-propionyl)-2,7-diaza-spiro[3.5]non-2-yl]-5-{2-[6-methoxy-5-(4-oxetan-3-yl-piperazin-1-yl)-pyridin-2-ylamino]-pyrimidin-4-yl}-benzonitrile

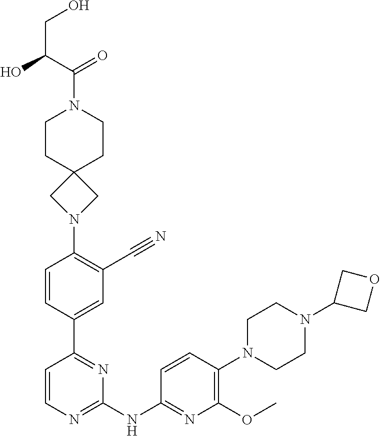 [SMILES: COc1nc(Nc2nccc(-c3ccc(N4CC5(CCN(C(=O)[C@@H](O)CO)CC5)C4)c(C#N)c3)n2)ccc1N1CCN(C2COC2)CC1]

The title compound (40.4 mg) was synthesized using 2-(2,7-Diaza-spiro[3.5]non-2-yl)-5-{2-[6-methoxy-5-(4-oxetan-3-yl-piperazin-1-yl)-pyridin-2-ylamino]-pyrimidin-4-yl}-benzonitrile (100 mg) and (S)-2,4-Dihydroxy-butyric acid (42.40 mg) using Method A in 35% yield, m/z: 656 (M+H). ¹H NMR (DMSO-d₆): 9.19 (1H), 8.48 (1H), 8.37 (1H), 8.25 (1H), 7.74 (1H), 7.40 (1H), 7.27 (1H), 6.70 (1H), 4.82 (1H), 4.58 (2H), 4.48 (2H), 4.03 (2H), 3.91 (3H), 3.89 (3H), 349 (1H), 3.17 (1H), 2.99 (4H), 2.64 (3H), 2.41 (4H), 1.86 (2H), 1.79 (2H).

### Example 316: 2-[2-((S)-2-Hydroxy-propionyl)-2,7-diaza-spiro[3.5]non-7-yl]-5-{2-[6-methoxy-5-(4-oxetan-3-yl-piperazin-1-yl)-pyridin-2-ylamino]-pyrimidin-4-yl}-benzonitrile

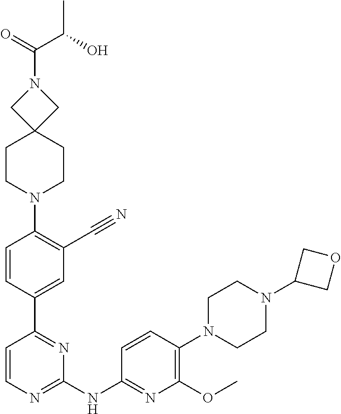 [SMILES: COc1nc(Nc2nccc(-c3ccc(N4CCC5(CC4)CN(C(=O)[C@H](C)O)C5)c(C#N)c3)n2)ccc1N1CCN(C2COC2)CC1]

The title compound (2.1 mg) was synthesized using 2-(2,7-Diaza-spiro[3.5]non-7-yl)-5-{2-[6-methoxy-5-(4-oxetan-3-yl-piperazin-1-yl)-pyridin-2-ylamino]-pyrimidin-4-yl}-benzonitrile (60 mg) and (S)-2-Hydroxy-propionic acid (19.40 mg) using Method A in 3% yield, m/z: 640 (M+H). ¹H NMR (DMSO-d₆): 9.19 (1H), 8.48 (1H), 8.37 (1H), 8.25 (1H), 7.74 (1H), 7.40 (1H), 7.27 (1H), 6.70 (1H), 4.82 (1H), 4.58 (2H), 4.48 (2H), 3.91 (3H), 3.89 (3H), 349 (1H), 3.17 (1H), 2.99 (4H), 2.64 (3H), 2.41 (4H), 1.86 (2H), 1.79 (2H), 1.71 (3H).

### Example 317: 2-[2-((S)-2,3-Dihydroxy-propionyl)-2,7-diaza-spiro[3.5]non-7-yl]-5-{2-[6-methoxy-5-(4-oxetan-3-yl-piperazin-1-yl)-pyridin-2-ylamino]-pyrimidin-4-yl}-benzonitrile

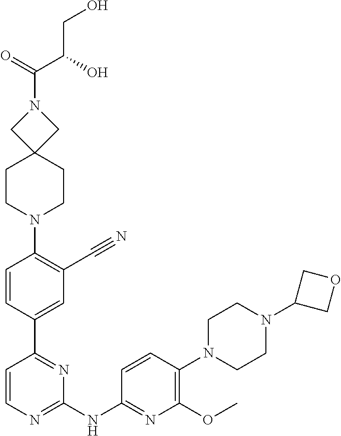 [SMILES: COc1nc(Nc2nccc(-c3ccc(N4CCC5(CC4)CN(C(=O)[C@@H](O)CO)C5)c(C#N)c3)n2)ccc1N1CCN(C2COC2)CC1]

The title compound (1.1 mg) was synthesized using 2-(2,7-Diaza-spiro[3.5]non-7-yl)-5-{2-[6-methoxy-5-(4-oxetan-3-yl-piperazin-1-yl)-pyridin-2-ylamino]-pyrimidin-4-yl}-benzonitrile (60 mg) and (S)-2,4-Dihydroxy-butyric acid (22.40 mg) using Method A in 2% yield, m/z: 656 (M+H). ¹H NMR (DMSO-d₆): 9.19 (1H), 8.48 (1H), 8.37 (1H), 8.25 (1H), 7.74 (1H), 7.40 (1H), 7.27 (1H), 6.70 (1H), 4.82 (1H), 4.58 (2H), 4.48 (2H), 4.03 (2H), 3.91 (3H), 3.89 (3H), 349 (1H), 3.17 (1H), 2.99 (4H), 2.64 (3H), 2.41 (4H), 1.86 (2H), 1.79 (2H).

### Example 318: 2-([1-[(2S)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile

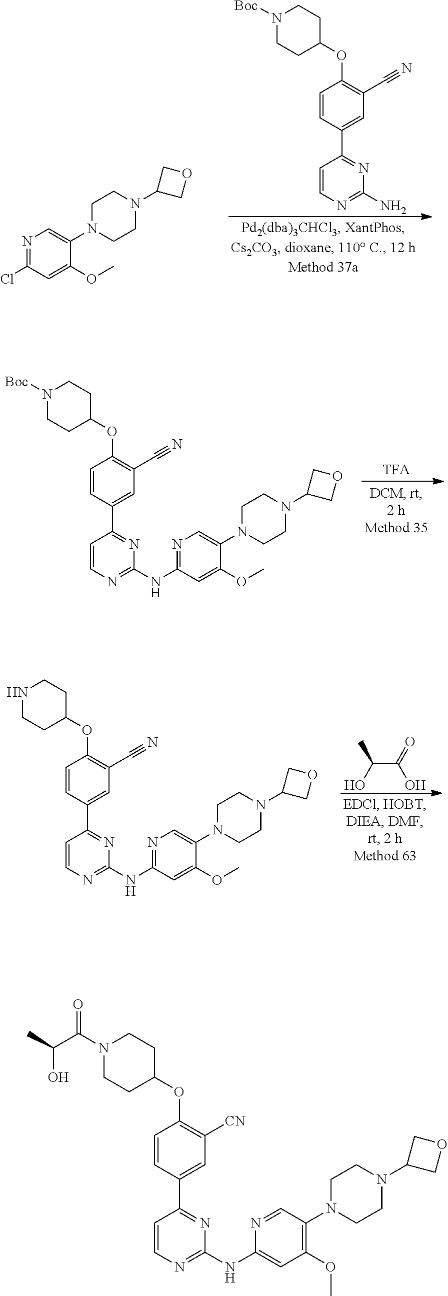

The title compound was prepared from 1-(6-chloro-4-methoxypyridin-3-yl)-4-(oxetan-3-yl)piperazine, tert-butyl 4-(4-(2-aminopyrimidin-4-yl)-2-cyanophenoxy)piperidine-1-carboxylate and (S)-2-hydroxypropanoic acid using Method 37a, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 30% to 60% gradient in 8 min; detector, UV 254 nm. 2-([1-[(2S)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile was obtained as an yellow solid (16 mg, 7% for 3 step). HPLC: 99.9% purity, RT=2.88 min. MS: m/z=615.3 [M+H]⁺. ¹H NMR (300 MHz, DMSO-d₆, ppm) δ9.68 (s, 1H), 8.62-8.55 (m, 2H), 8.46 (dd, J=9.0, 2.3 Hz, 1H), 8.07 (s, 1H), 7.77 (s, 1H), 7.58-7.48 (m, 2H), 5.08-4.89 (m, 2H), 4.63-4.37 (m, 5H), 3.94 (s, 3H), 3.87-3.62 (m, 2H), 3.58-3.37 (m, 3H), 3.03-2.97 (m, 4H), 2.42-2.36 (m, 4H), 2.01-1.95 (m, 2H), 1.82-1.48 (m, 2H), 1.19 (d, J=6.5 Hz, 3H).

### Example 319: 2-([1-[(2R)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile

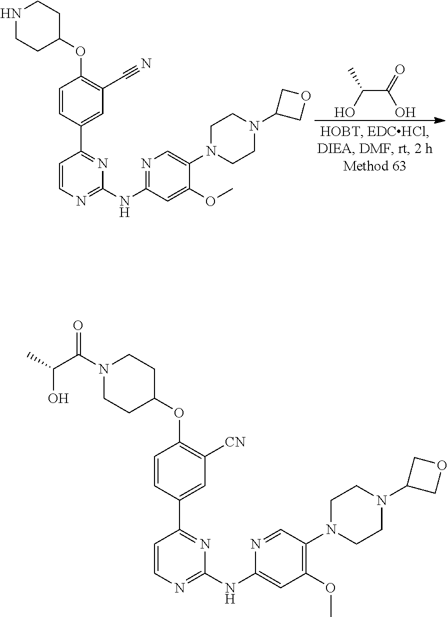

The title compound was prepared from 5-(2-(4-methoxy-5-(4-(oxetan-3-yl)piperazin-1-yl)pyridin-2-ylamino)pyrimidin-4-yl)-2-(piperidin-4-yloxy)benzonitrile and (R)-2-hydroxypropanoic acid using Method 63. The final product was purified by prep-HPLC under the following conditions: column, XBridge Prep OBD C18 Column, 150×30 mm, 5 um; mobile phase, acetonitrile in water (with 10 mmol/L NR₄HCO₃ and 0.1% NH₃.H₂O), 30% to 60% gradient in 8 min; detector, UV 254 nm. 2-([1-[(2R)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile was obtained as an yellow solid (26 mg, 22%). HPLC: 96.2% purity, RT=5.15 min. MS: m/z=615.4 [M+H]⁺. ¹H NMR (400 MHz, DMSO-d₆, ppm) δ 9.75 (s, 1H), 8.63-8.58 (m, 2H), 8.50-8.43 (m, 1H), 8.09 (s, 1H), 7.81 (s, 1H), 7.58-7.50 (m, 2H), 5.07-4.90 (m, 2H), 4.62-4.42 (m, 5H), 3.95 (s, 3H), 3.87-3.64 (m, 2H), 3.63-3.39 (m, 3H), 3.04-2.99 (m, 4H), 2.43-2.38 (m, 4H), 2.10-1.87 (m, 2H), 1.87-1.56 (m, 2H), 1.20 (d, J=6.5 Hz, 3H).

### Example 320: 2-([1-[(2S)-2-hydroxypropanoyl]piperidin-4-yl]oxy)-5-[2-([5-methoxy-6-[4-(oxetan-3-yl)piperazin-1-yl]pyridin-3-yl]amino)pyrimidin-4-yl]benzonitrile

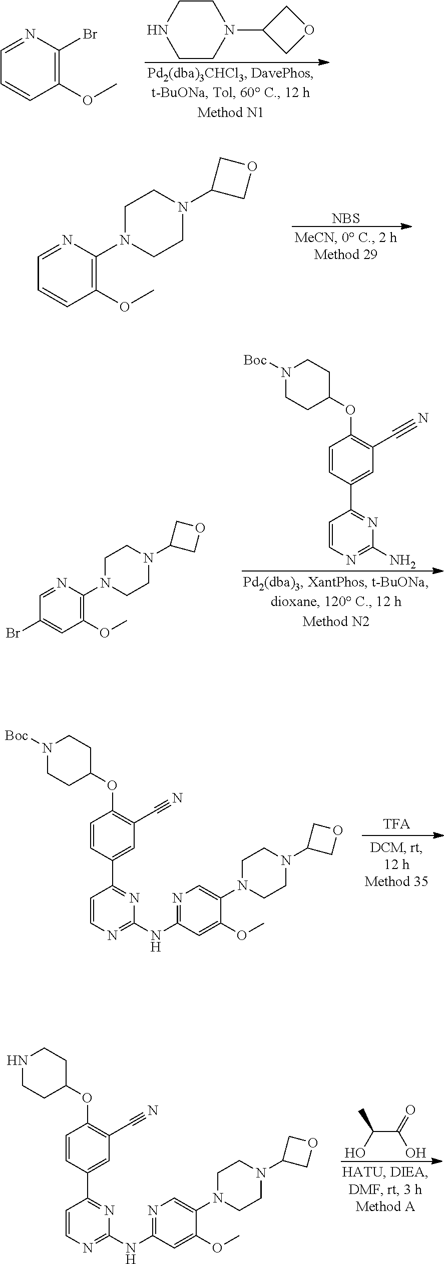

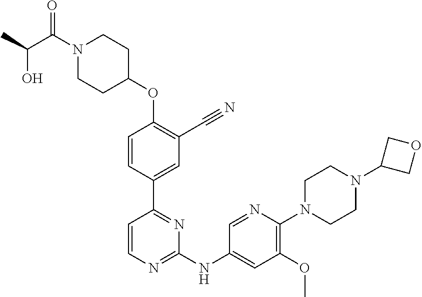 [SMILES: COc1cc(Nc2nccc(-c3ccc(OC4CCN(C(=O)[C@H](C)O)CC4)c(C#N)c3)n2)cnc1N1CCN(C2COC2)CC1]

The title compound was prepared from 2-bromo-3-methoxypyridine, 1-(oxetan-3-yl)piperazine, NBS, tert-butyl 4-(4-(2-aminopyrimidin-4-yl)-2-cyanophenoxy)piperidine-1-carboxylate and (S)-2-hydroxypropanoic acid using Method N1, 29, N2, 35 and A. The final product was purified by prep-HPLC under the following conditions: column, Xselect CSH OBD C18 Column, 150×30 mm, 5 um; mobile phase, acetonitrile in water (with 10 mmol/L NH₄HCO₃), 30% to 35% gradient in 8 min; detector, UV 254 nm. 2-([1-[(2S)-2-hydroxypropanoyl]piperidin-4-yl]oxy)-5-[2-([5-methoxy-6-[4-(oxetan-3-yl)piperazin-1-yl]pyridin-3-yl]amino)pyrimidin-4-yl]benzonitrile was obtained as an yellow solid (28 mg, 1% for 5 step). HPLC: 98.4% purity, RT=7.34 min. MS: m/z=615.3 [M+H]⁺. ¹H NMR (300 MHz, DMSO-d₆. ppm) δ9.58 (s, 1H), 8.55-8.47 (m, 2H), 8.41 (dd, J=9.0, 2.3 Hz, 1H), 8.15 (d, 7=2.1 Hz, 1H), 7.86 (d, J=2.1 Hz, 1H), 7.53 (d, J=9.1 Hz, 1H), 7.42 (d, J=5.3 Hz, 1H), 5.08-4.77 (m, 2H), 4.61-4.36 (m, 5H), 3.83 (s, 3H), 3.78-3.63 (m, 2H), 3.54-3.36 (m, 3H), 3.27-3.18 (m, 4H), 2.42-2.32 (m, 4H), 2.01-1.95 (m, 2H), 1.74-1.68 (m, 2H), 1.19 (d, J=6.5 Hz, 3H).

### Example 321:2-([1-[(2R)-2-hydroxypropanoyl]piperidin-4-yl]oxy)-5-[2-([5-methoxy-6-[4-(oxetan-3-yl)piperazin-1-yl]pyridin-3-yl]amino)pyrimidin-4-yl]benzonitrile

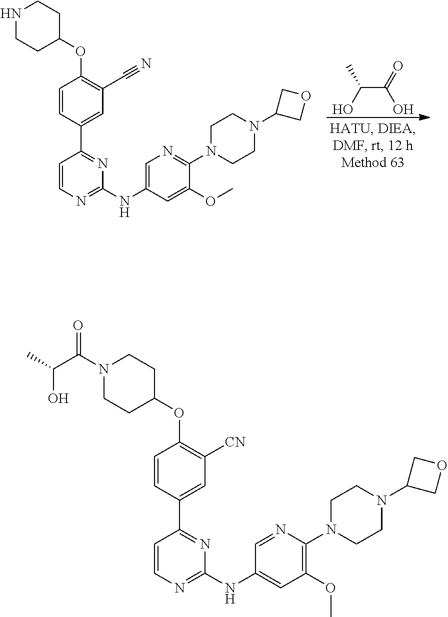

The title compound was prepared from 5-(2-(5-methoxy-6-(4-(oxetan-3-yl)piperazin-1-yl)pyridin-3-ylamino)pyrimidin-4-yl)-2-(piperidin-4-yloxy)benzonitrile and (R)-2-hydroxypropanoic acid using Method A. The final product was purified by prep-HPLC under the following conditions: column, Xselect CSH OBD C18 Column, 150×30 mm, 5 um; mobile phase, acetonitrile in water (with 10 mmol/L NH₄HCO₃), 30% to 35% gradient in 8 min; detector, UV 254 nm. 2-([1-[(2R)-2-hydroxypropanoyl]piperidin-4-yl]oxy)-5-[2-([5-methoxy-6-[4-(oxetan-3-yl)piperazin-1-yl]pyridin-3-yl]amino)pyrimidin-4-yl]benzonitrile was obtained as an yellow solid (32 mg, 23%). HPLC: 99.1% purity, RT=3.17 min. MS: m/z=615.3 [M+H]⁺. ¹H NMR (300 MHz, DMSO-d₆, ppm) δ9.58 (s, 1H), 8.55-8.47 (m, 2H), 8.41 (dd, J=9.0, 2.3 Hz, 1H), 8.15 (d, 7=2.1 Hz, 1H), 7.86 (d, J=2.2 Hz, 1H), 7.54 (d, J=9.1 Hz, 1H), 7.42 (d, J=5.3 Hz, 1H), 5.05-4.86 (m, 2H), 4.60-4.41 (m, 5H), 3.82 (s, 3H), 3.79-3.62 (m, 2H), 3.58-3.36 (m, 3H), 3.27-3.18 (m, 4H), 2.41-2.32 (m, 4H), 2.01-1.95 (m, 2H), 1.73-1.67 (m, 2H), 1.19 (d, J=6.5 Hz, 3H).

### Example 322: 2-([33-difluoro-1-[(2S)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile

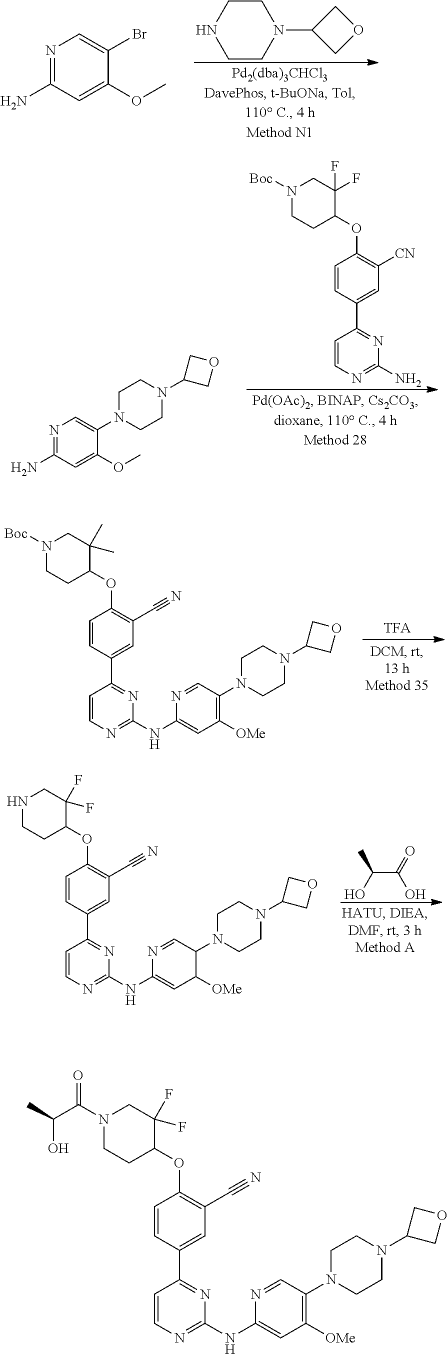

2-[(3,3-difluoropiperidin-4-yl)oxy]-5-[2-([4-methoxy-5-[4-(oxetan-3-yl)piperazin-1-yl]pyridin-2-yl]amino)pyrimidin-4-yl]benzonitrile: The title compound was prepared from 5-bromo-4-methoxypyridin-2-amine, 1-(oxetan-3-yl)piperazine and tert-butyl 4-(4-(2-aminopyrimidin-4-yl)-2-cyanophenoxy)-3,3-difluoropiperidine-1-carboxylate using Method N1, 28 and 35. The final product was purified by prep-HPLC under the following conditions: column, XBridge Prep OBD C18 Column, 150×30 mm, 5 um; mobile phase, acetonitrile in water (with 10 mmol/L NH₄HCO₃), 30% to 60% gradient in 8 min; detector, UV 254 nm. 2-[(3,3-difluoropiperidin-4-yl)oxy]-5-[2-([4-methoxy-5-[4-(oxetan-3-yl)piperazin-1-yl]pyridin-2-yl]amino)pyrimidin-4-yl]benzonitrile was obtained as an yellow solid (6 mg, 14% for 3 step). HPLC: 97.2% purity, RT=2.20 min. MS: m/z=579.2 [M+H]⁺. ¹H NMR (300 MHz, DMSO-d₆, ppm) δ9.75 (s, 1H), 8.64-8.56 (m, 2H), 8.52-8.43 (m, 1H), 8.06 (s, 1H), 7.78 (s, 1H), 7.66-7.57 (m, 1H), 7.57-7.50 (m, 1H), 5.23 (br s, 1H), 4.68-4.41 (m, 4H), 3.94 (s, 3H), 3.50-3.44 (m, 2H), 3.03-2.65 (m, 8H), 2.43-2.37 (m, 4H), 2.17-1.74 (m, 2H).

2-([3,3-difluoro-1-[(2S)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile: The title compound was prepared from 5-bromo-4-methoxypyridin-2-amine, 1-(oxetan-3-yl)piperazine, tert-butyl 4-(4-(2-aminopyrimidin-4-yl)-2-cyanophenoxy)-3,3-difluoropiperidine-1-carboxylate and (S)-2-hydroxypropanoic acid using Method N1, 28,35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃ and 0.1% NH₃.H₂O), 22% to 49% gradient in 8 min; detector, UV 254 nm. 2-([3,3-difluoro-1-[(2S)-2-hydroxypropanoyl]piperidin-4-yl]oxy)-5-[2-([4-methoxy-5-[4-(oxetan-3-yl)piperazin-1-yl]pyridin-2-yl]amino)pyrimidin-4-yl]benzonitrile was obtained as an yellow solid (25 mg, 9% for 4 step). HPLC: 98.3% purity, RT=2.78 min. MS: m/z=651.2 [M+H]⁺. ¹H NMR (300 MHz, DMSO-d₆, ppm) δ 9.93 (s, 1H), 8.69-8.61 (m, 2H), 8.53 (dd, J=9.1, 2.3 Hz, 1H), 8.02 (s, 1H), 7.81 (s, 1H), 7.62-7.58 (m, 2H), 5.47-5.22 (m, 2H), 4.70-4.35 (m, 4H), 4.26-3.94 (m, 5H), 3.93-3.47 (m, 4H), 3.05-3.03 (m, 4H), 2.45-2.21 (m, 4H), 2.19-1.89 (m, 2H), 1.22 (d, J=6.5 Hz, 3H).

### Example 323: 2-([3,3-difluoro-1-[(2S)-2-hydroxypropanoyl]piperidin-4-yl]oxy)-5-[2-([5-methoxy-6-[4-(oxetan-3-yl)piperazin-1-yl]pyridin-3-yl]amino)pyrimidin-4-yl]benzonitrile

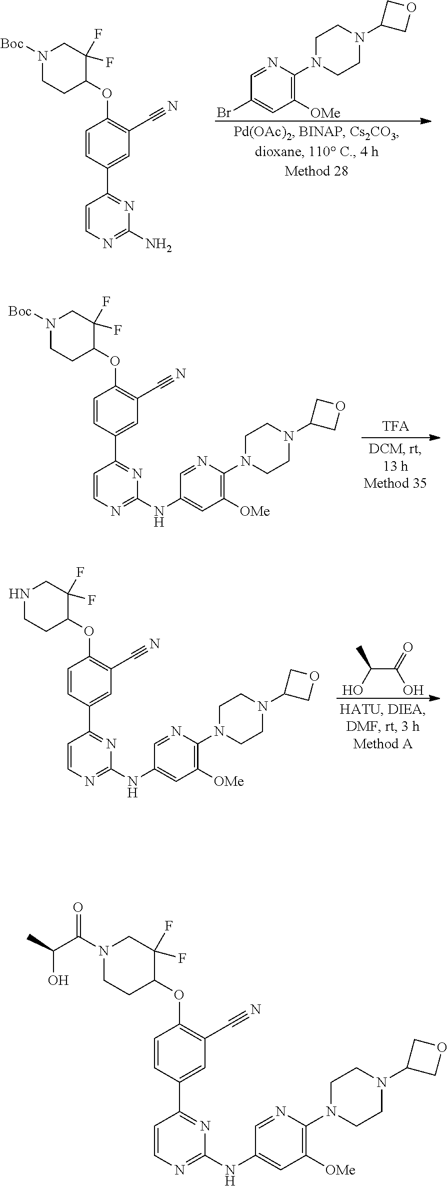

The title compound was prepared from 2-bromo-3-methoxypyridine, 1-(oxetan-3-yl)piperazine, NBS, tert-butyl 4-(4-(2-aminopyrimidin-4-yl)-2-cyanophenoxy)piperidine-1-carboxylate and (S)-2-hydroxypropanoic acid using Method N1, 29, N2, 35 and A. The final product was purified by prep-HPLC under the following conditions: column, XBridge Prep OBD C18 Column, 150×30 mm, 5 um; mobile phase, acetonitrile in water (with 10 mmol/L NR₄HCO₃), 20% to 50% gradient in 8 min; detector, UV 254 nm. 2-([3,3-difluoro-1-[(2S)-2-hydroxypropanoyl]piperidin-4-yl]oxy)-5-[2-([5-methoxy-6-[4-(oxetan-3-yl)piperazin-1-yl]pyridin-3-yl]amino)pyrimidin-4-yl]benzonitrile was obtained as an yellow solid (35 mg, 9% for 3 steps). HPLC: 97.9% purity, RT=3.58 min. MS: m/z=651.2 [M+H]1H NMR (300 MHz, DMSO-d6, ppm) δ 9.63 (s, 1H), 8.59-8.39 (m, 3H), 8.15 (d, J=2.1 Hz, 1H), 7.87 (s, 1H), 7.66 (d, J=9.1 Hz, 1H), 7.46 (d, J=5.3 Hz, 1H), 5.41-5.15 (m, 2H), 4.60-4.32 (m, 5H), 4.23-3.54 (m, 7H), 3.48-3.38 (m, 1H), 3.25-3.15 (m, 4H), 2.37-2.30 (m, 4H), 2.21-1.78 (m, 2H), 1.21 (d, J=6.5 Hz, 3H).

### Example 324: 6-([4-[3-cyano-4-([3,3-difluoro-1-[(2S)-2-hydroxypropanoyl]piperidin-4-yl]oxy)phenyl]pyrimidin-2-yl]amino)-2-methoxy-N,N-dimethylpyridine-3-carboxamide

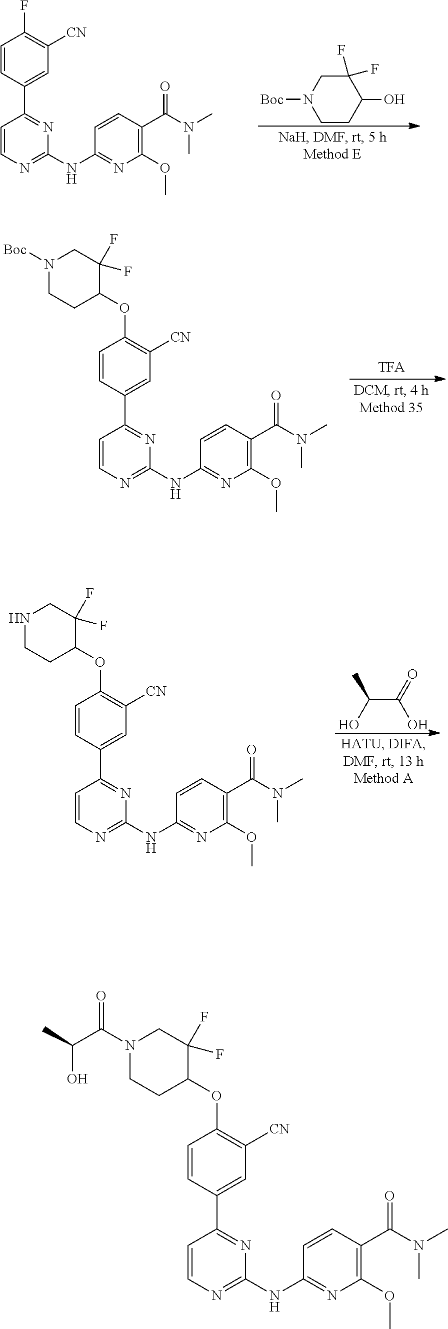

6-[(4-[3-cyano-4-[(3,3-difluoropiperidin-4-yl)oxy]phenyl]pyrimidin-2-yl)amino]-2-methoxy-N,N-dimethylpyridine-3-carboxamide: The title compound was prepared from 6-(4-(3-cyano-4-fluorophenyl)pyrimidin-2-ylamino)-2-methoxy-N,N-dimethylnicotinamide and tert-butyl 3,3-difluoro-4-hydroxypiperidine-1-carboxylate using Method E and 35. The final product was purified by prep-HPLC under the following conditions: column, XBridge Prep OBD C18 Column, 150×30 mm, 5 um; mobile phase, acetonitrile in water (with 10 mmol/L NR₄HCO₃), 30% to 60% gradient in 8 min; detector, UV 254 nm. 6-[(4-[3-cyano-4-[(3,3-difluoropiperidin-4-yl)oxy]phenyl]pyrimidin-2-yl)amino]-2-methoxy-N,N-dimethylpyridine-3-carboxamide was obtained as an yellow solid (420 mg, 27% for 2 steps). HPLC: 99.5% purity, RT=6.65 min. MS: m/z=510.2 [M+H]⁺. ¹H NMR (300 MHz, DMSO-d₆, ppm) δ9.87 (s, 1H), 8.69-8.58 (m, 2H), 8.55-8.46 (m, 1H), 7.94-7.85 (m, 1H), 7.68-7.59 (m, 3H), 5.25-5.21 (m, 1H), 3.91 (s, 3H), 3.18-3.10 (m, 1H), 3.03-2.79 (m, 8H), 2.71-2.61 (m, 1H), 2.09-1.83 (m, 2H).

6-([4-[3-cyano-4-([33-difluoro-1-[(2S)-2-hydroxypropanoyl]piperidin-4-yl]oxy)phenyl]pyrimidin-2-yl]amino)-2-methoxy-N,N-dimethylpyridine-3-carboxamide: The title compound was prepared from 6-(4-(3-cyano-4-fluorophenyl)pyrimidin-2-ylamino)-2-methoxy-N,N-dimethylnicotinamide, tert-butyl 3,3-difluoro-4-hydroxypiperidine-1-carboxylate and (S)-2-hydroxypropanoic acid using Method E, 35 and A. The final product was purified by prep-HPLC under the following conditions: column, XBridge Prep OBD C18 Column, 150×30 mm, 5 um; mobile phase, acetonitrile in water (with 10 mmol/L NH₄HCO₃), 25% to 48% gradient in 8 min; detector, UV 254 nm. 6-([4-[3-cyano-4-([3,3-difluoro-1-[(2S)-2-hydroxypropanoyl]piperidin-4-yl]oxy)phenyl]pyrimidin-2-yl]amino)-2-methoxy-N,N-dimethylpyridine-3-carboxamide was obtained as an yellow solid (27 mg, 7% for 3 steps). HPLC: 99.1% purity, RT=5.28 min. MS: m/z=581.8 [M+H]⁺. ¹H NMR (300 MHz, DMSO-d₆. ppm) δ 9.93 (s, 1H), 8.73-8.63 (m, 2H), 8.56 (dd, J=9.0, 2.3 Hz, 1H), 7.93 (d, J=8.1 Hz, 1H), 7.74-7.62 (m, 3H), 5.50-5.34 (m, 1H), 5.34-5.18 (m, 1H), 4.61-4.43 (m, 1H), 4.00-4.30 (m, 1H), 3.93 (s, 3H), 3.89-3.59 (m, 2H), 2.98 (s, 3H), 2.84 (s, 3H), 2.29-1.84 (m, 2H), 1.21 (d, J=6.3 Hz, 3H).

### Example 325: 6-([4-[3-cyano-4-([3,3-difluoro-1-[(2R)-2-hydroxypropanoyl]piperidin-4-yl]oxy)phenyl]pyrimidin-2-yl]amino)-2-methoxy-N,N-dimethylpyridine-3-carboxamide

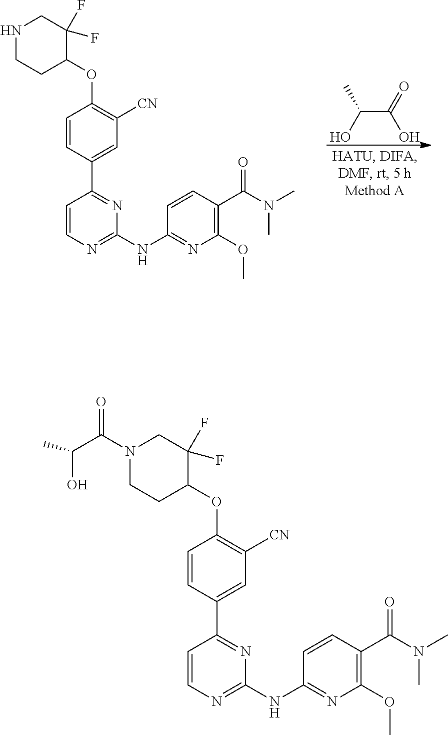

The title compound was prepared from 6-(4-(3-cyano-4-(3,3-difluoropiperidin-4-yloxy)phenyl)pyrimidin-2-ylamino)-2-methoxy-N,N-dimethylnicotinamide and (R)-2-hydroxypropanoic acid using Method A. The final product was purified by prep-HPLC under the following conditions: column, XBridge Prep OBD C18 Column, 150×30 mm, 5 um; mobile phase, acetonitrile in water (with 10 mmol/L NH₄HCO₃), 35% to 62% gradient in 8 min; detector, UV 254 nm. 6-([4-[3-cyano-4-([3,3-difluoro-1-[(2R)-2-hydroxypropanoyl]piperidin-4-yl]oxy)phenyl]pyrimidin-2-yl]amino)-2-methoxy-N,N-dimethylpyridine-3-carboxamide was obtained as an yellow solid (31 mg, 15%). HPLC: 99.1% purity, RT=5.28 min. MS: m/z=581.8 [M+H]⁺. ¹H NMR (300 MHz, DMSO-d₆, ppm) δ 9.92 (s, 1H), 8.67-8.63 (m, 2H), 8.55 (d, J=9.0 Hz, 1H), 7.92 (d, J=8.0 Hz, 1H), 7.76-7.55 (m, 3H), 5.50-5.33 (m, 1H), 5.31-5.18 (m, 1H), 4.59-4.42 (m, 1H), 4.29-3.96 (m, 2H), 3.92 (s, 3H), 3.86-3.55 (m, 2H), 2.97 (s, 3H), 2.83 (s, 3H), 2.21-1.87 (m, 2H), 1.22 (d, J=6.3 Hz, 3H).

### Example 326: 2-[[3,3-difluoro-1-(2-hydroxyacetyl)piperidin-4-yl]oxy]-5-[2-[(2-methoxypyridin-4-yl)amino]pyrimidin-4-yl]benzonitrile

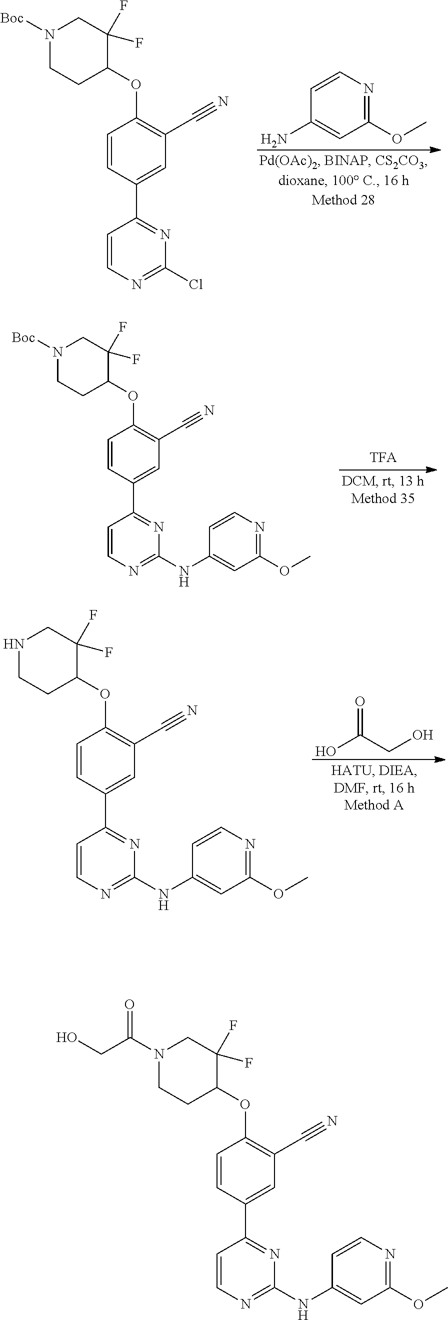

2-[(3,3-difluoropiperidin-4-yl)oxy]-5-[2-[(2-methoxypyridin-4-yl)amino]pyrimidin-4-yl]benzonitrile: The title compound was prepared from tert-butyl 4-[4-(2-chloropyrimidin-4-yl)-2-cyanophenoxy]-3,3-difluoropiperidine-1-carboxylate and 2-methoxypyridin-4-amine using Method 28 and 35. The final product was purified by prep-HPLC under the following condition: column, Atlantis HILIC OBD C18 Column, 150×19 mm, 5 um; mobile phase, acetonitrile in water (with 10 mmol/L NH₄HCO₃ and 0.1% NH₃.H₂O), 25% to 48% gradient in 8 min; detector, UV 254 nm. 2-[(3,3-difluoropiperidin-4-yl)oxy]-5-[2-[(2-methoxypyridin-4-yl)amino]pyrimidin-4-yl]benzonitrile was obtained as white solid (5 mg, 1.2% for 2 steps). HPLC: 98.8% purity, RT=2.59 min. MS: m/z=439.2 [M+H]⁺. ¹H NMR (300 MHz, DMSO-d₆, ppm) δ 10.16 (s, 1H), 8.71-8.63 (m, 1H), 8.62-8.55 (m, 1H), 8.53-8.43 (m, 1H), 8.03-7.94 (m, 1H), 7.72-7.60 (m, 2H), 7.48-7.41 (m, 1H), 7.36-7.26 (m, 1H), 5.31-5.17 (m, 1H), 3.84 (s, 3H), 3.19-3.12 (m, 1H), 3.06-2.81 (m, 2H), 2.76-2.69 (m, 1H), 2.58-2.51 (m, 1H), 2.13-1.74 (m, 2H).

2-[[3,3-difluoro-1-(2-hydroxyacetyl)piperidin-4-yl]oxy]-5-[2-[(2-methoxypyridin-4-yl)amino]pyrimidin-4-yl]benzonitrile: The title compound was prepared from 2-[(3,3-difluoropiperidin-4-yl)oxy]-5-[2-[(2-methoxypyridin-4-yl)amino]pyrimidin-4-yl]benzonitrile and 2-hydroxyacetic acid using Method A. The final product was purified by prep-HPLC under the following condition: column, XBridge Prep OBD C18 Column, 150×30 mm, 5 um; mobile phase, acetonitrile in water (with 10 mmol/L NH₁HCO₃ and 0.1% NH₃.H₂O), 30% to 50% gradient in 8 min; detector, UV 254 nm. 2-[[3,3-difluoro-1-(2-hydroxyacetyl)piperidin-4-yl]oxy]-5-[2-[(2-methoxypyridin-4-yl)amino]pyrimidin-4-yl]benzonitrile was obtained as an yellow solid (25 mg, 23%). HPLC: 97.1% purity, RT=5.67 min. MS: m/z=497.2 [M+H]⁺. ¹H NMR (300 MHz, DMSO-d₆, ppm) δ 10.14 (s, 1H), 8.69-8.54 (m, 2H), 8.53-8.43 (m, 1H), 8.01-7.92 (m, 1H), 7.73-7.58 (m, 2H), 7.46-7.39 (m, 1H), 7.33-7.24 (m, 1H), 5.45-5.31 (m, 1H), 4.93-4.87 (m, 1H), 4.25-3.96 (m, 3H), 3.95-3.81 (m, 1H), 3.81 (s, 3H), 3.68-3.43 (m, 2H), 2.26-1.73 (m, 2H).

### Example 327: 2-([3,3-difluoro-1-[(2S)-2-hydroxypropanoyl]piperidin-4-yl]oxy)-5-[2-[(2-methoxypyridin-4-yl)amino]pyrimidin-4-yl]benzonitrile

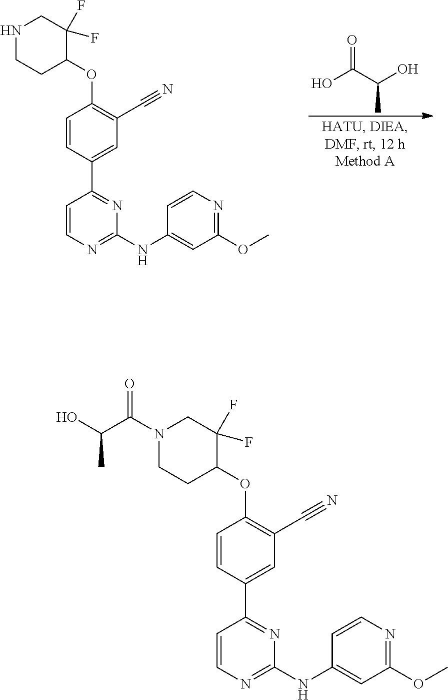

2-([3,3-difluoro-1-[(2S)-2-hydroxypropanoyl]piperidin-4-yl]oxy)-5-[2-[(2-methoxypyridin-4-yl)amino]pyrimidin-4-yl]benzonitrile: The title compound was prepared from 2-[(3,3-difluoropiperidin-4-yl)oxy]-5-[2-[(2-methoxypyridin-4-yl)amino]pyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 33% to 55% gradient in 8 min; detector, UV 254 nm. 2-([3,3-difluoro-1-[(2S)-2-hydroxypropanoyl] piperidin-4-yl]oxy)-5-[2-[(2-methoxypyridin-4-yl)amino]pyrimidin-4-yl]benzonitrile was obtained as white solid (26 mg, 25%). HPLC: 97.8% purity, RT=4.48 min. MS: m/z=511.2 [M+H]⁺. ¹H NMR (300 MHz, DMSO-d₆, ppm) δ 10.13 (s, 1H), 8.68-8.60 (m, 1H), 8.60-8.52 (m, 1H), 8.52-8.42 (m, 1H), 8.00-7.91 (m, 1H), 7.71-7.53 (m, 2H), 7.45-7.38 (m, 1H), 7.32-7.23 (m, 1H), 5.40-5.33 (m, 1H), 5.29-5.23 (m, 1H), 4.52-4.45 (m, 1H), 4.33-3.50 (m, 7H), 2.23-1.75 (m, 2H), 1.20 (d, J=6.5 Hz, 3H).

### Example 328: 2-([3,3-difluoro-1-[(2S)-2-hydroxypropanoyl]piperidin-4-yl]oxy)-5-[2-[(6-methoxypyridin-2-yl)amino]pyrimidin-4-yl]benzonitrile

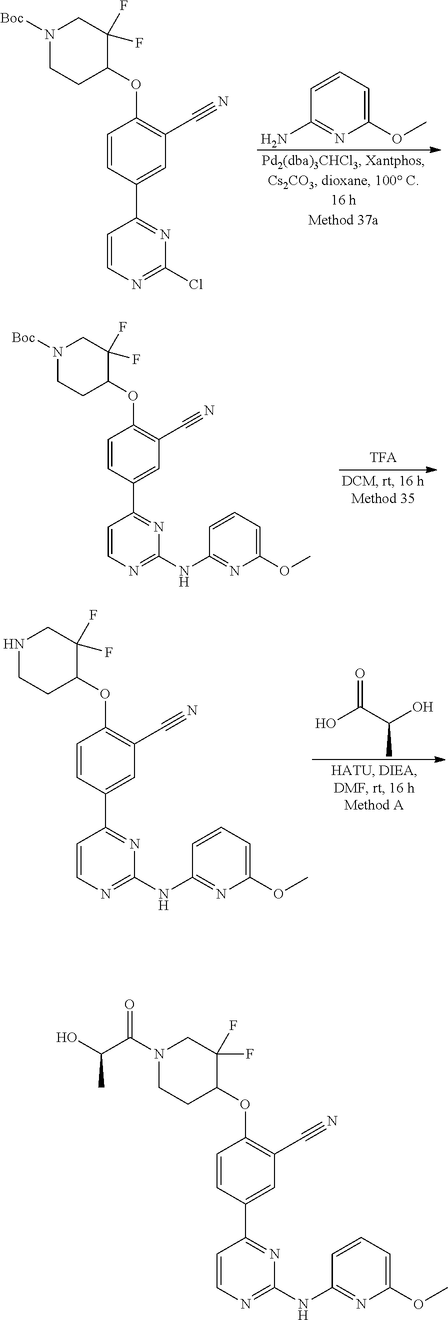

2-[(3,3-difluoropiperidin-4-yl)oxy]-5-[2-([2-methoxy-4-[4-(oxetan-3-yl)piperazin-1-yl]phenyl]amino)pyrimidin-4-yl]benzonitrile: The title compound was prepared from tert-butyl 4-[4-(2-chloropyrimidin-4-yl)-2-cyanophenoxy]-3,3-difluoropiperidine-1-carboxylate and 6-methoxypyridin-2-amine using Method 37a and 35. The final product was purified by prep-HPLC under the following condition: column, Atlantis HILIC OBD C18 Column, 150×19 mm, 5 um; mobile phase, acetonitrile in water (with 10 mmol/L NR₄HCO₃ and 0.1% NH₃.H₂O), 30% to 60% gradient in 8 min; detector, UV 254 nm. 2-[(3,3-difluoropiperidin-4-yl)oxy]-5-[2-([2-methoxy-4-[4-(oxetan-3-yl)piperazin-1-yl]phenyl]amino)pyrimidin-4-yl]benzonitrile was obtained as white solid (6 mg, 1.6% for 2 steps). HPLC: 98.1% purity, RT=4.60 min. MS: m/z=439.3 [M+H]⁺. ¹H NMR (300 MHz, DMSO-d₆, ppm) δ 9.63 (s, 1H), 8.66-8.56 (m, 2H), 8.55-8.45 (m, 1H), 7.90-7.80 (m, 1H), 7.73-7.55 (m, 3H), 6.46-6.36 (m, 1H), 5.29-5.10 (m, 1H), 3.84 (s, 3H), 3.18-3.11 (m, 1H), 3.01-2.76 (m, 2H), 2.73-2.66 (m, 1H), 2.56-2.49 (m, 1H), 2.12-1.70 (m, 2H).

2-([3,3-difluoro-1-[(2S)-2-hydroxypropanoyl]piperidin-4-yl]oxy)-5-[2-[(6-methoxypyridin-2-yl)amino]pyrimidin-4-yl]benzonitrile: The title compound was prepared from 2-[(3,3-difluoropiperidin-4-yl)oxy]-5-[2-[(6-methoxypyridin-2-yl)amino]pyrimidin-4-yl]benzonitrile and (2S)-2-hydroxypropanoic acid using Method A. The final product was purified by prep-HPLC under the following condition: column, XBridge Prep Phenyl OBD C18 Column, 150×30 mm, 5 um; mobile phase, acetonitrile in water (with 10 mmol/L NH₄HCO₃ and 0.1% NH₃.H₂O), 30% to 45% gradient in 8 min; detector, UV 254 nm. 2-([3,3-difluoro-1-[(2S)-2-hydroxypropanoyl] piperidin-4-yl]oxy)-5-[2-[(6-methoxypyridin-2-yl)amino]pyrimidin-4-yl]benzonitrile was obtained as white solid (25 mg, 22%). HPLC: 99.0% purity, RT=5.72 min. MS: m/z=511.4 [M+H]⁺. ¹H NMR (300 MHz, DMSO-d₆, ppm) δ 9.63 (s, 1H), 8.67-8.58 (m, 2H), 8.58-8.48 (m, 1H), 7.90-7.80 (m, 1H), 7.74-7.56 (m, 3H), 6.46-6.37 (m, 1H), 5.41-5.35 (m, 1H), 5.28-5.17 (m, 1H), 4.54-4.43 (m, 1H), 4.25-3.95 (m, 2H), 3.85 (s, 3H), 4.25-3.95 (m, 2H), 2.26-1.73 (m, 2H), 1.21 (d, J=6.4 Hz, 3H).

### Example 329: 2-[[33-difluoro-1-(2-hydroxypropanoyl)piperidin-4-yl]oxy]-5-[2-([6-methoxy-5-[1-(oxetan-3-yl)piperidin-4-yl]pyridin-2-yl]amino)pyrimidin-4-yl]benzonitrile

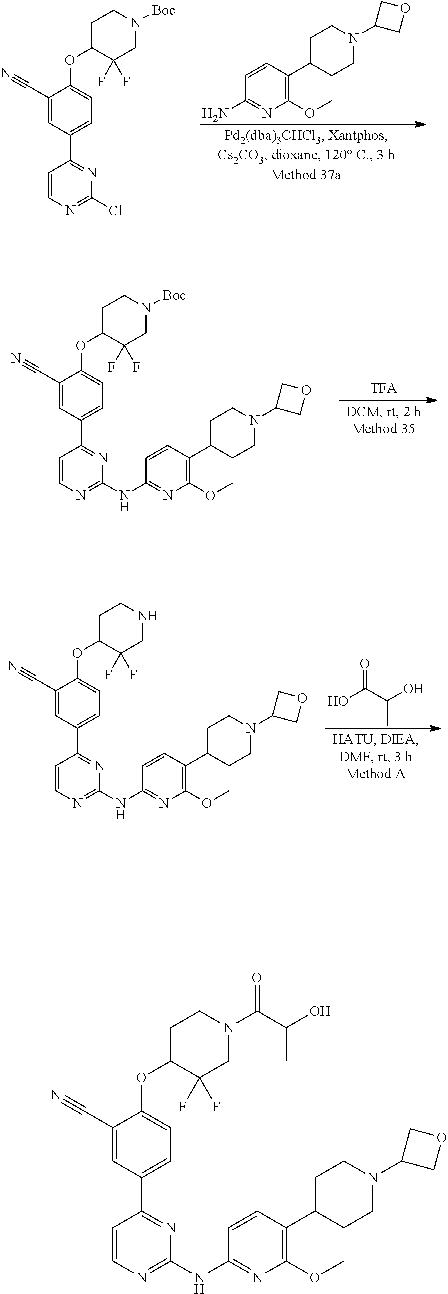

2-[(3,3-difluoropiperidin-4-yl)oxy]-5-[2-([6-methoxy-5-[1-(oxetan-3-yl)piperidin-4-yl]pyridin-2-yl]amino)pyrimidin-4-yl]benzonitrile: The title compound was prepared from tert-butyl 4-[4-(2-chloropyrimidin-4-yl)-2-cyanophenoxy]-3,3-difluoropiperidine-1-carboxylate and 6-methoxy-5-[1-(oxetan-3-yl)piperidin-4-yl]pyridin-2-amine using Method 37a and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 35% to 60% gradient in 8 min; detector, UV 254 nm. 2-[[3,3-difluoro-1-(2-hydroxypropanoyl)piperidin-4-yl]oxy]-5-[2-([6-methoxy-5-[1-(oxetan-3-yl)piperidin-4-yl]pyridin-2-yl]amino)pyrimidin-4-yl]benzonitrile was obtained as white solid (9 mg, 9.4% for 2 steps). HPLC: 94.3% purity, RT=13.87 min. MS: m/z=578.2 [M+H]⁺. ¹H NMR (300 MHz, DMSO-d₆, ppm) δ 9.52 (s, 1H), 8.66-8.57 (m, 2H), 8.56-8.46 (m, 1H), 7.85-7.75 (m, 1H), 7.69-7.54 (m, 3H), 5.26-5.20 (m, 1H), 4.60-4.49 (m, 2H), 4.50-4.40 (m, 2H), 3.89 (s, 3H), 3.52-3.38 (m, 1H), 3.22-3.08 (m, 1H), 3.04-2.60 (m, 7H), 2.08-2.01 (m, 1H), 1.90-1.80 (m, 3H), 1.78-1.55 (m, 4H).

2-[[3,3-difluoro-1-(2-hydroxypropanoyl)piperidin-4-yl]oxy]-5-[2-([6-methoxy-5-[1-(oxetan-3-yl)piperidin-4-yl]pyridin-2-yl]amino)pyrimidin-4-yl]benzonitrile: The title compound was prepared from 2-[(3,3-difluoropiperidin-4-yl)oxy]-5-[2-([6-methoxy-5-[1-(oxetan-3-yl)piperidin-4-yl]pyridin-2-yl]amino)pyrimidin-4-yl]benzonitrile and 2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 35% to 60% gradient in 8 min; detector, UV 254 nm. 2-[[3,3-difluoro-1-(2-hydroxypropanoyl)piperidin-4-yl]oxy]-5-[2-([6-methoxy-5-[1-(oxetan-3-yl)piperidin-4-yl]pyridin-2-yl]amino)pyrimidin-4-yl]benzonitrile was obtained as white solid (25 mg, 39%). HPLC: 95.5% purity, RT=4.66 min. MS: m/z=650.0 [M+H]VH NMR (300 MHz, DMSO-d₆, ppm) δ 9.53 (s, 1H), 8.67-8.58 (m, 2H), 8.59-8.49 (m, 1H), 7.85-7.75 (m, 1H), 7.73-7.63 (m, 1H), 7.63-7.54 (m, 2H), 5.43-5.36 (m, 1H), 5.30-5.19 (m, 1H), 4.60-4.39 (m, 5H), 4.33-3.97 (m, 2H), 3.89 (s, 3H), 3.85-3.54 (m, 2H), 3.45-3.34 (m, 1H), 2.85-2.74 (m, 2H), 2.74-2.63 (m, 1H), 2.24-1.90 (m, 2H), 1.91-1.78 (m, 2H), 1.78-1.55 (m, 4H), 1.27-1.18 (m, 3H).

### Example 330: 2-([3,3-difluoro-1-[(2R)-2-hydroxypropanoyl]piperidin-4-yl]oxy)-5-[2-([6-methoxy-5-[1-(oxetan-3-yl)piperidin-4-yl]pyridin-2-yl]amino)pyrimidin-4-yl]benzonitrile

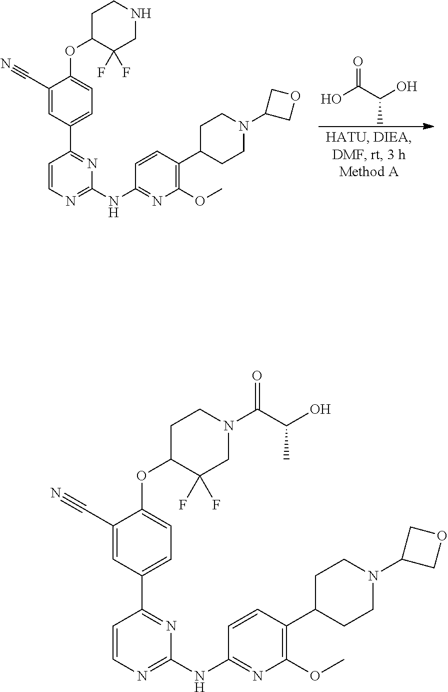

The title compound was prepared from 2-[(3,3-difluoropiperidin-4-yl)oxy]-5-[2-([6-methoxy-5-[1-(oxetan-3-yl)piperidin-4-yl]pyridin-2-yl]amino)pyrimidin-4-yl]benzonitrile and (2R)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NR₄HCO₃ and 0.1% NH₃.H₂O), 32% to 60% gradient in 8 min; detector, UV 254 nm. 2-([3,3-difluoro-1-[(2R)-2-hydroxypropanoyl]piperidin-4-yl]oxy)-5-[2-([6-methoxy-5-[1-(oxetan-3-yl)piperidin-4-yl]pyridin-2-yl]amino)pyrimidin-4-yl]benzonitrile was obtained as white solid (18 mg, 25%). HPLC: 90.0% purity, RT=4.58 min. MS: m/z=650.1 [M+H]⁺. ¹H NMR (300 MHz, DMSO-d₆, ppm) δ 9.50 (s, 1H), 8.65-8.47 (m, 3H), 7.83-7.74 (m, 1H), 7.71-7.61 (m, 1H), 7.61-7.52 (m, 2H), 5.37 (br s, 1H), 5.27-5.17 (m, 1H), 4.58-4.37 (m, 5H), 4.29-3.93 (m, 1H), 3.87 (s, 3H), 3.82-3.54 (m, 2H), 3.45-3.35 (m, 1H), 2.83-2.62 (m, 3H), 2.20-1.89 (m, 2H), 1.90-1.76 (m, 2H), 1.76-1.53 (m, 4H), 1.28-1.14 (m, 3H).

### Example 331: 2-[[(3S,4R)-3-fluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile

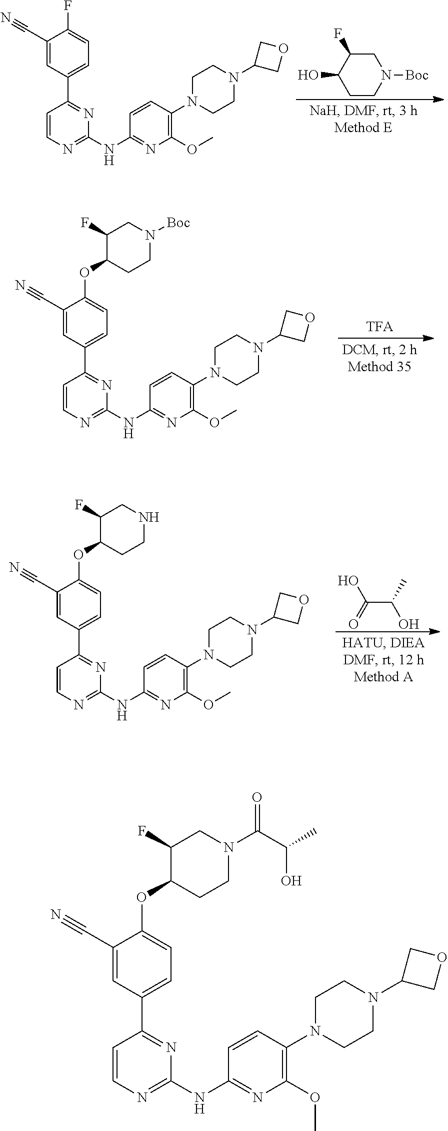

2-[[(3S,4R)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile: The title compound was prepared from tert-butyl (3S,4R)-3-fluoro-4-hydroxypiperidine-1-carboxylate and 2-fluoro-5-[2-([6-methoxy-5-[4-(oxetan-3-yl)piperazin-1-yl]pyridin-2-yl]amino)pyrimidin-4-yl]benzonitrile using Method E and 35. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 23% to 53% gradient in 8 min; detector, UV 254 nm. 2-[[(3S,4R)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile was obtained as a light yellow solid (4.6 mg, 6% for 2 steps). HPLC: 99.8% purity, RT=3.34 min. MS: m/z=561.2 [M+H]⁺. ¹H NMR (400 MHz, DMSO-d₆.ppm) δ9.35 (s, 1H), 8.60-8.54 (m, 2H), 8.50-8.43 (m, 1H), 7.77-7.71 (m, 1H), 7.60-7.54 (m, 1H), 7.54-7.49 (m, 1H), 7.31-7.24 (m, 1H), 5.10-4.97 (m, 1H), 4.92-4.72 (m, 1H), 4.56 (t, J=6.5 Hz, 2H), 4.47 (t, J=6.1 Hz, 2H), 3.90 (s, 3H), 3.52-3.41 (m, 1H), 3.18-3.07 (m, 1H), 3.05-2.93 (m, 4H), 2.94-2.79 (m, 2H), 2.68-2.58 (m, 1H), 2.43-2.39 (m, 4H), 2.15-2.11 (m, 1H), 1.91-1.77 (m, 2H).

2-[[(3S,4R)-3-fluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile: The title compound was prepared from 2-[[(3S,4R)-3-fluoropiperidin-4-yl]oxy]-5-[2-([6-methoxy-5-[4-(oxetan-3-yl)piperazin-1-yl]pyridin-2-yl]amino)pyrimidin-4-yl]benzonitrile and (2S)-2-hydroxypropanoic acid using Method A. The final product was purified by prep-HPLC under the following condition: column, XBridge Prep OBD C18 Column, 150×30 mm, 5 um; mobile phase, acetonitrile in water (with 10 mmol/L NH₄HCO₃ and 0.1% NH₃.H₂O), 23% to 53% gradient in 8 min; detector, UV 254 nm. 2-[[(3S,4R)-3-fluoro-1-[(2S)-2-hydroxypropanoyl]piperidin-4-yl]oxy]-5-[2-([6-methoxy-5-[4-(oxetan-3-yl)piperazin-1-yl]pyridin-2-yl]amino)pyrimidin-4-yl]benzonitrile was obtained as a light yellow solid (209 mg, 32%). HPLC: 99.8% purity, RT=3.98 min. MS: m/z=633.2 [M+H]⁺. ¹H NMR (400 MHz, DMSO-d₆, ppm) δ9.36 (s, 1H), 8.61-8.56 (m, 2H), 8.53-8.46 (m, 1H), 7.77-7.71 (m, 1H), 7.66-7.59 (m, 1H), 7.56-7.50 (m, 1H), 7.30-7.24 (m, 1H), 5.25-4.89 (m, 3H), 4.60-4.43 (m, 5H), 4.40-3.94 (m, 2H), 3.90 (s, 3H), 3.74-3.58 (m, 0.5H), 3.52-3.34 (m, 2H), 3.23-3.13 (m, 0.5H), 3.00-2.96 (m, 4H), 2.43-2.38 (m, 4H), 2.07-1.75 (m, 2H), 1.22 (d, J=6.6 Hz, 3H).

### Example 332: TBK biochemical assay

Test compounds were transferred into Labcyte polypropylene 384 well plates (P055-25) and diluted to 3 mM using DMSO. 3 mM test compounds were dispensed using Labcyte ECHO dose response module into Greiner 784075 plates (columns 3-12 and 13-22, 10 point 1:4) so that high concentration was 30 uM final. 100 uM of a reference compound (1 uM final high concentration). Backfilling was performed if necessary so that all wells contain 1% DMSO final:

add 75 nl DMSO/well into columns 1, 2 and 24 using Labcyte Echo.
add 75 nl 1.0 mM staurosporine/well into column 23 using Labcyte Echo (10 uM final)
add 4.5 ul enzyme/well using multidrop dispenser
add 3 ul substrate/well using multidrop dispenser
incubate at 25° C. in Heidolph incubator for 90 min.
add 7.5 ul 2× stop buffer using multidrop dispenser
read on labchip ez reader II using TBK1.job

Raw data files were opened in the Caliper LabChip Reviewer program (Version 3.0.265.0 SP2) and peak assignments were adjusted to reflect “substrate first” with the software's post-run analysis options. A spline-fit baseline was applied using the software's analysis algorithm.

### IKKε Biochemical Assay

Test compounds were transferred into Labcyte polypropylene 384 well plates (P055-25) and diluted to 3 mM using DMSO. 3 mM test compounds were dispensed using Labcyte ECHO dose response module into Greiner 784075 plates (columns 3-12 and 13-22, 10 point 1:4) so that high concentration was 30 uM final. 100 uM of a reference compound (1 uM final high concentration). Backfilling was performed if necessary so that all wells contain 1% DMSO final:

add 75 nl DMSO/well into columns 1, 2 and 24 using Labcyte Echo.
add 75 nl 1.0 mM staurosporine/well into column 23 using Labcyte Echo (10 uM final)
add 4.5 ul enzyme/well using multidrop dispenser
add 3 ul substrate/well using multidrop dispenser
incubate at 25° C. for 90 min.
add 7.5 ul 2× stop buffer
read on labchip ez reader II using IKKε.

Raw data files were opened in the Caliper LabChip Reviewer program (Version 3.0.265.0 SP2) and peak assignments were adjusted to reflect “substrate first” with the software's post-run analysis options. A spline-fit baseline was applied using the software's analysis algorithm.

The purpose of the pIRF3 immunocytochemistry cell based assay was to identify small molecules which modulates TBK/IKKε kinase activity through on target substrate phosphorylation of the IRF-3 protein. On the first day of the experiment, MDA-MB-468 cells were plated in 384 well, black, clear-bottom, Poly D lysine coated plates at a density of 5000 cells/well in 45 ul of complete DMEM and allowed to adhere overnight. On the second day compounds were added to cells at a starting concentration of 10 uM with a serial dilution of 3-fold for a total of 10 points. The cells were incubated for 1 hr at 37° C. Cells were then stimulated with Poly(I:C) at a final concentration of 10 ug/ml, and were incubated for 2 hr at 37° C. Following the incubation, media was removed from the wells and the cells were fixed with 4% PFA for 15 min at RT. Cells were washed at least 3 times with PBS, and then permeabilized with ice-cold methanol for 10 min at RT. The washing step was repeated and the cells were then blocked using 10% goat serum/1% BSA, made up in PBS and allowed to incubate at RT for 1 hr. The cells were washed again and then treated with an anti-pIRF3 antibody at 4° C. overnight (1:250 dilution of Abeam ab76493 in PBS containing 1% BSA). On the third day the primary antibody was washed off and pIRF3 was detected by adding the secondary antibody conjugated to AlexaFluor488 (1:200 dilution of secondary antibody in PBS containing 1% BSA) for 1 hr at RT. Cells were washed and then counterstained with PI/RNase staining buffer for 15 min at RT and read on the Acumen Explorer laser scanning cytometer. The percentage of phosphorylation of the IRF-3 protein was calculated using the following algorithm, a modified version of the mean half width intensity (pIRF3 staining)/(PI staining or # of cells)×100%). IC50 curves were generated using the Genedata software.

Results are given in the following table.

### D IC₅₀>5 μM

C IC₅₀ ranges from 1 μM-5 μM
B IC₅₀ ranges from 100 nM-1.0 μM

### A IC₅₀<100 nM

| Example | TBK1_IC50 | IKKε_IC50 |
|---|---|---|
| 1 | A | A |
| 2 | A | A |
| 3 | A | A |
| 4 | A | A |
| 5 | A | A |
| 6 | A | A |
| 7 | A | A |
| 8 | A | A |
| 9 | A | A |
| 10 | A | A |
| 11 | A | A |
| 12 | A | A |
| 13 | A | A |
| 14 | A | A |
| 15 | A | A |
| 16 | A | A |
| 17 | A | A |
| 18 | A | A |
| 19 | A | A |
| 20 | A | A |
| 21 | A | A |
| 22 | B | B |
| 23 | A | A |
| 24 | B | B |
| 25 | A | A |
| 26 | A | A |
| 27 | B | B |
| 28 | A | A |
| 29 | A | A |
| 30 | A | A |
| 31 | A | A |
| 32 | A | A |
| 33 | A | A |
| 34 | A | A |
| 35 | A | A |
| 36 | A | A |
| 37 | A | A |
| 38 | A | A |
| 39 | A | A |
| 40 | A | A |
| 41 | A | A |
| 42 | A | A |
| 43 | A | A |
| 44 | C | D |
| 45 | D | C |
| 46 | A | A |
| 47 | A | B |
| 48 | A | A |
| 49 | B | B |
| 50 | A | A |
| 51 | D | D |
| 52 | C | C |
| 53 | B | B |
| 54 | A | A |
| 55 | D | D |
| 56 | B | B |
| 57 | A | A |
| 58 | C | D |
| 59 | B | A |
| 60 | D | D |
| 61 | B | B |
| 62 | A | B |
| 63 | A | A |
| 64 | A | A |
| 65 | A | A |
| 66 | A | A |
| 67 | A | A |
| 68 | A | A |
| 69 | B | B |
| 70 | B | B |
| 71 | B | B |
| 72 | A | A |
| 73 | A | A |
| 74 | A | A |
| 75 | A | A |
| 76 | A | A |
| 77 | A | A |
| 78 | A | A |
| 79 | A | A |
| 81 | C | C |
| 82 | D | C |
| 83 | D | C |
| 84 | B | A |
| 85 | B | B |
| 86 | B | B |
| 87 | A | A |
| 88 | B | B |
| 89 | A | A |
| 90 | A | A |
| 91 | A | A |
| 92 | A | A |
| 93 | A | A |
| 94 | A | A |
| 95 | A | A |
| 96 | A | A |
| 97 | A | A |
| 98 | A | A |
| 99 | A | A |
| 100 | A | A |
| 101 | A | A |
| 102 | A | A |
| 103 | A | A |
| 104 | A | A |
| 105 | A | A |
| 106 | A | A |
| 107 | A | A |
| 108 | A | A |
| 109 | A | A |
| 110 | A | A |
| 111 | A | A |
| 112 | A | A |
| 113 | A | A |
| 114 | A | A |
| 115 | A | A |
| 116 | A | A |
| 117 | A | A |
| 118 | A | A |
| 119 | A | A |
| 120 | A | A |
| 121 | A | A |
| 122 | A | A |
| 123 | A | A |
| 124 | A | A |
| 125 | A | A |
| 126 | A | A |
| 127 | A | A |
| 128 | A | A |
| 129 | A | A |
| 130 | A | A |
| 131 | A | A |
| 132 | A | A |
| 133 | A | A |
| 134 | A | A |
| 135 | A | A |
| 136 | A | A |
| 137 | A | A |
| 138 | A | A |
| 139 | A | A |
| 140 | A | A |
| 141 | A | A |
| 142 | A | A |
| 143 | N/A | N/A |
| 144 | A | A |
| 145 | A | A |
| 146 | A | A |
| 147 | A | A |
| 148 | A | A |
| 149 | A | A |
| 150 | A | A |
| 151 | A | A |
| 152 | A | A |
| 153 | A | A |
| 154 | A | A |
| 155 | A | A |
| 156 | A | A |
| 157 | A | A |
| 158 | A | A |
| 159 | A | A |
| 160 | B | B |
| 161 | A | A |
| 162 | A | A |
| 163 | A | A |
| 164 | A | A |
| 165 | A | A |
| 166 | A | A |
| 167 | A | A |
| 168 | A | A |
| 169 | A | A |
| 170 | A | A |
| 171 | A | A |
| 172 | B | B |
| 173 | B | B |
| 174 | A | A |
| 175 | B | B |
| 176 | B | C |
| 179 | A | A |
| 182 | A | A |
| 183 | A | A |
| 184 | B | B |
| 185 | A | A |
| 186 | A | A |
| 187 | A | A |
| 188 | A | A |
| 189 | A | A |
| 190 | A | A |
| 191 | A | A |
| 192 | A | A |
| 193 | B | B |
| 194 | A | A |
| 195 | B | B |
| 196 | A | A |
| 197 | A | A |
| 198 | A | A |
| 199 | A | A |
| 200 | A | A |
| 201 | A | A |
| 202 | A | A |
| 203 | A | A |
| 204 | A | A |
| 205 | A | A |
| 206 | A | A |
| 207 | A | A |
| 208 | A | A |
| 209 | A | A |
| 210 | A | A |
| 211 | A | A |
| 212 | A | A |
| 213 | A | A |
| 214 | A | A |
| 215 | A | A |
| 216 | A | A |
| 217 | A | A |
| 218 | A | A |
| 219 | A | A |
| 220 | A | A |
| 221 | B | B |
| 222 | A | A |
| 223 | A | A |
| 224 | A | A |
| 225 | A | A |
| 226 | A | A |
| 227 | A | A |
| 228 | A | A |
| 229 | A | A |
| 230 | B | A |
| 231 | A | A |
| 232 | B | B |
| 233 | A | A |
| 234 | A | A |
| 235 | B | B |
| 236 | A | A |
| 237 | A | A |
| 238 | B | B |
| 239 | A | A |
| 240 | B | B |
| 241 | A | A |
| 242 | A | A |
| 243 | A | A |
| 244 | A | A |
| 245 | A | A |
| 246 | A | A |
| 247 | A | A |
| 248 | A | A |
| 249 | A | A |
| 250 | A | A |
| 251 | A | A |
| 252 | A | A |
| 253 | A | A |
| 254 | A | A |
| 255 | A | A |
| 256 | A | A |
| 257 | A | A |
| 258 | A | A |
| 259 | A | A |
| 260 | A | A |
| 261 | A | A |
| 262 | A | A |
| 263 | A | A |
| 264 | A | A |
| 265 | A | A |
| 266 | A | A |
| 267 | A | A |
| 268 | A | B |
| 269 | A | A |
| 270 | A | A |
| 271 | A | A |
| 272 | A | A |
| 273 | A | A |
| 274 | A | A |
| 275 | A | A |
| 276 | A | A |
| 277 | A | A |
| 278 | B | B |
| 279 | A | B |
| 280 | B | B |
| 281 | A | B |
| 282 | A | B |
| 283 | A | A |
| 284 | A | A |
| 285 | B | B |
| 286 | A | B |
| 287 | A | A |
| 288 | A | A |
| 289 | A | A |
| 290 | A | A |
| 291 | A | A |
| 292 | A | A |
| 293 | A | A |
| 294 | A | A |
| 295 | A | A |
| 296 | A | A |
| 297 | A | A |
| 298 | A | A |
| 299 | A | A |
| 300 | A | A |
| 301 | A | A |
| 303 | B | C |
| 304 | A | A |
| 305 | A | A |
| 306 | A | A |
| 307 | A | A |
| 308 | A | A |
| 309 | B | B |
| 310 | A | A |
| 311 | A | A |
| 312 | B | B |
| 313 | A | B |
| 314 | A | A |
| 315 | A | A |
| 316 | A | A |
| 318 | B | B |
| 319 | C | B |
| 32 | A | A |
| 321 | A | A |
| 322 | A | A |
| 323 | A | A |
| 324 | A | A |
| 325 | A | A |
| 326 | A | A |
| 327 | A | A |
| 328 | A | A |
| 329 | A | A |
| 330 | A | A |
| 331 | A | B |

### Example 333. Pharmaceutical Preparations

(A) Injection vials: A solution of 100 g of an active ingredient according to the invention and 5 g of disodium hydrogen phosphate in 31 of bidistilled water is adjusted to pH 6.5 using 2 N hydrochloric acid, sterile filtered, transferred into injection vials, is lyophilized under sterile conditions and is sealed under sterile conditions. Each injection vial contains 5 mg of active ingredient.

(B) Suppositories: A mixture of 20 g of an active ingredient according to the invention is melted with 100 g of soy lecithin and 1400 g of cocoa butter, is poured into moulds and is allowed to cool. Each suppository contains 20 mg of active ingredient.

(C) Solution: A solution is prepared from 1 g of an active ingredient according to the invention, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 11 and sterilized by irradiation. This solution could be used in the form of eye drops.

(D) Ointment: 500 mg of an active ingredient according to the invention is mixed with 99.5 g of Vaseline under aseptic conditions.

(E) Tablets: A mixture of 1 kg of an active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

(F) Coated tablets: Tablets are pressed analogously to Example E and subsequently are coated in a conventional manner with a coating of sucrose, potato starch, talc, tragacanth and dye.

(G) Capsules: 2 kg of an active ingredient according to the invention are introduced into hard gelatin capsules in a conventional manner in such a way that each capsule contains 20 mg of the active ingredient.

(H) Ampoules: A solution of 1 kg of an active ingredient according to the invention in 601 of bidistilled water is sterile filtered, transferred into ampoules, is lyophilized under sterile conditions and is sealed under sterile conditions. Each ampoule contains 10 mg of active ingredient.

(I) Inhalation spray: 14 g of an active ingredient according to the invention are dissolved in 10 l of isotonic NaCl solution, and the solution is transferred into commercially available spray containers with a pump mechanism. The solution could be sprayed into the mouth or nose. One spray shot (about 0.1 ml) corresponds to a dose of about 0.14 mg.

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I, 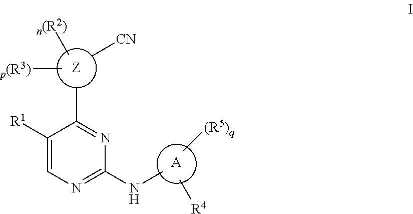
or a pharmaceutically acceptable salt thereof,
wherein:
R¹ is hydrogen, optionally substituted C₁₋₆ aliphatic, —OR, or halogen;
ring Z is phenyl or a 5-6-membered heteroaryl having 1,2, or 3 nitrogens;
each R² is independently —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
each R³ is independently —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
ring A is phenyl or a 5-6-membered heteroaryl having 1,2, or 3 nitrogens;
R⁴ is —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —R, halogen, —OR, —SR, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, a 5-6 membered monocyclic heteroaryl ring having 1-4 heteroatoms independently selected from nitrogen, oxygen, or sulfur, or a 6-12 membered spiro, fused, or bridged bicyclic carbocyclic or heterocyclic ring having 1-4 heteroatoms independently selected from nitrogen, oxygen, or sulfur, each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur, each of which is optionally substituted;
n is 1 or 2;
p is 0,1, or 2; and
q is 0,1, or 2.

2. The compound of claim 1, wherein R¹ is H or F.

3. The compound of claim 1, wherein ring Z is phenyl, pyridine, or pyrimidine.

4. The compound of claim 1, wherein ring Z is

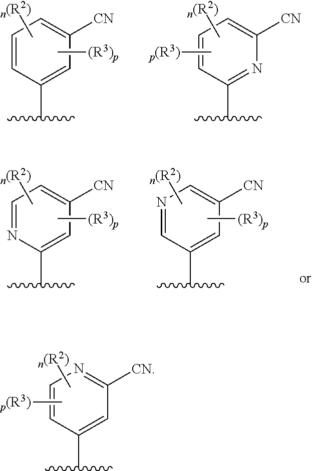

5. The compound of claim 1, wherein each R² is independently —R, halogen, —OR, or —N(R)₂.

6. The compound of claim 1, wherein each R² is independently

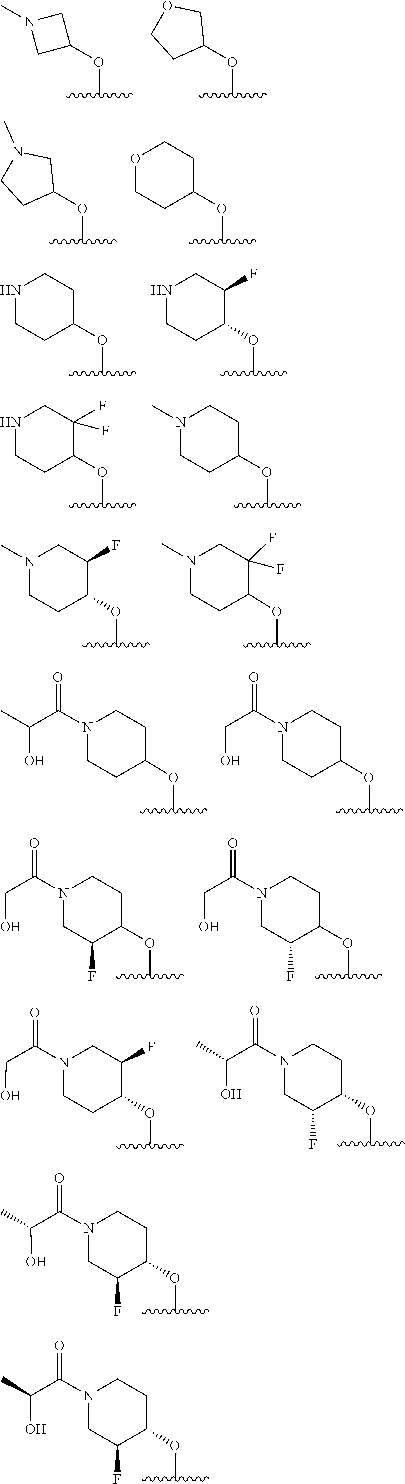

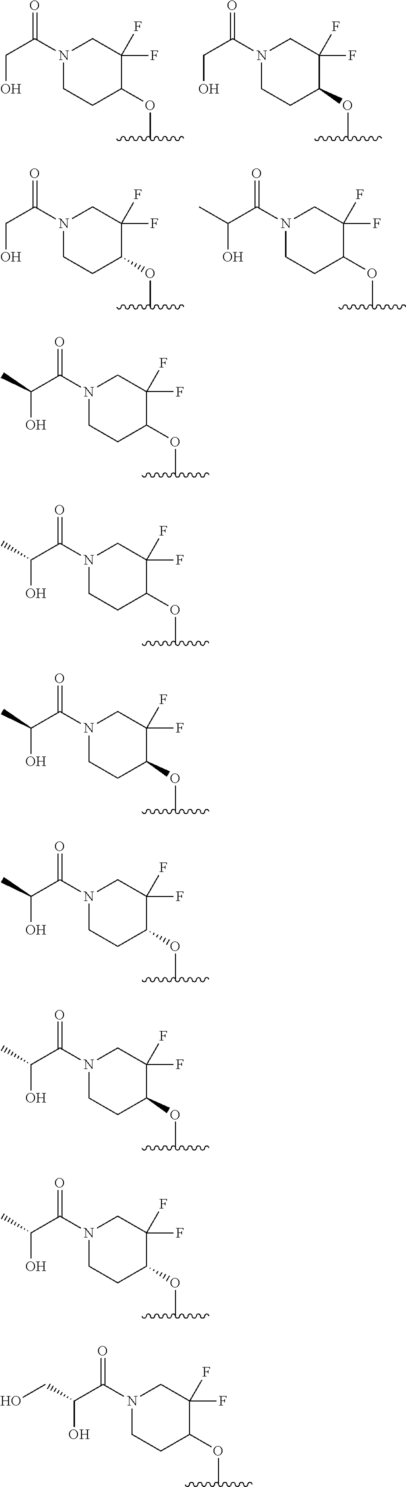

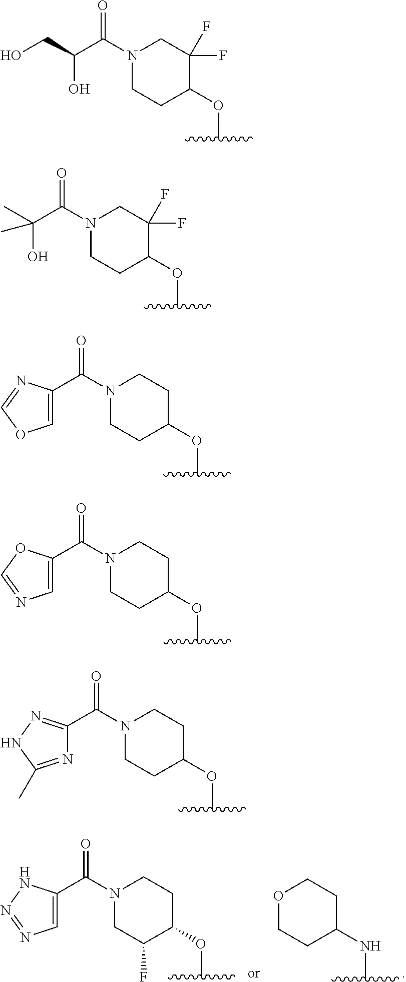

7. The compound of claim 1, wherein each R³ is independently —R, halogen, —OR, or —N(R)₂.

8. The compound of claim 1, wherein ring A is phenyl or pyridyl.

9. The compound of claim 1, wherein ring A is

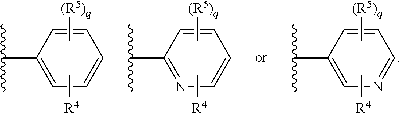

10. The compound of claim 1, wherein R⁴ is —R or —OR.

11. The compound of claim 1, wherein each R⁵ is independently —R, —C(O)R, —CO₂R, —C(O)N(R)₂, —NRC(O)R, or —N(R)₂.

12. The compound of claim 1, wherein each R⁵ is independently

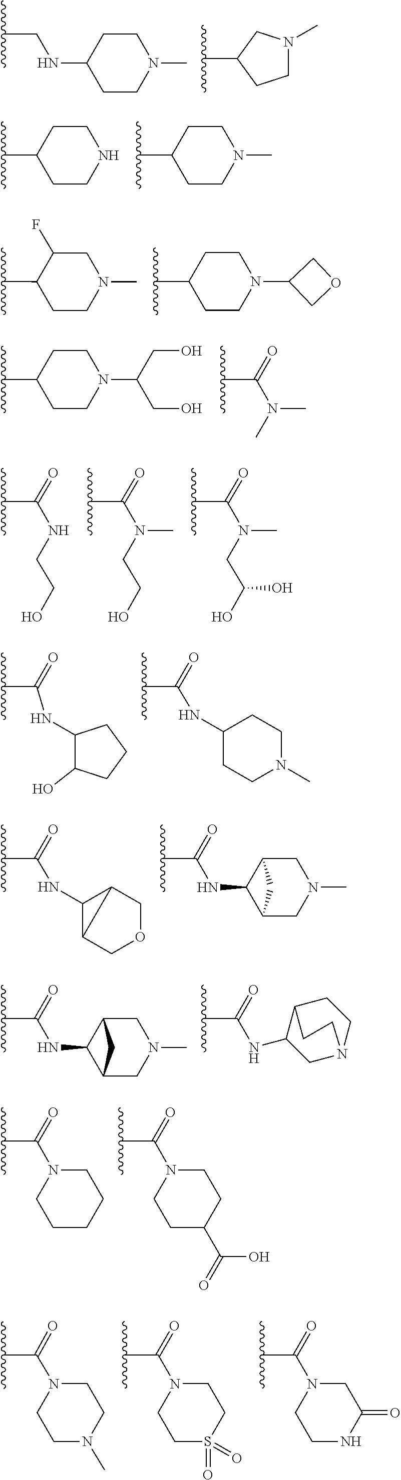

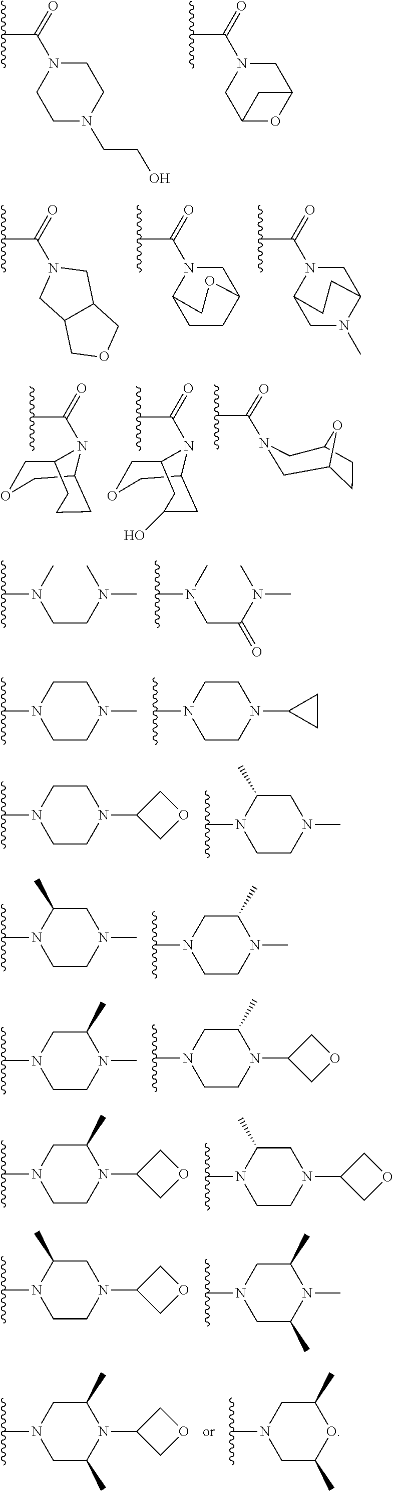

13. The compound of claim 1, of formula II, 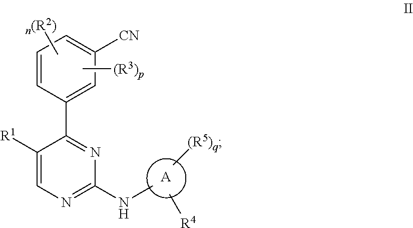
or a pharmaceutically acceptable salt thereof.

14. The compound of claim 1, of formula VI, 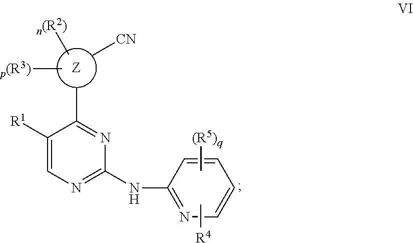
or a pharmaceutically acceptable salt thereof.

15. The compound of claim 1, selected from the group consisting of: 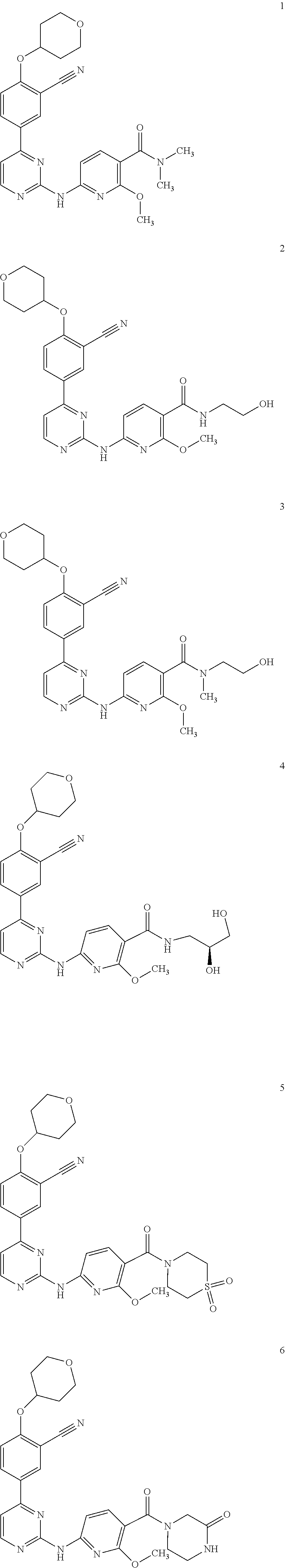 [SMILES: COc1nc(Nc2nccc(-c3ccc(OC4CCOCC4)c(C#N)c3)n2)ccc1C(=O)N(C)C ; COc1nc(Nc2nccc(-c3ccc(OC4CCOCC4)c(C#N)c3)n2)ccc1C(=O)NCCO ; COc1nc(Nc2nccc(-c3ccc(OC4CCOCC4)c(C#N)c3)n2)ccc1C(=O)N(C)CCO ; COc1nc(Nc2nccc(-c3ccc(OC4CCOCC4)c(C#N)c3)n2)ccc1C(=O)NC[C@H](O)CO ; COc1nc(Nc2nccc(-c3ccc(OC4CCOCC4)c(C#N)c3)n2)ccc1C(=O)N1CCS(=O)(=O)CC1 ; COc1nc(Nc2nccc(-c3ccc(OC4CCOCC4)c(C#N)c3)n2)ccc1C(=O)N1CCNC(=O)C1] 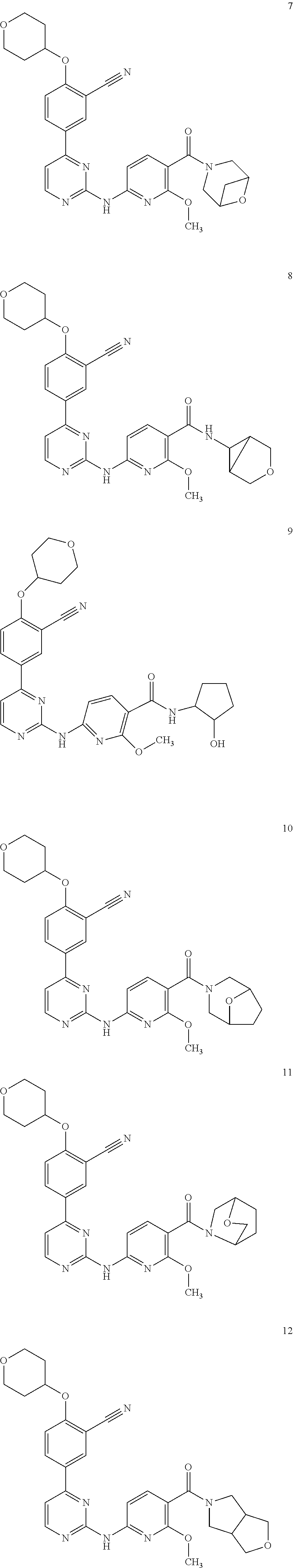 [SMILES: COc1nc(Nc2nccc(-c3ccc(OC4CCOCC4)c(C#N)c3)n2)ccc1C(=O)N1CC2CC(C1)O2 ; COc1nc(Nc2nccc(-c3ccc(OC4CCOCC4)c(C#N)c3)n2)ccc1C(=O)NC1C2COCC21 ; COc1nc(Nc2nccc(-c3ccc(OC4CCOCC4)c(C#N)c3)n2)ccc1C(=O)NC1CCCC1O ; COc1nc(Nc2nccc(-c3ccc(OC4CCOCC4)c(C#N)c3)n2)ccc1C(=O)N1CC2CCC(C1)O2 ; COc1nc(Nc2nccc(-c3ccc(OC4CCOCC4)c(C#N)c3)n2)ccc1C(=O)N1CC2CCC1CO2 ; COc1nc(Nc2nccc(-c3ccc(OC4CCOCC4)c(C#N)c3)n2)ccc1C(=O)N1CC2COCC2C1] 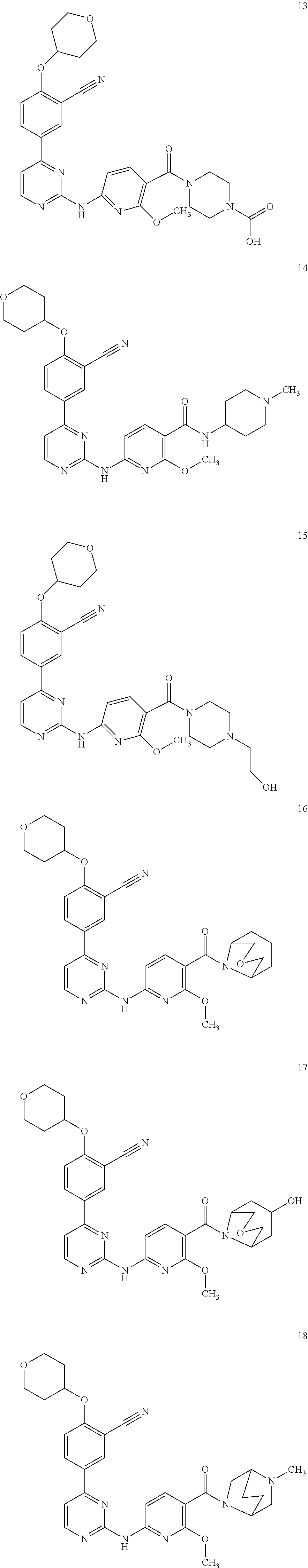 [SMILES: COc1nc(Nc2nccc(-c3ccc(OC4CCOCC4)c(C#N)c3)n2)ccc1C(=O)N1CCN(C(=O)O)CC1 ; COc1nc(Nc2nccc(-c3ccc(OC4CCOCC4)c(C#N)c3)n2)ccc1C(=O)NC1CCN(C)CC1 ; COc1nc(Nc2nccc(-c3ccc(OC4CCOCC4)c(C#N)c3)n2)ccc1C(=O)N1CCN(CCO)CC1 ; COc1nc(Nc2nccc(-c3ccc(OC4CCOCC4)c(C#N)c3)n2)ccc1C(=O)N1C2CCCC1COC2 ; COc1nc(Nc2nccc(-c3ccc(OC4CCOCC4)c(C#N)c3)n2)ccc1C(=O)N1C2COCC1CC(O)C2 ; COc1nc(Nc2nccc(-c3ccc(OC4CCOCC4)c(C#N)c3)n2)ccc1C(=O)N1CC2CCC1CN2C] 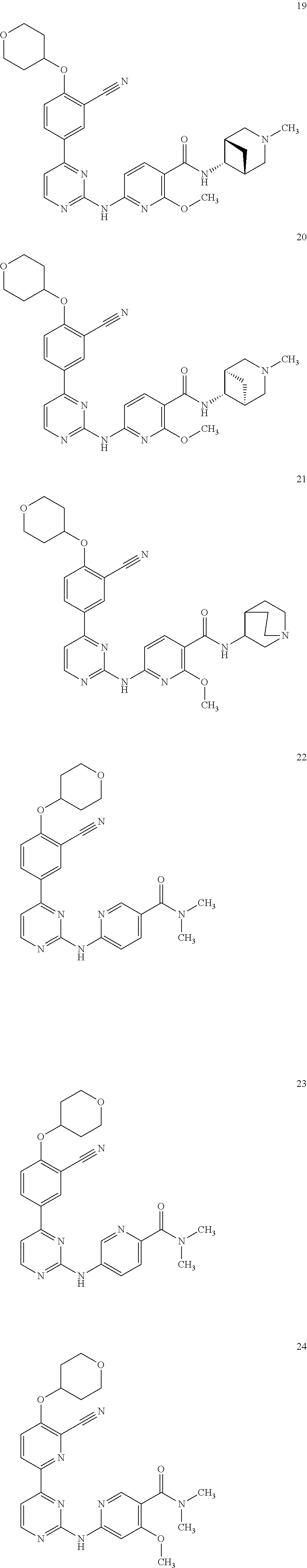 [SMILES: COc1nc(Nc2nccc(-c3ccc(OC4CCOCC4)c(C#N)c3)n2)ccc1C(=O)N[C@H]1[C@@H]2C[C@H]1CN(C)C2 ; COc1nc(Nc2nccc(-c3ccc(OC4CCOCC4)c(C#N)c3)n2)ccc1C(=O)N[C@@H]1[C@@H]2C[C@H]1CN(C)C2 ; COc1nc(Nc2nccc(-c3ccc(OC4CCOCC4)c(C#N)c3)n2)ccc1C(=O)NC1CN2CCC1CC2 ; CN(C)C(=O)c1ccc(Nc2nccc(-c3ccc(OC4CCOCC4)c(C#N)c3)n2)nc1 ; CN(C)C(=O)c1ccc(Nc2nccc(-c3ccc(OC4CCOCC4)c(C#N)c3)n2)cn1 ; COc1cc(Nc2nccc(-c3ccc(OC4CCOCC4)c(C#N)n3)n2)ncc1C(=O)N(C)C] 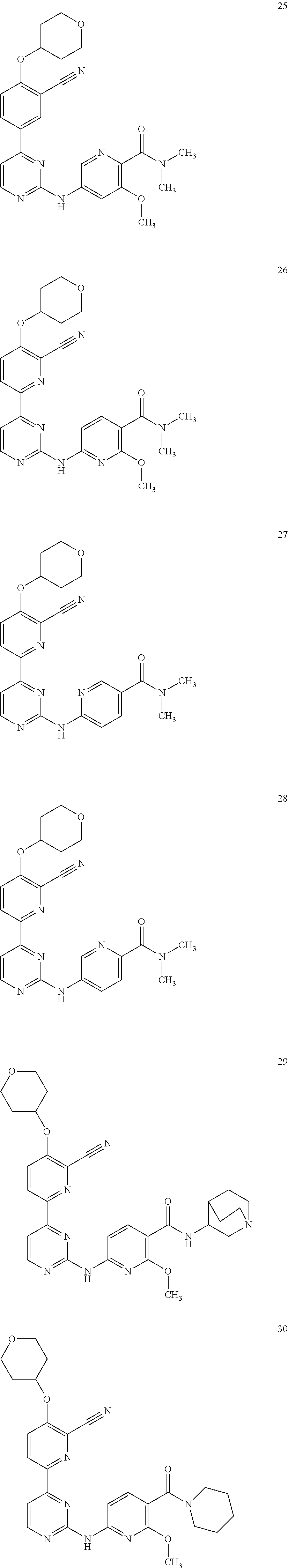 [SMILES: COc1cc(Nc2nccc(-c3ccc(OC4CCOCC4)c(C#N)c3)n2)cnc1C(=O)N(C)C ; COc1nc(Nc2nccc(-c3ccc(OC4CCOCC4)c(C#N)n3)n2)ccc1C(=O)N(C)C ; CN(C)C(=O)c1ccc(Nc2nccc(-c3ccc(OC4CCOCC4)c(C#N)n3)n2)nc1 ; CN(C)C(=O)c1ccc(Nc2nccc(-c3ccc(OC4CCOCC4)c(C#N)n3)n2)cn1 ; COc1nc(Nc2nccc(-c3ccc(OC4CCOCC4)c(C#N)n3)n2)ccc1C(=O)NC1CN2CCC1CC2 ; COc1nc(Nc2nccc(-c3ccc(OC4CCOCC4)c(C#N)n3)n2)ccc1C(=O)N1CCCCC1] 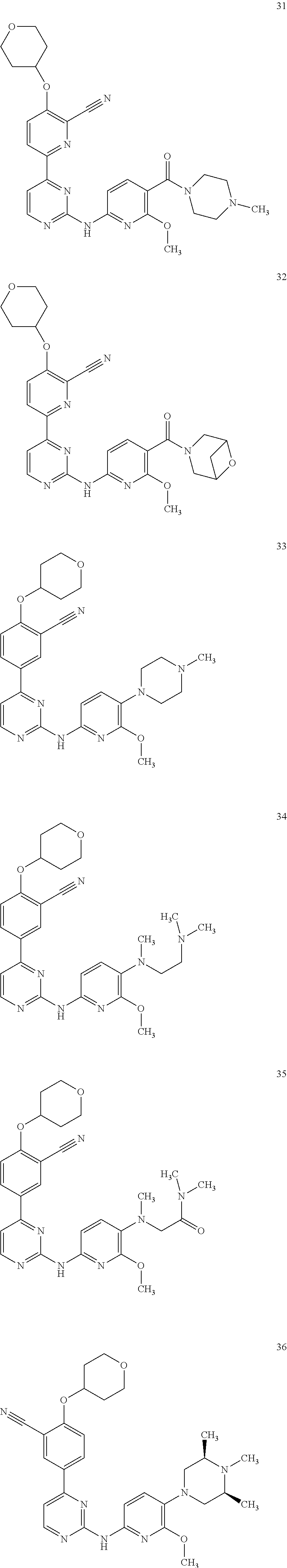 [SMILES: COc1nc(Nc2nccc(-c3ccc(OC4CCOCC4)c(C#N)n3)n2)ccc1C(=O)N1CCN(C)CC1 ; COc1nc(Nc2nccc(-c3ccc(OC4CCOCC4)c(C#N)n3)n2)ccc1C(=O)N1CC2CC(C1)O2 ; COc1nc(Nc2nccc(-c3ccc(OC4CCOCC4)c(C#N)c3)n2)ccc1N1CCN(C)CC1 ; COc1nc(Nc2nccc(-c3ccc(OC4CCOCC4)c(C#N)c3)n2)ccc1N(C)CCN(C)C ; COc1nc(Nc2nccc(-c3ccc(OC4CCOCC4)c(C#N)c3)n2)ccc1N(C)CC(=O)N(C)C ; COc1nc(Nc2nccc(-c3ccc(OC4CCOCC4)c(C#N)c3)n2)ccc1N1C[C@@H](C)N(C)[C@@H](C)C1] 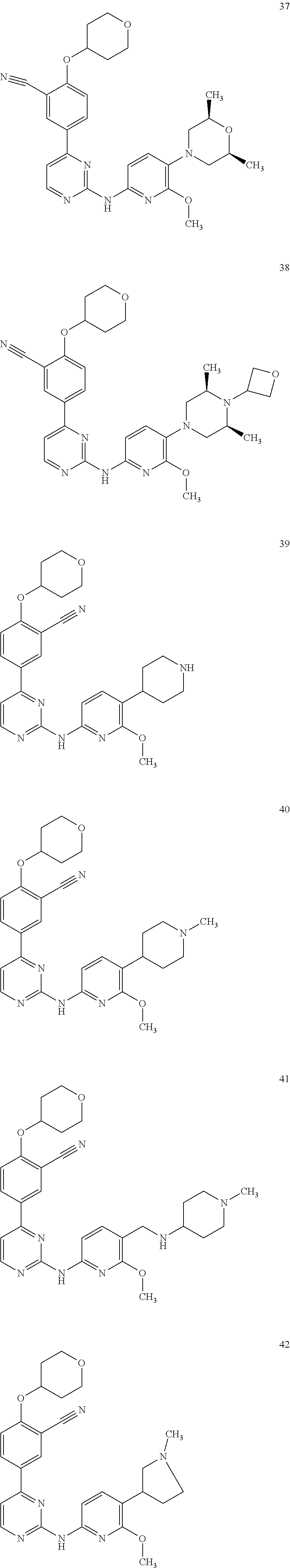 [SMILES: COc1nc(Nc2nccc(-c3ccc(OC4CCOCC4)c(C#N)c3)n2)ccc1N1C[C@@H](C)O[C@@H](C)C1 ; COc1nc(Nc2nccc(-c3ccc(OC4CCOCC4)c(C#N)c3)n2)ccc1N1C[C@@H](C)N(C2COC2)[C@@H](C)C1 ; COc1nc(Nc2nccc(-c3ccc(OC4CCOCC4)c(C#N)c3)n2)ccc1C1CCNCC1 ; COc1nc(Nc2nccc(-c3ccc(OC4CCOCC4)c(C#N)c3)n2)ccc1C1CCN(C)CC1 ; COc1nc(Nc2nccc(-c3ccc(OC4CCOCC4)c(C#N)c3)n2)ccc1CNC1CCN(C)CC1 ; COc1nc(Nc2nccc(-c3ccc(OC4CCOCC4)c(C#N)c3)n2)ccc1C1CCN(C)C1] 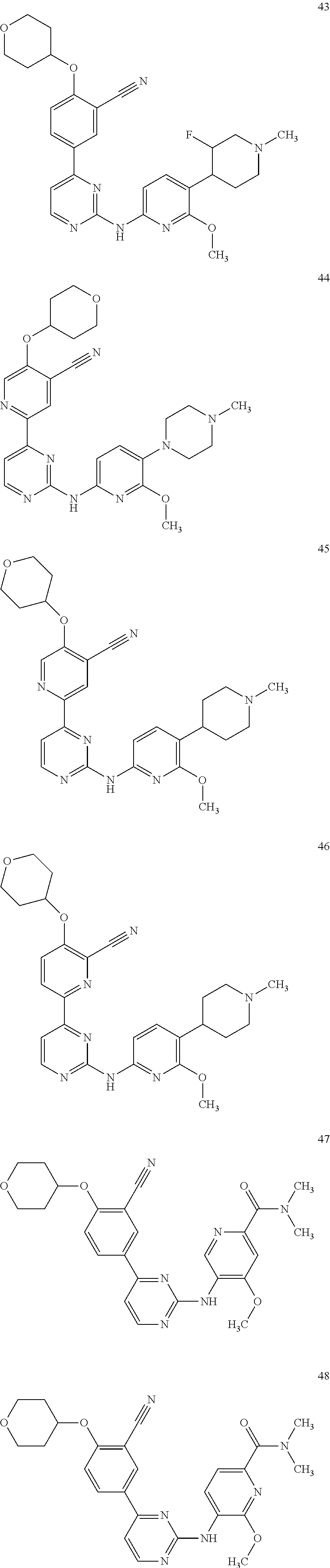 [SMILES: COc1nc(Nc2nccc(-c3ccc(OC4CCOCC4)c(C#N)c3)n2)ccc1C1CCN(C)CC1F ; COc1nc(Nc2nccc(-c3cc(C#N)c(OC4CCOCC4)cn3)n2)ccc1N1CCN(C)CC1 ; COc1nc(Nc2nccc(-c3cc(C#N)c(OC4CCOCC4)cn3)n2)ccc1C1CCN(C)CC1 ; COc1nc(Nc2nccc(-c3ccc(OC4CCOCC4)c(C#N)n3)n2)ccc1C1CCN(C)CC1 ; COc1cc(C(=O)N(C)C)ncc1Nc1nccc(-c2ccc(OC3CCOCC3)c(C#N)c2)n1 ; COc1nc(C(=O)N(C)C)ccc1Nc1nccc(-c2ccc(OC3CCOCC3)c(C#N)c2)n1] 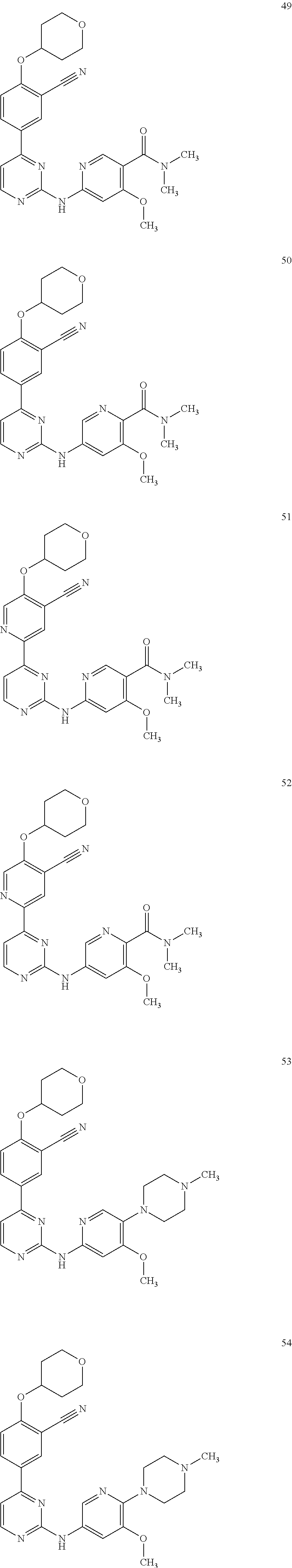 [SMILES: COc1cc(Nc2nccc(-c3ccc(OC4CCOCC4)c(C#N)c3)n2)ncc1C(=O)N(C)C ; COc1cc(Nc2nccc(-c3ccc(OC4CCOCC4)c(C#N)c3)n2)cnc1C(=O)N(C)C ; COc1cc(Nc2nccc(-c3cc(C#N)c(OC4CCOCC4)cn3)n2)ncc1C(=O)N(C)C ; COc1cc(Nc2nccc(-c3cc(C#N)c(OC4CCOCC4)cn3)n2)cnc1C(=O)N(C)C ; COc1cc(Nc2nccc(-c3ccc(OC4CCOCC4)c(C#N)c3)n2)ncc1N1CCN(C)CC1 ; COc1cc(Nc2nccc(-c3ccc(OC4CCOCC4)c(C#N)c3)n2)cnc1N1CCN(C)CC1] 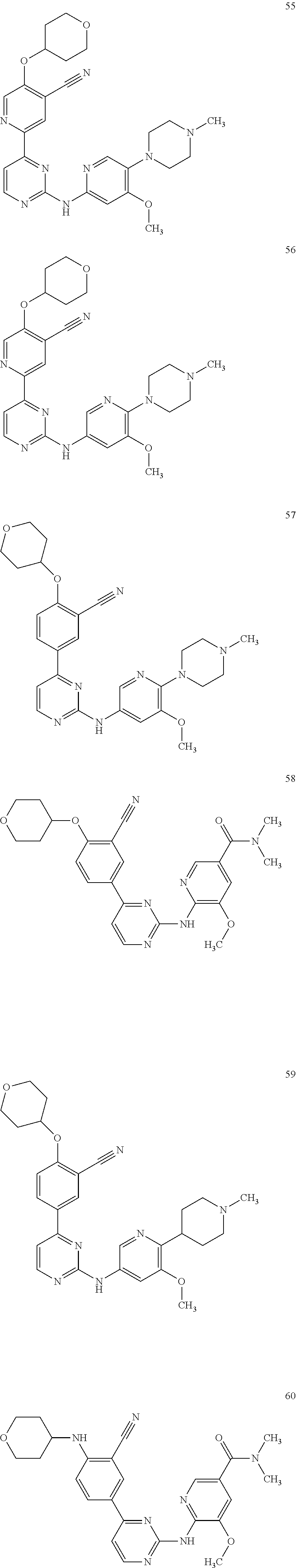 [SMILES: COc1cc(Nc2nccc(-c3cc(C#N)c(OC4CCOCC4)cn3)n2)ncc1N1CCN(C)CC1 ; COc1cc(Nc2nccc(-c3cc(C#N)c(OC4CCOCC4)cn3)n2)cnc1N1CCN(C)CC1 ; COc1cc(Nc2nccc(-c3ccc(OC4CCOCC4)c(C#N)c3)n2)cnc1N1CCN(C)CC1 ; COc1cc(C(=O)N(C)C)cnc1Nc1nccc(-c2ccc(OC3CCOCC3)c(C#N)c2)n1 ; COc1cc(Nc2nccc(-c3ccc(OC4CCOCC4)c(C#N)c3)n2)cnc1C1CCN(C)CC1 ; COc1cc(C(=O)N(C)C)cnc1Nc1nccc(-c2ccc(NC3CCOCC3)c(C#N)c2)n1] 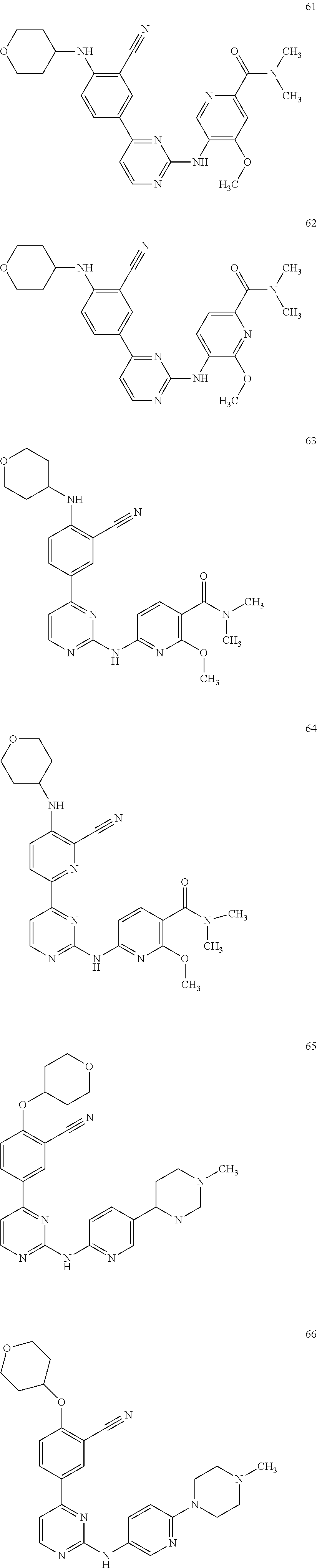 [SMILES: COc1cc(C(=O)N(C)C)ncc1Nc1nccc(-c2ccc(NC3CCOCC3)c(C#N)c2)n1 ; COc1nc(C(=O)N(C)C)ccc1Nc1nccc(-c2ccc(NC3CCOCC3)c(C#N)c2)n1 ; COc1nc(Nc2nccc(-c3ccc(NC4CCOCC4)c(C#N)c3)n2)ccc1C(=O)N(C)C ; COc1nc(Nc2nccc(-c3ccc(NC4CCOCC4)c(C#N)n3)n2)ccc1C(=O)N(C)C ; CN1CCC(c2ccc(Nc3nccc(-c4ccc(OC5CCOCC5)c(C#N)c4)n3)nc2)NC1 ; CN1CCN(c2ccc(Nc3nccc(-c4ccc(OC5CCOCC5)c(C#N)c4)n3)cn2)CC1] 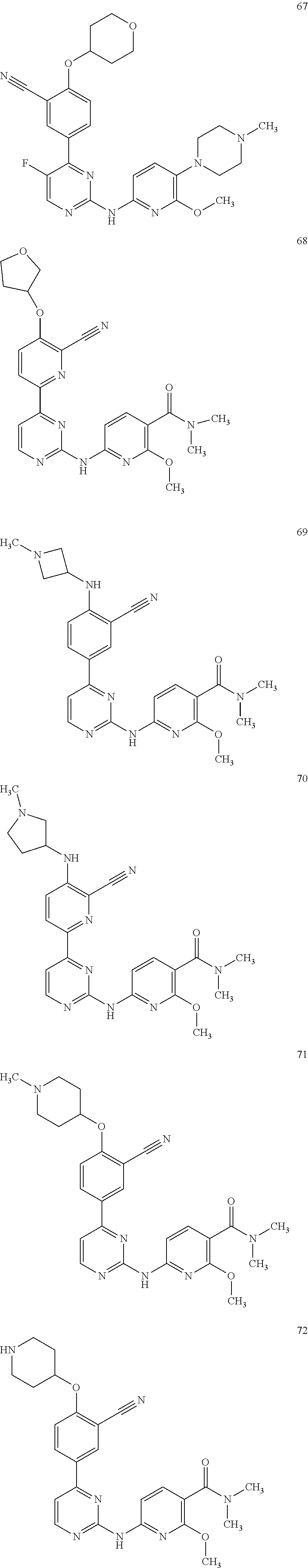 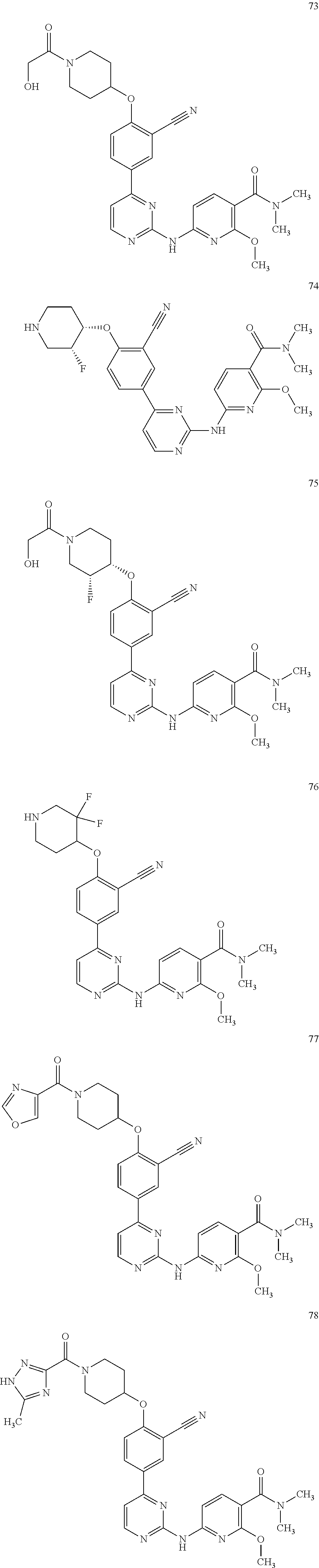 [SMILES: COc1nc(Nc2nccc(-c3ccc(OC4CCN(C(=O)CO)CC4)c(C#N)c3)n2)ccc1C(=O)N(C)C ; COc1nc(Nc2nccc(-c3ccc(O[C@H]4CCNC[C@H]4F)c(C#N)c3)n2)ccc1C(=O)N(C)C ; COc1nc(Nc2nccc(-c3ccc(O[C@H]4CCN(C(=O)CO)C[C@H]4F)c(C#N)c3)n2)ccc1C(=O)N(C)C ; COc1nc(Nc2nccc(-c3ccc(OC4CCNCC4(F)F)c(C#N)c3)n2)ccc1C(=O)N(C)C ; COc1nc(Nc2nccc(-c3ccc(OC4CCN(C(=O)c5cocn5)CC4)c(C#N)c3)n2)ccc1C(=O)N(C)C ; COc1nc(Nc2nccc(-c3ccc(OC4CCN(C(=O)c5n[nH]c(C)n5)CC4)c(C#N)c3)n2)ccc1C(=O)N(C)C] 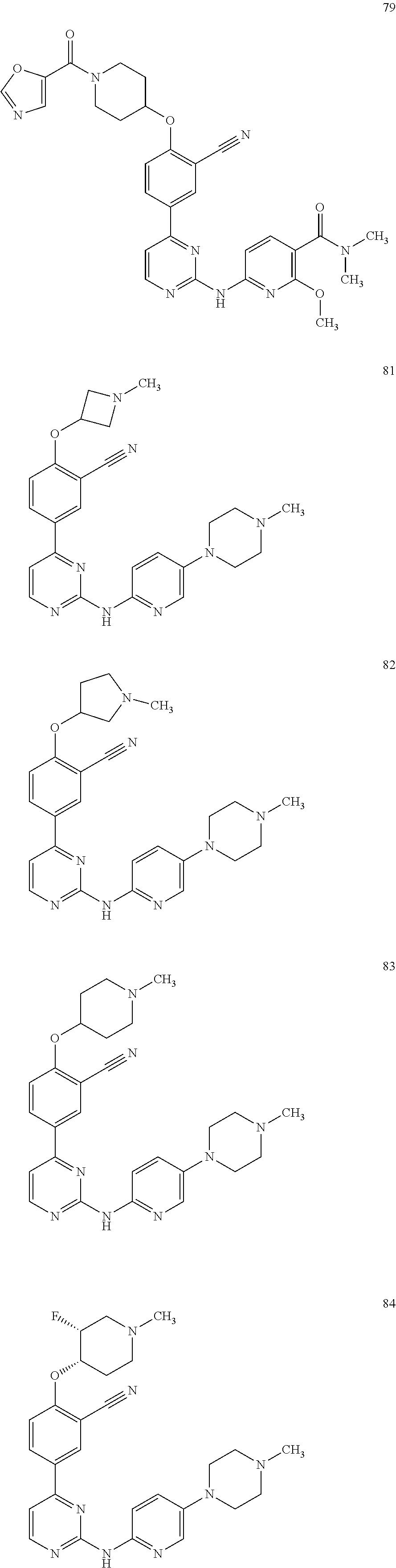 [SMILES: COc1nc(Nc2nccc(-c3ccc(OC4CCN(C(=O)c5cnco5)CC4)c(C#N)c3)n2)ccc1C(=O)N(C)C ; CN1CCN(c2ccc(Nc3nccc(-c4ccc(OC5CN(C)C5)c(C#N)c4)n3)nc2)CC1 ; CN1CCN(c2ccc(Nc3nccc(-c4ccc(OC5CCN(C)C5)c(C#N)c4)n3)nc2)CC1 ; CN1CCC(Oc2ccc(-c3ccnc(Nc4ccc(N5CCN(C)CC5)cn4)n3)cc2C#N)CC1 ; CN1CCN(c2ccc(Nc3nccc(-c4ccc(O[C@H]5CCN(C)C[C@H]5F)c(C#N)c4)n3)nc2)CC1] 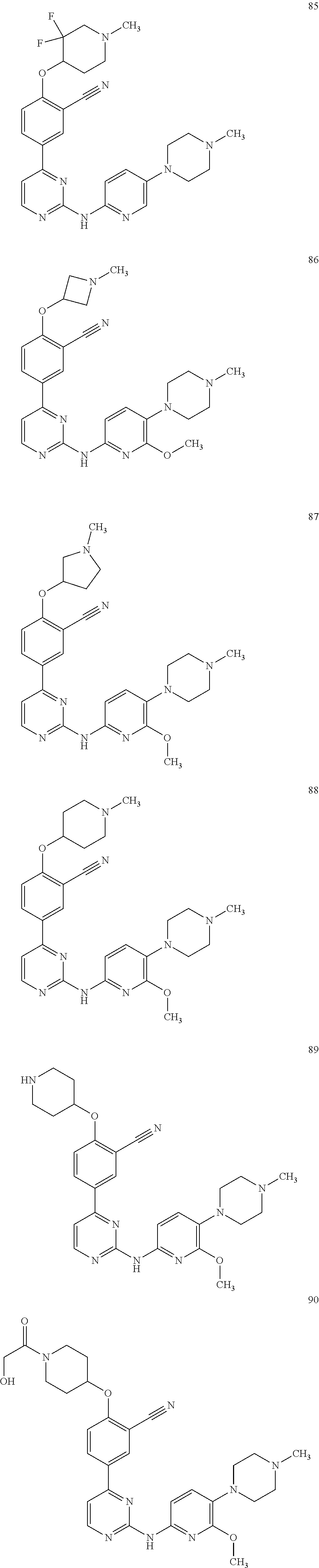 [SMILES: CN1CCN(c2ccc(Nc3nccc(-c4ccc(OC5CCN(C)CC5(F)F)c(C#N)c4)n3)nc2)CC1 ; COc1nc(Nc2nccc(-c3ccc(OC4CN(C)C4)c(C#N)c3)n2)ccc1N1CCN(C)CC1 ; COc1nc(Nc2nccc(-c3ccc(OC4CCN(C)C4)c(C#N)c3)n2)ccc1N1CCN(C)CC1 ; COc1nc(Nc2nccc(-c3ccc(OC4CCN(C)CC4)c(C#N)c3)n2)ccc1N1CCN(C)CC1 ; COc1nc(Nc2nccc(-c3ccc(OC4CCNCC4)c(C#N)c3)n2)ccc1N1CCN(C)CC1 ; COc1nc(Nc2nccc(-c3ccc(OC4CCN(C(=O)CO)CC4)c(C#N)c3)n2)ccc1N1CCN(C)CC1] 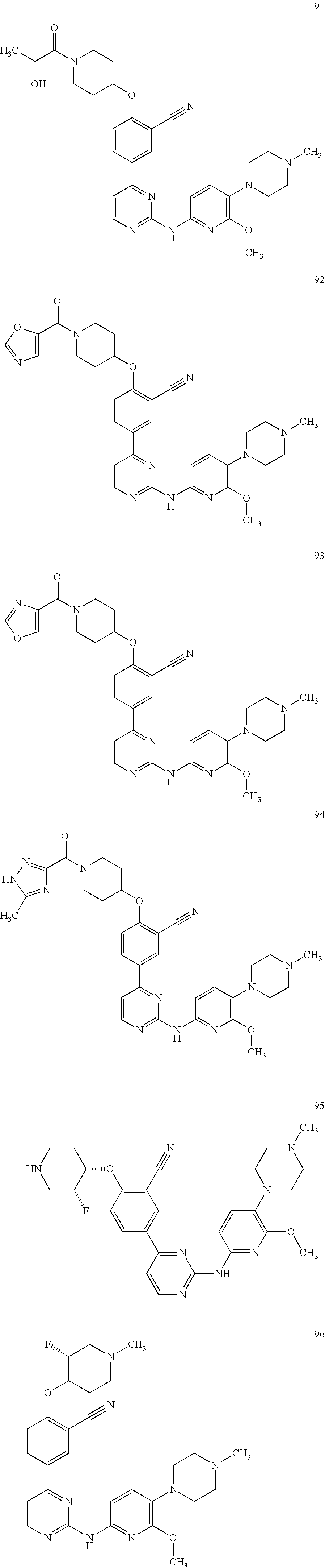 [SMILES: COc1nc(Nc2nccc(-c3ccc(OC4CCN(C(=O)C(C)O)CC4)c(C#N)c3)n2)ccc1N1CCN(C)CC1 ; COc1nc(Nc2nccc(-c3ccc(OC4CCN(C(=O)c5cnco5)CC4)c(C#N)c3)n2)ccc1N1CCN(C)CC1 ; COc1nc(Nc2nccc(-c3ccc(OC4CCN(C(=O)c5cocn5)CC4)c(C#N)c3)n2)ccc1N1CCN(C)CC1 ; COc1nc(Nc2nccc(-c3ccc(OC4CCN(C(=O)c5n[nH]c(C)n5)CC4)c(C#N)c3)n2)ccc1N1CCN(C)CC1 ; COc1nc(Nc2nccc(-c3ccc(O[C@H]4CCNC[C@H]4F)c(C#N)c3)n2)ccc1N1CCN(C)CC1 ; COc1nc(Nc2nccc(-c3ccc(OC4CCN(C)C[C@H]4F)c(C#N)c3)n2)ccc1N1CCN(C)CC1] 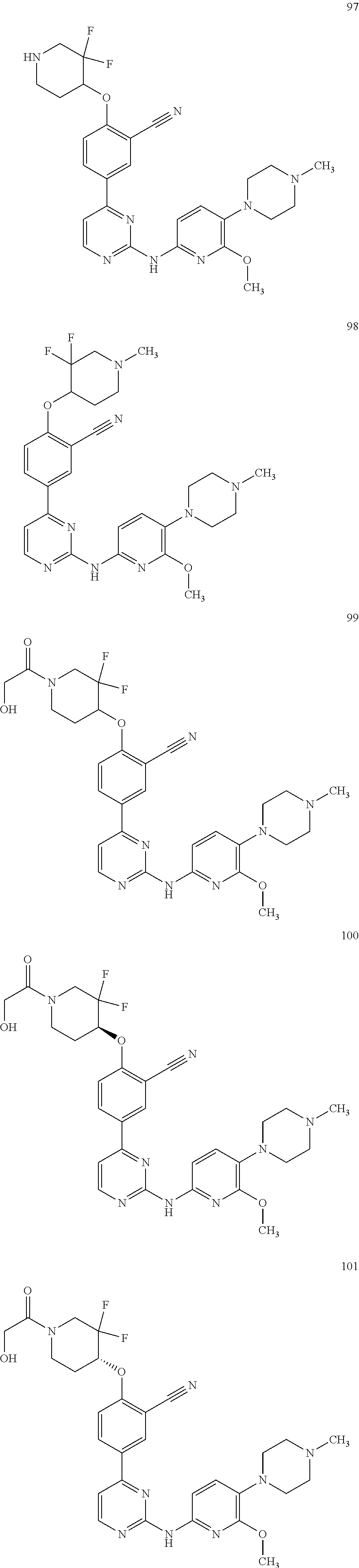 [SMILES: COc1nc(Nc2nccc(-c3ccc(OC4CCNCC4(F)F)c(C#N)c3)n2)ccc1N1CCN(C)CC1 ; COc1nc(Nc2nccc(-c3ccc(OC4CCN(C)CC4(F)F)c(C#N)c3)n2)ccc1N1CCN(C)CC1 ; COc1nc(Nc2nccc(-c3ccc(OC4CCN(C(=O)CO)CC4(F)F)c(C#N)c3)n2)ccc1N1CCN(C)CC1 ; COc1nc(Nc2nccc(-c3ccc(O[C@H]4CCN(C(=O)CO)CC4(F)F)c(C#N)c3)n2)ccc1N1CCN(C)CC1 ; COc1nc(Nc2nccc(-c3ccc(O[C@@H]4CCN(C(=O)CO)CC4(F)F)c(C#N)c3)n2)ccc1N1CCN(C)CC1] 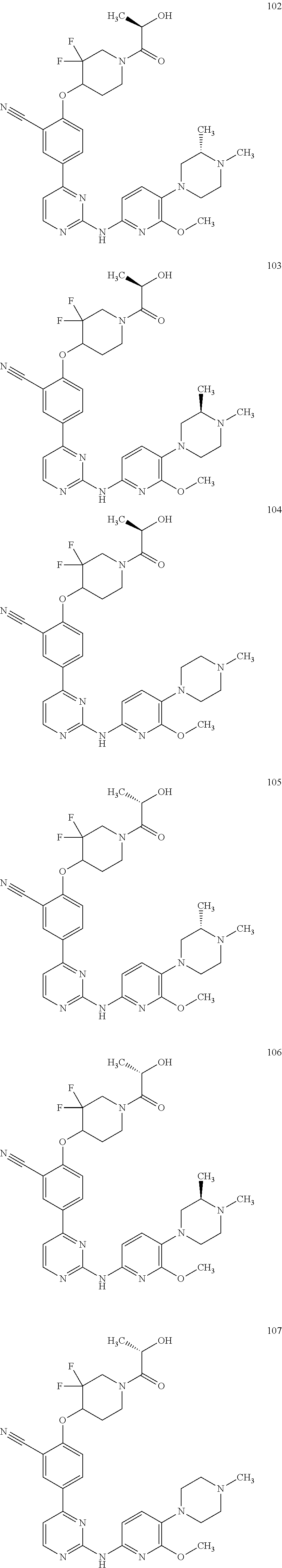 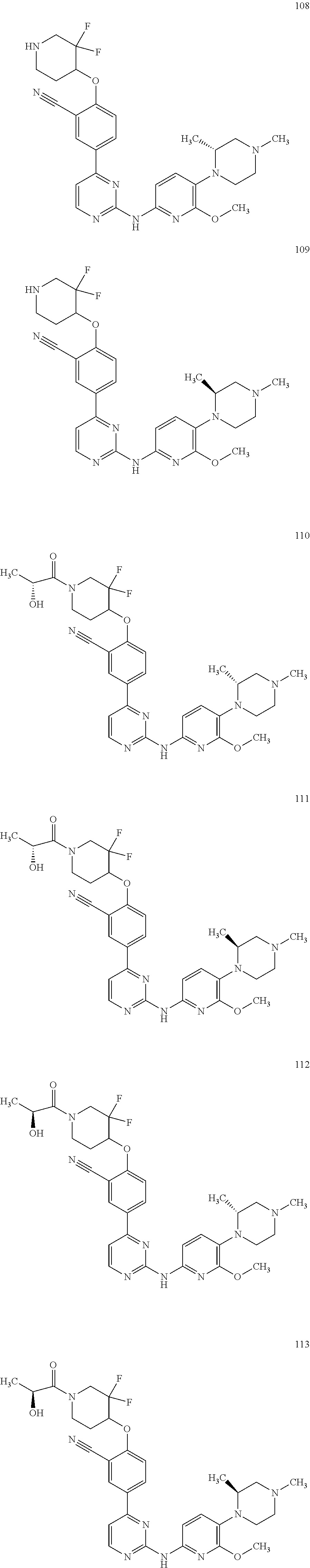 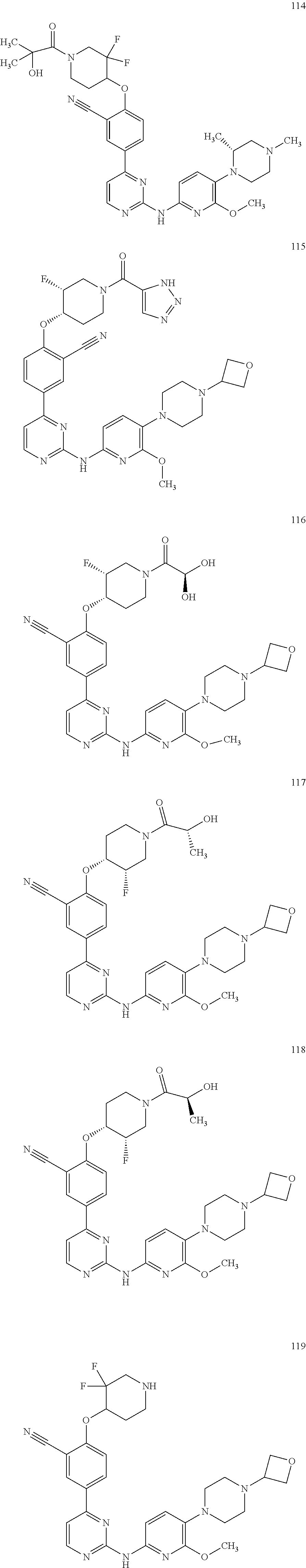 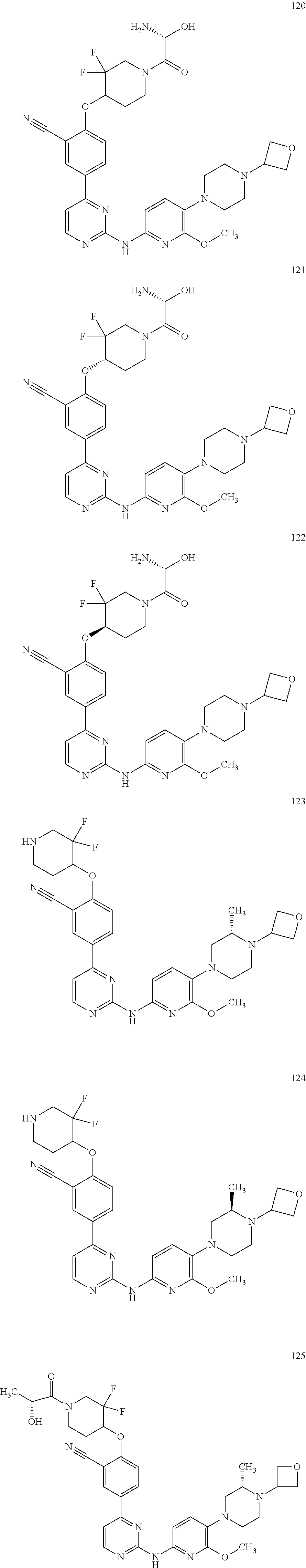 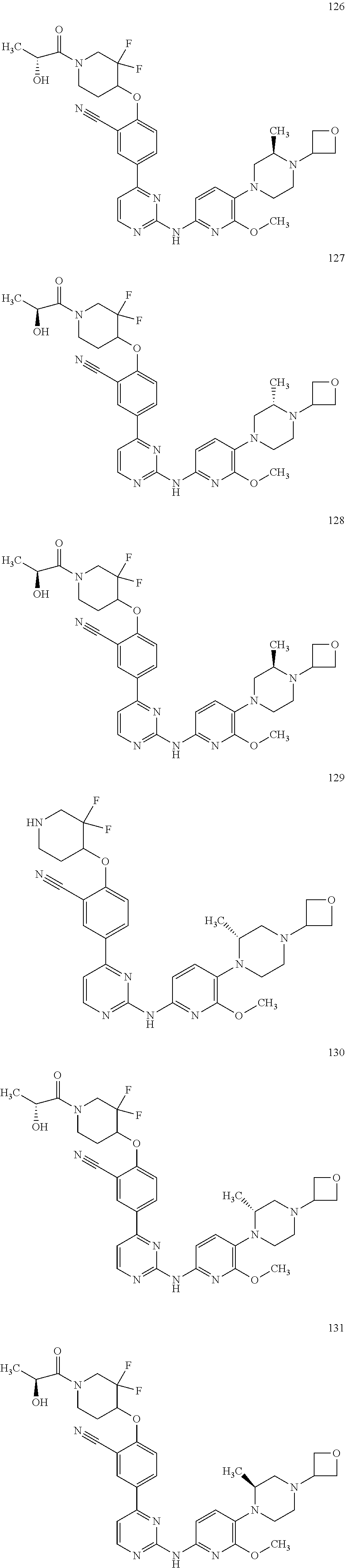 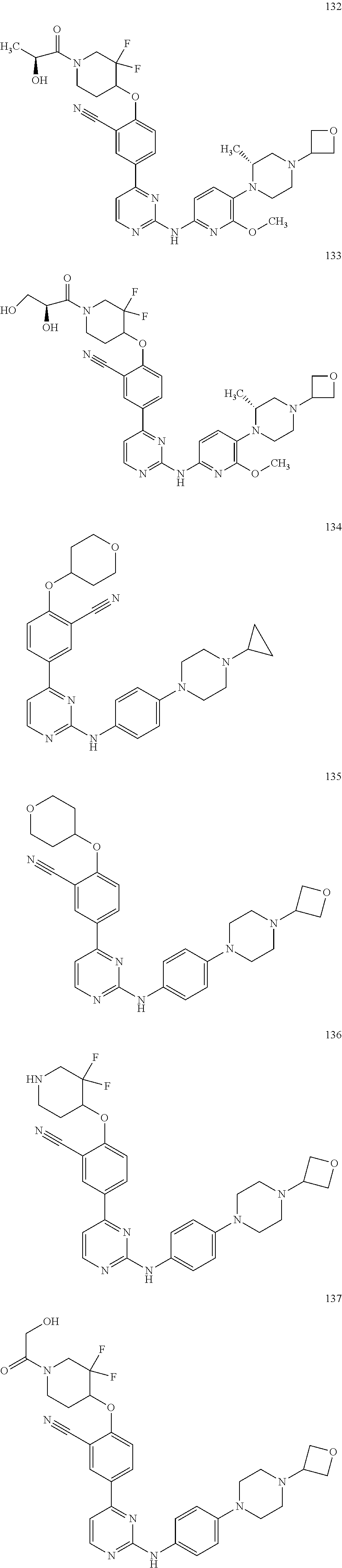 [SMILES: COc1nc(Nc2nccc(-c3ccc(OC4CCN(C(=O)[C@H](C)O)CC4(F)F)c(C#N)c3)n2)ccc1N1CCN(C2COC2)C[C@H]1C ; COc1nc(Nc2nccc(-c3ccc(OC4CCN(C(=O)[C@@H](O)CO)CC4(F)F)c(C#N)c3)n2)ccc1N1CCN(C2COC2)C[C@H]1C ; N#Cc1cc(-c2ccnc(Nc3ccc(N4CCN(C5CC5)CC4)cc3)n2)ccc1OC1CCOCC1 ; N#Cc1cc(-c2ccnc(Nc3ccc(N4CCN(C5COC5)CC4)cc3)n2)ccc1OC1CCOCC1 ; N#Cc1cc(-c2ccnc(Nc3ccc(N4CCN(C5COC5)CC4)cc3)n2)ccc1OC1CCNCC1(F)F ; N#Cc1cc(-c2ccnc(Nc3ccc(N4CCN(C5COC5)CC4)cc3)n2)ccc1OC1CCN(C(=O)CO)CC1(F)F] 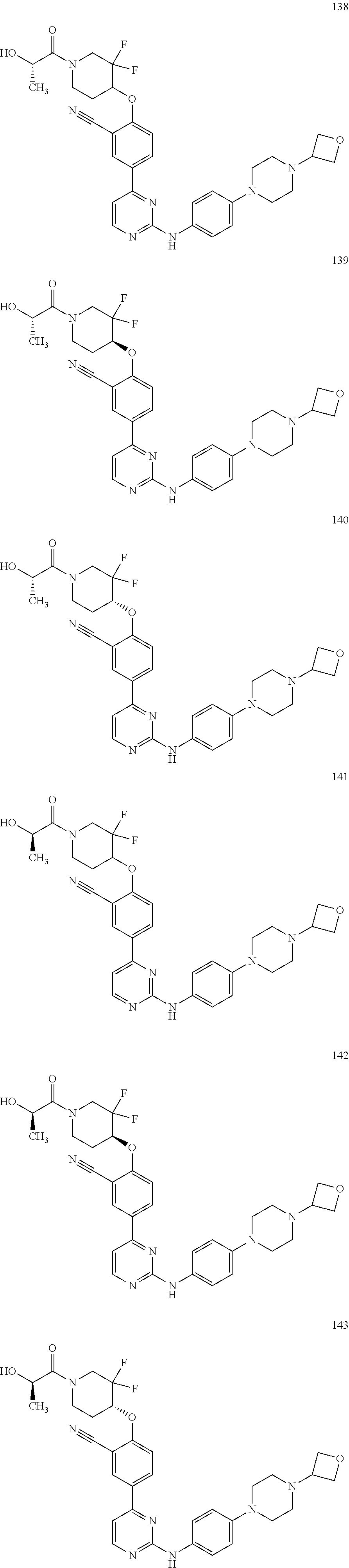 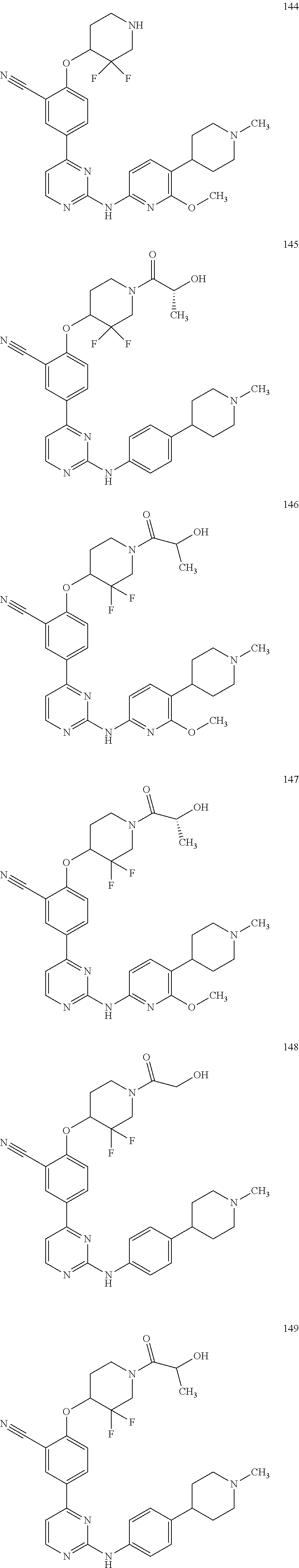 [SMILES: COc1nc(Nc2nccc(-c3ccc(OC4CCNCC4(F)F)c(C#N)c3)n2)ccc1C1CCN(C)CC1 ; C[C@@H](O)C(=O)N1CCC(Oc2ccc(-c3ccnc(Nc4ccc(C5CCN(C)CC5)cc4)n3)cc2C#N)C(F)(F)C1 ; COc1nc(Nc2nccc(-c3ccc(OC4CCN(C(=O)C(C)O)CC4(F)F)c(C#N)c3)n2)ccc1C1CCN(C)CC1 ; COc1nc(Nc2nccc(-c3ccc(OC4CCN(C(=O)[C@@H](C)O)CC4(F)F)c(C#N)c3)n2)ccc1C1CCN(C)CC1 ; CN1CCC(c2ccc(Nc3nccc(-c4ccc(OC5CCN(C(=O)CO)CC5(F)F)c(C#N)c4)n3)cc2)CC1 ; CC(O)C(=O)N1CCC(Oc2ccc(-c3ccnc(Nc4ccc(C5CCN(C)CC5)cc4)n3)cc2C#N)C(F)(F)C1] 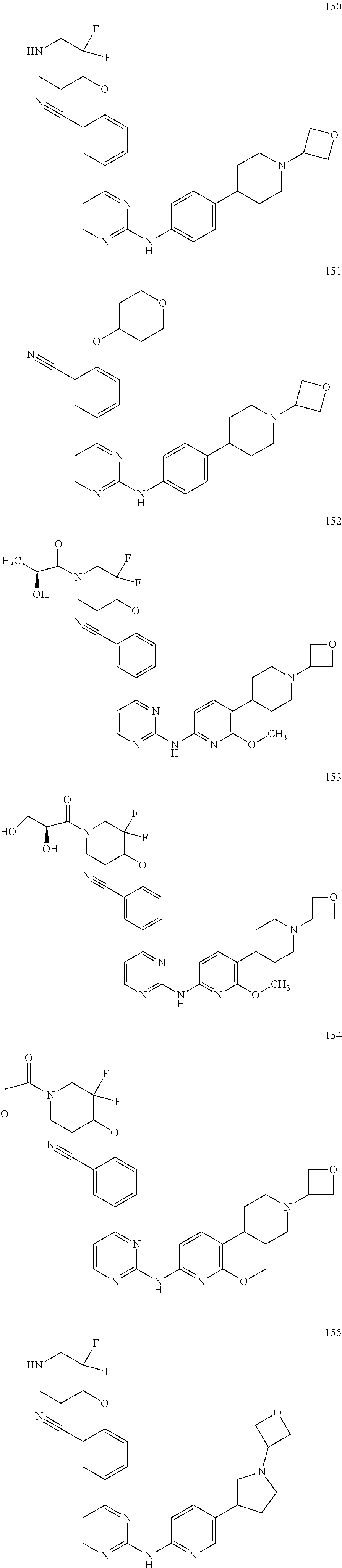 [SMILES: N#Cc1cc(-c2ccnc(Nc3ccc(C4CCN(C5COC5)CC4)cc3)n2)ccc1OC1CCNCC1(F)F ; N#Cc1cc(-c2ccnc(Nc3ccc(C4CCN(C5COC5)CC4)cc3)n2)ccc1OC1CCOCC1 ; COc1nc(Nc2nccc(-c3ccc(OC4CCN(C(=O)[C@H](C)O)CC4(F)F)c(C#N)c3)n2)ccc1C1CCN(C2COC2)CC1 ; COc1nc(Nc2nccc(-c3ccc(OC4CCN(C(=O)[C@@H](O)CO)CC4(F)F)c(C#N)c3)n2)ccc1C1CCN(C2COC2)CC1 ; COc1nc(Nc2nccc(-c3ccc(OC4CCN(C(=O)CO)CC4(F)F)c(C#N)c3)n2)ccc1C1CCN(C2COC2)CC1 ; N#Cc1cc(-c2ccnc(Nc3ccc(C4CCN(C5COC5)C4)cn3)n2)ccc1OC1CCNCC1(F)F] 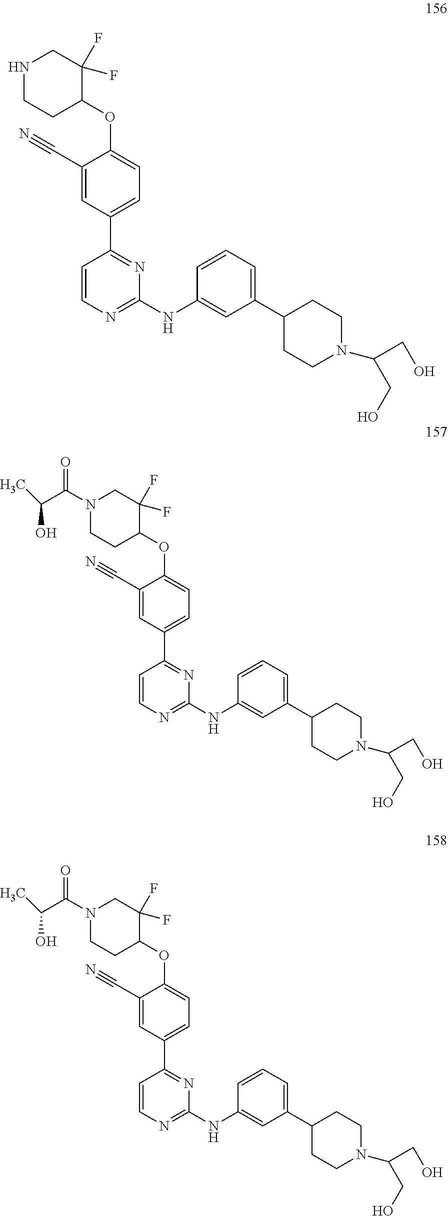 [SMILES: N#Cc1cc(-c2ccnc(Nc3cccc(C4CCN(C(CO)CO)CC4)c3)n2)ccc1OC1CCNCC1(F)F ; C[C@H](O)C(=O)N1CCC(Oc2ccc(-c3ccnc(Nc4cccc(C5CCN(C(CO)CO)CC5)c4)n3)cc2C#N)C(F)(F)C1 ; C[C@@H](O)C(=O)N1CCC(Oc2ccc(-c3ccnc(Nc4cccc(C5CCN(C(CO)CO)CC5)c4)n3)cc2C#N)C(F)(F)C1]
or a pharmaceutically acceptable salt thereof.

16. A pharmaceutical composition comprising a compound of claim 1, and a pharmaceutically acceptable adjuvant, carrier, or vehicle.

17. A method for inhibiting TBK and IKKε activity in a patient, comprising a step of administering to said patient a compound of claim 1 or a pharmaceutically acceptable salt thereof.

18. A method for treating a TBK/IKKε related disorder in a patient in need thereof, comprising the step of administering to said patient a compound of claim 1 or a pharmaceutically acceptable salt thereof.

19. The method of claim 18, wherein the disorder is selected from Rheumatoid Arthritis, Psoriatic arthritis, Osteoarthritis, Systemic Lupus Erythematosus, Lupus nephritis, Ankylosing Spondylitis, Osteoporosis, Systemic sclerosis, Multiple Sclerosis, Psoriasis, Type I diabetes, Type II diabetes, Inflammatory Bowel Disease (Crohn's Disease and Ulcerative Colitis), Hyperimmunoglobulinemia D and periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, DIRA (Deficiency of IL-1 Receptor Antagonist), Alzheimer's disease, Parkinson's disease, and Cancer.

20. A method for treating Systemic Lupus Erythematosus in a subject, comprising a step of administering to said subject a compound of claim 1 or a pharmaceutically acceptable salt thereof.
